SEQUENCE LISTING (264 sequences)

SEQ ID NO: 1 - DNA, 567 bp, Homo sapiens
ttaagttagatgattttctgaggtctcttctaatggttaagatttttatcattttctatttcataaggctttcagctagcagccttaataaaaaccagtgcctggaacatgacctggcctgtagtgacactcagtaaacgttgagtgaataaatgattgaaacacaccagaaacaagtgcatttgagtgcttttacacacygtgcttagtgcttaatgtagattacctcatttaattatcacacagtgccaaggtagatatttctacccccattaaataaacgaggagacggaatagcttctttaaagtcacttacctagtaagtgataaagctgaaattcaaacccagataaatttcactccaaagacttctgtttctgttatattgctatttgtaaaatcaatttgtgtcctagcaacgtcgtctttccaggatacctttagaaaaattaaagctttcttcttgtcatattcttttgaaaagcttgcagaccatatatttaaggtttcaagtgactggcccacatctagttgttctcctaaaaatgaaattgtcaacttaagaga

SEQ ID NO: 2 - DNA, 458 bp, Homo sapiens
ttttttctattctcaaaagaaaggtagcagagagggtgacttcaggcttcttttatgctgtaatactttagtatagtgtattattttgatgcttgatggttggttaaatctttaatatattttctttctttttttaaatatattttcatgtgttctaattcaagggttgttgggttagttcattagttcagttgtatatargagttatgtttggtcaatgtatttgtctccttttctcacatacatgagttttgaacaattagtatttattgtgcacaagaaatgctgatggggccatttttccagtttacatattgaggaattatattttttaaagtttccctcttccctttcttcctccctctctctctctctttcttatccacattttactcagacctaagatctttaactataggagattttcgtattaaatctaatgcaaaacattcatgttt

SEQ ID NO: 3 - DNA, 400 bp, Homo sapiens
tgtggggaagaatctctttccctaagttgcacccttctgacaactcaaactgtagctgtcagggctggattttttttttttttcatctcctgccggaatggggttctctgttaattttggagagggggtttctgagaaaatggcaaagggtactgtttgtatgacatggagagaaagaaagaaaattatatgggtacatarcacccccattcttccctaacaccttgtctctattttgccctagatgaggtgcttaactagcattgggtatggtttgggtgatgtcatgacagtggcaggatatgaataggatgtattctggtcagtttattttctacatcaaacaccttatatgaatctagcctttgtgaagacttcatgacaagctggcatatgagca

SEQ ID NO: 4 - DNA, 400 bp, Homo sapiens
tggccagcctactattctttatagcctggctttgcttcacttttctactggtgcttgtgatagaacaatgaaccaattaatttttttaaaattccatccttaacatgtaaatgaggaggaaaccaggtcatttgccaaataaggaaaattcaagcttccaagggagtttcaaaaaacaatggaggatcaagttcaattgtrggagactttttgaaattctttttcttctaatacatattgcttaatgaaggtactcctgggcattccacatatttcaaaaatgtagtcactgaaccagaacttgaatcagttgtctgaatttctctggattgtggggctcagagtcttctccagccaatgatctggggtgaaggaagttaaagaaggcttcttcaactga

SEQ ID NO: 5 - DNA, 550 bp, Homo sapiens
gctattagatctaataagtacatttagcaagatcacagggtacaaagtcaacaataattcataattctatatactaacaataactacttggaaattaaattttaaaacacagtactatttaaaatagtgtgaaaaatatgaaatcttttgggtagaaatcttacataatatatacaaattctatatgctgaactaataaamtgctgatggaagaaatagaagacctgaataactggagagatatatttgtttattgatttggaagactcatcagagtaaagatgtcagttctccccaaattgattgaaagatccaacacaattctaattaaactcccaataggatttttttgtagaaatcattaagttgattctaaaatttatacagaatgacaaaggaactagaatagccaaaacaattttgaaaaagaacaaagttggaaaagccacacaacctgataaagttatcatattcaaaatagtataatattatagaaaggatagatctaggccaggtgcggtggctcacgcctgtaatcccagcactttgg

SEQ ID NO: 6 - DNA, 500 bp, Homo sapiens
caggcctggctgaaattcagggatggcatctagggcttccccactcctgtctggtaccctccacatgtctgagtgtccctccttgtggcaaggggacagccacaaaatgggttccctcttctgagcctctccgctctcagggaggagaaacctgcccagagtccccacccctaaatccctccagactggacaagcaccacyagccggctgccttctttgggttaggccagccaaagctcacccctaagattaggtgtgctcacaggcccctgacaaaatggggtcctgttggtgaggaagaggagggacgggactggctgctgggtcagtaactggggtatttgtccccggccccaggctggaaggcattggtagacttgtacagatcacttcacttgtggggaccctgtggcacagagagaccccgtggcttgtccgggaccacacagctaagccgggcagaactactgagcgaggagcctatcagtcctggttcccaa

SEQ ID NO: 7 - DNA, 550 bp, Homo sapiens
ccagaggaaggccagctgcaaaccactgaccccaatgtgcggcatctgagggacggacacgcccaggggtcaagagaacggagcctgggagtggcatccacagggtctgctgtaggcgactccagtgccttcctcttgatccctctgctcaggtgcctactccagggaggggctggctttttggattaggttggatgatgmccatcctcaagtgtctgaataaagctccttcagagtgaatgcaatggagaaagggtagtgccttgagaggatctcaggatgatagtaggaagggaaataaatgctgtaaagctaagcagccctcacccccacaaatccactgagatcttttcttttcttttagagagggtctcactctattgcccaggctggggggcagtggcacaaacacagctcgctgcagccttcacctcctgggctcaagcgatcctcccatctcagcctcctgagtagctgggactacagacgtgtgccaccatgcccagctaattacgttacccaggctggtcttgaactcctgggctcgaac

SEQ ID NO: 8 - DNA, 400 bp, Homo sapiens
atggctttttaaaatttaaaatcatttaaatggttgagtttaagacttttgctcctaatgaattcatattcatttgggtgttctgcatcttcatggtcagcagttttgctatcctgtctaaatttgatcatcaagactcattcttccagcatgctggcaacattgagactacctctgtgtattcattaatttgtttttcaygagtgaaaaggtttgcattgtttgaagggtgctgaacaaagttgtgatactataattttttgtttatctgctgtgaatactatttattagaatttttaaatacttatttgcctctatttttctttaggtttgacaggggttagtttttaaaaaatatattcttttttaggcatatttaatttaatttcagtaatcaatt

SEQ ID NO: 9 - DNA, 400 bp, Homo sapiens
aatcaattcattgtgtatgagactgtgtttctagttgcattttcatattgctaccaaaaactagacattattttgtatggaatattaatggaaacatgctgtactaaaatatgcaggtctgattcccagaaatacaacagaagttatatttttaaaggaaaaatcataaccaccctagctttatattttgttgttagtttyttttattttcatttctaacataagtaagacttgattggtttaaaagtcacataaaatgcggcactatttctgaacaaagagagctcatcatcagtcttaatattcagagaaaacttcagagaaattatgttttcatccattaaaattaatttgtgcatcagaaaatgcagccttaaacagtgtccaggagatgggatgg

SEQ ID NO: 10 - DNA, 423 bp, Homo sapiens
catatcaagaagaatgtgagtattttggaggtccatcctagttataaggaaacttcaaaccgtatcatgagagaaatgttgaaaataactgtttctactgaagaaacagtaaaggctctagatttcaaatattttgagagtcattatgtgtaacaggaattagacttgttctgaatgttcctaaagaatggaatgagtgtyaaagtttgtaaatttacatttatttgcacgattacttgttttatatgtttcccctccgctggtgtctaagcttcctgatggcaaaagttagatttggtcaccaatttatccccagtgcctaacatgcataagagccacttatataatggttaacagactgagagaaattttttttattctctagtgtaggagttagggtacaaaataagttgttataacaaa

SEQ ID NO: 11 - DNA, 401 bp, Homo sapiens
tcttcagaccctccaataaaacttatttaatcctaaatgggtcctgttaaaaattccttcattattttgtcatgctttaagacccaggcaaaactcttggtgggcttttgttaaattccagcctttgtataagggcactggcttttaatatttaacttaaccactcagccagtactgaaacagttgttatggaggcctgcrttagtgagatctggcttgccacacttgtgttacccactctttccagagtatactttcttcccttcttcaccttttcaaatactcatctttttaggccctcttcaggttttctgcatgtttccttataatatcttcaacctctagtcagaatttgtttccttccctttgttcccattgctttattttcattgttaggacat

SEQ ID NO: 12 - DNA, 401 bp, Homo sapiens
caggcaaaactcttggtgggcttttgttaaattccagcctttgtataagggcactggcttttaatatttaacttaaccactcagccagtactgaaacagttgttatggaggcctgcgttagtgagatctggcttgccacacttgtgttacccactctttccagagtatactttcttcccttcttcaccttttcaaatactyatctttttaggccctcttcaggttttctgcatgtttccttataatatcttcaacctctagtcagaatttgtttccttccctttgttcccattgctttattttcattgttaggacatgacttacagcctgatgtaagtttctgttcattgtataaacctctgcctttcccagtttattgcagatcctttagtaactaggat

SEQ ID NO: 13 - DNA, 401 bp, Homo sapiens
tgaggattggttccaggatcccctcccccctaccaaaatccaccaatattcaatccctgtatatttgcatataaccagtttacacgaatcatcccatttactttaaattatctttagattacttacaaaacataatacaatgtaaatactatgtaaattatactgtattatattattatttttgattttttcaattttttwaaatctgccattcagtctatagatctggaacctgtagatacagactaactgtatttggataatttcataattttaatgagagaaaggggagaggggaaagcctggtttactgcccatgatgaagtagtaatacagtaaatttagttgagacatcagccaaccttttttgaatacctactaagtacctggctgagagagtt

SEQ ID NO: 14 - DNA, 401 bp, Homo sapiens
tccatttttctctttttgaattttttccttttcacattactttagtaatttgttcttcatctcttatttttatcacctagacagaaaatatagcaaagcataaatcatttttcaggtcaccatgcttcattcttcttttattggggaaggggcagtggtgatccgggaagaagcatagtgtaaacattttaatacaaattyctctttttttttttttttgagatggagtcttgctctgtctcccaggctggagtgcagtggcacgatctcggctcactgcaacctctacctcccgggttccagtgattctcctgcctcagcctcccgagtagctgggattacaggcatgcaccaccatgcccggctaatttttatatttttagtggagacagggttacacc

SEQ ID NO: 15 - DNA, 401 bp, Homo sapiens
atattcaaatcctggctctttattcactagctctctgattcttaaggatattaccagaatatcttaatatctttagttaaaaacctaaaatgtacattcaaaacttaaaacttttttgaaattagcagtggtctaagataagtggtggtttgagcatattccagccttagtgaggttttgaaaagctgggaactaatggtrtttcttggatcctaattctttactaagggcttgaggccattataggaggattctttccatttcatatttattaacaattttgaatttgcaacactttcatggaagtgttgcctaaagcatgggtccccaatttgcatgtcaaggaccatgctaggaactgggcttcacagcaggaggtgagcagtgggcaagtgagcgtt

SEQ ID NO: 16 - DNA, 401 bp, Homo sapiens
atttgggtccactaattagagttcttcatctttctttttacatgtggatatgtgttgcctttcattcatctgtaatttccaggtccttaaaaaaaaaaagtagattgagaatgcaggcattttgaagactgggtgcaaaaatcctagaattctgcctcccaaccccaacccccaaccccaaggtattaggtttttcttgcscatacctaatttggcagcagtgttattttgaggactcatttttgtaggatctttctgatacataactcagttttcataaaaacaatttttatatttttcatttaatgacacaatatttaattattataaaaccataattacaagtttaactaacataaatcagcttgagaacaaacaactaattcttagagtagagtgcc

SEQ ID NO: 17 - DNA, 401 bp, Homo sapiens
tgctctctgaaatgccctgctaaatgcttctcttaattatttgaataaggtagtttggaataaagaaagaaaagatcactctacatacagatagtaaacttaatttgtgatcctatatatgagacagtataaaaatacagataagttttagaaagactcaaaacaatatgtaaatgactgatgtttgcattattaaggaaracttgggatgttgggtcaagaggggaaagtgttagtcaatccactttggagcaatatcatgaaggtcaattataattccatatacctttctttgatgccacagtcagagatagaatacagtttgggtggccatggatgtgccccaatacagtacacattttttggttaaatttgttttcagatcatttcatggaatcttt

SEQ ID NO: 18 - DNA, 401 bp, Homo sapiens
ggaaagagatggggagaaaaagaaggaacacagtgactgctctgttcaaaataggggtccacatgtccaagatgctgtggctccctgtggcggacatcaacgctctcatccattatgctcctcttctgtgggagggaaacacacctcccatcgtgctgctcttctatgcccagcagcattgattagagaatggattttccwttaaaaaatacatacacacacacacacacacacacacacacacacgcattgcatattagaattagagggatttctggaggaatcaccataccttatttgtacaaggtcagcaatcttttataaaagttgtcaaaagtttatgtagagagagaactgaaaactatgcttccatccgttatctgtgttgggcactgaggttg

SEQ ID NO: 19 - DNA, 401 bp, Homo sapiens
catattagaattagagggatttctggaggaatcaccataccttatttgtacaaggtcagcaatcttttataaaagttgtcaaaagtttatgtagagagagaactgaaaactatgcttccatccgttatctgtgttgggcactgaggttggatggtaagactgtggaacagatttttaaaaaattgcaggaaacagatcatytggttgtggtagtaggtctttacatgagatgatactcatagtctatcttgcttttaattttctatcttaaaaaataaaaaacgttatttttagaaggttgtagagaagcgatccccaacctttttgacactagggaccagatttgtggaaacaatttttccacgaagattgggtggatggtttttggatgaaactgttcc

SEQ ID NO: 20 - DNA, 802 bp, Homo sapiens
ttgtatgctgtgcttcattcatggggctgtgaactactgattatattctccctattcctaatgtagaatgctttattctactgccatctttctgtctgcactgtttaattaggcttactgataacaactttaattctgaattttctttctcattcaggttctatttgtaattactaagacttaaagaatagtctggtgaarttactcgaagaattaaggaaggtttgagctaaaatgaactagagaccatctagtactttagtgtaaaatatgtttaatacaagtcgttaagtccttgtaagtgactattccaatgttcattctttgtttttggaagaatgcttggagttaccatgtttttaaatgtgaaatttcatctaaattaaaaaaaaaatctctgtttgtatgctgtgcttcattcatggggctgtgaactactgattatattctccctattcctaatgtagaatgctttattctactgccatctttctgtctgcactgtttaattaggcttactgataacaactttaattctgaattttctttctcattcaggttctatttgtaattactaagacttaaagaatagtctggtgaarttactcgaagaattaaggaaggtttgagctaaaatgaactagagaccatctagtactttagtgtaaaatatgtttaatacaagtcgttaagtccttgtaagtgactattccaatgttcattctttgtttttggaagaatgcttggagttaccatgtttttaaatgtgaaatttcatctaaattaaaaaaaaaatctctgt

SEQ ID NO: 21 - DNA, 401 bp, Homo sapiens
tcccaaagtgctgggatttcaggcgtgagccacctggcctggactgtaattgaggatttttctgtgtcatattctcaactgttgttggtgtgctacagaaagaggaggaaattttttttaatctctgaggcgagtaaaggaaaccagaatactacaggacacctaattttttcaatcttcatgaaaatgcaagctgtgaakttgaggtttggtatcgtgaagccagagtctgtacagataattcgcagcaattaatgaccacccttcttaataatcttccatcagaaacctttttaagacctcagtggccagttgcagcctacctttgtggcttcatctccagccacactggacagccacccccagtttctgcacatgcactgctctcttgtgttcccgga

SEQ ID NO: 22 - DNA, 404 bp, Homo sapiens
  misc_feature (204)..(204): n= absent or T
tgagagttcaaccaagtaacattgccccactaaacacaatgtttaaacacagtggtatccaaaatgggatgaggaagtgtgcaagaagtgcaatacattagagtgtctattatttcttatctaattttaaattttatattgttataaatttataaacataaatgatatatagtataaaaagttaaataaatacattatttattntttcatgcttttaatttttttaccataccttaacatatgcatataattttttttaattaatttattttttttgagacggagtttcattctcgttgcccaggctggagtgcaatggcgccatgttggctcactgcaacctccacctcccgggttcaagtgactctcctgcctcagcctcctgagtagctgggattacaggc

SEQ ID NO: 23 - DNA, 402 bp, Homo sapiens
  misc_feature (202)..(202): n= absent or T
gtgtgagccaccgcgcccagcccatataatttataaataaaaatatgtatattgggaagttcttgctcaaaaaatctttactgactggagtatgtagtaacaaaaaaagtagggaacactgctttaaacagaaacataaaattaaacataaacattgctgaataactaaccatatttcccaaagaagctgtatctacatttntcattttatagtaaaatttgataagtttcacagctttagaattgtactggatgaatgttattatggtaattcaccgtatctattgtaatacacaagcttatcacatagttattaatatacattaaaaatataatacatgatataataaacataaggtcagtatttctatgactttctatggtgtttctttttattttcag

SEQ ID NO: 24 - DNA, 401 bp, Homo sapiens
ggactaaatttagcctctctgttaaccatctcatattttctgcagcgttaccttcttcagtattttaagccagtgattgacaggcaaagctggagtgacaagggctcagtctgggacaggatgctccgctcggctctcttgaagctggcctgtgacctgaaccatgctccttgcatccagaaagctgctgaactcttctcycagtggatggaatccagtggaaaattaaagtagatgtagacttctgtcctaccctttgttcttttctctttgatgtaaaagtctttgatcaagcaagacattaggtctaaaaccttttagtgaggatagaaaaaaaaacatgctgggcattacaaaccctgtttcatgctctcacattgtaagtgctatgtatggagact

SEQ ID NO: 25 - DNA, 401 bp, Homo sapiens
  misc_feature (201)..(201): n= absent or A
cttcagaagattgctgcccacttgtaaagtaatctgaagactgtcagaaaaggaatagtgcttaaactgtttctagaagctacagacttataattttctgttctgtaactataaccaggctcttctgaatcttagaatcttattgttgaagctttggtccgtctagagattttaatcttagagacatacactagatgtgcngtattaggcatatagactaaataaataaaacataaaagcacataaaagaatagtaatatttaaaatgcataatacagatcaaatataggtaaagggtaaatatgtcaattatatttatgcatcctttttatttgatttatatattttttaaatcttagacctttattgtttctagtggcccactgaagtaagtcaggggc

SEQ ID NO: 26 - DNA, 401 bp, Homo sapiens
tgtagaatttagtagcaaaaacatttgccatcaaagtagactagataatttatggtaatgcttcaagctattttctcttgccaaagcaaatcgtaatcttatccaacatgtcaaacatgcttaataagctgcagtcagcatcatcacaagcctgactcccagaaagggctcagggatagaggtggggaagagcctgtctargagttgtgactagcttgaagaaaatgttttcagattattggatctgtatccattcagtatttgggggcattgtaccatggtgaagaccatctctgagacaagctgcccagaccaaatgaagatagaattcagtcattacccagtgatcttgatagatgcagctgacgagactgcaggctgaaaagtttctgcttcctcag

SEQ ID NO: 27 - DNA, 401 bp, Homo sapiens
atcatcacaagcctgactcccagaaagggctcagggatagaggtggggaagagcctgtctaggagttgtgactagcttgaagaaaatgttttcagattattggatctgtatccattcagtatttgggggcattgtaccatggtgaagaccatctctgagacaagctgcccagaccaaatgaagatagaattcagtcattasccagtgatcttgatagatgcagctgacgagactgcaggctgaaaagtttctgcttcctcaggagatggacagaagcttaaattactaatgacctccttggcctgactgctttcattgctgaatcaatgaagcaaagataaaataagaccatgactcaagctgtcacgcagcaagtgagagaatgagcatcatctttggag

SEQ ID NO: 28 - DNA, 401 bp, Homo sapiens
caatgaagcaaagataaaataagaccatgactcaagctgtcacgcagcaagtgagagaatgagcatcatctttggagtcacacggtcacatccacatcttggtcctaccgtggaactagccatgtgatctccaacaattctgtgaacatttcagagtctctgtttcctcacctgagaaacaacaccaacctcacacccacrtaacaggattaaaagataatgtgcagcctctagttcagtttcacttcctgttttctttttccacaggggtgtacttcttgtacaacaaataaagggaaaggggccattatctggtattttacttaaaagcacagaagttgaattgatgccagtgttggaaattattgcattttaagaaaatagaaatatgtaatattttt

SEQ ID NO: 29 - DNA, 401 bp, Homo sapiens
ttcttgctgtgtcctcacatggtctttgttctgtgcatatgtggagagagcgagctttgctgtttctttctatcaaggacaccaatcctattggattagggctctacccttatgaccgaatttaaccttaattaccatcttaaaagccctgtctccaaatgcaatcacaatgggggttagtgctttttttctttttttggsggggggcgcgggggacagagtcttgctctgccacccaggctggagtgcagtggcgcgatctcagctcactgcaagctccgcctcccgggttcacgccattctcctgcctcagcctcccaagtagctgggaccacaggcgcccacaccacgcctggctaattttttgtattttttagtagagacggggtttcactgtgtta

SEQ ID NO: 30 - DNA, 401 bp, Homo sapiens
  misc_feature (201)..(201): n= absent or C
atattttatttttaaagtaaatttatacaacttttgtaagttctaaattaatttgaatatagtttgttttaactatagtatcagtatatctttaagatattgtaatcaggttatagataattaatatgacacttcagccaattatttaaaaaattcctgaggctgtaaatatcctgtgggttaattgttttctctcccccnagtggtttggcaatctggtaacaatgaagtggtggaatgacctatggctaaatgaaggttttgccactttcatggagtatttctctttggaaaaaatattcaaagagctttctagtgtaagtacagggtttctttggcctactatgaatgctaggaggaaaaatagtcaaatcacattttcatgtattttctgtgcatct

SEQ ID NO: 31 - DNA, 401 bp, Homo sapiens
  misc_feature (201)..(201): n= absent or C
cccttgcctcctcactccctcggcctactcctgtttattcttcagatcttagctcagccattgcttgctccaggaaacctttccttccctgaagacagtttagatgccctacttaggttttttaataacattctctacttctccactcataatatactgtaagtactgttcactcatatctatcctcatattaggtatttnccccattgacggtagtgatcatggctatatgaatcactgtaaatcacttttagcacttagtaggcacacaaaaacttaatgaattagtaaattttagtccataatgaagtgactccagtctcactataaaaatttctaggaaaagaacatgcaaagcctgataaaatgatgttttctttttctcttcctcttcttagtaa

SEQ ID NO: 32 - DNA, 401 bp, Homo sapiens
cttttcttacagtattttatcagtaccttcctctttattggagcttagaaatagatttcaaatagaattcagcaaaattaaattctgtagaatttagtagcaaaaacatttgccatcaaagtagactagataatttatggtaatgcttcaagctattttctcttgccaaagcaaatcgtaatcttatccaacatgtcaaamatgcttaataagctgcagtcagcatcatcacaagcctgactcccagaaagggctcagggatagaggtggggaagagcctgtctaggagttgtgactagcttgaagaaaatgttttcagattattggatctgtatccattcagtatttgggggcattgtaccatggtgaagaccatctctgagacaagctgcccagaccaa

SEQ ID NO: 33 - DNA, 401 bp, Homo sapiens
taaaattctaagcctcccaagtgactgaacagaccatgtcttggccaaggggaccccagggtaaccttgaaaactaaattctcattcatgacaggatgccagggtcaaacaagccttattataccccttcctcaatattcaggattagcctttcttccctaagggctaaacggaaaccagcccttttgaaagattccaccmctaatatcaaccaaccacctgatattgcctctagttttttgcctgataagagatcaccacatggagtggttctggcccatctccagagaatgcacagtaagagttttcatgtcctctgcttcaccttttgatgtcagaggactgaaaactccaccctcggatcatgttaacactgccattttttgtatatgggacccatg

SEQ ID NO: 34 - DNA, 401 bp, Homo sapiens
gtgctgggattatagacatgaaccaccacgcctggctatcttttcatttcttgatactatcctttgaagcatactttgttgatacttatcttcaaccttatttccattacaatgaagttgttatgagttgaatagtgtcctccaaaatttatctgttaaatttataatcccccatatttcagaatgtgaccttatttgaartagggttgttgcagatgtattagttaagataaggtcatactggagtagggtgggcttccaattcaatatgactagtgtccttattaaaagaggaagtttggacacaggtatgcacacaggaagaatgtcatgtgaacactggagttacttgccacaagcctagggactattagaacctaggagataggcctagaacagat

SEQ ID NO: 35 - DNA, 401 bp, Homo sapiens
ttgctcttttgcccaggctggagtgcagtggcatgatctctgctcactgcaaactctgcttcccaggttcaagtgattctcatgcctcagcctattgagtagctgggattacagacacagaccaccatacacagctaatttcttgtattttgtatttttagctaagctggtctcaaacttctggcctcaagtgatccgccyacctcagcctctcaaagtgctgggattatagacatgaaccaccacgcctggctatcttttcatttcttgatactatcctttgaagcatactttgttgatacttatcttcaaccttatttccattacaatgaagttgttatgagttgaatagtgtcctccaaaatttatctgttaaatttataatcccccatatttcagaa

SEQ ID NO: 36 - DNA, 401 bp, Homo sapiens
tcccaaagtgctggaattacagtcctttgcctacttttaattggattatttatcttttatcattaaatttaaaaattctttatatatgctagatacaagtcccttgtgaagtcccttggtttgtaagtattttctcctattctgtgaactgtcttttcatttctttctttctttctttctttgagacagagtcttgctctkttgcccaggctggagtgcagtggcatgatctctgctcactgcaaactctgcttcccaggttcaagtgattctcatgcctcagcctattgagtagctgggattacagacacagaccaccatacacagctaatttcttgtattttgtatttttagctaagctggtctcaaacttctggcctcaagtgatccgcccacctcag

SEQ ID NO: 37 - DNA, 401 bp, Homo sapiens
agcctgggcaacctggtgaaaccccgtctctatgaaaaataaaaaaattagccaggcatggtgatgcatgtctgtagtcccagctacttgtggggctgaggcgggaggttcgcttgagcctgggagatcgaggctgcagcgagctgagactgcaccagtgcactccagcctgagcaacagagtaagaccctgtcttgaaamaaacaaacaaacaaacaaaaatggtagatgaatgttcatagctgcattattcacaatagccaaaaagtataaacaacacaaacgtccatcaactgatgaatggataaatagaatgtgaaacatttatatgtataatagaatattattcaacaataaaaagaaagtactgacatgttaaaacatagatgaaccttttaaaa

SEQ ID NO: 38 - DNA, 401 bp, Homo sapiens
ttgaatgaaatctgtacctttttaaatagtagcaaccagatgggggaaaacaaacttgcttgagcaatggtgaatggagatactcacagtataatttgctttttttttttttttttttttgagacggagtctcgctctgtcgcccagggctgcagtggcgtgatctcggctcactgcaacctctgcctcccaggttcaagygattctcctgcctcagcctcccaagtagctgggactacaggcgcgtgccaccacgcccgactaattttttgtatttttagtagagatggggtttcaccgtgttagccaaaatggtctcaacctcctgacctcatgatctgtccacctgggcctcccaaagtgctgggattacaggcttgagccaccatgcccagccatta

SEQ ID NO: 39 - DNA, 401 bp, Homo sapiens
aaccatagcaaacgcccatttgcctccgaaccatctctgccaccagccttctagtagcccagacgtatttccccatagtctcacagcctcacgcctctgccagtaacccctccacacacttgactaaatggttttgctgctgagtttggtcagaagaccacaataataccccagctctcagcccctaccataagacagcayctcctctgctgggagtggatatccagagaacactggttgaatcagcttcctaaaaatggagacggttgttggggaaaattaatttgctggatagagttcttaaaaattacagccctgtatatactttgacttttcttacagtattttatcagtaccttcctctttattggagcttagaaatagatttcaaatagaattca

SEQ ID NO: 40 - DNA, 401 bp, Homo sapiens
tgctaaatctgggtactggaaaggataaagagagggcagagcaaaggccagaggtttcatctttgtggaaggtctgtattcagagcagagaggaagttgaagcccaactcaaacaggcagataaagagagatcaaagagatgagcatgagatacagtcccctcgtgcccaaggagacagggtggttacagacatggaaaakctgagaataatcacctctgataaagatcacagaagctgcccgggaggtgtttggtaagcttggagttacgtttgtggggtggatgggcagaagtcagatttcatagcactgaggatgcagcacaaggagaagttcaagatcaattcctaagacaacaacttggcactaaaaaacataaactatgttctgaaggctttacc

SEQ ID NO: 41 - DNA, 401 bp, Homo sapiens
ttgagagagagcctcgctctgtcgcccaggctggagtgcagcagcacgatctcggctcactgcaacttccacctccctggttcaagcgattctcgtgcctcagcctcccgagtagctaggactacgggcatgtgccaccatgcccggctaatttttgtatttttagtagaggtagggtttcaccatgttggtgaggctggyctcgaattcctgacctcaggtgatctgcccaccttggcctcccaaaatgctggcattacagacctgagtcactgtgcccggtcctgtttctttatctgaacactaaggacttgtactagctggcctttacaacccttactagttctaaagttaaagactgtgtgagtaaagcttttctctctactcttatcaatcaagta

SEQ ID NO: 42 - DNA, 401 bp, Homo sapiens
gggcaacatggtgacacattgtctttcaaaaaaaataaaatatggccaggcgcagtgacccacgcctgtgatctcagcactttgggaggctgaggcaagtggatcacctgaggtcaggagttcgagactagccaggccaacatggtgaaaccccgtctctactaaaaatacaaaaattagctgggtgtggtggcacatacytgtaatcccagctactcgggaggctgagggagaagaatcacttgaaccccagaggcagaggttgcagtgagccaagatagtgccactgcattccaacctggacaacagcgagattccgtctcaaaaacataaataaatgaataaaaataaagtaggctggtcacagtggctcacgcttgtaatcccaacagtttgggagg

SEQ ID NO: 43 - DNA, 401 bp, Homo sapiens
cagcactttgggaggctgaggcaagtggatcacctgaggtcaggagttcgagactagccaggccaacatggtgaaaccccgtctctactaaaaatacaaaaattagctgggtgtggtggcacatacctgtaatcccagctactcgggaggctgagggagaagaatcacttgaaccccagaggcagaggttgcagtgagccragatagtgccactgcattccaacctggacaacagcgagattccgtctcaaaaacataaataaatgaataaaaataaagtaggctggtcacagtggctcacgcttgtaatcccaacagtttgggaggattgcttgagtttaggagtttgagaccagcctgggtaacagggagacccccatctctacaaaaaaggtagccga

SEQ ID NO: 44 - DNA, 401 bp, Homo sapiens
ccgtctctactaaaaatacaaaaattagctgggtgtggtggcacatacctgtaatcccagctactcgggaggctgagggagaagaatcacttgaaccccagaggcagaggttgcagtgagccaagatagtgccactgcattccaacctggacaacagcgagattccgtctcaaaaacataaataaatgaataaaaataaartaggctggtcacagtggctcacgcttgtaatcccaacagtttgggaggattgcttgagtttaggagtttgagaccagcctgggtaacagggagacccccatctctacaaaaaaggtagccgagtgtggcggtgtgtgtctgtagtcccagctactctggaggctgaggtgggaggatcacttgagcccaggaagttgagg

SEQ ID NO: 45 - DNA, 401 bp, Homo sapiens
agtattctatagtttgcccaaccagttttacgtccaaggaaaattagccaatgcataaaatatacaaactatgaaaggcaaggatcaggaaaccagagactttgccaccaaatctcagattattagaaactaggtgtcagggtttatcaagaaggccaggaaggccttttgggttaagccttacattcatgaagaacctcragggtagatttttgagagcattccaaatgaatggtctctggtcaaatgaatgaatggtcaaatgaataaatctgccctcacagagatacaaaaggaaaaggaatataattcataccatttggtttaagccttacattcatgaagtacctcaagggtagatttttgagatcattccaaatgaagtcgaatctgccctcaca

SEQ ID NO: 46 - DNA, 401 bp, Homo sapiens
atcaagaaggccaggaaggccttttgggttaagccttacattcatgaagaacctcaagggtagatttttgagagcattccaaatgaatggtctctggtcaaatgaatgaatggtcaaatgaataaatctgccctcacagagatacaaaaggaaaaggaatataattcataccatttggtttaagccttacattcatgaagwacctcaagggtagatttttgagatcattccaaatgaagtcgaatctgccctcacagagacacaagaaaggaatataattcatacactattgcatttttaataaatcttttgaaatttgcagaattagattgtattgtgtattttcggttaaatgataattgaatgtaaatatttagatgcagcaccatattttataaccc

SEQ ID NO: 47 - DNA, 401 bp, Homo sapiens
cataatgaaatacttcaagtgaaatttgatgggttgatgatcctgggcacatacctaactctctgaagttcagtgtccccatctataaaattaagttaataatagttctgtttcataaagctgttctgaggattatggatagggaaagtgtgcggatcacatagtaagcactcaataagtattagttattaatgatgatgwcaacggccactacaactacaagaaatactactatttcttgcaaaataacttatctaagggccatctatcaacactgtattacatacaaatgtggaattgtaaaactaggtctatagatattggagactattccctctatttcatttctgaaaactctcagtaatgcagtaaattattaaagtcaccaaaattgtctttca

SEQ ID NO: 48 - DNA, 401 bp, Homo sapiens
ttcttttttccctctcatttagttcttttttagtcttgatttccccacggagagtctcatctattcacatattctcattttttcctttttaaaatacatcttcctgcttaatgatggggatacaattgaaaaataataaaacacgtcttcttcagggattcttttttatttataatggctactctaaagactcactaaatrcaatgcaatatctggaccaccttaagattgctttctaatgattttgtttacttagggttcacattttcttgtttcattaaatgtctagtaattttttattacatattgaatagtgtcaatggcacatggtagagatgctgaattaaaaaaaactctgtaaaatgttgatttttctctctctgtctctgtagacagcttag

SEQ ID NO: 49 - DNA, 401 bp, Homo sapiens
caacaattctgtgaacatttcagagtctctgtttcctcacctgagaaacaacaccaacctcacacccacataacaggattaaaagataatgtgcagcctctagttcagtttcacttcctgttttctttttccacaggggtgtacttcttgtacaacaaataaagggaaaggggccattatctggtattttacttaaaagcmcagaagttgaattgatgccagtgttggaaattattgcattttaagaaaatagaaatatgtaatatttttatgctttcaatcaacaaaatgagatttggcatttttgtgctttggggatctcaaaagcagggctttttgttttcaacagagtgttggggtaaaagcaatggaggtaagagaggctacagaatactaggaga

SEQ ID NO: 50 - DNA, 401 bp, Homo sapiens
agccaggagttgaggttgaagtcaccattgcagatgcttaagtcaactattttaataaatgattaccagttgtttaaaaaaaaaaaaaagaaaactatagagagctatctaccttttgggactaccatggtagcagtcatttgctgttcctttttttgggagggacgggaacagggtcttgcttggctggagtgcagtggyacggccacagcactgcagccttgacttctcaggctcaagcgattctcctgcctcagcctcccgagtaactgggaccacaggtgcacaccaccatgcctggctaatttttgtattttttgtagagatggagttttgccatgttggccaggctggtctcgaactcctgggctccaatgttctgcctgtcttgacctccccaa

SEQ ID NO: 51 - DNA, 401 bp, Homo sapiens
ataccttgtagcctacatagtttgtgatttccactctctgagtggcttcacttcatcaggggtcaaggtgagactgagttctaacgttctacgcagtgcagaaaagtgtcctgagagcaatgaacttttgttttctcatgtttttcattgttatcaaagtattatgtttatattacaagaagagatagataaaaaactaarttaaaaattatccatagtcctgtcaccaagatacaactactgataatattaatgtaagccttccaaatattttctatatgtatgtcagcatatatgggtgtacatagtaacagtatttacttactatatatgtaaaggtaattttcaaagtatatatatatatatatatatatatatacacacacacacacacacacaca

SEQ ID NO: 52 - DNA, 401 bp, Homo sapiens
attcagccaactacttttaaaatttatctttttttttttttttttttttttgagaccaagtctcactcttttgcccaggctggagtgcaatggtgtgatcttggctcaccacaacctctgcctcctgggttcaagtgattctcttgcctcagcctcccgagtagctgggattacaggcatgtaccaccacacctggctaayttttgttttttagtagagatggggtttcaccatgttggccaggctggtctcgaactcctgacctcaggtgatccacctgccttggcctcccaaagtgctgagattacaggcgtgagccaccgtgcctggccaaaatttatcttaattcagactttacaattgactttattaaataaatatttttaagtagaagaaatgtt

SEQ ID NO: 53 - DNA, 401 bp, Homo sapiens
aaatcatttaacttctttagccacattgtggtcacttgtaagatgaggatttataatttttgtcttactttacctattgtttgaaaataaagtgaacaattatgcagaaaagtagaaaataaccttttagaggttggcagagaaatgcctatacctgtgtgtatgtaatttgcaagctcttttgaaaatttttggaagacraagtggttttattgtttctttatttttgaaactgcctcgctctgtcagccaggctggagtgcagtggcaccatcttggctcattgtaacctccacctgctgggttcaagcaatcctcccgcctcagccttccaagtagctgggactacaggcatgcaccatcatgtccgactaatttttgttgttgttgttgttattttt

SEQ ID NO: 54 - DNA, 401 bp, Homo sapiens
gggttcaagcaatcctcccgcctcagccttccaagtagctgggactacaggcatgcaccatcatgtccgactaatttttgttgttgttgttgttattttttgtagagtcaggggttctggcatgttgcctaggctcgtattgaactcctgagctcaattgatctgcccaccttggcctcccgaagtgctgggattacaggygtgaaccaccacactcggccaagacaaagtgttagtaatttttttcttcaatattttacaggtgaaactattttttgaatctcttgaggctcaaggatcacatctggatatttttcaaactgttctggaaacgataaccaaaaatataaaatggctggagaagaatcttccgactctgaggacttggctaatggttaata

SEQ ID NO: 55 - DNA, 401 bp, Homo sapiens
aacttttgcaggttcatgcacagatttaagggatcctcttttctgattctctcccctctgggatttcccccatgctgtatagcctacagggtctacttctggtttctctggatagaaatatgggactcattggaattttacctgttggcatttccacaccactctgtgaccaaagcctgccttcagggcaaagtagagaarggaaatggaacaatattaaaacagaaactcacccctgtgtgttttgcttcagcaagtttttgaccctataacctattataaagtgaaatgaaaatgtggacacctgtgaagcggtccgagtgcaaaatttgtctagactttcaatttttttccccagtcttttagagttgtctcctacctaattcaacaacaattttagt

SEQ ID NO: 56 - DNA, 401 bp, Homo sapiens
gcatgaaggagagctgccaagttctgtgtcttgataacctttccttccattcctagttcaattaacctgaagaaagaaaaataatttgtttctaaatagtaggtattatgtacatggacattaactcaagccaccaatatattaaaagaatagaacagaaagaggcatgataaaagtataattaccaattttttaatgttycaattaaacttttacttttttagaaataatttttattttgttcctatcaaaaacatttacttattatttcaaataagtttgattagcatcatttacacatcttatatgcaagaatgtatttttacaacaataatttttcttcaagtttctgaagataaaacataaccgcttgtctagtctcaatcatatgattaataact

SEQ ID NO: 57 - DNA, 401 bp, Homo sapiens
ctaaatagtaggtattatgtacatggacattaactcaagccaccaatatattaaaagaatagaacagaaagaggcatgataaaagtataattaccaattttttaatgtttcaattaaacttttacttttttagaaataatttttattttgttcctatcaaaaacatttacttattatttcaaataagtttgattagcatcrtttacacatcttatatgcaagaatgtatttttacaacaataatttttcttcaagtttctgaagataaaacataaccgcttgtctagtctcaatcatatgattaataactagggaatacctgttttcactatttgcattttgtcaatatattcttttctgaaagtaaagttaaagccatacacattctgattcaattatct

SEQ ID NO: 58 - DNA, 401 bp, Homo sapiens
aattaccaattttttaatgtttcaattaaacttttacttttttagaaataatttttattttgttcctatcaaaaacatttacttattatttcaaataagtttgattagcatcatttacacatcttatatgcaagaatgtatttttacaacaataatttttcttcaagtttctgaagataaaacataaccgcttgtctagtmtcaatcatatgattaataactagggaatacctgttttcactatttgcattttgtcaatatattcttttctgaaagtaaagttaaagccatacacattctgattcaattatcttatgcctttaaaactggtggctgaagttttagtgacttcactgaatttgtgtcagtttacttataacaatttagttaaattattgaac

SEQ ID NO: 59 - DNA, 401 bp, Homo sapiens
aacattgtcagtggtggtagtgacgatgatgaacttggttttactttttcagtgtctaacctggtcccgtgctaggaccccaggcagagcttcctatgaagtcacgtacaacaaggcctttgggcttaagagaaaaagctcacagctgcacagagggaggagtttttatataaagaatacaaaatgttctgaataccaagwgtttcattctctccttatgtttctgagttgaaatttgaagtaatcatgagacactgcatgtgttcccatttcaaagatgccacagaatcataaaactagtatgtagcatataatttcaattttgtcttaggggataaattagtctaagaatagtataccaaaacattaattgtgataatagtgtagtgatcaatttatga

SEQ ID NO: 60 - DNA, 401 bp, Homo sapiens
atcatcacacccagaccaatgggagcattatacatgcattattttttgtactcaaaaggatgaagtatataactgtctactgtaccatgtcatttgaaaacactagaaaattctacagcaatccttcaaaagttttcaaataaataccctgtccatcttaaacctgaaaagcacttcaaattgctaacatttaatttcttrttgactgtagatattgtcgtttctgttttccttgtaaaaggatgctaaataaggctccaaggagtgattgctacattaaccaaaattatgcactgccaagctgtctccagcatcaactctgaaagataggaaagaatcagaaatccaatttcctacatgaaagttgaagcattgcttctttttttgttctcttctgtggg

SEQ ID NO: 61 - DNA, 401 bp, Homo sapiens
atccctccccagtgcagttcacaatagggttcatgctcctatgggactctaataccaccctgatctgacaggagaggcgcccaggcggtaacgctcacttgcccactgctcacctcctgctgtgaagcccagttcctagcatggtccttgacatgcaaattggggacccatgctttaggcaacacttccatgaaagtgttrcaaattcaaaattgttaataaatatgaaatggaaagaatcctcctataatggcctcaagcccttagtaaagaattaggatccaagaaacaccattagttcccagcttttcaaaacctcactaaggctggaatatgctcaaaccaccacttatcttagaccactgctaatttcaaaaaagttttaagttttgaatgtacat

SEQ ID NO: 62 - DNA, 401 bp, Homo sapiens
catggtgtgcagtatggttgtttcccatggaaatatgttgtacttctgaaagccatggaagcatgaaaaacagattgaattataattttatctgacttttattgtcttttgatctttttaaaaatcatttcttgcttatggaaatttcccatataatttgctgcttcccatttgctgtgggacagaaacattcctctttcrggggaaaacaataacccatcctgttgcttagccatactcaatcttagaaatgggcataccagcttgtggtgctccctagagagaatgagactcagggatgagacccacaaataccttggaagcagatttgaggcctgttggacagaaattctgggattgcagtgcccaaaccctagaaggggaaccgtggactgtaggtg

SEQ ID NO: 63 - DNA, 401 bp, Homo sapiens
tcactctctccccactacacaccactggcagcccctcaaccctggaaaaaggaaattatgcacactatttgcctatacagtctttcacatttaggatgaaatattgagttccaaaacctgctctacatttacttttctagaataacggatacatttcaatcctggtaactttttgctgttcaagaattagaagttgaggawagaaggtttaggaaactctcaaggcccggtttatgctgtagaaaaaaagaatttctgcataagtaaactgcaattataaattttgctccaaatatgaataattcctccaaggagaggttcataacctcattcatctttgtattcctggtgcctagcagggaagaaacctgccataaatgttgaaatgaaagtagcaataa

SEQ ID NO: 64 - DNA, 401 bp, Homo sapiens
atgaatatgtagatatatgagttgtgtagcacacatacacatcatggcacctctgcacttagacatggatgtctatgcatagacatggatgtgcaggaggtgaatggcacttcagaggacaggttcctgtcagcctctttggattcacgtcccagctctacaactttcagcctgggtgatctggagcaagttactaaatcrttatgtgtttttattgcttcacctataaaatggcacctgcttcatagagtgggcacaagtattaaattagattttatacgtaagcattcagcacagtgcctggtaaactgtcaaaaaatggtggccgtttacattttttctgcataaaagttttgaaggacttcagttaattcagaacataaaagtgggtcatgaaataa

SEQ ID NO: 65 - DNA, 401 bp, Homo sapiens
tatatactctttagtacagatatactaaatcccatttatatgtaattcactgctgtactttagatcaaaagtcaaggaaagattaataacagccatcaacaatattaatgttgttcttgaaaaatgcagtcttaaagagcatatgaaatatctttaagactaacggaaaagaagcatgcagcttaggaaaaaatagagcakatataagtcccactactataaaatcatcaatgcgatttagaagaaaagtcattcccacatttgaagtgctaacgaatactaatctttattagcgctaatttagtttttgattgtgttatgtatacttgtttttaatgtactagaattaaccagtattaactccagacaatgtaattataagccaagtgacttggttcatt

SEQ ID NO: 66 - DNA, 401 bp, Homo sapiens
tttatatgtaattcactgctgtactttagatcaaaagtcaaggaaagattaataacagccatcaacaatattaatgttgttcttgaaaaatgcagtcttaaagagcatatgaaatatctttaagactaacggaaaagaagcatgcagcttaggaaaaaatagagcagatataagtcccactactataaaatcatcaatgcratttagaagaaaagtcattcccacatttgaagtgctaacgaatactaatctttattagcgctaatttagtttttgattgtgttatgtatacttgtttttaatgtactagaattaaccagtattaactccagacaatgtaattataagccaagtgacttggttcatttcaaacttttaaaaaattatctttttttccagct

SEQ ID NO: 67 - DNA, 401 bp, Homo sapiens
ttatatgtaattcactgctgtactttagatcaaaagtcaaggaaagattaataacagccatcaacaatattaatgttgttcttgaaaaatgcagtcttaaagagcatatgaaatatctttaagactaacggaaaagaagcatgcagcttaggaaaaaatagagcagatataagtcccactactataaaatcatcaatgcgrtttagaagaaaagtcattcccacatttgaagtgctaacgaatactaatctttattagcgctaatttagtttttgattgtgttatgtatacttgtttttaatgtactagaattaaccagtattaactccagacaatgtaattataagccaagtgacttggttcatttcaaacttttaaaaaattatctttttttccagcta

SEQ ID NO: 68 - DNA, 401 bp, Homo sapiens
aagaatatgatgttatttctcaaaggtacaatctagctgaaatcatatacaagtaagtaggtgtggacttttactgttgagctaaggtttatgtttatatatgttttattctttaagctaaacaaacattcagataacattctatgcattttttgaagcatagggttagtaatgaggacttagattttttaattaaacaaytcagtaactatataaaaagaaaaggagtcccttatgaataaatattaaaattaaaagaaataggcaactataaaagtaagtatttttaataatggcattgattttagtaagaaatcaattaggctgggctggaaagaaaaactggcttaatataaagtagttttaatatgtcaaatattcttcttaaaattgtggccctg

SEQ ID NO: 69 - DNA, 401 bp, Homo sapiens
gcctttctgggggaaaatgcagaggtcaaagagatgatgactacatggactctccagaaaggaatccccctgctggtggttaaacaagacgggtgttcactccgactgcaacaggagcgcttcctccagggggttttccaggaagaccctgaatggagggccctgcaggagaggtggctgcttttcttctttaggtctagyttacctcatctcagtttcctcgttatttccttagctttctctcagctcatctggcaactttgtaggatgctagttccatataagaatcaaaggcctaaagtagacttgataagatttaaagagcttcatataacccggacttctttgttcaggagcaccattcttagtgattccatcagccttgagaacttcagttcttg

SEQ ID NO: 70 - DNA, 401 bp, Homo sapiens
ttgagcctcaagagattcaaaaaatagtttcacctgtaaaatattgaagaaaaaaattactaacactttgtcttggccgagtgtggtggttcacacctgtaatcccagcacttcgggaggccaaggtgggcagatcaattgagctcaggagttcaatacgagcctaggcaacatgccagaacccctgactctacaaaaaayaacaacaacaacaacaaaaattagtcggacatgatggtgcatgcctgtagtcccagctacttggaaggctgaggcgggaggattgcttgaacccagcaggtggaggttacaatgagccaagatggtgccactgcactccagcctggctgacagagcgaggcagtttcaaaaataaagaaacaataaaaccacttcgtctt

SEQ ID NO: 71 - DNA, 401 bp, Homo sapiens
atgtagcattgtttccaggttctcttaaaggttttctttttatctttagttttaagcagttttgaccatgatgtgcttaagacattattatttgtgtgtgtgtgtgtgtgtgtgtgtgtgtgtgtgtgtgtatttatctttttgggggtttgctgaacattttggatctggtaagtggtgtttttcatcaaacttaataawattttaactattatttcttcagatattttcttctgctgcattctcacactccttctgtggctcctgttagatacatgttagactgtctgatactgtcccccagatccctgatgctgtgtttatttttcttcaatcctttgtctcattgttcttcaaattggtaatttctaatgatctgtcaagtttatggactctttctt

SEQ ID NO: 72 - DNA, 401 bp, Homo sapiens
ttgaaaggggtcaggaaagagactccagtctcaacctccttttcactggcttttcctgccatgtattcaccctactatatcctgtatatatccctcaattcaagtaatttgcagagagcagccctgggatagccatccctaatccagttgcctggattacccttccctgagataccagtccgagtcttctgttccccaagscttgtttctggcatccaaggagatggaagtttctgtccctctgtctttggattgtctcctctctcttgagttatcatagtcacctgtcatttcagctcgcctttctgggggaaaatgcagaggtcaaagagatgatgactacatggactctccagaaaggaatccccctgctggtggttaaacaagacgggtgttcactc

SEQ ID NO: 73 - DNA, 401 bp, Homo sapiens
caacacggaagaatctatcatttggtgtgcatactggcagtagagggtgggagttaaaaagaaaatttggccagcaattaccagatcattttaggccagcagtgtaaattcctgtgttattttttgtcacatcatgcttataatcatctcaaaagataaagtaatcatcattactctgtgtttataagtgagaaaactgayactaagggacagatttgcccaaagtcaccaagtcagtgagaaaatcagtacttaaaatttgtcttctaagtccaatagttattcaattatatcacagctagttcctagttttaagaaaagtcccccatcaatcttcccctaaaggtcctagattttgaccaactctcttctgacaccaaagggccctgtagtattaaaat

SEQ ID NO: 74 - DNA, 401 bp, Homo sapiens
tttttcgcctcttgccctcaactccaatgcattttccttacataaattaaaagggaccatcaattggcatacagttccaggcttaaaaaaattaaaagctatatccagggacttatttcgatagttcctgagtcttactctgctattattgtgcaggttctatatactctttagtacagatatactaaatcccatttataygtaattcactgctgtactttagatcaaaagtcaaggaaagattaataacagccatcaacaatattaatgttgttcttgaaaaatgcagtcttaaagagcatatgaaatatctttaagactaacggaaaagaagcatgcagcttaggaaaaaatagagcagatataagtcccactactataaaatcatcaatgcgatttag

SEQ ID NO: 75 - DNA, 401 bp, Homo sapiens
ccggagggtgaggcatgagaagctgaggcatgagaatcacttgaacccgggaggcggaggttgtgatgaaccaagatcacaccactgcactccagcctgggcgacagagcaagactccatctcaaaaaaaaaaaaaaaaaaaaaaaagacgtggttttgtataagaagtaaaaatagtaaacaaacgaaatgtttcagaakcctacctaggagcaaaagaataataatgaagtttgttttgtttcaacggatactgttttacatttgacttcatagagcctttttgagggaatatgatatcacaatttcacaaccaaaacccattatgtttttatctttaacacccgcctatcctcccacacagaacttcctctttagtttaagaatatgacagtttaagt

SEQ ID NO: 76 - DNA, 401 bp, Homo sapiens
gacagagtgagactctgtcttaaaacaaaacaaaacaaacaaacaaacaaaaaacatataaagatgctctttactatccatttccatcacccaccgtcagtggtccagacacactttctccatgcttccgcttaagcttctcagcaccaagtattgtgttgcttctgtctctcatccctctccatttccctctcccttgcyatgtgtgtgtgcatgtatgtatatttgtagacatcagtttagctcccctccaacacggaagaatctatcatttggtgtgcatactggcagtagagggtgggagttaaaaagaaaatttggccagcaattaccagatcattttaggccagcagtgtaaattcctgtgttattttttgtcacatcatgcttataatcatctc

SEQ ID NO: 77 - DNA, 401 bp, Homo sapiens
ctgaggcaggagaatggtgtgaaccccggggggcggagcctgcagtgagcccagatcgcgccactgcactccagcttgggcgacagtgagactccacctcaaaaaaaaaaaaaaaagaaaagaaaagaaaaaaatgccttcaacttggtgataaaaaagcatcaagtaatcatctttaaaaaaaaaatcttatagcaccartagtggccactaaatgatagaatttatataaaccagtagttttgtatttcagaaagcttttatatttgttgattacatcaactttctattttcacctcagagtaggttaaaattttatgcttattttcttgctaacattttatcatcagtaggagataaaacacaaataatttgcagagtaaaagcacataaaatatttt

SEQ ID NO: 78 - DNA, 401 bp, Homo sapiens
ccaccacccctggctaatttttttttttttttgtatttttattagagatggggtttcactgtcttagccaggatggtcttgatttcctcacttcgtgatctgcctgcctcagcctcccaaagtgctgggattacaggcatgagccaccgcgcccggcctcttttttgactttttaacaataatcattccggctggtatgaratggtatttcattgtggctttaatttgcatttttctgatgattagtgatgctgaacatttattcatgttcgttggccacttacatgtcttcttttgagaagtgtctgtgagacggcatattctataagcagttgagaatatcagagtactaagaaaataattctggaataagaattataaggcctctcacagctgtaatc

SEQ ID NO: 79 - DNA, 401 bp, Homo sapiens
actgaattaaataagatgtttataccattgagtcatccattaaaaactaaaacataaataaaagtataccacagttatgaaacaagaaagctaaataaacaggctattatatttttaaaaagttagctgagataatatactaatttccttaatatactcctgccccacaacctgggaccctgccctgggcttgagagggcmctgttttggcattcctctgactatgtctgtccccaccaggcgaggagtcagtaggatcaaagggatgtgcccacctacagtccacggttccccttctagggtttgggcactgcaatcccagaatttctgtccaacaggcctcaaatctgcttccaaggtatttgtgggtctcatccctgagtctcattctctctagggag

SEQ ID NO: 80 - DNA, 401 bp, Homo sapiens
ttttttaatgtgatagcacctagcatatgttgatcttataatagtgattaataagcagttaatgattgattaaagaacttatggtctgtctttgggattcatgtagataataggaaaggcaaagcagaaaaattcagttaattcagatgattctaataattattaaaatattttaaaatttccaactgcaaagaaaataawtttttatagaaccattagacccagagaactcatacctgtaattagaagaaccctaagtcattgtagacagaagagatcctttcttttttacaagcacttgtgtcccagggacagtaataatattgtttaatatttctgcagcagtttacagtttaaagacactttcatggccgggtacaatggctcacgcctgtaatccc

SEQ ID NO: 81 - DNA, 401 bp, Homo sapiens
ccccactgctagctaaaaatatctcagcgcaaaatgtttttgagtggttactactgcattggcatcccttaagctctgaaaaatgccaaaataagtatgctgttaggtttggaaatacagtatatgtttttctttttccttacctctgggaagttatagaatcactacaggaaagagaaaagaaagtcatcacaggggaaraaaggaaaactttttatttaaacaaagagtcatgctaatcccctgaatatatatatacataaatttatatttatttattttagacaaagtctcactctgttgcccaggctggagtatagtggcacaatctcagctcactgcaacctccacctctctggttcaaccaattcctctgcctcagcctcccaagtagctgggat

SEQ ID NO: 82 - DNA, 401 bp, Homo sapiens
aagcctgaaatcagttttagaaaaaaaaaaacttaaaaaaaaaccttttaaatctattattctcttctttttgtttctgtttcaatgggttgtatgagtgaagctaaaatgtaaacatcctactgccctatacaaaatagaatactattatttcatctttatgctagttacaagaaagataatcttaacctgcagtaaccyacctacagtagatataagtgttcaacatgttgaatatacctatgaaaatattctaggtaaacttatttatgctcacaatcaaaaatatgtgattaaatattgttggttttttctaaactccaagattgctagtatgaattttaatgaagaatttctttacatagattaattgattacttcattcatttgtggatttgaaa

SEQ ID NO: 83 - DNA, 401 bp, Homo sapiens
gttcatacttgctttcctttgtacattgttatctccagtgtttgaaattctcttatccccagttaatttgtaattgtcttgaacactacttgataaagtgttcaactttagtatttcagaaaggaaatatactctccagtgaaagaataagcttcaagttattcagcatagatagaacaggttataagtattatcaagcarcagccttctcaaagggatttttatgtgagatagttatgattggatctcttaacaacagtttaaggctttttcgccttagtgtgttatgatctaatttttatccaaaagtggtgggtctctcttgtatgaatagtagggataacatcaaactttattccctggtcccttatgcttctcttccaaaatcttctgttaactac

SEQ ID NO: 84 - DNA, 401 bp, Homo sapiens
tctagaaagtaacagaataattgtgaatgtttataaatagctatatattgtcagtcaaccatatttattctgcttgctatgttgtcatggtctatagaaggcagcacttccctttttcctctaacaccacactaggatgtcacgctggggtggccccaggggctcactatttgacctgactttctttggtttctcttctaygactaatcctacacttttatgttcccttgatctaaaatctttaaaatgttgtaatttctaccatataacacctaatctctggcaaattttaaggttgtcaactgtattccccaatgtgtatatatttgttgagcaaaactaatcttctctcttataagaagaaatcttgctcaatctctagatcattgtgcctatatttc

SEQ ID NO: 85 - DNA, 401 bp, Homo sapiens
tataattgtaatccaatattttaaaatgtggagaactttacctaaaaatcctgacctctggaatctcttaaaaatgatatttggcaactgtgggtctttatcctgtgggccacaggatagctataaaggagagtgcagtgggccaaactctctttaggccaggcctactttctcccaagcaccaaagtcccaaatggcctstttcattcacttagtgtggactctctaagtatttgagctttcgaccccacatttaaaatgtatttatgttattagctgctacaccaaataccggtgtaatctcttaaggaaaagtacaaatatagtcttaacttttataattttaaatgggttgttatgaaatctattcattacttacactggagaaattagtatgacgt

SEQ ID NO: 86 - DNA, 401 bp, Homo sapiens
aactgattctagccactttaccagttagcaagactagtttattcattcattcagtaaatacctacggagaacctactgcgttcctggcaccatgccatatgttagaaatacaaagatgtatataatatagccactgcctcaatacaaatgatggaggtcaaccagtagacaaataaatacagtaaagcaggtgctaagatmaatgtctttgccagggacagaggggattccaggaggaaataatcagttttgccaggacagtgttcatttcctgagccttgtaaatagtactcaggtatgctgaatatcaggtagtaggagcaaaggcagagtgatacaacctggcatgttcagaaaatggcaggtaatcttggatggctaaagcttaggtgtaggcaaga

SEQ ID NO: 87 - DNA, 401 bp, Homo sapiens
ttcattatcatgtctggatgaattatttcatatagctcttttaataaatgcctgcatccatggctaatgtgcacgttcagccaactaatgatgcctacattgcagtgctcttttgttcttgttttgtagagttgtttagaaagtgattttacatctggtggagtttgtcattcggatcccaagatgacaagtaacatggtraggataaagagagtcacagagtagaagagatctgtggaatagcctgacctagagtgagtatgacatacagagtagcccacctgtcccttttaaaagctggagagaaagagagcccccacgattttctctaaaacaaaactgaaggggaaatgcttggggtatttagggggacaatgctgttgctactatatttttgttgt

SEQ ID NO: 88 - DNA, 401 bp, Homo sapiens
tttgggctagggagtttcaaagttgactccactgaactatttggatacaaatggtattatttatatgctttgagaaacatttgattacacttggtttgaggcaactgagacatctgcatggaagaacaaacattggatgaaaatgaataccaactttttcagaagatgggtccaattttctcttacaaaatcccatgctarttgctgcccctttggacgtctggcaatcgcatgaaggagagctgccaagttctgtgtcttgataacctttccttccattcctagttcaattaacctgaagaaagaaaaataatttgtttctaaatagtaggtattatgtacatggacattaactcaagccaccaatatattaaaagaatagaacagaaagaggcatgata

SEQ ID NO: 89 - DNA, 401 bp, Homo sapiens
gccacatatgtgtgatgcactatacaaggcatcttgggattcttctgggctgaggagagagccaagaagggatggtgggagggggctgatgactgcatttaattcaactcggacttgatgccagtggtttcttgcctggactgaatgagagaaggctgtttcccattcccttattctcatgtctctcccttactcctggayaggataatttgggctagggagtttcaaagttgactccactgaactatttggatacaaatggtattatttatatgctttgagaaacatttgattacacttggtttgaggcaactgagacatctgcatggaagaacaaacattggatgaaaatgaataccaactttttcagaagatgggtccaattttctcttacaaaatcc

SEQ ID NO: 90 - DNA, 401 bp, Homo sapiens
ccagtacaaaactgcattaacaaatgaggccacatatgtgtgatgcactatacaaggcatcttgggattcttctgggctgaggagagagccaagaagggatggtgggagggggctgatgactgcatttaattcaactcggacttgatgccagtggtttcttgcctggactgaatgagagaaggctgtttcccattcccttwttctcatgtctctcccttactcctggacaggataatttgggctagggagtttcaaagttgactccactgaactatttggatacaaatggtattatttatatgctttgagaaacatttgattacacttggtttgaggcaactgagacatctgcatggaagaacaaacattggatgaaaatgaataccaactttttcagaag

SEQ ID NO: 91 - DNA, 401 bp, Homo sapiens
tcttaccaaaattcctgagattttcccccagtacaaaactgcattaacaaatgaggccacatatgtgtgatgcactatacaaggcatcttgggattcttctgggctgaggagagagccaagaagggatggtgggagggggctgatgactgcatttaattcaactcggacttgatgccagtggtttcttgcctggactgaakgagagaaggctgtttcccattcccttattctcatgtctctcccttactcctggacaggataatttgggctagggagtttcaaagttgactccactgaactatttggatacaaatggtattatttatatgctttgagaaacatttgattacacttggtttgaggcaactgagacatctgcatggaagaacaaacattggat

SEQ ID NO: 92 - DNA, 401 bp, Homo sapiens
tgatagctaatttctttgggggagccattgaacatatttgagcatttcttagtttaaagcaagcttgacagagggcggatccaataagttcttcatggcttcccacataggtctagaagaaacctatgttttatttgaattgtgtttgtggtcatcattttggaaagctagttgacaagtgttaaagtatatcatagagaygaactcaagtgggttcctctctatgatatacttggattatgatacaatgggttaacatgatttgaaagttacagatgagcactgaggaaatggattgtaacagaagattcagggtgtttgtaattttcaagactgacttttgctttaatacttcacagaacctcttatccttattgacaacatgcttaatggtatcttaa

SEQ ID NO: 93 - DNA, 401 bp, Homo sapiens
aaacttgctttgatctcttccctctttctctttctatgtgatttaaatgagcactgaggaattcagttagctcaggaaaaaataatttgttcctcagagatgattcttgagtgtagaaaataaaatatttatgacatgccccaacagtgtggatcatttctctattcttttatcaggtttaacgttaacatatctgcatcraactctttcccaggctgatcagaaagggcacacactggaggctgggacggaagccaggtagagggtccaggaaagagatggggagaaaaagaaggaacacagtgactgctctgttcaaaataggggtccacatgtccaagatgctgtggctccctgtggcggacatcaacgctctcatccattatgctcctcttctgtgg

SEQ ID NO: 94 - DNA, 401 bp, Homo sapiens
catccaaggagatggaagtttctgtccctctgtctttggattgtctcctctctcttgagttatcatagtcacctgtcatttcagctcgcctttctgggggaaaatgcagaggtcaaagagatgatgactacatggactctccagaaaggaatccccctgctggtggttaaacaagacgggtgttcactccgactgcaacargagcgcttcctccagggggttttccaggaagaccctgaatggagggccctgcaggagaggtggctgcttttcttctttaggtctagcttacctcatctcagtttcctcgttatttccttagctttctctcagctcatctggcaactttgtaggatgctagttccatataagaatcaaaggcctaaagtagacttgataag

SEQ ID NO: 95 - DNA, 401 bp, Homo sapiens
gggaaaggggccattatctggtattttacttaaaagcacagaagttgaattgatgccagtgttggaaattattgcattttaagaaaatagaaatatgtaatatttttatgctttcaatcaacaaaatgagatttggcatttttgtgctttggggatctcaaaagcagggctttttgttttcaacagagtgttggggtaaargcaatggaggtaagagaggctacagaatactaggagaggccattgcccccctaggaggtcatcgattgtccttcagagtatgaggcttgcctctaactcacctgccataagtcataggcatggttatgaaatactccagttttcaagtactgattattccttttcctttctgtaggttaagacaattgaacttgaaggag

SEQ ID NO: 96 - DNA, 401 bp, Homo sapiens
gaaaggggccattatctggtattttacttaaaagcacagaagttgaattgatgccagtgttggaaattattgcattttaagaaaatagaaatatgtaatatttttatgctttcaatcaacaaaatgagatttggcatttttgtgctttggggatctcaaaagcagggctttttgttttcaacagagtgttggggtaaaagyaatggaggtaagagaggctacagaatactaggagaggccattgcccccctaggaggtcatcgattgtccttcagagtatgaggcttgcctctaactcacctgccataagtcataggcatggttatgaaatactccagttttcaagtactgattattccttttcctttctgtaggttaagacaattgaacttgaaggaggt

SEQ ID NO: 97 - DNA, 401 bp, Homo sapiens
cgagttatgcttggtatagctattaggtaattcaattaacttgcaaaatagatgaagaaagcaattctgagaagatcagctgaaatcactggaaaaactcaaaaaggcaagccactaaaattgttgttgaattaggtaggagacaactctaaaagactggggaaaaaaattgaaagtctagacaaattttgcactcggacygcttcacaggtgtccacattttcatttcactttataataggttatagggtcaaaaacttgctgaagcaaaacacacaggggtgagtttctgttttaatattgttccatttcctttctctactttgccctgaaggcaggctttggtcacagagtggtgtggaaatgccaacaggtaaaattccaatgagtcccatatttct

SEQ ID NO: 98 - DNA, 401 bp, Homo sapiens
tcagctcactgcaacctccgcctcctgggtccaagtgattcttctgcctcagcctcccaagtagctgggactacaggtgcgtgccaccacattctgctaatttttgtatttttattagagacagggtttcaccatattggccaggctggtctcgaactcctgaccccatgatctacctgccttggcctcccaaagtggtgkgattacaggtgtgagccactgtgcctggccaagtgtcaggttttaatcctgtcccttccatttacttgctatatggcattggacaaacaacttttttaaaaactaaaatgagaacttcaaatcagattatatctaagtttactttcaattccacaatttgaacatttattttgaaattgttaaaaacagaaagtcacaaa

SEQ ID NO: 99 - DNA, 401 bp, Homo sapiens
acattttaatgtatataaatatttgctacattctgtgtgttatataatgtggtacccagtcctctgctgggacatggatgtacataatgaaacatggaggtccagacgtatgataactctcctgtttcccttccctcattgcctacaggggcaatagtttcatatcttgggttttttattgtttaatttttttttatgggraggggttctttgggtgggtaatagtcagggggaaaggacagtgtctatactttttaaagatgtatataaatgtttcatgttattggttttgtacctagtcctttgcatggatatataggtacctaatgaaaatcgaggatcagtgtatgacaaatctcccatcctcccctttccttattgcctgtgtcggcaataggaag

SEQ ID NO: 100 - DNA, 401 bp, Homo sapiens
atcattactctgtgtttataagtgagaaaactgatactaagggacagatttgcccaaagtcaccaagtcagtgagaaaatcagtacttaaaatttgtcttctaagtccaatagttattcaattatatcacagctagttcctagttttaagaaaagtcccccatcaatcttcccctaaaggtcctagattttgaccaactcycttctgacaccaaagggccctgtagtattaaaataataaattactgaaaatatcttgcccaccattgtgtcacataaagtcaattctaatacatgtcaatagcaacttgagaatgagaagaattagttgctgttatttttcataagatcatttaaaggcatttgagagccttagcacattcttcattttttctcatttgc

SEQ ID NO: 101 - DNA, 401 bp, Homo sapiens
gtatgctgttaggtttggaaatacagtatatgtttttctttttccttacctctgggaagttatagaatcactacaggaaagagaaaagaaagtcatcacaggggaagaaaggaaaactttttatttaaacaaagagtcatgctaatcccctgaatatatatatacataaatttatatttatttattttagacaaagtctcrctctgttgcccaggctggagtatagtggcacaatctcagctcactgcaacctccacctctctggttcaaccaattcctctgcctcagcctcccaagtagctgggattacaggcacacaccaccatgcccggctaatttttttgtattttcagtagagatggggtttcaccatgtaggccagactgacctcaggcaattcg

SEQ ID NO: 102 - DNA, 401 bp, Homo sapiens
acaggcatgatccacggcgcctggccctaaattgtgttttctaaaggaaggttcagcatcatccagtgatcagaaacccatactagcagtgcagcagccagaggttttgttctccattcactacacctctattgatattaaattgttctgttgaaatatttaaagcttccctaaagacagatatttccctcgtaaaccacycctcctggattctactgttatttgagggttttgtttgtttgtttgtttgattttgtttgtttgcttgtttttgaaaggggtcaggaaagagactccagtctcaacctccttttcactggcttttcctgccatgtattcaccctactatatcctgtatatatccctcaattcaagtaatttgcagagagcagccctgggat

SEQ ID NO: 103 - DNA, 401 bp, Homo sapiens
agaataaattgtctgtgaaaatactgaaaacatacaaaggacatttttttctcagttttaaaactgtattccgctttaaaaactgttttctaggccgggcgcggtggctcacgcctgtaatcccagcactttgggaggccgaggcgggcggatcacagggtcaggagatcgagaccatcctggctaacacggtgaaacccygtcgctgctaaaaatacaaaaaattagccgggcgcggtggcaggctcttgtagtcccagctactcgggaggctgaggcaggagaatggcacgaacccgggaggcggagcttgcagtgagccgagatcaggccactgcactccggcctgggcgacagagagagactccgtctcaaaaaaacaaaacaaaaacaaaaaaaac

SEQ ID NO: 104 - DNA, 401 bp, Homo sapiens
agctggagtgtcacgatcgtagctcactgtaactttcagtttctgggttcaggtgatcctcctgcttcagcttcccacgtagttgtgactgaagatgtgcaccacaatggctgtctaatttttattttttaatttttgtagagatgggggtctcactatgttttccacactggtctcaagctcccgtcctgccgccttggsctcacaaagggctagaattacaggtgtgagccaccacgccaggtcctggctgtttggattttaagaccaagggaaacatactatttgttgacccctcggtctcactgaggacctggaaagaaataacagcaacagtgctggccttgcaaatccaaacctaaaacgtgctgtctaaaaaaagaaacttcaggcggggcgta

SEQ ID NO: 105 - DNA, 401 bp, Homo sapiens
gacatgttttgctgagtgtggttggttacttcaggtacagtatcacattaataggggccacagtctatatccctgtttagtttgttagttaccactcaagagcagacagatattgtccactttatcccaaatcccagccagactttgttgtatgctgtgcttcattcatggggctgtgaactactgattatattctccctwttcctaatgtagaatgctttattctactgccatctttctgtctgcactgtttaattaggcttactgataacaactttaattctgaattttctttctcattcaggttctatttgtaattactaagacttaaagaatagtctggtgaagttactcgaagaattaaggaaggtttgagctaaaatgaactagagaccatctag

SEQ ID NO: 106 - DNA, 401 bp, Homo sapiens
cacatttttgagaagtgatgctaaaaatttttttttaaaaagactcacatatctatagaacaattgttatttgtaagattaaaagatggaatcacaattttatactgtattacaacccacaaatatctcatttgttgcccagacttcccatttttgaagttgaaaaatactttcaactggataccaatctgaacatgaaarcaaaaataattttttaagacaactaagtcctccttgtttgattatgcaccacactgcggtaataaaagtgattcataggacctacattcatatgaaaaaaaaaactatttatgttttcagttctgggacctcaaaatgccaaaataccaacctttagatatactttagaatatatcacaaactagaaagtatatatactt

SEQ ID NO: 107 - DNA, 401 bp, Homo sapiens
actataaaatgaaaatgtagatacagcttctttgggaaatatggttagttattcagcaatgtttatgtttaattttatgtttctgtttaaagcagtgttccctactttttttgttactacatactccagtcagtaaagattttttgagcaagaacttcccaatatacatatttttatttataaattatatgggctgggcgyggtggctcacacctgtaatcccagaactttgggaggccgaggtgggtggatcacctgaggtcaggagtttgagacctgcctgaccagcatggagaaaccctatctgtactaaaatacaaaaaaattagctgggcatggtggtgcatgcctgtaatcccagctactcaggaggctgaggcaggagagtcacttgaacccgg

SEQ ID NO: 108 - DNA, 401 bp, Homo sapiens
tatgtttctgtttaaagcagtgttccctactttttttgttactacatactccagtcagtaaagattttttgagcaagaacttcccaatatacatatttttatttataaattatatgggctgggcgcggtggctcacacctgtaatcccagaactttgggaggccgaggtgggtggatcacctgaggtcaggagtttgagayctgcctgaccagcatggagaaaccctatctgtactaaaatacaaaaaaattagctgggcatggtggtgcatgcctgtaatcccagctactcaggaggctgaggcaggagagtcacttgaacccgggaggtggaggttgcagtgagccaacatggcgccattgcactccagcctgggcaacgagaatgaaactccgtctca

SEQ ID NO: 109 - DNA, 401 bp, Homo sapiens
ataaataatgtatttatttaactttttatactatatatcatttatgtttataaatttataacaatataaaatttaaaattagataagaaataatagacactctaatgtattgcacttcttgcacacttcctcatcccattttggataccactgtgtttaaacattgtgtttagtggggcaatgttacttggttgaactctyattcacggccacagaatgcatccttcaacccaatgttttatatatggaaaacttgtactacaggtcaaagtgattcatgttgataaagcagagcacagttacagctcagaaaaaaatatggttccaagtctaggctctgcacatggttgggcaggggcatcatttcctgtttaaaatgaaatccacagcattgtagatta

SEQ ID NO: 110 - DNA, 401 bp, Homo sapiens
tgatttttattatcttgaggacattaaccagtttttattctaagtgggcatatgactttttaatcccaaatgtcaacggatcaataagaactgttattgatgtctcagaaaaaacacaacacagagcattgaggagggagccagaaatttgcatttgtcacaaagtgaccatatggtagagattgtactagtctccatacmtcttatttaactgtcaaaagctaccctctgagatagttattatcctgatttttttaagcggaaataaatctcagaaaagttaagtaacttgcacaagatgacataatttgccatttgcagatgggatttaaccctatgattctaatgctttggctacttcctctatactatatgtacttaaataccccaagtgacatttg

SEQ ID NO: 111 - DNA, 401 bp, Homo sapiens
attctaatgctttggctacttcctctatactatatgtacttaaataccccaagtgacatttgaataatataataaagatcaaataattataatacatattgttttcattttagtgtattttgctgaacaactttataacaattggtaacaaacacattgtaagcttcttgaaggtaggccacatggttgttttgttcaccwctttatccttagctcctacagcaatacctggctggcataaaggaaatgtgcaactagttacatttcaaatccacaaatgaatgaagtaatcaattaatctatgtaaagaaattcttcattaaaattcatactagcaatcttggagtttagaaaaaaccaacaatatttaatcacatatttttgattgtgagcataaataa

SEQ ID NO: 112 - DNA, 401 bp, Homo sapiens
tttgcatttttagtgcagacagggcttcactatgttggccaggctggtcttgaactcctgacctcagtgattcaccctccttggcctcccaaagtgctgggattataggcgtgggccactgcatccagccagcaccggaataatggacaccattacatttcatggtagagattgtactagtctccatacatagcacttacratgtgagagcatgaaacagggtttgtaatgcccagcatgtttttttttctatcctcactaaaaggttttagacctaatgtcttgcttgatcaaagacttttacatcaaagagaaaagaacaaagggtaggacagaagtctacatctactttaattttccactggattccatccactgggagaagagttcagcagctttct

SEQ ID NO: 113 - DNA, 401 bp, Homo sapiens
gtccatcatgtggtaaaacgattccaagtaactcagaccttcgagaagtgcggggctgcttgtttcatgttggaggtagtaagtcatgtcaagagctttgtctagggtcagtctccctgcactgaagtataaaacaaatgtcagtggtttgtgcatatcttatagtttttaatatttttaacattaaaacaaatatgaaakagagaacaataacagaagtaggtcattatagctgggccattcagtttaaatcttagctctgccactgactagctgagtaatcttggacaaattaccaaacctctctggacctattgccttctttatacaatggggataataatactatcttcctcataggcttgttgtgaggattaatgacctaatattttaaaaagcat

SEQ ID NO: 114 - DNA, 401 bp, Homo sapiens
ttttccaagtcttgcattgttaatctatcttctcttcaatctactcagagcttctcctttgagcaggcaaacctccttcaactagcaatagcttgcttcttagctcacctttcatgcattcactcatatgtaggatggtgtttttcctaattttggcctccaatgagctatgcctccaagatgttcctgagacccacgggrctgaggattaagcctcacttcctcttttctttctaactagaggattttagtgtaccgagaaactttgtctcagagatgtcctgctggtcactttcactcaatttgacctaaataagctcctgaaagaaaattatatgtcacattgagtaaagtgagtgcatcacagtaattctcagaatagggaaagcttgcaatgcaca

SEQ ID NO: 115 - DNA, 401 bp, Homo sapiens
ttcactcaatttgacctaaataagctcctgaaagaaaattatatgtcacattgagtaaagtgagtgcatcacagtaattctcagaatagggaaagcttgcaatgcacaatatacttcctggtcctcaattccctccagccattggtgatcttgtgacctgattcattccacacattattctgttcatgatacatagaaaargaaagaaaacactttgtccttccaggaaagtctagagaggaatgaatgcaaacagccatttattacttcatttccctacaaacgtcatactaatttctccagtgtaagtaatgaatagatttcataacaacccatttaaaattataaaagttaagactatatttgtacttttccttaagagattacaccggtatttggtg

SEQ ID NO: 116 - DNA, 401 bp, Homo sapiens
ggaatttggcttaatttgatgatgtccttgtctcaaggtttagtcactagtcattgaaatatgtatgtgtaaataggtgatccatttgttcattttagtaaagaaggactccaggttaagcatgactttgtgacggaaaacctctcaaattttattaaagtgtttagaagaaaattaaaattatactatgtatttttaatrtggcatatattatactagagggtaaaattacattataatattctctcaacaactctgtgaggtttagtctttattcaacataagatgaaaaaattgaagctcaggatgagtgtgtacattttcttaaggtcacacatctaataagtgagagagtgaggacttgaatccagaagcaatcaattttaaagtatgtgcttttt

SEQ ID NO: 117 - DNA, 401 bp, Homo sapiens
aaattatactatgtatttttaatgtggcatatattatactagagggtaaaattacattataatattctctcaacaactctgtgaggtttagtctttattcaacataagatgaaaaaattgaagctcaggatgagtgtgtacattttcttaaggtcacacatctaataagtgagagagtgaggacttgaatccagaagcaaycaattttaaagtatgtgcttttttccactgaacattttttgccttatccataacctgtaaaaatagattagtgggtattataagacataagatagatttctgttatttcttgatgtaaataatctgtctctaaatgataaaagcgcaagagaacttcccactgaatgaaaaatccagattttcttactaaaagagttatt

SEQ ID NO: 118 - DNA, 401 bp, Homo sapiens
cctgccttagcctcacgaatagctgggattacaggcaagcaccaccatgccaagctaatgtttgtatttctagtacagacggggttccacgaattggccaggctggtctcaaactcctgacctgaagtgatctacccaccttggtgtcccaaagtcttgggattacaggcgtgagccattgtacccggccatgaaagtgtytttaaactgtaaactgctgcagaaatattaaacaatattattactgtccctgggacacaagtgcttgtaaaaaagaaaggatctcttctgtctacaatgacttagggttcttctaattacaggtatgagttctctgggtctaatggttctataaaaaattattttctttgcagttggaaattttaaaatattttaataat

SEQ ID NO: 119 - DNA, 401 bp, Homo sapiens
cttaaaatttgccagagattaggtgttatatggtagaaattacaacattttaaagattttagatcaagggaacataaaagtgtaggattagtcgtagaagagaaaccaaagaaagtcaggtcaaatagtgagcccctggggccaccccagcgtgacatcctagtgtggtgttagaggaaaaagggaagtgctgccttctayagaccatgacaacatagcaagcagaataaatatggttgactgacaatatatagctatttataaacattcacaattattctgttactttctagattaaataacagtctatcgttacccaacatatgacttacatttgacagactgctccacaagtcatcattcttagcatttctatagctgaacttctttaagtactgaat

SEQ ID NO: 120 - DNA, 401 bp, Homo sapiens
aataacagtctatcgttacccaacatatgacttacatttgacagactgctccacaagtcatcattcttagcatttctatagctgaacttctttaagtactgaattattcctttctggaatttctcctcacccagaaaatccttgagcatattcaaaatacaagctccctttaaaaaaaaacaaaagagttgaaaaaagagwtaaagaaaatggtagtatggtatgtttttaaaggaagcttaaattttacggaacatgtgtgatgtctgaaaagtgaacaaataaaaagtgaaacaagtagcaggaactggcaccagtgacttaaactgctgattctatagtcattattacacttctgaaagcagagcttccacctgcacctgatatttactaccttgtta

SEQ ID NO: 121 - DNA, 401 bp, Homo sapiens
caaaagagttgaaaaaagagataaagaaaatggtagtatggtatgtttttaaaggaagcttaaattttacggaacatgtgtgatgtctgaaaagtgaacaaataaaaagtgaaacaagtagcaggaactggcaccagtgacttaaactgctgattctatagtcattattacacttctgaaagcagagcttccacctgcacstgatatttactaccttgttataggaaacttcatcaaacatttcctgtatttgagtcggggtttccgctggtttggagatagggcgggatgaattcaatgaatcttttgtaattacttcaaaacacacattcaaaaaatagtcatcctaaacagggagaaaaatgtttagttttagtttctatttgacactgtaaaagcaa

SEQ ID NO: 122 - DNA, 401 bp, Homo sapiens
tgatgtctgaaaagtgaacaaataaaaagtgaaacaagtagcaggaactggcaccagtgacttaaactgctgattctatagtcattattacacttctgaaagcagagcttccacctgcacctgatatttactaccttgttataggaaacttcatcaaacatttcctgtatttgagtcggggtttccgctggtttggagatwgggcgggatgaattcaatgaatcttttgtaattacttcaaaacacacattcaaaaaatagtcatcctaaacagggagaaaaatgtttagttttagtttctatttgacactgtaaaagcaatagaaaacatagtaggtttagtaagatgttcttagaggtaagatttcaatcgatatttcttgggagatgtttcttttctt

SEQ ID NO: 123 - DNA, 401 bp, Homo sapiens
taagaatatataaagctttggcattaagccacaaattcagtacatacacagtaacaagaagagcctaactttgaatccatgtctgtctatagtgtactggactaaatatatatcccaaagacctaattaaccattactaaccaccttgatatgcaaatttgtgtagtgttcagaccactatattcgtttttaaaaaagacrtacctgaagaaatcccttcacacattttggggaagccagcagaccctttggaaattctagaaaagtacacccccaatgatgttgatttcaggttacatgccgagaactctctatagtacctagtagccatgagcattcctgtgcagatgtatacaaacagtgatgttctttcctctcaacccacatacacagttctacat

SEQ ID NO: 124 - DNA, 401 bp, Homo sapiens
gtctaagatttaaaaaatatataaatcaaataaaaaggatgcataaatataattgacatatttaccctttacctatatttgatctgtattatgcattttaaatattactattcttttatgtgcttttatgttttatttatttagtctatatgcctaatactgcacatctagtgtatgtctctaagattaaaatctctagayggaccaaagcttcaacaataagattctaagattcagaagagcctggttatagttacagaacagaaaattataagtctgtagcttctagaaacagtttaagcactattccttttctgacagtcttcagattactttacaagtgggcagcaatcttctgaagggcattcatggaaagggagaggtgtttcctcaatttgaaa

SEQ ID NO: 125 - DNA, 401 bp, Homo sapiens
gctaactggtaaagtggctagaatcagtttatcctgtacttcttatattcaccggttttcaaattgaggaaacacctctccctttccatgaatgcccttcagaagattgctgcccacttgtaaagtaatctgaagactgtcagaaaaggaatagtgcttaaactgtttctagaagctacagacttataattttctgttctktaactataaccaggctcttctgaatcttagaatcttattgttgaagctttggtccgtctagagattttaatcttagagacatacactagatgtgcagtattaggcatatagactaaataaataaaacataaaagcacataaaagaatagtaatatttaaaatgcataatacagatcaaatataggtaaagggtaaatatg

SEQ ID NO: 126 - DNA, 401 bp, Homo sapiens
agaagaaaattgtacagagagaaaagggtagcaaagagagaagagagatcctaactaataaaaaaaaagttagtaactattgtattttttgctaaagttaataatttttatttgtttaacttctaataatattgagtttttacctcctagtggtttggcaacctggtcacaatggaatggtggaatgatatttggcttaakgagggttttgcaaaatacatggaacttatcgctgttaatgctacatatccagagctgcaatttgtaagttcacaattctgtgtatcatactatatggtgtaaagaatcatcaattcactattaaaatttcaagtgaatgttaaacagaaaaactacataatgttgtggtttttgaacatatggcattttgtttgatacac

SEQ ID NO: 127 - DNA, 401 bp, Homo sapiens
ttgaacatatggcattttgtttgatacacgaaacagatcacagaactggatgaaacattgaaggttttagaaaacaatcaacataaatctgtcaccccaaagtctgtaaagagagaaggcaaactaatacaaatgtagaactgtgtatgtgggttgagaggaaagaacatcactgtttgtatacatctgcacaggaatgcycatggctactaggtactatagagagttctcggcatgtaacctgaaatcaacatcattgggggtgtacttttctagaatttccaaagggtctgctggcttccccaaaatgtgtgaagggatttcttcaggtacgtcttttttaaaaacgaatatagtggtctgaacactacacaaatttgcatatcaaggtggttagtaat

SEQ ID NO: 128 - DNA, 401 bp, Homo sapiens
gtgggttgagaggaaagaacatcactgtttgtatacatctgcacaggaatgctcatggctactaggtactatagagagttctcggcatgtaacctgaaatcaacatcattgggggtgtacttttctagaatttccaaagggtctgctggcttccccaaaatgtgtgaagggatttcttcaggtacgtcttttttaaaaackaatatagtggtctgaacactacacaaatttgcatatcaaggtggttagtaatggttaattaggtctttgggatatatatttagtccagtacactatagacagacatggattcaaagttaggctcttcttgttactgtgtatgtactgaatttgtggcttaatgccaaagctttatatattcttatttgtaaaatgcatat

SEQ ID NO: 129 - DNA, 401 bp, Homo sapiens
tttttgaatgtgtgttttgaagtaattacaaaagattcattgaattcatcccgccctatctccaaaccagcggaaaccccgactcaaatacaggaaatgtttgatgaagtttcctataacaaggtagtaaatatcaggtgcaggtggaagctctgctttcagaagtgtaataatgactatagaatcagcagtttaagtcaytggtgccagttcctgctacttgtttcactttttatttgttcacttttcagacatcacacatgttccgtaaaatttaagcttcctttaaaaacataccatactaccattttctttatctcttttttcaactcttttgtttttttttaaagggagcttgtattttgaatatgctcaaggattttctgggtgaggagaaattc

SEQ ID NO: 130 - DNA, 401 bp, Homo sapiens
aatggcctgtttcattcacttagtgtggactctctaagtatttgagctttcgaccccacatttaaaatgtatttatgttattagctgctacaccaaataccggtgtaatctcttaaggaaaagtacaaatatagtcttaacttttataattttaaatgggttgttatgaaatctattcattacttacactggagaaattartatgacgtttgtagggaaatgaagtaataaatggctgtttgcattcattcctctctagactttcctggaaggacaaagtgttttctttctttttctatgtatcatgaacagaataatgtgtggaatgaatcaggtcacaagatcaccaatggctggagggaattgaggaccaggaagtatattgtgcattgcaagctttc

SEQ ID NO: 131 - DNA, 401 bp, Homo sapiens
agaaagaaaagaggaagtgaggcttaatcctcagtcccgtgggtctcaggaacatcttggaggcatagctcattggaggccaaaattaggaaaaacaccatcctacatatgagtgaatgcatgaaaggtgagctaagaagcaagctattgctagttgaaggaggtttgcctgctcaaaggagaagctctgagtagattgargagaagatagattaacaatgcaagacttggaaaaaatggagaatattgacaattcagcaaattaatcttttaggtagttgtgaaatcttttttgctgtttctagcctatccacttagattgtctaaatttagtaggaggaaatttgcagttattgatgcattggtggaaaactaatcatcttttcttcactaagtagaca

SEQ ID NO: 132 - DNA, 401 bp, Homo sapiens
aagcgatcctcccacctcagcctcctgagtagctgggactacaggcacacaccaccatgcccaaccaatttttaaattttttggcagagatggcgtctgcctatgttgtccaggctggtctcaaacttctgggctcaagcaatcctcctgcctcggcttccccaattgctgggattacaggtgtgagccactgcacccagmatggagagagaatttgatgcaagaattgatatttattttagttcggttttcatacattttaaatgtaatttaaagacaggggtcttggataagttgagtggaattgaaatgacaacttcaatttgcctatagaaaaagctatatttgtttcttttagtcccacaccttaaagagaaaaccccacattgggcgcagtggct

SEQ ID NO: 133 - DNA, 401 bp, Homo sapiens
tcctcctgcctcggcttccccaattgctgggattacaggtgtgagccactgcacccagaatggagagagaatttgatgcaagaattgatatttattttagttcggttttcatacattttaaatgtaatttaaagacaggggtcttggataagttgagtggaattgaaatgacaacttcaatttgcctatagaaaaagctayatttgtttcttttagtcccacaccttaaagagaaaaccccacattgggcgcagtggctcacgcctgtaatcccagtacttcgtgaggccaaggcgggtggatcacctgaggtcaggagttcaagaccagcctggccaacatgttgaaaccccgtctctactaaaattataaaaattagctgggcatggtggtgtgtgcct

SEQ ID NO: 134 - DNA, 401 bp, Homo sapiens
gattacaggtgtgagccactgcacccagaatggagagagaatttgatgcaagaattgatatttattttagttcggttttcatacattttaaatgtaatttaaagacaggggtcttggataagttgagtggaattgaaatgacaacttcaatttgcctatagaaaaagctatatttgtttcttttagtcccacaccttaaaragaaaaccccacattgggcgcagtggctcacgcctgtaatcccagtacttcgtgaggccaaggcgggtggatcacctgaggtcaggagttcaagaccagcctggccaacatgttgaaaccccgtctctactaaaattataaaaattagctgggcatggtggtgtgtgccttcccagctacttgggaggctgaggcaggag

SEQ ID NO: 135 - DNA, 401 bp, Homo sapiens
tattaaccttttgctatgtggtagacattattctaaatgctttttaaaatattaggtcattaatcctcacaacaagcctatgaggaagatagtattattatccccattgtataaagaaggcaataggtccagagaggtttggtaatttgtccaagattactcagctagtcagtggcagagctaagatttaaactgaatggyccagctataatgacctacttctgttattgttctctatttcatatttgttttaatgttaaaaatattaaaaactataagatatgcacaaaccactgacatttgttttatacttcagtgcagggagactgaccctagacaaagctcttgacatgacttactacctccaacatgaaacaagcagccccgcacttctcgaaggt

SEQ ID NO: 136 - DNA, 401 bp, Homo sapiens
tagtattattatccccattgtataaagaaggcaataggtccagagaggtttggtaatttgtccaagattactcagctagtcagtggcagagctaagatttaaactgaatggcccagctataatgacctacttctgttattgttctctatttcatatttgttttaatgttaaaaatattaaaaactataagatatgcacaarccactgacatttgttttatacttcagtgcagggagactgaccctagacaaagctcttgacatgacttactacctccaacatgaaacaagcagccccgcacttctcgaaggtctgagttacttggaatcgttttaccacatgatggacagaaggaatatttcagatatctctgaaaacctcaaggtttgtgttgcttttag

SEQ ID NO: 137 - DNA, 401 bp, Homo sapiens
ataagagaaatacgaagatacactgtttggggaaagattgggaaagatgcagaaagtttagagttgagccctttagatgggcaagaactgtgttaaggactaaatttagcctctctgttaaccatctcatattttctgcagcgttaccttcttcagtattttaagccagtgattgacaggcaaagctggagtgacaagggytcagtctgggacaggatgctccgctcggctctcttgaagctggcctgtgacctgaaccatgctccttgcatccagaaagctgctgaactcttctcccagtggatggaatccagtggaaaattaaagtagatgtagacttctgtcctaccctttgttcttttctctttgatgtaaaagtctttgatcaagcaagacattag

SEQ ID NO: 138 - DNA, 401 bp, Homo sapiens
acaggcaaagctggagtgacaagggctcagtctgggacaggatgctccgctcggctctcttgaagctggcctgtgacctgaaccatgctccttgcatccagaaagctgctgaactcttctcccagtggatggaatccagtggaaaattaaagtagatgtagacttctgtcctaccctttgttcttttctctttgatgtaaragtctttgatcaagcaagacattaggtctaaaaccttttagtgaggatagaaaaaaaaacatgctgggcattacaaaccctgtttcatgctctcacattgtaagtgctatgtatggagactagtacaatctctaccatgaaatgtaatggtgtccattattccggtgctggctggatgcagtggcccacgcctataatcc

SEQ ID NO: 139 - DNA, 401 bp, Homo sapiens
cccacgcctataatcccagcactttgggaggccaaggagggtgaatcactgaggtcaggagttcaagaccagcctggccaacatagtgaagccctgtctgcactaaaaatgcaaaaattagccaagtgtggtggtgcacgcttgtaatcccagctacttcggaggctgaggtgggagaattgcttgaacctgggaagcagmagttgccgtgagccaagatcacttcactgcactgcagtctgggcaacagagaaaggccctgtctcaaaaaaaaaaaaaaaacttttcctgtgccaaattattataagatggtatcataacttctctcgctataactaaatctgtgagctttttgaaatcctttcttgaattcttctttttaaaaaagtaattcaagtttt

SEQ ID NO: 140 - DNA, 401 bp, Homo sapiens
ctataatcccagcactttgggaggccaaggagggtgaatcactgaggtcaggagttcaagaccagcctggccaacatagtgaagccctgtctgcactaaaaatgcaaaaattagccaagtgtggtggtgcacgcttgtaatcccagctacttcggaggctgaggtgggagaattgcttgaacctgggaagcagaagttgcmgtgagccaagatcacttcactgcactgcagtctgggcaacagagaaaggccctgtctcaaaaaaaaaaaaaaaacttttcctgtgccaaattattataagatggtatcataacttctctcgctataactaaatctgtgagctttttgaaatcctttcttgaattcttctttttaaaaaagtaattcaagttttcttcttt

SEQ ID NO: 141 - DNA, 401 bp, Homo sapiens
atcagccccctcccaccatcccttcttggctctctcctcagcccagaagaatcccaagatgccttgtatagtgcatcacacatatgtggcctcatttgttaatgcagttttgtactgggggaaaatctcaggaattttggtaagagctactcctcacccctagagcccctgtggaggtggcacagtggagaccttggttcmggtgaaagaaacctagtcaggagtgtttggggagccctccctccaaattgttttggttctgagtcttttctggggtttcaactttggggagaactggagctcatttcaatatgttcctggatctaaaatatccctcagaaataaccaataatgattagattttgttggaatggaatgtataagacagcattgctttctgt

SEQ ID NO: 142 - DNA, 401 bp, Homo sapiens
ctcacacagctttgcgtaagcaaaaagcacatttccactcctctcccaaatgctcaaggagttgacgtccacatgagaccaaatagaaactgctttaatatgtatgtttgtgtatgtttcctttaaaactctacttgagccattgatttgtctgttttcatgattgtcattttcctcctgaaatgatcagccttaatctamaatgctgtggatttcattttaaacaggaaatgatgcccctgcccaaccatgtgcagagcctagacttggaaccatatttttttctgagctgtaactgtgctctgctttatcaacatgaatcactttgacctgtagtacaagttttccatatataaaacattgggttgaaggatgcattctgtggccgtgaatgagagttc

SEQ ID NO: 143 - DNA, 401 bp, Homo sapiens
tgattttctggcgcagtgcgggtgtctcggcgtccgggatcgggcgggtcgcagtagggctccacatttgttgagtgactgaacaccgttcccggccggggagagcgccgcagccgggtccacttcaggtaggggctgggctttcccggccccgcctaggccccgcccccagcgcgaacccgctcccacctcgcctgtccrcggagcagcagggggtttgactgtgcttttccctcttgcttccctcgctctttctgcagctgccacgaaaacccggaacggcggagcggcgccgcccctcgcggcacctccctggcagcccttggaggccgcgctgggcatgctcagtcagctgggccgcctcagctctcggagtaggaagctcgggcgctccggctgta

SEQ ID NO: 144 - DNA, 401 bp, Homo sapiens
cagtaaggaagtaagtagaggcaggtggtagggtggcagtaagaattgattcccccaaattaactatgctgtttgtcctaattttatatgtgttgtagctttacccttcaaaaagaaagaaacttagttctatttacaaaggtagtaaattcagtttgatttaattgtgctttcaaaagtagtgtaaagggaaaagaaccraaccttaaaaaaattctgtaagaatattataaactcaaaatttatttccatggcttttgacatattgaaaataaactggggataaatacctaccttgaccagcaacctttacaccagtagccataaaatgaggccattcagataatgttattgaaagaggtgaagttcaatgccattcgtagtaataataatatctggta

SEQ ID NO: 145 - DNA, 401 bp, Homo sapiens
taattagtaatgtttgaagttgtatcaaatcaagaaatgtttagagcacagaagaaagcagaataattatctaataaagttcaagtagagcttaggcattagcaaaaaaacgcagccaaataaagtgaaaggttattatttggaaagaacagtgatatactagttcagattccttgggctacaaaagacaaaactctgagyaaaactagtttaaatgatacagacatttatcatttcatataacaagtccactgataagataatcttcaatcacagctcaccagcctcatcaagagcccaacttctctctctccactcttcagtcctctatatgagcaattccccaaagcccagactaccatatggtcaaaagttggctgcagcaggggaggaagaaggaa

SEQ ID NO: 146 - DNA, 401 bp, Homo sapiens
aggaagggcatatagtaattaaaatatttatcatgtgccattctctgctcttgcctttttttcccctaataaaggagaaagaaaggggtattagagaagggaatgcttttgaacaggagtgataaagttcaatagcatgtatgatgctagccctctggagcaaactgtacaggaaattttgtaactttgtagtaaaaacgkgttttttagtttcagaactttagtttttttgtaaaacagtagttgattttcgtagctcattgacaaatggtttttaaaaatcactgttagattacttcatctgggcttctgccatttaatattgcagttgctcactcttttttgctactcaatagactgaaaattgaagtgttaatctgttgatgactatagtaaattaa

SEQ ID NO: 147 - DNA, 401 bp, Homo sapiens
cattggttttgagggacatctctgatggctggagccaccttatctgtactgcttgcctccccaaccacctcatgcattatagaatcccaaagccaaggatgacagtgacctcatgtaaacatattctctgaatagaatattaccaatttagattgatgataggcttaaaaacactgacgtgttccttctcatctccctggrcatttccattttgtctcgttttttatgaagtgcccttcttacgtcatcctagccattgctgctgtgattcctataaaatggttaattttaaaaatgtactcactgtaaattcacaatagaccttgtcataacaagtttgaaaaacaaattcccattaaaccaacaaataaaattaaaaaaagaaatcagatactctctat

SEQ ID NO: 148 - DNA, 401 bp, Homo sapiens
tccttctcatctccctgggcatttccattttgtctcgttttttatgaagtgcccttcttacgtcatcctagccattgctgctgtgattcctataaaatggttaattttaaaaatgtactcactgtaaattcacaatagaccttgtcataacaagtttgaaaaacaaattcccattaaaccaacaaataaaattaaaaaaaraaatcagatactctctatgctatactctctttcctggcaaatataactaattaataaataaaattagtactattcatcttttctaacccaagatattatacttttgctgagttagtagcaatttacaggagacttaggaataaaaaaaaatgagctattgtttatctttctcttgaaatgcctctttaatcttgggcctc

SEQ ID NO: 149 - DNA, 401 bp, Homo sapiens
gattggatgatattaatgaataactattagttttcttcagtgtgataaggtattatggttctgtaagagaatgttctgatatctgtgagttgcatgccaaagtatttataaatgaaatatcgtatctgtatatcacttttatattgttcagctaaaacaagaaaaacatgtgtgcgtatgtgtgttatacacacttgaaartgaagcaagtgtcagagtgttaaaaaactgttgaatctagatgaacagtgtatgggtgttgtactatctttttttgtaggtttgaaagttctttaaataaaaacttaggagaaaaaataagctataaacaactattcttccctgcaggtctcattttcttgctaatttggttaatttgttataacatcaaactagttaat

SEQ ID NO: 150 - DNA, 401 bp, Homo sapiens
aaatgaagcaagtgtcagagtgttaaaaaactgttgaatctagatgaacagtgtatgggtgttgtactatctttttttgtaggtttgaaagttctttaaataaaaacttaggagaaaaaataagctataaacaactattcttccctgcaggtctcattttcttgctaatttggttaatttgttataacatcaaactagttrataatgttaaatatacctttaatattggattgagaaacatttaaacattaactatcaataaagaagtttatttttctttcattgctttataggtttatagattgtaaagtcacaaggtcaggaagtcctgacatccttccagcagtggttataagtgatactttttggcaggaaaataacttctagctgagtatatgcag

SEQ ID NO: 151 - DNA, 401 bp, Homo sapiens
gtttgaaagttctttaaataaaaacttaggagaaaaaataagctataaacaactattcttccctgcaggtctcattttcttgctaatttggttaatttgttataacatcaaactagttaataatgttaaatatacctttaatattggattgagaaacatttaaacattaactatcaataaagaagtttatttttctttcaktgctttataggtttatagattgtaaagtcacaaggtcaggaagtcctgacatccttccagcagtggttataagtgatactttttggcaggaaaataacttctagctgagtatatgcagcataaaggttccctactgcacagaagtcattaatttttttctgagttaacatcactaaaagtcccccttagctatagcagcc

SEQ ID NO: 152 - DNA, 401 bp, Homo sapiens
ggtttataccatacccagtgttttgtgaccatctttttagtgaatgatcagtctatgagattttccatgtcacttcatgaagcctgctttatatataaaaaaaaactaaagtgttttatgggttcataatattctgtagtatggccctatcataatttatttaatccatcacgttttgttgggcttttgtttttttctctyctaaaaatactgccacagtcttggagtggggcgtggtttctcactataaacagatagtatagcatagtagatgagaactgcttttgttcagatgtcgggctttggtatttattactgtgtgactgtgggtcaattagataacctcagtttcttcaactataaaattgagttaggtagtattaataaatacctactttata

SEQ ID NO: 153 - DNA, 401 bp, Homo sapiens
cggaaattttcacatttgttaacataattccatagcatgaatcatttaacagtagcattccatctaagttctaattaagcctagccttgcttggcaccaggttacttagctcagcgtggctacagactatttcatagcaaccatttagctatgcatattgaaaaatacctctgtatggccgggcgcggtggctcacacctktaatcccagcactttgggaggccgagatgggcggatcacgaggtcaggagatcgagaccatcctggctaacacggtgaaaccccatttccactaaaaataccaaaaattagccgggcatggtggcgggtgcctgtagtcccagctacttgggaggctgaggcaagagaatggtgtgaatctgggaggcagaacttgcagt

SEQ ID NO: 154 - DNA, 401 bp, Homo sapiens
atcgagaccatcctggctaacacggtgaaaccccatttccactaaaaataccaaaaattagccgggcatggtggcgggtgcctgtagtcccagctacttgggaggctgaggcaagagaatggtgtgaatctgggaggcagaacttgcagtgagccgagattgtgccactgcactccagcctaggcaacagggcgagactcygtctcaaaaaaaaaaaacaaaaaggaaaatacctctgcattgccaaggcatcagttaagaactcacattcagctagatgcagatgtaggttttttgcttctttctctcttttaaatcaataatggcatttctgggtgtacagtgtgatcttcgacaatgttaagcggattaattgtctgcatgttctgaactcttccctt

SEQ ID NO: 155 - DNA, 401 bp, Homo sapiens
aatgttaagcggattaattgtctgcatgttctgaactcttccctttttctgcccacctttccttcccaccgacaataagtatgcatagggctaccccggtttcctcagttgcagttccgggaggaggattccactctggctttggcattaaagacttttccttgcactcaggaacaacgctttaccagcagctgcctaatytttttgctcttgtttttgtgtttcttcaggttccctctggggtcctataccatacaaaatattgttgctggatcaacttacctgttttcaacaaagacacatttatctgaggttggttttataaaatgataatacagagactgggcaaccctccgcacacccggaccaggctgctcagttttagtgagatggtggatttt

SEQ ID NO: 156 - DNA, 401 bp, Homo sapiens
gttaaaactttgagtggatgcatagggcggatagctaacagtcacgggagctccatcaggaccattatttactttttggactaaagcagttcttgtaaacactcaggtcacctaagtagccaactgatgcagtagtcatacagtacctaaatcagtgtgagaaatgtcatacgtgtcgtatgccagtgaaaccaaggaacrctgtcttactttgaaggtgagacattggatgttatcagggaaataccccttgcgttgattcacatataagtaggagtatgagtgcacctttttagaggcacactgccacggttacattcctggtcaggtctacaaaaggagtttcttggttctgtctgcatgagtagccttgaggaagaactgagaatttcttaggcttc

SEQ ID NO: 157 - DNA, 401 bp, Homo sapiens
gagatacacagtttgaccacttggtggtgcccagaatgtgtaaaaaggtctaatacatgttctaggggagctcatctgctgagctcttaaagaattatttctgtttaagagttacattttatttaaaaccgacctcaggtaagggaaatgtatattttcttagtagaaattctagactatatataagaaatcagcgtaagraccagtttttgttctttctctttttaaatttcaagtttcatttaaaaaaatatatgctaacctttttagaatattcatcttggatactcagagttggcatttgtttactttggtaatagattcttaattttcccatatgctgttgtttaggaaatgctaattttaatgtggccaggttataattgtatctttaaatttaa

SEQ ID NO: 158 - DNA, 401 bp, Homo sapiens
gtgataacggtgtcattcacatttatgttgtggggagggatgaatgttatggctgtcagacaagatagagaagaaaatacacaaaatgtgtgagatacagtctatgtcatcaagtagctgaaagttcagatggttggtacttgtggagcaataagagaggtaatatgtgctgagtgggacagaatttttgcctaggataakgaagagaaacttttgggaggtaaatgggaattgagttggcttaaataattaaaatataggtaaagaggaatgtgaagtatagggtagggcaggaatggaacaatgaggtctgcaaagaaaatgaagtactaatgggcaagtgatgttttttcagagagtcagtgtttgaccacagtggaagccacacgtgaagagggaaa

SEQ ID NO: 159 - DNA, 401 bp, Homo sapiens
gtgcctactatgtagtgggcattgtttttgtcattggagttgagttttcactttttcctctcagcttactgcatagatgaggaaataaatgtgtaacagatacaggatgccatataaattgtattgaagatggagaaatgtgatttctatttcaaaccgggaaggtcttcacagaggaggtggtgcttatttcaggattgsgaatgatgacaagcacatcaatggatatagtgtggccttaatattatgctccctgccctatacatttgttagttccaagcatttgaagtgactctgcaggaagaagagagattctggttacttcacaacacaatatactaaaataagaaattaatgacctacctagcagggagacttttgagagccttgtcaatttattg

SEQ ID NO: 160 - DNA, 401 bp, Homo sapiens
actttctagctgttagtcttttgcccagaagatataccttccctactctctgaattctctttgagaacttattacctaacagttgttcccaaatcattgtttttttcctctgatcatatagtagtatttcaattgaaagccacattgtttactttatacttactgtcttaatctgttggcatttagaacatatttctgcckttctcattgactgcaacttttgcatcatggtgtttttccatcccaaactagttcctaacattatcctcaggtttttcagcacccacatcaaattatgcattggcctcttcacttcaatactgcttcgacacccaaaagcaccttggttttaggcaagcttattttcttctaatcctctggttcagaaataaataggtcag

SEQ ID NO: 161 - DNA, 401 bp, Homo sapiens
tgcaccagtgcactcccgcctgacaacagagtgaaactccatctcaaaaaaaaataggtcatatagataagatgtcttttgggggatctcctctaggtctgttatttctgaagccacccatcaccatttgggagtatttctctgttatttcctctttaggacttagaaatcatcttcctctgaaacaggttttaaaataayatcttagaaaaatgttattgtaattctcaaaggtgttttgttttatgcagtaacctcgtcctttcgctgctgatttgagataagcccaagaccacactgaccaaattacatattttacaactacttttcatttcaaggatttttttagatacattttttaaggagaatctcctattatttttttcctttttcttttcttt

SEQ ID NO: 162 - DNA, 401 bp, Homo sapiens
gtattctattattttctttctttttcctcacttttttttttaatagggatcttctctcttgttgatgttgaaaacttaccttagtgaagatgtgtttcaacatgctgttgtcctttacctgcataatcacagctatgcatctattcaaagtgatgatctgtgggatagttttaatgaggtaagtgacctgggtaatttatktagctcttactgtaaaaagagaggagttcgtctatttatactttttagcatgtgtgtaagttaatctgtggtacaaagcatagttatttaagaaagggggggatggagcttgctatataaatatttatgaatggagcactaaattttatgtcaagaaatgggagtgctgttcttagttgttggaaaagacgtgtgtgggc

SEQ ID NO: 163 - DNA, 401 bp, Homo sapiens
attctataagagaaaagacaccaattttaaaacttgagaaagtactttaattctgtaggcaaaggttcagcaaatcagctagcactaatcttgaccaaatgggtgagtcagcctcatcacagagattttttttttaatttagatgaaatttcacatttaaaaacatggtaactccaagcattcttccaaaaacaaagaatraacattggaatagtcacttacaaggacttaacgacttgtattaaacatattttacactaaagtactagatggtctctagttcattttagctcaaaccttccttaattcttcgagtaacttcaccagactattctttaagtcttagtaattacaaatagaacctgaatgagaaagaaaattcagaattaaagttgttatca

SEQ ID NO: 164 - DNA, 401 bp, Homo sapiens
taaagtattagccagcctctgacttaagcaaatagagaaaaatcactgtttttaaactatgctagtaatacactaagaatgcccatgataaagaatttaaacagtacctaagaatatagagtaaaatatgaaagtatttctcatgatccttcacttccattctcgttttctctgttaacaacagtgtcagtcctggtcttygtattcatctgtgggaatggatatttgtacatatgtacgtacatacatacacacatacctacatatttaactgcattttaaaaaccatattaggtttataccatacccagtgttttgtgaccatctttttagtgaatgatcagtctatgagattttccatgtcacttcatgaagcctgctttatatataaaaaaaaacta

SEQ ID NO: 165 - DNA, 401 bp, Homo sapiens
ttgtacttagtcaaaatcattatgtatatatgatttctgtatcttgccttttttaacttaacatatatagtaattgacttaacctacttgattcaccatttgttgttataaaatattgccttaattaaatatttcaaatcttaaaggtttttccattatatgaaattttttaaggaatgcttttcaaaaatgaagactgcrgtaccttttgtgatttggtacatataaccaaatcgccctttttattgtgtagttacagttgataatgccacctgaaatacatgaatgtgccagtttcattgcagttttaccatagtggagtgttaaagcagcaacaatctaattatttaaaatcctagtggtagttggtaatgtcatcatatctttgatagcatagctgg

SEQ ID NO: 166 - DNA, 401 bp, Homo sapiens
ttttaagttagaaaaatgtatctgtgtgggagtaaaaagatttcctttttaaaatcatttcagatatcaccatacttgattgggaactccatgtagataccttgaatattaaagtacttctttctactgctttcaagatagcagcacagccatcactaagttaaagcttattttaaaagctggggttacctgaggtttatygtccttttcttcttttgaaattcttctgtgtgaaacttacaggttcaggcattctttgaaaatcagtcagaggcaaccttccggcttcgttgtgtccaggaggctttggaagtcattcagttgaatatccagtggatggagaagaacctcaaaagtctcacatggtggctgtagcatgcacaaccgcacctcattttgtt

SEQ ID NO: 167 - DNA, 401 bp, Homo sapiens
taccgtgcattatggagagaagattcaagcatcagatgaagagttggggtggaggatttggataacgttttgaggtctttcccagctctgagatcttaataaaggcagtagactgtgttttctccctgcaccccatttactgctatagttctaccatgaaacttatcacactgaattgtaatgcatatagttattgctctrtacttcgtagtagcctgtgagttctcagaggacagaggctctcattccttttccgctccctagtgtccagccagtcgcctgcctggttctttgtgagtactctgtgaatattaaattgaactgatgtatccatagacacactactaggaagatagcagtcactgaattaaactttttctcaaccctaaattgtgtactca

SEQ ID NO: 168 - DNA, 401 bp, Homo sapiens
tcaatgagtattgccattgttcttcactgattttttttttaaataagatttcaagcatgtgattttttttctcacattcttcatttgttcctatttgacagttatgagtaggatttgaatttcttttgttctccagtcatttggaatggttttctatcataatgctattgagaaggtaaggccagtgaagacaccacatarcaatgcagttaggtatgtcaagtgggatcccctgcattgcttgtccgttccttgcatcgtcagcgtagcaagtatttttctacttcatgtcctcccagtgactcaaaagctttaccacttacacattccacaaggtgtctgttctgtgtttcattgcttttgaaacaaacacaagcgagttgacatgttatacaaacctt

SEQ ID NO: 169 - DNA, 401 bp, Homo sapiens
aaaagagaggagttcgtctatttatactttttagcatgtgtgtaagttaatctgtggtacaaagcatagttatttaagaaagggggggatggagcttgctatataaatatttatgaatggagcactaaattttatgtcaagaaatgggagtgctgttcttagttgttggaaaagacgtgtgtgggcttgggtagccagttktttttttttttcctgtaccttaacttctattcctattttgtaggaaagttgtcttctccgtattaatgaatattactatattttcattatttgactttttttccagaaatctcttttcctatccttacccttttagtttttctgcctcttttgaatgattctgtactctgctctatgaatctcttgcctttgtgactgtt

SEQ ID NO: 170 - DNA, 401 bp, Homo sapiens
gtctcgatctcctgacctcgtgatccacccactttggcctcccgaagtgctgggattacaggcgtgagccaccagcctatcttttttttttttttaaagcattatagtctttgcaccttcttttcacaataaatcttgaatttatttaccctttaggcaaattcagaattcctgaacttaaatcccagctcaccatttacyctatgacttgggtaaatcatttaacttctttagccacattgtggtcacttgtaagatgaggatttataatttttgtcttactttacctattgtttgaaaataaagtgaacaattatgcagaaaagtagaaaataaccttttagaggttggcagagaaatgcctatacctgtgtgtatgtaatttgcaagctcttttgaaa

SEQ ID NO: 171 - DNA, 401 bp, Homo sapiens
tcaaaataaatgttcaaattgtggaattgaaagtaaacttagatataatctgatttgaagttctcattttagtttttaaaaaagttgtttgtccaatgccatatagcaagtaaatggaagggacaggattaaaacctgacacttggccaggcacagtggctcacacctgtaatcccaccactttgggaggccaaggcaggyagatcatggggtcaggagttcgagaccagcctggccaatatggtgaaaccctgtctctaataaaaatacaaaaattagcagaatgtggtggcacgcacctgtagtcccagctacttgggaggctgaggcagaagaatcacttggacccaggaggcggaggttgcagtgagctgagatcacaccactgcactccagcctgg

SEQ ID NO: 172 - DNA, 401 bp, Homo sapiens
attggattttgttacacgttcatcctcttttaatggaatctttcccacttacactttttcatgtatccctatatatgtagagaggtgtatgagcttaacaaaaaacagtttcagtaatttaggaccacatatcttttagttaaaatcttgtcagtggttccatctactgacctatgcatttgtaaaggaagtgaatttagyttatatcttgtcactctagccttcaatactcatctattccagtacgttttttttgtagtttccctgttttctgtccaaagttgccactggtatgacctatttttgttgggccctgctctctacctgttgataattggttcatttgatgaatatcctatgttaacctgttcaggtaacatacttctgcaacccatttaaaa

SEQ ID NO: 173 - DNA, 401 bp, Homo sapiens
aagagaaatgaatagaagaacctagttttgttgtcatgctaatatgaaatatggaaacacagaagaaataaaaaagcaataaagttttgtctaagacagtattctaattatgaaataaatgtacagaaactgttcataactgtttgcatgtctactaatttagtgtaatactcctattaggaaacagcagtattaaccctstctatgaaaacattaggaaattgagatttgaaagagtttagccaagattaccccagatgttgggatggacctagatgaggcctgtggttcttggcagtcgaggtgaggtcagtgcagctatgttttgtcaattagtcacctcatgacttgaaaactgtagggcagcagtccccaaacttttcgggaccagggaccacagt

SEQ ID NO: 174 - DNA, 401 bp, Homo sapiens
cctggtcccgaaaagtttggggactgctgccctacagttttcaagtcatgaggtgactaattgacaaaacatagctgcactgacctcacctcgactgccaagaaccacaggcctcatctaggtccatcccaacatctggggtaatcttggctaaactctttcaaatctcaatttcctaatgttttcatagacagggttaayactgctgtttcctaataggagtattacactaaattagtagacatgcaaacagttatgaacagtttctgtacatttatttcataattagaatactgtcttagacaaaactttattgcttttttatttcttctgtgtttccatatttcatattagcatgacaacaaaactaggttcttctattcatttctcttatttaggta

SEQ ID NO: 175 - DNA, 401 bp, Homo sapiens
aggagaatggcgtgaacctgggaggcggagcttgcagtgagccgagatcgcaagccactgcactccagcctgggcaacagagcaagactccgcctcaaaaaaaaaaaaaaaaaaaaaaagataactagaattaccaacaatagttttgttaaaaagatcattaagtacgcttccaaactttaatataatcactcttgcatygtaatacaatatgaaagaaataatacaaaagggctcacctctcaagtctattttcattttgaatgctatgaatacacgtattttaagtattttaagagtcaggggcttttttttgctgttgttttttgtttttgtttttgttttttgtttttttgagatggagtctcactctgtcacccaggctggagtgcagtggtgtg

SEQ ID NO: 176 - DNA, 401 bp, Homo sapiens
ctggagtccagtggtgtgatctcagctcattgcaactccgcctcctggattcaagtgattctcctgcctcaacctcccgagtagctgggattacaggtgatccaccagacctggctaattttttttttttttttttttgtattttagtagagatgggttttcaccatgttggccagactgacctcaggcaatttgcccacytcggtctcccaaagtgatgggattacaagcatgagccaccgcaccaggcctataagtatttttgtaagtaaaaactatgtatttgaatatgtctcaggattttcaagaaatgcaagtaaaaaataggagctgtgaaataatttttgattgttggattttgtttctttaaccacaaaatcacacatcagttggaccataag

SEQ ID NO: 177 - DNA, 401 bp, Homo sapiens
ggagtccagtggtgtgatctcagctcattgcaactccgcctcctggattcaagtgattctcctgcctcaacctcccgagtagctgggattacaggtgatccaccagacctggctaattttttttttttttttttttgtattttagtagagatgggttttcaccatgttggccagactgacctcaggcaatttgcccacctyggtctcccaaagtgatgggattacaagcatgagccaccgcaccaggcctataagtatttttgtaagtaaaaactatgtatttgaatatgtctcaggattttcaagaaatgcaagtaaaaaataggagctgtgaaataatttttgattgttggattttgtttctttaaccacaaaatcacacatcagttggaccataagtg

SEQ ID NO: 178 - DNA, 401 bp, Homo sapiens
ccactgcattccagcctgggcaactgagcaagactccatctcaaaaacaaaaaaaagaatacctaaaaacattttttatatcagaatttttattctttctagtggtattcataaaagcatattgcatatgatgctttttaaaatatcatgtgccctcaccccccacccgccatgcacaacttgcagaatggaaatacttcracatggtattaacaggtttggtgtttttattttggagagagatgaaaaaggcgtctgttagtaccttaataccgcaagtatacgtttagcaatgacagccaataccaatggactagattggatgatattaatgaataactattagttttcttcagtgtgataaggtattatggttctgtaagagaatgttctgatatctg

SEQ ID NO: 179 - DNA, 401 bp, Homo sapiens
  misc_feature (201)..(201): n= absent or C
tactcattaattctttttcaaatcctttaaaataattttaagacagttgaacacagtccacatctatatgagactaagtagcagtatattataactaagttctacatatgaaagtaaatttttagaatgactgtagtttgaattttagattcccaattcgataatctatagtattctattattttctttctttttcctcanttttttttttaatagggatcttctctcttgttgatgttgaaaacttaccttagtgaagatgtgtttcaacatgctgttgtcctttacctgcataatcacagctatgcatctattcaaagtgatgatctgtgggatagttttaatgaggtaagtgacctgggtaatttatttagctcttactgtaaaaagagaggagttcg

SEQ ID NO: 180 - DNA, 406 bp, Homo sapiens
  misc_feature (201)..(201): n= absent or T
ttggccagcaattaccagatcattttaggccagcagtgtaaattcctgtgttattttttgtcacatcatgcttataatcatctcaaaagataaagtaatcatcattactctgtgtttataagtgagaaaactgatactaagggacagatttgcccaaagtcaccaagtcagtgagaaaatcagtacttaaaatttgtcttnctgactaagtccaatagttattcaattatatcacagctagttcctagttttaagaaaagtcccccatcaatcttcccctaaaggtcctagattttgaccaactctcttctgacaccaaagggccctgtagtattaaaataataaattactgaaaatatcttgcccaccattgtgtcacataaagtcaattctaatacatgtcaat

SEQ ID NO: 181 - DNA, 401 bp, Homo sapiens
  misc_feature (201)..(201): n= absent or T
caaaaaccaaattttaaaaattagtagttttatcacctaggcagaaaacttttctattagaaattatacagtctctcatctcaaataccatgttttactgttctaagaatcaaatagtgctatagtgaaaaaaagagagtagtttttttctgaaactatctactatactgtataacatgagaaatttctcaaaaaatatgntttttttttccataatcatgtctggtctcttttttgagaccaagatgaaaccatgttgcttggtcttcaaccatcagcttatctgttctctaatgatttttgttgttctggtttggtcttaaagttttgagaattcattttattataaatgtgaaagttcatttaaatattgtgttctttttgttcctgtaaaggaaaaa

SEQ ID NO: 182 - DNA, 401 bp, Homo sapiens
aggttgcagtgagccaagattgtgccattgcactccagcctgggcaacaggagtgaaactccatctcaaaaacaaaacaaaacaaaacaaaacagaaaacccaaattggtgcttcaagaatatgatgttatttctcaaaggtacaatctagctgaaatcatatacaagtaagtaggtgtggacttttactgttgagctaargtttatgtttatatatgttttattctttaagctaaacaaacattcagataacattctatgcattttttgaagcatagggttagtaatgaggacttagattttttaattaaacaactcagtaactatataaaaagaaaaggagtcccttatgaataaatattaaaattaaaagaaataggcaactataaaagtaagtattt

SEQ ID NO: 183 - DNA, 401 bp, Homo sapiens
  misc_feature (201)..(201): n= absent or A
ggcaaaaagaaacattccacttgaatctaacactctttacaaagatttcccacccaatgacttcagctagaccagaatgagtcatagcctcaccaagtcacaaggtagcctgtaagaagtaactctcttatctggacttgttgccttcctgaataaaatcaggattccactggaaccaaggaagggaaatgggtatcaggnagtgactagctgtgtctactacatcctgctcttcccttccccacttgggtgctcactgcacagcctgcagccatccacctaggacaactcttccccaggctcctctctttccacattcccttggtgacacttcccctcattgcagccacaatcctcaggggcttgttttcaggctcagcacagtattggataggaaaagt

SEQ ID NO: 184 - DNA, 402 bp, Homo sapiens
  misc_feature (201)..(201): n= absent or T
ttgatgggattgttttattttcttgctgatttgtttgagttccttttagattctagatattagcctttgtcagatgtatagattatgaagattttctcccactttgtgggttgtctgttcactctgctgattgttgaataagatgtcctttccccactttatgtttttgctttgagaaattgttgacagttttaaaatcantaatgagaaactaaaattggagttaagagttcaccaatgtgctttttccaaattatgaattgttcaaaaagtttccattttccacctgttgagatcttcattttgaggtttttattttctactgtgtctaatctacatcccacttttccaggtgagtatagagggctttttaaaatcaattagaaaaaaataaatactgtt

SEQ ID NO: 185 - DNA, 403 bp, Homo sapiens
  misc_feature (201)..(201): n= absent or A
aactaaacatttttctccctgtttaggatgactattttttgaatgtgtgttttgaagtaattacaaaagattcattgaattcatcccgccctatctccaaaccagcggaaaccccgactcaaatacaggaaatgtttgatgaagtttcctataacaaggtagtaaatatcaggtgcaggtggaagctctgctttcagaagnaagtaataatgactatagaatcagcagtttaagtcactggtgccagttcctgctacttgtttcactttttatttgttcacttttcagacatcacacatgttccgtaaaatttaagcttcctttaaaaacataccatactaccattttctttatctcttttttcaactcttttgtttttttttaaagggagcttgtattttga

SEQ ID NO: 186 - DNA, 401 bp, Homo sapiens
  misc_feature (201)..(201): n= absent or A
ctcttatcagaacgtaaaatgtgccagactcttagttaaatctctcctggatcaaaaaaagacctggggtggtgcagtggctcacacctgtaatcctagcactttgggaggccaaggcaggaagattgcttgaggccagcagttcaagaccagcctgggcaacatagtgagagcctgtctctacaaaaaaattaaaaattnaaaaaaaaaattagtcaggtgtgatggtatgcacctgtggtcccagctgcttgagaggctgaggtgaaaggatcacttgagcctgggcaaagtggaagtgagctgtggtcatgccactgcactgcagcctgggcaagagagtgagaccctatctcaaaaaaaaaaaaaaaaaaagagatcagaaaggtctttttctatag

SEQ ID NO: 187 - DNA, 402 bp, Homo sapiens
  misc_feature (202)..(202): n= absent or G
cagatgccttggttatgtgcggattctaccgtcatttatttcagccctagatggtgctaaagtagagagagacagatttttcttaaactattgcctttaaaaatcatttatttttatccccattttttttgtttatatccaaagggttttcaacaagctgcccctttcccaacaccccagcccctcaacgaaaacataatangagacacatcatttaatttctcagccctttcatgatctcttagactaatcttagttttcataaattaaaggcctacttggctaagttcatttactttttttttctcctacttttcttgatctctggacccaggaatcccagatgatacaaaaccctttgtttcatacctgccctgccatagaatgatctagacctttaag

SEQ ID NO: 188 - DNA, 401 bp, Homo sapiens
  misc_feature (201)..(201): n= absent or A
gaagtaagtagaggcaggtggtagggtggcagtaagaattgattcccccaaattaactatgctgtttgtcctaattttatatgtgttgtagctttacccttcaaaaagaaagaaacttagttctatttacaaaggtagtaaattcagtttgatttaattgtgctttcaaaagtagtgtaaagggaaaagaaccgaaccttnaaaaaattctgtaagaatattataaactcaaaatttatttccatggcttttgacatattgaaaataaactggggataaatacctaccttgaccagcaacctttacaccagtagccataaaatgaggccattcagataatgttattgaaagaggtgaagttcaatgccattcgtagtaataataatatctggtatccaaag

SEQ ID NO: 189 - DNA, 402 bp, Homo sapiens
  misc_feature (202)..(202): n= absent or T
gtaggttactgatttgcccaaagtcatgtggttagtaaattatagagtctgggtcttctgactccaaatctcatactctttcttttctccttatcttctagtagtggaaactaagcccaaaatgagagaggctaccacctccaagtggtggttgtatatgtgctatattgattggtacctgaaatatgcacaccagggccantatatttgccgtgattatagccacgctgggatgatctcccaagttcagatctagttattcttttacttaactgaaaatctgcatttctccttgtttctttttatgcttttccaccaacctgtaatcgaggacttttctttttttttcccttgagacagcatcttgctctgtcgcccaggttggagtgcagtggtgcaatc

SEQ ID NO: 190 - DNA, 401 bp, Homo sapiens
actacctcatagaagaaaatatttaaagctctttctgacttcatttgtttatatatgccatctttttttttttgtttttaaagaaacaagatctcactctgtcacccaggctggaatgcagtggcatgatcatagctcactgcaattttgaactcttaggctcaactgatcctcccgcctcatcctcccgagtagctaggmcaacaggcatacatcaccatgcctggcttaatttttttgtagagacagagtctctctatgttgcccatgctggcttgaactcctggccttaagcaatcctcctgccttgccctcctaaagcactgggattacaggtgtaagccacgatgcccagcctgtatatgtcaacttagtcttaaggaatgttgtttgaattctgt

SEQ ID NO: 191 - DNA, 403 bp, Homo sapiens
  misc_feature (203)..(203): n= absent or T
gtctcactataaaaatttctaggaaaagaacatgcaaagcctgataaaatgatgttttctttttctcttcctcttcttagtaaagaggaatatacaaaattcactagaatataattgatttaatctagagctggaactgggccaatacatgatgaaagtagtgtctgttacttcctcttctcaactgtgttatttcccttgcnctgctgctgctgctattttaattcctgccatttcgggtttagagagtccacatgaaaacttctgtccttacgtttgaccctgaggacagctgagccttcttggttcctaatgctccagtgagaattactcttaatttaactgcatttttattttttctattctcaaaagaaaggtagcagagagggtgacttcaggcttc

SEQ ID NO: 192 - DNA, 405 bp, Homo sapiens
  misc_feature (201)..(201): n= absent or T
aaatgaggtttcaccatgttggccaggtgaactcctgacctcaagtgatccgctcaccttagcctcccaaagtactgggattacaggcatgagccaccgcgcccagctgaaagtatatatactttctagtttgtgatatattctaaagtatatctaaaggttggtattttggcattttgaggtcccagaactgaaaacatnaattaatagttttttttttcatatgaatgtaggtcctatgaatcacttttattaccgcagtgtggtgcataatcaaacaaggaggacttagttgtcttaaaaaattatttttgctttcatgttcagattggtatccagttgaaagtatttttcaacttcaaaaatgggaagtctgggcaacaaatgagatatttgtgggttgta

SEQ ID NO: 193 - DNA, 401 bp, Homo sapiens
  misc_feature (201)..(201): n= absent or A
aaccctttgtttcatacctgccctgccatagaatgatctagacctttaagaggactagaatcagccctctttttctgggctttctggggccaggaatgactaggattgatctgctttctcaagctttgccccgggcctaaccaggtcagcctgggaccagcccgtggggtttgactatacctggaacagatggttaatctnttggcttgctataatgtaatttccatttggctggcagtagggaaaggaaggtacttcctgtaagctacacactgattttcatccaggtgttcacacataccgggttttatgaaagagagcttgaccctcgcattcctgattagcattttgttagtgtgaaagtaaggtatagacacagagacaggtataatcacaaaatg

SEQ ID NO: 194 - DNA, 401 bp, Homo sapiens
cccaggatcatcaacccatcaaatttcacttgaagtatttcattatggccatgtaagcacaggttccaactgaaggaagagtgattttgccctagattggaatgccagagtaccaggggatataaggagaaatatttttagtagaaatctttatttgtaaggtttccaattctgtgcttcatgtgtctgtatagtcacttyccttcttttcccaaatgacatttgaaggctttgctttgaaaggttttagaggataaatttaatggctacttctcgtaataaaattccagtatgcacaccacagttcagagactgagtactgtgctacttgacgttgtgttaggtttagtagtctctaagttcccctctagaggtaaatgagatgatttattttgtttcag

SEQ ID NO: 195 - DNA, 401 bp, Homo sapiens
tcaaagccatttttttgtctttctctcttttaattttgcttagttctattagagaagcttttataaatttttcttctctgaggtatgattagaatacatatttatactggtagataaagtaattaagggatgtatttcttgtttttacacatagcttacatttcctggggataataggcattatagaaggagaactaaagrcaagaactttcaagttcccattgcaattatacatttgtgttcaatcccagatctcacgcaagaattgaaatgcagggccagtatgccatttattttaaaagtattacatagagggaaaataaaataaaaattatttatctgaatagaattatggatcttgcttggtctctttctccatttaagaaggatcaaaaagtttc

SEQ ID NO: 196 - DNA, 401 bp, Homo sapiens
cgtctgcctttttcttgttgatttgtaagagttctgtatatatcctagatatgaatcttttgttggtcatgtatatttgcaaatatcttctcccactccatcttgcttttttttactctcttaatgattttttattaatatgagttttaaattttaatgtaatctagcttatgaaatcttttcctacccctagatattctstgttctcttctgaaaactttatcattttatcctttacatttagatctgtgatccatctggaattgatttttgtggatggtgtgaggtagacaccaagattcattcttttcagtatggatatccagttatccccaggaccagtatattttattgcatagaattatgtttgaggtaattagtatgatatcaacaccatttgg

SEQ ID NO: 197 - DNA, 401 bp, Homo sapiens
  misc_feature (201)..(201): n= absent or Y
ataacagaaatttattctctcatagttctggaagccagaaggccaaaatcaaggtattggcagagtaaggtttgctccttctgaggaagaatctgttccattcccctctcctaacttcaagtgattgccagtaatccttggtattcctgggcatgtaggtgactaaccgtggcctttgtctctgtcaacacagtgttctcnctgtgtttctgtgcccaaattgccccattcttagattaaggcccaccataatccagtatgacctcatcttaacttgattgtacctgcaaagaccctattcctaaatgaggtcatattcataggtcccaggcagacacaaaatttgaggggatactattcaacctagtacaggtagcaataaataagattagtgcatatca

SEQ ID NO: 198 - DNA, 401 bp, Homo sapiens
tcatatggagactaactagtaaaattgctccctgtaattcggtggtgtaactgctcaggaattagccacagccatcttcaagtgtcagatttcctttgcttccaggacttcaagtgccattctttccattgctgcctttgtgttttagtaaactctcaatgagtattgccattgttcttcactgattttttttttaaatargatttcaagcatgtgattttttttctcacattcttcatttgttcctatttgacagttatgagtaggatttgaatttcttttgttctccagtcatttggaatggttttctatcataatgctattgagaaggtaaggccagtgaagacaccacataacaatgcagttaggtatgtcaagtgggatcccctgcattgcttgtc

SEQ ID NO: 199 - DNA, 401 bp, Homo sapiens
cctactttaatttgtggttggaaaattctataaggttgcctacattccctcattttgtgtctgctgcagacttctctaatgacttactactgactttgttcccactaagctttcttgggggtcctcaacatggcaccccatgaagccatttcagatcatttgaagggatgtcgcagctagagctccttctgtggatgtatytgtagcagtagagtggagcaatcccaggtcataaggaaggattttggttttggaggtgttctaatgggagaagcagaaccaatgtgactatctttaacttaacatttatttggtcatctttgggactaaaaactccttgaggagtttcactgtgctccatatgtcctcaggatgaaggatggtacaacagactgagacta

SEQ ID NO: 200 - DNA, 401 bp, Homo sapiens
tttggaggtgttctaatgggagaagcagaaccaatgtgactatctttaacttaacatttatttggtcatctttgggactaaaaactccttgaggagtttcactgtgctccatatgtcctcaggatgaaggatggtacaacagactgagactaggagccatgctctttgcagaaattcatactgagaggttataatatgctrgcatctttaccatttatttcctatttgaattttcagtttctcagtttgtttgttattgccatttattcctatagttacagaactgtcttttcccctttgcttgtagaagtcatcagtcgttctagatgatggacttgttcaggatgagttttctgagagtgtgaagatgagcacttacttggttgctttcattgtgggag

SEQ ID NO: 201 - DNA, 401 bp, Homo sapiens
ctgtcttttcccctttgcttgtagaagtcatcagtcgttctagatgatggacttgttcaggatgagttttctgagagtgtgaagatgagcacttacttggttgctttcattgtgggagagatgaagaacctgagtcaggacgtaaatggaaccctggtatgttgatgtggtaattgtctgaaagcctgtgtcacaagaggytcagaggacctcttgctttaacgattcctggtatttgctgtgtgaaataaataagcttttagatcacactctgacattttataccagaaatgctacttttttgcttagctgttatttactgttactagtttaatagctgaaagtcaataattttcaagttttaaaaaattttacttttaaagagaattttagtaagacac

SEQ ID NO: 202 - DNA, 401 bp, Homo sapiens
aaacaattattcttgatgatagaaatatgatagaatgtcttagtttctgttttcgtatttttgagctctaccagggaatatactgctagttttgggtttcctttctagactaaagagctttatattaatcacaggatacttggatcttatcattttgctacttcaaaagggtatatgtttcctattagaaaagactatcaygtctatcccatacctttcagagataaggacagagataaggattctctggtgatttatcagtaataatactgtatgcctttaatgatgctactcaaaaagtaaactaagtttttaatggtaagtgtagactgtaatattaagcgctaaataatggttcactcacctttggattgaaagactttatgtcaaggatttttcag

SEQ ID NO: 203 - DNA, 401 bp, Homo sapiens
ataaggacagagataaggattctctggtgatttatcagtaataatactgtatgcctttaatgatgctactcaaaaagtaaactaagtttttaatggtaagtgtagactgtaatattaagcgctaaataatggttcactcacctttggattgaaagactttatgtcaaggatttttcagaatcctttcaaaaggatattatractggcttaaatctgaaataattcaattaattccacttcaggtgttgcactacatatttagcctttgatttagagtttgcagccttgataaagcctaagaagcccaatctaaaagagtcaggtttgctgctgcttcaagactcagctgaatactacgttctccatgaagcatttctttctttccccagctggaattaatc

SEQ ID NO: 204 - DNA, 401 bp, Homo sapiens
tctgttcactctgctgattgttgaataagatgtcctttccccactttatgtttttgctttgagaaattgttgacagttttaaaatcataatgagaaactaaaattggagttaagagttcaccaatgtgctttttccaaattatgaattgttcaaaaagtttccattttccacctgttgagatcttcattttgaggtttttrttttctactgtgtctaatctacatcccacttttccaggtgagtatagagggctttttaaaatcaattagaaaaaaataaatactgttttgtaaaacccattgctttgaacatggctgtttaacacttgccttttgatacttcctgaataaaatgtttatagtttgtcgcatcatatatgtttaatttattcatttagcca

SEQ ID NO: 205 - DNA, 401 bp, Homo sapiens
ttatctagtatcccacttctcttaattacacagaatatttttgccaaattcccaattctgaaacaagtagttaccatgttgacaggggttgacaatgatatggaaactacatattcagaagacactgaattctgggtttagagggtttgggtcagtggcagacagaactctgttatgactctgttctgttattatataatraacttagagcattttaagcaggttttcaaatcctgaaataactgctctaagtttggtataataacagaacctcaagttttaaattttctttattgacaggtccactatggtcttcaaaaatcacaccagtagctctaattggaatagatttaatatagctaactgaactcctgattttcttgtttgttaatagtacctgt

SEQ ID NO: 206 - DNA, 401 bp, Homo sapiens
atacgagattgagataaaaggttgctttgcatgttcaagcaccagcaaggatccagtgttatctgaagtagagtaagcagaggagaaagtagtagatgaagttagagggactgtaagaggccagattattatagggtctgctaagccatagtaaggaccttgattttattctaagtgaaacgaaaagcaatttgatcaggraagtaccgtgatatggcttattttgtaaaagatcactctagtcttcaacagtacatggagtaaagaggactagttaggaagttattgtaatagatgggtggcgagataatggtagcctggacaagggtggaagttgtgaaggtgatggaggtacaactggacttggagtgtgttttaaagattgatccagtagaatttgt

SEQ ID NO: 207 - DNA, 401 bp, Homo sapiens
tcccagcactttgggaggccaaggtgggtggatcacctgaggtcagcagttcaagaacagtctgtccaacatggtgaaaccccgtctctactaaaaatacaaaaattagctgggcgtggtggtggtgcctgtaatcccagctactcaggaggctgaagcaggagaatcgcttgaacccaggaggcggaggttgtagtgagyggaggtcgcaccactgcactccagcctgggtgacaagagtgagacttcatctcaaaataaataaataaataaataaatacttacagtagagtgatgattagaagatggctcaagagaaaatggaaagagagcaaatggagatggcaaattgaggacaactcttttgaggagttttactacaatgggggaacaaaaaaaca

SEQ ID NO: 208 - DNA, 401 bp, Homo sapiens
actcgggaggttgaggcaggagaatcacttgaatccaggaggcggagttgcaatgagctgagatcacaccactggactccagcctggtgacagagtgagactctgtcttaaaacaaaacaaaacaaacaaacaaacaaaaaacatataaagatgctctttactatccatttccatcacccaccgtcagtggtccagacacwctttctccatgcttccgcttaagcttctcagcaccaagtattgtgttgcttctgtctctcatccctctccatttccctctcccttgccatgtgtgtgtgcatgtatgtatatttgtagacatcagtttagctcccctccaacacggaagaatctatcatttggtgtgcatactggcagtagagggtgggagttaaaaaga

SEQ ID NO: 209 - DNA, 401 bp, Homo sapiens
agttgcaatgagctgagatcacaccactggactccagcctggtgacagagtgagactctgtcttaaaacaaaacaaaacaaacaaacaaacaaaaaacatataaagatgctctttactatccatttccatcacccaccgtcagtggtccagacacactttctccatgcttccgcttaagcttctcagcaccaagtattgtrttgcttctgtctctcatccctctccatttccctctcccttgccatgtgtgtgtgcatgtatgtatatttgtagacatcagtttagctcccctccaacacggaagaatctatcatttggtgtgcatactggcagtagagggtgggagttaaaaagaaaatttggccagcaattaccagatcattttaggccagcagtgtaa

SEQ ID NO: 210 - DNA, 401 bp, Homo sapiens
gttgaccataccagttaatcttatttacagaggatgtggagagtaatgattaatatgttgagctgatgaagtagacaagtggctgctgtatgtagaagtaatgttggaacaaataatacgtcccagaatagtttctgtaaggctgattttactctgaaattttaattaatttatagttaatataactacctctgtattttkttgtagtcttttgtgggtagagttgaggaagagataggaatgggattattttgacatggctcatgatcaccaaaatgtgatccttggtcagtttacctaaatatcaatgtaattatgtttatctaatttaataatttgctgaaatcttccttattttttactttttatgaagcttttagccatttatattagatggtgat

SEQ ID NO: 211 - DNA, 401 bp, Homo sapiens
tcattttgttgcccattcagagagcttgtaagcttgggctctgccgcttttgcaaaagccaaggtaaagccaggatcgctgccaagttgtttgcactctttggagttctagttagctcagggcctgactgtatttttcatccatcttttctgaagtgtctttgggcagtatgtagttatttattacaaaattatattcacstaaatgccaaccatctacaaaaacaatgagtaatttttctactttgaagatacacagatggggacaaaaaccctgttttggaattctgttctattcctcagtatccagaaagttactgacacagtaaaacaaggaaagttctaccctaagagccgccatcacttcaggccgctggtttgtcagccatctgttgcttctta

SEQ ID NO: 212 - DNA, 406 bp, Homo sapiens
  misc_feature (201)..(201): n= absent or G
ttgacatgtattagaattgactttatgtgacacaatggtgggcaagatattttcagtaatttattattttaatactacagggccctttggtgtcagaagagagttggtcaaaatctaggacctttaggggaagattgatgggggacttttcttaaaactaggaactagctgtgatataattgaataactattggacttagntcagaagacaaattttaagtactgattttctcactgacttggtgactttgggcaaatctgtcccttagtatcagttttctcacttataaacacagagtaatgatgattactttatcttttgagatgattataagcatgatgtgacaaaaaataacacaggaatttacactgctggcctaaaatgatctggtaattgctggccaaa

SEQ ID NO: 213 - DNA, 401 bp, Homo sapiens
gagcaggtgatcagttatatcaaatgctatcaataggttgataagatgagcctgagaattcacatttgtatggcaccaggaagtttaccagtgaccttgataaaaatacttccggccgggcatggtagctcacgcctgtaatcccagcactttgggaggccaaggtgggtggatcacctgaggtcagcagttcaagaacastctgtccaacatggtgaaaccccgtctctactaaaaatacaaaaattagctgggcgtggtggtggtgcctgtaatcccagctactcaggaggctgaagcaggagaatcgcttgaacccaggaggcggaggttgtagtgagtggaggtcgcaccactgcactccagcctgggtgacaagagtgagacttcatctcaaaata

SEQ ID NO: 214 - DNA, 401 bp, Homo sapiens
tacctcatagaagaaaatatttaaagctctttctgacttcatttgtttatatatgccatctttttttttttgtttttaaagaaacaagatctcactctgtcacccaggctggaatgcagtggcatgatcatagctcactgcaattttgaactcttaggctcaactgatcctcccgcctcatcctcccgagtagctaggccmacaggcatacatcaccatgcctggcttaatttttttgtagagacagagtctctctatgttgcccatgctggcttgaactcctggccttaagcaatcctcctgccttgccctcctaaagcactgggattacaggtgtaagccacgatgcccagcctgtatatgtcaacttagtcttaaggaatgttgtttgaattctgttt

SEQ ID NO: 215 - DNA, 401 bp, Homo sapiens
ttgttttggctggcgatcaccctgctcaggttcagaccttagttctgtttcaccatctgtggccagtggctccaatgttagtttagttctcctagcctttgtatggtaggcagaataatggtctccaaagatgtccatttcctaatccctgaagccttggtaatattttaggttacataatgaagaggagttaggttgcarttagagttgcggttgctaatcagctgaccttaaaataaagaggttatcctggattatctagttaggcccagtgtagtcataaggtttttaaaagtgagaaagtgaggcagaagagtcagtatcagagtgacaaagtgtgagaaagattcagcctgcacttgtggctttgatgatgggaggggggcccaagctaaggaatg

SEQ ID NO: 216 - DNA, 401 bp, Homo sapiens
ctcttatttaaaacattttaactttatcctttatcgtcaccacaataatgagctgttgttctttaaagcagtgaactaaatactctgttacacagagagccatgctcaacactgtgcttcgagaacacatgggctgcttcctttggttcaaaatctccccactggcgcattttaggtgttttgatcatgagtcaccaggakctctaaagcacttaactgagtctggggatttctaatctttctgccagttgtttgtagggaagtgctctgtgagctctacctctgaggctccatgctccctctggccctccctttaatagcttctcttccacggagatgcagtcaagtgctgaagcagcaaacagcactggaatttttgcccccacttttttgtcttccca

SEQ ID NO: 217 - DNA, 401 bp, Homo sapiens
atgagctgttgttctttaaagcagtgaactaaatactctgttacacagagagccatgctcaacactgtgcttcgagaacacatgggctgcttcctttggttcaaaatctccccactggcgcattttaggtgttttgatcatgagtcaccaggagctctaaagcacttaactgagtctggggatttctaatctttctgccarttgtttgtagggaagtgctctgtgagctctacctctgaggctccatgctccctctggccctccctttaatagcttctcttccacggagatgcagtcaagtgctgaagcagcaaacagcactggaatttttgcccccacttttttgtcttcccattgattaccatgttaacatgtcactctgtgcataaccctggcaaaga

SEQ ID NO: 218 - DNA, 401 bp, Homo sapiens
tagcacctagcatatgttgatcttataatagtgattaataagcagttaatgattgattaaagaacttatggtctgtctttgggattcatgtagataataggaaaggcaaagcagaaaaattcagttaattcagatgattctaataattattaaaatattttaaaatttccaactgcaaagaaaataattttttatagaacsattagacccagagaactcatacctgtaattagaagaaccctaagtcattgtagacagaagagatcctttcttttttacaagcacttgtgtcccagggacagtaataatattgtttaatatttctgcagcagtttacagtttaaagacactttcatggccgggtacaatggctcacgcctgtaatcccaagactttgggac

SEQ ID NO: 219 - DNA, 401 bp, Homo sapiens
  misc_feature (201)..(201): n= absent or T
tgccacccaacttttaagatccagctgagatttcacctcctccttacagactgttccagccctcattcgtttgcctgtctgctaaattcttagcagtgcacttataatctgttccacataatagtaccctcttttattgtttttattagttcaataatgataatgtgcttaagaacaaaaattgtgtctattctttttttnatgtgatagcacctagcatatgttgatcttataatagtgattaataagcagttaatgattgattaaagaacttatggtctgtctttgggattcatgtagataataggaaaggcaaagcagaaaaattcagttaattcagatgattctaataattattaaaatattttaaaatttccaactgcaaagaaaataatttttta

SEQ ID NO: 220 - DNA, 401 bp, Homo sapiens
  misc_feature (201)..(201): n= absent or G
gaaatgcctatacctgtgtgtatgtaatttgcaagctcttttgaaaatttttggaagacgaagtggttttattgtttctttatttttgaaactgcctcgctctgtcagccaggctggagtgcagtggcaccatcttggctcattgtaacctccacctgctgggttcaagcaatcctcccgcctcagccttccaagtagctnggactacaggcatgcaccatcatgtccgactaatttttgttgttgttgttgttattttttgtagagtcaggggttctggcatgttgcctaggctcgtattgaactcctgagctcaattgatctgcccaccttggcctcccgaagtgctgggattacaggtgtgaaccaccacactcggccaagacaaagtgttagtaatt

SEQ ID NO: 221 - DNA, 401 bp, Homo sapiens
gattctcataaggaacacgcaacttagatccctcacatgcgcagttcacaataggattcatgctcctatgagaatctaatgacacctctgatctggcaggaggcggagctcaggcagtcatgctctctcgcccaccgctcacctcctgccatgcagcccagtttctaataggccattgacaggtactggtccgcagccctrgggttagggacccctgttgtagagcatataaaaactgaagaaagtttcatagcatataaagattagtgcttggggtttctgacagtgacaaaacaatttttttcctttggaatttaggatatacttcttatcctgtcctttttcgcctcttgccctcaactccaatgcattttccttacataaattaaaagggaccatca

SEQ ID NO: 222 - DNA, 401 bp, Homo sapiens
tcttgaaaatcctgagacatattcaaatacatagtttttacttacaaaaatacttataggcctggtgcggtggctcatgcttgtaatcccatcactttgggagaccgaggtgggcaaattgcctgaggtcagtctggccaacatggtgaaaacccatctctactaaaatacaaaaaaaaaaaaaaaaaaaattagccaggyctggtggatcacctgtaatcccagctactcgggaggttgaggcaggagaatcacttgaatccaggaggcggagttgcaatgagctgagatcacaccactggactccagcctggtgacagagtgagactctgtcttaaaacaaaacaaaacaaacaaacaaacaaaaaacatataaagatgctctttactatccatttcca

SEQ ID NO: 223 - DNA, 401 bp, Homo sapiens
tcctgagacatattcaaatacatagtttttacttacaaaaatacttataggcctggtgcggtggctcatgcttgtaatcccatcactttgggagaccgaggtgggcaaattgcctgaggtcagtctggccaacatggtgaaaacccatctctactaaaatacaaaaaaaaaaaaaaaaaaaattagccaggtctggtggakcacctgtaatcccagctactcgggaggttgaggcaggagaatcacttgaatccaggaggcggagttgcaatgagctgagatcacaccactggactccagcctggtgacagagtgagactctgtcttaaaacaaaacaaaacaaacaaacaaacaaaaaacatataaagatgctctttactatccatttccatcacccacc

SEQ ID NO: 224 - DNA, 401 bp, Homo sapiens
cacttagacatggatgtctatgcatagacatggatgtgcaggaggtgaatggcacttcagaggacaggttcctgtcagcctctttggattcacgtcccagctctacaactttcagcctgggtgatctggagcaagttactaaatcattatgtgtttttattgcttcacctataaaatggcacctgcttcatagagtgggcrcaagtattaaattagattttatacgtaagcattcagcacagtgcctggtaaactgtcaaaaaatggtggccgtttacattttttctgcataaaagttttgaaggacttcagttaattcagaacataaaagtgggtcatgaaataaaagtagctctatacttggaaggcaagaaaatttgaatctaattctattttttcta

SEQ ID NO: 225 - DNA, 401 bp, Homo sapiens
ccttggccgttcagtcagaggggtccattcggtcagttgaggggcctagaattttatttttggtttacaaaatcattccaagatcctctttagaggaaaaatttatagagattagtgggaatgatgaggagaactcaatcttgagagctcaatcaaaaggagatgtttaaatatctttttaagttggtattggtaaagtgmtttgaagacagaaagaatgtaatacatgtctggtgtctgcttgtcctataattgtcggaagggcctcaatgatgaaataagggaggctgccatgacacttgagtcttggtgagaggagctagtgtgtccacatttatcaagatcacctgcaggagtttgggctggccccctcttattagtagtttctctgttttttaaac

SEQ ID NO: 226 - DNA, 401 bp, Homo sapiens
aaaatacttataggcctggtgcggtggctcatgcttgtaatcccatcactttgggagaccgaggtgggcaaattgcctgaggtcagtctggccaacatggtgaaaacccatctctactaaaatacaaaaaaaaaaaaaaaaaaaattagccaggtctggtggatcacctgtaatcccagctactcgggaggttgaggcagragaatcacttgaatccaggaggcggagttgcaatgagctgagatcacaccactggactccagcctggtgacagagtgagactctgtcttaaaacaaaacaaaacaaacaaacaaacaaaaaacatataaagatgctctttactatccatttccatcacccaccgtcagtggtccagacacactttctccatgcttccgct

SEQ ID NO: 227 - DNA, 401 bp, Homo sapiens
gtggctcatgcttgtaatcccatcactttgggagaccgaggtgggcaaattgcctgaggtcagtctggccaacatggtgaaaacccatctctactaaaatacaaaaaaaaaaaaaaaaaaaattagccaggtctggtggatcacctgtaatcccagctactcgggaggttgaggcaggagaatcacttgaatccaggaggyggagttgcaatgagctgagatcacaccactggactccagcctggtgacagagtgagactctgtcttaaaacaaaacaaaacaaacaaacaaacaaaaaacatataaagatgctctttactatccatttccatcacccaccgtcagtggtccagacacactttctccatgcttccgcttaagcttctcagcaccaagtatt

SEQ ID NO: 228 - DNA, 401 bp, Homo sapiens
ctttgtggcttcatctccagccacactggacagccacccccagtttctgcacatgcactgctctcttgtgttcccggaccaaactgagggtcaggctgctattttttgctgccccaaaacgagatgcagatgaactgggaagagactttttatttctataaccagttatatagggagaaggcctggaaattattgccagamcaactcaaaattacaaagttttccagagcttatataccttctaaactatatgtttacgtgtaagtgtgcatttctctaaagacataagtgattaacttcttttaatccataactaaggtccgagtcttgaagaccttcctcttgagcctcagtaaatttacttaatctaaatgggtccaggtgctggggtgattaccctt

SEQ ID NO: 229 - DNA, 401 bp, Homo sapiens
attggaagaggagaatcaatgatgaagatgaaagaaatgtggagattgggggtaaaggaagaagctgataggcagagattttaaaaatggtcatgccttgatccttgcaagtctttggttcagaaatgagcttcagttggagagcaggacactgttgtatgaggttgaagacagagtctaggttggaaggggacaggtagrtaggtctggttggattaatggaattgggggctcaggggacaaatgagttaagattggcatttgggagccttgccaagagatagaaaacatttgccagaaatttaagcatactgtcttttttatagtcagaaaattcagtcactcgtaagttgggactgttcacttgtctgaatgttttagatttaaagaaaaaatatagc

SEQ ID NO: 230 - DNA, 401 bp, Homo sapiens
aaagaaaggaaggaaggaagaaaaaaaaggaaggaaggaaggaaaagaaaagagggagggagggaaggaaggaaagaaggaaggaaggagaaagaaaagtagatctaacttattttgggcatgtgtattagtttactagtgttagcaatggcaaatccgtcgggtctgcagaaactctatttttgccttcttggaggaaakaattctgctgaggggcataaggcagagagactgaggcaacttttagagcaggagtgaaagtttatcaaaaagttatagagcaggaatgaaaggaagtaaagtacacttgcaagagggccaagtgtacctgagagatccaagtgcactgtttggcccttgacttgggggttttacacattggcatggtgccaggatttctg

SEQ ID NO: 231 - DNA, 401 bp, Homo sapiens
attgaacctttttctcctagatttttcttttttccctctcatttagttcttttttagtcttgatttccccacggagagtctcatctattcacatattctcattttttcctttttaaaatacatcttcctgcttaatgatggggatacaattgaaaaataataaaacacgtcttcttcagggattcttttttatttataatrgctactctaaagactcactaaatacaatgcaatatctggaccaccttaagattgctttctaatgattttgtttacttagggttcacattttcttgtttcattaaatgtctagtaattttttattacatattgaatagtgtcaatggcacatggtagagatgctgaattaaaaaaaactctgtaaaatgttgatttttctc

SEQ ID NO: 232 - DNA, 401 bp, Homo sapiens
aaaataccttgaaatacttactgacattatagaaatttagcccttcactctgctgatgtttatagttaagtgtcagaaatacttttatacagaagaccttgtatggttcctttgtgtgagtggacagaatttgtggagcaaagacctggaatccagcatatgagaatgtgcaataattgttcaaatgaataagcttctcaratttggcctttgtataattaaaatcagagtgctgaagtgttgcatattcctcattcttctcattcttgcaagtctctctctctctctctctctatatatatatatacatatatacatatatacatatatacacatatatacatatatacacatatatacatatatacacatatatacatatatacacatatatatacata

SEQ ID NO: 233 - DNA, 401 bp, Homo sapiens
agaatacaactggattttcagatttgcttctgcattcagtcagttgtaatagcacaagtcatgtagcctgtggaaaactctgctgtacactcatgagagaagtggagtgaaaatggcatataacatattatgatgaaatagttttgactctgaaggcctcctgcaagggtatcagggatttctaggtgtacccatatcacrtcttgagaacattaatcttgcgtttttcaggaactggagaggaatagtttaggagtccacagaaggtagaaagtggagctgttggaattgggcagcaagtttcttaagatagatctaggtcacaggaggggaatgttctggccaggcatatttgactggccacattatcagatgccttggttatgtgcggattctaccgt

SEQ ID NO: 234 - DNA, 401 bp, Homo sapiens
ttccatcatctggtgattgcgttttccattgaggattgtttacattttcttggtcctttgtatttcaagtagttgtggcttgcatcctggacattatgggtgttatattgtgtagactcttttatcctctgaagaatgttgatatttttgttttggctggcgatcaccctgctcaggttcagaccttagttctgtttcacmatctgtggccagtggctccaatgttagtttagttctcctagcctttgtatggtaggcagaataatggtctccaaagatgtccatttcctaatccctgaagccttggtaatattttaggttacataatgaagaggagttaggttgcaattagagttgcggttgctaatcagctgaccttaaaataaagaggttatcctgga

SEQ ID NO: 235 - DNA, 401 bp, Homo sapiens
agagccacaaaaacaattcccaagccaattaaattcaacttttaaaaaggaatttcctaatataccatagagttggtgagaaggcaatgaatgggtcccacaagctttcatgtagccttatgggaagagtaaaggttaagctgtgtcatggttgtcaactgggcaaagccactgaaaggcaggactctctattagttgacrtaacaaaatattaataactagtgttatgaattagttgcagtatgagctgaggtatgaaagcatgaattttagacctgacactatccaggagggaaaaaagtggatgtttctgtactgatgttaatcaaaggttaaaaatcaaatgacattttgaggaaaacaaacctaaacaactcattaatggccacacaacttaaatt

SEQ ID NO: 236 - DNA, 401 bp, Homo sapiens
aggcatgtgctaccatgcctggctaatttttatatttttaagtagagatgaggtttcaccatgttggccaggctggtctcaaactcctgatctcaagtgatccgcccaccttggcctcccaaagtgctgggatttcaggcgtgagccacctggcctggactgtaattgaggatttttctgtgtcatattctcaactgttgytggtgtgctacagaaagaggaggaaattttttttaatctctgaggcgagtaaaggaaaccagaatactacaggacacctaattttttcaatcttcatgaaaatgcaagctgtgaatttgaggtttggtatcgtgaagccagagtctgtacagataattcgcagcaattaatgaccacccttcttaataatcttccatcag

SEQ ID NO: 237 - DNA, 401 bp, Homo sapiens
gtaatcctagcactttgggaggccaaggcaggaagattgcttgaggccagcagttcaagaccagcctgggcaacatagtgagagcctgtctctacaaaaaaattaaaaattaaaaaaaaaaattagtcaggtgtgatggtatgcacctgtggtcccagctgcttgagaggctgaggtgaaaggatcacttgagcctgggcwaagtggaagtgagctgtggtcatgccactgcactgcagcctgggcaagagagtgagaccctatctcaaaaaaaaaaaaaaaaaaagagatcagaaaggtctttttctatagaatgttccacacaagagacagctttgcagggccatttcaaaataggtctaagaaatatattttggggtaaaatacctttatttctttca

SEQ ID NO: 238 - DNA, 401 bp, Homo sapiens
ggattgatctgctttctcaagctttgccccgggcctaaccaggtcagcctgggaccagcccgtggggtttgactatacctggaacagatggttaatctattggcttgctataatgtaatttccatttggctggcagtagggaaaggaaggtacttcctgtaagctacacactgattttcatccaggtgttcacacataccrggttttatgaaagagagcttgaccctcgcattcctgattagcattttgttagtgtgaaagtaaggtatagacacagagacaggtataatcacaaaatggttggagtcttttattgtgtccttttcttagagcaaatttaatagaggagtttgattagcacctaagacttgcttaaaactgagttactaattctttttcca

SEQ ID NO: 239 - DNA, 401 bp, Homo sapiens
tccttccctccctccctcctttccttttccatcctccccctcacctttcctttttctgtaaacttttccatagcaaatagagtaattccaaatcattttttggaacattcttattagtgttcattcagcttccctttccactgaaatgaatttattgagtacttggagtatttcaaaccctatgctttgtacagagaagasagtgctaaataggaaaccctctcagtgttagagggaaaggaagatgatgtggagggaaggagtctcttactctgaagcattttcaaaatcaacattaaagagtgaaccaacatttacctcctttcttctttcatcttcttatttcatagctagagagctgctgtgcgtttgagacctaagtggtgcaattaaatcaattt

SEQ ID NO: 240 - DNA, 401 bp, Homo sapiens
ttttgttttttactccagatgtgttgatagaggttaatataagaaatgtttgtgttggcataatgcaaaggttatttttgattctgaacccatagcagtttctaaccggtgttcgtcagtttgtgcttgcttttatccttgaggttaaggattgctcaccaagcctttgattactaggtacattgcagaataaataaaatsgttgctagtgtatactctgtattaatctgtccacagcagcattgtcagtgatctcaaggttctctgtagacattagtattgggttatggcatgctaaaatagagataattgagactataaagttccaagttgaagttatcaataacaaccctaaaactatcttccttttctttccttctaaaataagacatatggtaatc

SEQ ID NO: 241 - DNA, 401 bp, Homo sapiens
tcatcccccacatgtggcaagacaagttggccctttcttacccagaggtcttttgtgtgactgcatctttctcctccgttctccattgtgtgctttccattttgtctttagtgcctatactgttaggtgttttcttcactggcattcacaaatttaagccattgctgcctcattagccttgtattttgtgtgcatatcatrtatccagacctgtatgttcgctttaagcattcttatatcacactgtctcctcatctaccatatggtaaatgttaaaactccacatttgtctgcatcagggaaaatgcatgggcacacatcctccctccctccctctctgctctcctcccttccttcaggcctcttagcattgtttgttttcccatttctgatactactac

SEQ ID NO: 242 - DNA, 401 bp, Homo sapiens
tgaaacagttgttatggaggcctgcgttagtgagatctggcttgccacacttgtgttacccactctttccagagtatactttcttcccttcttcaccttttcaaatactcatctttttaggccctcttcaggttttctgcatgtttccttataatatcttcaacctctagtcagaatttgtttccttccctttgttcccaytgctttattttcattgttaggacatgacttacagcctgatgtaagtttctgttcattgtataaacctctgcctttcccagtttattgcagatcctttagtaactaggattgtaacatatttatcttagtatacttggcagggtgccttgtacagtaggtgctcagtaactactggattgaatttgtgtttgttttaggta

SEQ ID NO: 243 - DNA, 401 bp, Homo sapiens
agcagcgtactgctggctctgcaccggacgccgcgcaagacgtcccgcatgcacctcttcatccgacacctcagcctggccgacctggccgtggcattcttccaggtgctgccgcaaatgtgctgggacatcacctaccgcttccgcggccccgactggctgtgccgcgtggtgaagcacctgcaggtgttcggcatgttygcgtcggcctacatgctggtagtcatgacagccgaccgctacatcgcggtgtgccacccgctcaagactctgcaacagcccgcgcgccgctcgcgcctcatgatcgcggccgcctgggtgctgagcttcgtgctgagcacgccgcagtacttcgtcttctccatgatcgaggtgaacaatgtcaccaaggcccgcgactg

SEQ ID NO: 244 - DNA, 401 bp, Homo sapiens
aactgtgaaaaataaaataaggtgctgcaacacatttttttcttgactgtaagctgttatttggcataatatctcaggtcttctctttagtcaagaaaaggaaaacttcccttcccggaatactttttcagtttctcttcttctgaaacagacaggcaggtagattccttccaatctgaaatattgttttgagatatgtgrcgtccatttctgggtacataacattgagaaaatttagcaaccagacagatgaaacttctcagcctaaaccgcagagaataagaccatgtatttgcctagtgcagaactagcacccagatctcatgtttccccagcccattttctactgtctcatctcccaatacatttaaaaggagaaaatacaactgggtagggtgata

SEQ ID NO: 245 - DNA, 401 bp, Homo sapiens
ttgagatatgtggcgtccatttctgggtacataacattgagaaaatttagcaaccagacagatgaaacttctcagcctaaaccgcagagaataagaccatgtatttgcctagtgcagaactagcacccagatctcatgtttccccagcccattttctactgtctcatctcccaatacatttaaaaggagaaaatacaactsggtagggtgatatgcacttttttttgtgagctgttctcagaaataacattcaaattgaattgttttgcttgggggtacatatcaacattttgaagcaagatctataggttctgaggttcttactttggaaatggatttagaaaaaaatgggttcatcttagttccaaaccaaaaagctttagtttttgaactatcaaaga

SEQ ID NO: 246 - DNA, 401 bp, Homo sapiens
aacctcccaagtagctgggactataggcacacaccaccatgcccagctaattttttgtattttttttttctttttagagtagagataggggtctccctatgttgcccaggctggattatacatgaatttttaaaaatgaaagttacactgaatgtgcctgcctgtcctgcctcccctttcacctcctccaccccttccacycgagacagcaagatcaacccctcttctgcctcttcctcctcagtctactcaacctgaagatgagggtgaagacctttatgatgatccacttgcacttaatgaataggaagcactttctattatttttaaataacgttttcttttctctagcttactttattgtaagaatacagtatatcatatataatatgcaaaatatg

SEQ ID NO: 247 - DNA, 401 bp, Homo sapiens
atatgtatgcatctggccattctatgtatcatgtgtcaatcaatcatctatctatctgtctatctatctatctatctatctatctatctatctatcatccatctatctgtctctcgctggttgtgctggatgccatggggcctggaaagcaggaaaaaaaaatgttcattgcagattgtagaaccagtccctttgtttaayccatatagttttaaacatgtttttgacttaatttaactggttttatatacaaaggaaagcaggactattacatatgaggcactactcatatgcctcactggacctgctattaaattaccccatagagagtaaaataattgtggtcttaaaatatgaaaaagaaaacacaacagacaatattttatgtggcaccttgtgct

SEQ ID NO: 248 - DNA, 401 bp, Homo sapiens
tttacatgtccatccctgtgggcaggaaaagagtgaaaacagccttatgtggatcgcatgaaatggattcctcacaggaaagaatgctcctgttgctagaaaaggagggtatgctaagctggcaaaaataacagatatttacttcacatatgaaaaccaacctgttgatctcagacttgcaatggatggctgaatttcatyctagcctttctttgcataagtgaccggggaagaagtactgactttacttttatcatttacagtgattttttttctgtatatgctagttaattaaactgaataaaaggaattcctatattatgataatttagtctcagtaatagccaataaatatttctggaaagaagtacccagcccctgtgtgggtgctattattgaat

SEQ ID NO: 249 - DNA, 401 bp, Homo sapiens
tccctgtcttagaagaaaagttttcaactttttaccattaagtatgatgttagctataggctagtgatctatggcctttattgtgttgaggtacattccttctatacctaatttgttgagaatttttatcatgaaagtgtgttgaattttgtcaacttctttttctgcatctatcaagatgatcgatcatatggcttttayttttcattctgataatatggtgaatcattttattggtttctgtatgtggaaccatcctaggcaagtcagattttggatttcctcctttatgttccattctgtaacatgttaatgggaaccggaattcagatcagaataacagcttaggaaccaaagcaggtatatatatatgtgtgtgtgtgtgtgcgtgtgtgtgtata

SEQ ID NO: 250 - DNA, 401 bp, Homo sapiens
tgtaacactgatcaaaatacgtttacaatgtccaagagaaagtccagagtaccttaagcaatccttttctactcttttaataaaatttggctttccttatacaaatctgactttaaaacaacctcgcagtgggggaaaaaagatatttttggccagtcaacatttcctctcaccttcagcatctcagttttcatgcttttmttgaccaataatatgtgaggaaccaaaaggaggccactgccagttgtaaagttaccattttgaaatgcaaggtgattgatagcttctaatagaactctaaactggccacaatgagcaggagctcattacaccccaggcacttgtactcctaggaggtaccatcccatctcctggacactgtttaaggctgcattttctga

SEQ ID NO: 251 - DNA, 401 bp, Homo sapiens
tttttgagaatattttctttttttccaaattattacatactcagatatactcttgaatctctcattcaacaagtccccaaactttgtccatgaaacctttcttctctttcttttctctataccccatcattctaatttacatcacgttaatctttttggattatatttacatatttaatttctcttccactttgctccaaytcaaattctttataacaaccacaagaacacgaaactcctgtaactagccaatgtagtaattagggtaggataggctatgtcctgtagtaacagagcaattctgacacctcagtggcttaacaaaaaaattatttctcactcatgcaaagtctaatgcaagttgagcagctttcccccaagcagtgactctgaggtccatg

SEQ ID NO: 252 - DNA, 401 bp, Homo sapiens
ttttattttgatctagatgtctgagagtatgaatgttcttagtgcaaataataaattgaatgctctcgaggataaaatttgaaaataattctatcttaagatgtctaacaaaatgaataaaaattataaactcttatgaatgaggttgtactctccaagtgtttcttgttaagaaccatagaaggacttcccttttagaartgctttggatattctaatacatttaatgccagggcataagctagtggttgttaagcttttctctccctcccacagcaccaagagacctaacattcacccatttgtagcagttgttttagaacagtgattgccaaggaggggaaaaatgaggaagcatagcagaatttctggggaactgtagaaagagagagcatattggg

SEQ ID NO: 253 - DNA, 401 bp, Homo sapiens
ttccacagctttgtgaacacagaatagtcccattgaaaagaaaatctttccgaatttgataaatgaataagtatctgattgttttaatgtatttcgttagaaatatttcatcgtttttgtctcattacttacttaataatgagttaaacattttcataaatgtcttataacttacaaacagaatctgggagtgctgaattrtgataaaggaactgctcaagttagaaatattacttttacttttctttgaactgttataaattatacagaaaaaataatacatggtatatatggaccataagtagcaggagctgtaatccaggttttgcatacattatcttatttaatcctcacgaatctaatgagatggattaaccactattttacacataaggatgcgg

SEQ ID NO: 254 - DNA, 401 bp, Homo sapiens
tgatgccaacactataattgccaaccccattgagaaaggaaagaaacatttgccctgacatcttccctccaggcagggctggccatgccactagtagcaaagaggagggatgtgttgagtcatctagtaagtccctgtgaagagtggatcctggcccatctgaacatctgaccagaaactagtggcagcagttgtagaacktggtatatgcatgtgcttctctttttatggaatcggaatcagggtgccccagaaagaaaacgagcccaattttaaaggggttaattgggtatcgtcttgattctttgtaagattggttaggtattcaggaatcaggctgaccaggcacaagtacctaccaacctttgtaaaatattctacactctacaatatcattcaca

SEQ ID NO: 255 - DNA, 401 bp, Homo sapiens
caccaccacgcccggctaattcttcatatttttagtagagacggggtttcaccgtgttagccaggatggtctcaaactcctgacctaaagtgatcagccagtctcgtcctcccaaagtgctgggattacaggcatgagccaccacacccagcaaagtggaacagaatagacagccgtaatggttccatgtatatttgggtrcttactatacaataaagaggtctccactaaaacaagggagaaggatggcataaaggagttgggaaatgcagaaaattatgctagattcatcttcttatatcacatcttagtagtagactccaaataaattaatgaagtaaatgtggaaggtaaattacaaacctgatagaaggaaaatgttattagagaacctatatgac

SEQ ID NO: 256 - DNA, 401 bp, Homo sapiens
gagctgcctaaggctgtgggagcccacctcttgcatcaatgtgccctggatgtgagacatggagtcaaaggagatcattttggaattttaatatttgactgccctgctggaatttggacttgcatggtgcctttagccccttcattttggccaatttctcccatttggaatgggtgcatttatccaatgcctgtactcccrttgtatcgaggaagtaactaacttccttttgattttacaggctcataggcagaagggacttgccttacctcagatgagactttggactgtgcacttttgagttaatgctgaaattagttaagactttaggggacttttgggaaagcatgattggttttgaaatgtgaggacatgaaatttgggaggggaccagggtggaa

SEQ ID NO: 257 - DNA, 401 bp, Homo sapiens
tctttcctctctttgagattgcctctttcttactcctgagcacaggagccgggcgggttttctgtcccttgccctggacagcactgcctggatggccgctgtccggcagctgctctttgtccacccaaaaagatgtccccacgactcagtagtaaccagacggtccccacggaccactgcggccaaatttccgccatcccygctgtgggaatcaggcttttcccgcagaaaaccccaggaatctagagaaaactccttaagtccctagtctccatagagaaaaccaggagacactccccccaaaccccgctgtgaatacaggcacagcagccactggggctgcaaagtgatgagtgcgttcttcccgtcgcaaacatagggtaataaatagcatgcatcaa

SEQ ID NO: 258 - DNA, 401 bp, Homo sapiens
aacatttcagtatgaatttaacttaaatattcttactgactataatactagcgataatgaaaaatacaatataaacactttatttttggtttgctatttcttatcttgcttgatcttagaagcctcttcatattgtccatcaaataaagaaattcagtctaattattgctttagcagaatttacactcaagtaataaaaayttcaattgtgcatagatatgttggtaattttcattctttgtgaataccatcttacccatggctcctgatcacctttgatagcagcatcttagcactaagtatgattaaataataacctgtaattgttttctggcataacaagagtgagaagatccaagtttatatttaataatcaaggaaaagtcagtgtttattgatta

SEQ ID NO: 259 - DNA, 401 bp, Homo sapiens
  misc_feature (201)..(201): n= absent or A
tgtctcttaaagggtactgtccaatataagccataactaaattaattaattcattatttgagttagagtagcatctcagtaacccagcactcgaagactgtcagtccttttaacaactctttgatagttcaaaaactaaagctttttggtttggaactaagatgaacccatttttttctaaatccatttccaaagtaagancctcagaacctatagatcttgcttcaaaatgttgatatgtacccccaagcaaaacaattcaatttgaatgttatttctgagaacagctcacaaaaaaaagtgcatatcaccctacccagttgtattttctccttttaaatgtattgggagatgagacagtagaaaatgggctggggaaacatgagatctgggtgctagtt

SEQ ID NO: 260 - DNA, 401 bp, Homo sapiens
ttccaattaaagcaaaatattcccaatttacatatgtgcaatgagaagagttttatggttaaatatgttggagaagtgctgtgtatgcatcccaccctctcctggtgatttatacataaaaaggacctgagaaacttcagaaaagaaacttacttaaccttgttcatcaatgttttccaaggttattttaccatggaaacyccccatttttttactttccccatggaatggtgatgaacatgtcacaagacaaggtgacagagcaggagcatcaccatcctgccattttaaagttcaccttgatcaaaaaccacctaaatccaaagggcatcagcctaatggctaaggccagaatgaccatgagccacaaataacatctcttaccagaaacattccaaacc

SEQ ID NO: 261 - DNA, 401 bp, Homo sapiens
ttatgcagtcttgtaggacacgttaaagatattgggcttgatctacaagaaagggaaaatgttgaaggaattttaacaagggaagggcataatcatttttgtatcttttaaaagagaatactttggctttatgtgcaaatgaatggaggagggtgagaacagatagagactcagttaagagaccatagcagaggacccgawaagctagagtatggtagggaagaagacatgcagagtcatggtcttgaggatgagtttgggagtattggaataatgaagtttacatctctattgccgaaatgaggattagtcgtgaccactcaaggcaggagccagccccacttatgagggagagaaggcagcagagtctggggacaggctcctagacaccttctggatca

SEQ ID NO: 262 - DNA, 401 bp, Homo sapiens
aacaacataaatgaattttttccaatagaaatgtaattgatttctgccccgtaggaaagaaaactccaagcattatgttttatgaaccaatagaaaaataataatcaatcttacatcttttagcaaaatcatttcagaattcttgactgcctgtgtgttactcttcttcagattctcccctgaacaggtcttaacatctcrttggttccatccttaattaataagctgaataaaactgtagcatgtgttcattttacatttgcaggagagtcatgactttatctttataaaatttatacatagcagccctgcgtggtctcaggggtctgcctctatctttgccacatcccatgctgaggtccatagctattctagctggtcctcactagtcctctgttcta

SEQ ID NO: 263 - DNA, 401 bp, Homo sapiens
ttctggttaaatgatttttaataagacgttaatctctttgtacaataagagtgcttatacccttttcataataattgtgtaaagacttatgattacactaggcacaggaaggtgttttcaataaaacaaagtgtccttccagttccctgctttgaagtagggtcttcaatcttcccatctccattgttcagtgcatatgtwtcacttaggataagctaagttatgctaaagtaacaagcaaacaacaaatctcagtggcttagagcaatcaagatctatttcttattcatgctactattcatcatgcatagctggggctttactccatgtgcttctcattcaggaacctaggtgagggggcttgatcatctggaatgtcaccagtcactgtagcagggaga

SEQ ID NO: 264 - DNA, 401 bp, Homo sapiens
aattatagatactgaaatctgaattttatacaatgttcatgtgtcagaaatattcattttgatttttctcaattatttaaaaatgtaaaaactattcttagctcataggacaaactaaaacatggatgagctagatttggcttgtgcatcatagtttgccaattcctgttctaaagtatgttaacaaatccacatatcttraatattactatttttcataataggtgagagcctatttttaactcccgttatgctgataaataagctactgatttcaccattatgttaattaacaaaatatctattgtcaatcagaagaaaaggtcaccaatattcttatagtagtcatctctggtgggtggggcttttctgataaaattctagctgcttccccattccct

(19) United States
(12) Patent Application Publication
(10) Pub. No.: US 2009/0298711 A1
(43) Pub. Date: Dec. 3, 2009

(54) VASOPRESSIN PATHWAY POLYMORPHISMS AS INDICATORS OF SUBJECT OUTCOME IN CRITICALLY ILL SUBJECTS

(75) Inventors: James A. Russell, Vancouver (CA); Keith R. Walley, Vancouver (CA); Hugh F. Wellman, Vancouver (CA); Nathan J. Markward, Baton Rouge, LA (US)

Correspondence Address: BROWDY AND NEIMARK, P.L.L.C.;624 NINTH STREET, NW, SUITE 300, WASHINGTON, DC, 20001-5303, US

(73) Assignees: THE UNIVERSITY OF BRITISH COLUMBIA, Vancouver, BC (CA); SIRIUS GENOMICS INC., Vancouver, BC (CA)

(21) Appl. No.: 12/162,066

(22) PCT Filed: Jan. 24, 2007
(86) PCT No.: PCT/CA2007/000111

§ 371 Date: May 8, 2009

Related U.S. Application Data
(60) Provisional application No. 60/761,328, filed on Jan. 24, 2006.
(60) Provisional application No. 60/795,127, filed on Apr. 26, 2006.

(51) Int. Cl. C40B 40/06; C12Q 1/68; A61K 38/22

(52) U.S. Cl. 506/16; 435/6; 514/12

(57) ABSTRACT

The invention provides methods, nucleic acids, compositions and kits for predicting a subject's response to treatment with one or more vasopressin receptor agonists to identify subjects having a greater benefit from treatment with vasopressin receptor agonist(s). The method generally comprises determining a vasopressin pathway associated gene polymorphism genotype(s) of a subject for one or more polymorphisms in the these genes, comparing the determined genotype with known genotypes for the polymorphism that correspond with an improved response genotype to identify potential subjects having an inflammatory condition who are more likely to benefit from treatment with a vasopressin receptor agonist and subsequent to treatment recover from the inflammatory condition. The invention also provides for methods of treating such subjects with vasopressin receptor agonists based on the subject's genotype.

### FIELD OF THE INVENTION

The field of the invention relates to the assessment and/or treatment of subjects with an inflammatory condition.

### BACKGROUND OF THE INVENTION

Arginine vasopressin (AVP) has both vasoconstrictor and anti-diuretic properties. AVP is synthesized in the hypothalamus and secreted from posterior pituitary gland, secreted into the circulation and binds to several receptors. AVP binds to vasopressin-specific membrane bound receptor AVPR1A on vascular smooth muscle (MOUILLAC B. et al. J Biol Chem (1995) 270: 25771-25777), AVPR2 in the distal convoluted tubule and collecting ducts in the kidney and AVPR1B pituitary receptors that modify adrenocorticotropin hormone (ACTH) production (ORLOFF J. and HANDLER J. Am. J Med (1967) 42:757-768). Binding to AVPR1A induces vasoconstriction. AVP has a very short half-life and is metabolized by leucyl/cystinyl aminopeptidase (LNPEP).

Under normal physiological conditions, AVP does not contribute much to the maintenance of blood pressure (GROLLMAN J Pharm Exper Therap (1932) 46:447-460; GRAYBIEL Am Heart J (1941) 21:481-489; and WAGNER, J Clin Invest (1956) 35:1412-1418). However, when blood pressure falls, AVP is fundamental to the response to hypotension as AVP is released from the posterior pituitary and causes arterial smooth muscle to contract (vasoconstriction) (WAGNER, J Clin Invest (1956) 35:1412-1418; AISENBREY J Clin Invest (1981) 67:961-968; and SCHWARTZ Endocrinology (1981) 108:1778-1780). If AVP is not secreted by the posterior pituitary in response to hypotension, then blood pressure remains low or falls further as a result of inappropriate vasodilation.

Critically ill subjects with septic shock have been shown to have low serum AVP levels (LANDRY Circ. (1997) 95:1122-1125). Although AVP levels are initially high in septic shock, they fall within hours (GOETZ Proc. Exp. Biol. Med. (1974) 145(1):277-80; WILSON Surg. Gynecol. Obstet. (1981) 153(6):869-72; (MORALES D. et al. Circulation (1999) 100(3): 226-9); and ERRINGTON J Physiol (1971) 217(1): 43P-45P). Indeed, septic shock develops in part because there is a defect in the baro-receptor-mediated increase in AVP secretion (LANDRY Circ. (1997) 95:1122-1125). AVP can be administered to subjects who have septic shock who are not responding adequately. It has been reported that AVP increases blood pressure, decreases need for vasopressors such as norepinephrine, and increases urine output (LANDRY D W et al. Circulation. (1997) 95:1122-1125; HOLMES C L et al. Int. Care Med. (2001) 27:1416-1421). In a small, proof of concept randomized controlled trial of norepinephrine (NE) versus AVP in subjects with severe septic shock, it has been shown that AVP spared NE use, maintained mean arterial pressure and cardiac index, and improved measures of renal function including increased urine output and creatinine clearance (PATEL B M et al. Anesthesiology (2002) 96:576-582). Blood AVP levels were also found to be very low (1.3+/−0.9 pg/ml) (HOLMES C L et al. Int. Care Med. (2001) 27:1416-1421; and PATEL B M et al Anesthesiology (2002) 96:576-582). Several other studies have also shown that AVP increases blood pressure in septic shock (LANDRY D W et al. Circulation (1997) 95:1122-5; MALAY M B et al. J Trauma (1999) 47(4): 699-703; GOLD J A et al. Crit. Care Med. (2000) 28(1): 249-52; and MORALES D L. et al. Ann Thorac Surg. (2000) 69(1): 102-6).

Vasopressin is commonly used after cardiac surgery as studies have shown that AVP levels are lower after cardiac surgery compared to baseline. In addition, AVP infusion has been demonstrated to increase blood pressure after cardiac surgery (ARGENZIANO J Circulation (1997) 96(9 Suppl):II-286-90; ARGENZIANO J Thorac. Cardiovasc Surg. (1998) 116(6):973-80; CHEN Circulation (1999) 100(19 Suppl):II244-6; and ROSENZWEIG Circulation (1999) 100(19 Suppl):II182-6).

Arginine vasopressin (also known as antidiuretic hormone or ADH) is encoded by the AVP-neurophysin II gene (AVP) which contains three exons and maps to chromosome 20p13. AVP is synthesized in the hypothalamus as a precursor polypeptide (prepro-AVP-NPII) and undergoes post-translational processing to yield three functional peptides: AVP, NPII, and copeptin (Entrez Gene; http://www.ncbi.nlm.nih.gov/entrez). The AVP-NP11 complex is transported along nerve axons to the posterior pituitary where it is secreted into the bloodstream or directly into the brain. In addition to its vasoconstrictor properties, AVP acts to maintain fluid homeostasis by signaling through AVPR2 receptors in the collecting ducts of the kidney (BIRNBAUMER M Trends Endocrinol Metab (2000) 10:406-10) and plays a role in pH regulation (TASHEVIA Y et al Plufgers Arch (2001) 442(5):652-61. Furthermore, AVP is thought to be involved in cognition, tolerance, adaptation as well as complex sexual and maternal behavior (YOUNG W S et al Neurosci (2006) 143(4): 1031-9).

A representative human AVP mRNA sequence is listed in GenBank under accession numbers NM_{—00490} (633 bp). NM 00490 contains AVP rs1410713 but not rs857242.

Human arginine vasopressin receptor 1A (AVPR1A) is also known as the V1a vasopressin receptor (V1aR); SCCL vasopressin subtype 1a receptor; V1-vascular vasopressin receptor; antidiuretic hormone receptor 1A; and vascular/hepatic-type arginine vasopressin receptor. AVPR1A maps to chromosomal region 12q14-q15. The protein encoded by this gene acts as receptor for arginine vasopressin (AVP). This receptor belongs to the subfamily of G-protein coupled receptors which also includes AVPR1B, AVPR2 and OXTR. AVPR1A agonist binding increases intracellular calcium concentrations by signaling through the phospholipase C cascade (OMIM: 600821). The downstream effects of this signaling cascade include cell contraction and proliferation, platelet aggregation, release of coagulation factors and glycogenolysis. AVPR1A has been investigated for associations with social behaviors, including affiliation and attachment (YOUNG L J et al Nature (1999) 400(6746):766-8) as well as essential hypertension (THIBONNIER Met all Mol Cell Cardiol (2000) 32(4):557-564).

A representative human AVPR1A mRNA sequence is listed in GenBank under accession number NM_{—000706} (4154 bp). The NM_{—000706} sequence contains AVPR1A SNP rs3803107 (and rs1042615), but not rs1495027 or rs10877970.

Homo sapiens leucyl/cystinyl aminopeptidase (LNPEP) is also known as AT (4) receptor; angiotensin IV receptor; insulin-regulated aminopeptidase; insulin-responsive aminopeptidase; otase; oxytocinase; placental leucine aminopeptidase; and vasopressinase. LNPEP maps to chromosomal region 5q15. The LNPEP gene encodes a metalloproteinase that cleaves polypeptides such as vasopressin, oxytocin, lys-bradykinin, met-enkephalin and dynorphin A (Entrez Gene: www.ncbi.nlm.nih.gov/entrez). LNPEP also catalyzes the conversion of angiotensinogen to angiotensin IV (AT4) and is thought to play a role in memory processing by acting as a receptor for AT4 (LEW R A et al J Neurochem (2003) 86(2):344-50. LNPEP also plays a role in the maintenance of pregnancy (NORMURA S et al Biochim Biophys Acta (2005) 1751(1): 19-25).

A representative human LNPEP mRNA sequence is listed in GenBank under accession number NM_{—005575} (4470 bp). The NM_{—005575} sequence does not contain the LNPEP SNP rs18059.

Homo sapiens leukocyte-derived arginine aminopeptidase (LRAP) is also known as endoplasmic reticulum aminopeptidase 2; (ERAP2). LRAP maps to chromosomal region 5q15, immediately upstream of LNPEP. The longest annotated transcript of LRAP (NM 022350) has 18 exons and is predicted to encode a protein of 915 amino acids (aa). LRAP is localized to the endoplasmic reticulum (ER) of the cell where it functions to cleave antigenic peptides greater than nine aa for presentation to major histocompatibility complex 1 (MHC-1) molecules (TANIOKA T et al J Biol Chem (2003) 278(34):32275-83).

A representative human LRAP mRNA sequence is listed in GenBank under accession number NM_{—022350} (3356 bp).

Genotype has been shown to play a role in the prediction of subject outcome in inflammatory and infectious diseases (MCGUIRE W. et al. Nature (1994) 371:508-10; NADEL S. et al. Journal of Infectious Diseases (1996) 174:878-80; MIRA J P. et al. JAMA (1999) 282:561-8; MAJETSCHAK M. et al. Ann Surg (1999) 230:207-14; STUBER F. et al. Crit Care Med (1996) 24:381-4; STUBER F. et al. Journal of Inflammation (1996) 46:42-50; and WEITKAMP J H. et al. Infection (2000) 28:92-6). Furthermore, genotype can alter response to therapeutic interventions. Genentech's HERCEPTIN® was not effective in its overall Phase III trial but was shown to be effective in a genetic subset of subjects with human epidermal growth factor receptor 2 (HER2)-positive metastatic breast cancer. Similarly, Novartis' GLEEVEC® is only indicated for the subset of chronic myeloid leukemia subjects who carry a reciprocal translocation between chromosomes 9 and 22.

### SUMMARY OF THE INVENTION

This invention is based in part on the surprising discovery that vasopressin pathway SNPs from AVP, AVPR1A, LNPEP and LRAP are predictive or indicative of subject outcome, wherein subject outcome is the ability of the subject to recover from an inflammatory condition based on having a particular AVP, AVPR1A, LNPEP or LRAP genotype as compared to a subject not having that genotype.

This invention is also based in part on the surprising discovery of vasopressin pathway SNPs having an association with improved prognosis or subject outcome, in subjects with an inflammatory condition. Furthermore, various vasopressin pathway SNPs are provided which are useful for subject screening, as an indication of subject outcome, or for prognosis for recovery from an inflammatory condition.

This invention is also based in part on the identification that the particular nucleotide (allele) or genotype at the site of a given SNP may be associated with a decreased likelihood of recovery from an inflammatory condition (‘risk genotype’) or an increased likelihood of recovery from an inflammatory condition (‘decreased risk genotype’). Furthermore, this invention is in part based on the discovery that the genotype or allele may be predictive of increased responsiveness to the treatment of the inflammatory condition with vasopressin receptor agonist (i.e. “adverse response genotype” (ARG) or “improved response genotype” (IRG)). The vasopressin receptor agonist may be vasopressin. The inflammatory condition may be SIRS, sepsis or septic shock.

This invention is also based in part on the surprising discovery that AVP, AVPR1A LNPEP and LRAP SNPs alone or in combination are useful in predicting the response a subject with an inflammatory condition will have to vasopressin receptor agonist treatment or vasopressin treatment. Whereby the subjects having an improved response genotype are more likely to benefit from and have an improved response to vasopressin receptor agonist treatment and subjects having a non-improved response genotype are less likely to benefit from the same treatment. Furthermore, there are provided herein AVP, AVPR1A LNPEP and LRAP SNPs and SNPs in linkage disequilibrium (LD) thereto, which are also useful in predicting the response a subject with an inflammatory condition will have to vasopressin receptor agonist treatment or vasopressin treatment.

In accordance with one aspect of the invention, methods are provided for obtaining a prognosis for a subject having, or at risk of developing, an inflammatory condition, the method including determining a genotype of said subject which includes one or more polymorphic sites in the subject's vasopressin pathway gene sequences or a combination thereof, wherein said genotype is indicative of an ability of the subject to recover from the inflammatory condition.

In accordance with a further aspect of the invention, methods are provided for identifying a polymorphism in a vasopressin pathway gene sequence that correlates with prognosis of recovery from an inflammatory condition, the method including: obtaining vasopressin pathway gene sequence information from a group of subjects having an inflammatory condition; identifying at least one polymorphic nucleotide position in the vasopressin pathway gene sequence in the subjects; determining a genotypes at the polymorphic site for individual subjects in the group; determining recovery capabilities of individual subjects in the group from the inflammatory condition; and correlating the genotypes determined in step (c) with the recovery capabilities determined in step (d)

thereby identifying said vasopressin pathway gene sequence polymorphisms that correlate with recovery.

In accordance with a further aspect of the invention, a kit is provided for determining a genotype at a defined nucleotide position within a polymorphic site in vasopressin pathway gene sequence in a subject to provide a prognosis of the subject's ability to recover from an inflammatory condition, the kit including: a restriction enzyme capable of distinguishing alternate nucleotides at the polymorphic site; or a labeled oligonucleotide having sufficient complementary to the polymorphic site so as to be capable of hybridizing distinctively to said alternate. The kit may further include an oligonucleotide or a set of oligonucleotides operable to amplify a region including the polymorphic site. The kit may further include a polymerization agent. The kit may further include instructions for using the kit to determine genotype.

In accordance with a further aspect of the invention, methods are provided for treating an inflammatory condition in a subject in need thereof, the method including administering to the subject a vasopressin receptor agonist, wherein said subject has an improved response genotype in their vasopressin pathway associated gene sequence.

In accordance with a further aspect of the invention, methods are provided for treating an inflammatory condition in a subject in need thereof, the method including: selecting a subject having an improved response genotype in their vasopressin pathway associated gene sequence; and administering to said subject one or more vasopressin receptor agonist(s).

In accordance with a further aspect of the invention, methods are provided for treating a subject with an inflammatory condition by administering a vasopressin receptor agonist, the method including administering the vasopressin receptor agonist to subjects that have an improved response genotype in their vasopressin pathway associated gene sequence, wherein the improved response genotype is predictive of increased responsiveness to the treatment of the inflammatory condition with a vasopressin receptor agonist.

In accordance with a further aspect of the invention, methods are provided for identifying a subject with increased responsiveness to treatment of an inflammatory condition with a vasopressin receptor agonist, including the step of screening a population of subjects to identify those subjects that have an improved response genotype in their vasopressin pathway associated gene sequence, wherein the identification of a subject with an improved response genotype in their vasopressin pathway associated gene sequence is predictive of increased responsiveness to the treatment of the inflammatory condition with the vasopressin receptor agonist.

In accordance with a further aspect of the invention, methods are provided for selecting a subject for the treatment of an inflammatory condition with a vasopressin receptor agonist, including the step of identifying a subject having an improved response genotype in their vasopressin pathway associated gene sequence, wherein the identification of a subject with the improved response genotype is predictive of increased responsiveness to the treatment of the inflammatory condition with the vasopressin receptor agonist.

In accordance with a further aspect of the invention, methods are provided for treating an inflammatory condition in a subject, the method including administering a vasopressin receptor agonist to the subject, wherein said subject has an improved response genotype in their vasopressin pathway associated gene sequence.

In accordance with a further aspect of the invention, methods are provided for treating an inflammatory condition in a subject, the method including: identifying a subject having an improved response genotype in their vasopressin pathway associated gene sequence; and administering a vasopressin receptor agonist to the subject.

In accordance with a further aspect of the invention, methods are provided for administering one or more vasopressin receptor agonist(s) to a subject in need thereof, said subject having an improved response genotype in their vasopressin pathway associated gene sequence.

In accordance with a further aspect of the invention, methods are provided for treating an inflammatory condition in a subject, the method including: identifying a subject having an adverse response genotype in their vasopressin pathway associated gene sequence; and selectively not administering a vasopressin receptor agonist to the subject.

In accordance with a further aspect of the invention, methods are provided for selectively not administering one or more vasopressin receptor agonist(s) to a subject, wherein said subject has an adverse response genotype in their vasopressin pathway associated gene sequence.

In accordance with another aspect of the invention, there is provided a use of a vasopressin receptor agonist in the manufacture of a medicament for the treatment of an inflammatory condition, wherein the subjects treated have an improved response polymorphism in their vasopressin pathway associated gene sequence.

In accordance with another aspect of the invention, there is provided a use of a vasopressin receptor agonist in the manufacture of a medicament for the treatment of an inflammatory condition, wherein the subjects treated do not have an adverse response polymorphism in their vasopressin pathway associated gene sequence.

In accordance with another aspect of the invention, there is provided a use of a vasopressin receptor agonist in the manufacture of a medicament for the treatment of an inflammatory condition in a subset of subjects, wherein the subset of subjects have an improved response polymorphism in their vasopressin pathway associated gene sequence.

In accordance with another aspect of the invention, there is provided a use of a vasopressin receptor agonist in the manufacture of a medicament for the treatment of an inflammatory condition in a subset of subjects, wherein the subset of subjects do not have an adverse response polymorphism in their vasopressin pathway associated gene sequence.

In accordance with another aspect of the invention, there is provided a commercial package containing, as active pharmaceutical ingredient, use of a vasopressin receptor agonist, or a pharmaceutically acceptable salt thereof, together with instructions for its use for the curative or prophylactic treatment of an inflammatory condition in a subject, wherein the subject treated has an improved response polymorphism in their vasopressin pathway associated gene sequence.

In accordance with another aspect of the invention, there is provided a commercial package containing, as active pharmaceutical ingredient, use of a vasopressin receptor agonist, or a pharmaceutically acceptable salt thereof, together with instructions for its use for the curative or prophylactic treatment of an inflammatory condition in a subject, wherein the subject treated does not have an adverse response polymorphism in their vasopressin pathway associated gene sequence.

The method or use may further include determining the subject's APACHE II score as an assessment of subject risk. The method or use may further include determining the number of organ system failures for the subject as an assessment of subject risk. The subject's APACHE II score may be indicative of an increased risk when ≧25. 2 or more organ system failures may be indicative of increased subject risk.

The improved response genotype may be found at one or more of the following polymorphic sites: rs18059; rs27711; rs10051637; rs1410713; rs857240; rs857242; and rs1495027; or a polymorphic site in linkage disequilibrium thereto. The polymorphic site in linkage disequilibrium is selected from one or more of the following: rs2762; rs10051637; rs1477364; rs7731592; rs7736466; rs1363974; rs2351010; rs1423357; rs1544777; rs2161548; rs38032; rs38034; rs38041; rs27436; rs27306; rs27307; rs27397; rs27659; rs27711; rs27290; rs38030; rs27294; rs27747; rs39602; rs248215; rs27302; rs2278018; rs1559355; rs3734015; rs4869315; rs2247650; rs2549781; rs2549782; rs2161657; rs251339; rs187265; rs2548527; rs1056893; rs2548523; rs2255546; rs2255637; rs1019503; rs251344; rs1981846; rs10071975; rs7700332; rs38042; rs18059; rs9127; rs7972829; rs10784339; rs3803107; rs11836346; rs7308008; rs11835545; rs7959001; rs11832877; rs10877977; rs2201895; rs7302323; rs10877986; rs2030106 and rs18059; rs27296; rs27300; rs27613; rs27711; rs38033; rs38035; rs38036; rs38041; rs38043; rs716848; rs1216565; rs1230358; rs1363907; rs1974871; rs2042385; rs2113050; rs2113189; rs2161658; rs2255633; rs2255634; rs2287988; rs2548524; rs2548529; rs2548530; rs2548532; rs2548533; rs2548536; rs2548538; rs2548539; rs2548540; rs2549783; rs2549784; rs2549790; rs2549791; rs2549794; rs2549795; rs2549796; rs2549797; rs2617447; rs2910686; rs2927609 rs3797796; rs3849749; rs3849750; rs4360063; rs4869314; rs4869316; rs6556942; rs7713127; rs7716222; rs7719705; rs10044354; rs10051637; rs10058476; rs12516666; and rs12716486.

The improved response genotype may be selected from one or more of the following: rs18059CT; rs18059TT; rs27711GG; rs10051637GA; rs10051637AA; rs1410713AC; rs1410713AA; rs857240CC; rs857242CC; rs1495027CC; and rs1495027CT; or a polymorphic site in linkage disequilibrium thereto. The adverse response genotype which may be selected from one or more of the following: rs18059CC; rs27711AA; rs10051637GG; rs1410713CC; rs857240CT; rs857242AC; and rs1495027TT; or a polymorphic site in linkage disequilibrium thereto. The genotype of the polymorphic site in linkage disequilibrium may be selected from one or more of the polymorphic sites and corresponding genotypes set out in TABLES 1B and 1D.

The subject having one or more improved response genotypes may be selectively administered the vasopressin receptor agonist. The subject having one or more adverse response genotypes may be selectively not administered the vasopressin receptor agonist.

In accordance with a further aspect of the invention, methods are provided for selecting a group of subjects for determining the efficacy of a candidate drug known or suspected of being useful for the treatment of an inflammatory condition, the method including determining a genotype at one or more polymorphic sites in a vasopressin pathway gene sequence for each subject, wherein said genotype is indicative of the subject's ability to recover from the inflammatory condition and sorting subjects based on their genotype. The method may further include, administering the candidate drug to the subjects or a subset of subjects and determining each subject's ability to recover from the inflammatory condition. The method may further include comparing subject response to the candidate drug based on genotype of the subject.

The polymorphic site may be selected from one or more of the following: rs18059; rs27711; rs38041; rs10051637; rs1410713; rs857240; rs857242; rs10877970; rs3803107; and rs1495027; or a polymorphic site in linkage disequilibrium thereto. The method of claim 2, wherein the polymorphic site in linkage disequilibrium may be selected from one or more of the following: rs2762; rs10051637; rs1477364; rs7731592; rs7736466; rs1363974; rs2351010; rs1423357; rs1544777; rs2161548; rs38032; rs38034; rs38041; rs27436; rs27306; rs27307; rs27397; rs27659; rs27711; rs27290; rs38030; rs27294; rs27747; rs39602; rs248215; rs27302; rs2278018; rs1559355; rs3734015; rs4869315; rs2247650; rs2549781; rs2549782; rs2161657; rs251339; rs187265; rs2548527; rs1056893; rs2548523; rs2255546; rs2255637; rs1019503; rs251344; rs1981846; rs10071975; rs7700332; rs38042; rs18059; rs9127; rs7972829; rs10784339; rs3803107; rs11836346; rs7308008; rs11835545; rs7959001; rs11832877; rs10877977; rs2201895; rs7302323; rs10877986; rs2030106; rs1495027; rs10877962; rs1042615; rs16856; rs18059; rs27296; rs27300; rs27613; rs27711; rs38033; rs38035; rs38036; rs38041; rs38043; rs716848; rs1216565; rs1230358; rs1363907; rs1974871; rs2042385; rs2113050; rs2113189; rs2161658; rs2255633; rs2255634; rs2287988; rs2548524; rs2548529; rs2548530; rs2548532; rs2548533; rs2548536; rs2548538; rs2548539; rs2548540; rs2549783; rs2549784; rs2549790; rs2549791; rs2549794; rs2549795; rs2549796; rs2549797; rs2617447; rs2910686; rs2927609 rs3797796; rs3849749; rs3849750; rs4360063; rs4869314; rs4869316; rs6556942; rs7713127; rs7716222; rs7719705; rs10044354; rs10051637; rs10058476; rs12516666; and rs12716486.

The method may further include comparing the genotype determined with known genotypes, which are known to be indicative of a prognosis for recovery from the subject's type of inflammatory condition, or another inflammatory condition.

The method may further include obtaining vasopressin pathway gene sequence information for the subject. The genotype may be determined using a nucleic acid sample from the subject. The method may further include obtaining the nucleic acid sample from the subject. The genotype may be determined using one or more of the following techniques: restriction fragment length analysis; sequencing; micro-sequencing assay; hybridization; invader assay; gene chip hybridization assays; oligonucleotide ligation assay; ligation rolling circle amplification; 5′ nuclease assay; polymerase proofreading methods; allele specific PCR; matrix assisted laser desorption ionization time of flight (MALDI-TOF) mass spectroscopy; ligase chain reaction assay; enzyme-amplified electronic transduction; single base pair extension assay; and reading sequence data. The genotype of the subject may be indicative of increased risk of death or organ dysfunction from the inflammatory condition. The subject may be critically ill and the genotype is indicative of a prognosis of severe cardiovascular or respiratory dysfunction.

The genotype may include at least one of the following risk genotypes: rs18059CT; rs18059TT; rs27711GA; rs27711GG; rs38041GA; rs38041GG; rs10051637GA; rs10051637GG; rs1410713AA; rs857240CC; rs857242CC; rs10877970CC; rs3803107TT; and rs1495027TT; or a polymorphic site in linkage disequilibrium thereto. The genotype may include at least one of the following risk alleles: rs3803107T; and rs10877970C; or a polymorphic site in linkage disequilibrium thereto.

The genotype of the subject may be indicative of decreased risk of death or organ dysfunction from the inflammatory condition. The subject may be critically ill and the genotype is indicative of a prognosis of mild cardiovascular or respiratory dysfunction. The genotype may include at least one of the following reduced risk genotypes: rs18059CC; rs27711AA; rs38041AA; rs10051637AA; rs1410713CC; rs1410713AC; rs857240TT; rs857240CT; rs857242AA; rs857242AC; rs10877970TT; rs10877970CT; rs3803107CC; rs3803107CT; rs1495027CC and rs1495027CT; or a polymorphic site in linkage disequilibrium thereto. The genotype may include at least one of the following reduced risk alleles: rs3803107C; and rs10877970T; or a polymorphic site in linkage disequilibrium thereto.

Alternatively, the genotype of the polymorphic site in linkage disequilibrium may be selected from one or more of the polymorphic sites and corresponding genotypes set out in TABLES 1B and 1D.

The inflammatory condition may be selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects, subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglottitis, E. coli 0157:H7, malaria, gas gangrene, toxic shock syndrome, pre-eclampsia, eclampsia, HELLP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis. The inflammatory condition may be SIRS. The inflammatory condition may be sepsis. The inflammatory condition may be septic shock.

The vasopressin receptor agonist may be vasopressin.

In accordance with another aspect of the invention, there are provided two or more oligonucleotides or peptide nucleic acids of about 10 to about 400 nucleotides that hybridize specifically to a sequence contained in a human target sequence consisting of a subject's vasopressin pathway associated gene sequence, a complementary sequence of the target sequence or RNA equivalent of the target sequence and wherein the oligonucleotides or peptide nucleic acids are operable in determining the presence or absence of two or more polymorphism(s) or in their vasopressin pathway associated gene sequence selected from of the following polymorphic sites: rs18059; rs27711; rs38041; rs10051637; rs1410713; rs857240; rs857242; rs10877970; rs3803107; and rs1495027; or one or more polymorphic sites in linkage disequilibrium thereto.

In accordance with another aspect of the invention, there are provided two or more oligonucleotides or peptide nucleic acids selected from the group including of: (a) an oligonucleotide or peptide nucleic acid that hybridizes under high stringency conditions to a nucleic acid molecule including SEQ ID NO:1 having a T at position 201 but not to a nucleic acid molecule including SEQ ID NO:1 having a C at position 201; (b) an oligonucleotide or peptide nucleic acid that hybridizes under high stringency conditions to a nucleic acid molecule including SEQ ID NO:1 having a C at position 201 but not to a nucleic acid molecule including SEQ ID NO:1 having a T at position 201; (c) an oligonucleotide or peptide nucleic acid that hybridizes under high stringency conditions to a nucleic acid molecule including SEQ ID NO:2 having a G at position 201 but not to a nucleic acid molecule including SEQ ID NO:2 having a A at position 201; (d) an oligonucleotide or peptide nucleic acid that hybridizes under high stringency conditions to a nucleic acid molecule including SEQ ID NO:2 having an A at position 201 but not to a nucleic acid molecule including SEQ ID NO:2 having a G at position 201; (e) an oligonucleotide or peptide nucleic acid that hybridizes under high stringency conditions to a nucleic acid molecule including SEQ ID NO:3 having an A at position 201 but not to a nucleic acid molecule including SEQ ID NO:3 having a G at position 201; (f) an oligonucleotide or peptide nucleic acid that hybridizes under high stringency conditions to a nucleic acid molecule including SEQ ID NO:3 having a G at position 201 but not to a nucleic acid molecule including SEQ ID NO:3 having an A at position 201; (g) an oligonucleotide or peptide nucleic acid that hybridizes under high stringency conditions to a nucleic acid molecule including SEQ ID NO:4 having a G at position 201 but not to a nucleic acid molecule including SEQ ID NO:4 having an A at position 201; (h) an oligonucleotide or peptide nucleic acid that hybridizes under high stringency conditions to a nucleic acid molecule including SEQ ID NO:4 having an A at position 201 but not to a nucleic acid molecule including SEQ ID NO:4 having a G at position 201; (i) an oligonucleotide or peptide nucleic acid that hybridizes under high stringency conditions to a nucleic acid molecule including SEQ ID NO:5 having an A at position 201 but not to a nucleic acid molecule including SEQ ID NO:5 having a C at position 201; (j) an oligonucleotide or peptide nucleic acid that hybridizes under high stringency conditions to a nucleic acid molecule including SEQ ID NO:5 having a C at position 201 but not to a nucleic acid molecule including SEQ ID NO:5 having an A at position 201; (k) an oligonucleotide or peptide nucleic acid that hybridizes under high stringency conditions to a nucleic acid molecule including SEQ ID NO:6 having an T at position 201 but not to a nucleic acid molecule including SEQ ID NO:6 having a C at position 201; (l) an oligonucleotide or peptide nucleic acid that hybridizes under high stringency conditions to a nucleic acid molecule including SEQ ID NO:6 having a C at position 201 but not to a nucleic acid molecule including SEQ ID NO:6 having an T at position 201; (m) an oligonucleotide or peptide nucleic acid that hybridizes under high stringency conditions to a nucleic acid molecule including SEQ ID NO:7 having an A at position 201 but not to a nucleic acid molecule including SEQ ID NO:7 having a C at position 201; (n) an oligonucleotide or peptide nucleic acid that hybridizes under high stringency conditions to a nucleic acid molecule including SEQ ID NO:7 having a C at position 201 but not to a nucleic acid molecule including SEQ ID NO:7 having an A at position 201; (o) an oligonucleotide or peptide nucleic acid that hybridizes under high stringency conditions to a nucleic acid molecule including SEQ ID NO:8 having a T at position 201 but not to a nucleic acid molecule including SEQ ID NO:8 having a C at position 201; (p) an oligonucleotide or peptide nucleic acid that hybridizes under high stringency conditions to a nucleic acid molecule including SEQ ID NO:8 having a C at position 201 but not to a nucleic acid molecule including SEQ ID NO:8 having a T at position 201; (q) an oligonucleotide or peptide nucleic acid that hybridizes under high stringency conditions to a nucleic acid molecule including SEQ ID NO:9 having a C at position 201 but not to a nucleic acid molecule including SEQ ID NO:9 having a T at position 201; (r) an oligonucleotide or peptide nucleic acid that hybridizes under high stringency conditions to a nucleic acid molecule including SEQ ID NO:9 having a T at position 201 but not to a nucleic acid molecule including SEQ ID NO:9 having a C at position 201; (s) an oligonucleotide or peptide nucleic acid that hybridizes under high stringency conditions to a nucleic acid molecule including SEQ ID NO:10 having a T at position 201 but not to a nucleic acid molecule including SEQ ID NO:10 having a C at position 201; (t) an oligonucleotide or peptide nucleic acid that hybridizes under high stringency conditions to a nucleic acid molecule including SEQ ID NO:10 having a C at position 201 but not to a nucleic acid molecule including SEQ ID NO:10 having a T at position 201; (u) an oligonucleotide or peptide nucleic acid capable of hybridizing under high stringency conditions to a nucleic acid molecule including a first allele for a given polymorphism selected from the polymorphisms listed in TABLE 1D but not capable of hybridizing under high stringency conditions to a nucleic acid molecule including a second allele for the given polymorphism selected from the polymorphisms listed in TABLE 1D; and (v) an oligonucleotide or peptide nucleic acid capable of hybridizing under high stringency conditions to a nucleic acid molecule including the second allele for a given polymorphism selected from the polymorphisms listed in TABLE 1D but not capable of hybridizing under high stringency conditions to a nucleic acid molecule including the first allele for the given polymorphism selected from the polymorphisms listed in TABLE 1D.

In accordance with another aspect of the invention, there is provided an array of oligonucleotides or peptide nucleic acids attached to a solid support, the array including two or more of the oligonucleotides or peptide nucleic acids as set out herein.

In accordance with another aspect of the invention, there is provided a composition including an addressable collection of two or more oligonucleotides or peptide nucleic acids, the two or more oligonucleotides or peptide nucleic acids selected from the oligonucleotides or peptide nucleic acids as set out herein.

In accordance with another aspect of the invention, there is provided a composition including an addressable collection of two or more oligonucleotides or peptide nucleic acids, the two or more oligonucleotides or peptide nucleic acids consisting essentially of two or more nucleic acid molecules set out in SEQ ID NO:1-264 or compliments, fragments, variants, or analogs thereof.

In accordance with another aspect of the invention, there is provided an composition including an addressable collection of two or more oligonucleotides or peptide nucleic acids, the two or more oligonucleotides or peptide nucleic acids consisting essentially of two or more nucleic acid molecules set out in TABLES 1C and 1D or compliments, fragments, variants, or analogs thereof. The oligonucleotides or peptide nucleic acids described herein may further include one or more of the following: a detectable label; a quencher; a mobility modifier; a contiguous non-target sequence situated 5′ or 3′ to the target sequence or 5′ and 3′ to the target sequence.

In accordance with another aspect of the invention, there is provided a computer readable medium including a plurality of digitally encoded genotype correlations selected from the vasopressin pathway associated gene SNP correlations in TABLE 1E, wherein each correlation of the plurality has a value representing an ability to recover from an inflammatory condition and a value representing an indication of responsiveness to treatment with a vasopressin receptor agonist.

The oligonucleotides or peptide nucleic acids may further include one or more of the following: a detectable label; a quencher; a mobility modifier; a contiguous non-target sequence situated 5′ or 3′ to the target sequence or 5′ and 3′ to the target sequence. The oligonucleotides or peptide nucleic acids may alternatively be of about 10 to about 400 nucleotides, about 15 to about 300 nucleotides. The oligonucleotides or peptide nucleic acids may alternatively be of about 20 to about 200 nucleotides, about 25 to about 100 nucleotides. The oligonucleotides or peptide nucleic acids may alternatively be of about 20 to about 80 nucleotides, about 25 to about 50 nucleotides. The genotype may be determined using a nucleic acid sample from the subject. Genotype may be determined using one or more of the following techniques: restriction fragment length analysis; sequencing; micro-sequencing assay; hybridization; invader assay; gene chip hybridization assays; oligonucleotide ligation assay; ligation rolling circle amplification; 5′ nuclease assay; polymerase proofreading methods; allele specific PCR; matrix assisted laser desorption ionization time of flight (MALDI-TOF) mass spectroscopy; ligase chain reaction assay; enzyme-amplified electronic transduction; single base pair extension assay; and reading sequence data. A determination of whether a site is in linkage disequilibrium (LD) with another site may be determined based on an absolute r² value or D′ value. When evaluating loci for LD those sites within a given population having a high degree of linkage disequilibrium (for example an absolute value for D′ of ≧0.5 or r²≧0.5) are potentially useful in predicting the identity of an allele of interest (for example associated with the condition of interest). A high degree of linkage disequilibrium may be represented by an absolute value for D′ of ≧0.6 or r²≧0.6. Alternatively, a higher degree of linkage disequilibrium may be represented by an absolute value for D′ of ≧0.7 or r²≧0.7 or by an absolute value for D′ of ≧0.8 or r²≧0.8. Additionally, a high degree of linkage disequilibrium may be represented by an absolute value for D′ of ≧0.85 or r²≧0.85 or by an absolute value for D′ of ≧0.9 or r²≧0.9. Two or more oligonucleotides or peptide nucleic acids may include 3 or more; 4 or more; 5 or more; 6 or more; 7 or more; 8 or more; 9 or more; 10 or more; 11 or more; 12 or more; 13 or more; 14 or more; 15 or more; 16 or more; 17 or more; 18 or more; 19 or more; or 20 or more.

Sequence variations may be assigned to a gene if mapped within 2 kb or more of an mRNA sequence feature. In particular, such a sequence may extend many kilobases (kb) from a vasopressin pathway gene and into neighbouring genes, where the LD within a region is strong.

### BRIEF DESCRIPTION OF THE DRAWINGS

FIG. 1 shows a Kaplan-Meier curve for a cohort of Caucasian Subjects with systematic inflammatory response syndrome by genotype of Leucyl aminopeptidase (LNPEP) rs18059 (CC=dashed CT/TT=solid).

FIG. 2 shows Kaplan-Meier survival curves for a cohort of Caucasian Subjects with systematic inflammatory response syndrome by genotype of Arginine Vasopressin (AVP) rs1410713 (AA=dashed CC/AC=solid).

FIG. 3 shows Kaplan-Meier survival curves for a cohort of Caucasian Subjects with sepsis by genotype of Arginine Vasopressin (AVP) rs1410713 (AA=dashed CC/AC=solid).

FIG. 4 shows Kaplan-Meier survival curves for a cohort of Caucasian Subjects with septic shock by genotype of Arginine Vasopressin (AVP) rs1410713 (AA=dashed CC/AC=solid).

FIG. 5 shows Kaplan-Meier survival curves for a cohort of Caucasian Subjects with systematic inflammatory response syndrome by genotype of Arginine Vasopressin (AVP) rs857242 (AC/AA=solid vs. CC=dashed).

FIG. 6 shows Kaplan-Meier survival curves for a cohort of Caucasian Subjects with sepsis by genotype of Arginine Vasopressin (AVP) rs857242 (AC/AA=solid vs. CC=dashed).

FIG. 7 shows Kaplan-Meier survival curves for a cohort of Caucasian Subjects with septic shock by genotype of Arginine Vasopressin (AVP) rs857242 (AC/AA=solid vs. CC=dashed).

FIG. 8 shows Kaplan-Meier survival curves for a cohort of Caucasian Subjects with systematic inflammatory response syndrome by genotype of arginine vasopressin receptor (AVPR1A) rs3803107 (CC/CT=solid vs. TT=dashed).

FIG. 9 shows a Kaplan Meier survival curve over 28 days for a cohort of Asian Subjects with systematic inflammatory response syndrome by allele of arginine vasopressin receptor (AVPR1A) rs3803107 (C=solid vs. T=dashed).

FIG. 10 shows a Kaplan Meier survival curve over 28 days for a cohort of Asian Subjects with systematic inflammatory response syndrome by allele of arginine vasopressin receptor (AVPR1A) rs10877970 (T=dashed vs. C=solid).

### DETAILED DESCRIPTION OF THE INVENTION

### 1. Definitions

In the description that follows, a number of terms are used extensively, the following definitions are provided to facilitate understanding of the invention.

“Vasopressin Receptor Agonist” as used herein includes any vasopressin molecule, vasopressin derivative, vasopressin variant, vasopressin analogue, non-peptidyl analogues and any prodrug thereof, metabolite thereof, isomer thereof, combination of isomers thereof, or pharmaceutical composition of any of the preceding. Such agonists may be capable of binding to or interacting with a vasopressin receptor and initiating one or more of the types of responses typically produced by the binding of an endogenous vasopressin molecule to a vasopressin receptor (for example, AVPR1A, AVPR1B, AVPR2 and OXTR). Such activity may be present at the time of or following, administration to a subject. Vasopressin receptor agonists may be used alone or in combination with other vasopressin receptor agonists or other medications. Vasopressin receptor agonists may be synthesized or purified. Examples of vasopressin receptor agonists capable of increasing blood pressure, include, but are not limited to, arginine vasopressin (AVP), lysine vasopressin (LVP), triglycil-lysine vasopressin (also known as Terlipressin or Glycopressin), Octapressin, Ornipressin, Desmopressin, Desmopressin acetate, Lypressin, Felypressin, and Argipressin. Vasopressin analogues may be 1-3 amino acids such as Ala-AVP, Ser-Ala-AVP, Thr-Ser-Ala-AVP (KALISZAN R. et al. Pharmacol Res Commun (1988) 20(5):377-381) or 3-beta-(2-thienyl)-L-alanine)-8-lysine-vasopressin and other similar analogues (Smith C W. Acta Pharmacol Toxicol (Copenhag) (1978) 43(3): 190-195). Examples of derivatives, variants, analogues or compositions etc. may found in US patent applications: 20050075328; 20040229798; 20030134845; 20030021792; 20030018024; 20030008863; 20030004159; 20020198196; 20020198191; 20020049194; 20050075328; 20040229798; 20030018024; and 20020198191 and issued U.S. Pat. Nos. 6,903,091; 6,831,079; 6,642,223; 6,620,807; 6,511,974; 6,344,451; 6,335,327; 6,297,234; 6,268,360; 6,235,900; 6,204,260; 6,194,407; 6,096,736; 6,096,735; 6,090,803; 4,908,475; 4,810,778; 4,760,052; 4,711,877; 6,903,091; 6,620,807; 6,344,451; 6,297,234; and 6,268,360.

“Vasopressin” as used herein includes: Antidiuretic hormone; Argiprestocin; Arginine Vasopressin; Arginine oxytocin; Pitressin tannate; Arginine vasotocin; Vasotocin; Vasopressin, isoleucyl; 3-Isoleucyl vasopressin; 1-[[19-amino-13-butan-2-yl-10-(2-carbamoylethyl)-7-(carbamoyl methyl)-16-[(4-hydroxyphenyl)methyl]-6,9,12,15,18-pentaoxo-1,2-dithia-5,8,11,14,17-pentazacycloicos-4-yl]carbonyl]-N-[1-(carbamoylmethylcarbamoyl)-4-guanidino-butyl]-pyrrolidine-2-carboxamide (IUPAC name). Vasopressin is a nine amino acid peptide (Cys-Tyr-Ile-Gln-Asn-Cys-Pro-Arg-Gly, cyclic 1-6 disulfide) secreted from the posterior pituitary and binds to receptors in blood vessels, the brain and distal or collecting tubules of the kidney to promote vasoconstriction or reabsorption of water back into the circulation. Vasopressin receptor targets, include AVPR1A, AVPR1B, AVPR2 and OXTR. Vasopressin, for example, is sold as PRESSYN AR™ by Ferring Inc., and also sold in various formulations as VASOPRESSIN by Ferring Inc., Sandoz Canada Inc. and Pharmaceutical Partners of Canada Inc. Similarly, PITRESSIN™ is sold by Warner-Lambert Company, Parke-Davis Division, as a synthetic injectable vasopressin (8-Arginine vasopressin). It is substantially free from the oxytocic principle and is standardized to contain 20 pressor units/mL. The solution contains 0.5% Chlorobutanol (chloroform derivative) as a preservative. Also, DIAPID™ is sold as a nasal spray by Sandoz Inc. The current published indications for vasopressin (from the label of Ferring's PRESSYN AR™) are “Vasopressin is intended for use in the prevention of treatment of post-operative abdominal distension, dispelling of gas shadows in abdominal roentgenography and symptomatic control of diabetes insipidus.”

“Genetic material” includes any nucleic acid and can be a deoxyribonucleotide or ribonucleotide polymer in either single or double-stranded form.

A “purine” is a heterocyclic organic compound containing fused pyrimidine and imidazole rings, and acts as the parent compound for purine bases, adenine (A) and guanine (G). A “Nucleotide” is generally a purine (R) or pyrimidine (Y) base covalently linked to a pentose, usually ribose or deoxyribose, where the sugar carries one or more phosphate groups. Nucleic acids are generally a polymer of nucleotides joined by 3′-5′ phosphodiester linkages. As used herein “purine” is used to refer to the purine bases, A and G, and more broadly to include the nucleotide monomers, deoxyadenosine-5′-phosphate and deoxyguanosine-5′-phosphate, as components of a polynucleotide chain.

A “pyrimidine” is a single-ringed, organic base that forms nucleotide bases, cytosine (C), thymine (T) and uracil (U). As used herein “pyrimidine” is used to refer to the pyrimidine bases, C, T and U, and more broadly to include the pyrimidine nucleotide monomers that along with purine nucleotides are the components of a polynucleotide chain.

A nucleotide represented by the symbol M may be either an A or C, a nucleotide represented by the symbol W may be either an T/U or A, a nucleotide represented by the symbol Y may be either an C or T/U, a nucleotide represented by the symbol S may be either an G or C, while a nucleotide represented by the symbol R may be either an G or A, and a nucleotide represented by the symbol K may be either an G or T/U. Similarly, a nucleotide represented by the symbol V may be either A or G or C, while a nucleotide represented by the symbol D may be either A or G or T, while a nucleotide represented by the symbol B may be either G or C or T, and a nucleotide represented by the symbol H may be either A or C or T.

A “polymorphic site” or “polymorphism site” or “polymorphism” or “single nucleotide polymorphism site” (SNP site) or single nucleotide polymorphism” (SNP) as used herein is the locus or position with in a given sequence at which divergence occurs. A “polymorphism” is the occurrence of two or more forms of a gene or position within a gene (allele), in a population, in such frequencies that the presence of the rarest of the forms cannot be explained by mutation alone. The implication is that polymorphic alleles confer some selective advantage on the host. Preferred polymorphic sites have at least two alleles, each occurring at frequency of greater than 1%, and more preferably greater than 10% or 20% of a selected population. Polymorphic sites may be at known positions within a nucleic acid sequence or may be determined to exist using the methods described herein. Polymorphisms may occur in both the coding regions and the noncoding regions (for example, promoters, introns or untranslated regions) of genes. Polymorphisms may occur at a single nucleotide site (SNPs) or may involve an insertion or deletion as described herein.

A “risk genotype” as used herein refers to an allelic variant (genotype) at one or more polymorphic sites within the vasopressin pathway gene (i.e. AVP, AVPR1A and LNPEP) sequences described herein as being indicative of a decreased likelihood of recovery from an inflammatory condition or an increased risk of having a poor outcome. The risk genotype may be determined for either the haploid genotype or diploid genotype, provided that at least one copy of a risk allele is present.

Risk genotype may be an indication of an increased risk of not recovering from an inflammatory condition. Subjects having one copy (heterozygotes) or two copies (homozygotes) of the risk allele (for example rs18059 CT, rs18059 TT) are considered to have the “risk genotype” even though the degree to which the subjects risk of not recovering from an inflammatory condition may increase, depending on whether the subject is a homozygote rather than a heterozygote. Such “risk alleles” or “risk genotypes” may be selected from the following: rs18059CT; rs18059TT; rs27711GA; rs27711GG; rs38041GA; rs38041GG; rs10051637GA; rs10051637GG; rs1410713AA; rs857240CC; rs857242CC; rs10877970TT; rs3803107TT; and rs1495027CC; or a polymorphic site in linkage disequilibrium thereto.

A “decreased risk genotype” as used herein refers to an allelic variant (genotype) at one or more polymorphic sites within the vasopressin pathway gene (i.e. AVP, AVPR1A and LNPEP) sequences described herein as being indicative of an increased likelihood of recovery from an inflammatory condition or a decreased risk of having a poor outcome. The decreased risk genotype may be determined for either the haploid genotype or diploid genotype, provided that at least one copy of a risk allele is present. Decreased risk genotype may be an indication of an increased likelihood of recovering from an inflammatory condition. Subjects having one copy (heterozygotes) or two copies (homozygotes) of the decreased risk allele (for example rs1410713 CC rs1410713 AC) are considered to have the “decreased risk genotype” even though the degree to which the subjects risk of not recovering from an inflammatory condition may increase, depending on whether the subject is a homozygote rather than a heterozygote. Such “decreased risk alleles” or “decreased risk genotypes” or “reduced risk genotypes” may be selected from the following: rs18059CC; rs27711AA; rs38041AA; rs10051637AA; rs1410713CC; rs1410713AC; rs857240TT; rs857240CT; rs857242AA; rs857242AC; rs10877970TT; rs10877970CT; rs3803107CC; rs3803107CT; rs1495027CC and rs1495027CT; or a polymorphic site in linkage disequilibrium thereto.

An “improved response genotype” (IRG) or improved response polymorphic variant (IRP) as used herein refers to an allelic variant or genotype at one or more polymorphic sites within the vasopressin pathway associated polymorphisms selected from arginine vasopressin (AVP), arginine vasopressin receptor 1A (AVPR1A) leucyl/cystinyl aminopeptidase (LNPEP) or leukocyte-derived aminopeptidase (LRAP) as described herein as being predictive of a subject's improved survival in response to vasopressin receptor agonist treatment (for example rs18059TT, rs27711GG, rs10051637AA, rs1410713AA, rs857240CC, rs857242CC or rs1495027CC), or a polymorphic site in linkage disequilibrium thereto.

An “adverse response genotype” (ARG) or adverse response polymorphic variant as used herein refers to an allelic variant or genotype at one or more polymorphic sites within the vasopressin pathway associated polymorphisms selected from arginine vasopressin (AVP), arginine vasopressin receptor 1A (AVPR1A), leucyl/cystinyl aminopeptidase (LNPEP) or leukocyte-derived aminopeptidase (LRAP) as described herein as being predictive of a subject's decreased survival in response to vasopressin receptor agonist treatment (for example rs18059CC, rs27711AA, rs10051637GG, rs1410713CC, rs857240CT, rs857242AC or rs1495027TT), or a polymorphic site in linkage disequilibrium thereto. Subjects having a ARG are preferably selected for treatments not involving vasopressin receptor agonist administration.

A “clade” is a group of haplotypes that are closely related phylogenetically. For example, if haplotypes are displayed on a phylogenetic (evolutionary) tree a clade includes all haplotypes contained within the same branch.

The pattern of a set of markers along a chromosome is referred to as a “Haplotype”. Accordingly, groups of alleles on the same small chromosomal segment tend to be transmitted together. Haplotypes along a given segment of a chromosome are generally transmitted to progeny together unless there has been a recombination event. Absence of a recombination event, haplotypes can be treated as alleles at a single highly polymorphic locus for mapping.

As used herein “haplotype” is a set of alleles of closely linked loci on a chromosome that tend to be inherited together. Such allele sets occur in patterns, which are called haplotypes. Accordingly, a specific SNP or other polymorphism allele at one SNP site is often associated with a specific SNP or other polymorphism allele at a nearby second SNP site or other polymorphism site. When this occurs, the two SNPs or other polymorphisms are said to be in LD because the two SNPs or other polymorphisms are not just randomly associated (i.e. in linkage equilibrium).

In general, the detection of nucleic acids in a sample depends on the technique of specific nucleic acid hybridization in which the oligonucleotide is annealed under conditions of “high stringency” to nucleic acids in the sample, and the successfully annealed oligonucleotides are subsequently detected (see for example Spiegelman, S., Scientific American, Vol. 210, p. 48 (1964)). Hybridization under high stringency conditions primarily depends on the method used for hybridization, the oligonucleotide length, base composition and position of mismatches (if any). High-stringency hybridization is relied upon for the success of numerous techniques routinely performed by molecular biologists, such as high-stringency PCR, DNA sequencing, single strand conformational polymorphism analysis, and in situ hybridization. In contrast to Northern and Southern hybridizations, these aforementioned techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998.

“Oligonucleotides” as used herein are variable length nucleic acids, which may be useful as probes, primers and in the manufacture of microarrays (arrays) for the detection and/or amplification of specific nucleic acids. Such DNA or RNA strands may be synthesized by the sequential addition (5′-3′ or 3′-5′) of activated monomers to a growing chain, which may be linked to an insoluble support. Numerous methods are known in the art for synthesizing oligonucleotides for subsequent individual use or as a part of the insoluble support, for example in arrays (BERNHELD M R. and ROTTMAN F M. J. Biol. Chem. (1967) 242(18):4134-43; SULSTON J. et al. PNAS (1968) 60(2):409-415; GILLAM S. et al. Nucleic Acid Res. (1975) 2(5):613-624; BONORA G M. et al. Nucleic Acid Res. (1990) 18(11):3155-9; LASHKARI D A. et al. Proc Nat Acad Sci (1995) 92(17):7912-5; MCGALL G. et al. PNAS (1996) 93(24): 13555-60; ALBERT T J. et al. Nucleic Acid Res. (2003) 31(7):e35; GAO X. et al. Biopolymers (2004) 73(5):579-96; and MOORCROFT M J. et al. Nucleic Acid Res. (2005) 33(8):e75). In general, oligonucleotides are synthesized through the stepwise addition of activated and protected monomers under a variety of conditions depending on the method being used. Subsequently, specific protecting groups may be removed to allow for further elongation and subsequently and once synthesis is complete all the protecting groups may be removed and the oligonucleotides removed from their solid supports for purification of the complete chains if so desired.

“Peptide nucleic acids” (PNA) as used herein refer to modified nucleic acids in which the sugar phosphate skeleton of a nucleic acid has been converted to an N-(2-aminoethyl)-glycine skeleton. Although the sugar-phosphate skeletons of DNA/RNA are subjected to a negative charge under neutral conditions resulting in electrostatic repulsion between complementary chains, the backbone structure of PNA does not inherently have a charge. Therefore, there is no electrostatic repulsion.

Consequently, PNA has a higher ability to form double strands as compared with conventional nucleic acids, and has a high ability to recognize base sequences. Furthermore, PNAs are generally more robust than nucleic acids. PNAs may also be used in arrays and in other hybridization or other reactions as described above and herein for oligonucleotides.

An “addressable collection” as used herein is a combination of nucleic acid molecules or peptide nucleic acids capable of being detected by, for example, the use of hybridization techniques or by any other means of detection known to those of ordinary skill in the art. A DNA microarray would be considered an example of an “addressable collection”.

In general the term “linkage”, as used in population genetics, refers to the co-inheritance of two or more nonallelic genes or sequences due to the close proximity of the loci on the same chromosome, whereby after meiosis they remain associated more often than the 50% expected for unlinked genes. However, during meiosis, a physical crossing between individual chromatids may result in recombination. “Recombination” generally occurs between large segments of DNA, whereby contiguous stretches of DNA and genes are likely to be moved together in the recombination event (crossover). Conversely, regions of the DNA that are far apart on a given chromosome are more likely to become separated during the process of crossing-over than regions of the DNA that are close together. Polymorphic molecular markers, like SNPs, are often useful in tracking meiotic recombination events as positional markers on chromosomes.

Furthermore, the preferential occurrence of a disease gene in association with specific alleles of linked markers, such as SNPs or other polymorphisms, is called “Linkage Disequilibrium” (LD). This sort of disequilibrium generally implies that most of the disease chromosomes carry the same mutation and the markers being tested are relatively close to the disease gene(s).

For example, in SNP-based association analysis and LD mapping, SNPs can be useful in association studies for identifying polymorphisms, associated with a pathological condition, such as sepsis. Unlike linkage studies, association studies may be conducted within the general population and are not limited to studies performed on related individuals in affected families. In a SNP association study the frequency of a given allele (i.e. SNP allele) is determined in numerous subjects having the condition of interest and in an appropriate control group. Significant associations between particular SNPs or SNP haplotypes and phenotypic characteristics may then be determined by numerous statistical methods known in the art.

Association analysis can either be direct or LD based. In direct association analysis, potentially causative SNPs may be tested as candidates for the pathogenic sequence. In LD based SNP association analysis, SNPs may be chosen at random over a large genomic region or even genome wide, to be tested for SNPs in LD with a pathogenic sequence or pathogenic SNP. Alternatively, candidate sequences associated with a condition of interest may be targeted for SNP identification and association analysis. Such candidate sequences usually are implicated in the pathogenesis of the condition of interest. In identifying SNPs associated with inflammatory conditions, candidate sequences may be selected from those already implicated in the pathway of the condition or disease of interest. Once identified, SNPs found in or associated with such sequences, may then be tested for statistical association with an individual's prognosis or susceptibility to the condition.

For an LD based association analysis, high density SNP maps are useful in positioning random SNPs relative to an unknown pathogenic locus. Furthermore, SNPs tend to occur with great frequency and are often spaced uniformly throughout the genome. Accordingly, SNPs as compared with other types of polymorphisms are more likely to be found in close proximity to a genetic locus of interest. SNPs are also mutationally more stable than variable number tandem repeats (VNTRs) and short tandem repeats (STRs).

In population genetics linkage disequilibrium refers to the “preferential association of a particular allele, for example, a mutant allele for a disease with a specific allele at a nearby locus more frequently than expected by chance” and implies that alleles at separate loci are inherited as a single unit (Gelehrter, T. D., Collins, F. S. (1990). Principles of Medical Genetics. Baltimore: Williams & Wilkens). Accordingly, the alleles at these loci and the haplotypes constructed from their various combinations serve as useful markers of phenotypic variation due to their ability to mark clinically relevant variability at a particular position, such as position 201 of SEQ ID NO:1 (see Akey, J. et al. Eur J Hum Genet (2001) 9:291-300; and Zhang, K. et al. (2002). Am J Hum Genet. 71:1386-1394). This viewpoint is further substantiated by Khoury et al. ((1993). Fundamentals of Genetic Epidemiology. New York: Oxford University Press at p. 160) who state, “[w]henever the marker allele is closely linked to the true susceptibility allele and is in [linkage] disequilibrium with it, one can consider that the marker allele can serve as a proxy for the underlying susceptibility allele.”

As used herein “linkage disequilibrium” (LD) is the occurrence in a population of certain combinations of linked alleles in greater proportion than expected from the allele frequencies at the loci. For example, the preferential occurrence of a disease gene in association with specific alleles of linked markers, such as SNPs, or between specific alleles of linked markers, are considered to be in LD. This sort of disequilibrium generally implies that most of the disease chromosomes carry the same mutation and that the markers being tested are relatively close to the disease gene(s). Accordingly, if the genotype of a first locus is in LD with a second locus (or third locus etc.), the determination of the allele at only one locus would necessarily provide the identity of the allele at the other locus. When evaluating loci for LD those sites within a given population having a high degree of linkage disequilibrium (i.e. an absolute value for r²≧0.5) are potentially useful in predicting the identity of an allele of interest (i.e. associated with the condition of interest). A high degree of linkage disequilibrium may be represented by an absolute value for r²≧0.6. Alternatively, a high degree of linkage disequilibrium may be represented by an absolute value for r²≧0.7 or by an absolute value for r²≧0.8. Additionally, a high degree of linkage disequilibrium may be represented by an absolute value for r²≧0.85 or by an absolute value for r²≧0.9. Accordingly, two SNPs that have a high degree of LD may be equally useful in determining the identity of the allele of interest or disease allele. Therefore, we may assume that knowing the identity of the allele at one SNP may be representative of the allele identity at another SNP in LD. Accordingly, the determination of the genotype of a single locus can provide the identity of the genotype of any locus in LD therewith and the higher the degree of linkage disequilibrium the more likely that two SNPs may be used interchangeably. For example, in the population from which the tagged SNPs were identified from the SNP identified by rs18059 is in “linkage disequilibrium” with the SNP identified by rs2762, whereby when the genotype of rs18059 is T the genotype of rs2762 is G. Similarly, when the genotype of rs18059 is C the genotype of rs2762 is A. Accordingly, the determination of the genotype at rs18059 will provide the identity of the genotype at rs2762 or any other locus in “linkage disequilibrium” therewith. Particularly, where such a locus is has a high degree of linkage disequilibrium thereto.

LD is useful for genotype-phenotype association studies. For example, if a specific allele at one SNP site (e.g. “A”) is the cause of a specific clinical outcome (e.g. call this clinical outcome “B”) in a genetic association study then, by mathematical inference, any SNP (e.g. “C”) which is in significant LD with the first SNP, will show some degree of association with the clinical outcome. That is, if A is associated (˜) with B, i.e. A˜B and C˜A then it follows that C˜B. Of course, the SNP that will be most closely associated with the specific clinical outcome, B, is the causal SNP—the genetic variation that is mechanistically responsible for the clinical outcome. Thus, the degree of association between any SNP, C, and clinical outcome will depend on LD between A and C.

Until the mechanism underlying the genetic contribution to a specific clinical outcome is fully understood, LD helps identify potential candidate causal SNPs and also helps identify a range of SNPs that may be clinically useful for prognosis of clinical outcome or of treatment effect. If one SNP within a gene is found to be associated with a specific clinical outcome, then other SNPs in LD will also have some degree of association and therefore some degree of prognostic usefulness.

By way of prophetic example, if multiple polymorphisms were tested for individual association with an improved response to vasopressin receptor agonist administration in our SIRS/sepsis/septic shock cohort of ICU subjects, wherein the multiple polymorphisms had a range of LD with LNPEP rs18059 and it was assumed that rs18059 was the causal polymorphism, and we were to order the polymorphisms by the degree of LD with rs18059, we would expect to find that polymorphisms with high degrees of LD with rs18059 would also have a high degree of association with this specific clinical outcome. As LD decreased, we would expect the degree of association of the polymorphism with an improved response vasopressin receptor agonist administration to also decrease. It follows that any polymorphism, whether already discovered or as yet undiscovered, that is in LD with one of the improved response genotypes described herein will likely be a predictor of the same clinical outcomes that rs18059 is a predictor of. The similarity in prediction between this known or unknown polymorphism and rs18059 would depend on the degree of LD between such a polymorphism and rs18059.

Numerous sites have been identified as polymorphic sites in the vasopressin pathway associated genes (see TABLE 1A). Furthermore, the polymorphisms in TABLE 1A are linked to (in LD with) numerous polymorphism as set out in TABLE 1B below and may also therefore be indicative of subject prognosis.

TABLE 1A
Polymorphisms in the vasopressin pathway associated genes genotyped in a cohort of
critically ill Subjects with severe sepsis. Minor Allele Frequencies (MAFs) for Caucasians were
taken from Hapmap.org (Thorisson GA. et al. The International HapMap Project Website.
Genome Research (2005)15: 1591-1593).

| | | | March 2006 | | |
|---|---|---|---|---|---|
| | | | Chromosomal | | Minor |
| Polymorphism Name | Official Gene | | position | Minor | Allele |
| (Alleles) | Name | rs# | (Build 36) | allele | Frequency |
| LNPEP rs18059 (C/T) | leucyl/cystinyl | rs18059 | 96377824 | T | 0.39 |
| | aminopeptidase | | | | |
| | (LNPEP) | | | | |
| LNPEP rs27711 (G/A) | leucyl/cystinyl | rs27711 | 96371495 | A | 0.49 |
| | aminopeptidase | | | | |
| | (LNPEP) | | | | |
| LNPEP rs38041 (A/G) | leucyl/cystinyl | rs38041 | 96356058 | G | 0.48 |
| | aminopeptidase | | | | |
| | (LNPEP) | | | | |
| LNPEP rs10051637 (A/G) | leucyl/cystinyl | rs10051637 | 96305246 | G | 0.49 |
| | aminopeptidase | | | | |
| | (LNPEP) | | | | |
| AVP rs1410713 (A/C) | arginine vasopressin | rs1410713 | 3008350 | C | 0.44 |
| | (AVP) | | | | |
| AVP rs857240 (C/T) | arginine vasopressin | rs857240 | 3023629 | T | 0.09 |
| | (AVP) | | | | |
| AVP rs857242 (C/A) | arginine vasopressin | rs857242 | 3029101 | A | 0.1 |
| | (AVP) | | | | |
| AVPR1A rs10877970 (T/C) | arginine vasopressin | rs10877970 | 61837421 | C | 0.09 |
| | receptor | | | | |
| | 1A(AVPR1A) | | | | |
| AVPR1A rs3803107 (C/T) | arginine vasopressin | rs3803107 | 61827101 | T | 0.13 |
| | receptor | | | | |
| | 1A(AVPR1A) | | | | |
| AVPR1A rs1495027 (C/T) | arginine vasopressin | rs1495027 | 61890334 | T | 0.42 |
| | receptor | | | | |
| | 1A(AVPR1A) | | | | |

TABLE 1B
Polymorphisms in linkage disequilibrium with those listed in TABLE 1A above, as
identified using the Haploview program (BARRETT JC. et al. Bioinformatics (2005) 21(2): 263-5
(http://www.broad.mit.edu/mpg/haploview/)) and the LD function in the Genetics Package in R (R
Core Development Group, 2005-R Development Core Team (www.R-project.org). Linkage
Disequilibrium between markers was defined using r² whereby all SNPs available on Hapmap.org
(phase II) (cohort H), all SNPs genotyped internally using the Illumina Goldengate assay (cohort I)
and all SNPs sequenced using the Sequenom Iplex Platform (cohort S) in our genes of interest
were included. A minimum r² of 0.5 was used as the cutoff to identify LD SNPs. The genes are
identified, along with the alleles, rs designation and the chromosomal position (March 2006 Build
36). An LD allele was only predicted for those cohorts that had sufficient power and NA
designations indicate that the sample sizes were insufficient to make an allele designation with
confidence at the time of filing. However, the assignment of allele designations for NA designated
LD alleles is a routine procedure.

| | Tag | | | | | | RSIDs of |
|---|---|---|---|---|---|---|---|
| | SNP | Tag | Polymorphism | | | Polymorphism | Polymorphism |
| Gene | (IRP) | Polymorphisms | RSID | Cohort | LD Allele | in LD | in LD |
| LNPEP | TC | 96377824 | rs18059 | H/I | C | 96346251 | rs10044354 |
| | (T) | | | | | | |
| | | | | H/I | A | 96305246 | rs10051637 |
| | | | | H | T | 96323283 | rs10058476 |
| | | | | I | T | 96345363 | rs10476696 |
| | | | | S | NA | 96238651 | rs1230360 |
| | | | | S | NA | 96240263 | rs1230363 |
| | | | | S | NA | 96240337 | rs1230364 |
| | | | | S | NA | 96240415 | rs1230365 |
| | | | | H | G | 96278559 | rs1363907 |
| | | | | H/I | A | 96319572 | rs1363974 |
| | | | | H/I | T | 96324514 | rs1423357 |
| | | | | H | G | 96310648 | rs1477364 |
| | | | | H | A | 96339986 | rs1544777 |
| | | | | H | A | 96259206 | rs2113189 |
| | | | | H/I | G | 96343901 | rs2161548 |
| | | | | H/I | C | 96319685 | rs2351010 |
| | | | | H | A | 96396635 | rs248215 |
| | | | | I | A | 96298789 | rs2548225 |
| | | | | H | G | 96265683 | rs2548530 |
| | | | | H | A | 96264334 | rs2548532 |
| | | | | H | C | 96257128 | rs2549783 |
| | | | | H | A | 96268198 | rs2549791 |
| | | | | H | T | 96270305 | rs2549794 |
| | | | | S | NA | 96239682 | rs2617436 |
| | | | | H | T | 96293411 | rs2617447 |
| | | | | I | T | 96372034 | rs27289 |
| | | | | H/I | A | 96375844 | rs27290 |
| | | | | H | G | 96383016 | rs27294 |
| | | | | H | T | 96387382 | rs27296 |
| | | | | H | T | 96389163 | rs27300 |
| | | | | H | T | 96360314 | rs27306 |
| | | | | H | G | 96364261 | rs27307 |
| | | | | H | G | 96366372 | rs27397 |
| | | | | H/I | C | 96356722 | rs27436 |
| | | | | H | G | 96365029 | rs27613 |
| | | | | H/I | G | 96299054 | rs2762 |
| | | | | H/I | G | 96369308 | rs27659 |
| | | | | H | G | 96371495 | rs27711 |
| | | | | H | G | 96385668 | rs27747 |
| | | | | H | T | 96278345 | rs2910686 |
| | | | | I | T | 96302142 | rs2910792 |
| | | | | H | C | 96277835 | rs2927609 |
| | | | | S | NA | 96239688 | rs35199417 |
| | | | | H/I | G | 96342514 | rs3797796 |
| | | | | H | T | 96379440 | rs38030 |
| | | | | H | T | 96347643 | rs38032 |
| | | | | H/I | A | 96347892 | rs38033 |
| | | | | H/I | C | 96348175 | rs38034 |
| | | | | H | C | 96349036 | rs38035 |
| | | | | H | A | 96349259 | rs38036 |
| | | | | H | G | 96356058 | rs38041 |
| | | | | H/I | G | 96362547 | rs38043 |
| | | | | H | T | 96260289 | rs3849749 |
| | | | | H | G | 96390210 | rs39602 |
| | | | | H | A | 96318909 | rs4360063 |
| | | | | H | G | 96251952 | rs6556942 |
| | | | | I | C | 96307418 | rs6871162 |
| | | | | H | G | 96290756 | rs716848 |
| | | | | I | G | 96315986 | rs7703341 |
| | | | | H | A | 96313894 | rs7713127 |
| | | | | H | C | 96318762 | rs7716222 |
| | | | | H | G | 96312042 | rs7719705 |
| | | | | H/I | G | 96314716 | rs7731592 |
| | | | | H | G | 96315467 | rs7736466 |
| | | | | I | T | 96346342 | rs9314181 |
| LNEP | GA | 96371495 | rs27711 | S | NA | 96346167 | rs10038651 |
| | (G) | | | | | | |
| | | | | H/I | C | 96346251 | rs10044354 |
| | | | | H/I | A | 96305246 | rs10051637 |
| | | | | H | T | 96323283 | rs10058476 |
| | | | | S | NA | 96309577 | rs10061936 |
| | | | | H/I | G | 96326898 | rs10071975 |
| | | | | H/I | G | 96280573 | rs1019503 |
| | | | | S | NA | 96251278 | rs10434708 |
| | | | | I | G | 96298936 | rs1046395 |
| | | | | I | T | 96345363 | rs10476696 |
| | | | | S | NA | 96276415 | rs10537702 |
| | | | | S | NA | 96276948 | rs10546363 |
| | | | | H/I | T | 96271195 | rs1056893 |
| | | | | S | NA | 96284454 | rs10592692 |
| | | | | S | NA | 96255974 | rs10707238 |
| | | | | I | G | 96247321 | rs11135483 |
| | | | | I | G | 96247645 | rs11135484 |
| | | | | S | NA | 96312725 | rs11135485 |
| | | | | S | NA | 96357847 | rs11311774 |
| | | | | S | NA | 96370825 | rs11414909 |
| | | | | I | A | 96247097 | rs11750025 |
| | | | | H | C | 96291635 | rs1216565 |
| | | | | S | NA | 96289812 | rs1216566 |
| | | | | S | NA | 96289595 | rs1216567 |
| | | | | S | NA | 96289402 | rs1216568 |
| | | | | S | NA | 96288290 | rs1216569 |
| | | | | S | NA | 96287473 | rs1216570 |
| | | | | I | T | 96246767 | rs12189125 |
| | | | | H | G | 96237497 | rs1230358 |
| | | | | I | A | 96279965 | rs1230381 |
| | | | | H | T | 96254538 | rs12516666 |
| | | | | H | A | 96333392 | rs12716486 |
| | | | | I | C | 96247776 | rs13167902 |
| | | | | S | NA | 96304809 | rs13170029 |
| | | | | I | A | 96248383 | rs13189819 |
| | | | | S | NA | 96321566 | rs13358339 |
| | | | | H/I | G | 96278559 | rs1363907 |
| | | | | S | NA | 96278860 | rs1363908 |
| | | | | H/I | A | 96319572 | rs1363974 |
| | | | | S | NA | 96274642 | rs1363975 |
| | | | | S | NA | 96274551 | rs1363976 |
| | | | | I | A | 96274463 | rs1363977 |
| | | | | H/I | T | 96324514 | rs1423357 |
| | | | | I | A | 96299523 | rs1423566 |
| | | | | H | G | 96310648 | rs1477364 |
| | | | | H | A | 96339986 | rs1544777 |
| | | | | I | T | 96329454 | rs1559267 |
| | | | | I | G | 96249877 | rs1559354 |
| | | | | H/I | T | 96252451 | rs1559355 |
| | | | | S | NA | 96252485 | rs1559356 |
| | | | | S | NA | 96252486 | rs1559357 |
| | | | | S | NA | 96268737 | rs17087165 |
| | | | | H | T | 96377824 | rs18059 |
| | | | | S | NA | 96278754 | rs1820148 |
| | | | | S | NA | 96332914 | rs1820149 |
| | | | | H/I | G | 96262870 | rs187265 |
| | | | | H | C | 96252291 | rs1974871 |
| | | | | H/I | G | 96294618 | rs1981846 |
| | | | | S | NA | 96260377 | rs2042383 |
| | | | | H | G | 96273749 | rs2042385 |
| | | | | S | NA | 40328876 | rs210687 |
| | | | | H/I | A | 96340258 | rs2113050 |
| | | | | H | A | 96259206 | rs2113189 |
| | | | | I | A | 96262074 | rs2113190 |
| | | | | H/I | G | 96343901 | rs2161548 |
| | | | | H/I | T | 96258562 | rs2161657 |
| | | | | H/I | C | 96265401 | rs2161658 |
| | | | | H/I | A | 96255506 | rs2247650 |
| | | | | H | A | 96274871 | rs2255546 |
| | | | | H | T | 96275079 | rs2255633 |
| | | | | H | T | 96275107 | rs2255634 |
| | | | | H | G | 96275134 | rs2255637 |
| | | | | H/I | T | 96250335 | rs2278018 |
| | | | | I | A | 96251008 | rs2278019 |
| | | | | H/I | A | 96263082 | rs2287988 |
| | | | | S | NA | 96247939 | rs2303208 |
| | | | | I | T | 96247941 | rs2303209 |
| | | | | H/I | C | 96319685 | rs2351010 |
| | | | | S | NA | 96277431 | rs2351011 |
| | | | | H | A | 96396635 | rs248215 |
| | | | | H/I | C | 96260794 | rs251339 |
| | | | | S | NA | 96262168 | rs251340 |
| | | | | S | NA | 96283585 | rs251343 |
| | | | | H | G | 96284683 | rs251344 |
| | | | | I | A | 96298789 | rs2548225 |
| | | | | I | G | 96301169 | rs2548516 |
| | | | | S | NA | 96276759 | rs2548520 |
| | | | | S | NA | 96276684 | rs2548521 |
| | | | | I | T | 96276213 | rs2548522 |
| | | | | H | A | 96272696 | rs2548523 |
| | | | | H/I | A | 96272357 | rs2548524 |
| | | | | H | G | 96270341 | rs2548527 |
| | | | | H/I | G | 96265976 | rs2548529 |
| | | | | H/I | G | 96265683 | rs2548530 |
| | | | | H | A | 96264334 | rs2548532 |
| | | | | H | C | 96264157 | rs2548533 |
| | | | | H | T | 96258158 | rs2548536 |
| | | | | H/I | T | 96257898 | rs2548538 |
| | | | | H | A | 96257260 | rs2548539 |
| | | | | H | T | 96255934 | rs2548540 |
| | | | | H | T | 96255878 | rs2549781 |
| | | | | H | T | 96256756 | rs2549782 |
| | | | | H/I | C | 96257128 | rs2549783 |
| | | | | H | T | 96257276 | rs2549784 |
| | | | | S | NA | 96265593 | rs2549787 |
| | | | | S | NA | 96268026 | rs2549789 |
| | | | | H | C | 96268168 | rs2549790 |
| | | | | H/I | A | 96268198 | rs2549791 |
| | | | | H/I | T | 96270305 | rs2549794 |
| | | | | H | G | 96270394 | rs2549795 |
| | | | | H/I | T | 96271099 | rs2549796 |
| | | | | H/I | G | 96271274 | rs2549797 |
| | | | | S | NA | 96271659 | rs2549798 |
| | | | | S | NA | 96271666 | rs2549799 |
| | | | | S | NA | 96275390 | rs2549800 |
| | | | | I | A | 96276020 | rs2549801 |
| | | | | S | NA | 96297394 | rs2617434 |
| | | | | H/I | T | 96293411 | rs2617447 |
| | | | | I | T | 96372034 | rs27289 |
| | | | | H/I | A | 96375844 | rs27290 |
| | | | | S | NA | 96376026 | rs27291 |
| | | | | S | NA | 96382934 | rs27293 |
| | | | | H | G | 96383016 | rs27294 |
| | | | | H/I | T | 96387382 | rs27296 |
| | | | | S | NA | 96388556 | rs27298 |
| | | | | S | NA | 96388807 | rs27299 |
| | | | | H | T | 96389163 | rs27300 |
| | | | | I | A | 96399506 | rs27302 |
| | | | | S | NA | 96359090 | rs27305 |
| | | | | H/I | T | 96360314 | rs27306 |
| | | | | H/I | G | 96364261 | rs27307 |
| | | | | H | G | 96366372 | rs27397 |
| | | | | H/I | C | 96356722 | rs27436 |
| | | | | H | G | 96365029 | rs27613 |
| | | | | H/I | G | 96299054 | rs2762 |
| | | | | I | C | 96387089 | rs27621 |
| | | | | H/I | G | 96369308 | rs27659 |
| | | | | I | T | 96389819 | rs27712 |
| | | | | H | G | 96385668 | rs27747 |
| | | | | I | G | 96381359 | rs27993 |
| | | | | I | T | 96365244 | rs27997 |
| | | | | H/I | T | 96278345 | rs2910686 |
| | | | | S | NA | 96277457 | rs2910688 |
| | | | | I | C | 96299979 | rs2910787 |
| | | | | S | NA | 96302151 | rs2910789 |
| | | | | I | T | 96302142 | rs2910792 |
| | | | | H | C | 96277835 | rs2927609 |
| | | | | S | NA | 96259970 | rs3096167 |
| | | | | S | NA | 96259968 | rs3096168 |
| | | | | I | A | 96382420 | rs31398 |
| | | | | S | NA | 96364859 | rs3214461 |
| | | | | S | NA | 96322341 | rs33918743 |
| | | | | S | NA | 96268622 | rs33934033 |
| | | | | S | NA | 96243448 | rs34037881 |
| | | | | S | NA | 96353305 | rs34323164 |
| | | | | S | NA | 96354765 | rs34340727 |
| | | | | S | NA | 96258006 | rs34701361 |
| | | | | S | NA | 96306710 | rs34815125 |
| | | | | S | NA | 96314264 | rs34962665 |
| | | | | S | NA | 96344773 | rs35304156 |
| | | | | S | NA | 96357125 | rs35475916 |
| | | | | S | NA | 96371146 | rs35562078 |
| | | | | S | NA | 96301058 | rs35929998 |
| | | | | S | NA | 96314613 | rs36019589 |
| | | | | H/I | T | 96254184 | rs3734015 |
| | | | | H/I | G | 96342514 | rs3797796 |
| | | | | I | G | 96378979 | rs38029 |
| | | | | H/I | T | 96379440 | rs38030 |
| | | | | S | NA | 96381204 | rs38031 |
| | | | | H/I | T | 96347643 | rs38032 |
| | | | | H/I | A | 96347892 | rs38033 |
| | | | | H/I | C | 96348175 | rs38034 |
| | | | | H/I | C | 96349036 | rs38035 |
| | | | | H | A | 96349259 | rs38036 |
| | | | | I | G | 96353419 | rs38040 |
| | | | | H | G | 96356058 | rs38041 |
| | | | | H/I | A | 96361106 | rs38042 |
| | | | | H/I | G | 96362547 | rs38043 |
| | | | | S | NA | 96363546 | rs38044 |
| | | | | H | T | 96260289 | rs3849749 |
| | | | | H/I | A | 96260334 | rs3849750 |
| | | | | S | NA | 96320877 | rs3909451 |
| | | | | H/I | G | 96390210 | rs39602 |
| | | | | S | NA | 96260693 | rs3985004/rs33912722* |
| | | | | S | NA | 96260692 or | rs3985004 or |
| | | | | | | 96260693 | rs33912722* |
| | | | | S | NA | 96363405 | rs42983 |
| | | | | S | NA | 96357127 | rs430827 |
| | | | | H | A | 96318909 | rs4360063 |
| | | | | H/I | T | 96254981 | rs4869314 |
| | | | | H | A | 96255028 | rs4869315 |
| | | | | S | NA | 96259011 | rs5869737 |
| | | | | S | NA | 96278700 | rs5869740 |
| | | | | H/I | G | 96251952 | rs6556942 |
| | | | | S | NA | 96260062 | rs6859160 |
| | | | | S | NA | 96260071 | rs6859168 |
| | | | | S | NA | 96249932 | rs6868302 |
| | | | | I | C | 96307418 | rs6871162 |
| | | | | S | NA | 96260108 | rs6873441 |
| | | | | S | NA | 96260131 | rs6874656 |
| | | | | S | NA | 96345686 | rs6879678 |
| | | | | I | G | 96303477 | rs6887500 |
| | | | | H | G | 96290756 | rs716848 |
| | | | | H | G | 96333368 | rs7700332 |
| | | | | I | G | 96315986 | rs7703341 |
| | | | | H/I | A | 96313894 | rs7713127 |
| | | | | H | C | 96318762 | rs7716222 |
| | | | | H | G | 96312042 | rs7719705 |
| | | | | I | T | 96345247 | rs7722694 |
| | | | | S | NA | 96306799 | rs7726445 |
| | | | | H/I | G | 96314716 | rs7731592 |
| | | | | I | C | 96311577 | rs7733312 |
| | | | | H | G | 96315467 | rs7736466 |
| | | | | I | A | 96397921 | rs9127 |
| | | | | I | T | 96346342 | rs9314181 |
| LNPEP | AG | 96356058 | rs38041 | S | NA | 96346167 | rs10038651 |
| | (G) | | | | | | |
| | | | | H/I | C | 96346251 | rs10044354 |
| | | | | H/I | A | 96305246 | rs10051637 |
| | | | | H | T | 96323283 | rs10058476 |
| | | | | S | NA | 96309577 | rs10061936 |
| | | | | I | G | 96310559 | rs10069361 |
| | | | | H/I | G | 96326898 | rs10071975 |
| | | | | H/I | G | 96280573 | rs1019503 |
| | | | | S | NA | 96251278 | rs10434708 |
| | | | | I | C | 96251530 | rs10434709 |
| | | | | I | T | 96345363 | rs10476696 |
| | | | | S | NA | 96276415 | rs10537702 |
| | | | | S | NA | 96276948 | rs10546363 |
| | | | | H/I | T | 96271195 | rs1056893 |
| | | | | S | NA | 96284454 | rs10592692 |
| | | | | S | NA | 96255974 | rs10707238 |
| | | | | I | A | 96247182 | rs11135482 |
| | | | | I | G | 96247321 | rs11135483 |
| | | | | I | G | 96247645 | rs11135484 |
| | | | | S | NA | 96312725 | rs11135485 |
| | | | | S | NA | 96357847 | rs11311774 |
| | | | | S | NA | 96370825 | rs11414909 |
| | | | | I | A | 96247097 | rs11750025 |
| | | | | H | C | 96291635 | rs1216565 |
| | | | | S | NA | 96289812 | rs1216566 |
| | | | | S | NA | 96289595 | rs1216567 |
| | | | | S | NA | 96289402 | rs1216568 |
| | | | | S | NA | 96288290 | rs1216569 |
| | | | | S | NA | 96287473 | rs1216570 |
| | | | | I | T | 96246767 | rs12189125 |
| | | | | I | A | 96279965 | rs1230381 |
| | | | | I | T | 96280110 | rs1230382 |
| | | | | H | T | 96254538 | rs12516666 |
| | | | | H | A | 96333392 | rs12716486 |
| | | | | I | C | 96247776 | rs13167902 |
| | | | | S | NA | 96304809 | rs13170029 |
| | | | | I | A | 96248383 | rs13189819 |
| | | | | S | NA | 96321566 | rs13358339 |
| | | | | H | G | 96278559 | rs1363907 |
| | | | | S | NA | 96278860 | rs1363908 |
| | | | | H/I | A | 96319572 | rs1363974 |
| | | | | S | NA | 96274642 | rs1363975 |
| | | | | S | NA | 96274551 | rs1363976 |
| | | | | I | A | 96274463 | rs1363977 |
| | | | | H/I | T | 96324514 | rs1423357 |
| | | | | I | A | 96299523 | rs1423566 |
| | | | | H | G | 96310648 | rs1477364 |
| | | | | H | A | 96339986 | rs1544777 |
| | | | | I | T | 96329454 | rs1559267 |
| | | | | I | G | 96249877 | rs1559354 |
| | | | | H/I | T | 96252451 | rs1559355 |
| | | | | S | NA | 96252485 | rs1559356 |
| | | | | S | NA | 96252486 | rs1559357 |
| | | | | S | NA | 96268737 | rs17087165 |
| | | | | I | T | 96263169 | rs171647 |
| | | | | H | T | 96377824 | rs18059 |
| | | | | S | NA | 96278754 | rs1820148 |
| | | | | S | NA | 96332914 | rs1820149 |
| | | | | H/I | G | 96262870 | rs187265 |
| | | | | I | C | 96260628 | rs193993 |
| | | | | H | C | 96252291 | rs1974871 |
| | | | | H/I | G | 96294618 | rs1981846 |
| | | | | S | NA | 96260377 | rs2042383 |
| | | | | H | G | 96273749 | rs2042385 |
| | | | | S | NA | 40328876 | rs210687 |
| | | | | H/I | A | 96340258 | rs2113050 |
| | | | | H | A | 96259206 | rs2113189 |
| | | | | I | A | 96262074 | rs2113190 |
| | | | | I | C | 96272094 | rs2113191 |
| | | | | H/I | G | 96343901 | rs2161548 |
| | | | | H/I | T | 96258562 | rs2161657 |
| | | | | H/I | C | 96265401 | rs2161658 |
| | | | | H/I | A | 96255506 | rs2247650 |
| | | | | I | G | 96261652 | rs2248374 |
| | | | | H/I | A | 96274871 | rs2255546 |
| | | | | H/I | T | 96275079 | rs2255633 |
| | | | | H | T | 96275107 | rs2255634 |
| | | | | H | G | 96275134 | rs2255637 |
| | | | | H/I | T | 96250335 | rs2278018 |
| | | | | I | A | 96251008 | rs2278019 |
| | | | | H/I | A | 96263082 | rs2287988 |
| | | | | S | NA | 96247939 | rs2303208 |
| | | | | I | T | 96247941 | rs2303209 |
| | | | | H/I | C | 96319685 | rs2351010 |
| | | | | S | NA | 96277431 | rs2351011 |
| | | | | H/I | A | 96396635 | rs248215 |
| | | | | H/I | C | 96260794 | rs251339 |
| | | | | S | NA | 96262168 | rs251340 |
| | | | | I | T | 96262599 | rs251342 |
| | | | | S | NA | 96283585 | rs251343 |
| | | | | H | G | 96284683 | rs251344 |
| | | | | I | A | 96298789 | rs2548225 |
| | | | | I | G | 96301169 | rs2548516 |
| | | | | S | NA | 96276759 | rs2548520 |
| | | | | S | NA | 96276684 | rs2548521 |
| | | | | I | T | 96276213 | rs2548522 |
| | | | | H/I | A | 96272696 | rs2548523 |
| | | | | H/I | A | 96272357 | rs2548524 |
| | | | | I | G | 96271373 | rs2548526 |
| | | | | H | G | 96270341 | rs2548527 |
| | | | | H/I | G | 96265976 | rs2548529 |
| | | | | H | G | 96265683 | rs2548530 |
| | | | | H | A | 96264334 | rs2548532 |
| | | | | H/I | C | 96264157 | rs2548533 |
| | | | | I | T | 96259364 | rs2548534 |
| | | | | I | C | 96258455 | rs2548535 |
| | | | | H | T | 96258158 | rs2548536 |
| | | | | I | G | 96257978 | rs2548537 |
| | | | | H/I | T | 96257898 | rs2548538 |
| | | | | H | A | 96257260 | rs2548539 |
| | | | | H | T | 96255934 | rs2548540 |
| | | | | H | T | 96255878 | rs2549781 |
| | | | | H/I | T | 96256756 | rs2549782 |
| | | | | H | C | 96257128 | rs2549783 |
| | | | | H | T | 96257276 | rs2549784 |
| | | | | I | T | 96258042 | rs2549785 |
| | | | | S | NA | 96265593 | rs2549787 |
| | | | | I | G | 96266142 | rs2549788 |
| | | | | S | NA | 96268026 | rs2549789 |
| | | | | H | C | 96268168 | rs2549790 |
| | | | | H/I | A | 96268198 | rs2549791 |
| | | | | H | T | 96270305 | rs2549794 |
| | | | | H/I | G | 96270394 | rs2549795 |
| | | | | H/I | T | 96271099 | rs2549796 |
| | | | | H/I | G | 96271274 | rs2549797 |
| | | | | S | NA | 96271659 | rs2549798 |
| | | | | S | NA | 96271666 | rs2549799 |
| | | | | S | NA | 96275390 | rs2549800 |
| | | | | I | A | 96276020 | rs2549801 |
| | | | | S | NA | 96297394 | rs2617434 |
| | | | | H | T | 96293411 | rs2617447 |
| | | | | I | T | 96372034 | rs27289 |
| | | | | H/I | A | 96375844 | rs27290 |
| | | | | S | NA | 96376026 | rs27291 |
| | | | | I | G | 96382736 | rs27292 |
| | | | | S | NA | 96382934 | rs27293 |
| | | | | H | G | 96383016 | rs27294 |
| | | | | H/I | T | 96387382 | rs27296 |
| | | | | S | NA | 96388556 | rs27298 |
| | | | | S | NA | 96388807 | rs27299 |
| | | | | H | T | 96389163 | rs27300 |
| | | | | I | A | 96399506 | rs27302 |
| | | | | S | NA | 96359090 | rs27305 |
| | | | | H | T | 96360314 | rs27306 |
| | | | | H | G | 96364261 | rs27307 |
| | | | | H/I | G | 96366372 | rs27397 |
| | | | | H/I | C | 96356722 | rs27436 |
| | | | | H | G | 96365029 | rs27613 |
| | | | | H/I | G | 96299054 | rs2762 |
| | | | | I | C | 96387089 | rs27621 |
| | | | | H/I | G | 96369308 | rs27659 |
| | | | | H | G | 96371495 | rs27711 |
| | | | | H | G | 96385668 | rs27747 |
| | | | | I | G | 96381359 | rs27993 |
| | | | | H/I | T | 96278345 | rs2910686 |
| | | | | S | NA | 96277457 | rs2910688 |
| | | | | I | C | 96299979 | rs2910787 |
| | | | | S | NA | 96302151 | rs2910789 |
| | | | | I | T | 96302142 | rs2910792 |
| | | | | H | C | 96277835 | rs2927609 |
| | | | | S | NA | 96259970 | rs3096167 |
| | | | | S | NA | 96259968 | rs3096168 |
| | | | | I | A | 96382420 | rs31398 |
| | | | | S | NA | 96364859 | rs3214461 |
| | | | | S | NA | 96322341 | rs33918743 |
| | | | | S | NA | 96268622 | rs33934033 |
| | | | | S | NA | 96243448 | rs34037881 |
| | | | | S | NA | 96353305 | rs34323164 |
| | | | | S | NA | 96354765 | rs34340727 |
| | | | | S | NA | 96258006 | rs34701361 |
| | | | | S | NA | 96306710 | rs34815125 |
| | | | | S | NA | 96314264 | rs34962665 |
| | | | | S | NA | 96344773 | rs35304156 |
| | | | | S | NA | 96357125 | rs35475916 |
| | | | | S | NA | 96371146 | rs35562078 |
| | | | | S | NA | 96301058 | rs35929998 |
| | | | | S | NA | 96314613 | rs36019589 |
| | | | | H/I | T | 96254184 | rs3734015 |
| | | | | H/I | G | 96342514 | rs3797796 |
| | | | | I | G | 96378979 | rs38029 |
| | | | | H/I | T | 96379440 | rs38030 |
| | | | | S | NA | 96381204 | rs38031 |
| | | | | H | T | 96347643 | rs38032 |
| | | | | H/I | A | 96347892 | rs38033 |
| | | | | H/I | C | 96348175 | rs38034 |
| | | | | H/I | C | 96349036 | rs38035 |
| | | | | H | A | 96349259 | rs38036 |
| | | | | I | G | 96353419 | rs38040 |
| | | | | H/I | A | 96361106 | rs38042 |
| | | | | H/I | G | 96362547 | rs38043 |
| | | | | S | NA | 96363546 | rs38044 |
| | | | | H | T | 96260289 | rs3849749 |
| | | | | H/I | A | 96260334 | rs3849750 |
| | | | | S | NA | 96320877 | rs3909451 |
| | | | | H/I | G | 96390210 | rs39602 |
| | | | | S | NA | 96260692 or | rs3985004 or |
| | | | | | | 96260693 | rs33912722* |
| | | | | S | NA | 96363405 | rs42983 |
| | | | | S | NA | 96357127 | rs430827 |
| | | | | H | A | 96318909 | rs4360063 |
| | | | | H/I | T | 96254981 | rs4869314 |
| | | | | H | A | 96255028 | rs4869315 |
| | | | | H | G | 96259219 | rs4869316 |
| | | | | S | NA | 96259011 | rs5869737 |
| | | | | S | NA | 96278700 | rs5869740 |
| | | | | H | G | 96251952 | rs6556942 |
| | | | | S | NA | 96260062 | rs6859160 |
| | | | | S | NA | 96260071 | rs6859168 |
| | | | | S | NA | 96249932 | rs6868302 |
| | | | | I | C | 96307418 | rs6871162 |
| | | | | S | NA | 96260108 | rs6873441 |
| | | | | S | NA | 96260131 | rs6874656 |
| | | | | S | NA | 96345686 | rs6879678 |
| | | | | I | G | 96303477 | rs6887500 |
| | | | | H | G | 96290756 | rs716848 |
| | | | | H | G | 96333368 | rs7700332 |
| | | | | I | G | 96315986 | rs7703341 |
| | | | | H/I | A | 96313894 | rs7713127 |
| | | | | H | C | 96318762 | rs7716222 |
| | | | | H | G | 96312042 | rs7719705 |
| | | | | I | T | 96345247 | rs7722694 |
| | | | | S | NA | 96306799 | rs7726445 |
| | | | | H/I | G | 96314716 | rs7731592 |
| | | | | H | G | 96315467 | rs7736466 |
| | | | | I | A | 96397921 | rs9127 |
| | | | | I | T | 96346342 | rs9314181 |
| LNPEP | GA | 96305246 | rs10051637 | S | NA | 96346167 | rs10038651 |
| | (A) | | | | | | |
| | | | | H/I | C | 96346251 | rs10044354 |
| | | | | H | T | 96323283 | rs10058476 |
| | | | | S | NA | 96309577 | rs10061936 |
| | | | | I | G | 96310559 | rs10069361 |
| | | | | H/I | G | 96326898 | rs10071975 |
| | | | | H/I | G | 96280573 | rs1019503 |
| | | | | S | NA | 96251278 | rs10434708 |
| | | | | I | C | 96251530 | rs10434709 |
| | | | | I | G | 96298936 | rs1046395 |
| | | | | I | T | 96345363 | rs10476696 |
| | | | | S | NA | 96276415 | rs10537702 |
| | | | | S | NA | 96276948 | rs10546363 |
| | | | | H/I | T | 96271195 | rs1056893 |
| | | | | S | NA | 96284454 | rs10592692 |
| | | | | S | NA | 96255974 | rs10707238 |
| | | | | I | G | 96247321 | rs11135483 |
| | | | | I | G | 96247645 | rs11135484 |
| | | | | S | NA | 96312725 | rs11135485 |
| | | | | S | NA | 96357847 | rs11311774 |
| | | | | S | NA | 96370825 | rs11414909 |
| | | | | I | A | 96247097 | rs11750025 |
| | | | | H | C | 96291635 | rs1216565 |
| | | | | S | NA | 96289812 | rs1216566 |
| | | | | S | NA | 96289595 | rs1216567 |
| | | | | S | NA | 96289402 | rs1216568 |
| | | | | S | NA | 96288290 | rs1216569 |
| | | | | S | NA | 96287473 | rs1216570 |
| | | | | I | T | 96246767 | rs12189125 |
| | | | | H | G | 96237497 | rs1230358 |
| | | | | I | A | 96279965 | rs1230381 |
| | | | | I | T | 96280110 | rs1230382 |
| | | | | H | T | 96254538 | rs12516666 |
| | | | | H | A | 96333392 | rs12716486 |
| | | | | I | C | 96247776 | rs13167902 |
| | | | | S | NA | 96304809 | rs13170029 |
| | | | | I | A | 96248383 | rs13189819 |
| | | | | S | NA | 96321566 | rs13358339 |
| | | | | H/I | G | 96278559 | rs1363907 |
| | | | | S | NA | 96278860 | rs1363908 |
| | | | | H/I | A | 96319572 | rs1363974 |
| | | | | S | NA | 96274642 | rs1363975 |
| | | | | S | NA | 96274551 | rs1363976 |
| | | | | I | A | 96274463 | rs1363977 |
| | | | | H/I | T | 96324514 | rs1423357 |
| | | | | I | A | 96299523 | rs1423566 |
| | | | | H | G | 96310648 | rs1477364 |
| | | | | H | A | 96339986 | rs1544777 |
| | | | | I | T | 96329454 | rs1559267 |
| | | | | I | G | 96249877 | rs1559354 |
| | | | | H/I | T | 96252451 | rs1559355 |
| | | | | S | NA | 96252485 | rs1559356 |
| | | | | S | NA | 96252486 | rs1559357 |
| | | | | S | NA | 96268737 | rs17087165 |
| | | | | I | T | 96263169 | rs171647 |
| | | | | H/I | T | 96377824 | rs18059 |
| | | | | S | NA | 96278754 | rs1820148 |
| | | | | S | NA | 96332914 | rs1820149 |
| | | | | H/I | G | 96262870 | rs187265 |
| | | | | I | C | 96260628 | rs193993 |
| | | | | H | C | 96252291 | rs1974871 |
| | | | | H/I | G | 96294618 | rs1981846 |
| | | | | S | NA | 96260377 | rs2042383 |
| | | | | H | G | 96273749 | rs2042385 |
| | | | | S | NA | 40328876 | rs210687 |
| | | | | H/I | A | 96340258 | rs2113050 |
| | | | | H | A | 96259206 | rs2113189 |
| | | | | I | A | 96262074 | rs2113190 |
| | | | | I | C | 96272094 | rs2113191 |
| | | | | H/I | G | 96343901 | rs2161548 |
| | | | | H/I | T | 96258562 | rs2161657 |
| | | | | H/I | C | 96265401 | rs2161658 |
| | | | | H/I | A | 96255506 | rs2247650 |
| | | | | I | G | 96261652 | rs2248374 |
| | | | | H | A | 96274871 | rs2255546 |
| | | | | H/I | T | 96275079 | rs2255633 |
| | | | | H | T | 96275107 | rs2255634 |
| | | | | H | G | 96275134 | rs2255637 |
| | | | | H/I | T | 96250335 | rs2278018 |
| | | | | I | A | 96251008 | rs2278019 |
| | | | | H/I | A | 96263082 | rs2287988 |
| | | | | S | NA | 96247939 | rs2303208 |
| | | | | I | T | 96247941 | rs2303209 |
| | | | | H/I | C | 96319685 | rs2351010 |
| | | | | S | NA | 96277431 | rs2351011 |
| | | | | H/I | A | 96396635 | rs248215 |
| | | | | H/I | C | 96260794 | rs251339 |
| | | | | S | NA | 96262168 | rs251340 |
| | | | | I | T | 96262599 | rs251342 |
| | | | | S | NA | 96283585 | rs251343 |
| | | | | H | G | 96284683 | rs251344 |
| | | | | I | A | 96298789 | rs2548225 |
| | | | | I | G | 96301169 | rs2548516 |
| | | | | I | G | | rs2548517 |
| | | | | S | NA | 96276759 | rs2548520 |
| | | | | S | NA | 96276684 | rs2548521 |
| | | | | I | T | 96276213 | rs2548522 |
| | | | | H/I | A | 96272696 | rs2548523 |
| | | | | H/I | A | 96272357 | rs2548524 |
| | | | | I | G | 96271373 | rs2548526 |
| | | | | H | G | 96270341 | rs2548527 |
| | | | | H/I | G | 96265976 | rs2548529 |
| | | | | H/I | G | 96265683 | rs2548530 |
| | | | | H | A | 96264334 | rs2548532 |
| | | | | H/I | C | 96264157 | rs2548533 |
| | | | | I | T | 96259364 | rs2548534 |
| | | | | I | C | 96258455 | rs2548535 |
| | | | | H | T | 96258158 | rs2548536 |
| | | | | I | G | 96257978 | rs2548537 |
| | | | | H/I | T | 96257898 | rs2548538 |
| | | | | H | A | 96257260 | rs2548539 |
| | | | | H | T | 96255934 | rs2548540 |
| | | | | H | T | 96255878 | rs2549781 |
| | | | | H/I | T | 96256756 | rs2549782 |
| | | | | H/I | C | 96257128 | rs2549783 |
| | | | | H | T | 96257276 | rs2549784 |
| | | | | I | T | 96258042 | rs2549785 |
| | | | | S | NA | 96265593 | rs2549787 |
| | | | | I | G | 96266142 | rs2549788 |
| | | | | S | NA | 96268026 | rs2549789 |
| | | | | H | C | 96268168 | rs2549790 |
| | | | | H/I | A | 96268198 | rs2549791 |
| | | | | H/I | T | 96270305 | rs2549794 |
| | | | | H/I | G | 96270394 | rs2549795 |
| | | | | H/I | T | 96271099 | rs2549796 |
| | | | | H/I | G | 96271274 | rs2549797 |
| | | | | S | NA | 96271659 | rs2549798 |
| | | | | S | NA | 96271666 | rs2549799 |
| | | | | S | NA | 96275390 | rs2549800 |
| | | | | I | A | 96276020 | rs2549801 |
| | | | | S | NA | 96297394 | rs2617434 |
| | | | | H/I | T | 96293411 | rs2617447 |
| | | | | I | T | 96372034 | rs27289 |
| | | | | H/I | A | 96375844 | rs27290 |
| | | | | S | NA | 96376026 | rs27291 |
| | | | | I | G | 96382736 | rs27292 |
| | | | | S | NA | 96382934 | rs27293 |
| | | | | H | G | 96383016 | rs27294 |
| | | | | H/I | T | 96387382 | rs27296 |
| | | | | S | NA | 96388556 | rs27298 |
| | | | | S | NA | 96388807 | rs27299 |
| | | | | H | T | 96389163 | rs27300 |
| | | | | I | A | 96399506 | rs27302 |
| | | | | S | NA | 96359090 | rs27305 |
| | | | | H/I | T | 96360314 | rs27306 |
| | | | | H/I | G | 96364261 | rs27307 |
| | | | | H/I | G | 96366372 | rs27397 |
| | | | | H/I | C | 96356722 | rs27436 |
| | | | | H | G | 96365029 | rs27613 |
| | | | | H/I | G | 96299054 | rs2762 |
| | | | | I | C | 96387089 | rs27621 |
| | | | | H/I | G | 96369308 | rs27659 |
| | | | | H/I | G | 96371495 | rs27711 |
| | | | | I | T | 96389819 | rs27712 |
| | | | | H | G | 96385668 | rs27747 |
| | | | | I | G | 96381359 | rs27993 |
| | | | | I | T | 96365244 | rs27997 |
| | | | | H/I | T | 96278345 | rs2910686 |
| | | | | S | NA | 96277457 | rs2910688 |
| | | | | I | C | 96299979 | rs2910787 |
| | | | | S | NA | 96302151 | rs2910789 |
| | | | | I | T | 96302142 | rs2910792 |
| | | | | H | C | 96277835 | rs2927609 |
| | | | | S | NA | 96259970 | rs3096167 |
| | | | | S | NA | 96259968 | rs3096168 |
| | | | | I | A | 96382420 | rs31398 |
| | | | | S | NA | 96364859 | rs3214461 |
| | | | | S | NA | 96322341 | rs33918743 |
| | | | | S | NA | 96268622 | rs33934033 |
| | | | | S | NA | 96243448 | rs34037881 |
| | | | | S | NA | 96353305 | rs34323164 |
| | | | | S | NA | 96354765 | rs34340727 |
| | | | | S | NA | 96258006 | rs34701361 |
| | | | | S | NA | 96306710 | rs34815125 |
| | | | | S | NA | 96314264 | rs34962665 |
| | | | | S | NA | 96344773 | rs35304156 |
| | | | | S | NA | 96357125 | rs35475916 |
| | | | | S | NA | 96371146 | rs35562078 |
| | | | | S | NA | 96301058 | rs35929998 |
| | | | | S | NA | 96314613 | rs36019589 |
| | | | | H/I | T | 96254184 | rs3734015 |
| | | | | H/I | G | 96342514 | rs3797796 |
| | | | | I | G | 96378979 | rs38029 |
| | | | | H/I | T | 96379440 | rs38030 |
| | | | | S | NA | 96381204 | rs38031 |
| | | | | H/I | T | 96347643 | rs38032 |
| | | | | H/I | A | 96347892 | rs38033 |
| | | | | H/I | C | 96348175 | rs38034 |
| | | | | H/I | C | 96349036 | rs38035 |
| | | | | H | A | 96349259 | rs38036 |
| | | | | I | G | 96353419 | rs38040 |
| | | | | H/I | G | 96356058 | rs38041 |
| | | | | H/I | A | 96361106 | rs38042 |
| | | | | H/I | G | 96362547 | rs38043 |
| | | | | S | NA | 96363546 | rs38044 |
| | | | | H | T | 96260289 | rs3849749 |
| | | | | H/I | A | 96260334 | rs3849750 |
| | | | | S | NA | 96320877 | rs3909451 |
| | | | | H/I | G | 96390210 | rs39602 |
| | | | | S | NA | 96260692 or | rs3985004 or |
| | | | | | | 96260693 | rs33912722* |
| | | | | | NA | 96260693 | |
| | | | | S | NA | 96363405 | rs42983 |
| | | | | S | NA | 96357127 | rs430827 |
| | | | | H | A | 96318909 | rs4360063 |
| | | | | H/I | T | 96254981 | rs4869314 |
| | | | | H | A | 96255028 | rs4869315 |
| | | | | S | NA | 96259011 | rs5869737 |
| | | | | S | NA | 96278700 | rs5869740 |
| | | | | H/I | G | 96251952 | rs6556942 |
| | | | | S | NA | 96260062 | rs6859160 |
| | | | | S | NA | 96260071 | rs6859168 |
| | | | | S | NA | 96249932 | rs6868302 |
| | | | | I | C | 96307418 | rs6871162 |
| | | | | S | NA | 96260108 | rs6873441 |
| | | | | S | NA | 96260131 | rs6874656 |
| | | | | S | NA | 96345686 | rs6879678 |
| | | | | I | G | 96303477 | rs6887500 |
| | | | | H | G | 96290756 | rs716848 |
| | | | | H | G | 96333368 | rs7700332 |
| | | | | I | G | 96315986 | rs7703341 |
| | | | | H/I | A | 96313894 | rs7713127 |
| | | | | H | C | 96318762 | rs7716222 |
| | | | | H | G | 96312042 | rs7719705 |
| | | | | I | T | 96345247 | rs7722694 |
| | | | | S | NA | 96306799 | rs7726445 |
| | | | | H/I | G | 96314716 | rs7731592 |
| | | | | I | C | 96311577 | rs7733312 |
| | | | | H | G | 96315467 | rs7736466 |
| | | | | I | A | 96397921 | rs9127 |
| | | | | I | T | 96346342 | rs9314181 |
| AVPR1A | CT | 61837421 | rs10877970 | H | G | 61816874 | rs7972829 |
| | (C) | | | | | | |
| | | | | H | C | 61824913 | rs10784339 |
| | | | | H | T | 61827101 | rs3803107 |
| | | | | H | G | 61840232 | rs11836346 |
| | | | | H | A | 61844229 | rs7308008 |
| | | | | H | G | 61849214 | rs11835545 |
| | | | | H | A | 61851233 | rs7959001 |
| | | | | H | T | 61852342 | rs11832877 |
| | | | | H | C | 61853617 | rs10877977 |
| | | | | H | G | 61860197 | rs2201895 |
| | | | | H | T | 61862861 | rs7302323 |
| | | | | H | T | 61868529 | rs10877986 |
| | | | | H | A | 61884651 | rs2030106 |
| | | | | S | NA | 61824725 | rs10747983 |
| | | | | S | NA | 61833506 | rs10877969 |
| | | | | S | NA | 61834359 | rs7294536 |
| AVPR1A | AT | 61827101 | rs3803107 | H | G | 61816874 | rs7972829 |
| | (T) | | | | | | |
| | | | | H | C | 61824913 | rs10784339 |
| | | | | H | C | 61837421 | rs10877970 |
| | | | | H | G | 61840232 | rs11836346 |
| | | | | H | A | 61844229 | rs7308008 |
| | | | | H | G | 61849214 | rs11835545 |
| | | | | H | A | 61851233 | rs7959001 |
| | | | | H | T | 61852342 | rs11832877 |
| | | | | H | C | 61853617 | rs10877977 |
| | | | | H | T | 61862861 | rs7302323 |
| | | | | H | T | 61868529 | rs10877986 |
| | | | | H | A | 61884651 | rs2030106 |
| AVPR1A | CT | 61890334 | rs1495027 | H | T | 61807179 | rs10877962 |
| | (T) | | | | | | |
| | | | | H | T | 61830476 | rs1042615 |
| | | | | H | G | 61900977 | rs16856 |
| | | | | S | NA | 61825030 | rs36014760 |
| | | | | S | NA | 61826743 | rs11174811 |
| | | | | S | NA | 61828619 | rs34462214 |
| | | | | S | NA | 61831947 | rs3021529 |
| | | | | S | NA | 61833506 | rs10877969 |
| | | | | S | NA | 61834359 | rs7294536 |
| | | | | S | NA | 61824725 | rs10747983 |

A ‘*’ indicates that there is more than one RSID assigned to a single SNP.
NA as used above indicates that the LD allele with the information currently available to the inventors could not with any confidence be assigned without further routine analysis, due to the lack of suitable information currently available regarding the corresponding allele designations. However, it would be well within the abilities of a person of skill in the art to make LD allele designations for the NA polymorphisms using routine analysis.

It will be appreciated by a person of skill in the art that further linked polymorphic sites and combined polymorphic sites may be determined. A haplotype of vasopressin pathway associated genes can be created by assessing polymorphisms in vasopressin pathway-associated genes in normal subjects using a program that has an expectation maximization algorithm (i.e. PHASE). A constructed haplotype of vasopressin pathway associated genes may be used to find combinations of SNPs that are in LD with the tag SNPs (tSNPs) identified herein. Accordingly, the haplotype of an individual could be determined by genotyping other SNPs or other polymorphisms that are in LD with the tSNPs identified herein. Single polymorphic sites or combined polymorphic sites in LD may also be genotyped for assessing subject response to vasopressin receptor agonist treatment.

It will be appreciated by a person of skill in the art that the numerical designations of the positions of polymorphisms within a sequence are relative to the specific sequence. Also the same positions may be assigned different numerical designations depending on the way in which the sequence is numbered and the sequence chosen, as illustrated by the alternative numbering of the equivalent polymorphism (rs3803107), whereby the same polymorphism identified C/T at position 3536 of the NM_{—000706.3} (GI:33149325), which corresponds to position 201 of SEQ ID NO:9. Furthermore, sequence variations within the population, such as insertions or deletions, may change the relative position and subsequently the numerical designations of particular nucleotides at and around a polymorphic site.

Polymorphic sites in SEQ ID NO:1-10 are identified by their variant designation (i.e. M, W, Y, S, R, K, V, B, D, H or by “−” for a deletion, a “+” or for example “G” etc. for an insertion).

Polymorphic sites in SEQ ID NO:11-264 are identified by their allelic change (i.e. A, C, G, T or by “−” for a deletion, a “+” or for an insertion).

An “rs” prefix designates a SNP in the database is found at the NCBI SNP database (http://www.ncbi.nlm.nih.gov/entrez/query.fcgi?db=Snp). The “rs” numbers are the NCBI|rsSNP ID form.

TABLE 1C below shows the flanking sequences for a selection of vasopressin pathway associated gene SNPs providing their rs designations and corresponding SEQ ID NO designations. Each polymorphism is at position 201 within the flanking sequence, and identified in bold and underlined

| | | | |
|---|---|---|---|
| | | SEQ | |
| | | ID | |
| GENE | SNP | NO: | FLANKING SEQUENCE |

| | | | |
|---|---|---|---|
| LNPEP | rs18059 | 1 | TTAAGTTAGATGATTTTCTGAGGTCTCTTCTAATGGTTAAGATTTTTATC |
| | | | ATTTTCTATTTCATAAGGCTTTCAGCTAGCAGCCTTAATAAAAACCAGTG |
| | | | CCTGGAACATGACCTGGCCTGTAGTGACACTCAGTAAACGTTGAGTGAAT |
| | | | AAATGATTGAAACACACCAGAAACAAGTGCATTTGAGTGCTTTTACACAC |
| | | | YGTGCTTAGTGCTTAATGTAGATTACCTCATTTAATTATCACACAGTGCC |
| | | | AAGGTAGATATTTCTACCCCCATTAAATAAACGAGGAGACGGAATAGCTT |
| | | | CTTTAAAGTCACTTACCTAGTAAGTGATAAAGCTGAAATTCAAACCCAGA |
| | | | TAAATTTCACTCCAAAGACTTCTGTTTCTGTTATATTGCTATTTGTAAAA |
| | | | TCAATTTGTGTCCTAGCAACGTCGTCTTTCCAGGATACCTTTAGAAAAAT |
| | | | TAAAGCTTTCTTCTTGTCATATTCTTTTGAAAAGCTTGCAGACCATATAT |
| | | | TTAAGGTTTCAAGTGACTGGCCCACATCTAGTTGTTCTCCTAAAAATGAA |
| | | | ATTGTCAACTTAAGAGA |
| LNPEP | rs27711 | 2 | TTTTTTCTATTCTCAAAAGAAAGGTAGCAGAGAGGGTGACTTCAGGCTTC |
| | | | TTTTATGCTGTAATACTTTAGTATAGTGTATTATTTTGATGCTTGATGGT |
| | | | TGGTTAAATCTTTAATATATTTTCTTTCTTTTTTTAAATATATTTTCATG |
| | | | TGTTCTAATTCAAGGGTTGTTGGGTTAGTTCATTAGTTCAGTTGTATATA |
| | | | RGAGTTATGTTTGGTCAATGTATTTGTCTCCTTTTCTCACATACATGAGT |
| | | | TTTGAACAATTAGTATTTATTGTGCACAAGAAATGCTGATGGGGCCATTT |
| | | | TTCCAGTTTACATATTGAGGAATTATATTTTTTAAAGTTTCCCTCTTCCC |
| | | | TTTCTTCCTCCCTCTCTCTCTCTCTTTCTTATCCACATTTTACTCAGACC |
| | | | TAAGATCTTTAACTATAGGAGATTTTCGTATTAAATCTAATGCAAAACAT |
| | | | TCATGTTT |
| LNPEP | rs38041 | 3 | TGTGGGGAAGAATCTCTTTCCCTAAGTTGCACCCTTCTGACAACTCAAAC |
| | | | TGTAGCTGTCAGGGCTGGATTTTTTTTTTTTTTCATCTCCTGCCGGAATG |
| | | | GGGTTCTCTGTTAATTTTGGAGAGGGGGTTTCTGAGAAAATGGCAAAGGG |
| | | | TACTGTTTGTATGACATGGAGAGAAAGAAAGAAAATTATATGGGTACATA |
| | | | RCACCCCCATTCTTCCCTAACACCTTGTCTCTATTTTGCCCTAGATGAGG |
| | | | TGCTTAACTAGCATTGGGTATGGTTTGGGTGATGTCATGACAGTGGCAGG |
| | | | ATATGAATAGGATGTATTCTGGTCAGTTTATTTTCTACATCAAACACCTT |
| | | | ATATGAATCTAGCCTTTGTGAAGACTTCATGACAAGCTGGCATATGAGCA |
| LNPEP | rs10051637 | 4 | TGGCCAGCCTACTATTCTTTATAGCCTGGCTTTGCTTCACTTTTCTACTG |
| | | | GTGCTTGTGATAGAACAATGAACCAATTAATTTTTTTAAAATTCCATCCT |
| | | | TAACATGTAAATGAGGAGGAAACCAGGTCATTTGCCAAATAAGGAAAATT |
| | | | CAAGCTTCCAAGGGAGTTTCAAAAAACAATGGAGGATCAAGTTCAATTGT |
| | | | RGGAGACTTTTTGAAATTCTTTTTCTTCTAATACATATTGCTTAATGAAG |
| | | | GTACTCCTGGGCATTCCACATATTTCAAAAATGTAGTCACTGAACCAGAA |
| | | | CTTGAATCAGTTGTCTGAATTTCTCTGGATTGTGGGGCTCAGAGTCTTCT |
| | | | CCAGCCAATGATCTGGGGTGAAGGAAGTTAAAGAAGGCTTCTTCAACTGA |
| AVP | rs1410713 | 5 | GCTATTAGATCTAATAAGTACATTTAGCAAGATCACAGGGTACAAAGTCA |
| | | | ACAATAATTCATAATTCTATATACTAACAATAACTACTTGGAAATTAAAT |
| | | | TTTAAAACACAGTACTATTTAAAATAGTGTGAAAAATATGAAATCTTTTG |
| | | | GGTAGAAATCTTACATAATATATACAAATTCTATATGCTGAACTAATAAA |
| | | | MTGCTGATGGAAGAAATAGAAGACCTGAATAACTGGAGAGATATATTTGT |
| | | | TTATTGATTTGGAAGACTCATCAGAGTAAAGATGTCAGTTCTCCCCAAAT |
| | | | TGATTGAAAGATCCAACACAATTCTAATTAAACTCCCAATAGGATTTTTT |
| | | | TGTAGAAATCATTAAGTTGATTCTAAAATTTATACAGAATGACAAAGGAA |
| | | | CTAGAATAGCCAAAACAATTTTGAAAAAGAACAAAGTTGGAAAAGCCACA |
| | | | CAACCTGATAAAGTTATCATATTCAAAATAGTATAATATTATAGAAAGGA |
| | | | TAGATCTAGGCCAGGTGCGGTGGCTCACGCCTGTAATCCCAGCACTTTGG |
| AVP | rs857240 | 6 | CAGGCCTGGCTGAAATTCAGGGATGGCATCTAGGGCTTCCCCACTCCTGT |
| | | | CTGGTACCCTCCACATGTCTGAGTGTCCCTCCTTGTGGCAAGGGGACAGC |
| | | | CACAAAATGGGTTCCCTCTTCTGAGCCTCTCCGCTCTCAGGGAGGAGAAA |
| | | | CCTGCCCAGAGTCCCCACCCCTAAATCCCTCCAGACTGGACAAGCACCAC |
| | | | YAGCCGGCTGCCTTCTTTGGGTTAGGCCAGCCAAAGCTCACCCCTAAGAT |
| | | | TAGGTGTGCTCACAGGCCCCTGACAAAATGCGGTCCTGTTGGTGAGGAAG |
| | | | AGGAGGGACGGGACTGGCTGCTGGGTCAGTAACTGGGGTATTTGTCCCCG |
| | | | GCCCCAGGCTGGAAGGCATTGGTAGACTTGTACAGATCACTTCACTTGTG |
| | | | GGGACCCTGTGGCACAGAGAGACCCCGTGGCTTGTCCGGGACCACACAGC |
| | | | TAAGCCGGGCAGAACTACTGAGCGAGGAGCCTATCAGTCCTGGTTCCCAA |
| AVP | rs857242 | 7 | CCAGAGGAAGGCCAGCTGCAAACCACTGACCCCAATGTCCGGCATCTGAG |
| | | | GGACGGACACGCCCAGGGGTCAAGAGAACGGAGCCTGGGAGTGGCATCCA |
| | | | CAGGGTCTGCTGTAGGCGACTCCAGTGCCTTCCTCTTGATCCCTCTGCTC |
| | | | AGGTGCCTACTCCAGGGAGGGGCTGGCTTTTTGGATTAGGTTGGATGATG |
| | | | MCCATCCTCAAGTGTCTGAATAAAGCTCCTTCAGAGTGAATGCAATGGAG |
| | | | AAAGGGTAGTGCCTTGAGAGGATCTCAGGATGATAGTAGGAAGGGAAATA |
| | | | AATGCTGTAAAGCTAAGCAGCCCTCACCCCCACAAATCCACTGAGATCTT |
| | | | TTCTTTTCTTTTAGAGAGGGTCTCACTCTATTGCCCAGGCTGGGGGGCAG |
| | | | TGGCACAAACACAGCTCGCTGCAGCCTTCACCTCCTGGGCTCAAGCGATC |
| | | | CTCCCATCTCAGCCTCCTGAGTAGCTGGGACTACAGACGTGTGCCACCAT |
| | | | GCCCAGCTAATTACGTTACCCAGGCTGGTCTTGAACTCCTGGGCTCGAAC |
| AVPR1A | rs10877970 | 8 | ATGGCTTTTTAAAATTTAAAATCATTTAAATGGTTGAGTTTAAGACTTTT |
| | | | GCTCCTAATGAATTCATATTCATTTGGGTGTTCTGCATCTTCATGGTCAG |
| | | | CAGTTTTGCTATCCTGTCTAAATTTGATCATCAAGACTCATTCTTCCAGC |
| | | | ATGCTGGCAACATTGAGACTACCTCTGTGTATTCATTAATTTGTTTTTCA |
| | | | YGAGTGAAAAGGTTTGCATTGTTTGAAGGGTGCTGAACAAAGTTGTGATA |
| | | | CTATAATTTTTTGTTTATCTGCTGTGAATACTATTTATTAGAATTTTTAA |
| | | | ATACTTATTTGCCTCTATTTTTCTTTAGGTTTGACAGGGGTTAGTTTTTA |
| | | | AAAATATATTCTTTTTTAGGCATATTTAATTTAATTTCAGTAATCAATT |
| AVPR1A | rs3803107 | 9 | AATCAATTCATTGTGTATGAGACTGTGTTTCTAGTTGCATTTTCATATTG |
| | | | CTACCAAAAACTAGACATTATTTTGTATGGAATATTAATGGAAACATGCT |
| | | | GTACTAAAATATGCAGGTCTGATTCCCAGAAATACAACAGAAGTTATATT |
| | | | TTTAAAGGAAAAATCATAACCACCCTAGCTTTATATTTTGTTGTTAGTTT |
| | | | YTTTTATTTTCATTTCTAACATAAGTAAGACTTGATTGGTTTAAAAGTCA |
| | | | CATAAAATGCGGCACTATTTCTGAACAAAGAGAGCTCATCATCAGTCTTA |
| | | | ATATTCAGAGAAAACTTCAGAGAAATTATGTTTTCATCCATTAAAATTAA |
| | | | TTTGTGCATCAGAAAATGCAGCCTTAAACAGTGTCCAGGAGATGGGATGG |
| AVPR1A | rs1495027 | 10 | CATATCAAGAAGAATGTGAGTATTTTGGAGGTCCATCCTAGTTATAAGGA |
| | | | AACTTCAAACCGTATCATGAGAGAAATGTTGAAAATAACTGTTTCTACTG |
| | | | AAGAAACAGTAAAGGCTCTAGATTTCAAATATTTTGAGAGTCATTATGTG |
| | | | TAACAGGAATTAGACTTGTTCTGAATGTTCCTAAAGAATGGAATGAGTGT |
| | | | YAAAGTTTGTAAATTTACATTTATTTGCACGATTACTTGTTTTATATGTT |
| | | | TCCCCTCCGCTGGTGTCTAAGCTTCCTGATGGCAAAAGTTAGATTTGGTC |
| | | | ACCAATTTATCCCCAGTGCCTAACATGCATAAGAGCCACTTATATAATGG |
| | | | TTAACAGACTGAGAGAAATTTTTTTTATTCTCTAGTGTAGGAGTTAGGGT |
| | | | ACAAAATAAGTTGTTATAACAAA |

The Sequences given in TABLE 1C (SEQ ID NO:1-10) above and in TABLE 1D (SEQ ID NO:11-264) would be useful to a person of skill in the art in the design of primers and probes or other oligonucleotides for the identification of vasopressin pathway associated gene SNP alleles and or genotypes as described herein.

TABLE 1D below shows the flanking sequences for a selection of vasopressin pathway associated gene SNPs in LD with the tagged SNPs in TABLE 1C, providing their rs designations and corresponding SEQ ID NO designations. However, where a SNP in LD is also an htSNP it only occurs in TABLE 1C above. Each SNP is at position 200 of the flanking sequence (unless otherwise indicated) and is underlined.

| | | | |
|---|---|---|---|
| | | SEQ | |
| | | ID | |
| GENE | SNP | NO: | FLANKING SEQUENCE |

| | | | |
|---|---|---|---|
| LNPEP | rs10038651 | 11 | TCTTCAGACCCTCCAATAAAACTTATTTAATCCTAAATGGGTCCTGT |
| Region | | | TAAAAATTCCTTCATTATTTTGTCATGCTTTAAGACCCAGGCAAAAC |
| | | | TCTTGGTGGGCTTTTGTTAAATTCCAGCCTTTGTATAAGGGCACTGG |
| | | | CTTTTAATATTTAACTTAACCACTCAGCCAGTACTGAAACAGTTGTT |
| | | | ATGGAGGCCTGCRTTAGTGAGATCTGCCTTGCCACACTTGTGTTACC |
| | | | CACTCTTTCCAGAGTATACTTTCTTCCCTTCTTCACCTTTTCAAATA |
| | | | CTCATCTTTTTAGGCCCTCTTCAGGTTTTCTGCATGTTTCCTTATAA |
| | | | TATCTTCAACCTCTAGTCAGAATTTGTTTCCTTCCCTTTGTTCCCAT |
| | | | TGCTTTATTTTCATTGTTAGGACAT |
| LNPEP | rs10044354 | 12 | CAGGCAAAACTCTTGGTGGGCTTTTGTTAAATTCCAGCCTTTGTATA |
| Region | | | AGGGCACTGGCTTTTAATATTTAACTTAACCACTCAGCCAGTACTGA |
| | | | AACAGTTGTTATGGAGGCCTGCGTTAGTGAGATCTGGCTTGCCACAC |
| | | | TTGTGTTACCCACTCTTTCCAGAGTATACTTTCTTCCCTTCTTCACC |
| | | | TTTTCAAATACTYATCTTTTTAGGCCCTCTTCAGGTTTTCTGCATGT |
| | | | TTCCTTATAATATCTTCAACCTCTAGTCAGAATTTGTTTCCTTCCCT |
| | | | TTGTTCCCATTGCTTTATTTTCATTGTTAGGACATGACTTACAGCCT |
| | | | GATGTAAGTTTCTGTTCATTGTATAAACCTCTGCCTTTCCCAGTTTA |
| | | | TTGCAGATCCTTTAGTAACTAGGAT |
| LNPEP | rs10058476 | 13 | TGAGGATTGGTTCCAGGATCCCCTCCCCCCTACCAAAATCCACCAAT |
| Region | | | ATTCAATCCCTGTATATTTGCATATAACCAGTTTACACGAATCATCC |
| | | | CATTTACTTTAAATTATCTTTAGATTACTTACAAAACATAATACAAT |
| | | | GTAAATACTATGTAAATTATACTGTATTATATTATTATTTTTGATTT |
| | | | TTTCAATTTTTTWAAATCTGCCATTCAGTCTATAGATCTGGAACCTG |
| | | | TAGATACAGACTAACTGTATTTGGATAATTTCATAATTTTAATGAGA |
| | | | GAAAGGGGAGAGGGGAAAGCCTGGTTTACTGCCCATGATGAAGTAGT |
| | | | AATACAGTAAATTTAGTTGAGACATCAGCCAACCTTTTTTGAATACC |
| | | | TACTAAGTACCTGGCTGAGAGAGTT |
| LNPEP | rs10061936 | 14 | TCCATTTTTCTCTTTTTGAATTTTTTCCTTTTCACATTACTTTAGTA |
| Region | | | ATTTGTTCTTCATCTCTTATTTTTATCACCTAGACAGAAAATATAGC |
| | | | AAAGCATAAATCATTTTTCAGGTCACCATGCTTCATTCTTCTTTTAT |
| | | | TGGGGAAGGGGCAGTGGTGATCCGGGAAGAAGCATAGTGTAAACATT |
| | | | TTAATACAAATTYCTCTTTTTTTTTTTTTTTGAGATGGAGTCTTGCT |
| | | | CTGTCTCCCAGGCTGGAGTGCAGTGGCACGATCTCGGCTCACTGCAA |
| | | | CCTCTACCTCCCGGGTTCCAGTGATTCTCCTGCCTCAGCCTCCCGAG |
| | | | TAGCTGGGATTACAGGCATGCACCACCATGCCCGGCTAATTTTTATA |
| | | | TTTTTAGTGGAGACAGGGTTACACC |
| LNPEP | rs10069361 | 15 | ATATTCAAATCCTGGCTCTTTATTCACTAGCTCTCTGATTCTTAAGG |
| Region | | | ATATTACCAGAATATCTTAATATCTTTAGTTAAAAACCTAAAATGTA |
| | | | CATTCAAAACTTAAAACTTTTTTGAAATTAGCAGTGGTCTAAGATAA |
| | | | GTGGTGGTTTGAGCATATTCCAGCCTTAGTGAGGTTTTGAAAAGCTG |
| | | | GGAACTAATGGTRTTTCTTGGATCCTAATTCTTTACTAAGGGCTTGA |
| | | | GGCCATTATAGGAGGATTCTTTCCATTTCATATTTATTAACAATTTT |
| | | | GAATTTGCAACACTTTCATGGAAGTGTTGCCTAAAGCATGGGTCCCC |
| | | | AATTTGCATGTCAAGGACCATGCTAGGAACTGGGCTTCACAGCAGGA |
| | | | GGTGAGCAGTGGGCAAGTGAGCGTT |
| LNPEP | rs10071975 | 16 | ATTTGGGTCCACTAATTAGAGTTCTTCATCTTTCTTTTTACATGTGG |
| Region | | | ATATGTGTTGCCTTTCATTCATCTGTAATTTCCAGGTCCTTAAAAAA |
| | | | AAAAAGTAGATTGAGAATGCAGGCATTTTGAAGACTGGGTGCAAAAA |
| | | | TCCTAGAATTCTGCCTCCCAACCCCAACCCCCAACCCCAAGGTATTA |
| | | | GGTTTTTCTTGCSCATACCTAATTTGGCAGCAGTGTTATTTTGAGGA |
| | | | CTCATTTTTGTAGGATCTTTCTGATACATAACTCAGTTTTCATAAAAA |
| | | | ACAATTTTTATATTTTTCATTTAATGACACAATATTTAATTATTATA |
| | | | AAACCATAATTACAAGTTTAACTAACATAAATCAGCTTGAGAACAAA |
| | | | CAACTAATTCTTAGAGTAGAGTGCC |
| LNPEP | rs1019503 | 17 | TGCTCTCTGAAATGCCCTGCTAAATGCTTCTCTTAATTATTTGAATA |
| Region | | | AGGTAGTTTGGAATAAAGAAAGAAAAGATCACTCTACATACAGATAG |
| | | | TAAACTTAATTTGTGATCCTATATATGAGACAGTATAAAAATACAGA |
| | | | TAAGTTTTAGAAAGACTCAAAACAATATGTAAATGACTGATGTTTGC |
| | | | ATTATTAAGGAARACTTGGGATGTTGGGTCAAGAGGGGAAAGTGTTA |
| | | | GTCAATCCACTTTGGAGCAATATCATGAAGGTCAATTATAATTCCAT |
| | | | ATACCTTTCTTTGATGCCACAGTCAGAGATAGAATACAGTTTGGGTG |
| | | | GCCATGGATGTGCCCCAATACAGTACACATTTTTTGGTTAAATTTGT |
| | | | TTTCAGATCATTTCATGGAATCTTT |
| LNPEP | rs10434708 | 18 | GGAAAGAGATGGGGAGAAAAAGAAGGAACACAGTGACTGCTCTGTTC |
| Region | | | AAAATAGGGGTCCACATGTCCAAGATGCTGTGGCTCCCTGTGGCGGA |
| | | | CATCAACGCTCTCATCCATTATGCTCCTCTTCTGTGGGAGGGAAACA |
| | | | CACCTCCCATCGTGCTGCTCTTCTATGCCCAGCAGCATTGATTAGAG |
| | | | AATGGATTTTCCWTTAAAAAATACATACACACACACACACACACACA |
| | | | CACACACACACGCATTGCATATTAGAATTAGAGGGATTTCTGGAGGA |
| | | | ATCACCATACCTTATTTGTACAAGGTCAGCAATCTTTTATAAAAGTT |
| | | | GTCAAAAGTTTATGTAGAGAGAGAACTGAAAACTATGCTTCCATCCG |
| | | | TTATCTGTGTTGGGCACTGAGGTTG |
| LNPEP | rs10434709 | 19 | CATATTAGAATTAGAGGGATTTCTGGAGGAATCACCATACCTTATTT |
| Region | | | GTACAAGGTCAGCAATCTTTTATAAAAGTTGTCAAAAGTTTATGTAG |
| | | | AGAGAGAACTGAAAACTATGCTTCCATCCGTTATCTGTGTTGGGCAC |
| | | | TGAGGTTGGATGGTAAGACTGTGGAACAGATTTTTAAAAAAATTGCAG |
| | | | GAAACAGATCATYTGGTTGTGGTAGTAGGTCTTTACATGAGATGATA |
| | | | CTCATAGTCTATCTTGCTTTTAATTTTCTATCTTAAAAAATAAAAAA |
| | | | CGTTATTTTTAGAAGGTTGTAGAGAAGCGATCCCCAACCTTTTTGAC |
| | | | ACTAGGGACCAGATTTGTGGAAACAATTTTTCCACGAAGATTGGGTG |
| | | | GATGGTTTTTGGATGAAACTGTTCC |
| LNPEP | rs1046395 | 20 | TTGTATGCTGTGCTTCATTCATGGGGCTGTGAACTACTGATTATATT |
| Region | | | CTCCCTATTCCTAATGTAGAATGCTTTATTCTACTGCCATCTTTCTG |
| | | | TCTGCACTGTTTAATTAGGCTTACTGATAACAACTTTAATTCTGAAT |
| | | | TTTCTTTCTCATTCAGGTTCTATTTGTAATTACTAAGACTTAAAGAA |
| | | | TAGTCTGGTAARTTACTCGAAGAATTAAGGAAGGTTTGAGCTAAAA |
| | | | TGAACTAGAGACCATCTAGTACTTTAGTGTAAAATATGTTTAATACA |
| | | | AGTCGTTAAGTCCTTGTAAGTGACTATTCCAATGTTCATTCTTTGTT |
| | | | TTTGGAAGAATGCTTGGAGTTACCATGTTTTTAAATGTGAAATTTCA |
| | | | TCTAAATTAAAAAAAAAATCTCTGT |
| LNPEP | rs10476696 | 21 | TCCCAAAGTGCTGGGATTTCAGGCGTGAGCCACCTGGCCTGGACTGT |
| Region | | | AATTGAGGATTTTTCTGTGTCATATTCTCAACTGTTGTTGGTGTGCT |
| | | | ACAGAAGAGGAGGAATTTTTTTTAATCTCTGAGGCGAGTAAAGGA |
| | | | AACCAGAATACTACAGGACACCTAATTTTTTCAATCTTCATGAAAAT |
| | | | GCAAGCTGTGAAKTTGAGGTTTGGTATCGTGAAGCCAGAGTCTGTAC |
| | | | AGATAATTCGCAGCAATTAATGACCACCCTTCTTAATAATCTTCCAT |
| | | | CAGAAACCTTTTTAAGACCTCAGTGGCCAGTTGCAGCCTACCTTTGT |
| | | | GGCTTCATCTCCAGCCACACTGGACAGCCACCCCCAGTTTCTGCACA |
| | | | TGCACTGCTCTCTTGTGTTCCCGGA |
| LNPEP | rs10537702 | 22 | TGAGAGTTCAACCAAGTAACATTGCCCCACTAAACACAATGTTTAAA |
| Region | | | CACAGTGGTATCCAAAATGGGATGAGGAAGTGTGCAAGAAGTGCAAT |
| | | | ACATTAGAGTGTCTATTATTTCTTATCTTATTTTAAATTTTATATTG |
| | | | TTATAAATTTATAAACATAAATGATATATAGTATAAAAAGTTAAATA |
| | | | AATACATTATTTATTT/- |
| | | | TTTCATGCTTTTAATTTTTTTACCATACCTTAACATATGCATATAAT |
| | | | TTTTTTTAATTAATTTATTTTTTTTGAGACGGAGTTTCATTCTCGTT |
| | | | GCCCAGGCTGGAGTGCAATGGCGCCATGTTGGCTCACTGCAACCTCC |
| | | | ACCTCCCGGGTTCAAGTGACTCTCCTGCCTCAGCCTCCTGAGTAGCT |
| | | | GGGATTACAGGC |
| LNPEP | rs10546363 | 23 | GTGTGAGCCACCGCGCCCAGCCCATATAATTTATAAATAAAAATATG |
| Region | | | TATATTGGGAAGTTCTTGCTCAAAAAATCTTTACTGACTGGAGTATG |
| | | | TAGTAACAAAAAAAGTAGGGAACACTGCTTTAAACAGAAACATAAAA |
| | | | TTAAACATAAACATTGCTGAATAACTAACCATATTTCCCAAAGAAGC |
| | | | TGTATCTACATTTT/- |
| | | | TCATTTTATAGTAAAATTTGATAAGTTTCACAGCTTTAGAATTGTAC |
| | | | TGGATGAATGTTATTATGGTAATTCACCGTATCTATTGTAATACACA |
| | | | AGCTTATCACATAGTTATTAATATACATTAAAAATATAATACATGAT |
| | | | ATAATAAACATAAGGTCAGTATTTCTATGACTTTCTATGGTGTTTCT |
| | | | TTTTATTTTCAG |
| LNPEP | rs1056893 | 24 | GGACTAAATTTAGCCTCTCTGTTAACCATCTCATATTTTCTGCAGCG |
| Region | | | TTACCTTCTTCAGTATTTTAAGCCAGTGATTGACAGGCAAAGCTGGA |
| | | | GTGACAAGGGCTCAGTCTGGGACAGGATGCTCCGCTCGGCTCTCTTG |
| | | | AAGCTGGCCTGTGACCTGAACCATGCTCCTTGCATCCAGAAAGCTGC |
| | | | TGAACTCTTCTCYCAGTGGATGGAATCCAGTGGAAAATTAAAGTAGA |
| | | | TGTAGACTTCTGTCCTACCCTTTGTTCTTTTCTCTTTGATGTAAAAG |
| | | | TCTTTGATCAAGCAAGACATTAGGTCTAAAACCTTTTAGTGAGGATA |
| | | | GAAAAAAAAACATGCTGGGCATTACAAACCCTGTTTCATGCTCTCAC |
| | | | ATTGTAAGTGCTATGTATGGAGACT |
| LNPEP | rs10707238 | 25 | CTTCAGAAGATTGCTGCCCACTTGTAAAGTAATCTGAAGACTGTCAG |
| Region | | | AAAAGGAATAGTGCTTAACTGTTTCTAGAAGCTACAGACTTATAAT |
| | | | TTTCTGTTCTGTAACTATAACCAGGCTCTTCTGAATCTTAGAATCTT |
| | | | ATTGTTGAAGCTTTGGTCCGTCTAGAGATTTTAATCTTAGAGACATA |
| | | | CACTAGATGTGCA/- |
| | | | GTATTAGGCATATAGACTAAATAAATAAAACATAAAAGCACATAAAA |
| | | | GAATAGTAATATTTAAATGCATAATACAGATCAAATATAGGTAAAG |
| | | | GGTAAATATGTCAATTATATTTATGCATCCTTTTTATTTGATTTATA |
| | | | TATTTTTTAAATCTTAGACCTTTATTGTTTCTAGTGGCCCACTGAAG |
| | | | TAAGTCAGGGGC |
| LNPEP | rs11135482 | 26 | TGTAGAATTTAGTAGCAAAAACATTTGCCATCAAAGTAGACTAGATA |
| Region | | | ATTTATGGTAATGCTTCAAGCTATTTTCTCTTGCCAAAGCAAATCGT |
| | | | AATCTTATCCAACATGTCAAACATGCTTAATAAGCTGCAGTCAGCAT |
| | | | CATCACAAGCCTGACTCCCAGAAAGGGCTCAGGGATAGAGGTGGGGA |
| | | | AGAGCCTGTCTARGAGTTGTGACTAGCTTGAAGAAAATGTTTTCAGA |
| | | | TTATTGGATCTGTATCCATTCAGTATTTGGGGGCATTGTACCATGGT |
| | | | GAAGACCATCTCTGAGACAAGCTGCCCAGACCAAATGAAGATAGAAT |
| | | | TCAGTCATTACCCAGTGATCTTGATAGATGCAGCTGACGAGACTGCA |
| | | | GGCTGAAAAGTTTCTGCTTCCTCAG |
| LNPEP | rs11135483 | 27 | ATCATCACAAGCCTGACTCCCAGAAAGGGCTCAGGGATAGAGGTGCG |
| Region | | | GAAGAGCCTGTCTAGGAGTTGTGACTAGCTTGAAGAAAATGTTTTCA |
| | | | GATTATTGGATCTGTATCCATTCAGTATTTGGGGGCATTGTACCATG |
| | | | GTGAACACCATCTCTGAGACAAGCTGCCCAGACCAAATGAAGATAGA |
| | | | ATTCAGTCATTASCCAGTGATCTTGATAGATGCAGCTGACGAGACTG |
| | | | CAGGCTGAAAAGTTTCTGCTTCCTCAGGAGATGGACAGAAGCTTAAA |
| | | | TTACTAATGACCTCCTTGGCCTGACTGCTTTCATTGCTGAATCAATG |
| | | | AAGCAAAGATAAAATAAGACCATGACTCAAGCTGTCACGCAGCAAGT |
| | | | GAGAGAATGAGCATCATCTTTGGAG |
| LNPEP | rs11135484 | 28 | CAATGAAGCAAAGATAAAATAAGACCATGACTCAAGCTGTCACGCAG |
| Region | | | CAAGTGAGAGAATGAGCATCATCTTTGGAGTCACACGGTCACATCCA |
| | | | CATCTTGGTCCTACCGTGGAACTAGCCATGTGATCTCCAACAATTCT |
| | | | GTGAACATTTCAGAGTCTCTGTTTCCTCACCTGAGAAACAACACCAA |
| | | | CCTCACACCCACRTAACAGGATTAAAAGATAATGTGCAGCCTCTAGT |
| | | | TCAGTTTCACTTCCTGTTTTCTTTTTCCACAGGGGTGTACTTCTTGT |
| | | | ACAACAAATAAAGGGAAAGGGGCCATTATCTGGTATTTTACTTAAAA |
| | | | GCACAGAAGTTGAATTGATGCCAGTGTTGGAAATTATTGCATTTTAA |
| | | | GAAAATAGAAATATGTAATATTTTT |
| LNPEP | rs11135485 | 29 | TTCTTGCTGTGTCCTCACATGGTCTTTGTTCTGTGCATATGTGGAGA |
| Region | | | GAGCGAGCTTTGCTGTTTCTTTCTATCAAGGACACCAATCCTATTGG |
| | | | ATTACGGCTCTACCCTTATGACCGAATTTAACCTTAATTACCATCTT |
| | | | AAAAGCCCTGTCTCCAAATGCAATCACAATGGGGGTTAGTGCTTTTT |
| | | | TTCTTTTTTTGGSGGGGGGCGCGGGGGACAGAGTCTTGCTCTGCCAC |
| | | | CCAGGCTGGAGTGCAGTGGCGCGATCTCAGCTCACTGCAAGCTCCGC |
| | | | CTCCCGGGTTCACGCCATTCTCCTGCCTCAGCCTCCCAAGTAGCTGG |
| | | | GACCACAGGCGCCCACACCACGCCTGGCTAATTTTTTGTATTTTTTA |
| | | | GTAGAGACGGGGTTTCACTGTGTTA |
| LNPEP | rs11311774 | 30 | ATATTTTATTTTTAAAGTAAATTTATACAACTTTTGTAAGTTCTAAA |
| Region | | | TTAATTTGAATATAGTTTGTTTTAACTATAGTATCAGTATATCTTTA |
| | | | AGATATTGTAATCAGGTTATAGATAATTAATATGACACTTCAGCCAA |
| | | | TTATTTAAAAAATTCCTGAGGCTGTAAATATCCTGTGGGTTAATTGT |
| | | | TTTCTCTCCCCCC/- |
| | | | AGTGGTTTGGCAATCTGGTAACAATGAAGTGGTGGAATGACCTATGG |
| | | | CTAAATGAAGGTTTTGCCACTTTCATGGAGTATTTCTCTTTGGAAAA |
| | | | AATATTCAAAGAGCTTTCTAGTGTAAGTACAGGGTTTCTTTGGCCTA |
| | | | CTATGAATGCTAGGAGGAAAAATAGTCAAATCACATTTTCATGTATT |
| | | | TTCTGTGCATCT |
| LNPEP | rs11414909 | 31 | CCCTTGCCTCCTCACTCCCTCGGCCTACTCCTGTTTATTCTTCAGAT |
| Region | | | CTTAGCTCAGCCATTGCTTGCTCCAGGAAACCTTTCCTTCCCTGAAG |
| | | | ACAGTTTAGATGCCCTACTTAGGTTTTTTAATAACATTCTCTACTTC |
| | | | TCCACTCATAATATACTGTAAGTACTGTTCACTCATATCTATCCTCA |
| | | | TATTAGGTATTT C/- |
| | | | CCCCATTGACGGTAGTGATCATGGCTATATGAATCACTGTAAATCAC |
| | | | TTTTAGCACTTAGTAGGCACACAAAAACTTAATGAATTAGTAAATTT |
| | | | TAGTCCATAATGAAGTGACTCCAGTCTCACTATAAAAATTTCTAGGA |
| | | | AAAGAACATGCAAAGCCTGATAAAATGATGTTTTCTTTTTCTCTTCC |
| | | | TCTTCTTAGTAA |
| LNPEP | rs11750025 | 32 | CTTTTCTTACAGTATTTTATCAGTACCTTCCTCTTTATTGGAGCTTA |
| Region | | | GAAATAGATTTCAAATAGAATTCAGCAAAATTAAATTCTGTAGAATT |
| | | | TAGTAGCAAAAACATTTGCCATCAAAGTAGACTAGATAATTTATGGT |
| | | | AATGCTTCAAGCTATTTTCTCTTGCCAAAGCAAATCGTAATCTTATC |
| | | | CAACATGTCAAAMATGCTTAATAAGCTGCAGTCAGCATCATCACAAG |
| | | | CCTGACTCCCAGAAAGGGCTCAGGGATAGAGGTGGGGAAGAGCCTGT |
| | | | CTAGGAGTTGTGACTAGCTTGAAGAAAATGTTTTCAGATTATTGGAT |
| | | | CTGTATCCATTCAGTATTTGGGGGCATTGTACCATGGTGAAGACCAT |
| | | | CTCTGAGACAAGCTGCCCAGACCAA |
| LNPEP | rs1216565 | 33 | TAAAATTCTAAGCCTCCCAAGTGACTGAACAGACCATGTCTTGGCCA |
| Region | | | AGGGGACCCCAGGGTAACCTTGAAAACTAAATTCTCATTCATGACAG |
| | | | GATGCCAGGGTCAAACAAGCCTTATTATACCCCTTCCTCAATATTCA |
| | | | GGATTAGCCTTTCTTCCCTAAGGGCTAAACGGAAACCAGCCCTTTTG |
| | | | AAAGATTCCACCMCTAATATCAACCAACCACCTGATATTGCCTCTAG |
| | | | TTTTTTGCCTGATAAGAGATCACCACATGGAGTGGTTCTGGCCCATC |
| | | | TCCAGAGAATGCACAGTAAGAGTTTTCATGTCCTCTGCTTCACCTTT |
| | | | TGATGTCAGAGGACTGAAAACTCCACCCTCGGATCATGTTAACACTG |
| | | | CCATTTTTTGTATATGGGACCCATG |
| LNPEP | rs1216566 | 34 | GTGCTGGGATTATAGACATGAACCACCACGCCTGGCTATCTTTTCAT |
| Region | | | TTCTTGATACTATCCTTTGAAGCATACTTTGTTGATACTTATCTTCA |
| | | | ACCTTATTTCCATTACAATGAAGTTGTTATGAGTTGAATAGTGTCCT |
| | | | CCAAAATTTATCTGTTAAATTTATAATCCCCCATATTTCAGAATGTG |
| | | | ACCTTATTTGAARTAGGGTTGTTGCAGATGTATTAGTTAAGATAAGG |
| | | | TCATACTGGAGTAGGGTGGGCTTCCAATTCAATATGACTAGTGTCCT |
| | | | TATTAAAAGAGGAAGTTTGGACACAGGTATGCACACAGGAAGAATGT |
| | | | CATGTGAACACTGGAGTTACTTGCCACAAGCCTAGGGACTATTAGAA |
| | | | CCTAGGAGATAGGCCTAGAACAGAT |
| LNPEP | rs1216567 | 35 | TTGCTCTTTTGCCCAGGCTGGAGTGCAGTGGCATGATCTCTGCTCAC |
| Region | | | TGCAAACTCTGCTTCCCAGGTTCAAGTGATTCTCATGCCTCAGCCTA |
| | | | TTGAGTAGCTGGGATTACAGACACAGACCACCATACACAGCTAATTT |
| | | | CTTGTATTTTGTATTTTTAGCTAAGCTGGTCTCAAACTTCTGGCCTC |
| | | | AAGTGATCCGCCYACCTCAGCCTCTCAAAGTGCTGGGATTATAGACA |
| | | | TGAACCACCACGCCTGGCTATCTTTTCATTTCTTGATACTATCCTTT |
| | | | GAAGCATACTTTGTTGATACTTATCTTCAACCTTATTTCCATTACAA |
| | | | TGAAGTTGTTATGAGTTGAATAGTGTCCTCCAAAATTTATCTGTTAA |
| | | | ATTTATAATCCCCCATATTTCAGAA |
| LNPEP | rs1216568 | 36 | TCCCAAAGTGCTGGAATTACAGTCCTTTGCCTACTTTTAATTGGATT |
| Region | | | ATTTATCTTTTATCATTAAATTTAAAAATTCTTTATATATGCTAGAT |
| | | | ACAAGTCCCTTGTGAAGTCCCTTGGTTTGTAAGTATTTTCTCCTATT |
| | | | CTGTGAACTGTCTTTTCATTTCTTTCTTTCTTTCTTTCTTTGAGACA |
| | | | GAGTCTTGCTCTKTTGCCCAGGCTGGAGTGCAGTGGCATGATCTCTG |
| | | | CTCACTGCAAACTCTGCTTCCCAGGTTCAAGTGATTCTCATGCCTCA |
| | | | GCCTATTGAGTAGCTGGGATTACAGACACAGACCACCATACACAGCT |
| | | | AATTTCTTGTATTTTGTATTTTTAGCTAAGCTGGTCTCAAACTTCTG |
| | | | GCCTCAAGTGATCCGCCCACCTCAG |
| LNPEP | rs1216569 | 37 | AGCCTGGGCAACCTGGTGAAACCCCGTCTCTATGAAAAATAAAAAAA |
| Region | | | TTAGCCAGGCATGGTGATGCATGTCTGTAGTCCCAGCTACTTGTGGG |
| | | | GCTGAGGCGGGAGGTTCGCTTGAGCCTGGGAGATCGAGGCTGCAGCG |
| | | | AGCTGAGACTGCACCAGTGCACTCCAGCCTGAGCAACAGAGTAAGAC |
| | | | CCTGTCTTGAAAMAAACAAACAAACAAACAAAAATGGTAGATGAATG |
| | | | TTCATAGCTGCATTATTCACAATAGCCAAAAAGTATAAACAACACAA |
| | | | ACGTCCATCAACTGATGAATGGATAAATAGAATGTGAAACATTTATA |
| | | | TGTATAATAGAATATTATTCAACAATAAAAAGAAAGTACTGACATGT |
| | | | TAAAACATAGATGAACCTTTTAAAA |
| LNPEP | rs1216570 | 38 | TTGAATGAAATCTGTACCTTTTTAAATAGTAGCAACCAGATGGGGGA |
| Region | | | AAACAAACTTGCTTGAGCAATGGTGAATGGAGATACTCACAGTATAA |
| | | | TTTGCTTTTTTTTTTTTTTTTTTTTTGAGACGGAGTCTCGCTCTGTC |
| | | | GCCCAGGGCTGCAGTGGCGTGATCTCGGCTCACTGCAACCTCTGCCT |
| | | | CCCAGGTTCAAGYGATTCTCCTGCCTCAGCCTCCCAAGTAGCTGGGA |
| | | | CTACAGGCGCGTGCCACCACGCCCGACTAATTTTTTGTATTTTTAGT |
| | | | AGAGATGGGGTTTCACCGTGTTAGCCAAAATGGTCTCAACCTCCTGA |
| | | | CCTCATGATCTGTCCACCTGGGCCTCCCAAAGTGCTGGGATTACAGG |
| | | | CTTGAGCCACCATGCCCAGCCATTA |
| LNPEP | rs12189125 | 39 | AACCATAGCAAACGCCCATTTGCCTCCGAACCATCTCTGCCACCAGC |
| Region | | | CTTCTAGTAGCCCAGACGTATTTCCCCATAGTCTCACAGCCTCACGC |
| | | | CTCTGCCAGTAACCCCTCCACACACTTGACTAAATGGTTTTGCTGCT |
| | | | GAGTTTGGTCAGAAGACCACAATAATACCCCAGCTCTCAGCCCCTAC |
| | | | CATAAGACAGCAYCTCCTCTGCTGGGAGTGGATATCCAGAGAACACT |
| | | | GGTTGAATCAGCTTCCTAAAATGGAGACGGTTGTTGGGGAAAATTA |
| | | | ATTTGCTGGATAGAGTTCTTAAAAATTACAGCCCTGTATATACTTTG |
| | | | ACTTTTCTTACAGTATTTTATCAGTACCTTCCTCTTTATTGGAGCTT |
| | | | AGAAATAGATTTCAAATAGAATTCA |
| LNPEP | rs1230358 | 40 | TGCTAAATCTGGGTACTGGAAAGGATAAAGAGAGGGCAGAGCAAAGG |
| Region | | | CCAGAGGTTTCATCTTTGTGGAAGGTCTGTATTCAGAGCAGAGAGGA |
| | | | AGTTGAAGCCCAACTCAAACAGGCAGATAAAGAGAGATCAAAGAGAT |
| | | | GAGCATGAGATACAGTCCCCTCGTGCCCAAGGAGACAGGGTGGTTAC |
| | | | AGACATGGAAAAKCTGAGAATAATCACCTCTGATAAAGATCACAGAA |
| | | | GCTGCCCGGGAGGTGTTTGGTAAGCTTGGAGTTACGTTTGTGGGGTG |
| | | | GATGGGCAGAAGTCAGATTTCATAGCACTGAGGATGCAGCACAAGGA |
| | | | GAAGTTCAAGATCAATTCCTAAGACAACAACTTGGCACTAAAAAACA |
| | | | TAAACTATGTTCTGAAGGCTTTACC |
| LNPEP | rs1230360 | 41 | TTGAGAGAGAGCCTCGCTCTGTCGCCCAGGCTGGAGTGCAGCAGCAC |
| Region | | | GATCTCGGCTCACTGCAACTTCCACCTCCCTGGTTCAAGCGATTCTC |
| | | | GTGCCTCAGCCTCCCGAGTAGCTAGGACTACGGGCATGTGCCACCAT |
| | | | GCCCGGCTAATTTTTGTATTTTTAGTAGAGGTAGGGTTTCACCATGT |
| | | | TGGTGAGGCTGGYCTCGAATTCCTGACCTCAGGTGATCTGCCCACCT |
| | | | TGGCCTCCCAAAATGCTGGCATTACAGACCTGAGTCACTGTGCCCGG |
| | | | TCCTGTTTCTTTATCTGAACACTAAGGACTTGTACTAGCTGGCCTTT |
| | | | ACAACCCTTACTAGTTCTAAGTTAAAGACTGTGTGAGTAAAGCTTT |
| | | | TCTCTCTACTCTTATCAATCAAGTA |
| LNPEP | rs1230363 | 42 | GGGCAACATGGTGACACATTGTCTTTCAAAAAAAATAAAATATGGCC |
| Region | | | AGGCGCAGTGACCCACGCCTGTGATCTCAGCACTTTGGGAGGCTGAG |
| | | | GCAAGTGGATCACCTGAGGTCAGGAGTTCGAGACTAGCCAGGCCAAC |
| | | | ATGGTGAAACCCCGTCTCTACTAAAAATACAAAAATTAGCTGGGTGT |
| | | | GGTGGCACATACYTGTAATCCCAGCTACTCGGGAGGCTGAGGGAGAA |
| | | | GAATCACTTGAACCCCAGAGGCAGAGGTTGCAGTGAGCCAAGATAGT |
| | | | GCCACTGCATTCCAACCTGGACAACAGCGAGATTCCGTCTCAAAAAC |
| | | | ATAAATAAATGAATAAAAATAAAGTAGGCTGGTCACAGTGGCTCACG |
| | | | CTTGTAATCCCAACAGTTTGGGAGG |
| LNPEP | rs1230364 | 43 | CAGCACTTTGGGAGGCTGAGGCAAGTGGATCACCTGAGGTCAGGAGT |
| Region | | | TCGAGACTAGCCAGGCCAACATGGTGAAACCCCGTCTCTACTAAAAA |
| | | | TACAAAAATTAGCTGGGTGTGGTGGCACATACCTGTAATCCCAGCTA |
| | | | CTCGGGAGGCTGAGGGAGAAGAATCACTTGAACCCCAGAGGCAGAGG |
| | | | TTGCAGTGAGCCRAGATAGTGCCACTGCATTCCAACCTGGACAACAG |
| | | | CGAGATTCCGTCTCAAAAACATAAATAAATGAATAAAAATAAAGTAG |
| | | | GCTGGTCACAGTGGCTCACGCTTGTAATCCCAACAGTTTGGGAGGAT |
| | | | TGCTTGAGTTTAGGAGTTTGAGACCAGCCTGGGTAACAGGGAGACCC |
| | | | CCATCTCTACAAAAAAGGTAGCCGA |
| LNPEP | rs1230365 | 44 | CCGTCTCTACTAAAAATACAAAAATTAGCTGGGTGTGGTGGCACATA |
| Region | | | CCTGTAATCCCAGCTACTCGGGAGGCTGAGGGAGAAGAATCACTTCA |
| | | | ACCCCAGAGGCAGAGGTTGCAGTGAGCCAAGATAGTGCCACTGCATT |
| | | | CCAACCTGGACAACAGCGAGATTCCGTCTCAAAAACATAAATAAATG |
| | | | AATAAAAATAAARTAGGCTGGTCACAGTGGCTCACGCTTGTAATCCC |
| | | | AACAGTTTGGGAGGATTGCTTGAGTTTAGGAGTTTGAGACCAGCCTG |
| | | | GGTAACAGGGAGACCCCCATCTCTACAAAAAAGGTAGCCGAGTGTGG |
| | | | CGGTGTGTGTCTGTAGTCCCAGCTACTCTGGAGGCTGAGGTGGGAGG |
| | | | ATCACTTGAGCCCAGGAAGTTGAGG |
| LNPEP | rs1230381 | 45 | AGTATTCTATAGTTTGCCCAACCAGTTTTACGTCCAAGGAAAATTAG |
| Region | | | CCAATGCATAAAATATACAAACTATGAAAGGCAAGGATCAGGAAACC |
| | | | AGAGACTTTGCCACCAAATCTCAGATTATTAGAAACTAGGTGTCAGG |
| | | | GTTTATCAAGAAGGCCAGGAAGGCCTTTTGGGTTAAGCCTTACATTC |
| | | | ATGAAGAACCTCRAGGGTAGATTTTTGAGAGCATTCCAAATGAATGG |
| | | | TCTCTGGTCAAATGAATGAATGGTCAAATGAATAAATCTGCCCTCAC |
| | | | AGAGATACAAAAGGAAAAGGAATATAATTCATACCATTTGGTTTAAG |
| | | | CCTTACATTCATGAAGTACCTCAAGGGTAGATTTTTGAGATCATTCC |
| | | | AAATGAAGTCGAATCTGCCCTCACA |
| LNPEP | rs1230382 | 46 | ATCAAGAAGGCCAGGAAGGCCTTTTGGGTTAAGCCTTACATTCATGA |
| Region | | | AGAACCTCAAGGGTAGATTTTTGAGAGCATTCCAAATGAATGGTCTC |
| | | | TGGTCAAATGAATGAATGGTCAAATGAATAAATCTGCCCTCACAGAG |
| | | | ATACAAAAGGAAAAGGAATATAATTCATACCATTTGGTTTAAGCCTT |
| | | | ACATTCATGAAGWACCTCAAGGGTAGATTTTTGAGATCATTCCAAAT |
| | | | GAAGTCGAATCTGCCCTCACAGAGACACAAGAAAGGAATATAATTCA |
| | | | TACACTATTGCATTTTTAATAAATCTTTTGAAATTTGCAGAATTAGA |
| | | | TTGTATTGTGTATTTTCGGTTAAATGATAATTGAATGTAAATATTTA |
| | | | GATGCAGCACCATATTTTATAACCC |
| LNPEP | rs12516666 | 47 | CATAATGAAATACTTCAAGTGAAATTTGATGGGTTGATGATCCTGGG |
| Region | | | CACATACCTAACTCTCTGAAGTTCAGTGTCCCCATCTATAAAATTAA |
| | | | GTTAATAATAGTTCTGTTTCATAAAGCTGTTCTGAGGATTATGGATA |
| | | | GGGAAAGTGTGCGGATCACATAGTAAGCACTCAATAAGTATTAGTTA |
| | | | TTAATGATGATGWCAACGGCCACTACAACTACAAGAAATACTACTAT |
| | | | TTCTTGCAAAATAACTTATCTAAGGGCCATCTATCAACACTGTATTA |
| | | | CATACAAATGTGGAATTGTAAAACTAGGTCTATAGATATTGGAGACT |
| | | | ATTCCCTCTATTTCATTTCTGAAAACTCTCAGTAATGCAGTAAATTA |
| | | | TTAAAGTCACCAAAATTGTCTTTCA |
| LNPEP | rs12716486 | 48 | TTCTTTTTTCCCTCTCATTTAGTTCTTTTTTAGTCTTGATTTCCCCA |
| Region | | | CGGAGAGTCTCATCTATTCACATATTCTCATTTTTTCCTTTTTAAAA |
| | | | TACATCTTCCTGCTTAATGATGGGGATACAATTGAAAAATAATAAAA |
| | | | CACGTCTTCTTCAGGGATTCTTTTTTATTTATAATGGCTACTCTAAA |
| | | | GACTCACTAAATRCAATGCAATATCTGGACCACCTTAAGATTGCTTT |
| | | | CTAATGATTTTGTTTACTTAGGGTTCACATTTTCTTGTTTCATTAAA |
| | | | TGTCTAGTAATTTTTTATTACATATTGAATAGTGTCAATCGCACATG |
| | | | GTAGAGATGCTGAATTAAAAAAAACTCTGTAAAATGTTGATTTTTCT |
| | | | CTCTCTGTCTCTGTAGACAGCTTAG |
| LNPEP | rs13167902 | 49 | CAACAATTCTGTGAACATTTCAGAGTCTCTGTTTCCTCACCTGAGAA |
| Region | | | ACAACACCAACCTCACACCCACATAACAGGATTAAAAGATAATGTGC |
| | | | AGCCTCTAGTTCAGTTTCACTTCCTGTTTTCTTTTTCCACAGGGGTG |
| | | | TACTTCTTGTACAACAAATAAAGGGAAAGGGGCCATTATCTGGTATT |
| | | | TTACTTAAAAGCMCAGAAGTTGAATTGATGCCAGTGTTGGAAATTAT |
| | | | TGCATTTTAAGAAAATAGAAATATGTAATATTTTTATGCTTTCAATC |
| | | | AACAAAATGAGATTTGGCATTTTTGTGCTTTGGGGATCTCAAAAGCA |
| | | | GGGCTTTTTGTTTTCAACAGAGTGTTGGGGTAAAAGCAATGGAGGTA |
| | | | AGAGAGGCTACAGAATACTAGGAGA |
| LNPEP | rs13170029 | 50 | AGCCAGGAGTTGAGGTTGAAGTCACCATTGCAGATGCTTAAGTCAAC |
| Region | | | TATTTTAATAAATGATTACCAGTTGTTTAAAAAAAAAAAAAAGAAAA |
| | | | CTATAGAGAGCTATCTACCTTTTGGGACTACCATGGTAGCAGTCATT |
| | | | TGCTGTTCCTTTTTTTGGGAGGGACGGGAACAGGGTCTTGCTTGGCT |
| | | | GGAGTGCAGTGGYACGGCCACAGCACTGCAGCCTTGACTTCTCAGGC |
| | | | TCAAGCGATTCTCCTGCCTCAGCCTCCCGAGTAACTGGGACCACAGG |
| | | | TGCACACCACCATGCCTGGCTAATTTTTGTATTTTTTGTAGAGATGG |
| | | | AGTTTTGCCATGTTGGCCAGGCTGGTCTCGAACTCCTGGGCTCCAAT |
| | | | GTTCTGCCTGTCTTGACCTCCCCAA |
| LNPEP | rs13189819 | 51 | ATACCTTGTAGCCTACATAGTTTGTGATTTCCACTCTCTGAGTGGCT |
| Region | | | TCACTTCATCAGGGGTCAAGGTGAGACTGAGTTCTAACGTTCTACGC |
| | | | AGTGCAGAAAAGTGTCCTGAGAGCAATGAACTTTTGTTTTCTCATGT |
| | | | TTTTCATTGTTATCAAAGTATTATGTTTATATTACAAGAAGAGATAG |
| | | | ATAAAAAACTAARTTAAAAATTATCCATAGTCCTGTCACCAAGATAC |
| | | | AACTACTGATAATATTAATGTAAGCCTTCCAAATATTTTCTATATGT |
| | | | ATGTCAGCATATATGGGTGTACATAGTAACAGTATTTACTTACTATA |
| | | | TATGTAAAGGTAATTTTCAAAGTATATATATATATATATATATATAT |
| | | | ATACACACACACACACACACACACA |
| LNPEP | rs13358339 | 52 | ATTCAGCCAACTACTTTTAAAATTTATCTTTTTTTTTTTTTTTTTTT |
| Region | | | TTTTGAGACCAAGTCTCACTCTTTTGCCCAGGCTGGAGTGCAATGGT |
| | | | GTGATCTTGGCTCACCACAACCTCTGCCTCCTGGGTTCAAGTGATTC |
| | | | TCTTGCCTCAGCCTCCCGAGTAGCTGGGATTACAGGCATGTACCACC |
| | | | ACACCTGGCTAAYTTTTGTTTTTTAGTAGAGATGGGGTTTCACCATG |
| | | | TTGGCCAGGCTGGTCTCGAACTCCTGACCTCAGGTGATCCACCTGCC |
| | | | TTGGCCTCCCAAAGTGCTGAGATTACAGGCGTGAGCCACCGTGCCTG |
| | | | GCCAAAATTTATCTTAATTCAGACTTTACAATTGACTTTATTAAATA |
| | | | AATATTTTTAAGTAGAAGAAATGTT |
| LNPEP | rs1363907 | 53 | AAATCATTTAACTTCTTTAGCCACATTGTGGTCACTTGTAAGATGAG |
| Region | | | GATTTATAATTTTTGTCTTACTTTACCTATTGTTTGAAAATAAAGTG |
| | | | AACAATTATGCAGAAAAGTAGAAAATAACCTTTTAGAGGTTGGCAGA |
| | | | GAAATGCCTATACCTGTGTGTATGTAATTTGCAAGCTCTTTTGAAAA |
| | | | TTTTTGGAAGACRAAGTGGTTTTATTGTTTCTTTATTTTTGAAACTG |
| | | | CCTCGCTCTGTCAGCCAGGCTGGAGTGCAGTGGCACCATCTTGGCTC |
| | | | ATTGTAACCTCCACCTGCTGGGTTCAAGCAATCCTCCCGCCTCAGCC |
| | | | TTCCAAGTAGCTGGGACTACAGGCATGCACCATCATGTCCCACTAAT |
| | | | TTTTGTTGTTGTTGTTGTTATTTTT |
| LNPEP | rs1363908 | 54 | GGGTTCAAGCAATCCTCCCGCCTCAGCCTTCCAAGTAGCTGGGACTA |
| Region | | | CAGGCATGCACCATCATGTCCGACTAATTTTTGTTGTTGTTGTTGTT |
| | | | ATTTTTTGTAGAGTCAGGGGTTCTGGCATGTTGCCTAGGCTCGTATT |
| | | | GAACTCCTGAGCTCAATTGATCTGCCCACCTTGGCCTCCCGAAGTCC |
| | | | TGGGATTACAGGYGTGAACCACCACACTCGGCCAAGACAAAGTGTTA |
| | | | GTAATTTTTTTCTTCAATATTTTACAGGTGAAACTATTTTTTGAATC |
| | | | TCTTCAGGCTCAAGGATCACATCTGGATATTTTTCAAACTGTTCTGG |
| | | | AAACGATAACCAAAAATATAAAATGGCTGGAGAAGAATCTTCCCACT |
| | | | CTGAGGACTTCGCTAATGGTTAATA |
| LNPEP | rs1363974 | 55 | AACTTTTGCAGGTTCATGCACAGATTTAAGGGATCCTCTTTTCTGAT |
| Region | | | TCTCTCCCCTCTGGGATTTCCCCCATGCTGTATAGCCTACAGGGTCT |
| | | | ACTTCTGGTTTCTCTGGATAGAAATATGGGACTCATTGGAATTTTAC |
| | | | CTGTTGGCATTTCCACACCACTCTGTGACCAAAGCCTGCCTTCAGGG |
| | | | CAAAGTAGAGAARGGAAATGGAACAATATTAAAACAGAAACTCACCC |
| | | | CTGTGTGTTTTGCTTCAGCAAGTTTTTGACCCTATAACCTATTATAA |
| | | | AGTGAAATGAAAATCTGGACACCTGTGAAGCGGTCCGAGTGCAAAAT |
| | | | TTGTCTAGACTTTCAATTTTTTTCCCCAGTCTTTTAGAGTTGTCTCC |
| | | | TACCTAATTCAACAACAATTTTAGT |
| LNPEP | rs1363975 | 56 | GCATGAAGGAGAGCTGCCAAGTTCTGTGTCTTGATAACCTTTCCTTC |
| Region | | | CATTCCTAGTTCAATTAACCTGAAGAAAGAAAAATAATTTGTTTCTA |
| | | | AATAGTAGGTATTATGTACATGGACATTAACTCAAGCCACCAATATA |
| | | | TTAAAAGAATAGAACAGAAAGAGGCATGATAAAAGTATAATTACCAA |
| | | | TTTTTTAATGTTYCAATTAAACTTTTACTTTTTTAGAAATAATTTTT |
| | | | ATTTTGTTCCTATCAAAAACATTTACTTATTATTTCAAATAAGTTTG |
| | | | ATTAGCATCATTTACACATCTTATATGCAAGAATGTATTTTTACAAC |
| | | | AATAATTTTTCTTCAAGTTTCTGAAGATAAAACATAACCGCTTGTCT |
| | | | AGTCTCAATCATATGATTAATAACT |
| LNPEP | rs1363976 | 57 | CTAAATAGTAGGTATTATGTACATGGACATTAACTCAAGCCACCAAT |
| Region | | | ATATTAAAAGAATAGAACAGAAAGAGGCATGATAAAAGTATAATTAC |
| | | | CAATTTTTTAATGTTTCAATTAAACTTTTACTTTTTTAGAAATAATT |
| | | | TTTATTTTGTTCCTATCAAAAACATTTACTTATTATTTCAAATAAGT |
| | | | TTGATTAGCATCRTTTACACATCTTATATGCAAGAATGTATTTTTAC |
| | | | AACAATAATTTTTCTTCAAGTTTCTGAAGATAAAACATAACCGCTTG |
| | | | TCTAGTCTCAATCATATGATTAATAACTAGGGAATACCTGTTTTCAC |
| | | | TATTTGCATTTTGTCAATATATTCTTTTCTGAAAGTAAAGTTAAAGC |
| | | | CATACACATTCTGATTCAATTATCT |
| LNPEP | rs1363977 | 58 | AATTACCAATTTTTTAATGTTTCAATTAAACTTTTACTTTTTTAGAA |
| Region | | | ATAATTTTTATTTTGTTCCTATCAAAAACATTTACTTATTATTTCAA |
| | | | ATAAGTTTGATTAGCATCATTTACACATCTTATATGCAAGAATGTAT |
| | | | TTTTACAACAATAATTTTTCTTCAAGTTTCTGAAGATAAAACATAAC |
| | | | CGCTTGTCTAGTMTCAATCATATGATTAATAACTAGGGAATACCTGT |
| | | | TTTCACTATTTGCATTTTGTCAATATATTCTTTTCTGAAAGTAAAGT |
| | | | TAAAGCCATACACATTCTGATTCAATTATCTTATGCCTTTAAAACTG |
| | | | GTGGCTGAAGTTTTAGTGACTTCACTGAATTTGTGTCAGTTTACTTA |
| | | | TAACAATTTAGTTAAATTATTGAAC |
| LNPEP | rs1423357 | 59 | AACATTGTCAGTCGTGGTAGTGACGATGATGAACTTGGTTTTACTTT |
| Region | | | TTCAGTGTCTAACCTGGTCCCGTGCTAGGACCCCAGGCAGAGCTTCC |
| | | | TATGAAGTCACGTACAACAAGGCCTTTGGGCTTAAGAGAAAAAGCTC |
| | | | ACAGCTGCACAGAGGGAGGAGTTTTTATATAAAGAATACAAAATGTT |
| | | | CTGAATACCAAGWGTTTCATTCTCTCCTTATGTTTCTGACTTGAAAT |
| | | | TTGAAGTAATCATGAGACACTGCATGTCTTCCCATTTCAAAGATGCC |
| | | | ACAGAATCATAAAACTAGTATCTAGCATATAATTTCAATTTTGTCTT |
| | | | AGGGGATAAATTAGTCTAAGAATAGTATACCAAAACATTAATTGTGA |
| | | | TAATAGTGTAGTGATCAATTTATGA |
| LNPEP | rs1423566 | 60 | ATCATCACACCCAGACCAATGGGAGCATTATACATGCATTATTTTTT |
| Region | | | GTACTCAAAAGGATGAAGTATATAACTGTCTACTGTACCATGTCATT |
| | | | TGAAAACACTAGAAAATTCTACAGCAATCCTTCAAAAGTTTTCAAAT |
| | | | AAATACCCTGTCCATCTTAAACCTGAAAAGCACTTCAAATTGCTAAC |
| | | | ATTTAATTTCTTRTTGACTGTAGATATTGTCGTTTCTGTTTTCCTTG |
| | | | TAAAAGGATGCTAAATAAGGCTCCAAGGAGTGATTGCTACATTAACC |
| | | | AAAATTATGCACTGCCAAGCTCTCTCCAGCATCAACTCTGAAAGATA |
| | | | GGAAAGAATCAGAAATCCAATTTCCTACATGAAAGTTGAAGCATTGC |
| | | | TTCTTTTTTTGTTCTCTTCTGTGGG |
| LNPEP | rs1477364 | 61 | ATCCCTCCCCAGTGCAGTTCACAATAGGGTTCATGCTCCTATGGGAC |
| Region | | | TCTAATACCACCCTGATCTGACAGGAGAGGCGCCCAGGCGGTAACGC |
| | | | TCACTTGCCCACTGCTCACCTCCTGCTGTGAAGCCCAGTTCCTAGCA |
| | | | TGGTCCTTGACATGCAAATTGGGGACCCATGCTTTAGGCAACACTTC |
| | | | CATGAAAGTGTTRCAAATTCAAAATTGTTAATAAATATGAAATGGAA |
| | | | AGAATCCTCCTATAATGGCCTCAAGCCCTTACTAAACAATTAGGATC |
| | | | CAAGAAACACCATTAGTTCCCAGCTTTTCAAAACCTCACTAAGGCTG |
| | | | GAATATGCTCAAACCACCACTTATCTTAGACCACTGCTAATTTCAAA |
| | | | AAAGTTTTAAGTTTTGAATGTACAT |
| LNPEP | rs1544777 | 62 | CATGGTGTGCAGTATGGTTGTTTCCCATGGAAATATGTTGTACTTCT |
| Region | | | GAAAGCCATGGAAGCATGAAAAACAGATTGAATTATAATTTTATCTG |
| | | | ACTTTTATTGTCTTTTGATCTTTTTAAAAATCATTTCTTGCTTATGG |
| | | | AAATTTCCCATATAATTTGCTGCTTCCCATTTGCTGTGGGACAGAAA |
| | | | CATTCCTCTTTCRGGGGAAAACAATAACCCATCCTGTTGCTTAGCCA |
| | | | TACTCAATCTTAGAAATGGGCATACCAGCTTGTGGTGCTCCCTAGAG |
| | | | AGAATGAGACTCAGGGATGAGACCCACAAATACCTTGGAAGCAGATT |
| | | | TGAGGCCTGTTGGACAGAAATTCTGGGATTGCAGTGCCCAAACCCTA |
| | | | GAAGGGGAACCGTGGACTGTAGGTG |
| LNPEP | rs1559267 | 63 | TCACTCTCTCCCCACTACACACCACTGGCAGCCCCTCAACCCTGGAA |
| Region | | | AAAGGAAATTATGCACACTATTTGCCTATACAGTCTTTCACATTTAG |
| | | | GATGAAATATTGAGTTCCAAAACCTGCTCTACATTTACTTTTCTAGA |
| | | | ATAACGGATACATTTCAATCCTGGTAACTTTTTGCTGTTCAAGAATT |
| | | | AGAAGTTGAGGAWAGAAGGTTTAGGAAACTCTCAAGGCCCGGTTTAT |
| | | | GCTGTAGAAAAAAAGAATTTCTGCATAAGTAAACTGCAATTATAAAT |
| | | | TTTGCTCCAAATATGAATAATTCCTCCAAGGAGAGGTTCATAACCTC |
| | | | ATTCATCTTTGTATTCCTGGTGCCTAGCAGGGAAGAAACCTGCCATA |
| | | | AATGTTGAAATGAAAGTAGCAATAA |
| LNPEP | rs1559354 | 64 | ATGAATATGTAGATATATGAGTTGTGTAGCACACATACACATCATGG |
| Region | | | CACCTCTGCACTTAGACATGGATGTCTATGCATAGACATGGATGTGC |
| | | | AGGAGGTGAATGGCACTTCAGAGGACAGGTTCCTGTCAGCCTCTTTG |
| | | | GATTCACGTCCCAGCTCTACAACTTTCAGCCTGGGTGATCTGGAGCA |
| | | | AGTTACTAAATCRTTATGTGTTTTTATTGCTTCACCTATAAAATGGC |
| | | | ACCTGCTTCATAGAGTGGGCACAAGTATTAAATTAGATTTTATACGT |
| | | | AAGCATTCAGCACAGTGCCTGGTAAACTGTCAAAAAATGGTGGCCGT |
| | | | TTACATTTTTTCTGCATAAAAGTTTTGAAGGACTTCAGTTAATTCAG |
| | | | AACATAAAAGTGGGTCATGAAATAA |
| LNPEP | rs1559355 | 65 | TATATACTCTTTAGTACAGATATACTAAATCCCATTTATATGTAATT |
| Region | | | CACTGCTGTACTTTAGATCAAAAGTCAAGGAAAGATTAATAACAGCC |
| | | | ATCAACAATATTAATGTTGTTCTTGAAAAATGCAGTCTTAAAGAGCA |
| | | | TATGAAATATCTTTAAGACTAACGGAAAAGAAGCATGCAGCTTAGGA |
| | | | AAAAATAGAGCAKATATAAGTCCCACTACTATAAAATCATCAATGCG |
| | | | ATTTAGAAGAAAAGTCATTCCCACATTTGAAGTGCTAACGAATACTA |
| | | | ATCTTTATTAGCGCTAATTTAGTTTTTGATTGTGTTATGTATACTTG |
| | | | TTTTTAATGTACTAGAATTAACCAGTATTAACTCCAGACAATGTAAT |
| | | | TATAAGCCAAGTGACTTGGTTCATT |
| LNPEP | rs1559356 | 66 | TTTATATGTAATTCACTGCTGTACTTTAGATCAAAAGTCAAGGAAAG |
| Region | | | ATTAATAACAGCCATCAACAATATTAATGTTGTTCTTGAAAAATGCA |
| | | | GTCTTAAAGAGCATATGAAATATCTTTAAGACTAACGGAAAAGAAGC |
| | | | ATGCAGCTTAGGAAAAAATAGAGCAGATATAAGTCCCACTACTATAA |
| | | | AATCATCAATGCRATTTAGAAGAAAAGTCATTCCCACATTTGAAGTG |
| | | | CTAACGAATACTAATCTTTATTAGCGCTAATTTAGTTTTTGATTGTG |
| | | | TTATGTATACTTGTTTTTAATGTACTAGAATTAACCAGTATTAACTC |
| | | | CAGACAATGTAATTATAAGCCAAGTGACTTGGTTCATTTCAAACTTT |
| | | | TAAAAAATTATCTTTTTTTCCAGCT |
| LNPEP | rs1559357 | 67 | TTATATGTAATTCACTGCTGTACTTTAGATCAAAAGTCAAGGAAAGA |
| Region | | | TTAATAACAGCCATCAACAATATTAATGTTGTTCTTGAAAAATGCAG |
| | | | TCTTAAAGAGCATATGAAATATCTTTAAGACTAACGGAAAAGAAGCA |
| | | | TGCAGCTTAGGAAAAAATAGAGCAGATATAAGTCCCACTACTATAAA |
| | | | ATCATCAATGCGRTTTAGAAGAAAAGTCATTCCCACATTTGAAGTGC |
| | | | TAACGAATACTAATCTTTATTAGCGCTAATTTAGTTTTTGATTGTGT |
| | | | TATGTATACTTGTTTTTAATGTACTAGAATTAACCAGTATTAACTCC |
| | | | AGACAATGTAATTATAAGCCAAGTGACTTGGTTCATTTCAAACTTTT |
| | | | AAAAAATTATCTTTTTTTCCAGCTA |
| LNPEP | rs17087165 | 68 | AAGAATATGATGTTATTTCTCAAAGGTACAATCTAGCTGAAATCATA |
| Region | | | TACAAGTAAGTAGGTGTGGACTTTTACTGTTGAGCTAAGGTTTATGT |
| | | | TTATATATGTTTTATTCTTTAAGCTAAACAAACATTCAGATAACATT |
| | | | CTATGCATTTTTTGAAGCATAGGGTTAGTAATGAGGACTTAGATTTT |
| | | | TTAATTAAACAAYTCAGTAACTATATAAAAGAAAAGGAGTCCCTTA |
| | | | TGAATAAATATTAAAATTAAAAGAAATAGGCAACTATAAAAGTAAGT |
| | | | ATTTTTAATAATGGCATTGATTTTAGTAAGAAATCAATTAGGCTGGG |
| | | | CTGGAAAGAAAAACTGGCTTAATATAAAGTAGTTTTAATATGTCAAA |
| | | | TATTCTTCTTAAAATTGTGGCCCTG |
| LNPEP | rs171647 | 69 | GCCTTTCTGGGGGAAAATGCAGAGGTCAAAGAGATGATGACTACATG |
| Region | | | GACTCTCCAGAAAGGAATCCCCCTGCTGGTGGTTAAACAAGACGGGT |
| | | | GTTCACTCCGACTGCAACAGGAGCGCTTCCTCCAGGGGGTTTTCCAG |
| | | | GAAGACCCTGAATGGAGGGCCCTGCAGGAGAGGTGGCTGCTTTTCTT |
| | | | CTTTAGGTCTAGYTTACCTCATCTCAGTTTCCTCGTTATTTCCTTAG |
| | | | CTTTCTCTCAGCTCATCTGGCAACTTTGTAGGATGCTAGTTCCATAT |
| | | | AAGAATCAAAGGCCTAAAGTAGACTTGATAAGATTTAAAGAGCTTCA |
| | | | TATAACCCGGACTTCTTTGTTCAGGAGCACCATTCTTAGTGATTCCA |
| | | | TCAGCCTTGAGAACTTCAGTTCTTG |
| LNPEP | rs1820148 | 70 | TTGAGCCTCAAGAGATTCAAAAAATAGTTTCACCTGTAAAATATTGA |
| Region | | | AGAAAAAAATTACTAACACTTTGTCTTGGCCGAGTGTGGTGGTTCAC |
| | | | ACCTGTAATCCCAGCACTTCGGGAGGCCAAGGTGGGCAGATCAATTG |
| | | | AGCTCAGGAGTTCAATACGAGCCTAGGCAACATGCCAGAACCCCTGA |
| | | | CTCTACAAAAAAYAACAACAACAACAACAAAAATTAGTCGGACATGA |
| | | | TGGTGCATGCCTGTAGTCCCAGCTACTTGGAAGGCTGAGGCGGGAGG |
| | | | ATTGCTTGAACCCAGCAGGTGGAGGTTACAATGAGCCAAGATGGTGC |
| | | | CACTGCACTCCAGCCTGGCTGACAGAGCGAGGCAGTTTCAAAAATAA |
| | | | AGAAACAATAAAACCACTTCGTCTT |
| LNPEP | rs1820149 | 71 | ATGTAGCATTGTTTCCAGGTTCTCTTAAAGGTTTTCTTTTTATCTTT |
| Region | | | AGTTTTAAGCAGTTTTGACCATGATGTGCTTAAGACATTATTATTTG |
| | | | TGTGTGTGTGTGTGTGTGTGTGTGTGTGTGTGTGTGTATTTATCTTT |
| | | | TTGGGGGTTTGCTGAACATTTTGGATCTGGTAAGTGGTGTTTTTCAT |
| | | | CAAACTTAATAAWATTTTAACTATTATTTCTTCAGATATTTTCTTCT |
| | | | CCTGCATTCTCACACTCCTTCTGTGGCTCCTGTTAGATACATGTTAG |
| | | | ACTGTCTGATACTGTCCCCCAGATCCCTGATGCTGTGTTTATTTTTC |
| | | | TTCAATCCTTTGTCTCATTGTTCTTCAAATTGGTAATTTCTAATGAT |
| | | | CTGTCAAGTTTATGGACTCTTTCTT |
| LNPEP | rs187265 | 72 | TTGAAAGGGGTCAGGAAAGAGACTCCAGTCTCAACCTCCTTTTCACT |
| Region | | | GGCTTTTCCTGCCATGTATTCACCCTACTATATCCTGTATATATCCC |
| | | | TCAATTCAAGTAATTTGCAGAGAGCAGCCCTGGGATAGCCATCCCTA |
| | | | ATCCAGTTGCCTGGATTACCCTTCCCTGAGATACCAGTCCGAGTCTT |
| | | | CTGTTCCCCAAGSCTTGTTTCTGGCATCCAAGGAGATGGAAGTTTCT |
| | | | GTCCCTCTGTCTTTGGATTGTCTCCTCTCTCTTGAGTTATCATAGTC |
| | | | ACCTGTCATTTCAGCTCGCCTTTCTGGGGGAAAATGCAGAGGTCAAA |
| | | | GAGATGATGACTACATGGACTCTCCAGAAAGGAATCCCCCTGCTGGT |
| | | | GGTTAAACAAGACGGGTGTTCACTC |
| LNPEP | rs193993 | 73 | CAACACGGAAGAATCTATCATTTGGTGTGCATACTGCCAGTAGAGGG |
| Region | | | TGGGAGTTAAAAAGAAAATTTGGCCAGCAATTACCAGATCATTTTAG |
| | | | GCCAGCAGTGTAAATTCCTGTGTTATTTTTTGTCACATCATGCTTAT |
| | | | AATCATCTCAAAAGATAAAGTAATCATCATTACTCTGTGTTTATAAG |
| | | | TGAGAAAACTGAYACTAAGGGACAGATTTGCCCAAAGTCACCAAGTC |
| | | | AGTGAGAAAATCAGTACTTAAAATTTGTCTTCTAAGTCCAATAGTTA |
| | | | TTCAATTATATCACAGCTAGTTCCTAGTTTTAAGAAAAGTCCCCCAT |
| | | | CAATCTTCCCCTAAAGGTCCTAGATTTTGACCAACTCTCTTCTGACA |
| | | | CCAAAGGGCCCTGTAGTATTAAAAT |
| LNPEP | rs1974871 | 74 | TTTTTCGCCTCTTGCCCTCAACTCCAATGCATTTTCCTTACATAAAT |
| Region | | | TAAAAGGGACCATCAATTGGCATACAGTTCCAGGCTTAAAAAAATTA |
| | | | AAAGCTATATCCAGGGACTTATTTCGATAGTTCCTGAGTGTTACTCT |
| | | | GCTATTATTGTGCAGGTTCTATATACTCTTTAGTACAGATATACTAA |
| | | | ATCCCATTTATAYGTAATTCACTGCTGTACTTTAGATCAAAAGTCAA |
| | | | GGAAAGATTAATAACAGCCATCAACAATATTAATGTTGTTCTTGAAA |
| | | | AATGCAGTCTTAAAGAGCATATGAAATATCTTTAAGACTAACGGAAA |
| | | | AGAAGCATGCAGCTTAGGAAAAAATAGAGCACATATAAGTCCCACTA |
| | | | CTATAAAATCATCAATGCGATTTAG |
| LNPEP | rs1981846 | 75 | CCGGAGGGTGAGGCATGAGAAGCTGAGGCATGAGAATCACTTGAACC |
| Region | | | CGGGAGGCGGAGGTTGTGATGAACCAAGATCACACCACTGCACTCCA |
| | | | GCCTGGGCGACAGAGCAAGACTCCATCTCAAAAAAAAAAAAAAAAAA |
| | | | AAAAAAGACGTGGTTTTGTATAAGAAGTAAAAATAGTAAACAAACGA |
| | | | AATGTTTCAGAAKCCTACCTAGGAGCAAAAGAATAATAATGAAGTTT |
| | | | GTTTTGTTTCAACGGATACTGTTTTACATTTGACTTCATAGAGCCTT |
| | | | TTTGAGGGAATATGATATCACAATTTCACAACCAAAACCCATTATGT |
| | | | TTTTATCTTTAACACCCGCCTATCCTCCCACACAGAACTTCCTCTTT |
| | | | AGTTTAAGAATATGACAGTTTAAGT |
| LNPEP | rs2042383 | 76 | GACAGAGTGAGACTCTGTCTTAAAACAAAACAAAACAAACAAACAAA |
| Region | | | CAAAAAACATATAAAGATGCTCTTTACTATCCATTTCCATCACCCAC |
| | | | CGTCAGTGGTCCAGACACACTTTCTCCATGCTTCCGCTTAAGCTTCT |
| | | | CAGCACCAAGTATTGTGTTGCTTCTGTCTCTCATCCCTCTCCATTTC |
| | | | CCTCTCCCTTGCYATGTGTGTGTGCATGTATGTATATTTGTAGACAT |
| | | | CAGTTTAGCTCCCCTCCAACACGGAAGAATCTATCATTTGGTGTGCA |
| | | | TACTGGCAGTAGAGGGTGGGAGTTAAAAAGAAAATTTGGCCAGCAAT |
| | | | TACCAGATCATTTTAGGCCAGCAGTGTAAATTCCTGTGTTATTTTTT |
| | | | GTCACATCATGCTTATAATCATCTC |
| LNPEP | rs2042385 | 77 | CTGAGGCAGGAGAATGGTGTGAACCCCGGGGGGCGGAGCCTGCAGTG |
| Region | | | AGCCCAGATCGCGCCACTGCACTCCAGCTTGGGCGACAGTGAGACTC |
| | | | CACCTCAAAAAAAAAAAAAAAAGAAAAGAAAAGAAAAAAATGCCTTC |
| | | | AACTTGGTGATAAAAAAGCATCAAGTAATCATCTTTAAAAAAAAAAT |
| | | | CTTATAGCACCARTAGTGGCCACTAAATGATAGAATTTATATAAACC |
| | | | AGTAGTTTTGTATTTCAGAAAGCTTTTATATTTGTTGATTACATCAA |
| | | | CTTTCTATTTTCACCTCAGAGTAGGTTAAAATTTTATGCTTATTTTC |
| | | | TTGCTAACATTTTATCATCAGTAGGAGATAAAACACAAATAATTTGC |
| | | | AGAGTAAAAGCACATAAAATATTTT |
| LNPEP | rs210687 | 78 | CCACCACCCCTGGCTAATTTTTTTTTTTTTTTGTATTTTTATTAGAG |
| Region | | | ATGGGGTTTCACTGTCTTAGCCAGGATGGTCTTGATTTCCTCACTTC |
| | | | GTGATCTGCCTGCCTCAGCCTCCCAAAGTGCTGGGATTACAGGCATG |
| | | | AGCCACCGCGCCCGGCCTCTTTTTTGACTTTTTAACAATAATCATTC |
| | | | CGGCTGGTATGARATGGTATTTCATTGTGGCTTTAATTTGCATTTTT |
| | | | CTGATGATTAGTGATGCTGAACATTTATTCATGTTCGTTGGCCACTT |
| | | | ACATGTCTTCTTTTGAGAGTGTCTGTGAGACGGCATATTCTATAAG |
| | | | CAGTTGAGAATATCAGAGTACTAAGAAAATAATTCTGGAATAAGAAT |
| | | | TATAAGGCCTCTCACAGCTGTAATC |
| LNPEP | rs2113050 | 79 | ACTGAATTAAATAAGATGTTTATACCATTGAGTCATCCATTAAAAAC |
| Region | | | TAAAACATAAATAAAAGTATACCACAGTTATGAACAAGAAAGCTAA |
| | | | ATAAACAGGCTATTATATTTTTAAAAAGTTAGCTGAGATAATATACT |
| | | | AATTTCCTTAATATACTCCTGCCCCACAACCTGGGACCCTGCCCTGG |
| | | | GCTTGAGAGGGCMCTGTTTTGGCATTCCTCTGACTATGTCTGTCCCC |
| | | | ACCAGGCGAGGAGTCAGTAGGATCAAAGGGATGTGCCCACCTACAGT |
| | | | CCACGGTTCCCCTTCTAGGGTTTGGGCACTGCAATCCCAGAATTTCT |
| | | | GTCCAACAGGCCTCAAATCTGCTTCCAAGGTATTTGTGGGTCTCATC |
| | | | CCTGAGTCTCATTCTCTCTAGGGAG |
| LNPEP | rs2113189 | 80 | TTTTTTAATGTGATAGCACCTAGCATATGTTGATCTTATAATAGTGA |
| Region | | | TTAATAAGCAGTTAATGATTGATTAAAGAACTTATGGTCTGTCTTTG |
| | | | GGATTCATGTAGATAATAGGAAAGGCAAAGCAGAAAAATTCAGTTAA |
| | | | TTCAGATGATTCTAATAATTATTAAAATATTTTAAAATTTCCAACTG |
| | | | CAAAGAAAATAAWTTTTTATAGAACCATTAGACCCAGAGAACTCATA |
| | | | CCTCTAATTAGAAGAACCCTAAGTCATTGTAGACAGAAGAGATCCTT |
| | | | TCTTTTTTACAAGCACTTGTGTCCCAGGGACAGTAATAATATTGTTT |
| | | | AATATTTCTGCAGCAGTTTACAGTTTAAAGACACTTTCATGGCCGGG |
| | | | TACAATGGCTCACGCCTGTAATCCC |
| LNPEP | rs2113190 | 81 | CCCCACTGCTAGCTAAAAATATCTCAGCGCAAAATGTTTTTGAGTGG |
| Region | | | TTACTACTGCATTGGCATCCCTTAAGCTCTGAAAAATGCCAAAATAA |
| | | | GTATCCTGTTAGGTTTGGAAATACAGTATATCTTTTTCTTTTTCCTT |
| | | | ACCTCTGGGAAGTTATAGAATCACTACAGGAAAGAGAAAAGAAAGTC |
| | | | ATCACAGGGGAARAAAGGAAAACTTTTTATTTAAACAAAGAGTCATG |
| | | | CTAATCCCCTGAATATATATATACATAAATTTATATTTATTTATTTT |
| | | | AGACAAAGTCTCACTCTGTTGCCCAGGCTGGAGTATAGTGGCACAAT |
| | | | CTCAGCTCACTGCAACCTCCACCTCTCTGGTTCAACCAATTCCTCTG |
| | | | CCTCAGCCTCCCAAGTAGCTGGGAT |
| LNPEP | rs2113191 | 82 | AAGCCTGAAATCAGTTTTAGAAAAAAAAAAACTTAAAAAAAAACCTT |
| Region | | | TTAAATCTATTATTCTCTTCTTTTTGTTTCTGTTTCAATGGGTTGTA |
| | | | TGAGTGAAGCTAAAATGTAAACATCCTACTGCCCTATACAAAATAGA |
| | | | ATACTATTATTTCATCTTTATGCTAGTTACAAGAAAGATAATCTTAA |
| | | | CCTGCAGTAACCYACCTACAGTAGATATAAGTGTTCAACATGTTGAA |
| | | | TATACCTATGAAAATATTCTAGGTAAACTTATTTATGCTCACAATCA |
| | | | AAAATATGTGATTAAATATTGTTGGTTTTTTCTAAACTCCAAGATTG |
| | | | CTAGTATGAATTTTAATGAAGAATTTCTTTACATAGATTAATTGATT |
| | | | ACTTCATTCATTTGTGCATTTGAAA |
| LNPEP | rs2161548 | 83 | GTTCATACTTGCTTTCCTTTGTACATTGTTATCTCCAGTGTTTGAAA |
| Region | | | TTCTCTTATCCCCAGTTAATTTGTAATTGTCTTGAACACTACTTGAT |
| | | | AAAGTGTTCAACTTTAGTATTTCAGAAAGGAAATATACTCTCCAGTG |
| | | | AAAGAATAAGCTTCAAGTTATTCAGCATAGATAGAACAGGTTATAAG |
| | | | TATTATCAAGCARCAGCCTTCTCAAAGGGATTTTTATGTGAGATAGT |
| | | | TATGATTGGATCTCTTAACAACAGTTTAAGGCTTTTTCGCCTTAGTG |
| | | | TGTTATGATCTAATTTTTATCCAAAAGTGCTGGGTCTCTCTTGTATG |
| | | | AATAGTAGGGATAACATCAAACTTTATTCCCTGGTCCCTTATGCTTC |
| | | | TCTTCCAAAATCTTCTGTTAACTAC |
| LNPEP | rs2161657 | 84 | TCTAGAAAGTAACAGAATAATTGTGAATGTTTATAAATAGCTATATA |
| Region | | | TTGTCAGTCAACCATATTTATTCTGCTTGCTATGTTGTCATGGTCTA |
| | | | TAGAAGGCAGCACTTCCCTTTTTCCTCTAACACCACACTAGGATGTC |
| | | | ACGCTGGGGTGGCCCCAGGGGCTCACTATTTGACCTGACTTTCTTTG |
| | | | GTTTCTCTTCTAYGACTAATCCTACACTTTTATGTTCCCTTGATCTA |
| | | | AAATCTTTAAAATGTTGTAATTTCTACCATATAACACCTAATCTCTG |
| | | | GCAAATTTTAAGGTTGTCAACTGTATTCCCCAATGTGTATATATTTG |
| | | | TTGAGCAAACTAATCTTCTCTCTTATAAGAAGAAACTTGCTAAT |
| | | | CTCTAGATCATTGTGCCTATATTTC |
| LNPEP | rs2161658 | 85 | TATAATTGTAATCCAATATTTTAAAATGTGGAGAACTTTACCTAAAA |
| Region | | | ATCCTGACCTCTCGAATCTCTTAAAATGATATTTGGCAACCTGTGGG |
| | | | TCTTTATCCTGTGGGCCACAGGATAGCTATAAAGGAGAGTGCAGTGG |
| | | | GCCAAACTCTCTTTAGGCCAGGCCTACTTTCTCCCAAGCACCAAAGT |
| | | | CCCAAATGGCCTSTTTCATTCACTTAGTGTGGACTCTCTAAGTATTT |
| | | | GAGCTTTCGACCCCACATTTAAAATGTATTTATGTTATTAGCTGCTA |
| | | | CACCAAATACCGGTGTAATCTCTTAAGGAAAAGTACAAATATAGTCT |
| | | | TAACTTTTATAATTTTAAATGGGTTGTTATGAAATCTATTCATTACT |
| | | | TACACTGGAGAAATTAGTATGACGT |
| LNPEP | rs2247650 | 86 | AACTGATTCTAGCCACTTTACCAGTTAGCAAGACTAGTTTATTCATT |
| Region | | | CATTCAGTAAATACCTACGGAGAACCTACTGCGTTCCTGGCACCATG |
| | | | CCATATGTTAGAAATACAAAGATGTATATAATATAGCCACTGCCTCA |
| | | | ATACAAATGATGGAGGTCAACCAGTAGACAAATAAATACAGTAAAGC |
| | | | AGGTGCTAAGATMAATGTCTTTGCCAGGGACAGAGGGGATTCCAGGA |
| | | | GGAAATAATCAGTTTTGCCAGGACAGTGTTCATTTCCTGAGCCTTGT |
| | | | AAATAGTACTCAGGTATGCTGAATATCAGGTAGTAGGAGCAAAGGCA |
| | | | GAGTGATACAACCTGGCATGTTCAGAAAATGGCAGGTAATCTTGGAT |
| | | | GGCTAAAGCTTAGGTGTAGGCAAGA |
| LNPEP | rs2248374 | 87 | TTCATTATCATGTCTGGATGAATTATTTCATATAGCTCTTTTAATAA |
| Region | | | ATGCCTGCATCCATGGCTAATGTGCACGTTCAGCCAACTAATGATGC |
| | | | CTACATTGCAGTGCTCTTTTGTTCTTGTTTTGTAGAGTTGTTTAGAA |
| | | | AGTGATTTTACATCTGGTGGAGTTTGTCATTCGGATCCCAAGATGAC |
| | | | AAGTAACATGGTRAGGATAAAGAGAGTCACAGAGTAGAAGAGATCTG |
| | | | TGGAATAGCCTGACCTAGAGTGAGTATGACATACAGAGTAGCCCACC |
| | | | TGTCCCTTTTAAAAGCTGGAGAGAAAGAGAGCCCCCACGATTTTCTC |
| | | | TAAAACAAAACTGAAGGGGAAATGCTTGGGGTATTTAGGGGGACAAT |
| | | | GCTGTTGCTACTATATTTTTGTTGT |
| LNPEP | rs2255546 | 88 | TTTGGGCTAGGGAGTTTCAAAGTTGACTCCACTGAACTATTTGGATA |
| Region | | | CAAATGGTATTATTTATATGCTTTGAGAAACATTTGATTACACTTGG |
| | | | TTTGAGGCAACTGAGACATCTGCATGGAAGAACAAACATTGGATGAA |
| | | | AATGAATACCAACTTTTTCAGAAGATGGGTCCAATTTTCTCTTACAA |
| | | | AATCCCATGCTARTTGCTGCCCCTTTGGACGTCTGGCAATCGCATGA |
| | | | AGGAGAGCTGCCAAGTTCTGTGTCTTGATAACCTTTCCTTCCATTCC |
| | | | TAGTTCAATTAACCTGAAGAAAGAAAAATAATTTGTTTCTAAATAGT |
| | | | AGGTATTATGTACATGGACATTAACTCAAGCCACCAATATATTAAAA |
| | | | GAATAGAACAGAAAGAGGCATGATA |
| LNPEP | rs2255633 | 89 | GCCACATATGTGTGATGCACTATACAAGGCATCTTGGGATTCTTCTG |
| Region | | | GGCTGAGGAGAGAGCCAAGAAGGGATGGTGGGAGGGGGCTGATGACT |
| | | | GCATTTAATTCAACTCGGACTTGATGCCAGTGGTTTCTTGCCTGGAC |
| | | | TGAATGAGAGAAGGCTGTTTCCCATTCCCTTATTCTCATGTCTCTCC |
| | | | CTTACTCCTGGAYAGGATAATTTGGGCTAGGGAGTTTCAAAGTTGAC |
| | | | TCCACTGAACTATTTGGATACAAATGGTATTATTTATATGCTTTGAG |
| | | | AAACATTTGATTACACTTGGTTTGAGGCAACTGAGACATCTGCATGG |
| | | | AAGAACAAACATTGGATGAAAATGAATACCAACTTTTTCAGAAGATG |
| | | | GGTCCAATTTTCTCTTACAAAATCC |
| LNPEP | rs2255634 | 90 | CCAGTACAAAACTGCATTAACAAATGAGGCCACATATGTGTGATGCA |
| Region | | | CTATACAAGGCATCTTGGGATTCTTCTGGGCTGAGGAGAGAGCCAAG |
| | | | AAGGGATGGTGGGAGGGGGCTGATGACTGCATTTAATTCAACTCGGA |
| | | | CTTGATGCCAGTGGTTTCTTGCCTGGACTGAATGAGAGAAGGCTGTT |
| | | | TCCCATTCCCTTWTTCTCATGTCTCTCCCTTACTCCTGGACAGGATA |
| | | | ATTTGGGCTAGGGAGTTTCAAAGTTGACTCCACTGAACTATTTGGAT |
| | | | ACAAATGGTATTATTTATATGCTTTGAGAAACATTTGATTACACTTG |
| | | | GTTTGAGGCAACTGAGACATCTGCATGGAAGAACAAACATTGCATGA |
| | | | AAATGAATACCAACTTTTTCAGAAG |
| LNPEP | rs2255637 | 91 | TCTTACCAAAATTCCTGAGATTTTCCCCCAGTACAAAACTGCATTAA |
| Region | | | CAAATGAGGCCACATATGTGTGATGCACTATACAAGGCATCTTGGGA |
| | | | TTCTTCTGGGCTGAGGAGAGAGCCAAGAAGGGATGGTGGGAGGGGGC |
| | | | TGATGACTGCATTTAATTCAACTCGGACTTGATGCCAGTGGTTTCTT |
| | | | GCCTGGACTGAAKGAGAGAAGGCTGTTTCCCATTCCCTTATTCTCAT |
| | | | GTCTCTCCCTTACTCCTGGACAGGATAATTTGGGCTAGGGAGTTTCA |
| | | | AAGTTGACTCCACTGAACTATTTGGATACAAATGGTATTATTTATAT |
| | | | GCTTTGAGAAACATTTGATTACACTTGGTTTGAGGCAACTGAGACAT |
| | | | CTGCATGGAAGAACAAACATTGGAT |
| LNPEP | rs2278018 | 92 | TGATAGCTAATTTCTTTGGGGGAGCCATTGAACATATTTGAGCATTT |
| Region | | | CTTAGTTTAAAGCAAGCTTGACAGAGGGCGGATCCAATAAGTTCTTC |
| | | | ATGGCTTCCCACATAGGTCTAGAAGAAACCTATGTTTTATTTGAATT |
| | | | GTGTTTGTGGTCATCATTTTGGAAAGCTAGTTGACAAGTGTTAAAGT |
| | | | ATATCATAGAGAYGAACTCAAGTGGGTTCCTCTCTATGATATACTTG |
| | | | GATTATGATACAATGGGTTAACATGATTTGAAAGTTACAGATGAGCA |
| | | | CTGAGGAAATGGATTGTAACAGAAGATTCAGGGTGTTTGTAATTTTC |
| | | | AAGACTGACTTTTGCTTTAATACTTCACAGAACCTCTTATCCTTATT |
| | | | GACAACATGCTTAATGGTATCTTAA |
| LNPEP | rs2278019 | 93 | AAACTTGCTTTGATCTCTTCCCTCTTTCTCTTTCTATGTGATTTAAA |
| Region | | | TGAGCACTGAGGAATTCAGTTAGCTCAGGAAAAAATAATTTGTTCCT |
| | | | CAGAGATGATTCTTGAGTGTAGAAAATAAAATATTTATGACATGCCC |
| | | | CAACAGTGTGGATCATTTCTCTATTCTTTTATCAGGTTTAACGTTAA |
| | | | CATATCTGCATCRAACTCTTTCCCAGGCTGATCAGAAAGGGCACACA |
| | | | CTGGACGCTGGGACGGAAGCCAGGTAGAGGGTCCAGGAAAGAGATGG |
| | | | GGAGAAAAAGAAGGAACACAGTGACTGCTCTGTTCAAAATAGGGGTC |
| | | | CACATGTCCAAGATGCTGTGGCTCCCTGTGGCGGACATCAACGCTCT |
| | | | CATCCATTATGCTCCTCTTCTGTGG |
| LNPEP | rs2287988 | 94 | CATCCAAGGAGATGGAAGTTTCTGTCCCTCTGTCTTTGGATTGTCTC |
| Region | | | CTCTCTCTTGAGTTATCATAGTCACCTGTCATTTCAGCTCGCCTTTC |
| | | | TGGGGGAAAATGCAGAGGTCAAAGAGATGATGACTACATGGACTCTC |
| | | | CAGAAAGGAATCCCCCTGCTGGTGGTTAAACAAGACGGGTGTTCACT |
| | | | CCGACTGCAACARCAGCGCTTCCTCCAGGGGGTTTTCCAGGAAGACC |
| | | | CTGAATGGAGGGCCCTGCAGGAGAGGTGGCTGCTTTTCTTCTTTAGG |
| | | | TCTAGCTTACCTCATCTCAGTTTCCTCGTTATTTCCTTAGCTTTCTC |
| | | | TCAGCTCATCTGGCAACTTTGTAGGATGCTAGTTCCATATAAGAATC |
| | | | AAAGGCCTAAAGTAGACTTGATAAG |
| LNPEP | rs2303208 | 95 | GGGAAAGGGGCCATTATCTGGTATTTTACTTAAAAGCACAGAAGTTG |
| Region | | | AATTGATGCCAGTGTTGGAAATTATTGCATTTTAAGAAAATAGAAAT |
| | | | ATGTAATATTTTTATGCTTTCAATCAACAAAATGAGATTTGGCATTT |
| | | | TTGTGCTTTGGGGATCTCAAAAGCAGGGCTTTTTGTTTTCAACAGAG |
| | | | TGTTGGGGTAAARGCAATGGAGGTAAGAGAGGCTACAGAATACTAGG |
| | | | AGAGGCCATTGCCCCCCTAGGAGGTCATCGATTGTCCTTCAGAGTAT |
| | | | GAGGCTTGCCTCTAACTCACCTGCCATAAGTCATAGGCATGGTTATG |
| | | | AAATACTCCAGTTTTCAAGTACTGATTATTCCTTTTCCTTTCTGTAG |
| | | | GTTAAGACAATTGAACTTGAAGGAG |
| LNPEP | rs2303209 | 96 | GAAAGGGGCCATTATCTGGTATTTTACTTAAAAGCACAGAAGTTGAA |
| Region | | | TTGATGCCAGTGTTGGAAATTATTGCATTTTAAGAAAATAGAAATAT |
| | | | GTAATATTTTTATGCTTTCAATCAACAAAATGAGATTTGGCATTTTT |
| | | | GTGCTTTGGGGATCTCAAAAGCAGGGCTTTTTGTTTTCAACAGAGTG |
| | | | TTGGGGTAAAAGYAATGGAGGTAAGAGAGGCTACAGAATACTAGGAG |
| | | | AGGCCATTGCCCCCCTAGGAGGTCATCGATTGTCCTTCAGAGTATGA |
| | | | GGCTTGCCTCTAACTCACCTGCCATAAGTCATAGGCATGGTTATGAA |
| | | | ATACTCCAGTTTTCAAGTACTGATTATTCCTTTTCCTTTCTGTAGGT |
| | | | TAAGACAATTGAACTTGAAGGAGGT |
| LNPEP | rs2351010 | 97 | CGAGTTATGCTTGGTATAGCTATTAGGTAATTCAATTAACTTGCAAA |
| Region | | | ATAGATGAAGAAAGCAATTCTGAGAAGATCAGCTGAAATCACTGGAA |
| | | | AAACTCAAAAAGGCAAGCCACTAAAATTGTTGTTGAATTAGGTAGGA |
| | | | GACAACTCTAAAAGACTGGGGAAAAAAATTGAAAGTCTAGACAAATT |
| | | | TTGCACTCGGACYGCTTCACAGGTGTCCACATTTTCATTTCACTTTA |
| | | | TAATAGGTTATAGGGTCAAAAACTTGCTGAAGCAAAACACACAGGGG |
| | | | TGAGTTTCTGTTTTAATATTGTTCCATTTCCTTTCTCTACTTTGCCC |
| | | | TGAAGGCAGGCTTTGGTCACAGAGTGGTGTGGAAATGCCAACAGGTA |
| | | | AAATTCCAATGAGTCCCATATTTCT |
| LNPEP | rs2351011 | 98 | TCAGCTCACTGCAACCTCCGCCTCCTGGGTCCAAGTGATTCTTCTGC |
| Region | | | CTCAGCCTCCCAAGTAGCTGGGACTACAGGTGCGTGCCACCACATTC |
| | | | TGCTAATTTTTGTATTTTTATTAGAGACAGGGTTTCACCATATTGGC |
| | | | CAGGCTGGTCTCGAACTCCTGACCCCATGATCTACCTGCCTTCGCCT |
| | | | CCCAAAGTGGTGKGATTACAGGTGTGAGCCACTGTGCCTGGCCAAGT |
| | | | GTCAGGTTTTAATCCTGTCCCTTCCATTTACTTGCTATATGGCATTG |
| | | | GACAAACAACTTTTTTAAAAACTAAAATGAGAACTTCAAATCAGATT |
| | | | ATATCTAAGTTTACTTTCAATTCCACAATTTGAACATTTATTTTGAA |
| | | | ATTGTTAAAAACAGAAAGTCACAAA |
| LNPEP | rs248215 | 99 | ACATTTTAATGTATATAAATATTTGCTACATTCTGTGTGTTATATAA |
| Region | | | TGTGGTACCCAGTCCTCTGCTGGGACATGGATGTACATAATGAAACA |
| | | | TGGAGGTCCAGACGTATGATAACTCTCCTGTTTCCCTTCCCTCATTG |
| | | | CCTACAGGGGCAATAGTTTCATATCTTGGGTTTTTTATTGTTTAATT |
| | | | TTTTTTTATGGGRAGGGGTTCTTTGGGTGGGTAATAGTCAGGGGGAA |
| | | | AGGACAGTGTCTATACTTTTTAAAGATGTATATAAATGTTTCATGTT |
| | | | ATTGGTTTTGTACCTAGTCCTTTGCATGGATATATAGGTACCTAATG |
| | | | AAAATCGAGGATCAGTGTATGACAAATCTCCCATCCTCCCCTTTCCT |
| | | | TATTGCCTGTGTCGGCAATAGGAAG |
| LNPEP | rs251339 | 100 | ATCATTACTCTGTGTTTATAAGTGAGAAAACTGATACTAAGGGACAG |
| Region | | | ATTTGCCCAAAGTCACCAAGTCAGTGAGAAAATCAGTACTTAAAATT |
| | | | TGTCTTCTAAGTCCAATAGTTATTCAATTATATCACAGCTAGTTCCT |
| | | | AGTTTTAAGAAAAGTCCCCCATCAATCTTCCCCTAAAGGTCCTAGAT |
| | | | TTTGACCAACTCYCTTCTGACACCAAAGGGCCCTGTAGTATTAAAAT |
| | | | AATAAATTACTGAAAATATCTTGCCCACCATTGTGTCACATAAAGTC |
| | | | AATTCTAATACATGTCAATAGCAACTTGAGAATGAGAAGAATTAGTT |
| | | | GCTGTTATTTTTCATAAGATCATTTAAAGGCATTTGAGAGCCTTAGC |
| | | | ACATTCTTCATTTTTTCTCATTTGC |
| LNPEP | rs251340 | 101 | GTATGCTGTTAGGTTTGGAAATACAGTATATGTTTTTCTTTTTCCTT |
| Region | | | ACCTCTGGGAAGTTATAGAATCACTACAGGAAAGAGAAAAGAAAGTC |
| | | | ATCACAGGGGAAGAAAGGAAAACTTTTTATTTAAACAAAGAGTCATG |
| | | | CTAATCCCCTGAATATATATATACATAAATTTATATTTATTTATTTT |
| | | | AGACAAAGTCTCRCTCTGTTGCCCAGGCTGGAGTATAGTGGCACAAT |
| | | | CTCAGCTCACTGCAACCTCCACCTCTCTGGTTCAACCAATTCCTCTG |
| | | | CCTCAGCCTCCCAAGTAGCTGGGATTACAGGCACACACCACCATGCC |
| | | | CGGCTAATTTTTTTGTATTTTCAGTAGAGATGGGGTTTCACCATGTA |
| | | | GGCCAGACTGACCTCAGGCAATTCG |
| LNPEP | rs251342 | 102 | ACAGGCATGATCCACGGCGCCTGGCCCTAAATTGTGTTTTCTAAAGG |
| Region | | | AAGGTTCAGCATCATCCAGTGATCAGAAACCCATACTAGCAGTGCAG |
| | | | CAGCCAGAGGTTTTGTTCTCCATTCACTACACCTCTATTGATATTAA |
| | | | ATTGTTCTGTTGAAATATTTAAAGCTTCCCTAAAGACAGATATTTCC |
| | | | CTCGTAAACCACYCCTCCTGGATTCTACTGTTATTTGAGGGTTTTGT |
| | | | TTGTTTGTTTGTTTGATTTTGTTTGTTTGCTTGTTTTTGAAAGGGGT |
| | | | CAGGAAAGAGACTCCAGTCTCAACCTCCTTTTCACTGGCTTTTCCTG |
| | | | CCATGTATTCACCCTACTATATCCTGTATATATCCCTCAATTCAAGT |
| | | | AATTTGCAGAGAGCAGCCCTGGGAT |
| LNPEP | rs251343 | 103 | AGAATAAATTGTCTGTGAAAATACTGAAAACATACAAAGGACATTTT |
| Region | | | TTTCTCAGTTTTAAAACTGTATTCCGCTTTAAAAACTGTTTTCTAGG |
| | | | CCGGGCGCGGTGGCTCACGCCTGTAATCCCAGCACTTTGGGAGGCCG |
| | | | AGGCGGGCGGATCACAGGGTCAGGAGATCGAGACCATCCTGGCTAAC |
| | | | ACGGTGAAACCCYGTCGCTGCTAAAAATACAAAAAATTAGCCGGGCG |
| | | | CGGTGGCAGGCTCTTGTAGTCCCAGCTACTCGGGAGGCTGAGGCAGG |
| | | | AGAATGGCACGAACCCGGGAGGCGGAGCTTGCAGTGAGCCGAGATCA |
| | | | GGCCACTCCACTCCGGCCTGGGCGACAGAGAGAGACTCCGTCTCAAA |
| | | | AAAACAAAACAAAAACAAAAAAAAC |
| LNPEP | rs251344 | 104 | AGCTGGAGTGTCACGATCGTAGCTCACTGTAACTTTCAGTTTCTGGG |
| Region | | | TTCAGGTGATCCTCCTGCTTCAGCTTCCCACGTAGTTGTGACTGAAG |
| | | | ATGTGCACCACAATGGCTGTCTAATTTTTATTTTTTAATTTTTGTAG |
| | | | AGATGGGGGTCTCACTATGTTTTCCACACTGGTCTCAAGCTCCCGTC |
| | | | CTGCCGCCTTGGSCTCACAAAGGGCTAGAATTACAGGTGTGAGCCAC |
| | | | CACGCCAGGTCCTGGCTGTTTGGATTTTAAGACCAAGGGAAACATAC |
| | | | TATTTGTTGACCCCTCGGTCTCACTGAGGACCTGGAAAGAAATAACA |
| | | | GCAACAGTGCTGGCCTTGCAAATCCAAACCTAAAACGTGCTGTCTAA |
| | | | AAAAAGAAACTTCAGGCGGGGCGTA |
| LNPEP | rs2548225 | 105 | GACATGTTTTGCTGAGTGTGGTTGGTTACTTCAGGTACAGTATCACA |
| Region | | | TTAATAGGGGCCACAGTCTATATCCCTGTTTAGTTTGTTAGTTACCA |
| | | | CTCAAGAGCAGACAGATATTGTCCACTTTATCCCAAATCCCAGCCAG |
| | | | ACTTTGTTGTATGCTGTGCTTCATTCATGGGGCTGTGAACTACTGAT |
| | | | TATATTCTCCCTWTTCCTAATGTAGAATGCTTTATTCTACTGCCATC |
| | | | TTTCTGTCTGCACTGTTTAATTAGGCTTACTGATAACAACTTTAATT |
| | | | CTGAATTTTCTTTCTCATTCAGGTTCTATTTGTAATTACTAAGACTT |
| | | | AAAGAATAGTCTGGTGAAGTTACTCGAAGAATTAAGGAAGGTTTGAG |
| | | | CTAAAATGAACTAGAGACCATCTAG |
| LNPEP | rs2548516 | 106 | CACATTTTTGAGAAGTGATGCTAAAAATTTTTTTTTAAAAAGACTCA |
| Region | | | CATATCTATAGAACAATTGTTATTTGTAAGATTAAAAGATGGAATCA |
| | | | CAATTTTATACTGTATTACAACCCACAAATATCTCATTTGTTGCCCA |
| | | | GACTTCCCATTTTTGAAGTTGAAAAATACTTTCAACTGGATACCAAT |
| | | | CTGAACATGAAARCAAAAATAATTTTTTAAGACAACTAAGTCCTCCT |
| | | | TGTTTGATTATGCACCACACTGCGGTAATAAAAGTGATTCATAGGAC |
| | | | CTACATTCATATGAAAAAAAAAACTATTTATGTTTTCAGTTCTGGGA |
| | | | CCTCAAAATGCCAAAATACCAACCTTTAGATATACTTTAGAATATAT |
| | | | CACAAACTAGAAAGTATATATACTT |
| LNPEP | rs2548520 | 107 | ACTATAAAATGAAAATGTAGATACAGCTTCTTTGGGAAATATGGTTA |
| Region | | | GTTATTCAGCAATGTTTATGTTTAATTTTATGTTTCTGTTTAAAGCA |
| | | | GTGTTCCCTACTTTTTTTGTTACTACATACTCCAGTCAGTAAAGATT |
| | | | TTTTGAGCAAGAACTTCCCAATATACATATTTTTATTTATAAATTAT |
| | | | ATGGGCTGGGCGYGGTGGCTCACACCTGTAATCCCAGAACTTTGGGA |
| | | | GGCCGAGGTGGGTGGATCACCTGAGGTCAGGAGTTTGAGACCTGCCT |
| | | | GACCAGCATGGAGAAACCCTATCTGTACTAAAATACAAAAAAATTAG |
| | | | CTGGGCATGGTGGTGCATGCCTGTAATCCCAGCTACTCAGGAGGCTG |
| | | | AGGCAGGAGAGTCACTTGAACCCGG |
| LNPEP | rs2548521 | 108 | TATGTTTCTGTTTAAAGCAGTGTTCCCTACTTTTTTTGTTACTACAT |
| Region | | | ACTCCAGTCAGTAAAGATTTTTTGAGCAAGAACTTCCCAATATACAT |
| | | | ATTTTTATTTATAAATTATATGGGCTGGGCGCGGTGGCTCACACCTG |
| | | | TAATCCCAGAACTTTGGGAGGCCGAGGTGGGTGGATCACCTGAGGTC |
| | | | AGGAGTTTGAGAYCTGCCTGACCAGCATGGAGAAACCCTATCTGTAC |
| | | | TAAAATACAAAAAAATTAGCTGGGCATGGTGGTGCATGCCTGTAATC |
| | | | CCAGCTACTCAGGAGGCTGAGGCAGGAGAGTCACTTGAACCCGGGAG |
| | | | GTGGAGGTTGCAGTGAGCCAACATGGCGCCATTGCACTCCAGCCTGG |
| | | | GCAACGAGAATGAAACTCCGTCTCA |
| LNPEP | rs2548522 | 109 | ATAAATAATGTATTTATTTAACTTTTTATACTATATATCATTTATGT |
| Region | | | TTATAAATTTATAACAATATAAAATTTAAAATTAGATAAGAAATAAT |
| | | | AGACACTCTAATGTATTGCACTTCTTGCACACTTCCTCATCCCATTT |
| | | | TGGATACCACTGTGTTTAAACATTGTGTTTAGTGGGGCAATGTTACT |
| | | | TGGTTGAACTCTYATTCACGGCCACAGAATGCATCCTTCAACCCAAT |
| | | | GTTTTATATATGGAAAACTTGTACTACAGGTCAAAGTGATTCATGTT |
| | | | GATAAAGCAGAGCACAGTTACAGCTCAGAAAAAAATATGGTTCCAAG |
| | | | TCTAGGCTCTGCACATGGTTGGGCAGGGGCATCATTTCCTGTTTAAA |
| | | | ATGAAATCCACAGCATTGTAGATTA |
| LNPEP | rs2548523 | 110 | TGATTTTTATTATCTTGAGGACATTAACCAGTTTTTATTCTAAGTGG |
| Region | | | GCATATGACTTTTTAATCCCAAATGTCAACGGATCAATAAGAACTGT |
| | | | TATTGATGTCTCAGAAAAAACACAACACAGAGCATTGAGGAGGGAGC |
| | | | CAGAAATTTGCATTTGTCACAAAGTGACCATATGGTAGAGATTGTAC |
| | | | TAGTCTCCATACMTCTTATTTAACTGTCAAAAGCTACCCTCTGAGAT |
| | | | AGTTATTATCCTGATTTTTTTAAGCGGAAATAAATCTCAGAAAAGTT |
| | | | AAGTAACTTGCACAAGATGACATAATTTGCCATTTGCAGATGGGATT |
| | | | TAACCCTATGATTCTAATGCTTTGGCTACTTCCTCTATACTATATGT |
| | | | ACTTAAATACCCCAAGTGACATTTG |
| LNPEP | rs2548524 | 111 | ATTCTAATGCTTTGGCTACTTCCTCTATACTATATGTACTTAAATAC |
| Region | | | CCCAAGTGACATTTGAATAATATAATAAAGATCAAATAATTATAATA |
| | | | CATATTGTTTTCATTTTAGTGTATTTTGCTGAACAACTTTATAACAA |
| | | | TTGGTAACAAACACATTGTAAGCTTCTTGAAGGTAGGCCACATGGTT |
| | | | GTTTTGTTCACCWCTTTATCCTTAGCTCCTACAGCAATACCTGGCTG |
| | | | GCATAAAGGAAATGTGCAACTAGTTACATTTCAAATCCACAAATGAA |
| | | | TGAAGTAATCAATTAATCTATGTAAAGAAATTCTTCATTAAAATTCA |
| | | | TACTAGCAATCTTGGAGTTTAGAAAAAACCAACAATATTTAATCACA |
| | | | TATTTTTGATTGTGAGCATAAATAA |
| LNPEP | rs2548526 | 112 | TTTGCATTTTTAGTGCAGACAGGGCTTCACTATGTTGGCCAGGCTGG |
| Region | | | TCTTGAACTCCTGACCTCAGTGATTCACCCTCCTTGGCCTCCCAAAG |
| | | | TGCTGGGATTATAGGCGTGGGCCACTGCATCCAGCCAGCACCGGAAT |
| | | | AATGGACACCATTACATTTCATGGTAGAGATTGTACTAGTCTCCATA |
| | | | CATAGCACTTACRATGTGAGAGCATGAAACAGGGTTTGTAATGCCCA |
| | | | GCATGTTTTTTTTTCTATCCTCACTAAAAGGTTTTAGACCTAATGTC |
| | | | TTGCTTGATCAAAGACTTTTACATCAAAGAGAAAAGAACAAAGGGTA |
| | | | GGACAGAAGTCTACATCTACTTTAATTTTCCACTGGATTCCATCCAC |
| | | | TGGGAGAAGAGTTCAGCAGCTTTCT |
| LNPEP | rs2548527 | 113 | GTCCATCATGTGGTAAAACGATTCCAAGTAACTCAGACCTTCGAGAA |
| Region | | | GTGCGGGGCTGCTTGTTTCATGTTGGAGGTAGTAAGTCATGTCAAGA |
| | | | GCTTTGTCTAGGGTCAGTCTCCCTGCACTGAAGTATAAAACAAATGT |
| | | | CAGTGGTTTGTGCATATCTTATAGTTTTTAATATTTTTAACATTAAA |
| | | | ACAAATATGAAAKAGAGAACAATAACAGAAGTAGGTCATTATAGCTG |
| | | | GGCCATTCAGTTTAAATCTTAGCTCTGCCACTGACTAGCTGAGTAAT |
| | | | CTTGGACAAATTACCAAACCTCTCTGGACCTATTGCCTTCTTTATAC |
| | | | AATGGGGATAATAATACTATCTTCCTCATAGGCTTGTTGTGAGGATT |
| | | | AATGACCTAATATTTTAAAAAGCAT |
| LNPEP | rs2548529 | 114 | TTTTCCAAGTCTTGCATTGTTAATCTATCTTCTCTTCAATCTACTCA |
| Region | | | GAGCTTCTCCTTTGAGCAGGCAAACCTCCTTCAACTAGCAATAGCTT |
| | | | GCTTCTTAGCTCACCTTTCATGCATTCACTCATATGTAGGATGGTGT |
| | | | TTTTCCTAATTTTGGCCTCCAATGAGCTATGCCTCCAAGATGTTCCT |
| | | | GAGACCCACGGGRCTGAGGATTAAGCCTCACTTCCTCTTTTCTTTCT |
| | | | AACTAGAGGATTTTAGTGTACCGAGAAACTTTGTCTCAGAGATGTCC |
| | | | TGCTGGTCACTTTCACTCAATTTGACCTAAATAAGCTCCTGAAAGAA |
| | | | AATTATATGTCACATTGAGTAAAGTGAGTGCATCACAGTAATTCTCA |
| | | | GAATAGGGAAAGCTTGCAATGCACA |
| LNPEP | rs2548530 | 115 | TTCACTCAATTTGACCTAAATAAGCTCCTGAAAGAAAATTATATGTC |
| Region | | | ACATTGAGTAAAGTGAGTGCATCACAGTAATTCTCAGAATAGGGAAA |
| | | | GCTTGCAATGCACAATATACTTCCTGGTCCTCAATTCCCTCCAGCCA |
| | | | TTGGTGATCTTGTGACCTGATTCATTCCACACATTATTCTGTTCATG |
| | | | ATACATAGAAAARGAAAGAAAACACTTTGTCCTTCCAGGAAAGTCTA |
| | | | GAGAGGAATGAATGCAAACAGCCATTTATTACTTCATTTCCCTACAA |
| | | | ACGTCATACTAATTTCTCCAGTGTAAGTAATGAATAGATTTCATAAC |
| | | | AACCCATTTAAAATTATAAAAGTTAAGACTATATTTGTACTTTTCCT |
| | | | TAAGAGATTACACCGGTATTTGGTG |
| LNPEP | rs2548532 | 116 | GGAATTTGGCTTAATTTGATGATGTCCTTGTCTCAAGGTTTAGTCAC |
| Region | | | TAGTCATTGAAATATGTATGTGTAAATAGGTGATCCATTTGTTCATT |
| | | | TTAGTAAAGAAGGACTCCAGGTTAAGCATGACTTTGTGACGGAAAAC |
| | | | CTCTCAAATTTTATTAAAGTGTTTAGAAGAAAATTAAAATTATACTA |
| | | | TGTATTTTTAATRTGGCATATATTATACTAGAGGGTAAAATTACATT |
| | | | ATAATATTCTCTCAACAACTCTGTGAGGTTTAGTCTTTATTCAACAT |
| | | | AAGATGAAAAAATTGAAGCTCAGGATGAGTGTGTACATTTTCTTAAG |
| | | | GTCACACATCTAATAAGTGAGAGAGTGAGGACTTGAATCCAGAAGCA |
| | | | ATCAATTTTAAAGTATGTGCTTTTT |
| LNPEP | rs2548533 | 117 | AAATTATACTATGTATTTTTAATGTGGCATATATTATACTAGAGGGT |
| Region | | | AAAATTACATTATAATATTCTCTCAACAACTCTGTGAGGTTTAGTCT |
| | | | TTATTCAACATAAGATGAAAAAATTGAAGCTCAGGATGAGTGTGTAC |
| | | | ATTTTCTTAAGGTCACACATCTAATAAGTGAGAGAGTGAGGACTTGA |
| | | | ATCCAGAAGCAAYCAATTTTAAAGTATGTGCTTTTTTCCACTGAACA |
| | | | TTTTTTGCCTTATCCATAACCTGTAAAAATAGATTAGTGGGTATTAT |
| | | | AAGACATAAGATAGATTTCTGTTATTTCTTGATGTAAATAATCTGTC |
| | | | TCTAAATGATAAAAGCGCAAGAGAACTTCCCACTGAATGAAAAATCC |
| | | | AGATTTTCTTACTAAAAGAGTTATT |
| LNPEP | rs2548534 | 118 | CCTGCCTTAGCCTCACGAATAGCTGGGATTACAGGCAAGCACCACCA |
| Region | | | TGCCAAGCTAATGTTTGTATTTCTAGTACAGACGGGGTTCCACGAAT |
| | | | TGGCCAGGCTGGTCTCAAACTCCTGACCTGAAGTGATCTACCCACCT |
| | | | TGGTGTCCCAAAGTCTTGGGATTACAGGCGTGAGCCATTGTACCCGG |
| | | | CCATGAAAGTGTYTTTAAACTGTAAACTGCTGCAGAAATATTAAACA |
| | | | ATATTATTACTGTCCCTGGGACACAAGTGCTTGTAAAAAAGAAAGGA |
| | | | TCTCTTCTGTCTACAATGACTTAGGGTTCTTCTAATTACAGGTATGA |
| | | | GTTCTCTGGGTCTAATGGTTCTATAAAAAATTATTTTCTTTGCAGTT |
| | | | GGAAATTTTAAAATATTTTAATAAT |
| LNPEP | rs2548535 | 119 | CTTAAAATTTGCCAGAGATTAGGTGTTATATGGTAGAAATTACAACA |
| Region | | | TTTTAAAGATTTTAGATCAAGGGAACATAAAAGTGTAGGATTAGTCG |
| | | | TAGAAGAGAAACCAAAGAAAGTCAGGTCAAATAGTGAGCCCCTGGGG |
| | | | CCACCCCAGCGTGACATCCTAGTGTGGTGTTAGAGGAAAAAGGGAAG |
| | | | TGCTGCCTTCTAYAGACCATGACAACATAGCAAGCAGAATAAATATG |
| | | | GTTGACTGACAATATATAGCTATTTATAAACATTCACAATTATTCTG |
| | | | TTACTTTCTAGATTAAATAACAGTCTATCGTTACCCAACATATGACT |
| | | | TACATTTGACAGACTGCTCCACAAGTCATCATTCTTAGCATTTCTAT |
| | | | AGCTGAACTTCTTTAAGTACTGAAT |
| LNPEP | rs2548536 | 120 | AATAACAGTCTATCGTTACCCAACATATGACTTACATTTGACAGACT |
| Region | | | GCTCCACAAGTCATCATTCTTAGCATTTCTATAGCTGAACTTCTTTA |
| | | | AGTACTGAATTATTCCTTTCTGGAATTTCTCCTCACCCAGAAAATCC |
| | | | TTGAGCATATTCAAAATACAAGCTCCCTTTAAAAAAAAACAAAAGAG |
| | | | TTGAAAAAAGAGWTAAAGAAAATGGTAGTATGGTATGTTTTTAAAGG |
| | | | AAGCTTAAATTTTACGGAACATGTGTGATGTCTGAAAAGTGAACAAA |
| | | | TAAAAAGTGAAACAAGTAGCAGGAACTGGCACCAGTGACTTAAACTG |
| | | | CTGATTCTATAGTCATTATTACACTTCTGAAAGCAGAGCTTCCACCT |
| | | | GCACCTGATATTTACTACCTTGTTA |
| LNPEP | rs2548537 | 121 | CAAAAGAGTTGAAAAAAGAGATAAAGAAAATGGTAGTATGGTATGTT |
| Region | | | TTTAAAGGAAGCTTAAATTTTACGGAACATGTGTGATGTCTGAAAAG |
| | | | TGAACAAATAAAAAGTGAAACAAGTAGCAGGAACTGGCACCAGTGAC |
| | | | TTAAACTGCTGATTCTATAGTCATTATTACACTTCTGAAAGCAGAGC |
| | | | TTCCACCTGCACSTGATATTTACTACCTTGTTATAGGAAACTTCATC |
| | | | AAACATTTCCTGTATTTGAGTCGGGGTTTCCGCTGGTTTGGAGATAG |
| | | | GGCGGGATGAATTCAATGAATCTTTTGTAATTACTTCAAAACACACA |
| | | | TTCAAAAAATAGTCATCCTAAACAGGGAGAAAAATGTTTAGTTTTAG |
| | | | TTTCTATTTGACACTGTAAAAGCAA |
| LNPEP | rs2548538 | 122 | TGATGTCTGAAAAGTGAACAAATAAAAAGTGAAACAAGTAGCAGGAA |
| Region | | | CTGGCACCAGTGACTTAAACTGCTGATTCTATAGTCATTATTACACT |
| | | | TCTGAAAGCAGAGCTTCCACCTGCACCTGATATTTACTACCTTGTTA |
| | | | TAGGAAACTTCATCAAACATTTCCTGTATTTGAGTCGGGGTTTCCGC |
| | | | TGGTTTGGAGATWGGGCGGGATGAATTCAATGAATCTTTTGTAATTA |
| | | | CTTCAAAACACACATTCAAAAAATAGTCATCCTAAACAGGGAGAAAA |
| | | | ATGTTTAGTTTTAGTTTCTATTTGACACTGTAAAAGCAATAGAAAAC |
| | | | ATAGTAGGTTTAGTAAGATGTTCTTAGAGGTAAGATTTCAATCGATA |
| | | | TTTCTTGGGAGATGTTTCTTTTCTT |
| LNPEP | rs2548539 | 123 | TAAGAATATATAAAGCTTTGGCATTAAGCCACAAATTCAGTACATAC |
| Region | | | ACAGTAACAAGAAGAGCCTAACTTTGAATCCATGTCTGTCTATAGTG |
| | | | TACTGGACTAAATATATATCCCAAAGACCTAATTAACCATTACTAAC |
| | | | CACCTTGATATGCAAATTTGTGTAGTGTTCAGACCACTATATTCGTT |
| | | | TTTAAAAAAGACRTACCTGAAGAAATCCCTTCACACATTTTGGGGAA |
| | | | GCCAGCAGACCCTTTGGAAATTCTAGAAAAGTACACCCCCAATGATG |
| | | | TTGATTTCAGGTTACATGCCGAGAACTCTCTATAGTACCTAGTAGCC |
| | | | ATGAGCATTCCTGTGCAGATGTATACAAACAGTGATGTTCTTTCCTC |
| | | | TCAACCCACATACACAGTTCTACAT |
| LNPEP | rs2548540 | 124 | GTCTAAGATTTAAAAAATATATAAATCAAATAAAAAGGATGCATAAA |
| Region | | | TATAATTGACATATTTACCCTTTACCTATATTTGATCTGTATTATGC |
| | | | ATTTTAAATATTACTATTCTTTTATGTGCTTTTATGTTTTATTTATT |
| | | | TAGTCTATATGCCTAATACTGCACATCTAGTGTATGTCTCTAAGATT |
| | | | AAAATCTCTAGAYGGACCAAAGCTTCAACAATAAGATTCTAAGATTC |
| | | | AGAAGAGCCTGGTTATAGTTACAGAACAGAAAATTATAAGTCTGTAG |
| | | | CTTCTAGAAACAGTTTAAGCACTATTCCTTTTCTGACAGTCTTCAGA |
| | | | TTACTTTACAAGTGGGCAGCAATCTTCTGAAGGGCATTCATGGAAAG |
| | | | GGAGAGGTGTTTCCTCAATTTGAAA |
| LNPEP | rs2549781 | 125 | GCTAACTGGTAAAGTGGCTAGAATCAGTTTATCCTGTACTTCTTATA |
| Region | | | TTCACCGGTTTTCAAATTGAGGAAACACCTCTCCCTTTCCATGAATG |
| | | | CCCTTCAGAAGATTGCTGCCCACTTGTAAAGTAATCTGAAGACTGTC |
| | | | AGAAAAGGAATAGTGCTTAAACTGTTTCTAGAAGCTACAGACTTATA |
| | | | ATTTTCTGTTCTKTAACTATAACCAGGCTCTTCTGAATCTTAGAATC |
| | | | TTATTGTTGAAGCTTTGGTCCGTCTAGAGATTTTAATCTTAGAGACA |
| | | | TACACTAGATGTGCAGTATTAGGCATATAGACTAAATAAATAAAACA |
| | | | TAAAAGCACATAAAAGAATAGTAATATTTAAAATGCATAATACAGAT |
| | | | CAAATATAGGTAAAGGGTAAATATG |
| LNPEP | rs2549782 | 126 | AGAAGAAAATTGTACAGAGAGAAAAGGGTAGCAAAGAGAGAAGAGAG |
| Region | | | ATCCTAACTAATAAAAAAAAAGTTAGTAACTATTGTATTTTTTGCTA |
| | | | AAGTTAATAATTTTTATTTGTTTAACTTCTAATAATATTGAGTTTTT |
| | | | ACCTCCTAGTGGTTTGGCAACCTGGTCACAATGGAATGGTGGAATGA |
| | | | TATTTGGCTTAAKGAGGGTTTTGCAAAATACATGGAACTTATCGCTG |
| | | | TTAATGCTACATATCCAGAGCTGCAATTTGTAAGTTCACAATTCTGT |
| | | | GTATCATACTATATGGTGTAAAGAATCATCAATTCACTATTAAAATT |
| | | | TCAAGTGAATGTTAAACAGAAAAACTACATAATGTTGTGGTTTTTGA |
| | | | ACATATGGCATTTTGTTTGATACAC |
| LNPEP | rs2549783 | 127 | TTGAACATATGGCATTTTGTTTGATACACGAAACAGATCACAGAACT |
| Region | | | GGATGAAACATTGAAGGTTTTAGAAAACAATCAACATAAATCTGTCA |
| | | | CCCCAAAGTCTGTAAAGAGAGAAGGCAAACTAATACAAATGTAGAAC |
| | | | TGTGTATGTGGGTTGAGAGGAAAGAACATCACTGTTTGTATACATCT |
| | | | GCACAGGAATGCYCATGGCTACTAGGTACTATAGAGAGTTCTCGGCA |
| | | | TGTAACCTGAAATCAACATCATTGGGGGTGTACTTTTCTAGAATTTC |
| | | | CAAAGGGTCTGCTGGCTTCCCCAAAATGTGTGAAGGGATTTCTTCAG |
| | | | GTACGTCTTTTTTAAAAACGAATATAGTGGTCTGAACACTACACAAA |
| | | | TTTGCATATCAAGGTGGTTAGTAAT |
| LNPEP | rs2549784 | 128 | GTGGGTTGAGAGGAAAGAACATCACTGTTTGTATACATCTGCACAGG |
| Region | | | AATGCTCATGGCTACTAGGTACTATAGAGAGTTCTCGGCATGTAACC |
| | | | TGAAATCAACATCATTGGGGGTGTACTTTTCTAGAATTTCCAAAGGG |
| | | | TCTGCTGGCTTCCCCAAAATGTGTGAAGGGATTTCTTCAGGTACGTC |
| | | | TTTTTTAAAAACKAATATAGTGGTCTGAACACTACACAAATTTGCAT |
| | | | ATCAAGGTGGTTAGTAATGGTTAATTAGGTCTTTGGGATATATATTT |
| | | | AGTCCAGTACACTATAGACAGACATGGATTCAAAGTTAGGCTCTTCT |
| | | | TGTTACTGTGTATGTACTGAATTTGTGGCTTAATGCCAAAGCTTTAT |
| | | | ATATTCTTATTTGTAAAATGCATAT |
| LNPEP | rs2549785 | 129 | TTTTTGAATGTGTGTTTTGAAGTAATTACAAAAGATTCATTGAATTC |
| Region | | | ATCCCGCCCTATCTCCAAACCAGCGGAAACCCCGACTCAAATACAGG |
| | | | AAATGTTTGATGAAGTTTCCTATAACAAGGTAGTAAATATCAGGTGC |
| | | | AGGTGGAAGCTCTGCTTTCAGAAGTGTAATAATGACTATAGAATCAG |
| | | | CAGTTTAAGTCAYTGGTGCCAGTTCCTGCTACTTGTTTCACTTTTTA |
| | | | TTTGTTCACTTTTCAGACATCACACATGTTCCGTAAAATTTAAGCTT |
| | | | CCTTTAAAAACATACCATACTACCATTTTCTTTATCTCTTTTTTCAA |
| | | | CTCTTTTGTTTTTTTTTAAAGGGAGCTTGTATTTTGAATATGCTCAA |
| | | | GGATTTTCTGGGTGAGGAGAAATTC |
| LNPEP | rs2549787 | 130 | AATGGCCTGTTTCATTCACTTAGTGTGGACTCTCTAAGTATTTGAGC |
| Region | | | TTTCGACCCCACATTTAAAATGTATTTATGTTATTAGCTGCTACACC |
| | | | AAATACCGGTGTAATCTCTTAAGGAAAAGTACAAATATAGTCTTAAC |
| | | | TTTTATAATTTTAAATGGGTTGTTATGAAATCTATTCATTACTTACA |
| | | | CTGGAGAAATTARTATGACGTTTGTAGGGAAATGAAGTAATAAATGG |
| | | | CTGTTTGCATTCATTCCTCTCTAGACTTTCCTGGAAGGACAAAGTGT |
| | | | TTTCTTTCTTTTTCTATGTATCATGAACAGAATAATGTGTGGAATGA |
| | | | ATCAGGTCACAAGATCACCAATGGCTGGAGGGAATTGAGGACCAGGA |
| | | | AGTATATTGTGCATTGCAAGCTTTC |
| LNPEP | rs2549788 | 131 | AGAAAGAAAAGAGGAAGTGAGGCTTAATCCTCAGTCCCGTGGGTCTC |
| Region | | | AGGAACATCTTGGAGGCATAGCTCATTGGAGGCCAAAATTAGGAAAA |
| | | | ACACCATCCTACATATGAGTGAATGCATGAAAGGTGAGCTAAGAAGC |
| | | | AAGCTATTGCTAGTTGAAGGAGGTTTGCCTGCTCAAAGGAGAAGCTC |
| | | | TGAGTAGATTGARGAGAAGATAGATTAACAATGCAAGACTTGGAAAA |
| | | | AATGGAGAATATTGACAATTCAGCAAATTAATCTTTTAGGTAGTTGT |
| | | | GAAATCTTTTTTGCTGTTTCTAGCCTATCCACTTAGATTGTCTAAAT |
| | | | TTAGTAGGAGGAAATTTGCAGTTATTGATGCATTGGTGGAAAACTAA |
| | | | TCATCTTTTCTTCACTAAGTAGACA |
| LNPEP | rs2549789 | 132 | AAGCGATCCTCCCACCTCAGCCTCCTGAGTAGCTGGGACTACAGGCA |
| Region | | | CACACCACCATGCCCAACCAATTTTTAAATTTTTTGGCAGAGATGGC |
| | | | GTCTGCCTATGTTGTCCAGGCTGGTCTCAAACTTCTGGGCTCAAGCA |
| | | | ATCCTCCTGCCTCGGCTTCCCCAATTGCTGGGATTACAGGTGTGAGC |
| | | | CACTGCACCCAGMATGGAGAGAGAATTTGATGCAAGAATTGATATTT |
| | | | ATTTTAGTTCGGTTTTCATACATTTTAAATGTAATTTAAAGACAGGG |
| | | | GTCTTGGATAAGTTGAGTGGAATTGAAATGACAACTTCAATTTGCCT |
| | | | ATAGAAAAAGCTATATTTGTTTCTTTTAGTCCCACACCTTAAAGAGA |
| | | | AAACCCCACATTGGGCGCAGTGGCT |
| LNPEP | rs2549790 | 133 | TCCTCCTGCCTCGGCTTCCCCAATTGCTGGGATTACAGGTGTGAGCC |
| Region | | | ACTGCACCCAGAATGGAGAGAGAATTTGATGCAAGAATTGATATTTA |
| | | | TTTTAGTTCGGTTTTCATACATTTTAAATGTAATTTAAAGACAGGGG |
| | | | TCTTGGATAAGTTGAGTGGAATTGAAATGACAACTTCAATTTGCCTA |
| | | | TAGAAAAAGCTAYATTTGTTTCTTTTAGTCCCACACCTTAAAGAGAA |
| | | | AACCCCACATTGGGCGCAGTGGCTCACGCCTGTAATCCCAGTACTTC |
| | | | GTGAGGCCAAGGCGGGTGGATCACCTGAGGTCAGGAGTTCAAGACCA |
| | | | GCCTGGCCAACATGTTGAAACCCCGTCTCTACTAAAATTATAAAAAT |
| | | | TAGCTGGGCATGGTGGTGTGTGCCT |
| LNPEP | rs2549791 | 134 | GATTACAGGTGTGAGCCACTGCACCCAGAATGGAGAGAGAATTTGAT |
| Region | | | GCAAGAATTGATATTTATTTTAGTTCGGTTTTCATACATTTTAATG |
| | | | TAATTTAAAGACAGGGGTCTTGGATAAGTTGAGTGGAATTGAAATGA |
| | | | CAACTTCAATTTGCCTATAGAAAAAGCTATATTTGTTTCTTTTAGTC |
| | | | CCACACCTTAAARAGAAAACCCCACATTGGGCGCAGTGGCTCACGCC |
| | | | TGTAATCCCAGTACTTCGTGAGGCCAAGGCGGGTGGATCACCTGAGG |
| | | | TCAGGAGTTCAAGACCAGCCTGGCCAACATGTTGAAACCCCGTCTCT |
| | | | ACTAAAATTATAAAAATTAGCTGGGCATGGTGGTGTGTGCCTTCCCA |
| | | | GCTACTTGGGAGGCTGAGGCAGGAG |
| LNPEP | rs2549794 | 135 | TATTAACCTTTTGCTATGTGGTAGACATTATTCTAAATGCTTTTTAA |
| Region | | | AATATTAGGTCATTAATCCTCACAACAAGCCTATGAGGAAGATAGTA |
| | | | TTATTATCCCCATTGTATAAAGAAGGCAATAGGTCCAGAGAGGTTTG |
| | | | GTAATTTGTCCAAGATTACTCAGCTAGTCAGTGGCAGAGCTAAGATT |
| | | | TAAACTGAATGGYCCAGCTATAATGACCTACTTCTGTTATTGTTCTC |
| | | | TATTTCATATTTGTTTTAATGTTAAAAATATTAAAAACTATAAGATA |
| | | | TGCACAAACCACTGACATTTGTTTTATACTTCAGTGCAGGGAGACTG |
| | | | ACCCTAGACAAAGCTCTTGACATGACTTACTACCTCCAACATGAAAC |
| | | | AAGCAGCCCCGCACTTCTCGAAGGT |
| LNPEP | rs2549795 | 136 | TAGTATTATTATCCCCATTGTATAAAGAAGGCAATAGGTCCAGAGAG |
| Region | | | GTTTGGTAATTTGTCCAAGATTACTCAGCTAGTCAGTGGCAGAGCTA |
| | | | AGATTTAAACTGAATGGCCCAGCTATAATGACCTACTTCTGTTATTG |
| | | | TTCTCTATTTCATATTTGTTTTAATGTTAAAAATATTAAAAACTATA |
| | | | AGATATGCACAARCCACTGACATTTGTTTTATACTTCAGTGCAGGGA |
| | | | GACTGACCCTAGACAAAGCTCTTGACATGACTTACTACCTCCAACAT |
| | | | GAAACAAGCAGCCCCGCACTTCTCGAAGGTCTGAGTTACTTGGAATC |
| | | | GTTTTACCACATGATGGACAGAAGGAATATTTCAGATATCTCTGAAA |
| | | | ACCTCAAGGTTTGTGTTGCTTTTAG |
| LNPEP | rs2549796 | 137 | ATAAGAGAAATACGAAGATACACTGTTTGGGGAAAGATTGGGAAAGA |
| Region | | | TGCAGAAAGTTTAGAGTTGAGCCCTTTAGATGGGCAAGAACTGTGTT |
| | | | AAGGACTAAATTTAGCCTCTCTGTTAACCATCTCATATTTTCTGCAG |
| | | | CGTTACCTTCTTCAGTATTTTAAGCCAGTGATTGACAGGCAAAGCTG |
| | | | GAGTGACAAGGGYTCAGTCTGGGACAGGATGCTCCGCTCGGCTCTCT |
| | | | TGAAGCTGGCCTGTGACCTGAACCATGCTCCTTGCATCCAGAAAGCT |
| | | | GCTGAACTCTTCTCCCAGTGGATGGAATCCAGTGGAAAATTAAAGTA |
| | | | GATGTAGACTTCTGTCCTACCCTTTGTTCTTTTCTCTTTGATGTAAA |
| | | | AGTCTTTGATCAAGCAAGACATTAG |
| LNPEP | rs2549797 | 138 | ACAGGCAAAGCTGGAGTGACAAGGGCTCAGTCTGGGACAGGATGCTC |
| Region | | | CGCTCGGCTCTCTTGAAGCTGGCCTGTGACCTGAACCATGCTCCTTG |
| | | | CATCCAGAAAGCTGCTGAACTCTTCTCCCAGTGGATGGAATCCAGTG |
| | | | GAAAATTAAAGTAGATGTAGACTTCTGTCCTACCCTTTGTTCTTTTC |
| | | | TCTTTGATGTAARAGTCTTTGATCAAGCAAGACATTAGGTCTAAAAC |
| | | | CTTTTAGTGAGGATAGAAAAAAAAACATGCTGGGCATTACAAACCCT |
| | | | GTTTCATGCTCTCACATTGTAAGTGCTATGTATGGAGACTAGTACAA |
| | | | TCTCTACCATGAAATGTAATGGTGTCCATTATTCCGGTGCTGGCTGG |
| | | | ATGCAGTGGCCCACGCCTATAATCC |
| LNPEP | rs2549798 | 139 | CCCACGCCTATAATCCCAGCACTTTGGGAGGCCAAGGAGGGTGAATC |
| Region | | | ACTGAGGTCAGGAGTTCAAGACCAGCCTGGCCAACATAGTGAAGCCC |
| | | | TGTCTGCACTAAAAATGCAAAAATTAGCCAAGTGTGGTGGTGCACGC |
| | | | TTGTAATCCCAGCTACTTCGGAGGCTGAGGTGGGAGAATTGCTTGAA |
| | | | CCTGGGAAGCAGMAGTTGCCGTGAGCCAAGATCACTTCACTGCACTG |
| | | | CAGTCTGGGCAACAGAGAAAGGCCCTGTCTCAAAAAAAAAAAAAAAA |
| | | | CTTTTCCTGTGCCAAATTATTATAAGATGGTATCATAACTTCTCTCG |
| | | | CTATAACTAAATCTGTGAGCTTTTTGAAATCCTTTCTTGAATTCTTC |
| | | | TTTTTAAAAAAGTAATTCAAGTTTT |
| LNPEP | rs2549799 | 140 | CTATAATCCCAGCACTTTGGGAGGCCAAGGAGGGTGAATCACTGAGG |
| Region | | | TCAGGAGTTCAAGACCAGCCTGGCCAACATAGTGAAGCCCTGTCTGC |
| | | | ACTAAAAATGCAAAAATTAGCCAAGTGTGGTGGTGCACGCTTGTAAT |
| | | | CCCAGCTACTTCGGAGGCTGAGGTGGGAGAATTGCTTGAACCTGGGA |
| | | | AGCAGAAGTTGCMGTGAGCCAAGATCACTTCACTGCACTGCAGTCTG |
| | | | GGCAACAGAGAAAGGCCCTGTCTCAAAAAAAAAAAAAAAACTTTTCC |
| | | | TGTGCCAAATTATTATAAGATGGTATCATAACTTCTCTCGCTATAAC |
| | | | TAAATCTGTGAGCTTTTTGAAATCCTTTCTTGAATTCTTCTTTTTAA |
| | | | AAAAGTAATTCAAGTTTTCTTCTTT |
| LNPEP | rs2549800 | 141 | ATCAGCCCCCTCCCACCATCCCTTCTTGGCTCTCTCCTCAGCCCAGA |
| Region | | | AGAATCCCAAGATGCCTTGTATAGTGCATCACACATATGTGGCCTCA |
| | | | TTTGTTAATGCAGTTTTGTACTGGGGGAAAATCTCAGGAATTTTGGT |
| | | | AAGAGCTACTCCTCACCCCTAGAGCCCCTGTGGAGGTGGCACAGTGG |
| | | | AGACCTTGGTTCMGGTGAAAGAAACCTAGTCAGGAGTGTTTGGGGAG |
| | | | CCCTCCCTCCAAATTGTTTTGGTTCTGAGTCTTTTCTGGGGTTTCAA |
| | | | CTTTGGGGAGAACTGGAGCTCATTTCAATATGTTCCTGGATCTAAAA |
| | | | TATCCCTCAGAAATAACCAATAATGATTAGATTTTGTTGGAATGGAA |
| | | | TGTATAAGACAGCATTGCTTTCTGT |
| LNPEP | rs2549801 | 142 | CTCACACAGCTTTGCGTAAGCAAAAAGCACATTTCCACTCCTCTCCC |
| Region | | | AAATGCTCAAGGAGTTGACGTCCACATGAGACCAAATAGAAACTGCT |
| | | | TTAATATGTATGTTTGTGTATGTTTCCTTTAAAACTCTACTTGAGCC |
| | | | ATTGATTTGTCTGTTTTCATGATTGTCATTTTCCTCCTGAAATGATC |
| | | | AGCCTTAATCTAMAATGCTGTGGATTTCATTTTAAACAGGAAATGAT |
| | | | GCCCCTGCCCAACCATGTGCAGAGCCTAGACTTGGAACCATATTTTT |
| | | | TTCTGAGCTGTAACTGTGCTCTGCTTTATCAACATGAATCACTTTGA |
| | | | CCTGTAGTACAAGTTTTCCATATATAAAACATTGGGTTGAAGGATGC |
| | | | ATTCTGTGGCCGTGAATGAGAGTTC |
| LNPEP | rs2617434 | 143 | TGATTTTCTGGCGCAGTGCGGGTGTCTCGGCGTCCGGGATCGGGCGG |
| Region | | | GTCGCAGTAGGGCTCCACATTTGTTGAGTGACTGAACACCGTTCCCG |
| | | | GCCGGGGAGAGCGCCGCAGCCGGGTCCACTTCAGGTAGGGGCTGGGC |
| | | | TTTCCCGGCCCCGCCTAGGCCCCGCCCCCAGCGCGAACCCGCTCCCA |
| | | | CCTCGCCTGTCCRCGGAGCAGCAGGGGGTTTGACTGTGCTTTTCCCT |
| | | | CTTGCTTCCCTCGCTCTTTCTGCAGCTGCCACGAAAACCCGGAACGG |
| | | | CGGAGCGGCGCCGCCCCTCGCGGCACCTCCCTGGCAGCCCTTGGAGG |
| | | | CCGCGCTGGGCATGCTCAGTCAGCTGGGCCGCCTCAGCTCTCGGAGT |
| | | | AGGAAGCTCGGGCGCTCCGGCTGTA |
| LNPEP | rs2617436 | 144 | CAGTAAGGAAGTAAGTAGAGGCAGGTGGTAGGGTGGCAGTAAGAATT |
| Region | | | GATTCCCCCAAATTAACTATGCTGTTTGTCCTAATTTTATATGTGTT |
| | | | GTAGCTTTACCCTTCAAAAAGAAAGAAACTTAGTTCTATTTACAAAG |
| | | | GTAGTAAATTCAGTTTGATTTAATTGTGCTTTCAAAAGTAGTGTAAA |
| | | | GGGAAAAGAACCRAACCTTAAAAAAATTCTGTAAGAATATTATAAAC |
| | | | TCAAAATTTATTTCCATGGCTTTTGACATATTGAAAATAAACTGGGG |
| | | | ATAAATACCTACCTTGACCAGCAACCTTTACACCAGTAGCCATAAAA |
| | | | TGAGGCCATTCAGATAATGTTATTGAAAGAGGTGAAGTTCAATGCCA |
| | | | TTCGTAGTAATAATAATATCTGGTA |
| LNPEP | rs2617447 | 145 | TAATTAGTAATGTTTGAAGTTGTATCAAATCAAGAAATGTTTAGAGC |
| Region | | | ACAGAAGAAACCAGAATAATTATCTAATAAAGTTCAAGTAGAGCTTA |
| | | | GGCATTAGCAAAAAAACGCAGCCAAATAAAGTGAAAGGTTATTATTT |
| | | | GGAAAGAACAGTGATATACTAGTTCAGATTCCTTGGGCTACAAAAGA |
| | | | CAAAACTCTGAGYAAAACTAGTTTAAATGATACAGACATTTATCATT |
| | | | TCATATAACAAGTCCACTGATAAGATAATCTTCAATCACAGCTCACC |
| | | | AGCCTCATCAAGAGCCCAACTTCTCTCTCTCCACTCTTCAGTCCTCT |
| | | | ATATGAGCAATTCCCCAAAGCCCAGACTACCATATGGTCAAAAGTTG |
| | | | GCTGCAGCAGGGGAGGAAGAAGGAA |
| LNPEP | rs27289 | 146 | AGGAAGGGCATATAGTAATTAAAATATTTATCATGTGCCATTCTCTG |
| Region | | | CTCTTGCCTTTTTTTCCCCTAATAAAGGAGAAAGAAAGGGGTATTAG |
| | | | AGAAGGGAATGCTTTTGAACAGGAGTGATAAAGTTCAATAGCATGTA |
| | | | TGATGCTAGCCCTCTGGAGCAAACTGTACAGGAAATTTTGTAACTTT |
| | | | GTAGTAAAAACGKGTTTTTTAGTTTCAGAACTTTAGTTTTTTTGTAA |
| | | | AACAGTAGTTGATTTTCGTAGCTCATTGACAAATGGTTTTTAAAAAT |
| | | | CACTGTTAGATTACTTCATCTGGGCTTCTGCCATTTAATATTGCAGT |
| | | | TGCTCACTCTTTTTTGCTACTCAATAGACTGAAAATTGAAGTGTTAA |
| | | | TCTGTTGATGACTATAGTAAATTAAA |
| LNPEP | rs27290 | 147 | CATTGGTTTTGAGGGACATCTCTGATGGCTGGAGCCACCTTATCTGT |
| Region | | | ACTGCTTGCCTCCCCAACCACCTCATGCATTATAGAATCCCAAAGCC |
| | | | AAGGATGACAGTGACCTCATGTAAACATATTCTCTGAATAGAATATT |
| | | | ACCAATTTAGATTGATGATAGGCTTAAAAACACTGACGTGTTCCTTC |
| | | | TCATCTCCCTGGRCATTTCCATTTTGTCTCGTTTTTTATGAAGTGCC |
| | | | CTTCTTACGTCATCCTAGCCATTGCTGCTGTGATTCCTATAAAATGG |
| | | | TTAATTTTAAAAATGTACTCACTGTAAATTCACAATAGACCTTGTCA |
| | | | TAACAAGTTTGAAAAACAAATTCCCATTAAACCAACAAATAAAATTA |
| | | | AAAAAAGAAATCAGATACTCTCTAT |
| LNPEP | rs27291 | 148 | TCCTTCTCATCTCCCTGGGCATTTCCATTTTGTCTCGTTTTTTATGA |
| Region | | | AGTGCCCTTCTTACGTCATCCTAGCCATTGCTGCTGTGATTCCTATA |
| | | | AAATGGTTAATTTTAAAAATGTACTCACTGTAAATTCACAATAGACC |
| | | | TTGTCATAACAAGTTTGAAAAACAAATTCCCATTAAACCAACAAATA |
| | | | AAATTAAAAAAARAAATCAGATACTCTCTATGCTATACTCTCTTTCC |
| | | | TGGCAAATATAACTAATTAATAAATAAAATTAGTACTATTCATCTTT |
| | | | TCTAACCCAAGATATTATACTTTTGCTGAGTTAGTAGCAATTTACAG |
| | | | GAGACTTAGGAATAAAAAAAAATGAGCTATTGTTTATCTTTCTCTTG |
| | | | AAATGCCTCTTTAATCTTGGGCCTC |
| LNPEP | rs27292 | 149 | GATTGGATGATATTAATGAATAACTATTAGTTTTCTTCAGTGTGATA |
| Region | | | AGGTATTATGGTTCTGTAAGAGAATGTTCTGATATCTGTGAGTTGCA |
| | | | TGCCAAAGTATTTATAAATGAAATATCGTATCTGTATATCACTTTTA |
| | | | TATTGTTCAGCTAAAACAAGAAAAACATGTGTGCGTATGTGTGTTAT |
| | | | ACACACTTGAAARTGAAGCAAGTGTCAGAGTGTTAAAAAACTGTTGA |
| | | | ATCTAGATGAACAGTGTATGGGTGTTGTACTATCTTTTTTTGTAGGT |
| | | | TTGAAGTTCTTTAAATAAAAACTTAGGAGAAAAAATAAGCTATAAA |
| | | | CAACTATTCTTCCCTGCAGGTCTCATTTTCTTGCTAATTTGGTTAAT |
| | | | TTGTTATAACATCAAACTAGTTAAT |
| LNPEP | rs27293 | 150 | AAATGAAGCAAGTGTCAGAGTGTTAAAAAACTGTTGAATCTAGATGA |
| Region | | | ACAGTGTATGGGTGTTGTACTATCTTTTTTTGTAGGTTTGAAAGTTC |
| | | | TTTAAATAAAAACTTAGGAGAAAAAATAAGCTATAAACAACTATTCT |
| | | | TCCCTGCAGGTCTCATTTTCTTGCTAATTTGGTTAATTTGTTATAAC |
| | | | ATCAAACTAGTTRATAATGTTAAATATACCTTTAATATTGGATTGAG |
| | | | AAACATTTAAACATTAACTATCAATAAAGAAGTTTATTTTTCTTTCA |
| | | | TTGCTTTATAGGTTTATAGATTGTAAAGTCACAAGGTCAGGAAGTCC |
| | | | TGACATCCTTCCAGCAGTGGTTATAAGTGATACTTTTTGGCAGGAAA |
| | | | ATAACTTCTAGCTGAGTATATGCAG |
| LNPEP | rs27294 | 151 | GTTTGAAAGTTCTTTAAATAAAAACTTAGGAGAAAAAATAAGCTATA |
| Region | | | AACAACTATTCTTCCCTGCAGGTCTCATTTTCTTGCTAATTTGGTTA |
| | | | ATTTGTTATAACATCAAACTAGTTAATAATGTTAAATATACCTTTAA |
| | | | TATTGGATTGAGAAACATTTAAACATTAACTATCAATAAAGAAGTTT |
| | | | ATTTTTCTTTCAKTGCTTTATAGGTTTATAGATTGTAAAGTCACAAG |
| | | | GTCAGGAAGTCCTGACATCCTTCCAGCAGTGGTTATAAGTGATACTT |
| | | | TTTGGCAGGAAAATAACTTCTAGCTGAGTATATGCAGCATAAAGGTT |
| | | | CCCTACTGCACAGAAGTCATTAATTTTTTTCTGAGTTAAcATCACTA |
| | | | AAAGTCCCCCTTAGCTATAGCAGCC |
| LNPEP | rs27296 | 152 | GGTTTATACCATACCCAGTGTTTTGTGACCATCTTTTTAGTGAATGA |
| Region | | | TCAGTCTATGAGATTTTCCATGTCACTTCATGAAGCCTGCTTTATAT |
| | | | ATAAAAAAAAACTAAAGTGTTTTATGGGTTCATAATATTCTGTAGTA |
| | | | TGGCCCTATCATAATTTATTTAATCCATCACCTTTTGTTGGGCTTTT |
| | | | GTTTTTTTCTCTYCTAAAAATACTGCCACAGTCTTGGAGTGGGGCGT |
| | | | GGTTTCTCACTATAAACAGATAGTATAGCATAGTAGATGAGAACTGC |
| | | | TTTTGTTCAGATGTCGGGCTTTGGTATTTATTACTGTGTGACTGTGG |
| | | | GTCAATTAGATAACCTCAGTTTCTTCAACTATAAAATTGAGTTAGGT |
| | | | AGTATTAATAAATACCTACTTTATA |
| LNPEP | rs27298 | 153 | CGGAAATTTTCACATTTGTTAACATAATTCCATAGCATGAATCATTT |
| Region | | | AACAGTAGCATTCCATCTAAGTTCTAATTAAGCCTAGCCTTGCTTGG |
| | | | CACCAGGTTACTTAGCTCAGCGTGGCTACAGACTATTTCATAGCAAC |
| | | | CATTTAGCTATGCATATTGAAAAATACCTCTGTATGGCCGGGCGCGG |
| | | | TGGCTCACACCTKTAATCCCAGCACTTTGGGAGGCCGAGATGGGCGG |
| | | | ATCACGAGGTCAGGAGATCGAGACCATCCTGGCTAACACGGTGAAAC |
| | | | CCCATTTCCACTAAAAATACCAAAAATTAGCCGGGCATGGTGGCGGG |
| | | | TGCCTGTAGTCCCAGCTACTTGGGAGGCTGAGGCAAGAGAATGGTGT |
| | | | GAATCTGGGAGGCAGAACTTGCAGT |
| LNPEP | rs27299 | 154 | ATCGAGACCATCCTGGCTAACACGGTGAAACCCCATTTCCACTAAAA |
| Region | | | ATACCAAAAATTAGCCGGGCATGGTGGCGGGTGCCTGTAGTCCCAGC |
| | | | TACTTGGGAGGCTGAGGCAAGAGAATGGTGTGAATCTGGGAGGCAGA |
| | | | ACTTGCAGTGAGCCGAGATTGTGCCACTGCACTCCAGCCTAGGCAAC |
| | | | AGGGCGAGACTCYGTCTCAAAAAAAAAAAACAAAAAGGAAAATACCT |
| | | | CTGCATTGCCAAGGCATCAGTTAAGAACTCACATTCAGCTAGATGCA |
| | | | GATGTAGGTTTTTTGCTTCTTTCTCTCTTTTAAATCAATAATGGCAT |
| | | | TTCTGGGTGTACAGTGTGATCTTCGACAATGTTAAGCGGATTAATTG |
| | | | TCTGCATGTTCTGAACTCTTCCCTT |
| LNPEP | rs27300 | 155 | AATGTTAAGCGGATTAATTGTCTGCATGTTCTGAACTCTTCCCTTTT |
| Region | | | TCTGCCCACCTTTCCTTCCCACCGACAATAAGTATGCATAGGGCTAC |
| | | | CCCGGTTTCCTCAGTTGCAGTTCCGGGAGGAGGATTCCACTCTGGCT |
| | | | TTGGCATTAAAGACTTTTCCTTGCACTCAGGAACAACGCTTTACCAG |
| | | | CAGCTGCCTAATYTTTTTGCTCTTGTTTTTGTGTTTCTTCAGGTTCC |
| | | | CTCTGGGGTCCTATACCATACAAAATATTGTTGCTGGATCAACTTAC |
| | | | CTGTTTTCAACAAAGACACATTTATCTGAGGTTGGTTTTATAAAATG |
| | | | ATAATACAGAGACTGGGCAACCCTCCGCACACCCGGACCAGGCTGCT |
| | | | CAGTTTTAGTGAGATGGTGGATTTT |
| LNPEP | rs27302 | 156 | GTTAAAACTTTGAGTGGATGCATAGGGCGGATAGCTAACAGTCACGG |
| Region | | | GAGCTCCATCAGGACCATTATTTACTTTTTGGACTAAAGCAGTTCTT |
| | | | GTAAACACTCAGGTCACCTAAGTAGCCAACTGATGCAGTAGTCATAC |
| | | | AGTACCTAAATCAGTGTGAGAAATGTCATACGTGTCGTATGCCAGTG |
| | | | AAACCAAGGAACRCTGTCTTACTTTGAAGGTGAGACATTGGATGTTA |
| | | | TCAGGGAAATACCCCTTGCGTTGATTCACATATAAGTAGGAGTATGA |
| | | | GTGCACCTTTTTAGAGGCACACTGCCACGGTTACATTCCTGGTCAGG |
| | | | TCTACAAAAGGAGTTTCTTGGTTCTGTCTGCATGAGTAGCCTTGAGG |
| | | | AAGAACTGAGAATTTCTTAGGCTTC |
| LNPEP | rs27305 | 157 | GAGATACACAGTTTGACCACTTGGTGGTGCCCAGAATGTGTAAAAAG |
| Region | | | GTCTAATACATGTTCTAGGGGAGCTCATCTGCTGAGCTCTTAAAGAA |
| | | | TTATTTCTGTTTAAGAGTTACATTTTATTTAAAACCGACCTCAGGTA |
| | | | AGGGAAATGTATATTTTCTTAGTAGAAATTCTAGACTATATATAAGA |
| | | | AATCAGCGTAAGRACCAGTTTTTGTTCTTTCTCTTTTTAAATTTCAA |
| | | | GTTTCATTTAAAAAAATATATGCTAACCTTTTTAGAATATTCATCTT |
| | | | GGATACTCAGAGTTGGCATTTGTTTACTTTGGTAATAGATTCTTAAT |
| | | | TTTCCCATATGCTGTTGTTTAGGAAATGCTAATTTTAATGTGGCCAG |
| | | | GTTATAATTGTATCTTTAAATTTAA |
| LNPEP | rs27306 | 158 | GTGATAACGGTGTCATTCACATTTATGTTGTGGGGAGGGATGAATGT |
| Region | | | TATGGCTGTCAGACAAGATAGAGAAGAAAATACACAAAATGTGTGAG |
| | | | ATACAGTCTATGTCATCAAGTAGCTGAAAGTTCAGATGGTTGGTACT |
| | | | TGTGGAGCAATAAGAGAGGTAATATGTGCTGAGTGGGACAGAATTTT |
| | | | TGCCTAGGATAAKGAAGAGAAACTTTTGGGAGGTAAATGGGAATTGA |
| | | | GTTGGCTTAAATAATTAAAATATAGGTAAAGAGGAATGTGAAGTATA |
| | | | GGGTAGGGCAGGAATGGAACAATGAGGTCTGCAAAGAAAATGAAGTA |
| | | | CTAATGGGCAAGTGATGTTTTTTCACAGAGTCAGTGTTTGACCACAG |
| | | | TGGAAGCCACACGTGAAGAGGGAAA |
| LNPEP | rs27307 | 159 | GTGCCTACTATGTAGTGGGCATTGTTTTTGTCATTGGAGTTGAGTTT |
| Region | | | TCACTTTTTCCTCTCAGCTTACTGCATAGATGAGGAAATAAATGTGT |
| | | | AACAGATACAGGATGCCATATAAATTGTATTGAAGATGGAGAAATGT |
| | | | GATTTCTATTTCAAACCGGGAAGGTCTTCACAGAGGAGGTGGTGCTT |
| | | | ATTTCAGGATTGSGAATGATGACAAGCACATCAATGGATATAGTGTG |
| | | | GCCTTAATATTATGCTCCCTGCCCTATACATTTGTTAGTTCCAAGCA |
| | | | TTTGAAGTGACTCTGCAGGAAGAAGAGAGATTCTGGTTACTTCACAA |
| | | | CACAATATACTAAAATAAGAAATTAATGACCTACCTAGCAGGGAGAC |
| | | | TTTTGAGAGCCTTGTCAATTTATTG |
| LNPEP | rs27397 | 160 | ACTTTCTAGCTGTTAGTCTTTTGCCCAGAAGATATACCTTCCCTACT |
| Region | | | CTCTGAATTCTCTTTGAGAACTTATTACCTAACAGTTGTTCCCAAAT |
| | | | CATTGTTTTTTTCCTCTGATCATATAGTAGTATTTCAATTGAAAGCC |
| | | | ACATTGTTTACTTTATACTTACTGTCTTAATCTGTTGGCATTTAGAA |
| | | | CATATTTCTGCCKTTCTCATTGACTGCAACTTTTGCATCATGGTGTT |
| | | | TTTCCATCCCAAACTAGTTCCTAACATTATCCTCAGGTTTTTCAGCA |
| | | | CCCACATCAAATTATGCATTGGCCTCTTCACTTCAATACTGCTTCGA |
| | | | CACCCAAAAGCACCTTGGTTTTAGGCAAGCTTATTTTCTTCTAATCC |
| | | | TCTGGTTCAGAAATAAATAGGTCAG |
| LNPEP | rs27436 | 161 | TGCACCAGTGCACTCCCGCCTGACAACAGAGTGAAACTCCATCTCAA |
| Region | | | AAAAAAATAGGTCATATAGATAAGATGTCTTTTGGGGGATCTCCTCT |
| | | | AGGTCTGTTATTTCTGAAGCCACCCATCACCATTTGGGAGTATTTCT |
| | | | CTGTTATTTCCTCTTTAGGACTTAGAAATCATCTTCCTCTGAAACAG |
| | | | GTTTTAAAATAAYATCTTAGAAAAATGTTATTGTAATTCTCAAAGGT |
| | | | GTTTTGTTTTATGCAGTAACCTCGTCCTTTCGCTGCTGATTTGAGAT |
| | | | AAGCCCAAGACCACACTGACCAAATTACATATTTTACAACTACTTTT |
| | | | CATTTCAAGGATTTTTTTAGATACATTTTTTAAGGAGAATCTCCTAT |
| | | | TATTTTTTTCCTTTTTCTTTTCTTT |
| LNPEP | rs27613 | 162 | GTATTCTATTATTTTCTTTCTTTTTCCTCACTTTTTTTTTTAATAGG |
| Region | | | GATCTTCTCTCTTGTTGATGTTGAAAACTTACCTTAGTGAAGATGTG |
| | | | TTTCAACATGCTGTTGTCCTTTACCTGCATAATCACAGCTATGCATC |
| | | | TATTCAAAGTGATGATCTGTGGGATAGTTTTAATGAGGTAAGTGACC |
| | | | TGGGTAATTTATKTAGCTCTTACTGTAAAAAGAGAGGAGTTCGTCTA |
| | | | TTTATACTTTTTAGCATGTGTGTAAGTTAATCTGTGGTACAAAGCAT |
| | | | AGTTATTTAAGAAAGGGGGGGATGGAGCTTGCTATATAAATATTTAT |
| | | | GAATGGAGCACTAAATTTTATGTCAAGAAATGGGAGTGCTGTTCTTA |
| | | | GTTGTTGGAAAAGACGTGTGTGGGC |
| LNPEP | rs2762 | 163 | ATTCTATAAGAGAAAAGACACCAATTTTAAAACTTGAGAAAGTACTT |
| Region | | | TAATTCTGTAGGCAAAGGTTCAGCAAATCAGCTAGCACTAATCTTGA |
| | | | CCAAATGGGTGAGTCAGCCTCATCACAGAGATTTTTTTTTTAATTTA |
| | | | GATGAAATTTCACATTTAAAAACATGGTAACTCCAAGCATTCTTCCA |
| | | | AAAACAAAGAATRAACATTGGAATAGTCACTTACAAGGACTTAACGA |
| | | | CTTGTATTAAACATATTTTACACTAAAGTACTAGATGGTCTCTAGTT |
| | | | CATTTTAGCTCAAACCTTCCTTAATTCTTCGAGTAACTTCACCAGAC |
| | | | TATTCTTTAAGTCTTAGTAATTACAAATAGAACCTGAATGAGAAAGA |
| | | | AAATTCAGAATTAAAGTTGTTATCA |
| LNPEP | rs27621 | 164 | TAAAGTATTAGCCAGCCTCTGACTTAAGCAAATAGAGAAAAATCACT |
| Region | | | GTTTTTAAACTATGCTAGTAATACACTAAGAATGCCCATGATAAAGA |
| | | | ATTTAAACAGTACCTAAGAATATAGAGTAAAATATGAAAGTATTTCT |
| | | | CATGATCCTTCACTTCCATTCTCGTTTTCTCTGTTAACAACAGTGTC |
| | | | AGTCCTGGTCTTYGTATTCATCTGTGGGAATGGATATTTGTACATAT |
| | | | GTACGTACATACATACACACATACCTACATATTTAACTGCATTTTAA |
| | | | AAACCATATTAGGTTTATACCATACCCAGTGTTTTGTGACCATCTTT |
| | | | TTAGTGAATGATCAGTCTATGAGATTTTCCATGTCACTTCATGAAGC |
| | | | CTGCTTTATATATAAAAAAAAACTA |
| LNPEP | rs27659 | 165 | TTGTACTTAGTCAAAATCATTATGTATATATGATTTCTGTATCTTGC |
| Region | | | CTTTTTTAACTTAACATATATAGTAATTGACTTAACCTACTTGATTC |
| | | | ACCATTTGTTGTTATAAAATATTGCCTTAATTAAATATTTCAAATCT |
| | | | TAAAGGTTTTTCCATTATATGAAATTTTTTAAGGAATGCTTTTCAAA |
| | | | AATGAAGACTGCRGTACCTTTTGTGATTTGGTACATATAACCAAATC |
| | | | GCCCTTTTTATTGTGTAGTTACAGTTGATAATGCCACCTGAAATACA |
| | | | TGAATGTGCCAGTTTCATTGCAGTTTTACCATAGTGGAGTGTTAAAG |
| | | | CAGCAACAATCTAATTATTTAAAATCCTAGTGGTAGTTGGTAATGTC |
| | | | ATCATATCTTTGATAGCATAGCTGG |
| LNPEP | rs27712 | 166 | TTTTAAGTTAGAAAAATGTATCTGTGTGGGAGTAAAAAGATTTCCTT |
| Region | | | TTTAAAATCATTTCAGATATCACCATACTTGATTGGGAACTCCATGT |
| | | | AGATACCTTGAATATTAAAGTACTTCTTTCTACTGCTTTCAAGATAG |
| | | | CAGCACAGCCATCACTAAGTTAAAGCTTATTTTAAAAGCTGGGGTTA |
| | | | CCTGAGGTTTATYGTCCTTTTCTTCTTTTGAAATTCTTCTGTGTGAA |
| | | | ACTTACAGGTTCAGGCATTCTTTGAAAATCAGTCAGAGGCAACCTTC |
| | | | CGGCTTCGTTGTGTCCAGGAGGCTTTGGAAGTCATTCAGTTGAATAT |
| | | | CCAGTGGATGGAGAAGAACCTCAAAAGTCTCACATGGTGGCTGTAGC |
| | | | ATGCACAACCGCACCTCATTTTGTT |
| LNPEP | rs27747 | 167 | TACCGTGCATTATGGAGAGAAGATTCAAGCATCAGATGAAGAGTTGG |
| Region | | | GGTGGAGGATTTGGATAACGTTTTGAGGTCTTTCCCAGCTCTGAGAT |
| | | | CTTAATAAAGGCAGTAGACTGTGTTTTCTCCCTGCACCCCATTTACT |
| | | | GCTATAGTTCTACCATGAAACTTATCACACTGAATTGTAATGCATAT |
| | | | AGTTATTGCTCTRTACTTCGTAGTAGCCTGTGAGTTCTCAGAGGACA |
| | | | GAGGCTCTCATTCCTTTTCCGCTCCCTAGTGTCCAGCCAGTCGCCTG |
| | | | CCTGGTTCTTTGTGAGTACTCTGTGAATATTAAATTGAACTGATGTA |
| | | | TCCATAGACACACTACTAGGAAGATAGCAGTCACTGAATTAAACTTT |
| | | | TTCTCAACCCTAAATTGTGTACTCA |
| LNPEP | rs27993 | 168 | TCAATGAGTATTGCCATTGTTCTTCACTGATTTTTTTTTTAAATAAG |
| Region | | | ATTTCAAGCATGTGATTTTTTTTCTCACATTCTTCATTTGTTCCTAT |
| | | | TTGACAGTTATGAGTAGGATTTGAATTTCTTTTGTTCTCCAGTCATT |
| | | | TGGAATGGTTTTCTATCATAATGCTATTGAGAAGGTAAGGCCAGTGA |
| | | | AGACACCACATARCAATGCAGTTAGGTATGTCAAGTGGGATCCCCTG |
| | | | CATTGCTTGTCCGTTCCTTGCATCGTCAGCGTAGCAAGTATTTTTCT |
| | | | ACTTCATGTCCTCCCAGTGACTCAAAAGCTTTACCACTTACACATTC |
| | | | CACAAGGTGTCTGTTCTGTGTTTCATTGCTTTTGAAACAAACACAAG |
| | | | CGAGTTGACATGTTATACAAACCTT |
| LNPEP | rs27997 | 169 | AAAAGAGAGGAGTTCGTCTATTTATACTTTTTAGCATGTGTGTAAGT |
| Region | | | TAATCTGTGGTACAAAGCATAGTTATTTAAGAAAGGGGGGGATGGAG |
| | | | CTTGCTATATAAATATTTATGAATGGAGCACTAAATTTTATGTCAAG |
| | | | AAATGGGAGTGCTGTTCTTAGTTGTTGGAAAAGACGTGTGTGGGCTT |
| | | | GGGTAGCCAGTTKTTTTTTTTTTTCCTGTACCTTAACTTCTATTCCT |
| | | | ATTTTGTAGGAAAGTTGTCTTCTCCGTATTAATGAATATTACTATAT |
| | | | TTTCATTATTTGACTTTTTTTCCAGAAATCTCTTTTCCTATCCTTAC |
| | | | CCTTTTAGTTTTTCTGCCTCTTTTGAATGATTCTGTACTCTCCTCTA |
| | | | TGAATCTCTTGCCTTTGTGACTGTT |
| LNPEP | rs2910686 | 170 | GTCTCGATCTCCTGACCTCGTGATCCACCCACTTTGCCCTCCCGAAG |
| Region | | | TGCTGGGATTACAGGCGTGAGCCACCAGCCTATCTTTTTTTTTTTTT |
| | | | TAAAGCATTATAGTCTTTGCACCTTCTTTTCACAATAAATCTTGAAT |
| | | | TTATTTACCCTTTAGGCAAATTCAGAATTCCTGAACTTAAATCCCAG |
| | | | CTCACCATTTACYCTATGACTTGGGTAAATCATTTAACTTCTTTAGC |
| | | | CACATTGTGGTCACTTGTAAGATGAGGATTTATAATTTTTGTCTTAC |
| | | | TTTACCTATTGTTTGAAATAAAGTGAACAATTATGCAGAAAAGTAG |
| | | | AAAATAACCTTTTAGAGGTTGGCAGAGAAATGCCTATACCTGTGTGT |
| | | | ATGTAATTTGCAAGCTCTTTTGAAA |
| LNPEP | rs2910688 | 171 | TCAAAATAAATGTTCAAATTGTGGAATTGAAAGTAAACTTAGATATA |
| Region | | | ATCTGATTTGAAGTTCTCATTTTAGTTTTTAAAAAAGTTGTTTGTCC |
| | | | AATGCCATATAGCAAGTAAATGGAAGGGACAGGATTAAAACCTGACA |
| | | | CTTGGCCAGGCACAGTGGCTCACACCTGTAATCCCACCACTTTGGGA |
| | | | GGCCAAGGCAGGYAGATCATGGGGTCAGGAGTTCGAGACCAGCCTGG |
| | | | CCAATATGGTGAAACCCTGTCTCTAATAAAAATACAAAAATTAGCAG |
| | | | AATGTGGTGGCACGCACCTGTAGTCCCAGCTACTTGGGAGGCTGAGG |
| | | | CAGAAGAATCACTTGGACCCAGGAGGCGGAGGTTGCAGTGACCTGAG |
| | | | ATCACACCACTGCACTCCAGCCTGG |
| LNPEP | rs2910787 | 172 | ATTGGATTTTGTTACACGTTCATCCTCTTTTAATGGAATCTTTCCCA |
| Region | | | CTTACACTTTTTCATGTATCCCTATATATGTAGAGAGGTGTATGAGC |
| | | | TTAACAAAAAACAGTTTCAGTAATTTAGGACCACATATCTTTTAGTT |
| | | | AAAATCTTGTCAGTGGTTCCATCTACTGACCTATGCATTTGTAAAGG |
| | | | AAGTGAATTTACYTTATATCTTGTCACTCTAGCCTTCAATACTCATC |
| | | | TATTCCAGTACGTTTTTTTTGTAGTTTCCCTGTTTTCTGTCCAAAGT |
| | | | TGCCACTGGTATGACCTATTTTTGTTGGGCCCTGCTCTCTACCTGTT |
| | | | GATAATTGGTTCATTTGATGAATATCCTATGTTAACCTGTTCAGGTA |
| | | | ACATACTTCTGCAACCCATTTAAAA |
| LNPEP | rs2910789 | 173 | AAGAGAAATGAATAGAAGAACCTAGTTTTGTTGTCATGCTAATATGA |
| Region | | | AATATGGAAACACAGAAGAAATAAAAAAGCAATAAAGTTTTGTCTAA |
| | | | GACAGTATTCTAATTATGAAATAAATGTACAGAAACTGTTCATAACT |
| | | | GTTTGCATGTCTACTAATTTAGTGTAATACTCCTATTAGGAAACAGC |
| | | | AGTATTAACCCTSTCTATGAAAACATTAGGAAATTGAGATTTGAAAG |
| | | | AGTTTAGCCAAGATTACCCCAGATGTTGGGATGGACCTAGATGAGGC |
| | | | CTGTGGTTCTTGGCAGTCGAGGTGAGGTCAGTGCAGCTATGTTTTGT |
| | | | CAATTAGTCACCTCATGACTTGAAAACTGTAGGGCAGCAGTCCCCAA |
| | | | ACTTTTCGGGACCAGGGACCACAGT |
| LNPEP | rs2910792 | 174 | CCTGGTCCCGAAAAGTTTGGGGACTGCTGCCCTACAGTTTTCAAGTC |
| Region | | | ATGAGGTGACTAATTGACAAAACATAGCTGCACTGACCTCACCTCGA |
| | | | CTGCCAAGAACCACAGGCCTCATCTAGGTCCATCCCAACATCTGGGG |
| | | | TAATCTTGGCTAAACTCTTTCAAATCTCAATTTCCTAATGTTTTCAT |
| | | | AGACAGGGTTAAYACTGCTGTTTCCTAATAGGAGTATTACACTAAAT |
| | | | TAGTAGACATGCAAACAGTTATGAACAGTTTCTGTACATTTATTTCA |
| | | | TAATTAGAATACTGTCTTAGACAAAACTTTATTGCTTTTTTATTTCT |
| | | | TCTGTGTTTCCATATTTCATATTAGCATGACAACAAAACTAGGTTCT |
| | | | TCTATTCATTTCTCTTATTTAGGTA |
| LNPEP | rs2927609 | 175 | AGGAGAATGGCGTGAACCTGGGAGGCGGAGCTTGCAGTGAGCCGAGA |
| Region | | | TCGCAAGCCACTGCACTCCAGCCTGGGCAACAGACCAAGACTCCGCC |
| | | | TCAAAAAAAAAAAAAAAAAAAAAAAGATAACTAGAATTACCAACAAT |
| | | | AGTTTTGTTAAAAAGATCATTAAGTACGCTTCCAAACTTTAATATAA |
| | | | TCACTCTTGCATYGTAATACAATATGAAAGAAATAATACAAAAGGGC |
| | | | TCACCTCTCAAGTCTATTTTCATTTTGAATGCTATGAATACACGTAT |
| | | | TTTAAGTATTTTAAGAGTCAGGGGCTTTTTTTTGCTGTTGTTTTTTG |
| | | | TTTTTGTTTTTGTTTTTTGTTTTTTTGAGATGGAGTCTCACTCTGTC |
| | | | ACCCAGGCTGGAGTGCAGTGGTGTG |
| LNPEP | rs3096167 | 176 | CTGGAGTCCAGTGGTGTGATCTCAGCTCATTGCAACTCCGCCTCCTG |
| Region | | | GATTCAAGTGATTCTCCTGCCTCAACCTCCCCAGTAGCTGGGATTAC |
| | | | AGGTGATCCACCAGACCTGGCTAATTTTTTTTTTTTTTTTTTTTGTA |
| | | | TTTTAGTAGAGATGGGTTTTCACCATGTTGGCCAGACTGACCTCAGG |
| | | | CAATTTGCCCACYTCGGTCTCCCAAAGTGATGGGATTACAAGCATGA |
| | | | GCCACCGCACCAGGCCTATAAGTATTTTTGTAAGTAAAAACTATGTA |
| | | | TTTGAATATGTCTCAGGATTTTCAAGAAATGCAAGTAAAAAATAGGA |
| | | | GCTGTGAAATAATTTTTGATTGTTGGATTTTGTTTCTTTAACCACAA |
| | | | AATCACACATCAGTTGGACCATAAG |
| LNPEP | rs3096168 | 177 | GGAGTCCAGTGGTGTGATCTCAGCTCATTGCAACTCCGCCTCCTGGA |
| Region | | | TTCAAGTGATTCTCCTGCCTCAACCTCCCGAGTAGCTGGGATTACAG |
| | | | GTGATCCACCAGACCTGGCTAATTTTTTTTTTTTTTTTTTTTGTATT |
| | | | TTAGTAGAGATGGGTTTTCACCATGTTGGCCAGACTGACCTCAGGCA |
| | | | ATTTGCCCACCTYGGTCTCCCAAAGTGATGGGATTACAAGCATGAGC |
| | | | CACCGCACCAGGCCTATAAGTATTTTTGTAAGTAAAAACTATGTATT |
| | | | TGAATATGTCTCAGGATTTTCAAGAAATGCAAGTAAAAAATAGGAGC |
| | | | TGTGAAATAATTTTTGATTGTTGGATTTTGTTTCTTTAACCACAAAA |
| | | | TCACACATCAGTTGGACCATAAGTG |
| LNPEP | rs31398 | 178 | CCACTGCATTCCAGCCTGGGCAACTGAGCAAGACTCCATCTCAAAAA |
| Region | | | CAAAAAAAAGAATACCTAAAAACATTTTTTATATCAGAATTTTTATT |
| | | | CTTTCTAGTGGTATTCATAAAAGCATATTGCATATGATGCTTTTTAA |
| | | | AATATCATGTGCCCTCACCCCCCACCCGCCATGCACAACTTGCAGAA |
| | | | TGGAAATACTTCRACATGGTATTAACAGGTTTGGTGTTTTTATTTTG |
| | | | GAGAGAGATGAAAAAGGCGTCTGTTAGTACCTTAATACCGCAAGTAT |
| | | | ACGTTTAGCAATGACAGCCAATACCAATGGACTAGATTGGATGATAT |
| | | | TAATGAATAACTATTAGTTTTCTTCAGTGTGATAAGGTATTATGGTT |
| | | | CTGTAAGAGAATGTTCTGATATCTG |
| LNPEP | rs3214461 | 179 | TACTCATTAATTCTTTTTCAAATCCTTTAAAATAATTTTAAGACAGT |
| Region | | | TGAACACAGTCCACATCTATATGAGACTAAGTAGCAGTATATTATAA |
| | | | CTAAGTTCTACATATGAAAGTAAATTTTTAGAATGACTGTAGTTTGA |
| | | | ATTTTAGATTCCCAATTCGATAATCTATAGTATTCTATTATTTTCTT |
| | | | TCTTTTTCCTCAC/- |
| | | | TTTTTTTTTTAATAGGGATCTTCTCTCTTGTTGATGTTGAAAACTTA |
| | | | CCTTAGTGAAGATGTGTTTCAACATGCTGTTGTCCTTTACCTGCATA |
| | | | ATCACAGCTATGCATCTATTCAAAGTGATGATCTGTGGGATAGTTTT |
| | | | AATGAGGTAAGTGACCTGGGTAATTTATTTAGCTCTTACTGTAAAAA |
| | | | GAGAGGAGTTCG |
| LNPEP | rs33912722 | 180 | TTGGCCAGCAATTACCAGATCATTTTAGGCCAGCAGTGTAAATTCCT |
| Region | | | GTGTTATTTTTTGTCACATCATGCTTATAATCATCTCAAAAGATAAA |
| | | | GTAATCATCATTACTCTGTGTTTATAAGTGAGAAAACTGATACTAAG |
| | | | GGACAGATTTGCCCAAAGTCACCAAGTCAGTGAGAAAATCAGTACTT |
| | | | AAAATTTGTCTTT/- |
| | | | CTGACTAAGTCCAATAGTTATTCAATTATATCACAGCTAGTTCCTAG |
| | | | TTTTAAGAAAAGTCCCCCATCAATCTTCCCCTAAAGGTCCTAGATTT |
| | | | TGACCAACTCTCTTCTGACACCAAAGGGCCCTGTAGTATTAAAATAA |
| | | | TAAATTACTGAAAATATCTTGCCCACCATTGTGTCACATAAAGTCAA |
| | | | TTCTAATACATGTCAAT |
| LNPEP | rs33918743 | 181 | CAAAAACCAAATTTTAAAAATTAGTAGTTTTATCACCTAGGCAGAAA |
| Region | | | ACTTTTCTATTAGAAATTATACAGTCTCTCATCTCAAATACCATGTT |
| | | | TTACTGTTCTAAGAATCAAATAGTGCTATAGTGAAAAAAAGAGAGTA |
| | | | GTTTTTTTCTGAAACTATCTACTATACTGTATAACATGAGAAATTTC |
| | | | TCAAAAAATATGT/- |
| | | | TTTTTTTTTCCATAATCATGTCTGGTCTCTTTTTTGAGACCAAGATG |
| | | | AAACCATGTTGCTTGGTCTTCAACCATCAGCTTATCTGTTCTCTAAT |
| | | | GATTTTTGTTGTTCTGGTTTGGTCTTAAAGTTTTGAGAATTCATTTT |
| | | | ATTATAAATGTGAAAGTTCATTTAAATATTGTGTTCTTTTTGTTCCT |
| | | | GTAAAGGAAAAA |
| LNPEP | rs33934033 | 182 | AGGTTGCAGTGAGCCAAGATTGTGCCATTGCACTCCAGCCTGGGCAA |
| Region | | | CAGGAGTGAAACTCCATCTCAAAAACAAAACAAAACAAAACAAAACA |
| | | | GAAAACCCAAATTGGTGCTTCAAGAATATGATGTTATTTCTCAAAGG |
| | | | TACAATCTAGCTGAAATCATATACAAGTAAGTAGGTGTGGACTTTTA |
| | | | CTGTTGAGCTAARGTTTATGTTTATATATGTTTTATTCTTTAAGCTA |
| | | | AACAAACATTCAGATAACATTCTATGCATTTTTTGAAGCATAGGGTT |
| | | | AGTAATGAGGACTTAGATTTTTTAATTAAACAACTCAGTAACTATAT |
| | | | AAAAAGAAAAGGAGTCCCTTATGAATAAATATTAAAATTAAAAGAAA |
| | | | TAGGCAACTATAAAAGTAAGTATTT |
| LNPEP | rs34037881 | 183 | GGCAAAAAGAAACATTCCACTTGAATCTAACACTCTTTACAAAGATT |
| Region | | | TCCCACCCAATGACTTCAGCTAGACCAGAATGAGTCATAGCCTCACC |
| | | | AAGTCACAAGGTAGCCTGTAAGAAGTAACTCTCTTATCTGGACTTGT |
| | | | TGCCTTCCTGAATAAAATCAGGATTCCACTGGAACCAAGGAAGGGAA |
| | | | ATGGGTATCAGGA/- |
| | | | AGTGACTAGCTGTGTCTACTACATCCTGCTCTTCCCTTCCCCACTTG |
| | | | GGTGCTCACTGCACAGCCTGCAGCCATCCACCTAGGACAACTCTTCC |
| | | | CCAGGCTCCTCTCTTTCCACATTCCCTTGGTGACACTTCCCCTCATT |
| | | | GCAGCCACAATCCTCAGGGGCTTGTTTTCAGGCTCAGCACAGTATTG |
| | | | GATAGGAAAAGT |
| LNPEP | rs34323164 | 184 | TTGATGGGATTGTTTTATTTTCTTGCTGATTTGTTTGAGTTCCTTTT |
| Region | | | AGATTCTAGATATTAGCCTTTGTCAGATGTATAGATTATGAAGATTT |
| | | | TCTCCCACTTTGTGGGTTGTCTGTTCACTCTGCTGATTGTTGAATAA |
| | | | GATGTCCTTTCCCCACTTTATGTTTTTGCTTTGAGAAATTGTTGACA |
| | | | GTTTTAAAATCAT/- |
| | | | TAATGAGAAACTAAAATTGGAGTTAAGAGTTCACCAATGTGCTTTTT |
| | | | CCAAATTATGAATTGTTCAAAAAGTTTCCATTTTCCACCTGTTGAGA |
| | | | TCTTCATTTTGAGGTTTTTATTTTCTACTGTGTCTAATCTACATCCC |
| | | | ACTTTTCCAGGTGAGTATAGAGGGCTTTTTAAAATCAATTAGAAAAA |
| | | | AATAAATACTGTT |
| LNPEP | rs34701361 | 185 | AACTAAACATTTTTCTCCCTGTTTAGGATGACTATTTTTTGAATGTG |
| Region | | | TGTTTTGAAGTAATTACAAAAGATTCATTGAATTCATCCCGCCCTAT |
| | | | CTCCAAACCAGCGGAAACCCCGACTCAAATACAGGAAATGTTTGATG |
| | | | AAGTTTCCTATAACAAGGTAGTAAATATCAGGTGCAGGTGGAAGCTC |
| | | | TGCTTTCAGAAGA/- |
| | | | AAGTAATAATGACTATAGAATCAGCAGTTTAAGTCACTGGTGCCAGT |
| | | | TCCTGCTACTTGTTTCACTTTTTATTTGTTCACTTTTCAGACATCAC |
| | | | ACATGTTCCGTAAAATTTAAGCTTCCTTTAAAAACATACCATACTAC |
| | | | CATTTTCTTTATCTCTTTTTTCAACTCTTTTGTTTTTTTTTAAAGGG |
| | | | AGCTTGTATTTTGA |
| LNPEP | rs34815125 | 186 | CTCTTATCAGAACGTAAAATGTGCCAGACTCTTAGTTAAATCTCTCC |
| Region | | | TGGATCAAAAAAAGACCTGGGGTGGTGCAGTGGCTCACACCTGTAAT |
| | | | CCTAGCACTTTGGGAGGCCAAGGCAGGAAGATTGCTTGAGGCCAGCA |
| | | | GTTCAAGACCAGCCTGGGCAACATAGTGAGAGCCTGTCTCTACAAAA |
| | | | AAATTAAAAATTA/- |
| | | | AAAAAAAAAATTAGTCAGGTGTGATGGTATGCACCTGTGGTCCCAGC |
| | | | TGCTTGAGAGGCTGAGGTGAAAGGATCACTTGAGCCTGGGCAAAGTG |
| | | | GAAGTGAGCTGTGGTCATGCCACTGCACTGCAGCCTGGGCAAGAGAG |
| | | | TGAGACCCTATCTCAAAAAAAAAAAAAAAAAAAGAGATCAGAAAGGT |
| | | | CTTTTTCTATAG |
| LNPEP | rs34962665 | 187 | CAGATGCCTTGGTTATGTGCGGATTCTACCGTCATTTATTTCAGCCC |
| Region | | | TAGATGGTGCTAAAGTAGAGACAGACAGATTTTTCTTAAACTATTGC |
| | | | CTTTAAAAATCATTTATTTTTATCCCCATTTTTTTTGTTTATATCCA |
| | | | AAGGGTTTTCAACAAGCTGCCCCTTTCCCAACACCCCAGCCCCTCAA |
| | | | CGAAACATAATAG/- |
| | | | GAGACACATCATTTAATTTCTCAGCCCTTTCATGATCTCTTAGACTA |
| | | | ATCTTAGTTTTCATAAATTAAAGGCCTACTTGGCTAAGTTCATTTAC |
| | | | TTTTTTTTTCTCCTACTTTTCTTGATCTCTGGACCCAGGAATCCCAG |
| | | | ATGATACAAAACCCTTTGTTTCATACCTGCCCTGCCATAGAATGATC |
| | | | TAGACCTTTAAG |
| LNPEP | rs35199417 | 188 | GAAGTAAGTAGAGGCAGGTGGTAGGGTGGCAGTAAGAATTGATTCCC |
| Region | | | CCAAATTAACTATGCTGTTTGTCCTAATTTTATATGTGTTGTAGCTT |
| | | | TACCCTTCAAAAAGAAAGAAACTTAGTTCTATTTACAAAGGTAGTAA |
| | | | ATTCAGTTTGATTTAATTGTGCTTTCAAAAGTAGTGTAAAGGGAAAA |
| | | | GAACCGAACCTTA/- |
| | | | AAAAAATTCTGTAAGAATATTATAAACTCAAAATTTATTTCCATGGC |
| | | | TTTTGACATATTGAAAATAAACTGGGGATAAATACCTACCTTGACCA |
| | | | GCAACCTTTACACCAGTAGCCATAAAATGAGGCCATTCAGATAATGT |
| | | | TATTGAAAGAGGTGAAGTTCAATGCCATTCGTAGTAATAATAATATC |
| | | | TGGTATCCAAAG |
| LNPEP | rs35304156 | 189 | GTAGGTTACTGATTTGCCCAAAGTCATGTCGTTAGTAAATTATAGAG |
| Region | | | TCTGGGTCTTCTGACTCCAAATCTCATACTCTTTCTTTTCTCCTTAT |
| | | | CTTCTAGTAGTGGAAACTAAGCCCAAAATGAGAGAGGCTACCACCTC |
| | | | CAAGTGGTGGTTGTATATGTGCTATATTGATTGGTACCTGAAATATG |
| | | | CACACCAGGGCCAT/- |
| | | | TATATTTGCCGTGATTATAGCCACGCTGGGATGATCTCCCAAGTTCA |
| | | | GATCTAGTTATTCTTTTACTTAACTGAAAATCTGCATTTCTCCTTGT |
| | | | TTCTTTTTATGCTTTTCCACCAACCTGTAATCGAGGACTTTTCTTTT |
| | | | TTTTTCCCTTGAGACAGCATCTTGCTCTGTCGCCCAGGTTGGAGTGC |
| | | | AGTGGTGCAATC |
| LNPEP | rs35475916 | 190 | ACTACCTCATAGAAGAAAATATTTAAAGCTCTTTCTGACTTCATTTG |
| Region | | | TTTATATATGCCATCTTTTTTTTTTTGTTTTTAAAGAAACAAGATCT |
| | | | CACTCTGTCACCCAGGCTGGAATGCAGTGGCATGATCATAGCTCACT |
| | | | GCAATTTTGAACTCTTAGGCTCAACTGATCCTCCCGCCTCATCCTCC |
| | | | CGAGTAGCTAGGMCAACAGGCATACATCACCATGCCTGGCTTAATTT |
| | | | TTTTGTAGAGACAGAGTCTCTCTATGTTGCCCATGCTGGCTTGAACT |
| | | | CCTGGCCTTAAGCAATCCTCCTGCCTTGCCCTCCTAAAGCACTGGGA |
| | | | TTACAGGTGTAAGCCACGATGCCCAGCCTGTATATGTCAACTTAGTC |
| | | | TTAAGGAATGTTGTTTGAATTCTGT |
| LNPEP | rs35562078 | 191 | GTCTCACTATAAAAATTTCTAGGAAAAGAACATGCAAAGCCTGATAA |
| Region | | | AATGATGTTTTCTTTTTCTCTTCCTCTTCTTAGTAAAGAGGAATATA |
| | | | CAAAATTCACTAGAATATAATTGATTTAATCTAGAGCTGGAACTGGG |
| | | | CCAATACATGATGAAAGTAGTGTCTGTTACTTCCTCTTCTCAACTGT |
| | | | GTTATTTCCCTTGCT/- |
| | | | CTGCTGCTGCTGCTATTTTAATTCCTGCCATTTCGGGTTTAGAGAGT |
| | | | CCACATGAAAACTTCTGTCCTTACGTTTGACCCTGAGGACAGCTGAG |
| | | | CCTTCTTGGTTCCTAATGCTCCAGTGAGAATTACTCTTAATTTAACT |
| | | | GCATTTTTATTTTTTCTATTCTCAAAAGAAAGGTAGCAGAGAGGGTG |
| | | | ACTTCAGGCTTC |
| LNPEP | rs35929998 | 192 | AAATGAGGTTTCACCATGTTGGCCAGGTGAACTCCTGACCTCAAGTG |
| Region | | | ATCCGCTCACCTTAGCCTCCCAAAGTACTGGGATTACAGGCATGAGC |
| | | | CACCGCGCCCAGCTGAAAGTATATATACTTTCTAGTTTGTGATATAT |
| | | | TCTAAAGTATATCTAAAGGTTGGTATTTTGGCATTTTGAGGTCCCAG |
| | | | AACTGAAAACATT/- |
| | | | AATTAATAGTTTTTTTTTTCATATGAATGTAGGTCCTATGAATCACT |
| | | | TTTATTACCGCAGTGTGGTGCATAATCAAACAAGGAGGACTTAGTTG |
| | | | TCTTAAAAAATTATTTTTGCTTTCATGTTCAGATTGGTATCCAGTTG |
| | | | AAAGTATTTTTCAACTTCAAAAATGGGAAGTCTGGGCAACAAATGAG |
| | | | ATATTTGTGGGTTGTA |
| LNPEP | rs36019589 | 193 | AACCCTTTGTTTCATACCTGCCCTGCCATAGAATGATCTAGACCTTT |
| Region | | | AAGAGGACTAGAATCAGCCCTCTTTTTCTGGGCTTTCTGGGGCCAGG |
| | | | AATGACTAGGATTGATCTGCTTTCTCAAGCTTTGCCCCGGGCCTAAC |
| | | | CAGGTCAGCCTGGGACCAGCCCGTGGGGTTTGACTATACCTGGAACA |
| | | | GATGGTTAATCTA/- |
| | | | TTGGCTTGCTATAATGTAATTTCCATTTGGCTGGCAGTAGGGAAAGG |
| | | | AAGGTACTTCCTGTAAGCTACACACTGATTTTCATCCAGGTGTTCAC |
| | | | ACATACCGGGTTTTATGAAAGAGAGCTTGACCCTCGCATTCCTGATT |
| | | | AGCATTTTGTTAGTGTGAAAGTAAGGTATAGACACAGAGACAGGTAT |
| | | | AATCACAAAATG |
| LNPEP | rs3734015 | 194 | CCCAGGATCATCAACCCATCAAATTTCACTTGAAGTATTTCATTATG |
| Region | | | GCCATGTAAGCACAGGTTCCAACTGAAGGAAGAGTGATTTTGCCCTA |
| | | | GATTGGAATGCCAGAGTACCAGGGGATATAAGGAGAAATATTTTTAG |
| | | | TAGAAATCTTTATTTGTAAGGTTTCCAATTCTGTGCTTCATGTGTCT |
| | | | GTATAGTCACTTYCCTTCTTTTCCCAAATGACATTTGAAGGCTTTGC |
| | | | TTTGAAAGGTTTTAGAGGATAAATTTAATGGCTACTTCTCGTAATAA |
| | | | AATTCCAGTATGCACACCACAGTTCAGAGACTGAGTACTGTGCTACT |
| | | | TGACGTTGTGTTAGGTTTAGTAGTCTCTAAGTTCCCCTCTAGAGGTA |
| | | | AATGAGATGATTTATTTTGTTTCAG |
| LNPEP | rs3797796 | 195 | TCAAAGCCATTTTTTTGTCTTTCTCTCTTTTAATTTTGCTTAGTTCT |
| Region | | | ATTAGAGAAGCTTTTATAAATTTTTCTTCTCTGAGGTATGATTAGAA |
| | | | TACATATTTATACTGGTAGATAAAGTAATTAAGGGATGTATTTCTTG |
| | | | TTTTTACACATAGCTTACATTTCCTGGGGATAATAGGCATTATAGAA |
| | | | GGAGAACTAAAGRCAAGAACTTTCAAGTTCCCATTGCAATTATACAT |
| | | | TTGTGTTCAATCCCAGATCTCACGCAAGAATTGAAATGCAGGGCCAG |
| | | | TATGCCATTTATTTTAAAAGTATTACATAGAGGGAAAATAAAATAAA |
| | | | AATTATTTATCTGAATAGAATTATGGATCTTGCTTGGTCTCTTTCTC |
| | | | CATTTAAGAAGGATCAAAAAGTTTC |
| LNPEP | rs38029 | 196 | CGTCTGCCTTTTTCTTGTTGATTTGTAAGAGTTCTGTATATATCCTA |
| Region | | | GATATGAATCTTTTGTTGGTCATGTATATTTGCAAATATCTTCTCCC |
| | | | ACTCCATCTTGCTTTTTTTTACTCTCTTAATGATTTTTTATTAATAT |
| | | | GAGTTTTAAATTTTAATGTAATCTAGCTTATGAAATCTTTTCCTACC |
| | | | CCTAGATATTCTSTGTTCTCTTCTGAAAACTTTATCATTTTATCCTT |
| | | | TACATTTAGATCTGTGATCCATCTGGAATTGATTTTTGTGGATGGTG |
| | | | TGAGGTAGACACCAAGATTCATTCTTTTCAGTATGGATATCCAGTTA |
| | | | TCCCCAGGACCAGTATATTTTATTGCATAGAATTATGTTTGAGGTAA |
| | | | TTAGTATGATATCAACACCATTTGG |
| LNPEP | rs38030 | 197 | ATAACAGAAATTTATTCTCTCATAGTTCTGGAAGCCAGAAGGCCAAA |
| Region | | | ATCAAGGTATTGGCAGAGTAAGGTTTGCTCCTTCTGAGGAAGAATCT |
| | | | GTTCCATTCCCCTCTCCTAACTTCAAGTGATTGCCAGTAATCCTTGG |
| | | | TATTCCTGGGCATGTAGGTGACTAACCGTGGCCTTTGTCTCTGTCAA |
| | | | CACAGTGTTCTCY/- |
| | | | CTGTGTTTCTGTGCCCAAATTGCCCCATTCTTAGATTAAGGCCCACC |
| | | | ATAATCCAGTATGACCTCATCTTAACTTGATTGTACCTGCAAAGACC |
| | | | CTATTCCTAAATGAGGTCATATTCATAGGTCCCAGGCAGACACAAAA |
| | | | TTTGAGGGGATACTATTCAACCTAGTACAGGTAGCAATAAATAAGAT |
| | | | TAGTGCATATCA |
| LNPEP | rs38031 | 198 | TCATATGGAGACTAACTAGTAAAATTGCTCCCTGTAATTCGGTGGTG |
| Region | | | TAACTGCTCAGGAATTAGCCACAGCCATCTTCAAGTGTCAGATTTCC |
| | | | TTTGCTTCCAGGACTTCAAGTGCCATTCTTTCCATTGCTGCCTTTGT |
| | | | GTTTTAGTAAACTCTCAATGAGTATTGCCATTGTTCTTCACTGATTT |
| | | | TTTTTTTAAATARGATTTCAAGCATGTGATTTTTTTTCTCACATTCT |
| | | | TCATTTGTTCCTATTTGACAGTTATGAGTAGGATTTGAATTTCTTTT |
| | | | GTTCTCCAGTCATTTGGAATGGTTTTCTATCATAATGCTATTGAGAA |
| | | | GGTAAGGCCAGTGAAGACACCACATAACAATGCAGTTAGGTATGTCA |
| | | | AGTGGGATCCCCTGCATTGCTTGTC |
| LNPEP | rs38032 | 199 | CCTACTTTAATTTGTGGTTGGAAAATTCTATAAGGTTGCCTACATTC |
| Region | | | CCTCATTTTGTGTCTGCTGCAGACTTCTCTAATGACTTACTACTGAC |
| | | | TTTGTTCCCACTAAGCTTTCTTGGGGGTCCTCAACATGGCACCCCAT |
| | | | GAAGCCATTTCAGATCATTTGAAGGGATGTCGCAGCTAGAGCTCCTT |
| | | | CTGTGGATGTATYTGTAGCAGTAGAGTGGAGCAATCCCAGGTCATAA |
| | | | GGAAGGATTTTGGTTTTGGAGGTGTTCTAATGGGAGAAGCAGAACCA |
| | | | ATGTGACTATCTTTAACTTAACATTTATTTGGTCATCTTTGGGACTA |
| | | | AAAACTCCTTGAGGAGTTTCACTGTGCTCCATATGTCCTCAGGATGA |
| | | | AGGATGGTACAACAGACTGAGACTA |
| LNPEP | rs38033 | 200 | TTTGGAGGTGTTCTAATGGGAGAAGCAGAACCAATGTGACTATCTTT |
| Region | | | AACTTAACATTTATTTGGTCATCTTTGGGACTAAAAACTCCTTGAGG |
| | | | AGTTTCACTGTGCTCCATATGTCCTCAGGATGAAGGATGGTACAAACA |
| | | | GACTGAGACTAGGAGCCATGCTCTTTGCAGAAATTCATACTGAGAGG |
| | | | TTATAATATGCTRGCATCTTTACCATTTATTTCCTATTTGAATTTTC |
| | | | AGTTTCTCAGTTTGTTTGTTATTGCCATTTATTCCTATAGTTACAGA |
| | | | ACTGTCTTTTCCCCTTTGCTTGTAGAAGTCATCAGTCGTTCTAGATG |
| | | | ATGGACTTGTTCAGGATGAGTTTTCTGAGAGTGTGAAGATGAGCACT |
| | | | TACTTGGTTGCTTTCATTGTGGGAG |
| LNPEP | rs38034 | 201 | CTGTCTTTTCCCCTTTGCTTGTAGAAGTCATCAGTCGTTCTAGATGA |
| Region | | | TGGACTTGTTCAGGATGAGTTTTCTGAGAGTGTGAAGATGAGCACTT |
| | | | ACTTGGTTGCTTTCATTGTGGGAGAGATGAAGAACCTGAGTCAGGAC |
| | | | GTAAATGGAACCCTGGTATGTTGATGTGGTAATTGTCTGAAAGCCTG |
| | | | TGTCACAAGAGGYTCAGAGGACCTCTTGCTTTAACGATTCCTGGTAT |
| | | | TTGCTGTGTGAAATAAATAAGCTTTTAGATCACACTCTGACATTTTA |
| | | | TACCAGAAATGCTACTTTTTTGCTTAGCTGTTATTTACTGTTACTAG |
| | | | TTTAATAGCTGAAAGTCAATAATTTTCAAGTTTTAAAAAATTTTACT |
| | | | TTTAAAGAGAATTTTAGTAAGACAC |
| LNPEP | rs38035 | 202 | AAACAATTATTCTTGATGATAGAAATATGATAGAATGTCTTAGTTTC |
| Region | | | TGTTTTCGTATTTTTGAGCTCTACCAGGGAATATACTGCTAGTTTTG |
| | | | GGTTTCCTTTCTAGACTAAAGAGCTTTATATTAATCACAGGATACTT |
| | | | GGATCTTATCATTTTGCTACTTCAAAAGGGTATATGTTTCCTATTAG |
| | | | AAAAGACTATCAYGTCTATCCCATACCTTTCAGAGATAAGGACAGAG |
| | | | ATAAGGATTCTCTGGTGATTTATCAGTAATAATACTGTATGCCTTTA |
| | | | ATGATGCTACTCAAAAAGTAAACTAAGTTTTTAATGGTAAGTGTAGA |
| | | | CTGTAATATTAAGCGCTAAATAATGGTTCACTCACCTTTGGATTGAA |
| | | | AGACTTTATGTCAAGGATTTTTCAG |
| LNPEP | rs38036 | 203 | ATAAGGACAGAGATAAGGATTCTCTGGTGATTTATCAGTAATAATAC |
| Region | | | TGTATGCCTTTAATGATGCTACTCAAAAAGTAAACTAAGTTTTTAAT |
| | | | GGTAAGTGTAGACTGTAATATTAAGCGCTAAATAATGGTTCACTCAC |
| | | | CTTTGGATTGAAAGACTTTATGTCAAGGATTTTTCAGAATCCTTTCA |
| | | | AAAGGATATTATRACTGGCTTAAATCTGAAATAATTCAATTAATTCC |
| | | | ACTTCAGGTGTTGCACTACATATTTAGCCTTTGATTTAGAGTTTGCA |
| | | | GCCTTGATAAAGCCTAAGAAGCCCAATCTAAAAGAGTCAGGTTTGCT |
| | | | GCTGCTTCAAGACTCAGCTGAATACTACGTTCTCCATGAAGCATTTC |
| | | | TTTCTTTCCCCAGCTGGAATTAATC |
| LNPEP | rs38040 | 204 | TCTGTTCACTCTGCTGATTGTTGAATAAGATGTCCTTTCCCCACTTT |
| Region | | | ATGTTTTTGCTTTGAGAAATTGTTGACAGTTTTAAAATCATAATGAG |
| | | | AAACTAAAATTGGAGTTAAGAGTTCACCAATGTGCTTTTTCCAAATT |
| | | | ATGAATTGTTCAAAAAGTTTCCATTTTCCACCTGTTGAGATCTTCAT |
| | | | TTTGAGGTTTTTRTTTTCTACTGTGTCTAATCTACATCCCACTTTTC |
| | | | CAGGTGAGTATAGAGGGCTTTTTAAAATCAATTAGAAAAAAATAAAT |
| | | | ACTGTTTTGTAAAACCCATTGCTTTGAACATGGCTGTTTAACACTTG |
| | | | CCTTTTGATACTTCCTGAATAAAATGTTTATAGTTTGTCGCATCATA |
| | | | TATGTTTAATTTATTCATTTAGCCA |
| LNPEP | rs38042 | 205 | TTATCTAGTATCCCACTTCTCTTAATTACACACGAATATTTTTGCCAA |
| Region | | | ATTCCCAATTCTGAAACAAGTAGTTACCATGTTGACAGGGGTTGACA |
| | | | ATGATATGGAAACTACATATTCAGAAGACACTGAATTCTGGGTTTAG |
| | | | AGGGTTTGGGTCAGTGGCAGACAGAACTCTGTTATGACTCTGTTCTG |
| | | | TTATTATATAATRAACTTAGAGCATTTTAAGCAGGTTTTCAAATCCT |
| | | | GAAATAACTGCTCTAAGTTTGGTATAATAACAGAACCTCAAGTTTTA |
| | | | AATTTTCTTTATTGACAGGTCCACTATGGTCTTCAAAAATCACACCA |
| | | | GTAGCTCTAATTGGAATAGATTTAATATAGCTAACTGAACTCCTGAT |
| | | | TTTCTTGTTTGTTAATAGTACCTGT |
| LNPEP | rs38043 | 206 | ATACGAGATTGAGATAAAAGGTTGCTTTGCATGTTCAAGCACCAGCA |
| Region | | | AGGATCCAGTGTTATCTGAAGTAGAGTAAGCAGAGGAGAAAGTAGTA |
| | | | GATGAAGTTAGAGGGACTGTAAGAGGCCAGATTATTATAGGGTCTGC |
| | | | TAAGCCATAGTAAGGACCTTGATTTTATTCTAAGTGAAACGAAAAGC |
| | | | AATTTGATCAGGRAAGTACCGTGATATGGCTTATTTTGTAAAAGATC |
| | | | ACTCTAGTCTTCAACAGTACATGGAGTAAAGAGGACTAGTTAGGAAG |
| | | | TTATTGTAATAGATGGGTGGCGAGATAATGGTAGCCTGGACAAGGGT |
| | | | GGAAGTTGTGAAGGTGATGGAGGTACAACTGGACTTGGAGTGTGTTT |
| | | | TAAAGATTGATCCAGTAGAATTTGT |
| LNPEP | rs38044 | 207 | TCCCAGCACTTTGGGAGGCCAAGGTGGGTGGATCACCTGAGGTCAGC |
| Region | | | AGTTCAAGAACAGTCTGTCCAACATGGTGAAACCCCGTCTCTACTAA |
| | | | AAATACAAAAATTAGCTGGGCGTGGTGGTGGTGCCTGTAATCCCAGC |
| | | | TACTCAGGAGGCTGAAGCAGGAGAATCGCTTGAACCCAGGAGGCGGA |
| | | | GGTTGTAGTGAGYGGAGGTCGCACCACTGCACTCCAGCCTGGGTGAC |
| | | | AAGAGTGAGACTTCATCTCAAAATAAATAAATAAATAAATAAATACT |
| | | | TACAGTAGAGTGATGATTAGAAGATGGCTCAAGAGAAAATGGAAAGA |
| | | | GAGCAAATGGAGATGGCAAATTGAGGACAACTCTTTTGAGGAGTTTT |
| | | | ACTACAATGGGGGAACAAAAAAACA |
| LNPEP | rs3849749 | 208 | ACTCGGGAGGTTGAGGCAGGAGAATCACTTGAATCCAGGAGGCGGAG |
| Region | | | TTGCAATGAGCTGAGATCACACCACTGGACTCCAGCCTGGTGACAGA |
| | | | GTGAGACTCTGTCTTAAAACAAAACAAAACAAACAAACAAACAAAAA |
| | | | ACATATAAAGATGCTCTTTACTATCCATTTCCATCACCCACCGTCAG |
| | | | TGGTCCAGACACWCTTTCTCCATGCTTCCGCTTAAGCTTCTCAGCAC |
| | | | CAAGTATTGTGTTGCTTCTGTCTCTCATCCCTCTCCATTTCCCTCTC |
| | | | CCTTGCCATGTGTGTGTGCATGTATGTATATTTGTAGACATCAGTTT |
| | | | AGCTCCCCTCCAACACGGAAGAATCTATCATTTGGTGTGCATACTGG |
| | | | CAGTAGAGGGTGGGAGTTAAAAAGA |
| LNPEP | rs3849750 | 209 | AGTTGCAATGAGCTGACATCACACCACTGGACTCCAGCCTGGTGACA |
| Region | | | GAGTGAGACTCTGTCTTAAAACAAAACAAAACAAACAAACAAACAAA |
| | | | AAACATATAAAGATGCTCTTTACTATCCATTTCCATCACCCACCGTC |
| | | | AGTGGTCCAGACACACTTTCTCCATGCTTCCGCTTAAGCTTCTCAGC |
| | | | ACCAAGTATTGTRTTGCTTCTGTCTCTCATCCCTCTCCATTTCCCTC |
| | | | TCCCTTGCCATGTGTGTGTGCATGTATGTATATTTGTAGACATCAGT |
| | | | TTAGCTCCCCTCCAACACGGAAGAATCTATCATTTGGTGTGCATACT |
| | | | GGCAGTAGAGGGTGGGAGTTAAAAAGAAAATTTGGCCAGCAATTACC |
| | | | AGATCATTTTAGGCCAGCAGTGTAA |
| LNPEP | rs3909451 | 210 | GTTGACCATACCAGTTAATCTTATTTACAGAGGATGTGGAGATAAT |
| Region | | | GATTAATATGTTGAGCTGATGAAGTAGACAAGTGGCTGCTGTATGTA |
| | | | GAAGTAATGTTGGAACAAATAATACGTCCCAGAATAGTTCTGTAAG |
| | | | GCTGATTTTACTCTGAAATTTTAATTAATTTATAGTTAATATAACTA |
| | | | CCTCTGTATTTTKTTGTAGTCTTTTGTGGGTAGAGTTGAGGAAGAGA |
| | | | TAGGAATGGGATTATTTTGACATGGCTCATGATCACCAAAATGTGAT |
| | | | CCTTTGGTCAGTTTACCTAAATATCAATGTAATTATGTTTATCTAATT |
| | | | TAATAATTTGCTGAAATCTTCCTTATTTTTTACTTTTTATGAAGCTT |
| | | | TTAGCCATTTATATTAGATGGTGAT |
| LNPEP | rs39602 | 211 | TCATTTTGTTGCCCATTCAGAGAGCTTGTAAGCTTGGGCTCTGCCGC |
| Region | | | TTTTGCAAAAGCCAAGGTAAAGCCAGGATCGCTGCCAAGTTGTTTGC |
| | | | ACTCTTTGGAGTTCTAGTTAGCTCAGGGCCTGACTGTATTTTTCATC |
| | | | CATCTTTTCTGAAGTGTCTTTGGGCAGTATGTAGTTATTTATTACAA |
| | | | AATTATATTCACSTAAATGCCAACCATCTACAAAAACAATGAGTAAT |
| | | | TTTTCTACTTTGAAGATACACAGATGGGGACAAAAACCCTGTTTTGG |
| | | | AATTCTGTTCTATTCCTCAGTATCCAGAAAGTTACTGACACAGTAAA |
| | | | ACAAGGAAAGTTCTACCCTAAGAGCCGCCATCACTTCAGGCCGCTGG |
| | | | TTTGTCAGCCATCTGTTGCTTCTTA |
| LNPEP | rs3985004 | 212 | TTGACATGTATTAGAATTGACTTTATGTGACACAATGGTGGGCAAGA |
| Region | | | TATTTTCAGTAATTTATTATTTTAATACTACAGGGCCCTTTGGTGTC |
| | | | AGAAGAGAGTTGGTCAAAATCTAGGACCTTTAGGGGAAGATTGATGG |
| | | | GGGACTTTTCTTAAAACTAGGAACTAGCTGTGATATAATTGAATAAC |
| | | | TATTGGACTTAGG/- |
| | | | TCAGAAGACAAATTTTAAGTACTGATTTTCTCACTGACTTGGTGACT |
| | | | TTGGGCAAATCTGTCCCTTAGTATCAGTTTTCTCACTTATAAACACA |
| | | | GAGTAATGATGATTACTTTATCTTTTGAGATGATTATAAGCATGATG |
| | | | TGACAAAAAATAACACAGGAATTTACACTGCTGGCCTAAAATGATCT |
| | | | GGTAATTGCTGGCCAAA |
| LNPEP | rs42983 | 213 | GAGCAGGTGATCAGTTATATCAAATGCTATCAATAGGTTGATAAGAT |
| Region | | | GAGCCTGAGAATTCACATTTGTATGGCACCAGGAAGTTTACCAGTGA |
| | | | CCTTGATAAAAATACTTCCGGCCGGGCATGGTAGCTCACGCCTGTAA |
| | | | TCCCAGCACTTTGGGAGGCCAAGGTGGGTGGATCACCTGAGGTCAGC |
| | | | AGTTCAAGAACASTCTGTCCAACATGGTGAAACCCCGTCTCTACTAA |
| | | | AAATACAAAAATTAGCTGGGCGTGGTGGTGGTGCCTGTAATCCCAGC |
| | | | TACTCAGGAGGCTGAAGCAGGAGAATCGCTTGAACCCAGGAGGCGGA |
| | | | GGTTGTAGTGAGTGGAGGTCGCACCACTGCACTCCAGCCTGGGTGAC |
| | | | AAGAGTGAGACTTCATCTCAAAATA |
| LNPEP | rs430827 | 214 | TACCTCATAGAAGAAAATATTTAAAGCTCTTTCTGACTTCATTTGTT |
| Region | | | TATATATGCCATCTTTTTTTTTTTGTTTTTAAAGAAACAAGATCTCA |
| | | | CTCTGTCACCCAGGCTGGAATGCAGTGGCATGATCATAGCTCACTGC |
| | | | AATTTTGAACTCTTAGGCTCAACTGATCCTCCCGCCTCATCCTCCCG |
| | | | AGTAGCTAGGCCMACAGGCATACATCACCATGCCTGGCTTAATTTTT |
| | | | TTGTAGAGACAGAGTCTCTCTATGTTGCCCATGCTGGCTGAACTCC |
| | | | TGGCCTTAAGCAATCCTCCTGCCTTGCCCTCCTAAAGCACTCGGATT |
| | | | ACAGGTGTAAGCCACGATGCCCAGCCTGTATATGTCAACTTAGTCTT |
| | | | AAGGAATGTTGTTTGAATTCTGTTT |
| LNPEP | rs4360063 | 215 | TTGTTTTGGCTGGCGATCACCCTGCTCAGGTTCAGACCTTAGTTCTG |
| Region | | | TTTCACCATCTGTGGCCAGTGGCTCCAATGTTAGTTTAGTTCTCCTA |
| | | | GCCTTTGTATGGTAGGCAGAATAATGGTCTCCAAAGATGTCCATTTC |
| | | | CTAATCCCTGAAGCCTTGGTAATATTTTAGGTTACATAATGAAGAGG |
| | | | AGTTAGGTTGCARTTAGAGTTGCGGTTGCTAATCAGCTGACCTTAAA |
| | | | ATAAAGAGGTTATCCTGGATTATCTAGTTAGGCCCAGTGTAGTCATA |
| | | | AGGTTTTTAAAAGTGAGAAAGTGAGGCAGAAGAGTCAGTATCAGAGT |
| | | | GACAAAGTGTGAGAAAGATTCAGCCTGCACTTGTGGCTTTGATGATG |
| | | | GGAGGGGGGCCCAAGCTAAGGAATG |
| LNPEP | rs4869314 | 216 | CTCTTATTTAAAACATTTTAACTTTATCCTTTATCGTCACCACAATA |
| Region | | | ATGAGCTGTTGTTCTTTAAAGCAGTGAACTAAATACTCTGTTACACA |
| | | | GAGAGCCATGCTCAACACTGTGCTTCGAGAACACATGGGCTGCTTCC |
| | | | TTTGGTTCAAAATCTCCCCACTGGCGCATTTTAGGTGTTTTGATCAT |
| | | | GAGTCACCAGGAKCTCTAAAGCACTTAACTGAGTCTGGGGATTTCTA |
| | | | ATCTTTCTGCCAGTTGTTTGTAGGGAAGTGCTCTGTGAGCTCTACCT |
| | | | CTGAGGCTCCATGCTCCCTCTGGCCCTCCCTTTAATAGCTTCTCTTC |
| | | | CACGGAGATGCAGTCAAGTGCTGAAGCAGCAAACAGCACTGGAATTT |
| | | | TTGCCCCCACTTTTTTGTCTTCCCA |
| LNPEP | rs4869315 | 217 | ATGAGCTGTTGTTCTTTAAAGCAGTGAACTAAATACTCTGTTACACA |
| Region | | | GAGACCCATGCTCAACACTGTGCTTCGAGAACACATGGGCTGCTTCC |
| | | | TTTGGTTCAAAATCTCCCCACTGGCGCATTTTAGGTGTTTTGATCAT |
| | | | GAGTCACCAGGAGCTCTAAAGCACTTAACTGAGTCTGGGGATTTCTA |
| | | | ATCTTTCTGCCARTTGTTTGTAGGGAAGTGCTCTGTGAGCTCTACCT |
| | | | CTGAGGCTCCATGCTCCCTCTGGCCCTCCCTTTAATAGCTTCTCTTC |
| | | | CACGGAGATGCAGTCAAGTGCTGAAGCAGCAAACAGCACTGGAATTT |
| | | | TTGCCCCCACTTTTTTGTCTTCCCATTGATTACCATGTTAACATGTC |
| | | | ACTCTGTGCATAACCCTGGCAAAGA |
| LNPEP | rs4869316 | 218 | TAGCACCTAGCATATGTTGATCTTATAATAGTGATTAATAAGCAGTT |
| Region | | | AATGATTGATTAAAGAACTTATGGTCTGTCTTTGGGATTCATGTAGA |
| | | | TAATAGGAAAGGCAAAGCAGAAAAATTCAGTTAATTCAGATGATTCT |
| | | | AATAATTATTAAAATATTTTAAAATTTCCAACTGCAAAGAAAATAAT |
| | | | TTTTTATAGAACSATTAGACCCAGAGAACTCATACCTGTAATTAGAA |
| | | | GAACCCTAAGTCATTGTAGACAGAAGAGATCCTTTCTTTTTTACAAG |
| | | | CACTTGTGTCCCAGGGACAGTAATAATATTGTTTAATATTTCTGCAG |
| | | | CAGTTTACAGTTTAAAGACACTTTCATGGCCGGGTACAATGGCTCAC |
| | | | GCCTGTAATCCCAAGACTTTGGGAC |
| LNPEP | rs5869737 | 219 | TGCCACCCAACTTTTAAGATCCAGCTGAGATTTCACCTCCTCCTTAC |
| Region | | | AGACTGTTCCAGCCCTCATTCGTTTGCCTGTCTGCTAAATTCTTAGC |
| | | | AGTGCACTTATAATCTGTTCCACATAATAGTACCCTCTTTTATTGTT |
| | | | TTTATTAGTTCAATAATGATAATGTGCTTAAGAACAAAAATTGTGTC |
| | | | TATTCTTTTTTTT/- |
| | | | ATGTGATAGCACCTAGCATATGTTGATCTTATAATAGTGATTAATAA |
| | | | GCAGTTAATGATTGATTAAAGAACTTATGGTCTGTCTTTGGGCATTCA |
| | | | TGTAGATAATAGGAAAGGCAAAGCAGAAAAATTCAGTTAATTCAGAT |
| | | | GATTCTAATAATTATTAAAATATTTTAAAATTTCCAACTGCAAAGAA |
| | | | AATAATTTTTTA |
| LNPEP | rs5869740 | 220 | GAAATGCCTATACCTGTGTGTATGTAATTTGCAAGCTCTTTTGAAAA |
| Region | | | TTTTTGGAAGACGAAGTGGTTTTATTGTTTCTTTATTTTTGAAACTG |
| | | | CCTCGCTCTGTCAGCCAGGCTGGAGTGCAGTGGCACCATCTTGGCTC |
| | | | ATTGTAACCTCCACCTGCTGGGTTCAAGCAATCCTCCCGCCTCAGCC |
| | | | TTCCAAGTAGCTG/- |
| | | | GGACTACAGGCATGCACCATCATGTCCGACTAATTTTTGTTGTTGTT |
| | | | GTTGTTATTTTTTGTAGAGTCAGGGGTTCTGGCATGTTGCCTAGGCT |
| | | | CGTATTGAACTCCTGAGCTCAATTGATCTGCCCACCTTGGCCTCCCG |
| | | | AAGTGCTGGGATTACAGGTGTGAACCACCACACTCGGCCAAGACAAA |
| | | | GTGTTAGTAATT |
| LNPEP | rs6556942 | 221 | GATTCTCATAAGGAACACGCAACTTAGATCCCTCACATGCGCAGTTC |
| Region | | | ACAATAGGATTCATGCTCCTATGAGAATCTAATGACACCTCTGATCT |
| | | | GGCAGGAGGCGGAGCTCAGGCAGTCATGCTCTCTCGCCCACCGCTCA |
| | | | CCTCCTGCCATGCAGCCCAGTTTCTAATAGGCCATTGACAGGTACTG |
| | | | GTCCGCAGCCCTRGGGTTAGGGACCCCTGTTGTAGAGCATATAAAAA |
| | | | CTGAAGAAAGTTTCATAGCATATAAAGATTAGTGCTTGGGGTTTCTG |
| | | | ACAGTGACAAAACAATTTTTTTCCTTTGGAATTTAGGATATACTTCT |
| | | | TATCCTGTCCTTTTTCGCCTCTTGCCCTCAACTCCAATGCATTTTCC |
| | | | TTACATAAATTAAAAGGGACCATCA |
| LNPEP | rs6859160 | 222 | TCTTGAAAATCCTGAGACATATTCAAATACATAGTTTTTACTTACAA |
| Region | | | AAATACTTATAGGCCTGGTGCGGTGGCTCATGCTTGTAATCCCATCA |
| | | | CTTTGGGAGACCGAGGTGGGCAAATTGCCTGAGGTCAGTCTGGCCAA |
| | | | CATGGTGAAAACCCATCTCTACTAAAATACAAAAAAAAAAAAAAAAA |
| | | | AAATTAGCCAGGYCTGGTGGATCACCTGTAATCCCAGCTACTCGGGA |
| | | | GGTTGAGGCAGGAGAATCACTTGAATCCAGGAGGCGGAGTTGCAATG |
| | | | AGCTGAGATCACACCACTGGACTCCAGCCTGGTGACAGAGTGAGACT |
| | | | CTGTCTTAAAACAAAACAAAACAAACAAACAAACAAAAAACATATAA |
| | | | AGATGCTCTTTACTATCCATTTCCA |
| LNPEP | rs6859168 | 223 | TCCTGAGACATATTCAAATACATAGTTTTTACTTACAAAAATACTTA |
| Region | | | TAGGCCTGGTGCGGTGGCTCATGCTTGTAATCCCATCACTTTGGGAG |
| | | | ACCGAGGTGGGCAAATTGCCTGAGGTCAGTCTGGCCAACATGGTTGAA |
| | | | AACCCATCTCTACTAAAATACAAAAAAAAAAAAAAAAAAAATTAGCC |
| | | | AGGTCTGGTGGAKCACCTGTAATCCCAGCTACTCGGGAGGTTGAGGC |
| | | | AGGAGAATCACTTGAATCCAGGAGGCGGAGTTGCAATGAGCTGAGAT |
| | | | CACACCACTGGACTCCAGCCTGGTGACAGAGTGAGACTCTGTCTTAA |
| | | | AACAAAACAAAACAAACAAACAAACAAAAAACATATAAAGATGCTCT |
| | | | TTACTATCCATTTCCATCACCCACC |
| LNPEP | rs6868302 | 224 | CACTTAGACATGGATGTCTATGCATAGACATGGATGTGCAGGAGGTG |
| Region | | | AATGGCACTTCAGAGGACAGGTTCCTGTCAGCCTCTTTGGATTCACG |
| | | | TCCCAGCTCTACAACTTTCAGCCTGGGTGATCTGGAGCAAGTTACTA |
| | | | AATCATTATGTGTTTTTATTGCTTCACCTATAAAATGGCACCTGCTT |
| | | | CATAGAGTGGGCRCAAGTATTAAATTAGATTTTATACGTAAGCATTC |
| | | | AGCACAGTGCCTGGTAAACTGTCAAAAAATGGTGGCCGTTTACATTT |
| | | | TTTCTGCATAAAAGTTTTGAAGGACTTCAGTTAATTCAGAACATAAA |
| | | | AGTGGGTCATGAAATAAAAGTAGCTCTATACTTGGAAGGCAAGAAAA |
| | | | TTTGAATCTAATTCTATTTTTTCTA |
| LNPEP | rs6871162 | 225 | CCTTGGCCGTTCAGTCAGAGGGGTCCATTCGGTCAGTTGAGGGGCCT |
| Region | | | AGAATTTTATTTTTGGTTTACAAAATCATTCCAAGATCCTCTTTAGA |
| | | | GGAAAAATTTATAGAGATTAGTGGGAATGATGAGGAGAACTCAATCT |
| | | | TGAGAGCTCAATCAAAAGGAGATGTTTAAATATCTTTTTAAGTTGGT |
| | | | ATTGGTAAAGTGMTTTGAAGACAGAAAGAATGTAATACATGTCTGGT |
| | | | GTCTGCTTGTCCTATAATTGTCGGAAGGGCCTCAATGATGAAATAAG |
| | | | GGAGGCTGCCATGACACTTGAGTCTTGGTGAGAGGAGCTAGTGTGTC |
| | | | CACATTTATCAAGATCACCTGCAGGAGTTTGGGCTGGCCCCCTCTTA |
| | | | TTAGTAGTTTCTCTGTTTTTTAAAC |
| LNPEP | rs6873441 | 226 | AAAATACTTATAGGCCTGGTGCGGTGGCTCATGCTTGTAATCCCATC |
| Region | | | ACTTTGGGAGACCGAGGTGGGCAAATTGCCTGAGGTCAGTCTGGCCA |
| | | | ACATGGTGAAAACCCATCTCTACTAAAATACAAAAAAAAAAAAAAAA |
| | | | AAAATTAGCCAGGTCTGGTGGATCACCTGTAATCCCAGCTACTCGGG |
| | | | AGGTTGAGGCAGRAGAATCACTTGAATCCAGGAGGCGGAGTTGCAAT |
| | | | GAGCTGAGATCACACCACTGGACTCCAGCCTGGTGACAGAGTGAGAC |
| | | | TCTGTCTTAAAACAAAACAAAACAAACAAACAAACAAAAAACATATA |
| | | | AAGATGCTCTTTACTATCCATTTCCATCACCCACCGTCAGTGGTCCA |
| | | | GACACACTTTCTCCATGCTTCCGCT |
| LNPEP | rs6874656 | 227 | GTGGCTCATGCTTGTAATCCCATCACTTTGGGAGACCGAGGTGGGCA |
| Region | | | AATTGCCTGAGGTCAGTCTCGCCAACATGGTGAAAACCCATCTCTAC |
| | | | TAAAATACAAAAAAAAAAAAAAAAAAAATTAGCCAGGTCTGGTGGAT |
| | | | CACCTGTAATCCCAGCTACTCGGGAGGTTGAGGCAGGAGAATCACTT |
| | | | GAATCCAGGAGGYGGAGTTGCAATGAGCTGAGATCACACCACTGGAC |
| | | | TCCAGCCTGGTGACAGAGTGAGACTCTGTCTTAAAACAAAACAAAAC |
| | | | AAACAAACAAACAAAAAACATATAAAGATGCTCTTTACTATCCATTT |
| | | | CCATCACCCACCGTCAGTGGTCCAGACACACTTTCTCCATGCTTCCG |
| | | | CTTAAGCTTCTCAGCACCAAGTATT |
| LNPEP | rs6879678 | 228 | CTTTGTGGCTTCATCTCCAGCCACACTGGACAGCCACCCCCAGTTTC |
| Region | | | TGCACATGCACTGCTCTCTTGTGTTCCCGGACCAAACTGAGGGTCAG |
| | | | GCTGCTATTTTTTGCTGCCCCAAAACGAGATGCAGATGAACTGGGAA |
| | | | GAGACTTTTTATTTCTATAACCAGTTATATAGGGAGAAGGCCTGGAA |
| | | | ATTATTGCCAGAMCAACTCAAAATTACAAAGTTTTCCAGAGCTTATA |
| | | | TACCTTCTAAACTATATGTTTACGTGTAAGTGTGCATTTCTCTAAAG |
| | | | ACATAAGTGATTAACTTCTTTTAATCCATAACTAAGGTCCGAGTCTT |
| | | | GAAGACCTTCCTCTTGAGCCTCAGTAAATTTACTTAATCTAAATGGG |
| | | | TCCAGGTGCTGGGGTGATTACCCTT |
| LNPEP | rs6887500 | 229 | ATTGGAAGAGGAGAATCAATGATGAAGATGAAAGAAATGTGGAGATT |
| Region | | | GGGGGTAAAGGAAGAAGCTGATAGGCAGAGATTTTAAAAATGGTCAT |
| | | | GCCTTGATCCTTGCAAGTCTTTGGTTCAGAAATGAGCTTCAGTTGGA |
| | | | GAGCAGGACACTGTTGTATGAGGTTGAAGACAGAGTCTAGGTTGGAA |
| | | | GGGGACAGGTAGRTAGGTCTGGTTGGATTAATGGAATTGGGGGCTCA |
| | | | GGGGACAAATGAGTTAAGATTGGCATTTGGGAGCCTTGCCAAGAGAT |
| | | | AGAAAACATTTGCCAGAAATTTAAGCATACTGTCTTTTTTATAGTCA |
| | | | GAAAATTCAGTCACTCGTAAGTTGGGACTGTTCACTTGTCTGAATGT |
| | | | TTTAGATTTAAAGAAAAAATATAGC |
| LNPEP | rs716848 | 230 | AAAGAAAGGAAGGAAGGAAGAAAAAAAAGGAAGGAAGGAAGGAAAAG |
| Region | | | AAAAGAGGGAGGGAGGGAAGGAAGGAAAGAAGGAAGGAAGGAGAAAG |
| | | | AAAAGTAGATCTAACTTATTTTGGGCATGTGTATTAGTTTACTAGTG |
| | | | TTAGCAATGGCAAATCCGTCGGGTCTGCAGAAACTCTATTTTTGCCT |
| | | | TCTTGGAGGAAAKAATTCTGCTGAGGGGCATAAGGCAGAGAGACTGA |
| | | | GGCAACTTTTAGAGCAGGAGTGAAAGTTTATCAAAAAGTTATAGAGC |
| | | | AGGAATGAAAGGAAGTAAAGTACACTTGCAAGAGGGCCAAGTGTACC |
| | | | TGAGAGATCCAAGTGCACTGTTTGGCCCTTGACTTGGGGGTTTTACA |
| | | | CATTGGCATGGTGCCAGGATTTCTG |
| LNPEP | rs7700332 | 231 | ATTGAACCTTTTTCTCCTAGATTTTTCTTTTTTCCCTCTCATTTAGT |
| Region | | | TCTTTTTTAGTCTTGATTTCCCCACGGAGAGTCTCATCTATTCACAT |
| | | | ATTCTCATTTTTTCCTTTTTAAAATACATCTTCCTGCTTAATGATGG |
| | | | GGATACAATTGAAAAATAATAAAACACGTCTTCTTCAGGGATTCTTT |
| | | | TTTATTTATAATRGCTACTCTAAAGACTCACTAAATACAATGCAATA |
| | | | TCTGGACCACCTTAAGATTGCTTTCTAATGATTTTGTTTACTTAGGG |
| | | | TTCACATTTTCTTGTTTCATTAAATGTCTAGTAATTTTTTATTACAT |
| | | | ATTGAATAGTGTCAATGGCACATGGTAGAGATGCTGAATTAAAAAAA |
| | | | ACTCTGTAAAATGTTGATTTTTCTC |
| LNPEP | rs7703341 | 232 | AAAATACCTTGAAATACTTACTGACATTATAGAAATTTAGCCCTTCA |
| Region | | | CTCTGCTGATGTTTATAGTTAAGTGTCAGAAATACTTTTATACAGAA |
| | | | GACCTTGTATGGTTCCTTTGTGTGAGTGGACAGAATTTGTGGAGCAA |
| | | | AGACCTGGAATCCAGCATATGAGAATGTGCAATAATTGTTCAAATGA |
| | | | ATAAGCTTCTCARATTTGGCCTTTGTATAATTAAAATCAGAGTGCTG |
| | | | AAGTGTTGCATATTCCTCATTCTTCTCATTCTTCCAAGTCTCTCTCT |
| | | | CTCTCTCTCTCTATATATATATATACATATATACATATATACATATA |
| | | | TACACATATATACATATATACACATATATACATATATACACATATAT |
| | | | ACATATATACACATATATATACATA |
| LNPEP | rs7713127 | 233 | AGAATACAACTGGATTTTCAGATTTGCTTCTGCATTCAGTCAGTTGT |
| Region | | | AATAGCACAAGTCATGTAGCCTGTGGAAAACTCTGCTGTACACTCAT |
| | | | GAGAGAAGTGGAGTGAAAATGGCATATAACATATTATGATGAAATAG |
| | | | TTTTGACTCTGAAGGCCTCCTGCAAGGGTATCAGGGATTTCTAGGTG |
| | | | TACCCATATCACRTCTTGAGAACATTAATCTTGCGTTTTTCAGGAAC |
| | | | TGGAGAGGAATAGTTTAGGAGTCCACAGAAGGTAGAAAGTGGAGCTG |
| | | | TTGGAATTGGGCAGCAAGTTTCTTAAGATAGATCTAGGTCACAGGAG |
| | | | GGGAATGTTCTGGCCAGGCATATTTGACTGGCCACATTATCAGATGC |
| | | | CTTGGTTATGTGCGGATTCTACCGT |
| LNPEP | rs7716222 | 234 | TTCCATCATCTGGTGATTGCGTTTTCCATTGAGGATTGTTTACATTT |
| Region | | | TCTTGGTCCTTTGTATTTCAAGTAGTTGTGGCTTGCATCCTGGACAT |
| | | | TATGGGTGTTATATTGTGTAGACTCTTTTATCCTCTGAAGAATGTTG |
| | | | ATATTTTTGTTTTGGCTGGCGATCACCCTGCTCAGGTTCAGACCTTA |
| | | | GTTCTGTTTCACMATCTGTGGCCAGTGGCTCCAATGTTAGTTTAGTT |
| | | | CTCCTAGCCTTTGTATGGTAGGCAGAATAATGGTCTCCAAAGATGTC |
| | | | CATTTCCTAATCCCTGAAGCCTTGGTAATATTTTAGGTTACATAATG |
| | | | AAGAGGAGTTAGGTTGCAATTAGAGTTGCGGTTGCTAATCAGCTGAC |
| | | | CTTAAAATAAAGAGGTTATCCTGGA |
| LNPEP | rs7719705 | 235 | AGAGCCACAAAAACAATTCCCAAGCCAATTAAATTCAACTTTTAAAA |
| Region | | | AGGAATTTCCTAATATACCATAGAGTTGGTGAGAAGGCAATGAATGG |
| | | | GTCCCACAAGCTTTCATGTAGCCTTATGGGAAGAGTAAAGGTTAAGC |
| | | | TGTGTCATGGTTGTCAACTGGGCAAAGCCACTGAAAGGCAGGACTCT |
| | | | CTATTAGTTGACRTAACAAAATATTAATAACTAGTGTTATGAATTAG |
| | | | TTGCAGTATGAGCTGAGGTATGAAAGCATGAATTTTAGACCTGACAC |
| | | | TATCCAGGAGGGAAAAAAGTGGATGTTTCTGTACTGATGTTAATCAA |
| | | | AGGTTAAAAATCAAATGACATTTTGAGGAAAACAAACCTAAACAACT |
| | | | CATTAATGGCCACACAACTTAAATT |
| LNPEP | rs7722694 | 236 | AGGCATGTGCTACCATGCCTGGCTAATTTTTATATTTTTAAGTAGAG |
| Region | | | ATGAGGTTTCACCATGTTGGCCAGGCTGGTCTCAAACTCCTGATCTC |
| | | | AAGTGATCCGCCCACCTTGGCCTCCCAAAGTGCTGGGATTTCAGGCG |
| | | | TGAGCCACCTGGCCTGGACTGTAATTGAGGATTTTTCTGTGTCATAT |
| | | | TCTCAACTGTTGYTGGTGTGCTACAGAAAGAGGAGGAAATTTTTTTT |
| | | | AATCTCTGAGGCGAGTAAAGGAAACCAGAATACTACAGGACACCTAA |
| | | | TTTTTTCAATCTTCATGAAAATGCAAGCTGTGAATTTGACGTTTGGT |
| | | | ATCGTGAAGCCAGAGTCTGTACAGATAATTCGCAGCAATTAATGACC |
| | | | ACCCTTCTTAATAATCTTCCATCAG |
| LNPEP | rs7726445 | 237 | GTAATCCTAGCACTTTGGGAGGCCAAGGCAGGAAGATTGCTTGAGGC |
| Region | | | CAGCAGTTCAAGACCAGCCTGGGCAACATAGTGAGAGCCTGTCTCTA |
| | | | CAAAAAAATTAAAAATTAAAAAAAAAAATTAGTCAGGTGTGATGGTA |
| | | | TGCACCTGTGGTCCCAGCTGCTTGAGAGGCTGAGGTGAAAGGATCAC |
| | | | TTGAGCCTGGGCWAAGTCGAAGTGAGCTGTGGTCATGCCACTGCACT |
| | | | GCAGCCTGGGCAAGAGAGTGAGACCCTATCTCAAAAAAAAAAAAAAA |
| | | | AAAAGAGATCAGAAAGGTCTTTTTCTATAGAATGTCCCACACAAGAG |
| | | | ACAGCTTTGCAGGGCCATTTCAAAATAGGTCTAAGAAATATATTTTG |
| | | | GGGTAAAATACCTTTATTTCTTTCA |
| LNPEP | rs7731592 | 238 | GGATTGATCTGCTTTCTCAAGCTTTGCCCCGGGCCTAACCACGTCAG |
| Region | | | CCTGGGACCAGCCCGTGGGGTTTGACTATACCTGGAACAGATGGTTA |
| | | | ATCTATTGGCTTGCTATAATGTAATTTCCATTTGGCTGGCAGTAGGG |
| | | | AAAGGAAGGTACTTCCTGTAAGCTACACACTGATTTTCATCCAGGTG |
| | | | TTCACACATACCRGGTTTTATGAAAGAGAGCTTGACCCTCGCATTCC |
| | | | TGATTAGCATTTTGTTAGTGTGAAAGTAAGGTATAGACACAGAGACA |
| | | | GGTATAATCACAAAATGGTTGGAGTCTTTTATTGTCTCCTTTTCTTA |
| | | | GAGCAAATTTAATAGAGGAGTTTGATTAGCACCTAAGACTTGCTTAA |
| | | | AACTGAGTTACTAATTCTTTTTCCA |
| LNPEP | rs7733312 | 239 | TCCTTCCCTCCCTCCCTCCTTTCCTTTTCCATCCTCCCCCTCACCTT |
| Region | | | TCCTTTTTCTGTAAACTTTTCCATAGCAAATAGAGTAATTCCAAATC |
| | | | ATTTTTTGGAACATTCTTATTAGTGTTCATTCAGCTTCCCTTTCCAC |
| | | | TGAAATGAATTTATTGAGTACTTGGAGTATTTCAAACCCTATGCTTT |
| | | | GTACAGAGAAGASAGTGCTAAATAGGAAACCCTCTCAGTGTTAGAGG |
| | | | GAAAGGAAGATGATGTGGAGGGAAGGAGTCTCTTACTCTGAAGCATT |
| | | | GAACAAAATCAACATTAAAGAGTGAACCAACATTTACCTCCTTTCTTC |
| | | | TTTCATCTTCTTATTTCATAGCTAGAGAGCTGCTGTGCGTTTGAGAC |
| | | | CTAAGTGGTGCAATTAAATCAATTT |
| LNPEP | rs7736466 | 240 | TTTTGTTTTTTACTCCACATGTGTTGATAGAGGTTAATATAAGAAAT |
| Region | | | GTTTGTGTTGGCATAATGCAAAGGTTATTTTTGATTCTGAACCCATA |
| | | | GCAGTTTCTAACCGGTGTTCGTCAGTTTGTGCTTGCTTTTATCCTTG |
| | | | AGGTTAAGGATTGCTCACCAAGCCTTTGATTACTAGGTACATTGCAG |
| | | | AATAAATAAAATSGTTGCTAGTGTATACTCTGTATTAATCTGTCCAC |
| | | | AGCAGCATTGTCAGTGATCTCAAGGTTCTCTGTAGACATTAGTATTG |
| | | | GCTTATGGCATGCTAAAATAGAGATAATTGAGACTATAAAGTTCCAA |
| | | | GTTGAAGTTATCAATAACAACCCTAAAACTATCTTCCTTTTCTTTCC |
| | | | TTCTAAAATAAGACATATGGTAATC |
| LNPEP | rs9127 | 241 | TCATCCCCCACATGTGGCAAGACAAGTTGGCCCTTTCTTACCCAGAG |
| Region | | | GTCTTTTGTGTGACTGCATCTTTCTCCTCCGTTCTCCATTGTGTGCT |
| | | | TTCCATTTTGTCTTTAGTGCCTATACTGTTAGGTGTTTTCTTCACTG |
| | | | GCATTCACAAATTTAAGCCATTGCTGCCTCATTAGCCTTGTATTTTG |
| | | | TGTGCATATCATRTATCCAGACCTGTATGTTCGCTTTAAGCATTCTT |
| | | | ATATCACACTGTCTCCTCATCTACCATATGGTAAATGTTAAAACTCC |
| | | | ACATTTGTCTGCATCAGGGAAAATGCATGGGCACACATCCTCCCTCC |
| | | | CTCCCTCTCTGCTCTCCTCCCTTCCTTCAGGCCTCTTAGCATTGTTT |
| | | | CTTTTCCCATTTCTGATACTACTAC |
| LNPEP | rs9314181 | 242 | TGAAACAGTTGTTATGGAGGCCTGCGTTAGTGAGATCTGGCTTGCCA |
| Region | | | CACTTGTGTTACCCACTCTTTCCAGAGTATACTTTCTTCCCTTCTTC |
| | | | ACCTTTTCAAATACTCATCTTTTTAGGCCCTCTTCAGGTTTTCTGCA |
| | | | TGTTTCCTTATAATATCTTCAACCTCTAGTCAGAATTTGTTTCCTTC |
| | | | CCTTTGTTCCCAYTGCTTTATTTTCATTGTTAGGACATGACTTACAG |
| | | | CCTGATGTAAGTTTCTGTTCATTGTATAAACCTCTGCCTTTCCCAGT |
| | | | TTATTGCAGATCCTTTAGTAACTAGGATTGTAACATATTTATCTTAG |
| | | | TATACTTGGCAGGGTGCCTTGTACAGTAGGTGCTCAGTAACTACTGG |
| | | | ATTGAATTTGTGTTTGTTTTAGGTA |
| AVPR1A | rs1042615 | 243 | AGCAGCGTACTGCTGGCTCTGCACCGGACGCCGCGCAAGACGTCCCG |
| | | | CATGCACCTCTTCATCCGACACCTCAGCCTGGCCGACCTGGCCGTGG |
| | | | CATTCTTCCAGGTGCTGCCGCAAATGTGCTGGGACATCACCTACCGC |
| | | | TTCCGCGGCCCCGACTGGCTGTGCCGCGTGGTGAAGCACCTGCAGGT |
| | | | GTTCGGCATGTTYGCGTCGGCCTACATGCTGGTAGTCATGACAGCCG |
| | | | ACCGCTACATCGCGGTGTGCCACCCGCTCAAGACTCTGCAACAGCCC |
| | | | GCGCGCCGCTCGCGCCTCATGATCGCGGCCGCCTGGGTGCTGAGCTT |
| | | | CGTGCTGAGCACGCCGCAGTACTTCGTCTTCTCCATGATCGAGGTGA |
| | | | ACAATGTCACCAAGGCCCGCGACTG |
| AVPR1A | rs10747983 | 244 | AACTGTGAAAAATAAAATAAGGTGCTGCAACACATTTTTTTCTTGAC |
| | | | TGTAAGCTGTTATTTGGCATAATATCTCAGGTCTTCTCTTTAGTCAA |
| | | | GAAAAGGAAAACTTCCCTTCCCGGAATACTTTTTCAGTTTCTCTTCT |
| | | | TCTGAAACAGACAGGCAGGTAGATTCCTTCCAATCTGAAATATTGTT |
| | | | TTGAGATATGTGRCGTCCATTTCTGGGTACATAACATTGAGAAAATT |
| | | | TAGCAACCAGACAGATGAAACTTCTCAGCCTAAACCGCAGAGAATAA |
| | | | GACCATGTATTTGCCTAGTGCAGAACTAGCACCCAGATCTCATGTTT |
| | | | CCCCAGCCCATTTTCTACTGTCTCATCTCCCAATACATTTAAAAGGA |
| | | | GAAAATACAACTGGGTAGGGTGATA |
| AVPR1A | rs10784339 | 245 | TTGAGATATGTGGCGTCCATTTCTGGGTACATAACATTGAGAAAATT |
| | | | TAGCAACCAGACAGATGAAACTTCTCAGCCTAAACCGCAGAGAATAA |
| | | | GACCATGTATTTGCCTAGTGCAGAACTAGCACCCAGATCTCATGTTT |
| | | | CCCCAGCCCATTTTCTACTGTCTCATCTCCCAATACATTTAAAAGGA |
| | | | GAAAATACAACTSGGTAGGGTGATATGCACTTTTTTTTGTGAGCTGT |
| | | | TCTCAGAAATAACATTCAAATTGAATTGTTTTGCTTGGGGGTACATA |
| | | | TCAACATTTTGAAGCAAGATCTATAGGTTCTGAGGTTCTTACTTTGG |
| | | | AAATGGATTTAGAAAAAAATGGGTTCATCTTAGTTCCAAACCAAAAA |
| | | | GCTTTAGTTTTTGAACTATCAAAGA |
| AVPR1A | rs10877962 | 246 | AACCTCCCAAGTAGCTGGGACTATAGGCACACACCACCATGCCCAGC |
| | | | TAATTTTTTGTATTTTTTTTTTCTTTTTAGAGTAGAGATAGGGGTCT |
| | | | CCCTATGTTGCCCAGGCTGGATTATACATGAATTTTTAAAAATGAAA |
| | | | GTTACACTGAATGTGCCTGCCTGTCCTGCCTCCCCTTTCACCTCCTC |
| | | | CACCCCTTCCACYCGAGACAGCAAGATCAACCCCTCTTCTGCCTCTT |
| | | | CCTCCTCAGTCTACTCAACCTGAAGATCAGGGTGAAGACCTTTATGA |
| | | | TGATCCACTTGCACTTAATGAATAGCAAGCACTTTCTATTATTTTTA |
| | | | AATAACGTTTTCTTTTCTCTAGCTTACTTTATTGTAAGAATACAGTA |
| | | | TATCATATATAATATGCAAAATATG |
| AVPR1A | rs10877969 | 247 | ATATGTATGCATCTGGCCATTCTATGTATCATGTGTCAATCAATCAT |
| | | | CTATCTATCTGTCTATCTATCTATCTATCTATCTATCTATCTATCTA |
| | | | TCATCCATCTATCTGTCTCTCGCTGGTTGTGCTGGATGCCATGGGGC |
| | | | CTGGAAAGCAGGAAAAAAAAATGTTCATTGCAGATTGTAGAACCAGT |
| | | | CCCTTTGTTTAAYCCATATAGTTTTAAACATGTTTTTGACTTAATTT |
| | | | AACTGGTTTTATATACAAAGGAAAGCAGGACTATTACATATGAGGCA |
| | | | CTACTCATATGCCTCACTGGACCTGCTATTAAATTACCCCATAGAGA |
| | | | GTAAAATAATTGTGGTCTTAAAATATGAAAAAGAAAACACAACAGAC |
| | | | AATATTTTATGTGGCACCTTGTGCT |
| AVPR1A | rs10877977 | 248 | TTTACATGTCCATCCCTGTGGGCAGGAAAAGAGTGAAAACAGCCTTA |
| | | | TGTGGATCGCATGAAATGGATTCCTCACAGGAAAGAATGCTCCTGTT |
| | | | GCTAGAAAAGGAGGGTATGCTAAGCTGGCAAAAATAACAGATATTTA |
| | | | CTTCACATATGAAAACCAACCTGTTGATCTCAGACTTGCAATGGATG |
| | | | GCTGAATTTCATYCTAGCCTTTCTTTGCATAAGTGACCGGGGAAGAA |
| | | | GTACTGACTTTACTTTTATCATTTACAGTGATTTTTTTTCTGTATAT |
| | | | GCTAGTTAATTAAACTGAATAAAAGGAATTCCTATATTATGATAATT |
| | | | TAGTCTCAGTAATAGCCAATAAATATTTCTGGAAAGAAGTACCCAGC |
| | | | CCCTGTGTGGGTGCTATTATTGAAT |
| AVPR1A | rs10877986 | 249 | TCCCTGTCTTAGAAGAAAAGTTTTCAACTTTTTACCATTAAGTATGA |
| | | | TGTTAGCTATAGGCTAGTGATCTATGGCCTTTATTGTGTTGAGGTAC |
| | | | ATTCCTTCTATACCTAATTTGTTGAGAATTTTTATCATGAAAGTGTG |
| | | | TTGAATTTTGTCAACTTCTTTTTCTGCATCTATCAAGATGATCGATC |
| | | | ATATGGCTTTTAYTTTTCATTCTGATAATATGGTGAATCATTTTATT |
| | | | GGTTTCTGTATGTGGAACCATCCTAGGCAAGTCAGATTTTGGATTTC |
| | | | CTCCTTTATGTTCCATTCTGTAACATGTTAATGGGAACCGGAATTCA |
| | | | GATCAGAATAACAGCTTAGGAACCAAAGCAGGTATATATATATGTGT |
| | | | GTGTGTGTGTGCGTGTGTGTGTATA |
| AVPR1A | rs11174811 | 250 | TGTAACACTGATCAAAATACGTTTACAATGTCCAAGAGAAAGTCCAG |
| | | | AGTACCTTAAGCAATCCTTTTCTACTCTTTTAATAAAATTTGGCTTT |
| | | | CCTTATACAAATCTGACTTTAAAACAACCTCGCAGTGGGGGAAAAAA |
| | | | GATATTTTTGGCCAGTCAACATTTCCTCTCACCTTCAGCATCTCAGT |
| | | | TTTCATGCTTTTMTTGACCAATAATATGTGAGGAACCAAAAGGAGGC |
| | | | CACTGCCAGTTGTAAAGTTACCATTTTGAAATGCAAGGTGATTGATA |
| | | | GCTTCTAATAGAACTCTAAACTGGCCACAATGAGCAGGAGCTCATTA |
| | | | CACCCCAGGCACTTGTACTCCTAGGAGGTACCATCCCATCTCCTGGA |
| | | | CACTGTTTAAGGCTGCATTTTCTGA |
| AVPR1A | rs11832877 | 251 | TTTTTGAGAATATTTTCTTTTTTTCCAAATTATTACATACTCAGATA |
| | | | TACTCTTGAATCTCTCATTCAACAAGTCCCCAAACTTTGTCCATGAA |
| | | | ACCTTTCTTCTCTTTCTTTTCTCTATACCCCATCATTCTAATTTACA |
| | | | TCACGTTAATCTTTTTGGATTATATTTACATATTTAATTTCTCTTCC |
| | | | ACTTTGCTCCAAYTCAAATTCTTTATAACAACCACAAGAACACGAAA |
| | | | CTCCTGTAACTAGCCAATGTAGTAATTAGGGTAGGATAGGCTATGTC |
| | | | CTGTAGTAACAGAGCAATTCTGACACCTCAGTGGCTTAACAAAAAAA |
| | | | TTATTTCTCACTCATGCAAAGTCTAATGCAAGTTGAGCAGCTTTCCC |
| | | | CCAAGCAGTGACTCTGAGGTCCATG |
| AVPR1A | rs11835545 | 252 | TTTTATTTTGATCTAGATGTCTGAGAGTATGAATGTTCTTAGTGCAA |
| | | | ATAATAAATTGAATGCTCTCGAGGATAAAATTTGAAAATAATTCTAT |
| | | | CTTAAGATGTCTAACAAAATGAATAAAAATTATAAACTCTTATGAAT |
| | | | GAGGTTGTACTCTCCAAGTGTTTCTTGTTAAGAACCATAGAAGGACT |
| | | | TCCCTTTTAGAARTGCTTTGGATATTCTAATACATTTAATGCCAGGG |
| | | | CATAAGCTAGTGGTTGTTAAGCTTTTCTCTCCCTCCCACAGCACCAA |
| | | | GAGACCTAACATTCACCCATTTGTAGCAGTTGTTTTAGAACAGTGAT |
| | | | TGCCAAGGAGGGGAAAAATGAGGAAGCATAGCAGAATTTCTGGGGAA |
| | | | CTGTAGAAAGAGAGAGCATATTGGG |
| AVPR1A | rs11836346 | 253 | TTCCACAGCTTTGTGAACACAGAATAGTCCCATTGAAAAGAAAATCT |
| | | | TTCCGAATTTCATAAATGAATAAGTATCTGATTGTTTTAATGTATTT |
| | | | CGTTAGAAATATTTCATCGTTTTTGTCTCATTACTTACTTAATAATG |
| | | | AGTTAAACATTTTCATAAATGTCTTATAACTTACAAACAGAATCTGG |
| | | | GAGTGCTGAATTRTGATAAAGGAACTGCTCAAGTTAGAAATATTACT |
| | | | TTTACTTTTCTTTGAACTGTTATAAATTATACAGAAAAAATAATACA |
| | | | TGGTATATATGGACCATAAGTAGCAGGAGCTGTAATCCAGGTTTTGC |
| | | | ATACATTATCTTATTTAATCCTCACGAATCTAATGAGATGGATTAAC |
| | | | CACTATTTTACACATAAGGATGCGG |
| AVPR1A | rs16856 | 254 | TGATGCCAACACTATAATTGCCAACCCCATTGAGAAAGGAAAGAAAC |
| | | | ATTTGCCCTGACATCTTCCCTCCAGGCAGGGCTGGCCATGCCACTAG |
| | | | TAGCAAAGAGGAGGGATGTGTTGAGTCATCTAGTAAGTCCCTGTGAA |
| | | | GAGTGGATCCTGGCCCATCTGAACATCTGACCAGAAACTAGTGGCAG |
| | | | CAGTTGTAGAACKTGGTATATGCATGTGCTTCTCTTTTTATGGAATC |
| | | | GGAATCAGGGTGCCCCAGAAAGAAAACGAGCCCAATTTTAAAGGGGT |
| | | | TAATTGGGTATCGTCTTGATTCTTTGTAAGATTGGTTAGGTATTCAG |
| | | | GAATCAGGCTGACCAGGCACAAGTACCTACCAACCTTTGTAAAATAT |
| | | | TCTACACTCTACAATATCATTCACA |
| AVPR1A | rs2030106 | 255 | CACCACCACGCCCGGCTAATTCTTCATATTTTTAGTAGAGACGGGGT |
| | | | TTCACCGTGTTAGCCAGGATGGTCTCAAACTCCTGACCTAAAGTGAT |
| | | | CAGCCAGTCTCGTCCTCCCAAAGTGCTGGGATTACAGGCATGAGCCA |
| | | | CCACACCCAGCAAAGTGGAACAGAATAGACAGCCGTAATGGTTCCAT |
| | | | GTATATTTGGGTRCTTACTATACAATAAAGAGGTCTCCACTAAAACA |
| | | | AGGGAGAAGGATGGCATAAAGGAGTTGGGAAATGCAGAAAATTATGC |
| | | | TAGATTCATCTTCTTATATCACATCTTAGTAGTAGACTCCAAATAAA |
| | | | TTAATGAAGTAAATGTGGAAGGTAAATTACAAACCTGATAGAAGGAA |
| | | | AATGTTATTAGAGAACCTATATGAC |
| AVPR1A | rs2201895 | 256 | GAGCTGCCTAAGGCTGTGGGAGCCCACCTCTTGCATCAATGTGCCCT |
| | | | GGATGTGAGACATGGAGTCAAAGGAGATCATTTTGGAATTTTAATAT |
| | | | TTGACTGCCCTGCTGGAATTTGGACTTGCATGGTGCCTTTAGCCCCT |
| | | | TCATTTTGGCCAATTTCTCCCATTTGGAATGGGTGCATTTATCCAAT |
| | | | GCCTGTACTCCCRTTGTATCGAGGAAGTAACTAACTTCCTTTTGATT |
| | | | TTACAGGCTCATAGGCAGAAGGGACTTGCCTTACCTCAGATGAGACT |
| | | | TTGGACTGTGCACTTTTGAGTTAATGCTGAAATTAGTTAAGACTTTA |
| | | | GGGGACTTTTGGGAAAGCATGATTGGTTTTGAAATGTGAGGACATGA |
| | | | AATTTGGGAGGGGACCAGGGTGGAA |
| AVPR1A | rs3021529 | 257 | TCTTTCCTCTCTTTGAGATTGCCTCTTTCTTACTCCTGAGCACAGGA |
| | | | GCCGGGCGGGTTTTCTGTCCCTTGCCCTGGACAGCACTGCCTGGATG |
| | | | GCCGCTGTCCGGCAGCTGCTCTTTGTCCACCCAAAAAGATGTCCCCA |
| | | | CGACTCAGTAGTAACCAGACGGTCCCCACGGACCACTGCGGCCAAAT |
| | | | TTCCGCCATCCCYGCTGTGGGAATCAGGCTTTTCCCGCAGAAAACCC |
| | | | CAGCAATCTACAGAAAACTCCTTAAGTCCCTAGTCTCCATAGAGAAA |
| | | | ACCAGGAGACACTCCCCCCAAACCCCGCTGTGAATACAGGCACAGCA |
| | | | GCCACTGGGGCTGCAAAGTGATGAGTGCGTTCTTCCCGTCGCAAACA |
| | | | TAGGGTAATAAATAGCATGCATCAA |
| AVPR1A | rs34462214 | 258 | AACATTTCAGTATGAATTTAACTTAAATATTCTTACTGACTATAATA |
| | | | CTAGCGATAATGAAAAATACAATATAAACACTTTATTTTTCCTTTGC |
| | | | TATTTCTTATCTTGCTTGATCTTAGAAGCCTCTTCATATTGTCCATC |
| | | | AAATAAAGAAATTCAGTCTAATTATTGCTTTAGCAGAATTTACACTC |
| | | | AAGTAATAAAAAYTTCAATTGTGCATAGATATGTTGGTAATTTTCAT |
| | | | TCTTTGTGAATACCATCTTACCCATGGCTCCTGATCACCTTTGATAG |
| | | | CAGCATCTTAGCACTAAGTATGATTAAATAATAACCTGTAATTGTTT |
| | | | TCTGGCATAACAAGAGTGAGAAGATCCAAGTTTATATTTAATAATCA |
| | | | AGGAAAAGTCAGTGTTTATTGATTA |
| AVPR1A | rs36014760 | 259 | TGTCTCTTAAAGGGTACTGTCCAATATAAGCCATAACTAAATTAATT |
| | | | AATTCATTATTTGAGTTAGAGTAGCATCTCAGTAACCCAGCACTCGA |
| | | | AGACTGTCAGTCCTTTTAACAACTCTTTGATAGTTCAAAAACTAAAG |
| | | | CTTTTTGGTTTGGAACTAAGATGAACCCATTTTTTTCTAAATCCATT |
| | | | TCCAAAGTAAGAA/- |
| | | | CCTCAGAACCTATAGATCTTGCTTCAAAATGTTGATATGTACCCCCA |
| | | | AGCAAAACAATTCAATTTGAATGTTATTTCTGAGAACAGCTCACAAA |
| | | | AAAAAGTGCATATCACCCTACCCAGTTGTATTTTCTCCTTTTAAATG |
| | | | TATTGGGAGATGAGACAGTAGAAAATGGGCTGGGGAAACATGAGATC |
| | | | TGGGTGCTAGTT |
| AVPR1A | rs7294536 | 260 | TTCCAATTAAAGCAAAATATTCCCAATTTACATATGTGCAATGAGAA |
| | | | GAGTTTTATGGTTAAATATGTTGGAGAAGTGCTGTGTATGCATCCCA |
| | | | CCCTCTCCTGGTGATTTATACATAAAAAGGACCTGAGAAACTTCAGA |
| | | | AAAGAAACTTACTTAACCTTGTTCATCAATGTTTTCCAAGGTTATTT |
| | | | TACCATGGAAACYCCCCATTTTTTTACTTTCCCCATGGAATGGTGAT |
| | | | GAACATGTCACAAGACAAGGTGACAGAGCAGGAGCATCACCATCCTG |
| | | | CCATTTTAAAGTTCACCTTGATCAAAAACCACCTAAATCCAAAGGGC |
| | | | ATCAGCCTAATGGCTAAGGCCAGAATGACCATGAGCCACAAATAACA |
| | | | TCTCTTACCAGAAACATTCCAAACC |
| AVPR1A | rs7302323 | 261 | TTATGCAGTCTTGTAGGACACGTTAAAGATATTGGGCTTGATCTACA |
| | | | AGAAAGGGAAAATGTTGAAGGAATTTTAACAAGGGAAGGGCATAATC |
| | | | ATTTTTGTATCTTTTAAAAGAGAATACTTTGGCTTTATGTGCAAATG |
| | | | AATGGAGGAGGGTGAGAACAGATAGAGACTCAGTTAAGAGACCATAG |
| | | | CAGAGGACCCGAWAAGCTAGAGTATGGTAGGGAAGAAGACATGCAGA |
| | | | GTCATGGTCTTGAGGATGAGTTTGGGAGTATTGGAATAATGXAGTTT |
| | | | ACATCTCTATTGCCGAAATGAGGATTAGTCGTGACCACTCAAGGCAG |
| | | | GAGCCAGCCCCACTTATGAGGGAGAGAAGGCAGCAGAGTCTGGGGAC |
| | | | AGGCTCCTAGACACCTTCTGGATCA |
| AVPR1A | rs7308008 | 262 | AACAACATAAATGAATTTTTTCCAATAGAAATGTAATTGATTTCTGC |
| | | | CCCGTAGGAAAGAAAACTCCAAGCATTATGTTTTATGAACCAATAGA |
| | | | AAAATAATAATCAATCTTACATCTTTTAGCAAAATCATTTCAGAATT |
| | | | CTTGACTGCCTGTGTGTTACTCTTCTTCAGATTCTCCCCTGAACAGG |
| | | | TCTTAACATCTCRTTGGTTCCATCCTTAATTAATAAGCTGAATAAAA |
| | | | CTGTAGCATGTGTTCATTTTACATTTGCAGGAGAGTCATGACTTTAT |
| | | | CTTTATAAAATTTATACATAGCAGCCCTGCGTGGTCTCAGGGGTCTG |
| | | | CCTCTATCTTTGCCACATCCCATGCTCAGGTCCATAGCTATTCTAGC |
| | | | TGGTCCTCACTAGTCCTCTGTTCTA |
| AVPR1A | rs7959001 | 263 | TTCTGGTTAAATGATTTTTAATAAGACGTTAATCTCTTTGTACAATA |
| | | | AGAGTGCTTATACCCTTTTCATAATAATTGTGTAAAGACTTATGATT |
| | | | ACACTAGGCACAGGAAGGTGTTTTCAATAAAACAAAGTGTCCTTCCA |
| | | | GTTCCCTGCTTTGAAGTAGGGTCTTCAATCTTCCCATCTCCATTGTT |
| | | | CAGTGCATATGTWTCACTTAGGATAAGCTAAGTTATGCTAAAGTAAC |
| | | | AAGCAAACAACAAATCTCAGTGGCTTAGAGCAATCAAGATCTATTTC |
| | | | TTATTCATGCTACTATTCATCATGCATAGCTGGGGCTTTACTCCATG |
| | | | TGCTTCTCATTGAGGAACCTAGGTGAGGGGGCTTGATCATCTGGAAT |
| | | | GTCACCAGTCACTGTAGCAGGGAGA |
| AVPR1A | rs7972829 | 264 | AATTATAGATACTGAAATCTGAATTTTATACAATGTTCATGTGTCAG |
| | | | AAATATTCATTTTGATTTTTCTCAATTATTTAAAAATGTAAAAACTA |
| | | | TTCTTAGCTCATAGGACAAACTAAAACATGGATGAGCTAGATTTGGC |
| | | | TTGTGCATCATAGTTTGCCAATTCCTGTTCTAAAGTATGTTAACAAA |
| | | | TCCACATATCTTRAATATTACTATTTTTCATAATAGGTGAGAGCCTA |
| | | | TTTTTAACTCCCGTTATGCTGATAAATAAGCTACTGATTTCACCATT |
| | | | ATGTTAATTAACAAAATATCTATTGTCAATCAGAAGAAAAGGTCACC |
| | | | AATATTCTTATAGTAGTCATCTCTGGTGGGTGGGGCTTTTCTGATAA |
| | | | AATTCTAGCTGCTTCCCCATTCCCT |

An “allele” is defined as any one or more alternative forms of a given gene. In a diploid cell or organism the members of an allelic pair (i.e. the two alleles of a given gene) occupy corresponding positions (loci) on a pair of homologous chromosomes and if these alleles are genetically identical the cell or organism is said to be “homozygous”, but if genetically different the cell or organism is said to be “heterozygous” with respect to the particular gene.

A “gene” is an ordered sequence of nucleotides located in a particular position on a particular chromosome that encodes a specific functional product and may include untranslated and untranscribed sequences in proximity to the coding regions (5′ and 3′ to the coding sequence). Such non-coding sequences may contain regulatory sequences needed for transcription and translation of the sequence or introns etc. or may as yet to have any function attributed to them beyond the occurrence of the SNP of interest.

A “genotype” is defined as the genetic constitution of an organism, usually in respect to one gene or a few genes or a region of a gene relevant to a particular context (i.e. the genetic loci responsible for a particular phenotype).

TABLE 1E
Genotype correlations for SNPs in vasopressin pathway associated
genes with values representing an ability to recover from an
inflammatory condition and an indication of responsiveness to
treatment of an inflammatory condition with a vasopressin
receptor agonist.

| | | Patient | |
|---|---|---|---|
| | | Outcome | Responsiveness |
| POLYMORPHISM | Genotype | Score* | To Treatment\acute{ } |
| rs18059 | TT | 1 | R |
| rs18059 | CT | 1 | R |
| rs18059 | CC | 2 | PR |
| rs27711 | GG | 1 | R |
| rs27711 | AG | 1 | N/A |
| rs27711 | AA | 2 | PR |
| rs38041 | GG | 1 | N/A |
| rs38041 | AG | 1 | N/A |
| rs38041 | AA | 2 | N/A |
| rs10051637 | GG | 1 | PR |
| rs10051637 | AG | 1 | R |
| rs10051637 | AA | 2 | R |
| rs1410713 | AA | 1 | R |
| rs1410713 | AC | 2 | R |
| rs1410713 | CC | 2 | PR |
| rs857240 | CC | 1 | R |
| rs857240 | CT | 2 | PR |
| rs857240 | TT | 2 | N/A |
| rs857242 | CC | 1 | R |
| rs857242 | AC | 2 | PR |
| rs857242 | AA | 2 | N/A |
| rs10877970 | TT | 1 | N/A |
| rs10877970 | CT | 2 | N/A |
| rs10877970 | CC | 2 | N/A |
| rs3803107 | TT | 1 | N/A |
| rs3803107 | CT | 2 | N/A |
| rs3803107 | CC | 2 | N/A |
| rs1495027 | CC | 1 | PR |
| rs1495027 | CT | 2 | R |
| rs1495027 | TT | 2 | R |

*good = 2; poor = 1.
\acute{ }Responsive (R); Poor Response (PR).

A “phenotype” is defined as the observable characters of an organism. In gene association studies, the genetic model at a given locus can change depending on the selection pressures (i.e., the environment), the population studied, or the outcome variable (i.e., the phenotype). For example, the model at rs1410713 changed between the risk of death claims (AA versus AC/CC) and the vasopressin IRP claims (AA/AC versus CC). This is a case of the same outcome variable (survival) following a different genetic model in different environments (i.e., no vasopressin treatment versus vasopressin treatment).

A similar observation would be seen in a gene association study with the hemoblobin, beta gene (HBB) with mortality as the primary outcome variable. A mutation in the HBB gene, which normally produces the beta chain subunit of hemoglobin (B allele), results in an abnormal beta chain called hemoglobin S (S allele; Allison A (1955) Cold Spring Harbor Symp. Quant. Biol. 20:239-255). Hemoglobin S results in abnormal sickle-shaped red blood cells which lead to anemia and other serious complications including death. In the absence of malaria, a gene association study with the HBB gene would suggest a codominant model (survival(BB)>survival (BS)>survival (SS)). However, in the presence of marlaria, a gene association study with the HBB gene would suggest a heterozygote advantage model (survival(BB)<survival(BS)>survival(SS)).

A “single nucleotide polymorphism” (SNP) occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A “transition” is the replacement of one purine by another purine or one pyrimidine by another pyrimidine. A “transversion” is the replacement of a purine by a pyrimidine or vice versa. Single nucleotide polymorphisms can also arise from a deletion (represented by “−” or “del”) of a nucleotide or an insertion (represented by “+” or “ins” or “I”) of a nucleotide relative to a reference allele. Furthermore, a person of skill in the art would appreciate that an insertion or deletion within a given sequence could alter the relative position and therefore the position number of another polymorphism within the sequence. Furthermore, although an insertion or deletion may by some definitions not qualify as a SNP as it may involve the deletion of or insertion of more than a single nucleotide at a given position, as used herein such polymorphisms are also called SNPs as they generally result from an insertion or deletion at a single site within a given sequence.

A “systemic inflammatory response syndrome” or (SIRS) is defined as including both septic (i.e. sepsis or septic shock) and non-septic systemic inflammatory response (i.e. post operative). “SIRS” is further defined according to ACCP (American College of Chest Physicians) guidelines as the presence of two or more of A) temperature >38° C. or <36° C., B) heart rate >90 beats per minute, C) respiratory rate >20 breaths per minute, or PaCO₂<32 mm Hg or the need for mechanical ventilation, and D) white blood cell count >12,000 per mm³ or <4,000 mm³. In the following description, the presence of two, three, or four of the “SIRS” criteria were scored each day over the 28 day observation period.

“Sepsis” is defined as the presence of at least two “SIRS” criteria and known or suspected source of infection. Septic shock was defined as sepsis plus one new organ failure by Brussels criteria plus need for vasopressor medication or vasopressin receptor agonist.

Subject outcome or prognosis as used herein refers the ability of a subject to recover from an inflammatory condition and may be used to determine the efficacy of a treatment regimen, for example the administration of a vasopressin receptor agonist. An inflammatory condition, may be selected from the group consisting of: sepsis, septicemia, pneumonia, septic shock, systemic inflammatory response syndrome (SIRS). Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis. Gram negative sepsis, culture negative sepsis, fungal sepsis, meningococcemia, post-pump syndrome, cardiac stun syndrome, myocardial infarction, stroke, congestive heart failure, hepatitis, epiglottitis, E. coli 0157:H7, malaria, gas gangrene, toxic shock syndrome, pre-eclampsia, eclampsia, HELLP syndrome, mycobacterial tuberculosis, Pneumocystis carinii pneumonia, pneumonia. Leishmaniasis, hemolytic uremic syndrome/thrombotic thrombocytopenic purpura, Dengue hemorrhagic fever, pelvic inflammatory disease, Legionella, Lyme disease. Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis. Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

Assessing subject outcome, prognosis, or response of a subject to vasopressin receptor agonist administration may be accomplished by various methods. For Example, an “APACHE II” score is defined as Acute Physiology And Chronic Health Evaluation and herein was calculated on a daily basis from raw clinical and laboratory variables. Vincent et al. (Vincent J L. Ferreira F. Moreno R. 2000 Crit. Care Clin. 16:353-366) summarize APACHE score as follows “First developed in 1981 by Kuans et al. the APACHE score has become the most commonly used survival prediction model in ICUs worldwide. The APACHE II score, a revised and simplified version of the original prototype, uses a point score based on initial values of 12 routine physiologic measures, age, and previous health status to provide a general measure of severity of disease. The values recorded are the worst values taken during the subject's first 24 hours in the ICU. The score is applied to one of 34 admission diagnoses to estimate a disease-specific probability of mortality (APACHE II predicted risk of death). The maximum possible APACHE II score is 71, and high scores have been well correlated with mortality. The APACHE II score has been widely used to stratify and compare various groups of critically ill subjects, including subjects with sepsis, by severity of illness on entry into clinical trials”.

A “Brussels score” score is a method for evaluating organ dysfunction as compared to a baseline. If the Brussels score is 0 (i.e. moderate, severe, or extreme), then organ failure was recorded as present on that particular day (see TABLE 2A below). In the following description, to correct for deaths during the observation period, days alive and free of organ failure (DAF) were calculated as previously described. For example, acute lung injury was calculated as follows. Acute lung injury is defined as present when a subject meets all of these four criteria. 1) Need for mechanical ventilation. 2) Bilateral pulmonary infiltrates on chest X-ray consistent with acute lung injury. 3) PaO₂/FiO₂ ratio is less than 300 mmHg, 4) No clinical evidence of congestive heart failure or if a pulmonary artery catheter is in place for clinical purposes, a pulmonary capillary wedge pressure less than 18 mm Hg (1). The severity of acute lung injury is assessed by measuring days alive and free of acute lung injury over a 28-day observation period. Acute lung injury is recorded as present on each day that the person has moderate, severe or extreme dysfunction as defined in the Brussels score. Days alive and free of acute lung injury is calculated as the number of days after onset of acute lung injury that a subject is alive and free of acute lung injury over a defined observation period (28 days). Thus, a lower score for days alive and free of acute lung injury indicates more severe acute lung injury. The reason that days alive and free of acute lung injury is preferable to simply presence or absence of acute lung injury, is that acute lung injury has a high acute mortality and early death (within 28 days) precludes calculation of the presence or absence of acute lung injury in dead subjects. The cardiovascular, renal, neurologic, hepatic and coagulation dysfunction were similarly defined as present on each day that the person had moderate, severe or extreme dysfunction as defined by the Brussels score. Days alive and free of steroids are days that a person is alive and is not being treated with exogenous corticosteroids (e.g. hydrocortisone, prednisone, methylprednisolone). Days alive and free of pressors are days that a person is alive and not being treated with intravenous vasopressors (e.g. dopamine, norepinephrine, epinephrine or phenylephrine). Days alive and free of an International Normalized Ratio (INR)>1.5 are days that a person is alive and does not have an INR>1.5.

TABLE 2A
Brussels Organ Dysfunction Scoring System

| | ORGANS | | | | |
|---|---|---|---|---|---|
| | Free of Organ | | Clinically Significant | | |
| | Dysfunction | | Organ Dysfunction | | |
| | Normal | Mild | Moderate | Severe | Extreme |
| | DAF ORGAN DYSFUNCTION SCORE | | | | |
| | 1 | | 0 | | |
| Cardiovascular | >90 | ≦90 | ≦90 | ≦90 plus | ≦90 plus |
| Systolic BP | | Responsive | Unresponsive to | pH ≦ 7.3 | pH ≦ 7.2 |
| (mmHg) | | to fluid | fluid | | |
| Pulmonary | >400 | 400-301 | 300-201 | 200-101 | ≦100 |
| Pₐo₂/Fₗo₂ (mmHg) | | | Acute lung injury | ARDS | Severe ARDS |
| Renal | <1.5 | 1.5-1.9 | 2.0-3.4 | 3.5-4.9 | ≧5.0 |
| Creatinine | | | | | |
| (mg/Dl) | | | | | |
| Hepatic | <1.2 | 1.2-1.9 | 2.0-5.9 | 6.0-11.9 | ≧12 |
| Bilirubin (mg/dL) | | | | | |
| Hematologic | >120 | 120-81 | 80-51 | 50-21 | ≦20 |
| Platelets | | | | | |
| (×10⁵/mm³) | | | | | |
| Neurologic | 15 | 14-13 | 12-10 | 9-6 | ≦5 |
| (Glascow Score) | | | | | |

Round Table Conference on Clinical Trials for the Treatment of Sepsis Brussels, Mar. 12-14, 1994.

### 2. General Methods

One aspect of the invention may involve the identification of subjects or the selection of subjects that are either at risk of developing and inflammatory condition or the identification of subjects who already have an inflammatory condition. For example, subjects who have undergone major surgery or scheduled for or contemplating major surgery may be considered as being at risk of developing an inflammatory condition. Furthermore, subjects may be determined as having an inflammatory condition using diagnostic methods and clinical evaluations known in the medical arts. An inflammatory condition, may be selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulonephritis, interstitial-nephritis, acute tubular necrosis (ATN), subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection. Gram positive sepsis. Gram negative sepsis, culture negative sepsis, fungal sepsis, meningococcemia, post-pump syndrome, cardiac stun syndrome, myocardial infarction, stroke, congestive heart failure, hepatitis, epiglottitis, E. coli 0157:H7, malaria, gas gangrene, toxic shock syndrome, pre-eclampsia, eclampsia, HELLP syndrome, mycobacterial tuberculosis, Pneumocystis carinii pneumonia, pneumonia. Leishmaniasis, hemolytic uremic syndrome/thrombotic thrombocytopenic purpura. Dengue hemorrhagic fever, pelvic inflammatory disease, Legionella, Lyme disease, Influenza A, Epstein-Barr virus, encephalitis, inflammatory diseases and autoimmunity including rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

Once a subject is identified as being at risk for developing or having an inflammatory condition or is to be administered vasopressin receptor agonist, then genetic sequence information may be obtained from the subject. Or alternatively genetic sequence information may already have been obtained from the subject. For example, a subject may have already provided a biological sample for other purposes or may have even had their genetic sequence determined in whole or in part and stored for future use. Genetic sequence information may be obtained in numerous different ways and may involve the collection of a biological sample that contains genetic material, particularly, genetic material containing the sequence or sequences of interest. Many methods are known in the art for collecting biological samples and extracting genetic material from those samples. Genetic material can be extracted from blood, tissue, hair and other biological material. There are many methods known to isolate DNA and RNA from biological material. Typically. DNA may be isolated from a biological sample when first the sample is lysed and then the DNA is separated from the lysate according to any one of a variety of multi-step protocols, which can take varying lengths of time. DNA isolation methods may involve the use of phenol (Sambrook. J. et al., “Molecular Cloning”, Vol. 2, pp. 9.14-9.23. Cold Spring Harbor Laboratory Press (1989) and Ausubel. Frederick M. et al. “Current Protocols in Molecular Biology”, Vol. 1, pp. 2.2.1-2.4.5, John Wiley & Sons. Inc. (1994)). Typically, a biological sample is lysed in a detergent solution and the protein component of the lysate is digested with proteinase for 12-18 hours. Next, the lysate is extracted with phenol to remove most of the cellular components, and the remaining aqueous phase is processed further to isolate DNA. In another method, described in Van Ness et al. (U.S. Pat. No. 5,130,423), non-corrosive phenol derivatives are used for the isolation of nucleic acids. The resulting preparation is a mix of RNA and DNA.

Other methods for DNA isolation utilize non-corrosive chaotropic agents. These methods, which are based on the use of guanidine salts, urea and sodium iodide, involve lysis of a biological sample in a chaotropic aqueous solution and subsequent precipitation of the crude DNA fraction with a lower alcohol. The final purification of the precipitated, crude DNA fraction can be achieved by any one of several methods, including column chromatography (Analects, (1994) Vol 22. No. 4. Pharmacia Biotech), or exposure of the crude DNA to a polyanion-containing protein as described in Koller (U.S. Pat. No. 5,128,247)

Yet another method of DNA isolation, which is described by Botwell, D. D. L. (Anal. Biochem. (1987) 162:463-465) involves lysing cells in 6M guanidine hydrochloride, precipitating DNA from the lysate at acid pH by adding 2.5 volumes of ethanol, and washing the DNA with ethanol.

Numerous other methods are known in the art to isolate both RNA and DNA, such as the one described by CHOMCZYNSKI (U.S. Pat. No. 5,945,515), whereby genetic material can be extracted efficiently in as little as twenty minutes. EVANS and HUGH (U.S. Pat. No. 5,989,431) describe methods for isolating DNA using a hollow membrane filter.

Once a subject's genetic material has been obtained from the subject it may then be further be amplified by Reverse Transcription Polymerase Chain Reaction (RT-PCR). Polymerase Chain Reaction (PCR), Transcription Mediated Amplification (TMA). Ligase chain reaction (LCR). Nucleic Acid Sequence Based Amplification (NASBA) or other methods known in the art, and then further analyzed to detect or determine the presence or absence of one or more polymorphisms or mutations in the sequence of interest, provided that the genetic material obtained contains the sequence of interest. Particularly, a person may be interested in determining the presence or absence of a mutation in a vasopressin pathway associated gene sequence, as described in TABLES 1A-D. The sequence of interest may also include other mutations, or may also contain some of the sequence surrounding the mutation of interest.

Detection or determination of a nucleotide identity, or the presence of one or more single nucleotide polymorphism(s) (SNP typing), may be accomplished by any one of a number methods or assays known in the art. Many DNA typing methodologies are useful for use in the detection of SNPs. The majority of SNP genotyping reactions or assays can be assigned to one of four broad groups (sequence-specific hybridization, primer extension, oligonucleotide ligation and invasive cleavage). Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, etc.

In general, sequence-specific hybridization involves a hybridization probe, which is capable of distinguishing between two DNA targets differing at one nucleotide position by hybridization. Usually probes are designed with the polymorphic base in a central position in the probe sequence, whereby under optimized assay conditions only the perfectly matched probe target hybrids are stable and hybrids with a one base mismatch are unstable. A strategy which couples detection and sequence discrimination is the use of a “molecular beacon”, whereby the hybridization probe (molecular beacon) has 3′ and 5′ reporter and quencher molecules and 3′ and 5′ sequences which are complementary such that absent an adequate binding target for the intervening sequence the probe will form a hairpin loop. The hairpin loop keeps the reporter and quencher in close proximity resulting in quenching of the fluorophor (reporter) which reduces fluorescence emissions. However, when the molecular beacon hybridizes to the target the fluorophor and the quencher are sufficiently separated to allow fluorescence to be emitted from the fluorophor.

Similarly, primer extension reactions (i.e. mini sequencing, nucleotide-specific extensions, or simple PCR amplification) are useful in sequence discrimination reactions. For example, in mini sequencing a primer anneals to its target DNA immediately upstream of the SNP and is extended with a single nucleotide complementary to the polymorphic site. Where the nucleotide is not complementary, no extension occurs.

Oligonucleotide ligation assays require two sequence-specific probes and one common ligation probe per SNP. The common ligation probe hybridizes adjacent to a sequence-specific probe and when there is a perfect match of the appropriate sequence-specific probe, the ligase joins both the sequence-specific and the common probes. Where there is not a perfect match the ligase is unable to join the sequence-specific and common probes. Probes used in hybridization can include double-stranded DNA, single-stranded DNA and RNA oligonucleotides, and peptide nucleic acids. Hybridization methods for the identification of single nucleotide polymorphisms or other mutations involving a few nucleotides are described in the U.S. Pat. Nos. 6,270,961; 6,025,136; and 6,872,530. Suitable hybridization probes for use in accordance with the invention include oligonucleotides and PNAs from about 10 to about 400 nucleotides, alternatively from about 20 to about 200 nucleotides, or from about 30 to about 100 nucleotides in length.

Alternatively, an invasive cleavage method requires an oligonucleotide called an Invader™ probe and sequence-specific probes to anneal to the target DNA with an overlap of one nucleotide. When the sequence-specific probe is complementary to the polymorphic base, overlaps of the 3′ end of the invader oligonucleotide form a structure that is recognized and cleaved by a Flap endonuclease releasing the 5′ arm of the allele specific probe.

5′ exonuclease activity or TaqMan™ assay (Applied Biosystems) is based on the 5′ nuclease activity of Taq polymerase that displaces and cleaves the oligonucleotide probes hybridized to the target DNA generating a fluorescent signal. It is necessary to have two probes that differ at the polymorphic site wherein one probe is complementary to the ‘normal’ sequence and the other to the mutation of interest. These probes have different fluorescent dyes attached to the 5′ end and a quencher attached to the 3′ end when the probes are intact the quencher interacts with the fluorophor by fluorescence resonance energy transfer (FRET) to quench the fluorescence of the probe. During the PCR annealing step the hybridization probes hybridize to target DNA. In the extension step the 5′ fluorescent dye is cleaved by the 5′ nuclease activity of Taq polymerase, leading to an increase in fluorescence of the reporter dye. Mismatched probes are displaced without fragmentation. The presence of a mutation in a sample is determined by measuring the signal intensity of the two different dyes.

The Illumina Golden Gate™ Assay uses a combined oligonucleotide ligation assay/allele-specific hybridization approach (SHEN R et al Mutat Res 2005573:70-82). The first series of steps involve the hybridization of three oligonucleotides to a set of specific target SNPs; two of these are fluorescently-labelled allele-specific oligonucleotides (ASOs) and the third a locus-specific oligonucleotide (LSO) binding 1-20 bp downstream of the ASOs. A second series of steps involve the use of a stringent polymerase with high 3′ specificity that extends only oligonucleotides specifically matching an allele at a target SNP. The polymerase extends until it reaches the LSO Locus-specificity is ensured by requiring the hybridization of both the ASO and LSO in order that extension can proceed. After PCR amplification with universal primers, these allele-specific oligonucleotide extension products are hybridized to an array which has multiple discretely tagged addresses (in this case 1536 addresses) which match an address embedded in each LSO. Fluorescent signals produced by each hybridization product are detected by a bead array reader from which genotypes at each SNP locus may be ascertained.

It will be appreciated that numerous other methods for sequence discrimination and detection are known in the art and some of which are described in further detail below. It will also be appreciated that reactions such as arrayed primer extension mini sequencing, tag microarrays and sequence-specific extension could be performed on a microarray. One such array based genotyping platform is the microsphere based tag-it high throughput genotyping array (BORTOLIN S. et al. Clinical Chemistry (2004) 50(11): 2028-36). This method amplifies genomic DNA by PCR followed by sequence-specific primer extension with universally tagged genotyping primers. The products are then sorted on a Tag-It array and detected using the Luminex xMAP system.

Mutation detection methods may include but are not limited to the following:

Restriction Fragment Length Polymorphism (RFLP) strategy—An RFLP gel-based analysis can be used to indicate the presence or absence of a specific mutation at polymorphic sites within a gene. Briefly, a short segment of DNA (typically several hundred base pairs) is amplified by PCR. Where possible, a specific restriction endonuclease is chosen that cuts the short DNA segment when one polymorphism is present but does not cut the short DNA segment when the polymorphism is not present, or vice versa. After incubation of the PCR amplified DNA with this restriction endonuclease, the reaction products are then separated using gel electrophoresis. Thus, when the gel is examined the appearance of two lower molecular weight bands (lower molecular weight molecules travel farther down the gel during electrophoresis) indicates that the DNA sample had a polymorphism was present that permitted cleavage by the specific restriction endonuclease. In contrast, if only one higher molecular weight band is observed (at the molecular weight of the PCR product) then the initial DNA sample had the polymorphism that could not be cleaved by the chosen restriction endonuclease. Finally, if both the higher molecular weight band and the two lower molecular weight bands are visible then the DNA sample contained both polymorphisms, and therefore the DNA sample, and by extension the subject providing the DNA sample, was heterozygous for this polymorphism;

For example the Maxam-Gilbert technique for sequencing (MAXAM A M, and GILBERT W. Proc. Natl. Acad. Sci. USA (1977) 74(4):560-564) involves the specific chemical cleavage of terminally labelled DNA. In this technique four samples of the same labeled DNA are each subjected to a different chemical reaction to effect preferential cleavage of the DNA molecule at one or two nucleotides of a specific base identity. The conditions are adjusted to obtain only partial cleavage, DNA fragments are thus generated in each sample whose lengths are dependent upon the position within the DNA base sequence of the nucleotide(s) which are subject to such cleavage. After partial cleavage is performed, each sample contains DNA fragments of different lengths, each of which ends with the same one or two of the four nucleotides. In particular, in one sample each fragment ends with a C, in another sample each fragment ends with a C or a T, in a third sample each ends with a G, and in a fourth sample each ends with an A or a G. When the products of these four reactions are resolved by size, by electrophoresis on a polyacrylamide gel, the DNA sequence can be read from the pattern of radioactive bands. This technique permits the sequencing of at least 100 bases from the point of labeling. Another method is the dideoxy method of sequencing was published by SANGER et al. (Proc. Natl. Acad. Sci. USA (1977) 74(12):5463-5467). The Sanger method relies on enzymatic activity of a DNA polymerase to synthesize sequence-dependent fragments of various lengths. The lengths of the fragments are determined by the random incorporation of dideoxynucleotide base-specific terminators. These fragments can then be separated in a gel as in the Maxam-Gilbert procedure, visualized, and the sequence determined. Numerous improvements have been made to refine the above methods and to automate the sequencing procedures. Similarly, RNA sequencing methods are also known. For example, reverse transcriptase with dideoxynucleotides have been used to sequence encephalomyocarditis virus RNA (ZIMMERN D. and KAESBERG P. Proc. Natl. Acad. Sci. USA (1978) 75(9):4257-4261). MILLS D R. and KRAMER F R. (Proc. Natl. Acad. Sci. USA (1979) 76(5):2232-2235) describe the use of Qβ replicase and the nucleotide analog inosine for sequencing RNA in a chain-termination mechanism. Direct chemical methods for sequencing RNA are also known (PEATTIE D A. Proc. Natl. Acad. Sci. USA (1979) 76(4): 1760-1764). Other methods include those of Donis-Keller et al. (1977. Nucl. Acids Res. 4:2527-2538). SIMONCSITS A. et al. (Nature (1977) 269(5631):833-836), AXELROD V D. et al. (Nucl. Acids Res. (1978) 5(10):3549-3563), and KRAMER F R. and MILLS D R. (Proc. Natl. Acad. Sci. USA (1978) 75(11):5334-5338). Nucleic acid sequences can also be read by stimulating the natural fluoresce of a cleaved nucleotide with a laser while the single nucleotide is contained in a fluorescence enhancing matrix (U.S. Pat. No. 5,674,743); In a mini sequencing reaction, a primer that anneals to target DNA adjacent to a SNP is extended by DNA polymerase with a single nucleotide that is complementary to the polymorphic site. This method is based on the high accuracy of nucleotide incorporation by DNA polymerases. There are different technologies for analyzing the primer extension products. For example, the use of labeled or unlabeled nucleotides, ddNTP combined with dNTP or only ddNTP in the mini sequencing reaction depends on the method chosen for detecting the products;

Probes used in hybridization can include double-stranded DNA, single-stranded DNA and RNA oligonucleotides, and peptide nucleic acids. Hybridization methods for the identification of single nucleotide polymorphisms or other mutations involving a few nucleotides are described in the U.S. Pat. Nos. 6,270,961; 6,025,136; and 6,872,530. Suitable hybridization probes for use in accordance with the invention include oligonucleotides and PNAs from about 10 to about 400 nucleotides, alternatively from about 20 to about 200 nucleotides, or from about 30 to about 100 nucleotides in length.

A template-directed dye-terminator incorporation with fluorescent polarization-detection (TDI-FP) method is described by FREEMAN B D. et al. (J Mol Diagnostics (2002) 4(4):209-215) for large scale screening;

Oligonucleotide ligation assay (OLA) is based on ligation of probe and detector oligonucleotides annealed to a polymerase chain reaction amplicon strand with detection by an enzyme immunoassay (VILLAHERMOSA M L. J Hum Virol (2001) 4(5):238-48; ROMPPANEN E L. Scand J Clin Lab Invest (2001) 61 (2): 123-9; IANNONE M A. et al. Cytometry (2000) 39(2): 131-40);

Ligation-Rolling Circle Amplification (L-RCA) has also been successfully used for genotyping single nucleotide polymorphisms as described in QI X. et al. Nucleic Acids Res (2001) 29(22):E116;

5′ nuclease assay has also been successfully used for genotyping single nucleotide polymorphisms (AYDIN A. et al. Biotechniques (2001) (4):920-2, 924, 926-8.);

Polymerase proofreading methods are used to determine SNPs identities, as described in WO 0181631:

Detection of single base pair DNA mutations by enzyme-amplified electronic transduction is described in PATOLSKY F et al. Nat. Biotech. (2001) 19(3):253-257;

Gene chip technologies are also known for single nucleotide polymorphism discrimination whereby numerous polymorphisms may be tested for simultaneously on a single array (EP 1120646 and GILLES P N. et al. Nat. Biotechnology (1999) 17(4):365-70);

Matrix assisted laser desorption ionization time of flight (MALDI-TOF) mass spectroscopy is also useful in the genotyping single nucleotide polymorphisms through the analysis of microsequencing products (HAFF L A. and SMIRNOV I P. Nucleic Acids Res. (1997) 25(18):3749-50; HAFF L A. and SMIRNOV I P. Genome Res. (1997) 7:378-388; SUN X. et al. Nucleic Acids Res. (2000) 28 e68; BRAUN A. et al. Clin. Chem. (1997) 43:1151-1158: LITTLE D P. et al. Eur. J. Clin. Chem. Clin. Biochem. (1997) 35:545-548; FEI Z. et al. Nucleic Acids Res. (2000) 26:2827-2828; and BLONDAL T. et al. Nucleic Acids Res. (2003) 31(24):e155).

Sequence-specific PCR methods have also been successfully used for genotyping single nucleotide polymorphisms (HAWKINS J R. et al. Hum Mutat (2002) 19(5):543-553). Alternatively, a Single-Stranded Conformational Polymorphism (SSCP) assay or a Cleavase Fragment Length Polymorphism (CFLP) assay may be used to detect mutations as described herein.

Alternatively, if a subject's sequence data is already known, then obtaining may involve retrieval of the subjects nucleic acid sequence data (for example from a database), followed by determining or detecting the identity of a nucleic acid or genotype at a polymorphic site by reading the subject's nucleic acid sequence at the one or more polymorphic sites.

Once the identity of a polymorphism(s) is determined or detected an indication may be obtained as to subject response to vasopressin receptor agonist administration based on the genotype (the nucleotide at the position) of the polymorphism of interest. In the present invention, polymorphisms in vasopressin pathway associated gene sequences, are used to predict a subject's response to vasopressin receptor agonist treatment. Methods for predicting a subject's response to vasopressin receptor agonist treatment may be useful in making decisions regarding the administration of vasopressin receptor agonist.

Methods of treatment of an inflammatory condition in a subject having an improved response genotype in a vasopressin pathway associated gene are described herein. An improved response may include an improvement subsequent to administration of said therapeutic agent, whereby the subject has an increased likelihood of survival, reduced likelihood of organ damage or organ dysfunction (Brussels score), an improved APACHE II score, days alive and free of pressors, inotropes, and reduced systemic dysfunction (cardiovascular, respiratory, ventilation, central nervous system, coagulation |INR>1.5|, renal and/or hepatic).

As described above genetic sequence information or genotype information may be obtained from a subject wherein the sequence information contains one or more polymorphic sites in a vasopressin pathway associated gene sequence. Also, as previously described the sequence identity of one or more polymorphisms in a vasopressin pathway associated gene sequence of one or more subjects may then be detected or determined. Furthermore, subject response to administration of vasopressin receptor agonist may be assessed as described above. For example, the APACHE II scoring system or the Brussels score may be used to assess a subject's response to treatment by comparing subject scores before and after treatment. Once subject response has been assessed, subject response may be correlated with the sequence identity of one or more polymorphism(s). The correlation of subject response may further include statistical analysis of subject outcome scores and polymorphism(s) for a number of subjects.

Methods of treatment of an inflammatory condition in a subject having one or more of the risk genotypes in AVP, AVPR1A LNPEP or LRAP (or a SNP in linkage disequilibrium thereto) associated with improved response to a therapeutic agent are described herein. An improved response may include an improvement subsequent to administration of said therapeutic agent, whereby the subject has an increased likelihood of survival, reduced likelihood of organ damage or organ dysfunction (Brussels score), an improved APACHE II score, days alive and free of pressors, inotropes, and reduced systemic dysfunction (cardiovascular, respiratory, ventilation, central nervous system, coagulation |INR>1.5|, renal and/or hepatic).

As described above genetic sequence information or genotype information may be obtained from a subject wherein the sequence information contains one or more single nucleotide polymorphic sites in AVP. AVPR1A LNPEP or LRAP sequences. Also, as previously described the sequence identity of one or more single nucleotide polymorphisms in the AVP, AVPR1A or LNPEP sequences of one or more subjects may then be detected or determined. Furthermore, subject outcome or prognosis may be assessed as described above, for example the APACHE II scoring system or the Brussels score may be used to assess subject outcome or prognosis by comparing subject scores before and after treatment. Once subject outcome or prognosis has been assessed, subject outcome or prognosis may be correlated with the sequence identity of one or more single nucleotide polymorphism(s). The correlation of subject outcome or prognosis may further include statistical analysis of subject outcome scores and polymorphism(s) for a number of subjects.

### 3. Analytical Methods

### Patient Cohort Selection

a. Intensive Care Unit (ICU) Cohort Inclusion Criteria

All subjects admitted to the ICU of St. Paul's Hospital (SPH) were screened for study inclusion. SPH ICU is a mixed medical-surgical ICU in a tertiary care, university-affiliated teaching hospital. Subjects were included in the study if they met at least two out of four SIRS criteria: 1) fever (>38° C.) or hypothermia (<36° C.), 2) tachycardia (>90 beats/minute), 3) tachypnea (>20 breaths/minute), PaCO₂<32 mm Hg, or need for mechanical ventilation, and 4) leukocytosis (total leukocyte count >12,000 mm³) or leukopenia (<4,000 mm³). Subjects were included in the analysis if they met the diagnostic criteria for septic shock (sepsis and cardiovascular dysfunction (as defined by Brussels scoring system) and one other organ dysfunction) on admission to the ICU. Subjects were excluded if blood could not be obtained for genotype analysis. Baseline characteristics (age, gender, admission APACHE II score (KNAUS W A. et al. Crit. Care Med. (1985) 13:818-829), together with medical vs. surgical diagnosis KNAUS W A. et al. Chest (1991) 100:1619-1636.) were recorded on admission to the ICU. The full cohort meeting these criteria included 1072 Caucasian subjects and 153 Asian subjects.

The Institutional Review Board at Providence Health Care and the University of British Columbia approved this study.

b. Biological Plausibility (BP) Cohort Inclusion Criteria

An independent cohort of Caucasian subjects (N=102) scheduled for first time elective coronary artery bypass grafting that required cardiopulmonary bypass is referred to as the “Biological Plausibility” (BP) cohort. Significant SNP-biomarker associations identified in this cohort may provide insight into biological processes underlying SNP-phenotype associations observed in the ICU cohort or subsets of the ICU cohort.

For the BP cohort, individuals were included in the analysis if they were met diagnostic criteria for systemic inflammatory response syndrome (SIRS). Subjects were excluded from the study if they had undergone 1) urgent or emergency cardiopulmonary bypass surgery or 2) valve or repeat cardiac surgery. Subjects with urgent or emergency cardiopulmonary bypass surgery were excluded because they may have had an inflammatory response due to other triggers (i.e. shock). Subjects with valve surgery or repeat surgery were excluded because they could have had different pre-operative pathophysiology or longer total surgical and cardiopulmonary bypass time than subjects having elective cardiopulmonary bypass surgery.

The Institutional Review Board at Providence Health Care and the University of British Columbia approved this study.

### Clinical Phenotype

The primary outcome variable evaluated in this study was 28-day mortality. Various organ dysfunctions were considered as secondary outcome variables. Baseline demographics recorded were age, gender, admission APACHE II score (KNAUS W A. et al. Crit. Care Med (1985) 13:818-829), and medical or surgical diagnosis on admission to the ICU (based on the APACHE III diagnostic codes) (KNAUS W A. et al. Chest (1991) 100:1619-1636) (TABLE 2B).

TABLE 2B
Baseline characteristics key.
Baseline Key

| | |
|---|---|
| AGE | Given In Years |
| GENDER | Percentage of Male Subjects |
| APACHE II | APACHE II score |
| % SURGICAL | The % of Subjects with a SURGICAL ICU |
| | admitting diagnosis |
| SEP. ADMIT | Sepsis upon admission |
| SEP. ANY | Sepsis anytime during admission |
| SS. ADMIT | Septic shock upon admission |
| SS. ANY | Septic shock anytime during admission |

After meeting the inclusion criteria, data were recorded for each 24-hour period (8 am to 8 am) for 28-days after ICU admission or until hospital discharge to evaluate organ dysfunction, the intensity of SIRS (Systemic Inflammatory Response Syndrome) and sepsis. Raw clinical and laboratory variables were recorded using the worst or most abnormal variable for each 24-hour period with the exception of Glasgow Coma Score, for which the best possible score for each 24-hour period was recorded. Missing data on the date of admission was assigned a normal value and missing data after day one was substituted by carrying forward the value from the previous day. When data collection for each patient was complete, all patient identifiers were removed from all records and the patient file was assigned a unique random number linked with the blood samples. The completed raw data file was used to calculate descriptive and severity of illness scores using standard definitions as described below.

Organ dysfunction was first evaluated at baseline and then daily using the Brussels score (SIBBALD W J. and VINCENT J L. Chest (1995) 107(2):522-7) (see TABLE 2A in General Methods Section). If the Brussels score was moderate, severe, or extreme dysfunction then organ dysfunction was recorded as present on that day. To correct for deaths during the observation period, we calculated the days alive and free of organ dysfunction (RUSSELL J A. et al. Crit. Care Med (2000) 28(10):3405-11 and BERNARD G R. et al. Chest (1997) 112(1): 164-72) (TABLE 2C). For example, the severity of cardiovascular dysfunction was assessed by measuring days alive and free of cardiovascular dysfunction over a 28-day observation period. Days alive and free of cardiovascular dysfunction was calculated as the number of days after inclusion that a patient was alive and free of cardiovascular dysfunction over 28-days. Thus, a lower score for days alive and free of cardiovascular dysfunction indicates more cardiovascular dysfunction. The reason that days alive and free of cardiovascular dysfunction is preferable to simply presence or absence of cardiovascular dysfunction is that severe sepsis has a high acute mortality so that early death (within 28-days) precludes calculation of the presence or absence of cardiovascular dysfunction in dead subjects. Organ dysfunction has been evaluated in this way in observational studies (Russell J A. et al. Crit. Care Med (2000) 28(10):3405-11) and in randomized controlled trials of new therapy in sepsis, acute respiratory distress syndrome (BERNARD G R. et al. N Engl J Med (1997) 336(13):912-8) and in critical care (HEBERT P C. et al. N Engl J Med (1999) 340(6) 409-17).

To further evaluate cardiovascular, respiratory, and renal function we also recorded, during each 24-hour period, vasopressor support, mechanical ventilation, and renal support, respectively. Vasopressor use was defined as dopamine >5 μg/kg/min or any dose of norepinephrine, epinephrine, vasopressin, or phenylephrine. Mechanical ventilation was defined as need for intubation and positive airway pressure (i.e. T-piece and mask ventilation were not considered ventilation). Renal support was defined as hemodialysis, peritoneal dialysis, or any continuous renal support mode (e.g. continuous veno-venous hemodialysis).

As a cumulative measure of the severity of SIRS, the presence of two, three or four of the SIRS criteria was scored each day over the 28-day observation period SIRS was considered present when subjects met at least two of four SIRS criteria. The SIRS criteria were 1) fever (>38° C.) or hypothermia (<36° C.), 2) tachycardia (>90 beats/min in the absence of beta-blockers, 3) tachypnea (>20 breaths/min) or need for mechanical ventilation, and 4) leukocytosis (total leukocyte count >12,000/μL or <4,000/μL).

TABLE 2C
Primary and secondary outcome variables for the ICU cohort and subsets
Survival and Days alive and free (DAF) of organ dysfunction Key

| | |
|---|---|
| SURVIVAL | 28-Day Survival |
| ALI.DAF | Days alive and free of acute Lung Injury |
| PRESS.DAF | Days alive and free of any vasopressors |
| PRESS2.DAF | Days alive and free of more than 2 ug/min of |
| | vasopressors |
| PRESS5.DAF | Days alive and free of more than 5 ug/min of |
| | vasopressors |
| PRESS15.DAF | Days alive and free of more than 15 ug/min of |
| | vasopressors |
| INO.DAF | Days alive and free of inotropes |
| SIRS2.DAF | Days alive and free of 2 of 4 SIRS criteria |
| SIRS3.DAF | Days alive and free of 3 of 4 SIRS crireria |
| SIRS4.DAF | Days alive and free of 4 of 4 SIRS criteria |
| STER.DAF | Days alive and free of steroids |
| CVS.DAF | Days alive and free of cardiovascular dysfunction |
| RESP.DAF | Days alive and free of respiratory dysfunction |
| PF300.DAF | Days alive and free of PaO2/FiO2 less than 300 mHg |
| VENT.DAF | Days alive and free of mechanical ventilators |
| CNS.DAF | Days alive and free of neurological dysfunction |
| COAG.DAF | Days alive and free of coagulation dysfunction |
| INR.DAF | Days alive and free of international normalized |
| | ratio >1.5 |
| ACRF.DAF | Days alive and free of acute renal failure |
| ANYREN.DAF | Days alive and free of any type of renal dysfunction |
| RENSUP.DAF | Days alive and free of renal support |
| ACHEP.DAF | Days alive and free of acute hepatic dysfunction |
| ANYHEP.DAF | Days alive and free of any type of hepatic dysfunction |
| AFFD.DAF | Days alive and free of acute Failure |
| FFD.DAF | Days alive and free of acute or chronic failure |

Baseline characteristics for the Biological Plausibility cohort included age in years. % males % smokers, % diabetes. % hypertension, ejection fraction, bypass time, clamp time and aprotinin. Outcome variables measured in the Biological Plausibility cohort included Granulocyte colony stimulating factor (GCSF). Interleukin 10 (IL10). Interleukin receptor 1a (IL1ra), Interleukin 6 (IL6), Interleukin 8 (IL8) and Monocyte Chemoattractant Protein 1 (MCP1). A key for the variables evaluated in the Biological Plausibility cohort is provided in TABLE 2D.

TABLE 2D
Biological plausibility key.
Biological Plausibility Key

| | |
|---|---|
| H.TENSE | Hypertensive (% hypertension) |
| EJEC.FRAC | Ejection Fraction |
| BYPASS | Bypass Time (hours) |
| CLAMP | Clamp Time (hours) |
| APROTININ | Aprotinin Use |
| GCSF | Granulocyte Colony Stimulating Factor (pg/mL) |
| IL10 | Interleukin 10 (pg/mL) |
| IL1ra | Interleukin receptor 1a (pg/mL) |
| IL6 | Interleukin 6 (pg/mL) |
| IL8 | Interleukin 8 (pg/mL) |
| MCP | Monocyte Chemoattractant Protein (pg/mL) |
| X.diff | DELTA for protein X preoperatively and 3 hours |
| | postoperatively |
| X.0 | protein X levels preoperatively |
| X.3 | protein X levels 3 hours postoperatively |

### Selection of SNPs for Genotyping

Publicly available genotype data was queried from the International HapMap Project (www.hapmap.org) and Perlegen Sciences. Inc. (www.perlegen.com) to select a set of tag SNPs (tSNPs) in the LNPEP, AVP and AVPR1A regions each having a minor allele frequency (MAF) greater than 0.05. These tSNPs were chosen using several statistical methods, including pairwise linkage disequilibrium (LD) measures (DEVLIN B. and RISCH N. Genomics (1995) 29:311-322), haplotype (STEPHENS M. et al. Am J Hum Genet. (2001) 68:978-989: and EXCOFFIER L. and SLATKIN M. Mol. Biol. Evol. (1995) 12(5):921-927) and haplotype block (HAWLEY M E. and KIDD K K. J. Heredity. (1995) 86:409-411) patterns, as well as phylogenetic (cladistic) distance metrics (HAWLEY M E. and KIDD K K. (1995)). When these methods did not yield a parsimonious conclusion, as was the case for AVP, SNPs closest in physical distance to the given gene of interest were selected. Each polymorphism was genotyped in the ICU Cohort and the Biological Plausibility Cohort.

### Sample Analysis

### Sample Preparation

Discarded whole blood samples, stored at 4° C., were collected from the hospital laboratory. DNA was extracted from buffy coat using the QIAamp DNA Midi kit (Qiagen. Mississauga, ON, Canada). After extraction, the DNA samples were transferred to 1.5 mL cryotubes, bar coded and cross-referenced with a unique patient number and stored at −80° C.

### ABI Genotyping

Single nucleotide polymorphisms in AVP. LNPEP and AVPR1A were genotyped using the 5′ nuclease. Taqman™ (Applied Biosystems; Foster City, Calif.) polymerase chain reaction (PCR) method. TABLE 2E provides a complete list of the 10 SNPs genotyped for this study.

TABLE 2E
List of tSNPs genotyped in ICU and Biological Plausibility Cohorts

| Gene | tSNPs | | | |
|---|---|---|---|---|
| LNPEP | rs10051637 | rs38041 | rs27711 | rs18059 |
| AVP | rs1410713 | rs857240 | rs857242 | |
| AVPR1A | rs3803107 | rs10877970 | rs1495027 | |

### Illumina Genotyping

Single nucleotide polymorphisms in AVP, LNPEP and AVPR1A were genotyped using the Illumina Golden Gate™ assay from 250 ng of DNA extracted from buffy coat. A list of these SNPs can be found labeled as cohort ‘I’ in TABLE 1B found in the General Methods section.

### Sequencing of LNPEP Region

Sequencing of a 157.1 kb region including the LNPEP and LRAP genes was undertaken using DNA extracted from six CEPH (i.e., Centre d'Etudes du Polymorphisme Humain) individuals obtained through the Coriell Institute for Medical Research using the Applied Biosystems 3730 platform. Ascertained polymorphisms were investigated for NCBI rs Id annotation using the UCSC genome browser (http://genome.ucsc.edu). If a polymorphism was found to not have an rs Id assigned, it was given a numeric id prefixed by ‘sirius’ (i.e. siriusx).

### Linkage Disequilibrium Analysis

Included in this patent are SNPs found to be associated with 28-day survival or response to vasopressin as well as SNPs determined to be in LD with the former. LD SNPs were ascertained using either Haploview (BARRETT J C. et al. Bioinformatics (2005) 21(2):263-5 (http://www.broad.mit.edu/mpg/haploview/)) or the LD function in the Genetics Package in R (R Core Development Group. 2005-R Development Core Team (www.R-project.org). A R² threshold of 0.5 was required in order that a SNP be considered in LD with those claimed herein. All LD SNPs are shown in table 1B.

The AVP, AVPR1A, LNPEP and LRAP genes are central to the action of vasopressin given that vasopressin induces vasoconstriction by signaling through the AVPR1A receptor and that vasopressin activity is inhibited when cleaved by LNPEP. Similar protein homology between LNPEP and LRAP suggest that these two genes arose through an ancient gene duplication event (DANCHIN E et al., Immunol Rev (2004) 198:216-332). This homology and the observation of an extended linkage disequilibrium (LD) block throughout the LRAP and LNPEP region (HapMap Phase II data; www.hapmap.org) supports the inclusion of LRAP in the vasopressin pathway.

Furthermore, variability in response to infused (i.e., administered) vasopressin most likely occurs as a result of polymorphisms in the AVP, AVPR1A. LNPEP and LRAP genes because the proteins that these genes encode are central to the actions of native and infused vasopressin (AVP).

### Statistical Analysis

A description of the statistical analysis used is provided for each example in the following sections.

### EXAMPLES

### Example 1

### Response to Vasopressin in Septic Shock

### Methods

### Cohort Selection

To investigate whether genotype predicts response to vasopressin, a subset of Caucasian subjects with septic shock and treated with vasopressin (N=103) were compared to a control group of Caucasian subjects with septic shock who had not been administered vasopressin (N=103). Vasopressin-treated and control subjects were matched based on age, gender, admission APACHE II score, medical versus surgical diagnosis and days alive and free of 3 of 4 systematic

inflammatory response syndrome (SIRS) criteria. The baseline characteristics of these groups are presented in Table 3.1.

TABLE 3.1
Baseline characteristics of cases (Caucasian ICU septic shock subjects treated with vasopressin)
and controls (Caucasian ICU subjects with septic shock, matched (see text for details) and not
treated with vasopressin). For age and APACHE II score, data is given as 25^{th} percentile|median|
75^{th} percentile. For all other variables, data is given as % (N/N total). N, number of subjects.

| | | Cases | | |
|---|---|---|---|---|
| | Control | (Vasopressin-treated) | Combined | Test |
| ALL | (N = 103) | (N = 103) | (N = 206) | Statistic |
| AGE | 44\|56\|71.5 | 47\|60\|68.5 | 44.25\|58.5\|70 | F = 0.14 d.f. = 1.204 P = 0.713 |
| GENDER | 69% (71/103) | 78% (80/103) | 73% (151/106) | X\hat{ }2 = 2.01 d.f. = 1 P = 0.156 |
| APACHE II | 24\|29\|34 | 25\|30\|37 | 24.25\|29\|34 | F = 0.38 d.f. = 1.204 P = 0.537 |
| % SURGICAL | 44% (45/103) | 44% (45/103) | 44% (90/206) | X\hat{ }2 = 0 d.f. = 1 P = 1 |

### Data Analysis

All data analysis was carried out using statistical packages available in R(R Core Development Group, 2005-R Development Core Team (www.R-project.org). R: A language and environment for statistical computing. Vienna, Austria. 2005). Chi-square and Kruskal-Wallis (KW) test statistics were used in conjunction with Cox proportional hazards (CPH) regression to identify significant SNP-phenotype associations, as well as to identify significantly different baseline characteristics (age, gender, admitting APACHE II score, and medical vs. surgical admitting diagnosis) requiring post-hoc, multivariate adjustment. The control population was selected by matching, using the MatchIt package in R, by age, gender, APACHE II score, medical vs. surgical diagnosis, and days alive and free of 3 of 4 SIRS criteria. There were no differences in baseline characteristics between vasopressin-treated cases and controls.

Using 28-day survival as the outcome variable and a chi-squared test of significance, SNP-phenotype comparisons were undertaken within and between treatment groups. We considered a by-genotype effect to be significant when two criteria were fulfilled. First, we expected an increase in 28-day survival for vasopressin-treated subjects compared to controls. Second, we required a p-value <0.1 for this difference in 28-day survival. When both criteria were met, we considered the allele or genotype predicting increased 28-day survival with vasopressin treatment to be an “improved response genotype” (IRG). Only IRG polymorphisms were evaluated for organ dysfunction results and were compared between vasopressin-treated subjects and matched controls using a Kruskal-Wallis test.

### Results

### 1.1 Leucyl/Cystinyl Aminopeptidase (LNPEP)

1.1.1 Adverse Response to Vasopressin Treatment of Subjects who have the CC Genotype of LNPEP rs18059 and Improved Response to Vasopressin Treatment of Subjects who have the TT Genotype of LNPEP rs18059

It was unknown whether SNPs within the LNPEP gene and those regions immediately upstream and downstream would be associated with the response to vasopressin. It was found that LNPEP rs18059 can be used to predict response (28-day survival) to vasopressin in subjects with septic shock. Of 103 vasopressin-treated and 103 matched-control subjects with septic shock, 73 and 81 were respectively genotyped for LNPEP rs18059. Baseline characteristics for subjects with genotypes are shown in Table 3.2 and Table 3.3.

TABLE 3.2
Baseline characteristics of a group of vasopressin-treated Caucasian septic-shock subjects by
genotype of leucyl/cystinyl aminopeptidase (LNPEP) rs18059.

| | CC | CT | TT | Combined | Test |
|---|---|---|---|---|---|
| VASOPRESSIN | (N = 27) | (N = 33) | (N = 13) | (N = 73) | Statistic |
| AGE | 44\|60\|69.5 | 48\|64\|72 | 39\|57\|66 | 47\|60\|68 | F = 0.7 d.f. = 2.70 P = 0.5 |
| GENDER | 67% (18/27) | 85% (28/33) | 77% (10/13) | 77% (56/73) | X\hat{ }2 = 2.75 d.f. = 2 P = 0.253 |
| APACHE II | 25\|32\|40 | 23\|30\|37 | 26\|29\|34 | 25\|30\|37 | F = 0.39 d.f. = 2.70 P = 0.678 |
| % SURGICAL | 48% (13/27) | 39% (13/33) | 31% (4/13) | 41% (30/73) | X\hat{ }2 = 1.17 d.f. = 2 P = 0.558 |

For age and APACHE II score, data is given as 25^{th} percentile|median|75^{th} percentile.
For all other variables, data is given as % (N/N total).
N, number of subjects.

TABLE 3.3
Baseline characteristics of a vasopressin untreated matched control group of Caucasian ICU septic
shock subjects by genotype of leucyl/cystinyl aminopeptidase (LNPEP) rs18059.

| | CC | CT | TT | Combined | Test |
|---|---|---|---|---|---|
| CONTROL | (N = 18) | (N = 43) | (N = 20) | (N = 81) | Statistic |
| AGE | 39.25\|46.5\|62.75 | 44\|52\|66.5 | 48.75\|67\|74 | 44\|56\|71.5 | F = 2.58 d.f. = 2.78 P = 0.0824 |
| GENDER | 83% (15/18) | 67% (29/43) | 50% (10/20) | 67% (54/81) | Chi = 4.76 d.f. = 2 P = 0.0925 |
| APACHE II | 23.25\|26.5\|32.5 | 26.5\|31\|37 | 25\|29\|34 | 24\|29\|34 | F = 2.24 d.f. = 2.78 P = 0.113 |
| % SURGICAL | 22% (4/18) | 33% (14/43) | 50% (10/20) | 35% (28/81) | Chi = 3.4 d.f. = 2 P = 0.183 |

For age and APACHE II score, data is given as 25^{th} percentile|median|75^{th} percentile.
For all other variables, data is given as % (N/N total).
N, number of subjects.

Table 3.4 and Table 3.5 show 28-day survival and organ dysfunction data by LNPEP rs18059 genotype for vasopressin-treated and control subjects respectively. Table 3.6 shows the differences in survival and measures of organ dysfunction between by LNPEP rs18059 genotype between vasopressin-treated and control subjects.

In general, Table 3.6 shows that vasopressin-treated subjects with LNPEP rs18059 CC had lower survival and more organ dysfunction than controls as evidenced by negative values for the LNPEP rs18059 CC subjects in the DELTA column. In contrast, vasopressin-treated subjects with the LNPEP rs18059 TT genotype had increased survival and improved organ function (shown by greater DAF) compared to controls as demonstrated by the generally positive values in DELTA, column. There was a small increase in survival of subjects with the LNPEP rs18059 CT genotype in vasopressin-treated subjects (36%) compared to controls (28%).

TABLE 3.4
A response association of leucyl/cystinyl aminopeptidase (LNPEP) rs18059 in a group of
Caucasian ICU septic shock subjects treated with vasopressin.

| VASOPRESSIN- | CC | CT | TT | Combined | Test |
|---|---|---|---|---|---|
| TREATED | (N = 27) | (N = 33) | (N = 13) | (N = 73) | Statistic |
| SURVIVAL | 44% (12/27) | 36% (12/33) | 38% (5/13) | 40% (29/73) | Chisquare = 0.42 d.f. = 2 P = 0.812 |
| DAYS ALIVE | 7.5\|19\|28 | 3\|13\|28 | 2\|8\|28 | 3\|13\|28 | F = 0.71 d.f. = 2.70 P = 0.496 |
| ALI.DAF | 2\|8\|16 | 1\|3\|19 | 1\|4\|12 | 1\|6\|17 | F = 0.23 d.f. = 2.70 P = 0.798 |
| PRESS.DAF | 0\|5\|19 | 0\|3\|18 | 0\|0\|22 | 0\|3\|19 | F = 0.21 d.f. = 2.70 P = 0.812 |
| PRESS2.DAF | 0\|5\|20.5 | 0\|3\|18 | 0\|0\|22 | 0\|3\|20 | F = 0.16 d.f. = 2.70 P = 0.855 |
| PRESS5.DAF | 0\|11\|20.5 | 0\|3\|19 | 0\|0\|23 | 0\|3\|21 | F = 0.12 d.f. = 2.70 P = 0.887 |
| PRESS15.DAF | 1\|12\|23 | 0\|6\|22 | 0\|0\|25 | 0\|7\|23 | F = 0.51 d.f. = 2.70 P = 0.6 |
| INO.DAF | 6\|12\|28 | 2\|12\|26 | 2\|8\|22 | 2\|12\|26 | F = 1.24 d.f. = 2.70 P = 0.296 |
| SIRS2.DAF | 0\|0\|3.5 | 0\|0\|2 | 0\|0\|1 | 0\|0\|2 | F = 0.12 d.f. = 2.70 P = 0.883 |
| SIRS3.DAF | 1.5\|4\|13.5 | 0\|4\|9 | 0\|2\|14 | 1\|4\|11 | F = 0.41 d.f. = 2.70 P = 0.667 |
| SIRS4.DAF | 5.5\|14\|21.5 | 2\|8\|23 | 2\|5\|20 | 2\|10\|23 | F = 0.51 d.f. = 2.70 P = 0.6 |
| STER.DAF | 0\|3\|17.5 | 1\|6\|20 | 1\|2\|7 | 1\|4\|19 | F = 0.19 d.f. = 2.70 P = 0.824 |
| CVS.DAF | 0\|2\|14.5 | 0\|0\|13 | 0\|0\|21 | 0\|1\|14 | F = 0.38 d.f. = 2.70 P = 0.684 |
| RESP.DAF | 0\|2\|7 | 0\|0\|5 | 0\|0\|8 | 0\|0\|8 | F = 0.56 d.f. = 2.70 P = 0.573 |
| PF300.DAF | 0\|0\|2 | 0\|0\|0 | 0\|0\|0 | 0\|0\|1 | F = 3.61 d.f. = 2.70 P = 0.0321 |
| VENT.DAF | 0\|0\|7 | 0\|0\|5 | 0\|0\|8 | 0\|0\|8 | F = 0.35 d.f. = 2.70 P = 0.707 |
| CNS.DAF | 6.5\|14\|27 | 2\|6\|24 | 2\|7\|24 | 2\|11\|25 | F = 1.29 d.f. = 2.70 P = 0.281 |
| COAG.DAF | 2\|11\|26.5 | 1\|5\|26 | 1\|7\|26 | 1\|8\|26 | F = 0.53 d.f. = 2.70 P = 0.588 |
| INR.DAF | 5.5\|15\|26.5 | 1\|8\|27 | 1\|5\|27 | 2\|8\|27 | F = 0.29 d.f. = 2.70 P = 0.746 |
| ACRF.DAF | 2.5\|8\|27 | 0\|2\|13 | 0\|2\|26 | 0\|5\|19 | F = 2.32 d.f. = 2.70 P = 0.106 |
| ANYREN.DAF | 2.5\|8\|24 | 0\|2\|13 | 0\|2\|26 | 0\|5\|18 | F = 1.8 d.f. = 2.70 P = 0.173 |
| RENSUP.DAF | 1\|6\|27.5 | 2\|5\|23 | 1\|3\|28 | 1\|5\|27 | F = 0.23 d.f. = 2.70 P = 0.796 |
| ACHEP.DAF | 1.5\|11\|24.5 | 2\|9\|24 | 2\|3\|28 | 2\|9\|27 | F = 0.1 d.f. = 2.70 P = 0.906 |
| ANYHEP.DAF | 1.5\|11\|24.5 | 2\|9\|24 | 2\|3\|28 | 2\|9\|27 | F = 0.07 d.f. = 2.70 P = 0.937 |

For 28-day survival, data is given as % (N survived/N total).
N, number of subjects.
For all variables besides 28-day survival, data is given as 25^{th} percentile|median|75^{th} percentile.

TABLE 3.5
A response association of leucyl/cystinyl aminopeptidase (LNPEP) rs18059 in a matched control
group of Caucasian ICU septic shock subjects not treated with vasopressin.

| | CC | CT | TT | Combined | Test |
|---|---|---|---|---|---|
| CONTROL | (N = 18) | (N = 43) | (N = 20) | (N = 81) | Statistic |
| SURVIVAL | 67% (12/18) | 28% (12/43) | 15% (3/20) | 33% | Chisquare = 12.59 d.f. = 2 P = 0.00184 |
| | | | | (27/81) | |
| DAYS ALIVE | 14.25\|28\|28 | 2\|6\|8 | 2.5\|5\|7.25 | 3\|8\|2 | F = 7.24 d.f. = 2.78 P = 0.00130 |
| ALI.DAF | 3.25\|12.5\|21.75 | 1\|2\|9 | 1\|3.5\|7 | 1\|5\|14 | F = 3.04 d.f. = 2.78 P = 0.0537 |
| PRESS.DAF | 9.25\|24.5\|26 | 0\|3\|17.5 | 0\|0\|4.25 | 0\|4\|22 | F = 7.98 d.f. = 2.78 P < 0.001 |
| PRESS2.DAF | 9.5\|24.5\|26 | 0\|3\|17.5 | 0\|0\|4.25 | 0\|4\|22 | F = 8.05 d.f. = 2.78 P < 0.001 |
| PRESS5.DAF | 10\|25.5\|27 | 0\|4\|19.5 | 0\|0.5\|5 | 0\|4\|23 | F = 7.69 d.f. = 2.78 P < 0.001 |
| PRESS15.DAF | 14.25\|26.5\|28 | 0\|5\|22 | 0\|2\|6.25 | 0\|5\|26 | F = 7.52 d.f. = 2.78 P = 0.00103 |
| INO.DAF | 14.25\|26.5\|28 | 2\|5\|20.5 | 0.75\|3\|7.25 | 2\|6\|28 | F = 5.54 d.f. = 2.78 P = 0.00561 |
| SIRS2.DAF | 0\|0.5\|10.75 | 0\|0\|1.5 | 0\|0\|0 | 0\|0\|1 | F = 2.28 d.f. = 2.78 P = 0.109 |
| SIRS3.DAF | 2\|4.5\|16.5 | 0\|2\|6 | 0.75\|1\|2 | 0\|2\|7 | F = 2.81 d.f. = 2.78 P = 0.0664 |
| SIRS4.DAF | 9.25\|16\|26.75 | 1\|5\|19.5 | 1.75\|3.5\|6.25 | 2\|6\|22 | F = 6.37 d.f. = 2.78 P = 0.00273 |
| STER.DAF | 2.75\|17\|27.5 | 1\|4\|1 | 1\|3.5\|7 | 1\|5\|21 | F = 1.78 d.f. = 2.78 P = 0.175 |
| CVS.DAF | 4.75\|21.5\|24.75 | 0\|2\|15.5 | 0\|0\|4 | 0\|2\|19 | F = 6.7 d.f. = 2.78 P = 0.00206 |
| RESP.DAF | 1.25\|8.5\|19.75 | 0\|1\|7.5 | 0\|0.5\|3.25 | 0\|1\|10 | F = 3.45 d.f. = 2.78 P = 0.0365 |
| PF300.DAF | 0\|0\|2 | 0\|0\|1 | 0\|0\|1 | 0\|0\|1 | F = 0.52 d.f. = 2.78 P = 0.598 |
| VENT.DAF | 0\|8.5\|19.75 | 0\|0\|7 | 0\|0\|1.5 | 0\|0\|10 | F = 3.53 d.f. = 2.78 P = 0.0342 |
| CNS.DAF | 11\|25.5\|27 | 0.5\|4\|23 | 0.75\|4\|7 | 1\|7\|25 | F = 8.55 d.f. = 2.78 P < 0.001 |
| COAG.DAF | 14.25\|28\|28 | 1\|3\|21 | 0.75\|5\|7.25 | 1\|6\|25 | F = 9 d.f. = 2.78 P < 0.001 |
| INR.DAF | 14\|24.5\|28 | 0\|3\|16.5 | 0\|3\|5.5 | 0\|4\|22 | F = 8.74 d.f. = 2.78 P < 0.001 |
| ACRF.DAF | 9.25\|22.5\|27 | 0\|4\|10.5 | 0\|0.5\|4 | 0\|4\|20 | F = 8.63 d.f. = 2.78 P < 0.001 |
| ANYREN.DAF | 9.25\|22.5\|27 | 0\|2\|10.5 | 0\|0\|4 | 0\|3\|20 | F = 9.64 d.f. = 2.78 P < 0.001 |
| RENSUP.DAF | 5.5\|23\|28 | 1\|2\|9.5 | 1\|2.5\|7.25 | 1\|4\|18 | F = 5.85 d.f. = 2.78 P = 0.00431 |
| ACHEP.DAF | 14.25\|28\|28 | 1\|4\|20 | 1\|5\|7.25 | 1\|6\|28 | F = 6.46 d.f. = 2.78 P = 0.00254 |
| ANYHEP.DAF | 14.25\|28\|28 | 1\|4\|20 | 1\|5\|7.25 | 1\|6\|28 | F = 6.73 d.f. = 2.78 P = 0.00201 |

For 28-day survival, data is given as % (N survived/N total).
N, number of subjects..
For all variables besides 28-day survival, data is given as 25^{th} percentile|median|75^{th} percentile.

TABLE 3.6
Difference in response association of leucyl/cystinyl aminopeptidase (LNPEP) rs18059 between
cases (vasopressin-treated group) (Treat) and controls (vasopressin untreated matched control)
(Cont) of Caucasian ICU subjects diagnosed with septic shock.

| | rs18059 CC | | | rs18059 CT | | | rs18059 TT | | |
|---|---|---|---|---|---|---|---|---|---|
| | (N = 27) | (N = 18) | | (N = 33) | (N = 43) | | (N = 13) | (N = 20) | |
| | Treat | Cont | DELTA | Treat | Cont | DELTA | Treat | Cont | DELTA |
| SURVIVAL | 44% (12) | 67% (12) | −23% | 36% (12) | 28% (12) | 8% | 38% (5) | 15% (3) | 23% |
| DAYS ALIVE | 19 | 28 | −9 | 13 | 6 | 7 | 8 | 5 | 3 |
| ALI.DAF | 8 | 12.5 | −4.5 | 3 | 2 | 1 | 4 | 3.5 | 0.5 |
| PRESS.DAF | 5 | 24.5 | −19.5 | 3 | 3 | 0 | 0 | 0 | 0 |
| PRESS2.DAF | 5 | 24.5 | −19.5 | 3 | 3 | 0 | 0 | 0 | 0 |
| PRESS5.DAF | 11 | 25.5 | −14.5 | 3 | 4 | −1 | 0 | 0.5 | −0.5 |
| PRESS15.DAF | 12 | 26.5 | −14.5 | 6 | 5 | 1 | 0 | 2 | −2 |
| INO.DAF | 12 | 26.5 | −14.5 | 12 | 5 | 7 | 8 | 3 | 5 |
| SIRS2.DAF | 0 | 0.5 | −0.5 | 0 | 0 | 0 | 0 | 0 | 0 |
| SIRS3.DAF | 4 | 4.5 | −0.5 | 4 | 2 | 2 | 2 | 1 | 1 |
| SIRS4.DAF | 14 | 16 | −2 | 8 | 5 | 3 | 5 | 3.5 | 1.5 |
| STER.DAF | 3 | 17 | −14 | 6 | 4 | 2 | 2 | 3.5 | −1.5 |
| CVS.DAF | 2 | 21.5 | −19.5 | 0 | 2 | −2 | 0 | 0 | 0 |
| RESP.DAF | 2 | 8.5 | −6.5 | 0 | 1 | −1 | 0 | 0.5 | −0.5 |
| PF300.DAF | 0 | 0 | 0 | 0 | 0 | 0 | 0 | 0 | 0 |
| VENT.DAF | 0 | 8.5 | −8.5 | 0 | 0 | 0 | 0 | 0 | 0 |
| CNS.DAF | 14 | 25.5 | −11.5 | 6 | 4 | 2 | 7 | 4 | 3 |
| COAG.DAF | 11 | 28 | −17 | 5 | 3 | 2 | 7 | 5 | 2 |
| INR.DAF | 15 | 24.5 | −9.5 | 8 | 3 | 5 | 5 | 3 | 2 |
| ACRF.DAF | 8 | 22.5 | −14.5 | 2 | 4 | −2 | 2 | 0.5 | 1.5 |
| ANYREN.DAF | 8 | 22.5 | −14.5 | 2 | 2 | 0 | 2 | 0 | 2 |
| RENSUP.DAF | 6 | 23 | −17 | 5 | 2 | 3 | 3 | 2.5 | 0.5 |
| ACHEP.DAF | 11 | 28 | −17 | 9 | 4 | 5 | 3 | 5 | −2 |
| ANYHEP.DAF | 11 | 28 | −17 | 9 | 4 | 5 | 3 | 5 | −2 |

For all variables besides 28-day survival, data is presented as medians.
For 28-day survival, data is presented as % (N survived/N total).
N, number of subjects.

A logistic regression approach was used to test for a statistically significant interaction between genotype and vasopressin use as predicted by 28-day survival TABLE 3.7 shows that there is a statistically significant interaction between LNPEP rs18059 genotype, vasopressin treatment and survival (P=0.0391), confirming that treatment with vasopressin decreases 28-day survival in LNPEP rs18059 CC subjects. In contrast, 28-day survival for vasopressin-treated subjects with the LNPEP rs18059 TT genotype is improved compared with controls. Following adjustment for age, admission APACHE II score, sender, medical, surgical diagnosis and 3 of 4 systematic inflammatory response syndrome (SIRS) criteria, there was still a statistically significant interaction of the LNPEP rs18059 genotype, treatment with vasopressin and survival (P=0.0555)

TABLE 3.7
Interaction between vasopressin use vs. no vasopressin use (controls)
and CC or CT genotype vs. TT genotype of leucyl/cystinyl
aminopeptidase (LNPEP) rs18059 on 28-day survival.

| | Estimate | Std. Error | z value | Pr(>\|z\|) |
|---|---|---|---|---|
| Vasopressin vs. controls + | −2.1809 | 1.057 | −2.063 | 0.03908 |
| genotype interaction | | | | |
| Vasopressin vs. controls + | −2.2301 | 1.165 | −1.914 | 0.05559 |
| genotype interaction − | | | | |
| Adjusted | | | | |

1.1.2 Adverse Response to Vasopressin Treatment of Subjects who have the AA Genotype of LNPEP rs27711 and Improved Response to Vasopressin Treatment of Subjects who have the GG Genotype of LNPEP rs27711

It was unknown whether SNPs within the LNPEP gene and those regions immediately upstream and downstream are associated with the response to vasopressin. It was found that LNPEP rs27711 can be used to predict response to vasopressin in subjects with septic shock using 28-day survival and measures of organ dysfunction as outcome variables. Of 103 vasopressin-treated and 103 matched-control subjects with septic shock. 70 and 81 were respectively genotyped for LNPEP rs27711. Baseline characteristics for subjects with genotypes are shown in Table 3.8 and Table 3.9. LNPEP rs27711 is in linkage disequilibrium with, for example, LNPEP rs18059 and LNPEP rs10051637, which were also genotyped in this cohort.

TABLE 3.8
Baseline characteristics of vasopressin-treated Caucasian septic-shock subjects by LNPEP rs27711
genotype.

| | AA | AG | GG | Combined | Test |
|---|---|---|---|---|---|
| VASOPRESSIN | (N = 21) | (N = 28) | (N = 21) | (N = 70) | Statistic |
| AGE | 43\|58\|71 | 50.25\|63.5\|72 | 39\|60\|68 | 47\|60\|68.5 | F = 0.32 d.f. = 2.67 P = 0.728 |
| GENDER | 71% (15/21) | 75% (21/28) | 81% (17/21) | 76% (53/70) | X\hat{ }2 = 0.53 d.f. = 2 P = 0.767 |
| APACHE II | 25\|33\|41 | 23.75\|29.5\|36.25 | 26\|29\|36 | 25\|30\|37 | F = 0.68 d.f. = 2.67 P = 0.512 |
| % SURGICAL | 43% (9/21) | 46% (13/28) | 29% (6/21) | 40% (28/70) | X\hat{ }2 = 1.7 d.f. = 2 P = 0.428 |

For age and APACHE II score, data is given as 25^{th} percentile|median|75^{th} percentile.
For all other variables, data is given as % (N/N total).
N, number of subjects.

TABLE 3.9
Baseline characteristics of a group of Caucasian septic-shock control subjects by LNPEP rs27711
genotype.

| | AA | AG | GG | Combined | Test |
|---|---|---|---|---|---|
| CONTROL | (N = 10) | (N = 45) | (N = 26) | (N = 81) | Statistic |
| AGE | 39.25\|45.5\|58.5 | 43\|52\|67 | 49\|66\|74 | 44\|56\|71.5 | F = 3.59 d.f. = 2.78 P = 0.0322 |
| GENDER | 80% (8/10) | 67% (30/45) | 62% (16/26) | 67% (54/81) | X\hat{ }2 = 1.11 d.f. = 2 P = 0.575 |
| APACHE II | 23.25\|26\|32.5 | 26\|30\|34 | 27\|30.5\|38 | 24\|29\|34 | F = 1.26 d.f. = 2.78 P = 0.29 |
| % SURGICAL | 20% (2/10) | 36% (16/45) | 38% (10/26) | 35% (28/81) | X\hat{ }2 = 1.13 d.f. = 2 P = 0.568 |

For age and APACHE II score, data is given as 25^{th} percentile|median|75^{th} percentile.
For all other variables, data is given as % (N/N total).
N, number of subjects.

Tables 3.10, 3.11 and 3.12 contain 28-day survival and organ dysfunction data for septic-shock subjects genotyped for LNPEP rs27711. In general, vasopressin-treated subjects with the LNPEP rs27711 AA genotype had a dramatically decreased survival (43%) compared to controls (60%) as demonstrated by the negative values in the LNPEP rs27711 AA DELTA column in Table 3.12. In general, vasopressin-treated subjects with the LNPEP rs27711 AA genotype also had increased organ dysfunction as demonstrated by fewer DAF of organ dysfunction compared with controls. In contrast, vasopressin-treated subjects with the LNPEP rs27711 GG genotype had an increased survival (33%) compared to controls (19%) as demonstrated by the positive values in the LNPEP rs27711 GG DELTA column in Table 3.12.

TABLE 3.10
A response association of leucyl/cystinyl aminopeptidase (LNPEP) rs27711 in a group of
Caucasian ICU septic shock subjects who were treated with vasopressin. For all variables besides
28-day survival, data is given as 25^{th} percentile|median|75^{th} percentile. For 28-day survival, data
is given as % (N survived/N total).

| | AA | AG | GG | Combined | Test |
|---|---|---|---|---|---|
| VASOPRESSIN | (N = 21) | (N = 28) | (N = 21) | (N = 70) | Statistic |
| SURVIVAL | 43% (9) | 36% (10) | 33% (7) | 37% (26) | Chisquare = 0.45 d.f. = 2 P = 0.799 |
| DAYS ALIVE | 7\|12\|28 | 3\|17.5\|28 | 2\|8\|28 | 3\|12.5\|28 | F = 0.49 d.f. = 2.67 P = 0.615 |
| ALI.DAF | 2\|6\|12 | 2\|9\|21 | 1\|2\|12 | 1\|5.5\|17 | F = 1.65 d.f. = 2.67 P = 0.201 |
| PRESS.DAF | 0\|1\|19 | 0\|4\|16.25 | 0\|0\|21 | 0\|1\|18 | F = 0.03 d.f. = 2.67 P = 0.97 |
| PRESS2.DAF | 0\|1\|20 | 0\|4\|16.25 | 0\|0\|21 | 0\|1\|18 | F = 0.04 d.f. = 2.67 P = 0.96 |
| PRESS5.DAF | 0\|2\|20 | 0\|7.5\|18 | 0\|0\|21 | 0\|1.5\|19.75 | F = 0.09 d.f. = 2.67 P = 0.91 |
| PRESS15.DAF | 1\|7\|23 | 0\|11.5\|21.25 | 0\|2\|21 | 0\|5\|22 | F = 0.4 d.f. = 2.67 P = 0.672 |
| INO.DAF | 7\|12\|28 | 2\|14\|26 | 2\|5\|22 | 2\|12\|26 | F = 0.99 d.f. = 2.67 P = 0.375 |
| SIRS2.DAF | 0\|0\|3 | 0\|1\|2 | 0\|0\|1 | 0\|0\|2.75 | F = 0.24 d.f. = 2.67 P = 0.787 |
| SIRS3.DAF | 1\|4\|7 | 1\|7\|12.5 | 0\|2\|8 | 1\|4\|11 | F = 1.13 d.f. = 2.67 P = 0.33 |
| SIRS4.DAF | 5\|10\|19 | 2\|15\|24 | 2\|5\|20 | 2\|10\|21.5 | F = 0.5 d.f. = 2.67 P = 0.61 |
| STER.DAF | 0\|2\|12 | 1\|10\|24.25 | 1\|3\|10 | 1\|4\|18.25 | F = 0.98 d.f. = 2.67 P = 0.382 |
| CVS.DAF | 0\|1\|14 | 0\|0.5\|13 | 0\|0\|14 | 0\|0\|13.75 | F = 0.1 d.f. = 2.67 P = 0.903 |
| RESP.DAF | 0\|1\|4 | 0\|0\|5 | 0\|0\|8 | 0\|0\|5 | F = 0.21 d.f. = 2.67 P = 0.812 |
| PF300.DAF | 0\|0\|2 | 0\|0\|1.25 | 0\|0\|0 | 0\|0\|1 | F = 3 d.f. = 2.67 P = 0.0565 |
| VENT.DAF | 0\|0\|3 | 0\|0\|2.75 | 0\|0\|8 | 0\|0\|4.5 | F = 0.01 d.f. = 2.67 P = 0.991 |
| CNS.DAF | 6\|11\|27 | 2\|13\|24 | 2\|7\|24 | 2\|11\|24 | F = 0.67 d.f. = 2.67 P = 0.513 |
| COAG.DAF | 2\|8\|25 | 1\|13.5\|27.25 | 1\|6\|26 | 1\|8\|26 | F = 0.18 d.f. = 2.67 P = 0.84 |
| INR.DAF | 4\|11\|26 | 1.75\|11.5\|27 | 1\|5\|26 | 2\|8\|26.75 | F = 0.29 d.f. = 2.67 P = 0.747 |
| ACRF.DAF | 2\|6\|24 | 0\|2\|18.25 | 0\|4\|14 | 0\|5\|19 | F = 0.5 d.f. = 2.67 P = 0.607 |
| ANYREN.DAF | 2\|6\|24 | 0\|2\|16.5 | 0\|4\|14 | 0\|5\|17.5 | F = 0.47 d.f. = 2.67 P = 0.629 |
| RENSUP.DAF | 1\|3\|27 | 2\|7.5\|23.5 | 2\|5\|23 | 1\|5.5\|24.5 | F = 0.5 d.f. = 2.67 P = 0.607 |
| ACHEP.DAF | 1\|7\|24 | 3\|14\|24.75 | 2\|4\|28 | 2\|9\|24.75 | F = 0.78 d.f. = 2.67 P = 0.462 |
| ANYHEP.DAF | 1\|7\|24 | 3\|14\|24.75 | 2\|4\|28 | 2\|9\|24.75 | F = 0.77 d.f. = 2.67 P = 0.466 |

N, number of subjects.

TABLE 3.11
A response association of leucyl/cystinyl aminopeptidase (LNPEP) rs27711 in a matched control
group of Caucasian ICU septic shock subjects who were treated with vasopressin. For all variables
besides 28-day survival, data is given as 25^{th} percentile|median|75^{th} percentile.
For 28-day survival, data is given as % (N survived/N total).

| | AA | AG | GG | Combined | Test |
|---|---|---|---|---|---|
| CONTROL | (N = 10) | (N = 45) | (N = 26) | (N = 81) | Statistic |
| SURVIVAL | 60% (6) | 36% (16) | 19% (5) | 33% (27) | Chisquare = 5.63 d.f. = 2 P = 0.06 |
| DAYS ALIVE | 14.25\|28\|28 | 2\|8\|28 | 3\|5.5\|8.75 | 3\|8\|28 | F = 5.09 d.f. = 2.78 P = 0.00839 |
| ALI.DAF | 7\|9.5\|19.25 | 1\|2\|18 | 1\|5\|8 | 1\|5\|15 | F = 2.04 d.f. = 2.78 P = 0.136 |
| PRESS.DAF | 10.75\|23\|26.75 | 0\|4\|22 | 0\|1.5\|5.75 | 0\|4\|22 | F = 4.35 d.f. = 2.78 P = 0.0161 |
| PRESS2.DAF | 11.5\|23\|26.75 | 0\|4\|2 | 0\|1.5\|5.75 | 0\|4\|22 | F = 4.41 d.f. = 2.78 P = 0.0154 |
| PRESS5.DAF | 13\|25\|27 | 0\|4\|23 | 0\|1.5\|6.5 | 0\|4\|23 | F = 0.67 d.f. = 2.78 P = 0.0122 |
| PRESS15.DAF | 14.25\|26.5\|28 | 1\|6\|25 | 0\|2.5\|7 | 1\|6\|26 | F = 5.11 d.f. = 2.78 P = 0.00823 |
| INO.DAF | 14.25\|28\|28 | 2\|6\|25 | 1\|3.5\|8 | 2\|6\|28 | F = 3.76 d.f. = 2.78 P = 0.0276 |
| SIRS2.DAF | 0\|1\|4 | 0\|0\|2 | 0\|0\|1 | 0\|0\|1 | F = 1.59 d.f. = 2.78 P = 0.211 |
| SIRS3.DAF | 2\|3.5\|6.5 | 0\|2\|9 | 0.25\|1\|2 | 0\|2\|7 | F = 1.19 d.f. = 2.78 P = 0.308 |
| SIRS4.DAF | 9.25\|10.5\|23 | 1\|7\|22 | 2\|4\|7 | 2\|7\|22 | F = 3.72 d.f. = 2.78 P = 0.0286 |
| STER.DAF | 8.5\|17\|26.25 | 1\|4\|24 | 1\|4.5\|7.75 | 1\|5\|21 | F = 1.37 d.f. = 2.78 P = 0.26 |
| CVS.DAF | 7.5\|21.5\|23.75 | 0\|2\|18 | 0\|0\|4 | 0\|3\|19 | F = 4.48 d.f. = 2.78 P = 0.0144 |
| RESP.DAF | 4.75\|11\|20.75 | 0\|1\|9 | 0\|1\|3.75 | 0\|1\|10 | F = 3.5 d.f. = 2.78 P = 0.035 |
| PF300.DAF | 0\|1.5\|2 | 0\|0\|1 | 0\|0\|1 | 0\|0\|1 | F = 2.04 d.f. = 2.78 P = 0.137 |
| VENT.DAF | 4\|10\|20 | 0\|0\|9 | 0\|0\|2.75 | 0\|0\|10 | F = 3.16 d.f. = 2.78 P = 0.048 |
| CNS.DAF | 11\|24.5\|26 | 1\|7\|25 | 0\|4\|8.5 | 1\|7\|25 | F = 4.78 d.f. = 2.78 P = 0.011 |
| COAG.DAF | 14.25\|28\|28 | 1\|4\|24 | 1\|5\|8 | 1\|6\|25 | F = 6.32 d.f. = 2.78 P = 0.00287 |
| INR.DAF | 14\|26.5\|28 | 1\|4\|22 | 0\|3\|6.5 | 0\|5\|22 | F = 7.51 d.f. = 2.78 P = 0.00104 |
| ACRF.DAF | 11\|20\|27.75 | 1\|5\|20 | 0\|0.5\|4.75 | 0\|4\|20 | F = 8.6 d.f. = 2.78 P < 0.001 |
| ANYREN.DAF | 11\|20\|27.75 | 0\|3\|20 | 0\|0\|4.75 | 0\|4\|20 | F = 8.38 d.f. = 2.78 P < 0.001 |
| RENSUP.DAF | 11\|21.5\|28 | 1\|3\|18 | 1\|3\|8 | 1\|4\|18 | F = 3.51 d.f. = 2.78 P < 0.0.346 |
| ACHEP.DAF | 14.25\|28\|28 | 1\|6\|22 | 1.25\|5\|7.75 | 1\|6\|28 | F = 3.65 d.f. = 2.78 P = 0.0304 |
| ANYHEP.DAF | 14.25\|28\|28 | 1\|5\|22 | 1.25\|5\|7.75 | 1\|6\|28 | F = 3.64 d.f. = 2.78 P = 0.0309 |

N, number of subjects.

TABLE 3.12
Difference in response association of leucyl/cystinyl aminopeptidase (LNPEP) rs27711 between
cases (vasopressin-treated group) (Treat) and controls (vasopressin untreated matched control)
(Cont) of Caucasian ICU subjects diagnosed with septic shock. For all variables besides 28-day
survival, data is presented as medians. For 28-day survival, data is presented as %(N survived/N
total).

| | AA | AA | | AG | AG | | GG | GG | |
|---|---|---|---|---|---|---|---|---|---|
| | (N = 21) | (N = 10) | | (N = 28) | (N = 45) | | (N = 21) | (N = 26) | |
| | Treat | Cont | DELTA | Treat | Cont | DELTA | Treat | Cont | DELTA |
| SURVIVAL | 43% (9) | 60%(6) | −18% | 36% (10) | 36% (16) | 0% | 33% (7) | 19% (5) | 14% |
| DAYS ALIVE | 12 | 28 | −16 | 17.5 | 8 | 9.5 | 8 | 5.5 | 2.5 |
| ALI.DAF | 6 | 9.5 | −3.5 | 9 | 2 | 7 | 2 | 5 | −3 |
| PRESS.DAF | 1 | 23 | −22 | 4 | 4 | 0 | 0 | 1.5 | −1.5 |
| PRESS2.DAF | 1 | 23 | −22 | 4 | 4 | 0 | 0 | 1.5 | −1.5 |
| PRESS5.DAF | 2 | 25 | −23 | 7.5 | 4 | 3.5 | 0 | 1.5 | −1.5 |
| PRESS15.DAF | 7 | 26.5 | −19.5 | 11.5 | 6 | 5.5 | 2 | 2.5 | −0.5 |
| INO.DAF | 12 | 28 | −16 | 14 | 6 | 8 | 5 | 3.5 | 1.5 |
| SIRS2.DAF | 0 | 1 | −1 | 1 | 0 | 1 | 0 | 0 | 0 |
| SIRS3.DAF | 4 | 3.5 | 0.5 | 7 | 2 | 5 | 2 | 1 | 1 |
| SIRS4.DAF | 10 | 10.5 | −0.5 | 15 | 7 | 8 | 5 | 4 | 1 |
| STER.DAF | 2 | 17 | −15 | 10 | 4 | 6 | 3 | 4.5 | −1.5 |
| CVS.DAF | 1 | 21.5 | −20.5 | 0.5 | 2 | −1.5 | 0 | 0 | 0 |
| RESP.DAF | 1 | 11 | −10 | 0 | 1 | −1 | 0 | 1 | −1 |
| PF300.DAF | 0 | 1.5 | −1.5 | 0 | 0 | 0 | 0 | 0 | 0 |
| VENT.DAF | 0 | 10 | −10 | 0 | 0 | 0 | 0 | 0 | 0 |
| CNS.DAF | 11 | 24.5 | −13.5 | 13 | 7 | 6 | 7 | 4 | 3 |
| COAG.DAF | 8 | 28 | −20 | 13.5 | 4 | 9.5 | 6 | 5 | 1 |
| INR.DAF | 11 | 26.5 | −15.5 | 11.5 | 4 | 7.5 | 5 | 3 | 2 |
| ACRF.DAF | 6 | 20 | −14 | 2 | 5 | −3 | 4 | 0.5 | 3.5 |
| ANYREN.DAF | 6 | 20 | −14 | 2 | 3 | −1 | 4 | 0 | 4 |
| RENSUP.DAF | 3 | 21.5 | −18.5 | 7.5 | 3 | 4.5 | 5 | 3 | 2 |
| ACHEP.DAF | 7 | 28 | −21 | 14 | 6 | 8 | 4 | 5 | −1 |
| ANYHEP.DAF | 7 | 28 | −21 | 14 | 5 | 9 | 4 | 5 | −1 |

N, number of subjects.

1.1.3 Adverse Response to Vasopressin Treatment of Subjects who have the GG Genotype of LNPEP rs10051637

It was unknown whether SNPs within the LNPEP gene and those regions immediately upstream and downstream are associated with the response to vasopressin. It was found that LNPEP rs10051637 can be used to predict response to vasopressin in subjects with septic shock using 28-day survival and measures of organ dysfunction as outcome variables. Of 103 vasopressin-treated and 103 matched-control subjects with septic shock, 72 and 81 were respectively genotyped for LNPEP rs10051637. Baseline characteristics for subjects with genotypes are shown in Table 3.13 and Table 3.14. LNPEP rs10051637 is in linkage disequilibrium with, for example LNPEP rs18059 and LNPEP G9419812A, which were also genotyped in this cohort.

TABLE 3.13
Baseline characteristics of a group of vasopressin-treated Caucasian septic shock subjects
leucyl/cystinyl aminopeptidase (LNPEP) rs10051637 genotype. For age and APACHE II score,
data is given as 25^{th} percentile|median|75^{th} percentile. For all other variables, data is given as %
(N/N total).

| | AA | AG | GG | Combined | Test |
|---|---|---|---|---|---|
| VASOPRESSIN | (N = 19) | (N = 29) | (N = 24) | (N = 72) | Statistic |
| AGE | 38\|60\|68 | 54\|65 72 | 42.75\|55\|68.75 | 47\|60\|68.5 | F = 0.89 d.f. = 2.69 P = 0.417 |
| GENDER | 79% (15/19) | 79% (23/29) | 71% (17/24) | 76% (55/72) | X\hat{ }2 = 0.62 d.f. = 2 P = 0.735 |
| APACHE II | 25.5\|28\|35 | 23\|30\|37 | 25 32.5\|40.25 | 25\|30\|37 | F = 0.49 d.f. = 2.69 P = 0.616 |
| % SURGICAL | 26% (15/19) | 48% (14/29) | 38% (9/24) | 39% (28/72) | X\hat{ }2 = 2.36 d.f. = 2 P = 0.308 |

N, number of subjects.

TABLE 3.14
Baseline characteristics of a matched-control group of Caucasian septic-shock subjects by
leucyl/cystinyl aminopeptidase (LNPEP) rs10051637 genotype. For age and APACHE II score,
data is given as 25^{th} percentile|median|75^{th} percentile. For all other variables, data is given as %
(N/N total).

| | AA | AG | GG | Combined | Test |
|---|---|---|---|---|---|
| CONTROL | (N = 25) | (N = 46) | (N = 10) | (N = 81) | Statistic |
| AGE | 49\|67\|74 | 43.25\|52\|66.5 | 39.25\|45.5\|58.5 | 44\|56\|71.5 | F = 3.91 d.f. = 2.78 P = 0.024 |
| GENDER | 60% (15/25) | 67% (31/46) | 80% (8/10) | 67% (54/81) | X\hat{ }2 = 1.31 d.f. = 2 P = 0.519 |
| APACHE II | 27\|29\|38 | 26\|30\|34 | 23.25\|26\|32.5 | 24\|29\|34 | F = 1.04 d.f. = 2.78 P = 0.359 |
| % SURGICAL | 40% (10/25) | 35% (16/46) | 20% (2/10) | 35% (28/81) | X\hat{ }2 = 1.27 d.f. = 2 P = 0.531 |

N, number of subjects.

Tables 3.15, 3.16 and Tables 3.17 contain 28-day survival and organ dysfunction data for septic-shock subjects genotyped for LNPEP rs10051637. Vasopressin-treated subjects with the LNPEP rs10051637 GG genotype had a dramatically decreased survival (46%) compared to controls (60%) as demonstrated by the negative values in the LNPEP rs10051637 GG DELTA column in Table 3.17. Vasopressin-treated subjects with the LNPEP rs10051637 GG genotype were also observed to have more organ dysfunction as demonstrated by fewer DAF of organ dysfunction. In contrast, vasopressin-treated subjects with the LNPEP rs10051637 AG and AA genotypes had increased survival (26%) compared to controls (20%).

TABLE 3.15
A response association of leucyl/cystinyl aminopeptidase (LNPEP) rs10051637 and use of
vasopressin in a group of vasopressin-treated Caucasian ICU septic-shock subjects. For all
variables besides 28-day survival, data is given as 25^{th} percentile|median|75^{th} percentile.
For 28-day survival, data is given as % (N survived/N total).

| | AA | AG | GG | Combined | Test |
|---|---|---|---|---|---|
| VASOPRESSIN | (N = 19) | (N = 29) | (N = 24) | (N = 72) | Statistic |
| SURVIVAL | 26% (5/19) | 38% (11/29) | 46% (11/24) | 38% (27/72) | Chisquare = 1.73 d.f. = 2 P = 0.422 |
| DAYS ALIVE | 2\|6\|25.5 | 3\|20\|28 | 7\|15.5\|28 | 3\|12.5\|28 | F = 1.08 d.f. = 2.69 P = 0.345 |
| ALI.DAF | 1\|2\|6 | 2\|10\|24 | 1.75\|6.5\|13 | 1\|5.5\|17 | F = 2.68 d.f. = 2.69 P = 0.0754 |
| PRESS.DAF | 0\|0\|17.5 | 0\|5\|17 | 0\|6.5\|19.5 | 0\|1\|18 | F = 0.43 d.f. = 2.69 P = 0.651 |
| PRESS2.DAF | 0\|0\|19 | 0\|5\|17 | 0\|6.5\|21 | 0\|1\|18 | F = 0.44 d.f. = 2.69 P = 0.646 |
| PRESS5.DAF | 0\|0\|19.5 | 0\|8\|18 | 0\|8\|21.25 | 0\|1.5\|20 | F = 0.48 d.f. = 2.69 P = 0.619 |
| PRESS15.DAF | 0\|1\|20.5 | 0\|12\|21 | 0.75\|12\|23.25 | 0\|5\|22.25 | F = 1.02 d.f. = 2.69 P = 0.364 |
| INO.DAF | 1.5\|4\|17.5 | 2\|15\|26 | 6.5\|12\|28 | 2\|12\|26 | F = 2.31 d.f. = 2.69 P = 0.107 |
| SIRS2.DAF | 0\|0\|1 | 0\|1\|2 | 0\|0\|3.25 | 0\|0\|2 | F = 0.51 d.f. = 2.69 P = 0.605 |
| SIRS3.DAF | 0\|1\|6 | 1\|7\|11 | 1\|3.5\|8.5 | 0.75\|3.5\|11 | F = 1.54 d.f. = 2.69 P = 0.221 |
| SIRS4.DAF | 1.5\|4\|18 | 2\|16\|24 | 4.5\|10.5\|20 | 2\|10\|22.25 | F = 1 d.f. = 2.69 P = 0.372 |
| STER.DAF | 1\|2\|6 | 1\|9\|16 | 0\|2.5\|20.25 | 1\|3.5\|16 | F = 0.8 d.f. = 2.69 P = 0.455 |
| CVS.DAF | 0\|0\|9 | 0\|1\|13 | 0\|1.5\|16.25 | 0\|0\|14 | F = 0.58 d.f. = 2.69 P = 0.56 |
| RESP.DAF | 0\|0\|1 | 0\|0\|5 | 0\|1\|7.5 | 0\|0\|5.25 | F = 0.93 d.f. = 2.69 P = 0.401 |
| PF300.DAF | 0\|0\|0 | 0\|0\|1 | 0\|0\|2 | 0\|0\|1 | F = 5.18 d.f. = 2.69 P = 0.0079 |
| VENT.DAF | 0\|0\|0 | 0\|0\|5 | 0\|0\|7.5 | 0\|0\|5.25 | F = 0.36 d.f. = 2.69 P = 0.697 |
| CNS.DAF | 2\|5\|19 | 2\|13\|24 | 6\|11.5\|27.25 | 2\|11\|24.25 | F = 1.35 d.f. = 2.69 P = 0.265 |
| COAG.DAF | 1\|5\|16.5 | 1\|12\|26 | 1.75\|9\|25.75 | 1\|7.5\|26 | F = 0.41 d.f. = 2.69 P = 0.666 |
| INR.DAF | 1\|5\|23.5 | 2\|13\|27 | 3.5\|13\|27 | 1.75\|8\|27 | F = 0.81 d.f. = 2.69 P = 0.448 |
| ACRF.DAF | 0\|3\|12 | 0\|2\|16 | 1.75\|6\|27 | 0\|4.5\|19 | F = 1.21 d.f. = 2.69 P = 0.303 |
| ANYREN.DAF | 0\|3\|12 | 0\|2\|13 | 1.75\|6\|24.75 | 0\|4.5\|16.5 | F = 1.16 d.f. = 2.69 P = 0.318 |
| RENSUP.DAF | 2\|4\|16.5 | 2\|6\|20 | 0.75\|4.5\|28 | 1\|4.5\|23.5 | F = 0.1 d.f. = 2.69 P = 0.908 |
| ACHEP.DAF | 2\|3\|21 | 3\|15\|27 | 1\|8.5\|24.25 | 2\|9\|25.5 | F = 1.19 d.f. = 2.69 P = 0.309 |
| ANYHEP.DAF | 2\|3\|21 | 3\|15\|27 | 1\|8.5\|24.25 | 2\|9\|25.5 | F = 1.25 d.f. = 2.69 P = 0.293 |

N, number of subjects.

TABLE 3.16
A response association of leucyl/cystinyl aminopeptidase (LNPEP) rs10051637 and use of
vasopressin in a matched control group of Caucasian ICU septic shock subjects who were not
treated with vasopressin. For all variables besides 28-day survival, data is given as 25^{th} percentile|
median|75^{th} percentile. For 28-day survival, data is given as % (N survived/N total).

| | AA | AG | GG | Combined | Test |
|---|---|---|---|---|---|
| CONTROL | (N = 25) | (N = 46) | (N = 10) | (N = 81) | Statistic |
| SURVIVAL | 20% (5/25) | 35% (16/46) | 60% (6/10) | 33% (27/81) | Chisquare = 5.24 d.f. = 2 P = 0.0727 |
| DAYS ALIVE | 3\|5\|8 | 2\|8\|28 | 14.25\|28\|28 | 3\|8\|28 | F = 5.18 d.f. = 2.78 P = 0.0077 |
| ALI.DAF | 1\|5\|8 | 1\|2.5\|17.25 | 7\|9.5\|19.25 | 1\|5\|15 | F = 2.04 d.f. = 2.78 P = 0.137 |
| PRESS.DAF | 0\|2\|6 | 0\|3.5\|21.25 | 10.75\|23\|26.75 | 0\|4\|22 | F = 4.27 d.f. = 2.78 P = 0.0174 |
| PRESS2.DAF | 0\|2\|6 | 0\|3.5\|21.25 | 11.5\|23\|26.75 | 0\|4\|22 | F = 4.32 d.f. = 2.78 P = 0.0166 |
| PRESS5.DAF | 0\|2\|7 | 0.25\|4\|22.5 | 13\|25\|27 | 0\|4\|23 | F = 4.52 d.f. = 2.78 P = 0.0138 |
| PRESS15.DAF | 0\|3\|7 | 1\|5.5\|25 | 14.25\|26.5\|28 | 1\|6\|26 | F = 4.9 d.f. = 2.78 P = 0.0099 |
| INO.DAF | 1\|3\|8 | 2\|6\|24.5 | 14.25\|28\|28 | 2\|6\|28 | F = 3.9 d.f. = 2.78 P = 0.0243 |
| SIRS2.DAF | 0\|0\|1 | 0\|0\|1.75 | 0\|1\|4 | 0\|0\|1 | F = 1.57 d.f. = 2.78 P = 0.214 |
| SIRS3.DAF | 1\|1\|2 | 0\|2\|9 | 2\|3.5\|6.5 | 0\|2\|7 | F = 0.94 d.f. = 2.78 P = 0.395 |
| SIRS4.DAF | 2\|4\|7 | 1.25\|6.5\|22 | 9.25\|10.5\|23 | 2\|7\|22 | F = 3.59 d.f. = 2.78 P = 0.0322 |
| STER.DAF | 1\|5\|8 | 1\|4\|21.75 | 8.5\|17\|26.25 | 1\|5\|21 | F = 1.37 d.f. = 2.78 P = 0.261 |
| CVS.DAF | 0\|0\|4 | 0\|2\|18 | 7.5\|21.5\|23.75 | 0\|3\|19 | F = 4.27 d.f. = 2.78 P = 0.0174 |
| RESP.DAF | 0\|1\|4 | 0\|1\|9 | 4.75\|11\|20.75 | 0\|1\|10 | F = 3.46 d.f. = 2.78 P = 0.0364 |
| PF300.DAF | 0\|0\|1 | 0\|0\|0.75 | 0\|1.5\|2 | 0\|0\|1 | F = 2.26 d.f. = 2.78 P = 0.111 |
| VENT.DAF | 0\|0\|3 | 0\|0\|9 | 4\|10\|20 | 0\|0\|10 | F = 3.1 d.f. = 2.78 P = 0.0506 |
| CNS.DAF | 0\|3\|7 | 1\|7\|25 | 11\|24.5\|26 | 1\|7\|25 | F = 4.96 d.f. = 2.78 P = 0.00942 |
| COAG.DAF | 1\|5\|8 | 1\|4\|24 | 14.25\|28\|28 | 1\|6\|25 | F = 6.03 d.f. = 2.78 P = 0.00367 |
| INR.DAF | 0\|3\|7 | 1\|4\|21.75 | 14\|26.5\|28 | 0\|5\|22 | F = 7.54 d.f. = 2.78 P = 0.00101 |
| ACRF.DAF | 0\|0\|4 | 1\|5\|20 | 11\|20\|27.75 | 0\|4\|20 | F = 9.11 d.f. = 2.78 P < 0.001 |
| ANYREN.DAF | 0\|0\|4 | 0\|3.5\|19.5 | 11\|20\|27.75 | 0\|4\|20 | P = 8.82 d.f. = 2.78 P < 0.001 |
| RENSUP.DAF | 1\|3\|8 | 1\|3.5\|17.5 | 11\|21.5\|28 | 1\|4\|18 | F = 3.62 d.f. = 2.78 P = 0.0313 |
| ACHEP.DAF | 1\|5\|8 | 1\|5.5\|22 | 14.25\|28\|28 | 1\|6\|28 | F = 3.54 d.f. = 2.78 P = 0.0339 |
| ANYHEP.DAF | 1\|5\|8 | 1\|4.5\|22 | 14.25\|28\|28 | 1\|6\|28 | F = 3.55 d.f. = 2.78 P = 0.0334 |

N, number of subjects.

TABLE 3.17
Difference in response association of leucyl/cystinyl aminopeptidase (LNPEP) rs10051637 and use
of vasopressin between cases (vasopressin-treated group) and controls (vasopressin untreated
matched control) of Caucasian ICU subjects diagnosed with septic shock.

| | rs10051637 GG | | | rs10051637 AG | | | rs10051637 AA | | |
|---|---|---|---|---|---|---|---|---|---|
| | (N = 24) | (N = 10) | | (N = 29) | (N = 46) | | (N = 19) | (N = 25) | |
| | Treat | Cont | DELTA | Treat | Cont | DELTA | Treat | Cont | DELTA |
| SURVIVAL | 46% (11) | 60% (6) | −14% | 38% (11) | 35% (16) | 3% | 26% (5) | 20% (5) | 6% |
| DAYS ALIVE | 15.5 | 28 | −12.5 | 20 | 8 | 12 | 6 | 5 | 1 |
| ALI.DAF | 6.5 | 9.5 | −3 | 10 | 2.5 | 7.5 | 2 | 5 | −3 |
| PRESS.DAF | 6.5 | 23 | −16.5 | 5 | 3.5 | 1.5 | 0 | 2 | −2 |
| PRESS2.DAF | 6.5 | 23 | −16.5 | 5 | 3.5 | 1.5 | 0 | 2 | −2 |
| PRESS5.DAF | 8 | 25 | −17 | 8 | 4 | 4 | 0 | 2 | −2 |
| PRESS15.DAF | 12 | 26.5 | −14.5 | 12 | 5.5 | 6.5 | 1 | 3 | −2 |
| INO.DAF | 12 | 28 | −16 | 15 | 6 | 9 | 4 | 3 | 1 |
| SIRS2.DAF | 0 | 1 | −1 | 1 | 0 | 1 | 0 | 0 | 0 |
| SIRS3.DAF | 3.5 | 3.5 | 0 | 7 | 2 | 5 | 1 | 1 | 0 |
| SIRS4.DAF | 10.5 | 10.5 | 0 | 16 | 6.5 | 9.5 | 4 | 4 | 0 |
| STER.DAF | 2.5 | 17 | −14.5 | 9 | 4 | 5 | 2 | 5 | −3 |
| CVS.DAF | 1.5 | 21.5 | −20 | 1 | 2 | −1 | 0 | 0 | 0 |
| RESP.DAF | 1 | 11 | −10 | 0 | 1 | −1 | 0 | 1 | −1 |
| PF300.DAF | 0 | 1.5 | −1.5 | 0 | 0 | 0 | 0 | 0 | 0 |
| VENT.DAF | 0 | 10 | −10 | 0 | 0 | 0 | 0 | 0 | 0 |
| CNS.DAF | 11.5 | 24.5 | −13 | 13 | 7 | 6 | 5 | 3 | 2 |
| COAG.DAF | 9 | 28 | −19 | 12 | 4 | 8 | 5 | 5 | 0 |
| INR.DAF | 13 | 26.5 | −13.5 | 13 | 4 | 9 | 5 | 3 | 2 |
| ACRF.DAF | 6 | 20 | −14 | 2 | 5 | −3 | 3 | 0 | 3 |
| ANYREN.DAF | 6 | 20 | −14 | 2 | 3.5 | −1.5 | 3 | 0 | 3 |
| RENSUP.DAF | 4.5 | 21.5 | −17 | 6 | 3.5 | 2.5 | 4 | 3 | 1 |
| ACHEP.DAF | 8.5 | 28 | −19.5 | 15 | 5.5 | 9.5 | 3 | 5 | −2 |
| ANYHEP.DAF | 8.5 | 28 | −19.5 | 15 | 4.5 | 10.5 | 3 | 5 | −2 |

### 1.2 Arginine Vasopressin (AVP)

1.2.1 Improved Response to Vasopressin Treatment of Subjects who have the AA or AC Genotype of AVP rs1410713

It is unknown whether SNPs within the AVP gene and those regions immediately upstream and downstream are associated with the response to vasopressin. AVP rs1410713 can be used to predict response to vasopressin in subjects with septic shock using 28-day survival and measures of organ dysfunction as outcome variables. Of 103 vasopressin-treated and 103 matched-control subjects with septic shock, 72 and 81 were respectively genotyped for AVP rs1410713. Baseline characteristics for subjects with genotypes are shown in Table 3.18 and Table 3.19.

TABLE 3.18
Baseline characteristics of a group of vasopressin-treated Caucasian septic-shock subjects by
arginine vasopressin (AVP) rs1410713 genotype. For age and APACHE II score, data is given as
25^{th} percentile|median|75^{th} percentile. For all other variables, data is given as % (N /N total).

| | AA | AC | CC | Combined | Test |
|---|---|---|---|---|---|
| VASOPRESSIN | (N = 8) | (N = 30) | (N = 34) | (N = 72) | Statistic |
| AGE | 50\|66.5\|69 | 39.25\|57.5\|67.5 | 54\|63.5\|71 | 47\|60\|68.5 | F = 1.23 d.f. = 2.69 P = 0.300 |
| GENDER | 75% (6/8) | 63% (19/30) | 88% (30/34) | 76% (55/72) | X\hat{ }2 = 5.49 d.f. = 2 P = 0.0643 |
| APACHE II | 20\|28.5\|34.75 | 20\|26\|30.75 | 28\|32\|40.75 | 25\|30\|37 | F = 5.4 d.f. = 2.69 P = 0.00664 |
| % SURGICAL | 38% (3/8) | 43% (13/30) | 41% (14/34) | 42% (30/72) | X\hat{ }2 = 0.09 d.f. = 2 P = 0.0954 |

N = number of subjects.

TABLE 3.19
Baseline characteristics of a group of Caucasian septic-shock control subjects by arginine
vasopressin (AVP) rs1410713 genotype. For age and APACHE II score, data is given as 25^{th}
percentile|median|75^{th} percentile. For all other variables, data is given as % (N/N total).

| | AA | AC | CC | Combined | Test |
|---|---|---|---|---|---|
| CONTROL | (N = 6) | (N = 35) | (N = 40) | (N = 81) | Statistic |
| AGE | 46\|53\|59.25 | 42\|52\|68 | 45.75\|61\|71.25 | 44\|56\|71.5 | F = 0.72 d.f. = 2.78 P = 0.491 |
| GENDER | 67% (4/6) | 71% (25/35) | 62% (25/40) | 67% (54/81) | X\hat{ }2 = 0.67 d.f. = 2 P = 0.715 |
| APACHE II | 29.5\|31.5\|32.75 | 22\|27\|34 | 26.75\|30.5\|34.75 | 24\|29\|34 | F = 1.11 d.f. = 2.78 P = 0.334 |
| % SURGICAL | 17% (1/6) | 46% (16/35) | 25% (10/40) | 33% (27/81) | X\hat{ }2 = 4.41 d.f. = 2 P = 0.11 |

N, number of subjects.

Tables 3.20, 3.21 and 3.22 contain 28-day survival and organ dysfunction data for septic-shock subjects genotyped for AVP rs1410713. Vasopressin-treated subjects with the AVP rs1410713 AA genotype had a dramatically increased survival (38%) compared to controls (0%) as demonstrated by the positive values in the AVP rs1410713 AA DELTA column in Table 3.22. Furthermore, vasopressin-treated subjects with the AVP rs1410713 AA genotype were observed to have less organ dysfunction as demonstrated by more DAF of organ dysfunction. Vasopressin-treated subjects with AVP rs1410713 AC genotype were also observed to have increased 28-day survival (479c) compared with that of control subjects (37%).

TABLE 3.20
A response association arginine vasopressin (AVP) rs1410713 in a group of Caucasian ICU
septic shock subjects who were treated with vasopressin. For all variables besides 28-day survival,
data is given as 25^{th} percentile|median|75^{th} percentile. For 28-day survival, data is given as %
(N survived/N total).

| | AA | AC | CC | Combined | Test |
|---|---|---|---|---|---|
| VASOPRESSIN | (N = 8) | (N = 30) | (N = 34) | (N = 72) | Statistic |
| SURVIVAL | 38% (3/8) | 47% (14/30) | 32% (11/34) | 39% (28/72) | Chisquare = 1.38 d.f. = 2 P = 0.501 |
| DAYS ALIVE | 5.75\|11\|28 | 9.25\|22.5\|28 | 2\|9\|28 | 3\|14\|28 | F = 1.78 d.f. = 2.69 P = 0.176 |
| ALI.DAF | 0.75\|5.5\|20 | 2\|8.5\|18.5 | 1\|3.5\|16 | 1\|6\|17.25 | F = 0.18 d.f. = 2.69 P = 0.834 |
| PRESS.DAF | 0\|2\|11.25 | 0\|13.5\|18.75 | 0\|0\|17 | 0\|2\|18.25 | F = 1.49 d.f. = 2.69 P = 0.232 |
| PRESS2.DAF | 0\|2\|12 | 0\|14.5\|20.25 | 0\|0\|17 | 0\|2\|18.5 | F = 1.82 d.f. = 2.69 P = 0.170 |
| PRESS5.DAF | 0.75\|2\|12.75 | 0\|15.5\|22 | 0\|0\|18.5 | 0\|2.5\|20.25 | F = 1.99 d.f. = 2.69 P = 0.144 |
| PRESS15.DAF | 1\|5\|17.25 | 2.25\|18.5\|24.75 | 0\|1\|21.75 | 0\|6.5\|23.25 | F = 2.5 d.f. = 2.69 P = 0.0892 |
| INO.DAF | 4.25\|10\|28 | 3\|19.5\|28 | 1.25\|9\|21.75 | 2\|12\|26 | F = 1.57 d.f. = 2.69 P = 0.215 |
| SIRS2.DAF | 0\|0\|1.25 | 0\|1\|3 | 0\|0\|1 | 0\|0\|2.25 | F = 0.74 d.f. = 2.69 P = 0.48 |
| SIRS3.DAF | 2.25\|4.5\|16.5 | 2\|5.5\|11 | 0.25\|2\|7.75 | 0.75\|4\|11.25 | F = 0.8 d.f. = 2.69 P = 0.455 |
| SIRS4.DAF | 4\|9\|22.75 | 6.5\|16\|23.75 | 2\|5.5\|19.75 | 2\|10\|23 | F = 1.04 d.f. = 2.69 P = 0.359 |
| STER.DAF | 2\|5.5\|28 | 2\|9.5\|22 | 0\|2\|15 | 1\|4\|16.75 | F = 2.14 d.f. = 2.69 P = 0.126 |
| CVS.DAF | 0\|1.5\|11.25 | 0\|5.5\|14 | 0\|0\|8 | 0\|0.5\|14 | F = 1.54 d.f. = 2.69 P = 0.221 |
| RESP.DAF | 0\|0\|4.25 | 0\|2\|5.75 | 0\|0\|8 | 0\|0\|6.5 | F = 0.81 d.f. = 2.69 P = 0.449 |
| PF300.DAF | 0\|0\|0.5 | 0\|0\|1.75 | 0\|0\|0 | 0\|0\|1 | F = 1.75 d.f. = 2.69 P = 0.181 |
| VENT.DAF | 0\|0\|3.75 | 0\|0\|5.75 | 0\|0\|8 | 0\|0\|6.5 | F = 0.31 d.f. = 2.69 P = 0.731 |
| CNS.DAF | 5\|9.5\|28 | 3.75\|19\|26.25 | 2\|7\|23 | 2\|11\|24.25 | F = 1.59 d.f. = 2.69 P = 0.211 |
| COAG.DAF | 4.25\|6\|21.25 | 1\|13.5\|26 | 1\|7\|26 | 1\|8\|26 | F = 0.14 d.f. = 2.69 P = 0.867 |
| INR.DAF | 3.75\|7\|25 | 6.25\|19.5\|27.75 | 0.25\|6.5\|23.75 | 2\|9\|2 | F = 2.88 d.f. = 2.69 P = 0.063 |
| ACRF.DAF | 0\|1.5\|2.75 | 0\|8.5\|22.5 | 1\|5\|23 | 0\|5\|19.25 | F = 1.4 d.f. = 2.69 P = 0.254 |
| ANYREN.DAF | 0\|1.5\|2.75 | 0\|8.5\|17.5 | 1\|5\|19.75 | 0\|5\|18.25 | F = 1.34 d.f. = 2.69 P = 0.269 |
| RENSUP.DAF | 1\|2\|10.0 | 3\|11\|26 | 1\|2\|26.75 | 1\|5.5\|25.5 | F = 1.39 d.f. = 2.69 P = 0.256 |
| ACHEP.DAF | 4.25\|10\|23.25 | 3.25\|15.5\|28 | 1\|3\|24.75 | 2\|9.5\|27.25 | F = 1.98 d.f. = 2.69 P = 0.146 |
| ANYHEP.DAF | 4.25\|10\|23.25 | 3.25\|15\|28 | 1\|3\|24.75 | 2\|9.5\|27.25 | F = 2.14 d.f. = 2.69 P = 0.126 |

N, number of subjects.

TABLE 3.21
A response association of arginine vasopressin (AVP) rs1410713 in a matched control group of
Caucasian ICU septic shock subjects who were not treated with vasopressin. For all variables
besides 28-day survival, data is given as 25^{th} percentile|median|75^{th} percentile. For 28-day
survival, data is given as % (N survived/N total).

| | AA | AC | CC | Combined | Test |
|---|---|---|---|---|---|
| CONTROL | (N = 6) | (N = 35) | (N = 40) | (N = 81) | Statistic |
| SURVIVAL | 0% (0/6) | 37% | 35% (14/40) | 33% | Chisquare = 3.28 d.f = 2 P = 0.194 |
| | | (13/35) | | (27/81) | |
| DAYS ALIVE | 1.75\|4.5\|5.75 | 3.5\|10\|28 | 1.75\|8.5\|28 | 3\|8\|28 | F = 2.06 d.f = 2.78 P = 0.134 |
| ALI.DAF | 1\|1\|3.25 | 2\|7\|16.5 | 1\|4.5\|18.5 | 1\|5\|15 | F = 2.06 d.f. = 2.78 P = 0.135 |
| PRESS.DAF | 0\|1.5\|4.5 | 0\|4\|22 | 0\|4.5\|24.25 | 0\|4\|22 | F = 0.95 d.f. = 2.78 P = 0.393 |
| PRESS2.DAF | 0\|1.5\|4.5 | 0\|4\|22 | 0\|4.5\|24.25 | 0\|4\|22 | F = 0.95 d.f. = 2.78 P = 0.392 |
| PRESS5.DAF | 0.5\|2.5\|4.5 | 0\|4\|24 | 0\|6\|25.25 | 0\|4\|23 | F = 0.75 d.f. = 2.78 P = 0.475 |
| PRESS15.DAF | 0.75\|3.5\|4.75 | 1\|6\|26.5 | 0\|7\|26 | 1\|6\|26 | F = 1.13 d.f. = 2.78 P = 0.328 |
| INO.DAF | 1.25\|3.5\|5.75 | 2.5\|8\|28 | 1\|6.5\|25.75 | 2\|6\|28 | F = 1.1 d.f. = 2.78 P = 0.337 |
| SIRS2.DAF | 0\|0\|0 | 0\|0\|2 | 0\|0\|1 | 0\|0\|1 | F = 1.22 d.f. = 2.78 P = 0.301 |
| SIRS3.DAF | 0.25\|1\|1.75 | 0\|2\|8.5 | 1\|2\|6.75 | 0\|2\|7 | F = 0.93 d.f. = 2.78 P = 0.4 |
| SIRS4.DAF | 1\|2\|3.75 | 2.5\|8\|22 | 1\|7\|22.25 | 2\|7\|22 | F = 2.7 d.f. = 2.78 P = 0.0736 |
| STER.DAF | 1.75\|4.5\|5.75 | 1\|6\|28 | 1\|4.5\|12.75 | 1\|5\|21 | F = 1.19 d.f. = 2.78 P = 0.31 |
| CVS.DAF | 0\|1\|2.0 | 0\|3\|18.5 | 0\|3\|20 | 0\|3\|19 | F = 0.9 d.f. = 2.78 P = 0.409 |
| RESP.DAF | 0.25\|1\|2. | 0\|2\|11.5 | 0\|1\|10 | 0\|1\|10 | F = 0.65 d.f. = 2.78 P = 0.526 |
| PF300.DAF | 0\|0.5\|1.75 | 0\|0\|3 | 0\|0\|0 | 0\|0\|1 | F = 2.99 d.f. = 2.78 P = 0.0559 |
| VENT.DAF | 0\|0\|0.75 | 0\|1\|10.5 | 0\|0\|10 | 0\|0\|10 | F = 1.05 d.f. = 2.78 P = 0.353 |
| CNS.DAF | 0.25\|1\|1 | 3\|7\|24.5 | 1\|8.5\|26 | 1\|7\|25 | F = 3.55 d.f. = 2.78 P = 0.0336 |
| COAG.DAF | 1\|2.5\|5.5 | 2.5\|8\|27.5 | 1\|6.5\|24.25 | 1\|6\|25 | F = 1.56 d.f. = 2.78 P = 0.217 |
| INR.DAF | 0\|0.5\|3.25 | 2.5\|7\|24.5 | 0\|6\|23.5 | 1\|5\|22 | F = 2.59 d.f. = 2.78 P = 0.0812 |
| ACRF.DAF | 0\|0\|3 | 1\|4\|21.5 | 0\|5\|21.75 | 0\|4\|20 | F = 2.19 d.f. = 2.78 P = 0.118 |
| ANYREN.DAF | 0\|0\|0 | 1\|4\|21.5 | 0\|4.5\|20.25 | 0\|4\|20 | F = 3.47 d.f. = 2.78 P = 0.0359 |
| RENSUP.DAF | 1\|2.5\|4.75 | 2\|5\|25.5 | 1\|3.5\|18.25 | 1\|4\|18 | F = 1.42 d.f. = 2.78 P = 0.247 |
| ACHEP.DAF | 1.5\|3.5\|5.5 | 2.5\|6\|26 | 1\|7.5\|28 | 1\|6\|28 | F = 1.2 d.f. = 2.78 P = 0.307 |
| ANYHEP.DAF | 1.5\|3.5\|5.5 | 2\|6\|26 | 1\|7.5\|28 | 1\|6\|28 | F = 0.99 d.f = 2.78 P = 0.377 |

N, number of subjects.

TABLE 3.22
Difference in response association of arginine vasopressin (AVP) rs1410713 between cases
(vasopressin-treated group) (Treat) and controls (vasopressin untreated matched control) (Cont) of
Caucasian ICU subjects diagnosed with septic shock. For all variables besides 28-day survival.
data is presented as medians. For 28-day survival, data is presented as % (N survived/N total).

| | AVP rs1410713 CC | | | AVP rs1410713 AC | | | AVP rs1410713 AA | | |
|---|---|---|---|---|---|---|---|---|---|
| | (N = 34) | (N = 40) | Treat − | (N = 30) | (N = 35) | Treat − | (N = 8) | (N = 6) | Treat − |
| | Treat | Cont | Cont | Treat | Cont | Cont | Treat | Cont | Cont |
| SURVIVAL | 32% (11) | 35% (14) | −3% | 47% (14) | 37% (13) | 10% | 38% (3) | 0% (0) | 38% |
| DAYS | 9 | 8.5 | 0.5 | 22.5 | 10 | 12.5 | 11 | 4.5 | 6.5 |
| ALIVE | | | | | | | | | |
| ALI.DAF | 3.5 | 4.5 | −1 | 8.5 | 7 | 1.5 | 5.5 | 1 | 4.5 |
| PRESS.DAF | 0 | 4.5 | −4.5 | 13.5 | 4 | 9.5 | 2 | 1.5 | 0.5 |
| PRESS2.DAF | 0 | 4.5 | −4.5 | 14.5 | 4 | 10.5 | 2 | 1.5 | 0.5 |
| PRESS5.DAF | 0 | 6 | −6 | 15.5 | 4 | 11.5 | 2 | 2.5 | −0.5 |
| PRESS15.DAF | 1 | 7 | −6 | 18.5 | 6 | 12.5 | 5 | 3.5 | 1.5 |
| INO.DAF | 9 | 6.5 | 2.5 | 19.5 | 8 | 11.5 | 10 | 3.5 | 6.5 |
| SIRS2.DAF | 0 | 0 | 0 | 1 | 0 | 1 | 0 | 0 | 0 |
| SIRS3.DAF | 2 | 2 | 0 | 5.5 | 2 | 3.5 | 4.5 | 1 | 3.5 |
| SIRS4.DAF | 5.5 | 7 | −1.5 | 16 | 8 | 8 | 9 | 2 | 7 |
| STER.DAF | 2 | 4.5 | −2.5 | 9.5 | 6 | 3.5 | 5.5 | 4.5 | 1 |
| CVS.DAF | 0 | 3 | −3 | 5.5 | 3 | 2.5 | 1.5 | 1 | 0.5 |
| RESP.DAF | 0 | 1 | −1 | 2 | 2 | 0 | 0 | 1 | −1 |
| PF300.DAF | 0 | 0 | 0 | 0 | 0 | 0 | 0 | 0.5 | −0.5 |
| VENT.DAF | 0 | 0 | 0 | 0 | 1 | −1 | 0 | 0 | 0 |
| CNS.DAF | 7 | 8.5 | −1.5 | 19 | 7 | 12 | 9.5 | 1 | 8.5 |
| COAG.DAF | 7 | 6.5 | 0.5 | 13.5 | 8 | 5.5 | 6 | 2.5 | 3.5 |
| INR.DAF | 6.5 | 6 | 0.5 | 19.5 | 7 | 12.5 | 7 | 0.5 | 6.5 |
| ACRF.DAF | 5 | 5 | 0 | 8.5 | 4 | 4.5 | 1.5 | 0 | 1.5 |
| ANYREN.DAF | 5 | 4.5 | 0.5 | 8.5 | 4 | 4.5 | 1.5 | 0 | 1.5 |
| RENSUP.DAF | 2 | 3.5 | −1.5 | 11 | 5 | 6 | 2 | 2.5 | −0.5 |
| ACHEP.DAF | 3 | 7.5 | −4.5 | 15.5 | 6 | 9.5 | 10 | 3.5 | 6.5 |
| ANYHEP.DAF | 3 | 7.5 | −4.5 | 15 | 6 | 9 | 10 | 3.5 | 6.5 |

N, number of subjects.

1.2.2 Adverse Response to Vasopressin Treatment of Subjects who have the CT Genotype of AVP rs857240 and Improved Response to Vasopressin Treatment of Subjects who have the CC Genotype of AVP rs857240

It was unknown whether SNPs within the AVP gene and those regions immediately upstream and downstream are associated with the response to vasopressin. It was found that AVP rs857240 can be used to predict response to vasopressin in subjects with septic shock using 28-day survival and measures of organ dysfunction as respective primary and secondary outcome variables. Of 103 vasopressin-treated and 103 matched-control subjects with septic shock, 73 and 83 were respectively genotyped for LNPEP rs857240. Baseline characteristics for subjects with genotypes are shown in Table 3.23 and Table 3.24

TABLE 3.23
Baseline characteristics of a group of vasopressin-treated Caucasian septic
shock subjects by arginine vasopressin (AVP) rs857240 genotype. For age and
APACHE II score, data is given as 25^{th} percentile|median|75^{th} percentile.
For all other variables, data is given as % (N/N total).

| | CC | CT | Combined | Test |
|---|---|---|---|---|
| VASOPRESSIN | (N = 56) | (N = 17) | (N = 73) | Statistic |
| AGE | 46.75\|61.5\|68.75 | 39\|56\|68 | 47\|60\|68.5 | F = 0.33 d.f. = 1.71 P = 0.569 |
| GENDER | 73% (41/56) | 88% (15/17) | 77% (56/73) | X\hat{ }2 = 1.65 d.f. = 1 P = 0.199 |
| APACHE II | 25\|30.5\|36.25 | 24\|28\|39 | 25\|30\|37 | F = 0.09 d.f. = 1.71 P = 0.761 |
| % SURGICAL | 41% (23/56) | 35% (6/17) | 40% (29/73) | X\hat{ }2 = 0.18 d.f. = 1 P = 0.67 |

N, number of subjects.

TABLE 3.24
Baseline characteristics of Caucasian septic shock control subjects by arginine
vasopressin (AVP) rs857240 genotype. For age and APACHE II score, data is
given as 25^{th} percentile|median|75^{th} percentile. For all other
variables, data is given as % (N/N total).

| | CC | CT | Combined | Test |
|---|---|---|---|---|
| CONTROL | (N = 69) | (N = 14) | (N = 83) | Statistic |
| AGE | 44\|55\|68 | 36.75\|53.5\|71 | 44\|56\|71.5 | F = 0.12 d.f. = 1.81 P = 0.731 |
| GENDER | 65% (45/69) | 79% (11/14) | 67% (56/83) | X\hat{ }2 = 0.95 d.f. = 1 P = 0.331 |
| APACHE II | 25\|29\|34 | 27\|32\|34 | 24\|29\|34 | F = 0.59 d.f. = 1.81 P = 0.446 |
| % SURGICAL | 35% (24/69) | 29% (4/14) | 34% (28/83) | X\hat{ }2 = 0.2 d.f. = 1 P = 0.654 |

N, number of subjects.

Tables 3.25, 3.26 and 3.27 contain 28-day survival and organ dysfunction data for septic-shock subjects genotyped for AVP rs857240. Vasopressin-treated subjects with the AVP rs857240 CT genotype had dramatically decreased survival if vasopressin-treated (29%) compared to controls (43%) as demonstrated by the negative values in the AVP rs857240 CT DELTA column in Table 3.27. Furthermore, vasopressin-treated subjects with the AVP rs857240 CT genotype were observed to have more organ dysfunction than AVP rs857240 CT control subjects as demonstrated by more DAF of organ dysfunction. In contrast, vasopressin-treated subjects with the AVP rs857240 CC genotype had increased survival (41%) compared to controls (30%) as demonstrated by the positive values in the AVP rs857240 CC DELTA column in Table 3.27. Furthermore, vasopressin-treated subjects AVP rs857240CC subjects were observed to have less organ dysfunction than AVP rs857240 CC control subjects.

TABLE 3.25
A response association of arginine vasopressin (AVP) rs857240 in a group
of Caucasian ICU septic shock subjects who were treated with vasopressin.
For all variables besides 28-day survival, data is given as
25^{th} percentile|median|75^{th} percentile. For 28-day survival,
data is given as % (N survived/N total).

| | CC | CT | Combined | Test |
|---|---|---|---|---|
| VASOPRESSIN | (N = 56) | (N = 17) | (N = 73) | Statistic |
| SURVIVAL | 41% (23/56) | 29% (5/17) | 38% (28/73) | Chisquare = 0.75 d.f. = 1 P = 0.387 |
| DAYS ALIVE | 5.75\|19.5\|28 | 2\|5\|28 | 3\|13\|28 | F = 2.96 d.f. = 1.71 P = 0.0899 |
| ALI.DAF | 2\|6\|17 | 1\|3\|9 | 1\|6\|17 | F = 1.26 d.f. = 1.71 P = 0.265 |
| PRESS.DAF | 0\|7.5\|19 | 0\|0\|5 | 0\|1\|19 | F = 2.66 d.f. = 1.71 P = 0.108 |
| PRESS2.DAF | 0\|8\|20.25 | 0\|0\|5 | 0\|1\|20 | F = 2.1 d.f. = 1.71 P = 0.151 |
| PRESS5.DAF | 0\|10.5\|21.25 | 0\|0\|7 | 0\|2\|21 | F = 2.54 d.f. = 1.71 P = 0.116 |
| PRESS15.DAF | 0\|14\|24 | 0\|1\|11 | 0\|6\|23 | F = 3.01 d.f. = 1.71 P = 0.087 |
| INO.DAF | 2\|13.5\|28 | 1\|4\|22 | 2\|12\|26 | F = 2.51 d.f. = 1.71 P = 0.118 |
| SIRS2.DAF | 0\|0\|2.25 | 0\|0\|1 | 0\|0\|2 | F = 0.18 d.f. = 1.71 P = 0.671 |
| SIRS3.DAF | 1\|4\|11.5 | 0\|2\|7 | 1\|4\|11 | F = 1.56 d.f. = 1.71 P = 0.216 |
| SIRS4.DAF | 3\|15\|22.25 | 2\|3\|20 | 2\|10\|22 | F = 1.52 d.f. = 1.71 P = 0.221 |
| STER.DAF | 1\|5\|21 | 0\|3\|11 | 1\|4\|19 | F = 0.58 d.f. = 1.71 P = 0.448 |
| CVS.DAF | 0\|2.5\|14.25 | 0\|0\|3 | 0\|0\|14 | F = 1.97 d.f. = 1.71 P = 0.165 |
| RESP.DAF | 0\|0\|8 | 0\|0\|2 | 0\|0\|8 | F = 0.19 d.f. = 1.71 P = 0.661 |
| PF300.DAF | 0\|0\|1.25 | 0\|0\|0 | 0\|0\|1 | F = 1.43 d.f. = 1.71 P = 0.235 |
| VENT.DAF | 0\|0\|8 | 0\|0\|2 | 0\|0\|8 | F = 0 d.f. = 1.71 P = 0.946 |
| CNS.DAF | 3\|13\|25 | 2\|5\|21 | 2\|11\|24 | F = 2.4 d.f. = 1.71 P = 0.126 |
| COAG.DAF | 1.75\|9.5\|26 | 1\|3\|18 | 1\|8\|26 | F = 1.56 d.f. = 1.71 P = 0.216 |
| INR.DAF | 2\|14\|27 | 1\|4\|20 | 2\|8\|27 | F = 1.95 d.f. = 1.71 P = 0.167 |
| ACRF.DAF | 0\|6\|19.25 | 0\|3\|5 | 0\|5\|19 | F = 0.62 d.f. = 1.71 P = 0.435 |
| ANYREN.DAF | 0\|6\|19 | 0\|3\|5 | 0\|5\|18 | F = 0.98 d.f. = 1.71 P = 0.325 |
| RENSUP.DAF | 1.75\|7.5\|27.25 | 1\|2\|5 | 1\|5\|2 | F = 2.74 d.f. = 1.71 P = 0.102 |
| ACHEP.DAF | 2\|11.5\|27.25 | 2\|3\|16 | 2\|9\|25 | F = 1.41 d.f. = 1.71 P = 0.239 |
| ANYHEP.DAF | 2\|11.5\|27.25 | 1\|3\|15 | 2\|9\|25 | F = 1.7 d.f. = 1.71 P = 0.197 |

N, number of subjects.
Note:
TT genotype frequency = 0.

TABLE 3.26
A response association of arginine vasopressin (AVP) rs857240 a matched control group of
Caucasian ICU septic shock subjects who were not treated with vasopressin. For all variables
besides 28-day survival, data is given as 25^{th} percentile|median|75^{th} percentile. For 28-day
survival, data is given as % (N survived/N total).

| | CC | CT | Combined | Test |
|---|---|---|---|---|
| CONTROL | (N = 69) | (N = 14) | (N = 83) | Statistic |
| SURVIVAL | 30% (21/69) | 43% (6/14) | 33% (27/83) | Chisquare = 0.82 d.f. = 1 P = 0.366 |
| DAYS ALIVE | 3\|7\|28 | 2\|16.5\|28 | 3\|8\|28 | F = 0.16 d.f. = 1.81 P = 0.694 |
| ALI.DAF | 1\|5\|11 | 1.25\|2\|21.75 | 1\|5\|14.5 | F = 0 d.f. = 1.81 P = 0.995 |
| PRESS.DAF | 0\|3\|19 | 0\|12.5\|23.75 | 0\|4\|22 | F = 0.49 d.f. = 1.81 P = 0.487 |
| PRESS2.DAF | 0\|3\|19 | 0\|12.5\|23.75 | 0\|4\|22 | F = 0.45 d.f. = 1.81 P = 0.503 |
| PRESS5.DAF | 0\|4\|21 | 0\|12.5\|24.5 | 0\|4\|23 | F = 0.43 d.f. = 1.81 P = 0.516 |
| PRESS15.DAF | 1\|5\|26 | 0\|15\|25.75 | 0.5\|5\|26 | F = 0.05 d.f. = 1.81 P = 0.817 |
| INO.DAF | 1\|5\|25 | 2\|13\|28 | 2\|6\|28 | F = 0.4 d.f. = 1.81 P = 0.53 |
| SIRS2.DAF | 0\|0\|1 | 0\|0\|1.75 | 0\|0\|1 | F = 0.11 d.f. = 1.81 P = 0.744 |
| SIRS3.DAF | 0\|2\|6 | 0.25\|2\|16 | 0\|2\|6.5 | F = 0.41 d.f. = 1.81 P = 0.524 |
| SIRS4.DAF | 2\|6\|17 | 1.25\|14\|24.75 | 2\|6\|21.5 | F = 0.16 d.f. = 1.81 P = 0.694 |
| STER.DAF | 1\|5\|19 | 1\|2\|18.25 | 1\|5\|20 | F = 0.19 d.f. = 1.81 P = 0.666 |
| CVS.DAF | 0\|2\|18 | 0\|8\|22 | 0\|2\|18.5 | F = 0.64 d.f. = 1.81 P = 0.425 |
| RESP.DAF | 0\|1\|9 | 0\|3\|18.25 | 0\|1\|9.5 | F = 0.87 d.f. = 1.81 P = 0.354 |
| PF300.DAF | 0\|0\|2 | 0\|0\|1 | 0\|0\|1 | F = 0.06 d.f. = 1.81 P = 0.81 |
| VENT.DAF | 0\|0\|9 | 0\|3\|18.25 | 0\|0\|9.5 | F = 1.63 d.f. = 1.81 P = 0.205 |
| CNS.DAF | 1\|6\|24 | 1.25\|15\|25.75 | 1\|7\|25 | F = 0.47 d.f. = 1.81 P = 0.497 |
| COAG.DAF | 1\|6\|24 | 1.25\|7.5\|28 | 1\|6\|24.5 | F = 0.34 d.f. = 1.81 P = 0.563 |
| INR.DAF | 1\|4\|14 | 0\|15.5\|24.25 | 0\|4\|21.5 | F = 0.03 d.f. = 1.81 P = 0.855 |
| ACRF.DAF | 0\|4\|15 | 1.25\|9\|26.75 | 0\|4\|20 | F = 1.6 d.f. = 1.81 P = 0.21 |
| ANYREN.DAF | 0\|3\|15 | 1\|9\|24.75 | 0\|3\|19 | F = 1.39 d.f. = 1.81 P = 0.242 |
| RENSUP.DAF | 1\|4\|15 | 1.25\|5.5\|26.25 | 1\|4\|17 | F = 0.52 d.f. = 1.81 P = 0.475 |
| ACHEP.DAF | 1\|6\|22 | 1.25\|16.5\|28 | 1\|6\|26 | F = 0.65 d.f. = 1.81 P = 0.424 |
| ANYHEP.DAF | 1\|5\|22 | 1.25\|16.5\|28 | 1\|6\|26 | F = 1.01 d.f. = 1.81 P = 0.319 |

N, number of subjects.
Note:
TT genotype frequency = 0.

TABLE 3.27
Difference in response association of arginine vasopressin (AVP) rs857240
between cases (vasopressin-treated group) (Treat) and controls (vasopressin
untreated matched control) (Cont) of Caucasian ICU subjects diagnosed with
septic shock. For all variables besides 28-day survival, data is presented as
medians. For 28-day survival, data is presented as % (N survived/N total).

| | rs857240 CT | | | rs857240 CC | | |
|---|---|---|---|---|---|---|
| | (N = 17) | (N = 14) | Treat − | (N = 56) | (N = 69) | Treat − |
| | Treat | Cont | Cont | Treat | Cont | Cont |
| SURVIVAL | 29% (5/17) | 43% (6/14) | −14% | 41% (23/56) | 30% (21/69) | 11% |
| DAYS ALIVE | 5 | 16.5 | −11.5 | 19.5 | 7 | 12.5 |
| ALI.DAF | 3 | 2 | 1 | 6 | 5 | 1 |
| PRESS.DAF | 0 | 12.5 | −12.5 | 7.5 | 3 | 4.5 |
| PRESS2.DAF | 0 | 12.5 | −12.5 | 8 | 3 | 5 |
| PRESS5.DAF | 0 | 12.5 | −12.5 | 10.5 | 4 | 6.5 |
| PRESS15.DAF | 1 | 15 | −14 | 14 | 5 | 9 |
| INO.DAF | 4 | 13 | −9 | 13.5 | 5 | 8.5 |
| SIRS2.DAF | 0 | 0 | 0 | 0 | 0 | 0 |
| SIRS3.DAF | 2 | 2 | 0 | 4 | 2 | 2 |
| SIRS4.DAF | 3 | 14 | −11 | 15 | 6 | 9 |
| STER.DAF | 3 | 2 | 1 | 5 | 5 | 0 |
| CVS.DAF | 0 | 8 | −8 | 2.5 | 2 | 0.5 |
| RESP.DAF | 0 | 3 | −3 | 0 | 1 | −1 |
| PF300.DAF | 0 | 0 | 0 | 0 | 0 | 0 |
| VENT.DAF | 0 | 3 | −3 | 0 | 0 | 0 |
| CNS.DAF | 5 | 15 | −10 | 13 | 6 | 7 |
| COAG.DAF | 3 | 7.5 | −4.5 | 9.5 | 6 | 3.5 |
| INR.DAF | 4 | 15.5 | −11.5 | 14 | 4 | 10 |
| ACRF.DAF | 3 | 9 | −6 | 6 | 4 | 2 |
| ANYREN.DAF | 3 | 9 | −6 | 6 | 3 | 3 |
| RENSUP.DAF | 2 | 5.5 | −3.5 | 7.5 | 4 | 3.5 |
| ACHEP.DAF | 3 | 16.5 | −13.5 | 11.5 | 6 | 5.5 |
| ANYHEP.DAF | 3 | 16.5 | −13.5 | 11.5 | 5 | 6.5 |

N, number of subjects.
Note:
TT genotype frequency = 0.

1.2.3 Adverse Response to Vasopressin Treatment of Subjects who have the AC Genotype of AVP rs857242 and Improved Response to Vasopressin Treatment of Subjects who have the CC Genotype of AVP rs857242

It was unknown whether SNPs within the AVP gene and those regions immediately upstream and downstream are associated with the response to vasopressin. It was found that AVP rs857242 can be used to predict response to vasopressin in subjects with septic shock using 28-day survival and measures of organ dysfunction as respective primary and secondary outcome variables. Of 103 vasopressin-treated and 103 matched-control subjects with septic shock, 75 and 81 were respectively genotyped for AVP rs857242. Baseline characteristics for subjects with genotypes are shown in Table 3.28 and Table 3.29.

TABLE 3.28
Baseline characteristics of a group of vasopressin-treated Caucasian ICU septic
shock subjects by genotype of arginine vasopressin (AVP) rs 857242. For age and
APACHE II score, data is given as 25^{th} percentile|median|75^{th} percentile.
For all other variables, data is given as % (N/N total).

| | AC | CC | Combined | Test |
|---|---|---|---|---|
| VASOPRESSIN | (N = 16) | (N = 59) | (N = 75) | Statistic |
| AGE | 39.75\|60\|68.75 | 46.5\|61\|69.5 | 47\|60\|68.5 | F = 0.09 d.f. = 1.73 P = 0.763 |
| GENDER | 94% (15/16) | 73% (43/59) | 77% (58/75) | X\hat{ }2 = 3.13 d.f. = P = 0.077 |
| APACHE II | 24.75\|28\|39.5 | 25\|30\|35 | 25\|30\|37 | F = 0 d.f. = 1.73 P = 0.96 |
| % SURGICAL | 38% (6/16) | 41% (24/59) | 40% (30/75) | X\hat{ }2 = 0.05 d.f. = 1 P = 0.818 |

N, number of subjects.

TABLE 3.29
Baseline characteristics of a vasopressin untreated matched control group of Caucasian ICU septic
shock subjects by genotype of arginine vasopressin (AVP) rs 857242. For age and APACHE II
score, data is given as 25^{th} percentile|median|75^{th} percentile. For all other variables, data is
given as % (N/N total).

| | AA | AC | CC | Combined | Test |
|---|---|---|---|---|---|
| CONTROL | (N = 1) | (N = 13) | (N = 67) | (N = 81) | Statistic |
| AGE | 72\|72\|72 | 39\|48\|65 | 43.5\|55\|70 | 44\|56\|71.5 | F = 0.98 d.f. = 2.78 P = 0.38 |
| GENDER | 0% (0/1) | 69% (9/13) | 69% (46/67) | 68% (55/81) | X\hat{ }2 = 2.14 d.f. = 2 P = 0.342 |
| APACHE II | 19\|19\|19 | 23\|30\|34 | 25.5\|29\|34 | 24\|29\|34 | F = 1.03 d.f. = 2.78 P = 0.361 |
| % SURGICAL | 0% (0/1) | 38% (5/13) | 34% (23/67) | 35% (28/81) | X\hat{ }2 = 0.62 d.f. = 2 P = 0.734 |

N, number of subjects.

Tables 3.30, 3.31 and 3.32 contain 28-day survival and organ dysfunction data for septic-shock subjects genotyped for AVP rs857242. Vasopressin-treated subjects with the AVP rs857242 AC genotype had a dramatically decreased survival (38%) compared to controls (54%) as demonstrated by the negative values in the AVP rs857242 AC DELTA column in Table 3.32. Furthermore, vasopressin-treated subjects with the AVP rs857242 AC genotype were observed to have more organ dysfunction as demonstrated by more DAF of organ dysfunction. In contrast, vasopressin-treated subjects with the AVP rs857242 CC genotype were observed to have increased survival (417c) compared with controls (301). As well, vasopressin-treated subjects with AVP rs857242 CC genotype were observed to have increased 28-day survival (47%) compared with that of control subjects (37%) as demonstrated by the positive values in the AVP rs857242 CC DELTA column in Table 3.32. Furthermore, vasopressin-treated subjects with the AVP rs857242 CC genotype were observed to have less organ dysfunction as demonstrated by more DAF of organ dysfunction

TABLE 3.30
A response association of arginine vasopressin (AVP) rs857242 in a group of
Caucasian ICU septic shock subjects who were treated with vasopressin. For all
variables besides 28-day survival, data is given as 25^{th} percentile|median|75^{th}
percentile. For 28-day survival, data is given as % (N survived/N total).

| | AC | CC | Combined | Test |
|---|---|---|---|---|
| VASOPRESSIN | (N = 16) | (N = 59) | (N = 75) | Statistic |
| SURVIVAL | 38% (6/16) | 41% (24/59) | 40% (30/75) | Chisquare = 0.05 d.f. = 1 P = 0.818 |
| DAYS ALIVE | 2.75\|7.5\|28 | 5\|19\|28 | 3\|15\|28 | F = 0.96 d.f. = 1.73 P = 0.332 |
| ALI.DAF | 1\|6.5\|17.25 | 2\|6\|18.5 | 1\|6\|17.5 | F = 0.4 d.f. = 1.73 P = 0.528 |
| PRESS.DAF | 0\|0\|18.75 | 0\|7\|19 | 0\|3\|19 | F = 1.65 d.f. = 1.73 P = 0.204 |
| PRESS2.DAF | 0\|0\|18.75 | 0\|7\|20.5 | 0\|3\|20.5 | F = 1.22 d.f. = 1.73 P = 0.273 |
| PRESS5.DAF | 0\|0\|19.5 | 0\|10\|21.5 | 0\|3\|21 | F = 1.55 d.f. = 1.73 P = 0.217 |
| PRESS15.DAF | 0\|1.5\|21 | 0\|14\|24 | 0\|7\|23.5 | F = 1.81 d.f. = 1.73 P = 0.182 |
| INO.DAF | 1\|6\|24.5 | 2\|13\|27.5 | 2\|12\|26.5 | F = 0.96 d.f. = 1.73 P = 0.331 |
| SIRS2.DAF | 0\|0.5\|3 | 0\|0\|2 | 0\|0\|2.5 | F = 0.06 d.f. = 1.73 P = 0.802 |
| SIRS3.DAF | 0\|2.5\|9.75 | 1\|4\|12 | 1\|4\|11.5 | F = 0.19 d.f. = 1.73 P = 0.66 |
| SIRS4.DAF | 2\|6.5\|23.25 | 2.5\|14\|22.5 | 2\|10\|23 | F = 0.23 d.f. = 1.73 P = 0.635 |
| STER.DAF | 0\|3.5\|17.25 | 1\|5\|19.5 | 1\|4\|19.5 | F = 0.08 d.f. = 1.73 P = 0.776 |
| CVS.DAF | 0\|0\|8 | 0\|3\|14.5 | 0\|1\|14 | F = 1.21 d.f. = 1.73 P = 0.276 |
| RESP.DAF | 0\|0.5\|11 | 0\|0\|7 | 0\|0\|8 | F = 0.04 d.f. = 1.73 P = 0.835 |
| PF300.DAF | 0\|0\|0.25 | 0\|0\|1 | 0\|0\|1 | F = 0.19 d.f. = 1.73 P = 0.667 |
| VENT.DAF | 0\|0\|9.25 | 0\|0\|7 | 0\|0\|8 | F = 0.23 d.f. = 1.73 P = 0.632 |
| CNS.DAF | 2\|6.5\|24 | 3\|13\|25 | 2\|11\|25 | F = 0.89 d.f. = 1.73 P = 0.349 |
| COAG.DAF | 0.75\|3.5\|20.75 | 1.5\|9\|26.5 | 1\|8\|26 | F = 0.7 d.f. = 1.73 P = 0.407 |
| INR.DAF | 1.75\|5.5\|24.25 | 2\|13\|27 | 2\|10\|27 | F = 0.61 d.f. = 1.73 P = 0.438 |
| ACRF.DAF | 0\|3.5\|16.25 | 0.5\|6\|22 | 0\|5\|22 | F = 0.4 d.f. = 1.73 P = 0.529 |
| ANYREN.DAF | 0\|3.5\|12.25 | 0.5\|6\|$$9 | 0\|5\|19 | F = 0.72 d.f. = 1.73 P = 0.399 |
| RENSUP.DAF | 1\|2\|12.25 | 2\|6\|28 | 1\|6\|27 | F = 2.25 d.f. = 1.73 P = 0.138 |
| ACHEP.DAF | 1.75\|3.5\|18.25 | 2\|10\|27.5 | 2\|9\|26 | F = 0.57 d.f. = 1.73 P = 0.453 |
| ANYHEP.DAF | 1.75\|3.5\|18.25 | 2\|10\|27.5 | 2\|9\|26 | F = 0.48 d.f. = 1.73 P = 0.493 |

N, number of subjects.
Note:
AA genotype frequency = 0.

TABLE 3.31
A response association of arginine vasopressin (AVP) rs857242 in Caucasian
septic-shock control subjects. For all variables besides 28-day survival, data is
given as 25^{th} percentile|median|75^{th} percentile. For 28-day survival,
data is given as % (N survived/N total).

| | AC | CC | Combined | Test |
|---|---|---|---|---|
| CONTROL | (N = 13) | (N = 67) | (N = 80) | Statistic |
| SURVIVAL | 54% (7/13) | 30% (20/67) | 34% (27/80) | Chisquare = 2.8 d.f. = 1 P = 0.094 |
| DAYS ALIVE | 4\|28\|28 | 2.5\|7\|28 | 3\|8\|28 | F = 1.67 d.f. = 1.78 P = 0.199 |
| ALI.DAF | 1\|4\|22 | 1\|5\|12.5 | 1\|5\|15.75 | F = 0.35 d.f. = 1.78 P = 0.554 |
| PRESS.DAF | 1\|17\|25 | 0\|3\|18.5 | 0\|4\|23 | F = 1.9 d.f. = 1.78 P = 0.172 |
| PRESS2.DAF | 2\|17\|26 | 0\|3\|18.5 | 0\|4\|23 | F = 2.1 d.f. = 1.78 P = 0.152 |
| PRESS5.DAF | 4\|20\|26 | 0\|4\|20.5 | 0\|4\|23.5 | F = 2.21 d.f. = 1.78 P = 0.141 |
| PRESS15.DAF | 4\|24\|28 | 0.5\|5\|25 | 0.75\|5.5\|26 | F = 1.67 d.f. = 1.78 P = 0.201 |
| INO.DAF | 4\|20\|28 | 1\|5\|28 | 1.75\|6\|28 | F = 1.51 d.f. = 1.78 P = 0.287 |
| SIRS2.DAF | 0\|2\|13 | 0\|0\|1 | 0\|0\|1 | F = 4.68 d.f. = 1.78 P = 0.0335 |
| SIRS3.DAF | 2\|4\|22 | 0\|1\|5 | 0\|2\|6.25 | F = 4.99 d.f. = 1.78 P = 0.0284 |
| SIRS4.DAF | 4\|22\|27 | 2\|5\|16 | 2\|6.5\|22 | F = 3.23 d.f. = 1.78 P = 0.0761 |
| STER.DAF | 1\|6\|26 | 1\|5\|17 | 1\|5\|21.75 | F = 0.09 d.f. = 1.78 P = 0.769 |
| CVS.DAF | 0\|11\|23 | 0\|2\|18 | 0\|2.5\|19 | F = 1.58 d.f. = 1.78 P = 0.212 |
| RESP.DAF | 0\|4\|19 | 0\|1\|9 | 0\|1\|9.25 | F = 0.13 d.f. = 1.78 P = 0.722 |
| PF300.DAF | 0\|0\|0 | 0\|0\|1.5 | 0\|0\|1 | F = 0.79 d.f. = 1.78 P = 0.376 |
| VENT.DAF | 0\|4\|19 | 0\|0\|9 | 0\|0\|9.25 | F = 0.75 d.f. = 1.78 P = 0.39 |
| CNS.DAF | 4\|22\|28 | 1\|5\|24 | 1\|7\|25 | F = 3.3 d.f. = 1.78 P = 0.0732 |
| COAG.DAF | 3\|12\|28 | 1\|6\|24 | 1\|6\|25.5 | F = 1.7 d.f. = 1.78 P = 0.197 |
| INR.DAF | 4\|14\|26 | 0\|4\|18 | 0.75\|4.5\|23.5 | F = 1.91 d.f. = 1.78 P = 0.171 |
| ACRF.DAF | 1\|7\|28 | 0\|4\|17.5 | 0\|4\|20.75 | F = 3.05 d.f. = 1.78 P = 0.0844 |
| ANYREN.DAF | 1\|7\|27 | 0\|3\|17.5 | 0\|3.5\|20 | F = 1.2 d.f. = 1.78 P = 0.278 |
| RENSUP.DAF | 1\|9\|28 | 1\|4\|14.5 | 1\|4\|16.5 | F = 0.49 d.f. = 1.78 P = 0.488 |
| ACHEP.DAF | 4\|22\|28 | 1\|6\|21.5 | 1\|6\|28 | F = 2.9 d.f. = 1.78 P = 0.0926 |
| ANYHEP.DAF | 4\|22\|28 | 1\|5\|21.5 | 1\|6\|28 | F = 3.27 d.f. = 1.78 P = 0.0745 |

N, number of subjects.
Note:
AA genotype frequency = 0.

TABLE 3.32
Difference in response association of arginine vasopressin (AVP) rs857242 between cases
(vasopressin-treated group) (Treat) and controls (vasopressin untreated matched control)
(Cont) of Caucasian ICU subjects diagnosed with septic shock. For all variables besides
28-day survival, data is presented as medians. For 28-day survival, data is presented
as %(N survived/N total). N, number of subjects.

| | rs857242 AC | | | rs857242 CC | | |
|---|---|---|---|---|---|---|
| | (N = 16) | (N = 13) | | (N = 59) | (N = 67) | |
| | Treat | Cont | DELTA | Treat | Cont | DELTA |
| SURVIVAL | 38% (6/16) | 54% (7/13) | −16% | 41% (24/59) | 30% (20/67) | 11% |
| DAYS ALIVE | 7.5 | 28 | −20.5 | 19 | 7 | 12 |
| ALI.DAF | 6.5 | 4 | 2.5 | 6 | 5 | 1 |
| PRESS.DAF | 0 | 17 | −17 | 7 | 3 | 4 |
| PRESS2.DAF | 0 | 17 | −17 | 7 | 3 | 4 |
| PRESS5.DAF | 0 | 20 | −20 | 10 | 4 | 6 |
| PRESS15.DAF | 1.5 | 24 | −22.5 | 14 | 5 | 9 |
| INO.DAF | 6 | 20 | −14 | 13 | 5 | 8 |
| SIRS2.DAF | 0.5 | 2 | −1.5 | 0 | 0 | 0 |
| SIRS3.DAF | 2.5 | 4 | −1.5 | 4 | 1 | 3 |
| SIRS4.DAF | 6.5 | 22 | −15.5 | 14 | 5 | 9 |
| STER.DAF | 3.5 | 6 | −2.5 | 5 | 5 | 0 |
| CVS.DAF | 0 | 11 | −11 | 3 | 2 | 1 |
| RESP.DAF | 0.5 | 4 | −3.5 | 0 | 1 | −1 |
| PF300.DAF | 0 | 0 | 0 | 0 | 0 | 0 |
| VENT.DAF | 0 | 4 | −4 | 0 | 0 | 0 |
| CNS.DAF | 6.5 | 22 | −15.5 | 13 | 5 | 8 |
| COAG.DAF | 3.5 | 12 | −8.5 | 9 | 6 | 3 |
| INR.DAF | 5.5 | 14 | −8.5 | 13 | 4 | 9 |
| ACRF.DAF | 3.5 | 7 | −3.5 | 6 | 4 | 2 |
| ANYREN.DAF | 3.5 | 7 | −3.5 | 6 | 3 | 3 |
| RENSUP.DAF | 2 | 9 | −7 | 6 | 4 | 2 |
| ACHEP.DAF | 3.5 | 22 | −18.5 | 10 | 6 | 4 |
| ANYHEP.DAF | 3.5 | 22 | −18.5 | 10 | 5 | 5 |

Note:
AA genotype frequency = 0.

### 1.3 Arginine Vasopressin Receptor 1a (AVPR1A)

1.3.1 Adverse Response to Vasopressin Treatment of Subjects who have the TT Genotype of AVPR1A rs1495027 and Improved Response to Vasopressin Treatment of Subjects who have the CC Genotype of AVPR1A rs1495027

It was unknown whether SNPs within the AVPR1A gene and those regions immediately upstream and downstream are associated with the response to vasopressin. It was found that AVPR1A rs1495027 can be used to predict response to vasopressin in subjects with septic shock using 28-day survival and measures of organ dysfunction as respective primary and secondary outcome variables. Of 103 vasopressin-treated and 103 matched-control subjects with septic shock. 72 and 79 were respectively genotyped for AVPR1A rs1495027. Baseline characteristics for subjects with genotypes are shown in Table 3.33 and Table 3.34.

TABLE 3.33
Baseline characteristics of a group of vasopressin-treated Caucasian ICU septic shock subjects by
genotype of arginine vasopressin receptor 1a (AVPR1A) rs1495027. For age and APACHE II
score, data is given as 25^{th} percentile|median|75^{th} percentile. For all other variables, data is
given as % (N/N total).

| | CC | CT | TT | Combined | Test |
|---|---|---|---|---|---|
| VASOPRESSIN | (N = 14) | (N = 45) | (N = 13) | (N = 72) | Statistic |
| AGE | 57\|67\|72 | 42\|55\|66 | 39\|65\|71 | 47\|60\|68 | F = 2.6 d.f. = 2.69 P = 0.0816 |
| GENDER | 79% (11/14) | 80% (36/66) | 62% (8/13) | 76% (55/72) | X\hat{ }2 = 1.95 d.f. = 2 P = 0.377 |
| APACHE II | 23.75\|30\|33.75 | 25\|31\|37 | 25\|30\|40 | 25\|30\|37 | F = 0.12 d.f. = 2.69 P = 0.889 |
| % SURGICAL | 50% (7/14) | 40% (18/66) | 31% (4/13) | 40% (29/72) | X\hat{ }2 = 1.04 d.f. = 2 P = 0.594 |

N, number of subjects.

TABLE 3.34
Baseline characteristics of a vasopressin untreated matched control group of Caucasian ICU septic
shock subjects by genotype of arginine vasopressin receptor 1a (AVPR1A) rs1495027. For age
and APACHE II score, data is given as 25^{th} percentile|median|75^{th} percentile. For all other
variables, data is given as % (N/N total).

| | CC | CT | TT | Combined | Test |
|---|---|---|---|---|---|
| CONTROL | (N = 29) | (N = 37) | (N = 13) | (N = 79) | Statistic |
| AGE | 44\|57\|68 | 43\|52\|67 | 49\|64\|72 | 44\|56\|71.5 | F = 0.68 d.f. = 2.76 P = 0.51 |
| GENDER | 52% (15/29) | 76% (28/37) | 77% (10/13) | 67% (53/79) | X\hat{ }2 = 4.91 d.f. = 2 P = 0.086 |
| APACHE II | 27\|31\|33 | 25\|29\|34 | 29\|34\|37 | 24\|29\|34 | F = 1.06 d.f. 2.76 P = 0.351 |
| % SURGICAL | 24% (7/29) | 32% (12/37) | 54% (7/13) | 33% (26/79) | X\hat{ }2 = 3.6 d.f. = 2 P = 0.166 |

N, number of subects.

Tables 3.35, 3.36 and 3.37 contain 28-day survival and organ dysfunction data for septic-shock subjects genotyped for AVPR1A rs1495027. Vasopressin-treated subjects with the AVPR1A rs1495027 TT had a dramatically decreased survival (23%) compared to controls (46%) as demonstrated by the negative values in the AVPR1A rs1495027 TT DELTA column in Table 3.37. Furthermore, vasopressin-treated subjects with the AVPR1A rs1495027 TT genotype were observed to have more organ dysfunction as demonstrated by fewer DAF of organ dysfunction. In contrast, vasopressin-treated subjects with the AVPR1A rs1495027 CC genotype were shown to have increased survival (50%) over AVPR1A rs1495027 CC controls (24%) as demonstrated by the positive values in the AVPR1A rs1495027 TT DELTA column in Table 3.37. In addition, vasopressin subjects with the AVPR1A rs1495027 CC genotype had less organ dysfunction as evidenced by more DAF of organ dysfunction.

TABLE 3.35
A response association of AVPR1A rs 1495027 in vasopressin-treated Caucasian septic-shock
subjects. For all variables besides 28-day survival, data is given as 25^{th} percentile|
median|75^{th} percentile. For 28-day survival, data is given as % (N survived/N total).

| | CC | CT | TT | Combined | Test |
|---|---|---|---|---|---|
| VASOPRESSIN | (N = 14) | (N = 45) | (N = 13) | (N = 72) | Statistic |
| SURVIVAL | 50% (7/14) | 38% (17/45) | 23% (3/13) | 38% (27/72) | Chisquare = 2.09 d.f. = 2 P = 0.352 |
| DAYS ALIVE | 3.75\|18.5\|28 | 2\|10\|28 | 12\|20\|23 | 3\|12\|28 | F = 0.75 d.f. = 2.69 P = 0.477 |
| ALI.DAF | 2.25\|5.5\|20.75 | 1\|3\|17 | 2\|6\|17 | 1\|5.5\|17 | F = 0.17 d.f. = 2.69 P = 0.842 |
| PRESS.DAF | 0\|8.5\|21.75 | 0\|0\|19 | 0\|7\|14 | 0\|1\|18.25 | F = 0.2 d.f. = 2.69 P = 0.821 |
| PRESS2.DAF | 0\|8.5\|21.75 | 0\|1\|20 | 0\|7\|17 | 0\|1\|18.5 | F = 0.16 d.f. = 2.69 P = 0.855 |
| PRESS5.DAF | 0\|9\|23 | 0\|1\|20 | 0\|11\|18 | 0\|1.5\|20.25 | F = 0.22 d.f. = 2.69 P = 0.801 |
| PRESS15.DAF | 0\|13\|26 | 0\|3\|22 | 4\|14\|20 | 0\|5\|23 | F = 0.84 d.f. = 2.69 P = 0.435 |
| INO.DAF | 2\|13.5\|26 | 2\|8\|28 | 10\|19\|22 | 2\|12\|26.25 | F = 0.17 d.f. = 2.69 P = 0.845 |
| SIRS2.DAF | 0\|0\|4 | 0\|0\|3 | 0\|1\|2 | 0\|0\|3 | F = 0.83 d.f. = 2.69 P = 0.442 |
| SIRS3.DAF | 1.25\|3.5\|17 | 0\|2\|9 | 4\|7\|10 | 0\|3\|11.25 | F = 2.34 d.f. = 2.69 P = 0.104 |
| SIRS4.DAF | 2.5\|12\|25 | 1\|8\|22 | 8\|16\|20 | 2\|9\|22.25 | F = 1.33 d.f. = 2.69 P = 0.272 |
| STER.DAF | 0\|5\|25.0 | 1\|3\|19 | 1\|7\|15 | 0.75\|3.5\|19.25 | F = 0.01 d.f. = 2.69 P = 0.989 |
| CVS.DAF | 0\|4\|15.75 | 0\|0\|13 | 0\|3\|13 | 0\|0\|14 | F = 0.21 d.f. = 2.69 P = 0.814 |
| RESP.DAF | 0\|0\|10.75 | 0\|0\|8 | 0\|1\|5 | 0\|0\|8 | F = 0.04 d.f. = 2.69 P = 0.956 |
| PF300.DAF | 0\|0\|0.75 | 0\|0\|1 | 0\|0\|2 | 0\|0\|1 | F = 0.04 d.f. = 2.69 P = 0.962 |
| VENT.DAF | 0\|0\|10.5 | 0\|0\|8 | 0\|0\|2 | 0\|0\|8 | F = 0 32 d.f. = 2.69 P = 0.73 |
| CNS.DAF | 2.75\|12\|26.25 | 2\|7\|24 | 9\|13\|20 | 2\|10.5\|24.25 | F = 0.59 d.f. = 2.69 P = 0.556 |
| COAG.DAF | 2\|7\|27.75 | 1\|7\|26 | 4\|12\|20 | 1\|7.5\|26 | F = 0.25 d.f. = 2.69 P = 0.781 |
| INR.DAF | 1\|16.5\|28 | 1\|7\|26 | 6\|13\|21 | 1.75\|8\|26.25 | F = 0.42 d.f. = 2.69 P = 0.658 |
| ACRF.DAF | 0\|2\|25 | 0\|3\|24 | 5\|9\|14 | 0\|5\|20.25 | F = 0.45 d.f. = 2.69 P = 0.642 |
| ANYREN.DAF | 0\|2\|17.75 | 0\|3\|24 | 5\|9\|14 | 0\|5\|18.25 | F = 0.6 d.f. = 2.69 P = 0.549 |
| RENSUP.DAF | 1\|2.5\|26 | 1\|3\|28 | 2\|10\|17 | 1\|4.5\|27.25 | F = 0.14 d.f. = 2.69 P = 0.868 |
| ACHEP.DAF | 2.25\|8.5\|28 | 1\|3\|20 | 10\|14\|22 | 2\|7.5\|24 | F = 1.62 d.f. = 2.69 P = 0.204 |
| ANYHEP.DAF | 2.25\|8\|28 | 1\|3\|20 | 10\|14\|22 | 2\|7\|24 | F = 1.73 d.f. = 2.69 P = 0.186 |

N, number of subjects.

TABLE 3.36
A response association of arginine vasopressin receptor 1a AVPR1A rs1495027 in Caucasian
septic-shock control subjects.. For all variables besides 28-day survival, data is given as 25^{th}
percentile|median|75^{th} percentile. For 28-day survival, data is given as % (N survived/N total).

| | CC | CT | TT | Combined | Test |
|---|---|---|---|---|---|
| CONTROL | (N = 29) | (N = 37) | (N = 13) | (N = 79) | Statistic |
| SURVIVAL | 24% (7/29) | 35% (13/37) | 46% (6/13) | 33% (26/79) | Chisquare = 2.13 d.f. = 2 P = 0.345 |
| DAYS ALIVE | 2\|6\|21 | 3\|8\|28 | 4\|15\|28 | 3\|8\|28 | F = 0.77 d.f. = 2.76 P = 0.467 |
| ALI.DAF | 1\|3\|11 | 1\|5\|14 | 2\|7\|20 | 1\|5\|14.5 | F = 0.42 d.f. = 2.76 P = 0.661 |
| PRESS.DAF | 0\|3\|14 | 0\|4\|24 | 1\|9\|19 | 0\|4\|22 | F = 0.46 d.f. = 2.76 P = 0.633 |
| PRESS2.DAF | 0\|3\|14 | 0\|4\|24 | 2\|9\|19 | 0\|4\|22 | F = 0.48 d.f. = 2.76 P = 0.62 |
| PRESS5.DAF | 0\|3\|14 | 0\|4\|25 | 2\|9\|21 | 0\|4\|23 | F = 0.7 d.f. = 2.76 P = 0.501 |
| PRESS15.DAF | 0\|3\|18 | 1\|6\|26 | 2\|15\|26 | 0.5\|5\|26 | F = 1.04 d.f. = 2.76 P = 0.359 |
| INO.DAF | 1\|3\|20 | 3\|7\|28 | 2\|15\|28 | 2\|6\|28 | F = 1.15 d.f. = 2.76 P = 0.322 |
| SIRS2.DAF | 0\|0\|0 | 0\|0\|2 | 0\|0\|2 | 0\|0\|1 | F = 1.05 d.f. = 2.76 P = 0.355 |
| SIRS3.DAF | 1\|1\|5 | 0\|2\|8 | 2\|4\|9 | 0\|2\|6.5 | F = 0.94 d.f. = 2.76 P = 0.394 |
| SIRS4.DAF | 1\|6\|11 | 2\|5\|25 | 4\|10\|22 | 2\|6\|21.5 | F = 0.76 d.f. = 2.76 P = 0.471 |
| STER.DAF | 0\|2\|10 | 1\|5\|24 | 2\|5\|15 | 1\|5\|20 | F = 0.71 d.f. = 2.76 P = 0.495 |
| CVS.DAF | 0\|0\|13 | 0\|3\|18 | 0\|4\|19 | 0\|2\|18.5 | F = 0.45 d.f. = 2.76 P = 0.637 |
| RESP.DAF | 0\|1\|9 | 0\|2\|17 | 0\|1\|7 | 0\|1\|9.5 | F = 0.37 d.f. = 2.76 P = 0.694 |
| PF300.DAF | 0\|0\|0 | 0\|0\|2 | 0\|0\|0 | 0\|0\|1.5 | F = 1.42 d.f. = 2.76 P = 0.248 |
| VENT.DAF | 0\|0\|9 | 0\|0\|12 | 0\|0\|7 | 0\|0\|9.5 | F = 0.07 d.f. = 2.76 P = 0.93 |
| CNS.DAF | 1\|5\|18 | 1\|7\|26 | 4\|14\|25 | 1\|7\|25 | F = 0.34 d.f. = 2.76 P = 0.712 |
| COAG.DAF | 1\|5\|15 | 1\|6\|28 | 2\|15\|28 | 1\|6\|24.5 | F = 0.54 d.f. = 2.76 P = 0.583 |
| INR.DAF | 0\|3\|21 | 0\|5\|21 | 1\|10\|27 | 0\|4\|21.5 | F = 0.36 d.f. = 2.76 P = 0.701 |
| ACRF.DAF | 0\|3\|9 | 0\|6\|23 | 0\|10\|20 | 0\|4\|20 | F = 0.42 d.f. = 2.76 P = 0.658 |
| ANYREN.DAF | 0\|2\|9 | 0\|5\|23 | 0\|10\|20 | 0\|4\|19 | F = 0.28 d.f. = 2.76 P = 0.757 |
| RENSUP.DAF | 1\|2\|4 | 1\|7\|28 | 2\|5\|16 | 1\|4\|17 | F = 2.45 d.f. = 2.76 P = 0.0928 |
| ACHEP.DAF | 1\|5\|19 | 2\|7\|28 | 4\|15\|28 | 1\|6\|26 | F = 1.21 d.f. = 2.76 P = 0.303 |
| ANYHEP.DAF | 1\|5\|19 | 1\|6\|28 | 4\|15\|28 | 1\|6\|26 | F = 0.94 d.f. = 2.76 P = 0.397 |

N, number of subects.

TABLE 3.37
Difference in response association of arginine vasopressin receptor 1a (AVPR1A) rs1495027
between cases (vasopressin-treated group) (Treat) and controls (vasopressin untreated matched
control) (Cont) of Caucasian ICU subjects diagnosed with septic shock. For all variables besides
28-day survival, data is presented as medians. For 28-day survival, data is presented as % (N
survived/N total).

| | rs1495027 TT | | | rs1495027 CT | | | rs1495027 CC | | |
|---|---|---|---|---|---|---|---|---|---|
| | (N = 13) | (N = 13) | | (N = 45) | (N = 37) | | (N = 14) | (N = 29) | |
| | Treat | Cont | DELTA | Treat | Cont | DELTA | Treat | Cont | DELTA |
| SURVIVAL | 23% (3) | 46% (6) | −23% | 38% (17) | 35% (13) | 3% | 50% (7) | 24% (7) | 26% |
| DAYS ALIVE | 20 | 15 | 5 | 10 | 8 | 2 | 18.5 | 6 | 12.5 |
| ALI.DAF | 6 | 7 | −1 | 3 | 5 | −2 | 5.5 | 3 | 2.5 |
| PRESS.DAF | 7 | 9 | −2 | 0 | 4 | −4 | 8.5 | 3 | 5.5 |
| PRESS2.DAF | 7 | 9 | −2 | 1 | 4 | −3 | 8.5 | 3 | 5.5 |
| PRESS5.DAF | 11 | 9 | 2 | 1 | 4 | −3 | 9 | 3 | 6 |
| PRESS15.DAF | 14 | 15 | −1 | 3 | 6 | −3 | 13 | 3 | 10 |
| INO.DAF | 19 | 15 | 4 | 8 | 7 | 1 | 13.5 | 3 | 10.5 |
| SIRS2.DAF | 1 | 0 | 1 | 0 | 0 | 0 | 0 | 0 | 0 |
| SIRS3.DAF | 7 | 4 | 3 | 2 | 2 | 0 | 3.5 | 1 | 2.5 |
| SIRS4.DAF | 16 | 10 | 6 | 8 | 5 | 3 | 12 | 6 | 6 |
| STER.DAF | 7 | 5 | 2 | 3 | 5 | −2 | 5 | 2 | 3 |
| CVS.DAF | 3 | 4 | −1 | 0 | 3 | −3 | 4 | 0 | 4 |
| RESP.DAF | 1 | 1 | 0 | 0 | 2 | −2 | 0 | 1 | −1 |
| PF300.DAF | 0 | 0 | 0 | 0 | 0 | 0 | 0 | 0 | 0 |
| VENT.DAF | 0 | 0 | 0 | 0 | 0 | 0 | 0 | 0 | 0 |
| CNS.DAF | 13 | 14 | −1 | 7 | 7 | 0 | 12 | 5 | 7 |
| COAG.DAF | 12 | 15 | −3 | 7 | 6 | 1 | 7 | 5 | 2 |
| INR.DAF | 13 | 10 | 3 | 7 | 5 | 2 | 16.5 | 3 | 13.5 |
| ACRF.DAF | 9 | 10 | −1 | 3 | 6 | −3 | 2 | 3 | −1 |
| ANYREN.DAF | 9 | 10 | −1 | 3 | 5 | −2 | 2 | 2 | 0 |
| RENSUP.DAF | 10 | 5 | 5 | 3 | 7 | −4 | 2.5 | 2 | 0.5 |
| ACHEP.DAF | 14 | 15 | −1 | 3 | 7 | −4 | 8.5 | 5 | 3.5 |
| ANYHEP.DAF | 14 | 15 | −1 | 3 | 6 | −3 | 8 | 5 | 3 |

N, number of subjects.

A logistic regression approach was used to test for a statistically significant interaction between genotype and vasopressin use as predicted by 28-day survival TABLE 3.38 shows that there was a statistically significant interaction between AVPR1A rs1495027 genotype, vasopressin treatment and survival, confirming vasopressin treatment decreases 28-day survival in AVPR1A rs1495027 TT genotype subjects while vasopressin treatment increases 28-day survival in AVPR1A rs1495027 CC subjects compared to controls (P=0.04662). Following adjustment for age, admission APACHE II score, gender, medical, surgical diagnosis and days alive and free of 3 of 4 systematic inflammatory response syndrome (SIRS) criteria, there was still a statistically significant interaction of the AVPR1A rs1495027 genotype and treatment with vasopressin (P=0.0339).

TABLE 3.38
Interaction between genotype and vasopressin use vs. no vasopressin
(Controls) and CC or CT genotype vs. TT genotype of arginine
vasopressin receptor 1a (AVPR1A) rs1495027 on 28-day survival.

| | Estimate | Std. Error | z value | Pr(>\|z\|) |
|---|---|---|---|---|
| Vasopressin vs. controls + | 2.195 | 1.1031 | 1.99 | 0.04662 |
| genotype interaction | | | | |
| Vasopressin vs. controls + | 2.6035 | 1.2271 | 2.122 | 0.03387 |
| genotype interaction − | | | | |
| Adjusted | | | | |

### Example 1 Summary

Genotyping of SNPs LNPEP rs18059, LNPEP rs27711, LNPEP rs10051637, AVP rs1410713, AVP rs857240, AVP rs857242, and AVPR1A rs1495027 in subjects with septic shock can predict response to administration of vasopressin as measured by 28-day survival and/or DAF of organ dysfunction. Subjects with genotypes including LNPEP rs18059 CC, LNPEP rs27711 AA, LNPEP rs10051637 GG, AVP rs1410713 CC, AVP rs857240 CT, AVP rs857242 AC and AVPR1A rs1495027 TT should not be administered a vasopressin receptor agonist as this could potentially decrease survival and increase risk of organ dysfunction. In contrast, subjects with LNPEP rs18059 TT, LNPEP rs27711 GG, LNPEP rs10051637 AA, AVP rs1410713 AA and rs1410713 AC, AVP rs857240 CC. AVP rs857242 CC and AVPR1A rs1495027 CC genotypes should be administered a vasopressin receptor agonist as such treatment has the potential to increase survival and decrease risk of organ dysfunction.

### Example 2

### Risk of Death and Organ Dysfunction

### Methods

### Cohort Selection

To investigate whether genotype predicts risk of death and organ dysfunction, selected subsets of the ICU cohort were used for this study. All patients who were treated with vasopressin for septic shock were excluded. The four study groups were: ICU Caucasians with SIRS upon admission (n=874), ICU Caucasians with sepsis upon admission (n=690). ICU Caucasians with septic shock upon admission (n=440) and ICU Asians with SIRS upon admission (n=108).

### Data Analysis

All data analysis was carried out using statistical packages available in R(R Core Development Group, 2005-R Development Core Team (www.R-project.org). R: A language and environment for statistical computing. Vienna, Austria. 2005). Chi-square and Kruskal-Wallis (KW) test statistics were used in conjunction with Cox proportional hazards (CPH) regression to identify significant SNP-phenotype associations, as well as to identify baseline characteristics (age, gender, admitting APACHE II score, and medical vs. surgical admitting diagnosis) requiring post-hoc, multivariate adjustment. Genetically heterogenous populations were subsetted prior to analysis to avoid confounding from potential population stratification.

### Results

### 2.1 Leucyl/Cystinyl Aminopeptidase (LNPEP)

2.1.1 LNPEP rs18059

### 2.1.1.1 Systematic Inflammatory Response Syndrome—Caucasians

TABLE 4.1 gives the baseline characteristics of 710 Caucasian SIRS subjects who were successfully genotyped (CC vs. CT/TT) at LNPEP rs18059. No significant differences were detected between the two genotype groups on admission to the ICU.

TABLE 4.1
Baseline characteristics of a cohort of Caucasian Subjects with systematic inflammatory response
syndrome by genotype of leucyl/cystinyl aminopeptidase (LNPEP) rs18059 (CC vs. CT/TT). For
age and APACHE II score, data is given as 25^{th} percentile/median/75^{th} percentile. For all other
variables, data is given as % (N survived/N total).

| | CC | CT/TT | Combined | Test |
|---|---|---|---|---|
| | (N = 155) | (N = 555) | (N = 710) | Statistic |
| AGE | 44.5/58/70 | 45/59/71 | 46/59/71 | F = 0.96 d.f. = 1.708 P = 0.327 |
| GENDER | 63% (97/155) | 61% (336/555) | 61% (433/710) | X\hat{ }2 = 0.21 d.f. = 1 P = 0.645 |
| APACHE II | 15/20/26 | 16/22/27 | 16/21.5/27 | F = 2.52 d.f. = 1.708 P = 0.113 |
| SURGICAL | 20% (31/155) | 23% (130/555) | 23% (161/710) | X\hat{ }2 = 0.81 d.f. = 1 P = 0.368 |
| SEP.ADMIT | 81% (125/155) | 78% (435/555) | 79% (560/710) | X\hat{ }2 = 0.37 d.f. = 1 P = 0.541 |
| SEP.ANY | 83% (129/155) | 80% (442/555) | 80% (571/710) | X\hat{ }2 = 0.99 d.f. = 1 P = 0.32 |
| SS.ADMIT | 52% (81/155) | 51% (285/555) | 52% (366/710) | X\hat{ }2 = 0.04 d.f. = 1 P = 0.842 |
| SS.ANY | 55% (85/155) | 55% (306/555) | 55% (391/710) | X\hat{ }2 = 0 d.f. = 1 P = 0.948 |

N, number of subjects.

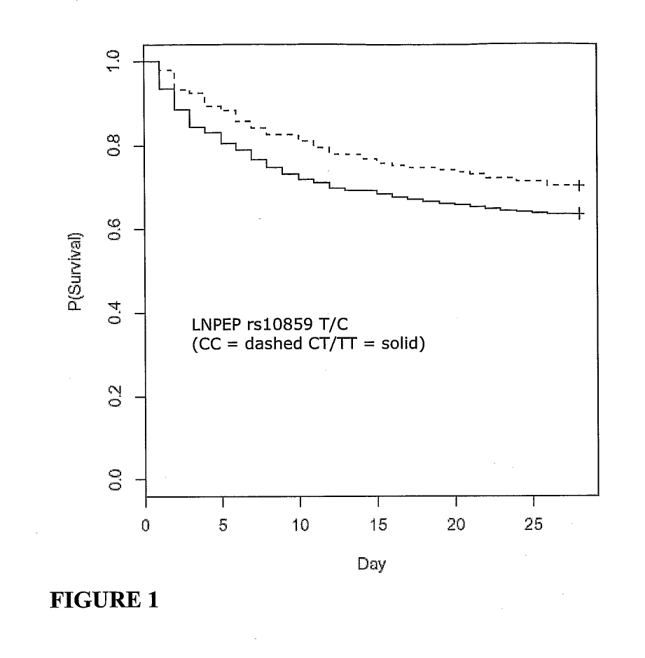

FIG. 1 and TABLE 4.2 summarize important SNP-phenotype associations. Subjects with LNPEP rs18059 CC genotype showed a significantly greater survival (P=0.0331) and had significantly more days alive (P=0.0144) and days alive and free of vasopressors (P=0.0088), days alive and free of vasopressors at doses of more than 2 ug/min (P=0.0101). 5 ug/min (P=0.037) and 15 ug/min (P=0.0157), inotropes (P=0.0252), coagulation dysfunction (P=0.0030), any renal dysfunction (P=0.0088), renal support (P=0.0145), acute hepatic dysfunction (P=0.0335) and any hepatic dysfunction (P=0.0456). Subjects who carried the LNPEP rs18059 CC genotype also showed a strong trend for more days alive and free of neurological dysfunction (P=0.071). These findings indicate that these patients who have who carry the LNPEP rs18059 CC genotype at LNPEP rs18059 CC have less need of inotrope and vasopressor therapy and have a lower risk of organ dysfunction (coagulation, renal, hepatic and neurological).

TABLE 4.2
Days alive and free of organ dysfunction (DAF) by allele of leucyl/cystinyl
aminopeptidase (LNPEP) rs18059 (CC vs. CT/TT) in a cohort of Caucasian
subjects with systematic inflammatory response syndrome. For all variables
besides 28-day survival, data is given as 25^{th} percentile/median/75^{th} percentile.
For 28-day survival, data is given as % (N survived/N total).

| | CC | CT/TT | Combined | Test |
|---|---|---|---|---|
| | (N = 155) | (N = 555) | (N = 710) | Statistic |
| SURVIVAL | 75% (117/155) | 66% (369/555) | 68% (486/710) | X\hat{ }2 = 4.54 d.f. = 1 P = 0.0331 |
| DA | 28/28/28 | 10/28/28 | 12/28/28 | F = 6.02 d.f. = 1.708 P = 0.0144 |
| PRESS.DAF | 17.5/27/28 | 7/25/28 | 9/26/28 | F = 6.9 d.f. = 1.708 P = 0.0088 |
| PRESS2.DAF | 17.5/27/28 | 7.5/26/28 | 10/26/28 | F = 6.64 d.f. = 1.708 P = 0.0101 |
| PRESS5.DAF | 18.5/27/28 | 8/26/28 | 10/26/28 | F = 8.49 d.f. = 1.708 P = 0.00369 |
| PRESS15.DAF | 23.5/28/28 | 9/28/28 | 12/28/28 | F = 5.86 d.f. = 1.708 P = 0.0157 |
| INO.DAF | 24/28/28 | 9/28/28 | 11.3/28/28 | F = 5.03 d.f. = 1.708 P = 0.0252 |
| CNS.DAF | 14/27/28 | 7/26/28 | 7.25/27/28 | F = 3.27 d.f. = 1.708 P = 0.071 |
| COAG.DAF | 20/28/28 | 7/28/28 | 8.25/28/28 | F = 8.87 d.f. = 1.708 P = 0.00299 |
| INR.DAF | 14/28/28 | 5/27/28 | 7/27/28 | F = 3.51 d.f. = 1.708 P = 0.0615 |
| ANYREN.DAF | 9/28/28 | 2/22/28 | 3/25/28 | F = 6.9 d.f. = 1.708 P = 0.00882 |
| RENSUP.DAF | 14/28/28 | 4/28/28 | 5/28/28 | F = 6 d.f. = 1.708 P = 0.0145 |
| ACHEP.DAF | 17/28/28 | 7/28/28 | 8/28/28 | F = 4.54 d.f. = 1.708 P = 0.0335 |
| ANYHEP.DAF | 15.5/28/28 | 6/28/28 | 7/28/28 | F = 4.01 d.f. = 1.708 P = 0.0456 |

N, number of subjects.

### 2.1.1.2 Sepsis—Caucasians

TABLE 4.3 gives the baseline characteristics (age, gender, APACHE II score, medical vs. surgical diagnosis, septic shock upon admission and septic shock anytime) of 561 Caucasian sepsis subjects who were successfully genotyped (CC vs. CT/TT) at LNPEP rs18059. No significant differences were detected between the two genotype groups on admission to the ICU.

TABLE 4.3
Baseline characteristics of a cohort of Caucasian Subjects with sepsis by allele of
leucyl/cystinyl aminopeptidase (LNPEP) rs18059 (CC vs. CT/TT). For age and
APACHE II score, data is given as 25^{th} percentile/median/75^{th} percentile.
For all other variables, data is given as % (N survived/N total).

| | CC | CT/TT | Combined | Test |
|---|---|---|---|---|
| | (N = 126) | (N = 435) | (N = 561) | Statistic |
| AGE | 46/58/70.8 | 45/59/71.5 | 47/59/72 | F = 0.45 d.f. = 1.559 P = 0.501 |
| GENDER | 65% (82/126) | 62% (270/435) | 63% (352/561) | X\hat{ }2 = 0.38 d.f. = 1 P = 0.538 |
| APACHE II | 16/22/27 | 17/23/28 | 17/22/28 | F = 1.95 d.f. = 1.559 P = 0.163 |
| SURGICAL | 21% (26/126) | 23% (100/435) | 22% (126/561) | X\hat{ }2 = 0.31 d.f. = 1 P = 0.577 |
| SS.ADMIT | 64% (81/126) | 66% (285/435) | 65% (366/561) | X\hat{ }2 = 0.07 d.f. = 1 P = 0.798 |
| SS.ANY | 66% (83/126) | 70% (303/435) | 69% (386/561) | X\hat{ }2 = 0.65 d.f. = 1 P = 0.42 |

N, number of subjects.

TABLE 4.4 summarizes important SNP-phenotype associations. Subjects with the LNPEP rs18059 CC genotype showed significantly more days alive and free of vasopressors (P=0.0377), days alive and free of vasopressors at doses of more than 2 ug/min (P=0.0424) and 5 ug/min (P=0.0194) and coagulation dysfunction (P=0.0359). Subjects who carried the LNPEP rs18059 CC genotype also showed a strong trend for more days alive and free of renal support (P=0.07). These findings indicate that Caucasian sepsis subjects who carry the LNPEP rs18059 CC genotype have less need of vasopressor therapy and have a lower risk of organ dysfunction (coagulation and renal).

TABLE 4.4
Days alive and free of organ dysfunction (DAF) by allele of leucyl/cystinyl
aminopeptidase (LNPEP) rs18059 (CC vs. CT/TT) in a cohort of Caucasian
subjects with sepsis. Data is given as 25^{th} percentile/median/75^{th} percentile.

| | | CT/TT | Combined | Test |
|---|---|---|---|---|
| | CC (N = 126) | (N = 435) | (N = 561) | Statistic |
| PRESS.DAF | 15/26/28 | 8/25/28 | 10/25/28 | F = 4.34 d.f. = 1.559 P = 0.0377 |
| PRESS2.DAF | 15/26/28 | 8.5/25/28 | 10/25/28 | F = 4.14 d.f. = 1.559 P = 0.0424 |
| PRESS5.DAF | 17.3/27/28 | 9/25/28 | 11/26/28 | F = 5.5 d.f. = 1.559 P = 0.0194 |
| COAG.DAF | 20/28/28 | 9/28/28 | 0/28/28 | F = 6.06 d.f. = 1.559 P = 0.0142 |
| RENSUP.DAF | 11.3/28/28 | 5/28/2 | 6/28/28 | F = 3.29 d.f. = 1.559 P = 0.07 |

N, number of subjects.

### 2.1.1.3 Septic Shock—Caucasians

TABLE 4.5 gives the baseline characteristics (age, gender, APACHE II score and medical vs. surgical diagnosis) of 366 Caucasian septic shock subjects who were successfully genotyped (CC vs. CT/TT) at LNPEP rs18059. No significant differences were detected between the two genotype groups on admission to the ICU.

TABLE 4.5
Baseline characteristics of a cohort of Caucasian Subjects with septic shock by allele of
leucyl/cystinyl aminopeptidase (LNPEP) rs18059 (CC vs. CT/TT). For age and APACHE II
score, data is given as 25^{th} percentile/median/75^{th} percentile. For all other variables,
data is given as % (N survived/N total).

| | CC | CT/TT | Combined | Test |
|---|---|---|---|---|
| | (N = 81) | (N = 285) | (N = 366) | Statistic |
| AGE | 47/59/71 | 48/63/73 | 48/62/73 | F = 1.91 d.f. = 1.364 P = 0.168 |
| GENDER | 64% (52/81) | 60% (172/285) | 61% (224/366) | X\hat{ }2 = 0.39 d.f. = 1 P = 0.531 |
| APACHEII | 17/24/29 | 20/25/30 | 19/24/30 | F = 1.81 d.f. = 1.364 P = 0.180 |
| SURGICAL | 21% (17/81) | 26% (74/285) | 25% (91/366) | X\hat{ }2 = 0.84 d.f. = 1 P = 0.360 |

N, number of subjects.

TABLE 4.6 summarizes important SNP-phenotype associations. Subjects with the LNPEP rs18059 CC genotype showed a strong trend for greater survival (P=0.0862) and significantly more days alive (P=0.0353) and days alive and free of vasopressors (P=0.0404), days alive and free of vasopressors at doses of more than 2 ug/min (P=0.0372), 5 ug/min (P=0.0132) and 15 ug/min (P=0.0373), coagulation dysfunction (P=0.0079), any renal dysfunction (P=0.0394) and renal support (P=0.0364). LNPEP rs18059 CC individuals also showed a strong trend for more days alive and free of inotropes (P=0.0646) and acute renal dysfunction (P=0.0593). These findings indicate that Caucasian septic shock subjects who carry the CC genotype at LNPEP rs18059 have less need of inotrope and vasopressor therapy and are have a lower risk of organ dysfunction (coagulation and renal).

TABLE 4.6
Days alive and free of organ dysfunction (DAF) by allele of leucyl/cystinyl
aminopeptidase (LNPEP) rs18059 (CC vs. CT/TT) in a cohort of Caucasian
subjects with septic shock. For all variables besides 28-day survival, data is
given as 25^{th} percentile/median/75^{th} percentile. For 28-day survival,
data is given as % (N survived/N total).

| | CC | CT/TT | Combined | Test |
|---|---|---|---|---|
| | (N = 81) | (N = 285) | (N = 366) | Statistic |
| SURVIVAL | 69% (56/81) | 59% (167/285) | 61% (223/366) | X\hat{ }2 = 2.94 d.f. = 1 P = 0.0862 |
| DA | 22/28/28 | 8/28/28 | 9/28/28 | F = 4.46 d.f. = 1.364 P = 0.0353 |
| PRESS.DAF | 11/24/27 | 4/21/26 | 5.75/23/26 | F = 4.23 d.f. = 1.364 P = 0.0404 |
| PRESS2.DAF | 11/24/27 | 4/22/26 | 5.75/23/26 | F = 4.37 d.f. = 1.364 P = 0.0372 |
| PRESS5.DAF | 13/25/27 | 5/23/27 | 6/24/27 | F = 6.2 d.f. = 1.364 P = 0.0132 |
| PRESS15.DAF | 17/27/28 | 6/26/28 | 8/26/28 | F = 4.37 d.f. = 1.364 P = 0.0373 |
| INO.DAF | 18/28/28 | 6/26/28 | 7/28/28 | F = 3.44 d.f. = 1.364 P = 0.0646 |
| COAG.DAF | 17/28/28 | 5/24/28 | 6/25/28 | F = 7.14 d.f. = 1.364 P = 0.0079 |
| INR.DAF | 12/25/28 | 4/22/28 | 5/24/28 | F = 2.81 d.f. = 1.364 P = 0.0944 |
| ACRF.DAF | 10/27/28 | 3/20/28 | 3/22/28 | F = 3.58 d.f. = 1.364 P = 0.0593 |
| ANYREN.DAF | 9/26/28 | 2/18/28 | 2.75/19.50/28 | F = 4.27 d.f. = 1.364 P = 0.0394 |
| RENSUP.DAF | 10/28/28 | 3/23/28 | 4/25/28 | F = 4.41 d.f. = 1.364 P = 0.0364 |

N, number of subjects.

2.1.2 LNPEP rs27711

### 2.1.2.2 Systematic Inflammatory Response Syndrome—Caucasians

TABLE 4.7 summarizes the baseline characteristics (age, gender, APACHE II score, medical vs. surgical diagnosis, sepsis upon admission, sepsis anytime, septic shock upon admission and septic shock anytime) of 717 Caucasian systematic inflammatory response syndrome subjects who were successfully genotyped (AA vs. GG/AG) at LNPEP rs27711. No significant differences were detected between the two genotype groups on admission to the ICU.

TABLE 4.7
Baseline characteristics of a cohort of Caucasian Subjects with systematic
inflammatory response syndrome by genotype of leucyl/cystinyl aminopeptidase
(LNPEP) rs27711 (GG/AG vs. AA). For age and APACHE II score, data is given
as 25^{th} percentile/median/75^{th} percentile. For all other variables, data is given
as % (N survived/N total).

| | AA | GG/AG | Combined | Test |
|---|---|---|---|---|
| | (N = 98) | (N = 619) | (N = 717) | Statistic |
| AGE | 43.5/ | 45/59/70.5 | 46/59/71 | F = 1.1 d.f. = 1.715 P = 0.294 |
| | 57/71 | | | |
| GENDER | 60% | 62% (382/619) | 62% | X\hat{ }2 = 0.08 d.f. = 1 P = 0.776 |
| | (59/98) | | (441/717) | |
| APACHEII | 15/20/ | 16/22/27 | 16/21.5/ | F = 1.42 d.f. = 1.715 P = 0.234 |
| | 27 | | 27 | |
| SURGICAL | 19% | 23% (141/619) | 22% | X\hat{ }2 = 0.56 d.f. = 1 P = 0.454 |
| | (19/98) | | (160/717) | |
| SEP.ADMIT | 79% | 79% (487/619) | 79% | X\hat{ }2 = 0 d.f. = 1 P = 0.981 |
| | (77/98) | | (564/717) | |
| SEP.ANY | 81% | 80% (497/619) | 80% | X\hat{ }2 = 0.01 d.f. = 1 P = 0.94 |
| | (79/98) | | (576/717) | |
| SS.ADMIT | 52% | 51% (317/619) | 51% | X\hat{ }2 = 0.02 d.f. = 1 P = 0.879 |
| | (51/98) | | (368/717) | |
| SS.ANY | 52% | 55% (342/619) | 55% | X\hat{ }2 = 0.35 d.f. = 1 P = 0.553 |
| | (51/98) | | (393/717) | |

N, number of subjects.

TABLE 4.8 summarizes important SNP-phenotype associations. Subjects with the LNPEP rs27711 AA genotype showed significantly more days alive and free of vasopressors (P=0.0330), days alive and free of vasopressors at doses of more than 2 ug/min (P=0.0362), 5 ug/min (P=0.0222) and 15 ug/min (P=0.0961). Subjects with the LNPEP rs27711 AA genotype also had a strong trend for more days alive and free of steroids (P=0.0871). These findings indicate that Caucasian subjects who have SIRS and have the AA genotype at LNPEP rs27711 have less need for vasopressor therapy and steroid therapy.

TABLE 4.8
Days alive and free of organ dysfunction (DAF) by allele of leucyl/cystinyl
aminopeptidase (LNPEP) rs27711 (GG/AG vs. AA) in a cohort of Caucasian
subjects with systematic inflammatory response syndrome. Data is given as
25^{th} percentile/median/75^{th} percentile.

| | | GG/AG | Combined | Test |
|---|---|---|---|---|
| | AA (N = 98) | (N = 619) | (N = 717) | Statistic |
| PRESS.DAF | 15/27/ | 9/26/28 | 9/26/28 | F = 4.56 d.f. = 1.715 P = 0.0330 |
| | 28 | | | |
| PRESS2.DAF | 15/27/ | 9/26/28 | 10/26/28 | F = 4.41 d.f. = 1.715 P = 0.0362 |
| | 28 | | | |
| PRESS5.DAF | 17/28/ | 10/26/28 | 10/26/28 | F = 5.25 d.f. = 1.715 P = 0.0222 |
| | 28 | | | |
| PRESS15.DAF | 20.5/28/ | 11/28/28 | 12/28/28 | F = 2.78 d.f. = 1.715 P = 0.0961 |
| | 28 | | | |
| STER.DAF | 6/26.5/ | 2/22/28 | 2/23/28 | F = 2.93 d.f. = 1.715 P = 0.0871 |
| | 28 | | | |

N, number of subjects.

2.1.3 LNPEP rs10051637

### 2.1.3.1 Systematic Inflammatory Response Syndrome—Caucasians

TABLE 4.9 summarizes the baseline characteristics (age, gender, APACHE II score, medical vs. surgical diagnosis, sepsis upon admission, sepsis anytime, septic shock upon admission and septic shock anytime) of 710 Caucasian SIRS subjects who were successfully genotyped (AA vs. AG/GG) at LNPEP rs10051637. No significant baseline differences were detected between the two genotype groups on admission to the ICU although the AG/GG group is more likely to be diagnosed with sepsis throughout an ICU stay.

TABLE 4.9
Baseline characteristics of a cohort of Caucasian Subjects with systematic
inflammatory response syndrome by genotype of leucyl/cystinyl aminopeptidase
(LNPEP) rs10051637 (AA vs. AG/GG). For age and APACHE II score, data is
given as 25^{th} percentile/median/75^{th} percentile. For all other variables,
data is given as % (N survived/N total).

| | AA | AG/GG | Combined | Test |
|---|---|---|---|---|
| | (N = 236) | (N = 474) | (N = 710) | Statistic |
| AGE | 44/61/72 | 45.3/58/ | 46/59/ | F = 1.06 d.f. = 1.708 P = 0.304 |
| | | 70 | 71 | |
| GENDER | 60% (142/236) | 63% | 62% | X\hat{ }2 = 0.41 d.f. = 1 P = 0.52 |
| | | (297/474) | (439/710) | |
| APACHEII | 17/22/27 | 15/22/27 | 16/21.5/ | F = 0.2 d.f. = 1.708 P = 0.657 |
| | | | 27 | |
| SURGICAL | 21% (49/236) | 24% | 23% | X\hat{ }2 = 0.85 d.f. = 1 P = 0.357 |
| | | (113/474) | (162/710) | |
| SEP.ADMIT | 75% (177/236) | 80% | 78% | X\hat{ }2 = 2.08 d.f. = 1 P = 0.149 |
| | | (378/474) | (555/710) | |
| SEP.ANY | 76% (179/236) | 82% | 80% | X\hat{ }2 = 3.81 d.f. = 1 P = 0.051 |
| | | (389/474) | (568/710) | |
| SS.ADMIT | 48% (114/236) | 52% | 51% | X\hat{ }2 = 0.91 d.f. = 1 P = 0.339 |
| | | (247/474) | (361/710) | |
| SS.ANY | 51% (121/236) | 56% | 55% | X\hat{ }2 = 1.49 d.f. = 1 P = 0.222 |
| | | (266/474) | (387/710) | |

N, number of subjects.

TABLE 4.10 summarizes important SNP-phenotype associations. Subjects with the LNPEP rs10051637 AG or GG genotype showed significantly more days alive and free of inotropes (P=0.0357) and 2 of 4 SIRS criteria (P=0.0226). These findings indicate that Caucasian subjects who have SIRS who carry either the AG or GG genotype at LNPEP rs10051637 have less need of inotrope therapy and less SIRS.

TABLE 4.10
Days alive and free of organ dysfunction (DAF) by allele of leucyl/cystinyl
aminopeptidase (LNPEP) rs10051637 (AA vs. AG/GG) in a cohort of
Caucasian subjects with systematic inflammatory response syndrome.
Data is given as 25^{th} percentile/median/75^{th} percentile.

| | | AG/ | | |
|---|---|---|---|---|
| | | GG | Combined | Test |
| | AA (N = 236) | (N = 474) | (N = 710) | Statistic |
| INO.DAF | 7/28/28 | 15/28/ | 11.3/28/ | F = 4.43 d.f. = 1.708 P = 0.0357 |
| | | 28 | 28 | |
| MSIRS2.DAF | 0/2/20 | 0/6/21 | 0/5/21 | F = 5.22 d.f. = 1.708 P = 0.0226 |
| CSIRS2.DAF | 0/3/20 | 0/5/20 | 0/5/20 | F = 3.23 d.f. = 1.708 P = 0.0726 |

N, number of subjects.

2.1.4 LNPEP rs38041

### 2.1.4.1 Systematic Inflammatory Response Syndrome—Caucasians

TABLE 4.11 summarizes the baseline characteristics (age, gender, APACHE II score, medical vs. surgical diagnosis, sepsis upon admission, sepsis anytime, septic shock upon admission and septic shock anytime) of 717 Caucasian SIRS subjects who were successfully genotyped (AA vs. GG/AG) at LNPEP rs38041. No significant differences were detected between the two genotype groups on admission to the ICU.

TABLE 4.11
Baseline characteristics of a cohort of Caucasian Subjects with systematic
inflammatory response syndrome by genotype of leucyl/cystinyl aminopeptidase
(LNPEP) rs38041 (AA vs. GG/AG). For age and APACHE II score, data is given
as 25^{th} percentile/median/75^{th} percentile. For all other variables, data is
given as % (N survived/N total).

| | AA | GG/AG | Combined | Test |
|---|---|---|---|---|
| | (N = 143) | (N = 574) | (N = 717) | Statistic |
| AGE | 45.5/56/ | 45/59/71 | 46/59/71 | F = 1.15 d.f. = 1.715 P = 0.283 |
| | 70.5 | | | |
| GENDER | 59% | 62% | 62% (441/717) | X\hat{ }2 = 0.32 d.f. = 1 P = 0.57 |
| | (85/143) | (356/574) | | |
| APACHEII | 15/21/27 | 16/22/27 | 16/21.5/27 | F = 0.84 d.f. = 1.715 P = 0.361 |
| SURGICAL | 24% | 22% | 23% (163/717) | X\hat{ }2 = 0.31 d.f. = 1 P = 0.579 |
| | (35/143) | (128/574) | | |
| SEP.ADMIT | 82% | 78% | 78% (562/717) | X\hat{ }2 = 1.24 d.f. = 1 P = 0.265 |
| | (117/143) | (445/574) | | |
| SEP.ANY | 83% | 79% | 80% (575/717) | X\hat{ }2 = 1.03 d.f. = 1 P = 0.311 |
| | (119/143) | (456/574) | | |
| SS.ADMIT | 52% | 50% | 51% (364/717) | X\hat{ }2 = 0.2 d.f. = 1 P = 0.653 |
| | (75/143) | (289/574) | | |
| SS.ANY | 55% | 54% | 54% (390/717) | X\hat{ }2 = 0 d.f. = 1 P = 0.967 |
| | (78/143) | (312/574) | | |

N, number of subjects.

TABLE 4.12 summarizes important SNP-phenotype associations for LNPEP rs38041. Subjects with the LNPEP rs38041 AA genotype showed significantly more days alive and free of vasopressors at doses of more than 5 ug/min (0.0278) and 15 ug/min (0.0384) and any renal dysfunction (P=0.0475). Subjects with the LNPEP rs38041 AA genotype also showed a strong trend for more days alive and free of vasopressors (P=0.067) and days alive and free of vasopressors at a dose of more than 2 ug/min (0.0751). These findings indicate that Caucasian subjects who have SIRS and have the AA genotype at LNPEP rs38041 have less need of vasopressor therapy and are a lower risk of organ dysfunction (renal).

TABLE 4.12
Days alive and free of organ dysfunction (DAF) by allele of
leucyl/cystinyl aminopeptidase (LNPEP) rs38041 (GG/AG vs. AA)
in a cohort of Caucasian subjects with systematic inflammatory
response syndrome. Data is given as 25^{th} percentile/
median/75^{th} percentile.

| | AA | GG/AG | | |
|---|---|---|---|---|
| | (N = | (N = | Combined | Test |
| | 143) | 574) | (N = 717) | Statistic |
| PRESS.DAF | 15/26/ | 8/26/ | 9/26/28 | F = 3.37 d.f. = 1.715 |
| | 28 | 28 | | P = 0.067 |
| PRESS2.DAF | 15/26/ | 8.25/26/ | 10/26/28 | F = 3.18 d.f. = 1.715 |
| | 28 | 28 | | P = 0.0751 |
| PRESS5.DAF | 17/27/ | 9/26/ | 10/26/28 | F = 4.86 d.f. = 1.715 |
| | 28 | 28 | | P = 0.0278 |
| PRESS15.DAF | 21/28/ | 10.3/28/ | 12/28/28 | F = 4.3 d.f. = 1.715 |
| | 28 | 28 | | P = 0.0384 |
| ANYREN.DAF | 9/28/ | 2/24/ | 3/25/28 | F = 3.94 d.f. = 1.715 |
| | 28 | 28 | | P = 0.0475 |

N, number of subjects.

### Arginine Vasopressin (AVP)

2.2.1 AVP rs1410713

2.2.1.1 Systematic Inflammatory Response Syndrome—Caucasians

TABLE 4.13 summarizes the baseline characteristics (age, gender, APACHE II score, medical vs. surgical diagnosis, sepsis upon admission, sepsis anytime, septic shock upon admission and septic shock anytime) of 717 Caucasian SIRS subjects who were successfully genotyped at AVP rs1410713. No significant differences were detected between the genotype groups on admission to the ICU.

TABLE 4.13
Baseline characteristics of a cohort of Caucasian Subjects with systematic inflammatory
response syndrome by genotype of Arginine Vasopressin (AVP) rs1410713
(AA vs. CC/AC). For age and APACHE II score, data is given as 25^{th}
percentile/median/75^{th} percentile. For all other
variables, data is given as % (N survived/N total).

| | AA | CC/AC | Combined | Test |
|---|---|---|---|---|
| | (N = 49) | (N = 668) | (N = 717) | Statistic |
| AGE | 48/59/74 | 45/59/70 | 46/59/71 | F = 1.01 d.f. = 1.715 P = 0.315 |
| GENDER | 51% (25/49) | 62% (416/668) | 62% (441/717) | X\hat{ }2 = 2.44 d.f. = 1 P = 0.118 |
| APACHEII | 16/23/28 | 16/22/27 | 16/21.5/27 | F = 0.25 d.f. = 1.715 P = 0.617 |
| SURGICAL | 18% (9/49) | 23% (155/668) | 23% (164/717) | X\hat{ }2 = 0.61 d.f. = 1 P = 0.437 |
| SEP.ADMIT | 82% (40/49) | 78% (523/668) | 79% (563/717) | X\hat{ }2 = 0.3 d.f. = 1 P = 0.583 |
| SEP.ANY | 82% (40/49) | 80% (536/668) | 80% (576/717) | X\hat{ }2 = 0.06 d.f. = 1 P = 0.813 |
| SS.ADMIT | 47% (23/49) | 51% (343/668) | 51% (366/717) | X\hat{ }2 = 0.36 d.f. = 1 P = 0.551 |
| SS.ANY | 49% (24/49) | 55% (367/668) | 55% (391/717) | X\hat{ }2 = 0.65 d.f. = 1 P = 0.419 |

N, number of subjects.

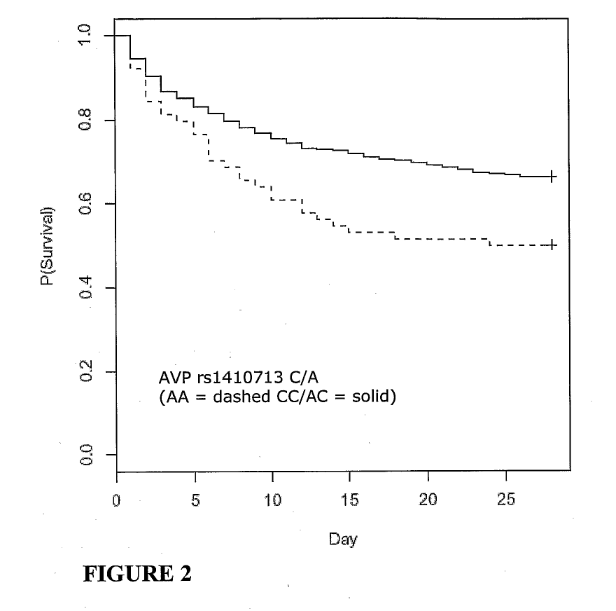

FIG. 2 and TABLE 4.14 summarize important SNP-phenotype associations for AVP rs1410713. Subjects in the AVP rs1410713 CC/AC genotype group had significantly increased survival (P=0.0140), significantly more days alive (P=0.0149) and significantly more days alive and free of neurological dysfunction (P=0.0482), coagulation dysfunction (P=0.0167), INR>1.5 (P=0.0108), acute renal dysfunction (P=0.0414), acute hepatic dysfunction (P=0.0218) and any hepatic dysfunction (P=0.0175). The AVP rs1410713 AA group also showed a strong trend for fewer days alive and free of inotropes (P=0.0709). These findings indicate that Caucasian subjects with SIRS and either the AVP rs1410713 CC or AC genotype have a lower risk of organ dysfunction (neurological, coagulation, renal and hepatic).

TABLE 4.14
Days alive and free of organ dysfunction (DAF) by genotype of Arginine Vasopressin (AVP)
rs1410713 (AA vs. CC/AC) in a cohort of Caucasian subjects with systematic inflammatory
response syndrome. For all variables besides 28-day survival, data is given as 25^{th} percentile/
median/75^{th} percentile. For 28-day survival, data is given as % (N survived/N total).

| | AA | CC/AC | Combined | Test |
|---|---|---|---|---|
| | (N = 49) | (N = 668) | (N = 717) | Statistic |
| SURVIVAL | 53% (26/49) | 70% (467/668) | 69% (493/717) | X\hat{ }2 = 6.03 d.f. = 1 P = 0.0140 |
| DA | 6/28/28 | 15/28/28 | 12/28/28 | F = 5.96 d.f. = 1.715 P = 0.0149 |
| INO.DAF | 6/28/28 | 13.8/28/28 | 11.3/28/28 | F = 3.27 d.f. = 1.715 P = 0.0709 |
| CNS.DAF | 2/22/28 | 8.75/27/28 | 7.25/27/28 | F = 3.91 d.f. = 1.715 P = 0.0482 |
| COAG.DAF | 3/20/28 | 10/28/28 | 8.25/28/28 | F = 5.75 d.f. = 1.715 P = 0.0167 |
| INR.DAF | 2/15/28 | 7/27/28 | 7/27/28 | F = 6.53 d.f. = 1.715 P = 0.0108 |
| ACRF.DAF | 2/16/28 | 5.75/27/28 | 5/27/28 | F = 4.18 d.f. = 1.715 P = 0.0414 |
| ACHEP.DAF | 6/22/28 | 8.75/28/28 | 8/28/28 | F = 5.28 d.f. = 1.715 P = 0.0218 |
| ANYHEP.DAF | 4/20/28 | 7/28/28 | 7/28/28 | F = 5.67 d.f. = 1.715 P = 0.0175 |

N, number of subjects.

2.2.1.2. Sepsis—Caucasians

TABLE 4.15 summarizes the baseline characteristics (age, gender, APACHE II score, medical vs. surgical diagnosis and shock upon admission and septic shock anytime) of 564 Caucasian sepsis subjects who were successfully genotyped at AVP rs1410713. No significant differences, other than a small gender difference, were detected between the genotype groups on admission to the

TABLE 4.15
Baseline characteristics of a cohort of Caucasian Subjects with sepsis by genotype of
Arginine Vasopressin (AVP) rs1410713 (AA vs. CC/AC). For age and APACHE II
score, data is given as 25^{th} percentile/median/75^{th} percentile. For all other
variables, data is given as % (N survived/N total).

| | AA | CC/AC | Combined | Test |
|---|---|---|---|---|
| | (N = 40) | (N = 524) | (N = 564) | Statistic |
| AGE | 48/60.5/73.3 | 46/59/71 | 47/59/72 | F = 1.26 d.f. = 1.562 P = 0.262 |
| GENDER | 48% (19/40) | 65% (338/524) | 63% (357/564) | X\hat{ }2 = 4.63 d.f. = 1 P = 0.0315 |
| APACHEII | 16/23.5/28.3 | 17/23/28 | 17/22/28 | F = 0.13 d.f. = 1.562 P = 0.715 |
| SURGICAL | 18% (7/40) | 23% (120/524) | 23% (127/564) | X\hat{ }2 = 0.62 d.f. = 1 P = 0.431 |
| SS. ADMIT | 57% (23/40) | 65% (343/524) | 65% (366/564) | X\hat{ }2 = 1.03 d.f. = 1 P = 0.309 |
| SS. ANY | 60% (24/40) | 69% (362/524) | 68% (386/564) | X\hat{ }2 = 1.42 d.f. = 1 P = 0.233 |

N, number of subjects.

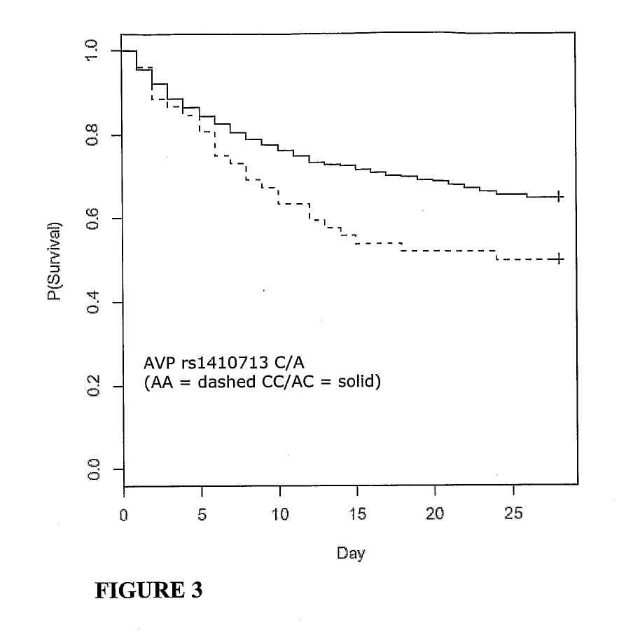

FIG. 3 and TABLE 4.16 summarize important SNP-phenotype associations for AVP rs1410713. Subjects with either the AVP rs1410713 CC or AC genotype had significantly increased survival (P=0.0325), significantly more days alive (P=0.0314) and significantly more days alive and free of acute renal dysfunction (P=0.0388). Subjects with either the AVP rs1410713 CC or AC genotype also had a strong trend for more days alive and free of coagulation dysfunction (P=0.0706), acute hepatic dysfunction (P=0.0783) and any hepatic dysfunction (P=0.0627). These findings indicate that Caucasian sepsis subjects who have either the CC or AC genotype at AVP rs1410713 have a lower risk of organ dysfunction (coagulation, renal and hepatic).

TABLE 4.16
Days alive and free of organ dysfunction (DAF) by genotype of Arginine Vasopressin (AVP)
rs1410713 (AA vs. CC/AC) in a cohort of Caucasian subjects with sepsis. For all variables
besides 28-day survival, data is given as 25^{th} percentile/median/75^{th} percentile.
For 28-day survival, data is given as % (N survived/N total).

| | AA | CC/AC | Combined | Test |
|---|---|---|---|---|
| | (N = 40) | (N = 524) | (N = 564) | Statistic |
| SURVIVAL | 52% (21/40) | 69% (361/524) | 68% (382/564) | X\hat{ }2 = 4.57 d.f. = 1 P = 0.0325 |
| DA | 6.75/28/28 | 15.75/28/28 | 15/28/28 | F = 4.65 d.f. = 1.562 P = 0.0314 |
| COAG.DAF | 4/22/28 | 11/28/28 | 10/28/28 | F = 3.28 d.f. = 1.562 P = 0.0706 |
| INR.DAF | (1.75/13.50/ | 8.75/27/28 | 8/27/28 | F = 7.7 d.f. = 1.562 P = 0.00571 |
| | 28 | | | |
| ACRF.DAF | 2/15.5/28 | 6/26/28 | 6/26/28 | F = 4.29 d.f. = 1.562 P = 0.0388 |
| ANYREN.DAF | 1.5/15.5/28 | 4/24/28 | 3/24.5/28 | F = 2.7 d.f. = 1.562 P = 0.101 |
| ACHEP.DAF | 6.75/23/28 | 9/28/28 | 9/28/28 | F = 3.11 d.f. = 1.562 P = 0.0783 |
| ANYHEP.DAF | 6/21/28 | 8/28/28 | 8/28/28 | F = 3.48 d.f. = 1.562 P = 0.0627 |

N, number of subjects.

### 2.2.1.3 Septic Shock—Caucasians

TABLE 4.17 summarizes the baseline characteristics (age, gender, APACHE II score and medical vs. surgical diagnosis) of 366 Caucasian septic shock subjects who were successfully genotyped at AVP rs1410713. No significant differences were detected between the genotype groups on admission to the ICU.

TABLE 4.17
Baseline characteristics of a cohort of Caucasian Subjects with septic
shock by genotype of Arginine Vasopressin (AVP) rs1410713
(AA vs. CC/AC). For age and APACHE II score, data is given as 25^{th}
percentile/median/75^{th} percentile. For all other variables,
data is given as % (N survived/N total).

| | AA | CC/AC | Combined | Test |
|---|---|---|---|---|
| | (N = 23) | (N = 343) | (N = 366) | Statistic |
| AGE | 50/67/75.5 | 48/62/72 | 48/62/73 | F = 1.16 d.f. = 1.364 |
| | | | | P = 0.283 |
| GENDER | 43% | 62% | 61% | X\hat{ }2 = 3.25 d.f. = 1 |
| | (10/23) | (214/343) | (224/366) | P = 0.0716 |
| APACHEII | 23.5/26/31 | 19.5/24/30 | 19/24/30 | F = 0.97 d.f. = 1.364 |
| | | | | P = 0.324 |
| SURGICAL | 13% | 25% | 25% | X\hat{ }2 = 1.76 d.f. = 1 |
| | (3/23) | (87/343) | (90/366) | P = 0.184 |

N, number of subjects.

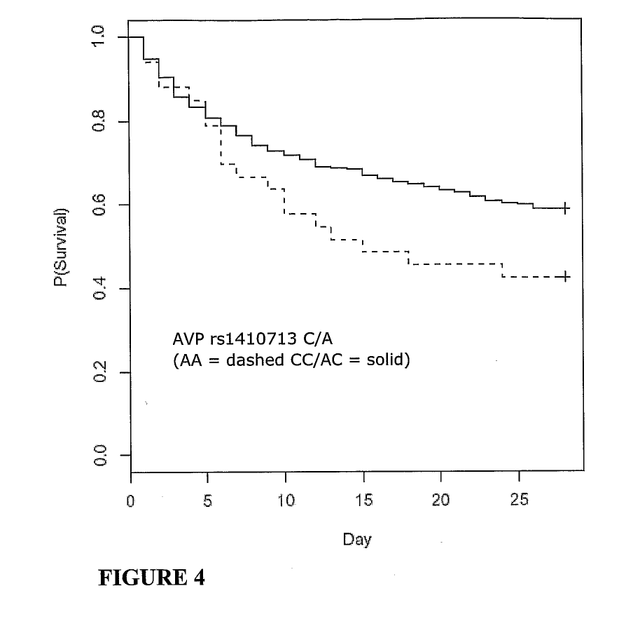

FIG. 4 and TABLE 4.18 summarize important SNP-phenotype associations for AVP rs1410713. Subjects with either the AVP rs1410713 CC or AC genotype had significantly increased survival (P=0.0269), significantly more days alive (P=0.0402) and significantly more days alive and free of 4 of 4 SIRS criteria (P=0.0445), acute renal dysfunction (P=0.0373) and INR>1.5 (P=0.00816). Subjects with either the AVP rs1410713 CC or AC genotype also had a strong trend for more days alive and free of vasopressors at doses of more than 2 ug/min (P=0.0982) and 5 ug/min (P=0.0982), inotropes (P=0.0962), coagulation dysfunction (P=0.0931), any renal dysfunction (P=0.0744) and any hepatic dysfunction (P=0.0619). These findings indicate that Caucasian septic shock subjects, who have either the CC or AC genotype at AVP rs1410713 have less need of vasopressor, and inotrope therapy, have less severe SIRS and have a lower risk of organ dysfunction (coagulation, renal and hepatic).

TABLE 4.18
Days alive and free of organ dysfunction (DAF) by genotype of Arginine Vasopressin (AVP)
rs1410713 (AA vs. CC/AC) in a cohort of Caucasian subjects with septic shock. For all variables
besides 28-day survival, data is given as 25^{th} percentile/median/75^{th} percentile. For 28-day
survival, data is given as % (N survived/N total).

| | AA | CC/AC | Combined | Test |
|---|---|---|---|---|
| | (N = 23) | (N = 343) | (N = 366) | Statistic |
| SURVIVAL | 39% (9/23) | 62% (214/343) | 61% (223/366) | X\hat{ }2 = 4.9 d.f. = 1 P = 0.0269 |
| DA | 6/15/28 | 9.5/28/28 | 9/28/28 | F = 4.24 d.f. = 1.364 P = 0.0402 |
| PRESS.DAF | 2/9/25 | 7/23/26 | 5.75/23/26 | F = 2.96 d.f. = 1.364 P = 0.086 |
| PRESS2.DAF | 2/9/25 | 7/23/26 | 5.75/23/26 | F = 2.75 d.f. = 1.364 P = 0.0982 |
| PRESS5.DAF | 2/10/25 | 7.5/24/27 | 6/24/27 | F = 2.75 d.f. = 1.364 P = 0.0982 |
| INO.DAF | 6/15/28 | 8/28/28 | 7/28/28 | F = 2.78 d.f. = 1.364 P = 0.0962 |
| MSIRS4.DAF | 3.5/11/26.5 | 7/24/27 | 7/23.5/27 | F = 4.06 d.f. = 1.364 P = 0.0445 |
| CSIRS4.DAF | 4.5/11/26.5 | 8/25/27 | 7/24/27 | F = 3.93 d.f. = 1.364 P = 0.0481 |
| COAG.DAF | 4/15/28 | 8/26/28 | 6/25/28 | F = 2.83 d.f. = 1.364 P = 0.0931 |
| INR.DAF | 0/7/26 | 6/23/28 | 5/24/28 | F = 7.08 d.f. = 1.364 P = 0.00816 |
| ACRF.DAF | 0/10/27 | 4/22/28 | 3/22/28 | F = 4.37 d.f. = 1.364 P = 0.0373 |
| ANYREN.DAF | 0/10/27 | 3/19/28 | 2.75/19.5/28 | F = 3.2 d.f. = 1.364 P = 0.0744 |
| ANYHEP.DAF | 5/12/26 | 6/28/28 | 5.75/26.5/28 | F = 3.51 d.f. = 1.364 P = 0.0619 |

N, number of subjects.

2.2.2 AVP rs857240

2.2.2.1 Sepsis—Caucasians

TABLE 4.19 gives the baseline characteristics (age, gender, APACHE II score, medical vs. surgical diagnosis, shock upon admission and septic shock anytime) of 573 Caucasian Subjects with sepsis who were successfully genotyped at AVP rs857240. No significant differences were detected between the genotype groups on admission to the ICU.

TABLE 4.19
Baseline characteristics of a cohort of Caucasian Subjects with sepsis by genotype of Arginine
Vasopressin (AVP) rs857240 (CC vs. CT/TT). For age and APACHE II score, data is given as
25^{th} percentile/median/75^{th} percentile. For all other variables, data is given as %
(N survived/N total).

| | CC | CT/TT | Combined | Test |
|---|---|---|---|---|
| | (N = 471) | (N = 102) | (N = 573) | Statistic |
| AGE | 46/59/71 | 43.3/55.5/71 | 47/59/72 | F = 0.57 d.f. = 1.571 P = 0.449 |
| GENDER | 63% (299/471) | 65% (66/102) | 64% (365/573) | X\hat{ }2 = 0.05 d.f. = 1 P = 0.816 |
| APACHEII | 17/23/28 | 15.3/21/27 | 17/22/28 | F = 2.84 d.f. = 1.571 P = 0.0926 |
| SURGICAL | 22% (103/471) | 25% (26/102) | 23% (129/573) | X\hat{ }2 = 0.63 d.f. = 1 P = 0.427 |
| SS. ADMIT | 64% (303/471) | 69% (70/102) | 65% (373/573) | X\hat{ }2 = 0.68 d.f. = 1 P = 0.409 |
| SS. ANY | 68% (321/471) | 72% (73/102) | 69% (394/573) | X\hat{ }2 = 0.46 d.f. = 1 P = 0.5 |

N, number of subjects.

TABLE 4.20 summarizes important SNP-phenotype associations for AVP rs857240. Subjects with either the AVP rs857240 TT or CT genotype had a trend for increased survival (P=0.0697), significantly more days alive (P=0.0398), significantly more days alive and free of inotropes (P=0.0457), coagulation dysfunction (P=0.0382). INR>10.5 (P=0.036), acute renal dysfunction (P=0.0238), any renal dysfunction (P=0.0087), renal support (P=0.0126), acute hepatic dysfunction (P=0.0292) and any hepatic dysfunction (P=0.0251). Subjects with either the AVP rs857240 TT or CT genotype also had a strong trend for more days alive and free of 4 of 4 SIRS criteria (P=0.0555). These findings indicate that Caucasian subjects who have sepsis who carry either the AVP rs857240 TT or CT genotype at AVP rs857240 have less need of inotrope therapy, have less severe SIRS, and have a lower risk of organ dysfunction (coagulation, renal and hepatic).

TABLE 4.20
Days alive and free of organ dysfunction (DAF) by genotype of Arginine Vasopressin (AVP)
rs857240 (CC vs. CT/TT) in a cohort of Caucasian subjects with sepsis. For all variables besides
28-day survival, data is given as 25^{th} percentile/median/75^{th} percentile. For 28-day survival,
data is given as % (N survived/N total).

| | CC | CT/TT | Combined | Test |
|---|---|---|---|---|
| | (N = 471) | (N = 102) | (N = 573) | Statistic |
| SURVIVAL | 66% (312/471) | 75% (77/102) | 68% (389/573) | X\hat{ }2 = 3.29 d.f. = 1 P = 0.0697 |
| DA | 11/28/28 | 28/28/28 | 15/28/28 | F = 4.24 d.f. = 1.571 |
| | | | | P = 0.0398 |
| INO.DAF | 11/28/28 | 25/28/28 | 12.3/28/28 | F = 4.01 d.f. = 1.571 |
| | | | | P = 0.0457 |
| MSIRS4.DAF | 8/25/28 | 20.3/26/28 | 11/25/28 | F = 3.68 d.f. = 1.571 |
| | | | | P = 0.0555 |
| CSIRS4.DAF | 9/26/28 | 21/26/28 | 11/26/28 | F = 3.15 d.f. = 1.571 |
| | | | | P = 0.0764 |
| COAG.DAF | 9.5/28/28 | 21/28/28 | 10/28/28 | F = 4.32 d.f. = 1.571 |
| | | | | P = 0.0382 |
| INR.DAF | 7/27/28 | 17.3/27/28 | 8/27/28 | F = 0.84 d.f. = 1.571 P = 0.036 |
| ACRF.DAF | 4.5/25/28 | 10.3/28/28 | 6/26/28 | F = 5.14 d.f. = 1.571 |
| | | | | P = 0.0238 |
| ANYREN.DAF | 3/22/28 | 10/28/28 | 3/24.5/28 | F = 6.94 d.f. = 1.571 |
| | | | | P = 0.00868 |
| RENSUP.DAF | 5/28/28 | 15/28/28 | 6/28/28 | F = 6.26 d.f. = 1.571 |
| | | | | P = 0.0126 |
| ACHEP.DAF | 7.5/28/28 | 19.3/28/28 | 9/28/28 | F = 4.78 d.f. = 1.571 |
| | | | | P = 0.0292 |
| ANYHEP.DAF | 6/28/28 | 18.3/28/28 | 8/28/28 | F = 5.04 d.f. = 1.571 |
| | | | | P = 0.0251 |

N, number of subjects.

2.2.2.2 Septic Shock—Caucasians

TABLE 4.21 summarizes the baseline characteristics (age, gender, APACHE II score and medical vs. surgical diagnosis) of 373 Caucasian septic shock subjects who were successfully genotyped at AVP rs857240. No significant differences were detected between the genotype groups on admission to the ICU.

TABLE 4.21
Baseline characteristics of a cohort of Caucasian Subjects with septic
shock by genotype of Arginine Vasopressin (AVP) rs857240
(CC vs. CT/TT). For age and APACHE II score, data is given as
25^{th} percentile/median/75^{th} percentile. For all other
variables, data is given as % (N survived/N total).

| | CC | CT/TT | Combined | Test |
|---|---|---|---|---|
| | (N = 303) | (N = 70) | (N = 373) | Statistic |
| AGE | 48/61/72 | 46.3/59/73.8 | 48/62/73 | F = 0 d.f. = 1.371 |
| | | | | P = 0.96 |
| GENDER | 62% | 61% (43/70) | 62% | X\hat{ }2 = 0 d.f. = 1 |
| | (187/303) | | (230/373) | P = 0.964 |
| APACHEII | 20.5/25/30 | 17.5/24/28 | 19/24/30 | F = 2.3 d.f. = 1.371 |
| | | | | P = 0.130 |
| SURGICAL | 23% | 31% (22/70) | 25% | X\hat{ }2 = 2.12 d.f. = 1 |
| | (70/303) | | (92/373) | P = 0.145 |

N, number of subjects.

TABLE 4.22 summarizes important SNP-phenotype associations for AVP rs857240. Subjects with either the AVP rs857240 TT or CT genotype had a trend for increased survival (P=0.0911, significantly more days alive (P=0.0467), significantly more days alive and free of inotropes (P=0.0416), acute renal dysfunction (P=0.0114), any renal dysfunction (P=0.0052), renal support (P=0.0266), acute hepatic dysfunction (P=0.0190) and any hepatic dysfunction (P=0.0115). Subjects with either the AVP rs857240 TT or CT genotype also had a strong trend for fewer days alive and free of vasopressors at doses of more than 5 ug/min (P=0.0895) and 15 ug/min (P=0.0747) and days alive and free of 4 of 4 SIRS criteria (P=0.0771). These findings indicate that Caucasian subjects with septic shock who had either the TT or CT genotype at AVP rs857240 have less need of vasopressor and inotrope therapy, have less SIRS, and have a lower risk of organ dysfunction (renal and hepatic).

TABLE 4.22
Days alive and free of organ dysfunction (DAF) by genotype of Arginine Vasopressin (AVP)
rs857240 (CC vs. CT/TT) in a cohort of Caucasian subjects with septic shock. For all variables
besides 28-day survival, data is given as 25^{th} percentile/median/75^{th} percentile.
For 28-day survival, data is given as % (N survived/N total).

| | CC | CT/TT | Combined | Test |
|---|---|---|---|---|
| | (N = 303) | (N = 70) | (N = 373) | Statistic |
| SURVIVAL | 59% | 70% (49/70) | 61% (228/373) | X\hat{ }2 = 2.86 d.f. = 1 P = 0.091 |
| | (179/303) | | | |
| DA | 8/28/28 | 22.5/28/28 | 9/28/28 | F = 3.98 d.f. = 1.371 P = 0.0467 |
| PRESS5.DAF | 5/23/27 | 16.5/25/27 | 6/24/27 | F = 2.9 d.f. = 1.371 P = 0.0895 |
| PRESS15.DAF | 6/26/28 | 19.8/27/28 | 8/26/28 | F = 3.2 d.f. = 1.371 P = 0.0747 |
| INO.DAF | 6/27/28 | 20.3/28/28 | 7/28/28 | F = 4.18 d.f. = 1.371 P = 0.0416 |
| MSIRS4.DAF | 6/23/27 | 15.3/25/27 | 7/23.5/27 | F = 3.14 d.f. = 1.371 P = 0.0771 |
| ACRF.DAF | 3/20/28 | 10/27.5/28 | 3/22/28 | F = 6.47 d.f. = 1.371 P = 0.0114 |
| ANYREN.DAF | 1.50/18/28 | 9.25/27/28 | 2.75/19.50/28 | F = 7.91 d.f. = 1.371 P = 0.00517 |
| RENSUP.DAF | 3/24/28 | 12.5/28/28 | 4/25/28 | F = 4.95 d.f. = 1.371 P = 0.0266 |
| ACHEP.DAF | 5.5/27/28 | 17.3/28/28 | 6/28/28 | F = 5.55 d.f. = 1.371 P = 0.0190 |
| ANYHEP.DAF | 5/24/28 | 16.25/28/28 | 5.75/26.5/28 | F = 6.45 d.f. = 1.371 P = 0.0115 |

N, number of subjects.

2.2.3 AVP rs857242

2.2.3.1 Systematic Inflammatory Response Syndrome—Caucasians

TABLE 4.23 summarizes the baseline characteristics (age, gender, APACHE II score, medical vs. surgical diagnosis, sepsis upon admission, sepsis anytime, septic shock upon admission and septic shock anytime) of 722 Caucasian systematic inflammatory response syndrome subjects who were successfully genotyped at AVP rs857242. Significant differences were detected between the genotype groups on admission to the ICU (APACHE II).

TABLE 4.23
Baseline characteristics of a cohort of Caucasian Subjects with systematic inflammatory response
syndrome by genotype of Arginine Vasopressin (AVP) rs857242 (AC/AA vs. CC). For age and
APACHE II score, data is given as 25^{th} percentile/median/75^{th} percentile. For all other
variables, data is given as % (N survived/N total).

| | AC/AA | CC | Combined | Test |
|---|---|---|---|---|
| | (N = 154) | (N = 568) | (N = 722) | Statistic |
| AGE | 43.3/56/69.8 | 45/59.5/71 | 46/59/71 | F = 1.93 d.f. = 1.720 P = 0.165 |
| GENDER | 64% (98/154) | 61% (349/568) | 62% (447/722) | X\hat{ }2 = 0.25 d.f. = 1 P = 0.619 |
| APACHEII | 15/20/26 | 16/22/28 | 16/21.5/27 | F = 4.63 d.f. = 1.720 |
| | | | | P = 0.0317 |
| SURGICAL | 25% (39/154) | 22% (124/568) | 23% (163/722) | X\hat{ }2 = 0.85 d.f. = 1 P = 0.358 |
| SEP.ADMIT | 73% (112/154) | 80% (454/568) | 78% (566/722) | X\hat{ }2 = 3.71 d.f. = 1 P = 0.0541 |
| SEP.ANY | 74% (114/154) | 82% (465/568) | 80% (579/722) | X\hat{ }2 = 4.69 d.f. = 1 P = 0.0304 |
| SS.ADMIT | 49% (76/154) | 51% (292/568) | 51% (368/722) | X\hat{ }2 = 0.21 d.f. = 1 P = 0.65 |
| SS.ANY | 53% (82/154) | 55% (312/568) | 55% (394/722) | X\hat{ }2 = 0.14 d.f. = 1 P = 0.71 |

N, number of subjects.

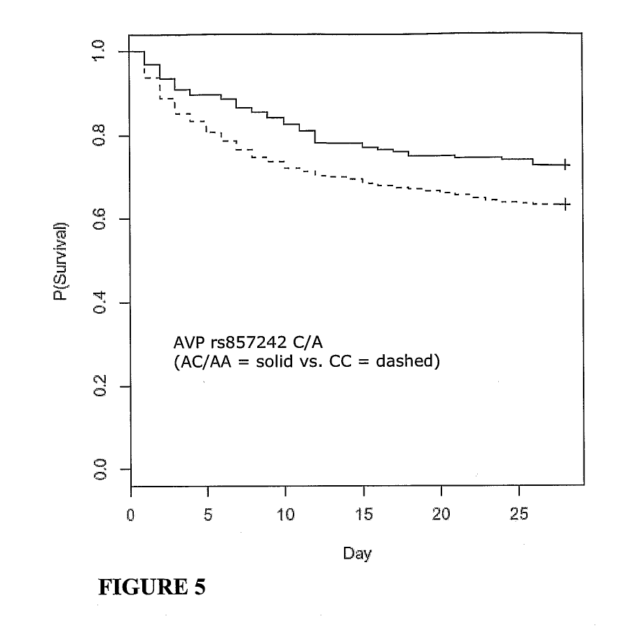

FIG. 5 and TABLE 4.24 summarize important SNP-phenotype associations for AVP rs857242. Subjects with either the AVP rs857242 AC or AA genotype had significantly increased survival (P=0.0108), significantly more days alive (P=0.0032) and significantly more days alive and free of vasopressors at doses of more than 5 ug/min (P=0.0361) and 15 ug/min (P=0.0026), days alive and free of inotropes (P=0.0394), 3 of 4 SIRS criteria (P=0.0170), 4 of 4 SIRS criteria (P=0.0043), neurological dysfunction (P=0.033), coagulation dysfunction (P<0.001), acute renal dysfunction (P=0.0341), any renal dysfunction (P=0.0127), renal support (P=0.0017), acute hepatic dysfunction (P=0.0013) and any hepatic dysfunction (P=0.0021). The AVP rs857242 AC or AA individuals also showed a strong trend for days alive and free of vasopressors (P=0.0752), days alive and free of vasopressors at a dose of more than 2 ug/min (P=0.0524), 2 of 4 SIRS criteria (P=0.059), INR>1.5 (P=0.0679). These findings indicate that Caucasian subjects with SIRS who had either the AC or AA genotype at AVP rs857242 have less need of vasopressor and inotrope therapy, have less severe SIRS and have a lower risk of organ dysfunction (neurological, coagulation, renal and hepatic).

TABLE 4.24
Days alive and free of organ dysfunction (DAF) by genotype of Arginine Vasopressin (AVP)
rs857242 (AC/AA vs. CC) in a cohort of Caucasian subjects with systematic inflammatory
response syndrome. For all variables besides 28-day survival, data is given as 25^{th} percentile/
median/75^{th} percentile. For 28-day survival, data is given as % (N survived/N total).

| | AC/AA | CC | Combined | Test |
|---|---|---|---|---|
| | (N = 154) | (N = 568) | (N = 722) | Statistic |
| SURVIVAL | 77% (119/154) | 67% (378/568) | 69% (497/722) | X\hat{ }2 = 6.49 d.f. = 1 P = 0.0108 |
| DA | 28/28/28 | 9.75/28/28 | 12/28/2 | F = 8.74 d.f. = 1.720 P = 0.00321 |
| PRESS.DAF | 18.3/26/28 | 7/26/28 | 9/26/28 | F = 3.18 d.f. = 1.720 P = 0.0752 |
| PRESS2.DAF | 18.3/26/28 | 7/26/28 | 10/26/28 | F = 3.78 d.f. = 1.720 P = 0.0524 |
| PRESS5.DAF | 20.25/27/28 | 7.75/26/28 | 10/26/28 | F = 4.41 d.f. = 1.720 P = 0.0361 |
| PRESS15.DAF | 25/28/28 | 9/28/28 | 12/28/28 | F = 9.16 d.f. = 1.720 P = 0.00256 |
| INO.DAF | 25/28/28 | 8/28/28 | 11.3/28/28 | F = 4.26 d.f. = 1.720 P = 0.0394 |
| MSIRS2.DAF | 1/6/22 | 0/4/20.3 | 0/5/21 | F = 3.58 d.f. = 1.720 P = 0.059 |
| MSIRS3.DAF | 7.25/22/26 | 2/19/26 | 3/19/26 | F = 5.72 d.f. = 1.720 P = 0.0170 |
| MSIRS4.DAF | 19.50/27/28 | 7/26/28 | 9.25/26/28 | F = 8.19 d.f. = 1.720 P = 0.00434 |
| CSIRS2.DAF | 1/5.5/22 | 0/4/20 | 0/5/20 | F = 3.23 d.f. = 1.720 P = 0.0726 |
| CSIRS3.DAF | 8/22/26 | 3/19/26 | 4/20/26 | F = 5.84 d.f. = 1.720 P = 0.0159 |
| CSIRS4.DAF | 21/27/28 | 8/26/28 | 10/26/28 | F = 8.22 d.f. = 1.720 P = 0.00427 |
| CNS.DAF | 18.25/27/28 | 5.75/27/28 | 7.25/27/28 | F = 4.56 d.f. = 1.720 P = 0.033 |
| COAG.DAF | 21.25/28/28 | 7/28/28 | 8.25/28/28 | F = 11.6 d.f. = 1.720 P < 0.001 |
| INR.DAF | 15/28/28 | 5/27/28 | 7/27/28 | F = 3.34 d.f. = 1.720 P = 0.0679 |
| ACRF.DAF | 10/28/28 | 4/26/28 | 5/27/28 | F = 4.51 d.f. = 1.720 P = 0.0341 |
| ANYREN.DAF | 9/28/28 | 2/23/28 | 3/25/28 | F = 6.25 d.f. = 1.720 P = 0.0127 |
| RENSUP.DAF | 15/28/28 | 4/28/28 | 5/28/28 | F = 9.92 d.f. = 1.720 P = 0.00171 |
| ACHEP.DAF | 21/28/28 | 6.75/28/28 | 8/28/28 | F = 10.4 d.f. = 1.720 P = 0.00132 |
| ANYHEP.DAF | 18.8/28/28 | 6/28/28 | 7/28/28 | F = 9.52 d.f. = 1.720 P = 0.00211 |

N, number of subjects.

2.2.3.2 Sepsis—Caucasians

TABLE 4.25 gives the baseline characteristics (age, gender, APACHE II score, medical vs. surgical diagnosis, shock upon admission and septic shock anytime) of 567 Caucasian sepsis subjects who were successfully genotyped at AVP rs857242. No significant differences were detected between the genotype groups on admission to the ICU.

TABLE 4.25
Baseline characteristics of a cohort of Caucasian Subjects with sepsis by genotype of Arginine
Vasopressin (AVP) rs857242 (AC/AA vs. CC). For age and APACHE II score, data is given as
25^{th} percentile/median/75^{th} percentile. For all other variables, data is given as %
(N survived/N total).

| | AC/AA | CC | Combined | Test |
|---|---|---|---|---|
| | (N = 112) | (N = 455) | (N = 567) | Statistic |
| AGE | 44/56/69.3 | 46/60/71 | 47/59/72 | F = 1.05 d.f. = 1.565 P = 0.306 |
| GENDER | 63% (71/112) | 64% (289/455) | 63% (360/567) | X\hat{ }2 = 0 d.f. = 1 P = 0.98 |
| APACHEII | 16/21/27 | 17.5/23/28 | 17/22/28 | F = 2.8 d.f. = 1.565 P = 0.0945 |
| SURGICAL | 27% (30/112) | 21% (96/455) | 22% (126/567) | X\hat{ }2 = 1.68 d.f. = 1 P = 0.195 |
| SS.ADMIT | 68% (76/112) | 64% (292/455) | 65% (368/567) | X\hat{ }2 = 0.53 d.f. = 1 P = 0.465 |
| SS.ANY | 72% (81/112) | 68% (308/455) | 69% (389/567) | X\hat{ }2 = 0.89 d.f. = 1 P = 0.344 |

N, number of subjects.

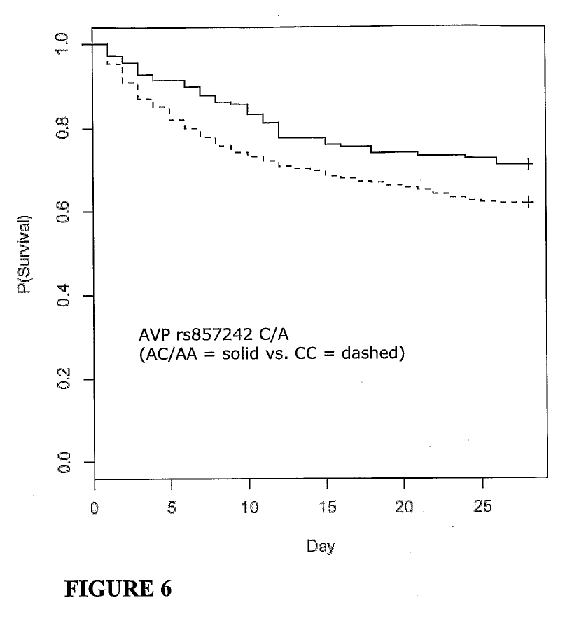

FIG. 6 and TABLE 4.26 summarize important SNP-phenotype associations for AVP rs857242. Subjects with either the AVP rs857242 AC or AA genotype had significantly increased survival (P=0.0220), significantly more days alive (P=0.0059) and significantly days alive and free of vasopressors at a dose of more than 15 ug/min (P=0.0078), 3 of 4 SIRS criteria (P=0.0219), 4 of 4 SIRS criteria (P=0.0058), coagulation dysfunction (P=0.0012), acute renal dysfunction (P=0.0116), any renal dysfunction (P=0.0089), renal support (P=0.0104), acute hepatic dysfunction (P=0.0013) and any hepatic dysfunction (P=0.0014). Subjects with either the AVP rs857242 AC or AA genotype also had a strong trend for more days alive and free of inotropes (P=0.0646) INR>1.5 (P=0.0636) and neurological dysfunction (P=0.0803). These findings indicate that Caucasian subjects with sepsis who had either the AVP rs857242 AC or AA genotype at AVP rs857242 have less need of vasopressor and inotrope therapy, have less severe SIRS and are have a lower risk of organ dysfunction (neurological, coagulation, renal and hepatic).

TABLE 4.26
Days alive and free of organ dysfunction (DAF) by genotype of Arginine Vasopressin (AVP)
rs857242 (AC/AA vs. CC) in a cohort of Caucasian subjects with sepsis. For all variables besides
28-day survival, data is given as 25^{th} percentile/median/75^{th} percentile.
For 28-day survival, data is given as % (N survived/N total).

| | AC/AA | CC | Combined | Test |
|---|---|---|---|---|
| | (N = 112) | (N = 455) | (N = 567) | Statistic |
| SURVIVAL | 77% (86/112) | 65% (298/455) | 68% (384/567) | X\hat{ }2 = 5.24 d.f. = 1 P = 0.0220 |
| DA | 28/28/28 | 10/28/28 | 15/28/28 | F = 762 d.f. = 1.565 P = 0.00595 |
| PRESS15.DAF | 24.8/28/28 | 9.5/27/28 | 13/27.5/28 | F = 7.13 d.f. = 1.565 P = 0.00779 |
| INO.DAF | 24.8/28/28 | 9/28/28 | 12.3/28/28 | F = 3.43 d.f. = 1.565 P = 0.0646 |
| MSIRS3.DAF | 8/19/26 | 2/16/25 | 3/17/25 | F = 5.29 d.f. = 1.565 P = 0.0219 |
| MSIRS4.DAF | 19/27/28 | 8/25/28 | 11/25/28 | F = 7.66 d.f. = 1.565 P = 0.00582 |
| CSIRS3.DAF | 8/21/26 | 3/17/25 | 4/19/25 | F = 5.32 d.f. = 1.565 P = 0.0214 |
| CSIRS4.DAF | 21/27/28 | 8/25/28 | 11/26/28 | F = 6.87 d.f. = 1.565 P = 0.00902 |
| CNS.DAF | 18/26/28 | 7/26/28 | 8/26/28 | F = 3.07 d.f. = 1.565 P = 0.0803 |
| COAG.DAF | 22/28/28 | 8.5/28/28 | 10/28/28 | F = 10.6 d.f. = 1.565 P = 0.00123 |
| INR.DAF | 15.8/27.5/28 | 6/26/28 | 8/27/28 | F = 3.46 d.f. = 1.565 P = 0.0636 |
| ACRF.DAF | 11/28/28 | 4/25/28 | 6/26/28 | F = 6.42 d.f. = 1.565 P = 0.0116 |
| ANYREN.DAF | 10/28/28 | 3/22/28 | 3/24.5/28 | F = 6.9 d.f. = 1.565 P = 0.00887 |
| RENSUP.DAF | 15/28/28 | 5/28/28 | 6/28/28 | F = 6.61 d.f. = 1.565 P = 0.0104 |
| ACHEP.DAF | 21.8/28/28 | 7/28/28 | 9/28/28 | F = 10.4 d.f. = 1.565 P = 0.00131 |
| ANYHEP.DAF | 21/28/28 | 6/28/28 | 8/28/28 | F = 10.2 d.f. = 1.565 P = 0.00145 |

N, number of subjects.

2.2.3.3 Septic Shock—Caucasians

TABLE 4.27 summarizes the baseline characteristics (age, gender, APACHE II score and medical vs. surgical diagnosis) of 368 Caucasian septic shock subjects who were successfully genotyped at AVP rs857242. No significant differences were detected between the genotype groups on admission to the ICU.

TABLE 4.27
Baseline characteristics of a cohort of Caucasian Subjects with septic shock by genotype of
Arginine Vasopressin (AVP) rs857242 (AC/AA vs. CC). For age and APACHE II score, data is
given as 25^{th} percentile/median/75^{th} percentile. For all other variables, data is given as % (N
survived/N total).

| | AC/AA | CC | Combined | Test |
|---|---|---|---|---|
| | (N = 76) | (N = 292) | (N = 368) | Statistic |
| AGE | 44.8/57/71 | 48/63/72 | 48/62/73 | F = 1.28 d.f. = 1.366 P = 0.258 |
| GENDER | 59% (45/76) | 62% (181/292) | 61% (226/368) | X\hat{ }2 = 0.2 d.f. = 1 P = 0.658 |
| APACHEII | 17/24/28.3 | 20.8/25/3 | 19/24/30 | F = 2.52 d.f. = 1.366 P = 0.113 |
| SURGICAL | 32% (24/76) | 22% (65/292) | 24% (89/368) | X\hat{ }2 = 2.86 d.f. = 1 P = 0.091 |

N, number of subects.

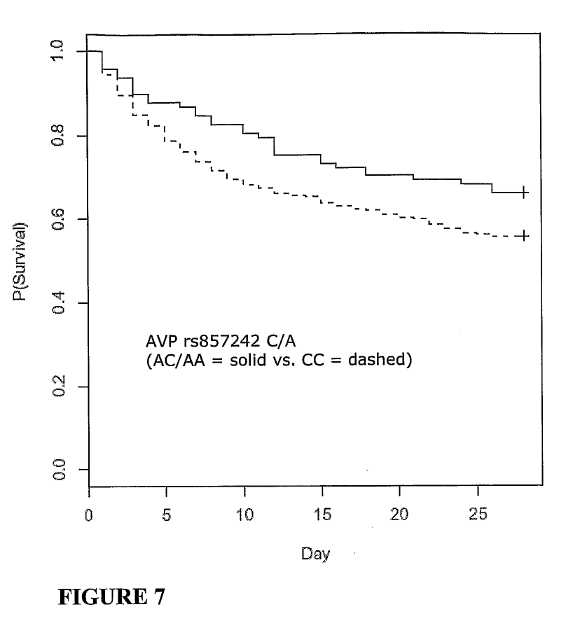

FIG. 7 and TABLE 4.28 summarize important SNP-phenotype associations for AVP rs857242. Subjects with either the AVP rs857242 AC or AA genotype had significantly increased survival (P=0.0466), significantly more days alive (P=0.0129) and significantly more days alive and free of vasopressors at a dose of more than 15 ug/min (P=0.0032), 4 of 4 SIRS criteria (P=0.0146), neurological dysfunction (P=0.0365) coagulation dysfunction (P=0.0027), acute renal dysfunction (P=0.0103), any renal dysfunction (P=0.0063), renal support (P=0.0165), acute hepatic dysfunction (P=0.0013) and any hepatic dysfunction (P<0.001). Subjects with either the AVP rs857242 AC or AA genotype also had a strong trend for days alive and free of vasopressors at doses of more than 2 ug/min (P=0.0839) and 5 ug/min (P=0.054), INR>1.5 (P=0.0549) and inotropes (P=0.0592). These findings indicate that Caucasian subjects with septic shock who had either the AC or AA genotype at AVP rs857242 had less need of vasopressor, and inotrope therapy, had more sever SIRS and had a lower risk of organ dysfunction (neurological, coagulation, renal and hepatic).

TABLE 4.28
Days alive and free of organ dysfunction (DAF) by genotype of Arginine Vasopressin (AVP)
rs857242 (AC/AA vs. CC) in a cohort of Caucasian subjects with septic shock. For all variables
besides 28-day survival, data is given as 25^{th} percentile/median/75^{th} percentile. For 28-day
survival, data is given as % (N survived/N total).

| | AC/AA | CC | Combined | Test |
|---|---|---|---|---|
| | (N = 76) | (N = 292) | (N = 368) | Statistic |
| SURVIVAL | 71% (54/76) | 59% (171/292) | 61% (225/368) | X\hat{ }2 = 3.96 d.f. = 1 P = 0.0466 |
| DA | 23.3/28/28 | 7/28/28 | 9/28/28 | F = 6.25 d.f. = 1.366 P = 0.0129 |
| PRESS.DAF | 13.75/24/26 | 4/22/26 | 5.75/23/26 | F = 2.91 d.f. = 1.366 P = 0.089 |
| PRESS2.DAF | 13.75/24/26 | 4/22/26 | 5.75/23/26 | F = 3 d.f. = 1.366 P = 0.0839 |
| PRESS5.DAF | 15.8/25/27 | 5/23/27 | 6/24/27 | F = 3.74 d.f. = 1.366 P = 0.054 |
| PRESS15.DAF | 21/27/28 | 6/26/28 | 8/26/28 | F = 8.81 d.f. = 1.366 P = 0.0032 |
| INO.DAF | 19.8/28/28 | 6/28/28 | 7/28/28 | F = 3.58 d.f. = 1.366 P = 0.0592 |
| MSIRS2.DAF | 0.75/2.50/ | 0/2/15 | 0/2/16 | F = 3.08 d.f. = 1.366 P = 0.0803 |
| | 17.75 | | | |
| MSIRS3.DAF | 6.75/15.50/25 | 1/12/23 | 2/13/23 | F = 4.42 d.f. = 1.366 P = 0.0362 |
| MSIRS4.DAF | 14.25/25/28 | 5.75/23/27 | 7/23.50/27 | F = 6.02 d.f. = 1.366 P = 0.0146 |
| CSIRS3.DAF | 6/16/25 | 2/13.5/23.3 | 3/14/24 | F = 3.79 d.f. = 1.366 P = 0.0525 |
| CSIRS4.DAF | 15.8/25/28 | 6/24/27 | 7/24/27.3 | F = 5.22 d.f. = 1.366 P = 0.0229 |
| CNS.DAF | 14.8/25.5/28 | 5/24/28 | 6/25/28 | F = 4.41 d.f. = 1.366 P = 0.0365 |
| COAG.DAF | 15/28/28 | 6/24/28 | 6/25/28 | F = 9.15 d.f. = 1.366 P = 0.00266 |
| INR.DAF | 14.8/25.5/28 | 3/22/28 | 5/24/28 | F = 3.71 d.f. = 1.366 P = 0.0549 |
| ACRF.DAF | 10/27.5/28 | 3/20/28 | 3/22/28 | F = 6.65 d.f. = 1.366 P = 0.0103 |
| ANYREN.DAF | 9.75/27/28 | 2/18/28 | 2.75/19.5/28 | F = 7.55 d.f. = 1.366 P = 0.00628 |
| RENSUP.DAF | 13.5/28/28 | 3/24/28 | 4/25/28 | F = 5.81 d.f. = 1.366 P = 0.0165 |
| ACHEP.DAF | 17.5/28/28 | 5/25.5/28 | 6/28/28 | F = 10.4 d.f. = 1.366 P = 0.00134 |
| ANYHEP.DAF | 16/28/28 | 5/23.50/28 | 5.75/26.5/28 | F = 11.7 d.f. = 1.366 P < 0.001 |

N, number of subjects.

### Arginine Vasopressin Receptor 1a (AVPR1A)

2.3.1 AVPR1A rs1495027

2.3.1.1 Septic Shock—Caucasians

TABLE 4.29 gives the baseline characteristics (age, gender, APACHE II score, and medical vs. surgical diagnosis) of the 361 Caucasian septic shock subjects who were successfully genotyped at AVPR1A rs1495027 (CC vs. CT/TT). No significant differences were detected between the two genotype groups on admission to the ICU.

TABLE 4.29
Baseline characteristics of a cohort of Caucasian Subjects with septic shock by genotype of
arginine vasopressin receptor 1a (AVPR1A) rs1495027 (CC vs. CT/TT). For age and APACHE II
score, data is given as 25^{th} percentile/median/75^{th} percentile. For all other variables, data is
given as % (N survived/N total).

| | CC | CT/TT | Combined | Test |
|---|---|---|---|---|
| | (N = 129) | (N = 232) | (N = 361) | Statistic |
| AGE | 47/61/72 | 48.8/61.5/ | 48/62/73 | F = 0.42 d.f. = 1.359 P = 0.516 |
| | | 72.3 | | |
| GENDER | 59% (76/129) | 64% | 62% (224/361) | X\hat{ }2 = 0.84 d.f. = 1 P = 0.36 |
| | | (148/232) | | |
| APACHEII | 19/25/31 | 20/25/29 | 19/24/30 | F = 0.01 d.f. = 1.359 P = 0.918 |
| SURGICAL | 22% (28/129) | 25% (59/232) | 24% (87/361) | X\hat{ }2 = 0.63 d.f. = 1 P = 0.428 |

N, number of subjects.

TABLE 4.30 summarizes important SNP-phenotype associations for AVPR1A rs1495027. Subjects with either the AVPR1A rs1495027 CT or TT genotype had significantly more days alive and free of renal support (P=0.0325). Subjects with either the AVPR1A rs1495027 CT or TT genotype also had a strong trend for more days alive and free of vasopressors at a dose of 5 ug/min (P=0.0832) and 2 of 4 SIRS criteria (P=0.0958). These findings indicate that Caucasian subjects with septic shock with the CT or TT genotype at AVPR1A rs1495027 have less need of vasopressors and have decreased risk of SIRS and organ dysfunction (renal).

TABLE 4.30
Days alive and free of organ dysfunction (DAF)
by genotype of arginine vasopressin receptor 1a (AVPR1A)
rs1495027 (CC vs. CT/TT) in a cohort of Caucasian subjects
with septic shock. Data is given as
25^{th} percentile/median/75^{th} percentile.

| | CC | CT/TT | Combined | Test |
|---|---|---|---|---|
| | (N = 129) | (N = 232) | (N = 361) | Statistic |
| PRESS5.DAF | 5/23/26 | 8/24/27 | 6/24/27 | F = 3.02 |
| | | | | d.f. = 1.359 |
| | | | | P = 0.0832 |
| MSIRS2.DAF | 0/1/13 | 0/2/16 | 0/2/16 | F = 2.99 |
| | | | | d.f. = 1.359 |
| | | | | P = 0.0845 |
| RENSUP.DAF | 3/18/28 | 6/28/28 | 4/25/28 | F = 4.61 |
| | | | | d.f. = 1.359 |
| | | | | P = 0.0325 |

N, number of subjects.

2.3.2 AVPR1A rs3803107

2.3.2.1 Systematic Inflammatory Response Syndrome—Caucasians

TABLE 4.31 gives the baseline characteristics (age, gender, APACHE II score, medical vs. surgical diagnosis, sepsis upon admission, sepsis anytime, septic shock upon admission and septic shock anytime) of the 729 Caucasian SIRS subjects who were successfully genotyped (CT/TT vs. CC) at AVPR1A rs3803107. No significant differences were detected between the two genotype groups on admission to the ICU.

TABLE 4.31
Baseline characteristics of a cohort of Caucasian Subjects with systematic inflammatory response
syndrome by genotype of arginine vasopressin receptor 1a (AVPR1A) rs3803107 (CC/CT vs.
TT). For age and APACHE II score, data is given as 25^{th} percentile/median/75^{th} percentile. For
all other variables, data is given as % (N survived/N total).

| | CC/CT | TT | Combined | Test |
|---|---|---|---|---|
| | (N = 706) | (N = 23) | (N = 729) | Statistic |
| AGE | 44/58/71 | 47.5/61/69 | 46/59/71 | F = 0.01 d.f. = 1.727 P = 0.934 |
| GENDER | 62% (435/706) | 65% (15/23) | 62% (450/729) | X\hat{ }2 = 0.12 d.f. = 1 P = 0.726 |
| APACHEII | 16/22/27 | 19/25/27.5 | 16/21.5/27 | F = 2 d.f. = 1.727 P = 0.157 |
| SURGICAL | 23% (159/706) | 22% (5/23) | 22% (164/729) | X\hat{ }2 = 0.01 d.f. = 1 P = 0.93 |
| SEP.ADMIT | 78% (549/706) | 87% (20/23) | 78% (569/729) | X\hat{ }2 = 1.1 d.f. = 1 P = 0.294 |
| SEP.ANY | 80% (562/706) | 87% (20/23) | 80% (582/729) | X\hat{ }2 = 0.75 d.f. = 1 P = 0.387 |
| SS.ADMIT | 50% (354/706) | 70% (16/23) | 51% (370/729) | X\hat{ }2 = 3.36 d.f. = 1 P = 0.0667 |
| SS.ANY | 54% (380/706) | 70% (16/23) | 54% (396/729) | X\hat{ }2 = 2.22 d.f. = 1 P = 0.136 |

N, number of subjects.

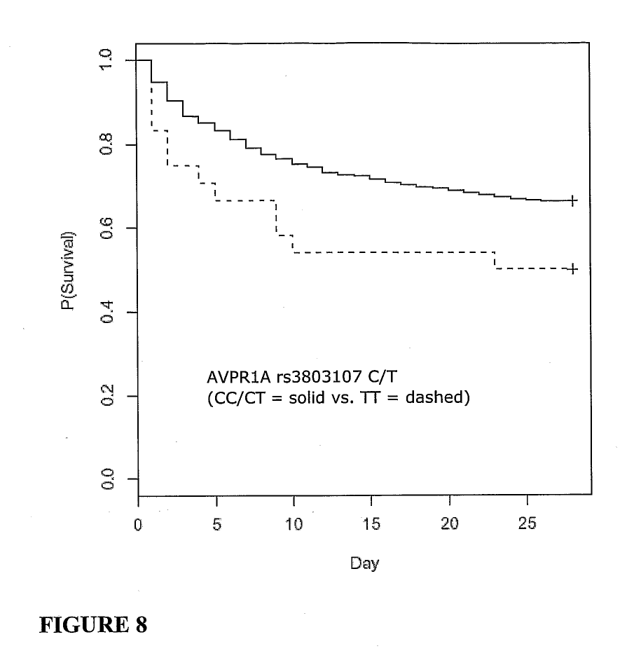

FIG. 8 and TABLE 4.32 summarize important SNP-phenotype association results for AVPR1A rs3803107. Subjects with either the AVPR1A rs3803107 CC or CT genotype had a strong trend for increased 28-day survival (P=0.0709) and significantly more days alive (P=0.0468) and significantly more days alive and free of vasopressors (P=0.0270), more days alive and free of vasopressors at doses of 2 ug/min (P=0.0286) and 5 ug/min (P=0.0163), cardiovascular dysfunction (P=0.0304) and respiratory dysfunction (P=0.0476). Subjects with either the AVPR1A rs3803107 CC or CT genotype also had a strong trend for more days alive and free of inotropes (P=0.0966). 4 of 4 SIRS criteria (P=0.0621), mechanical ventilation (P=0.0763) and acute hepatic dysfunction (P=0.0871). These findings indicate that Caucasian subjects with SIRS who had either the CC or CT genotype at AVPR1A rs3803107 have less need of vasopressors therapy and have decreased risk of SIRS and organ dysfunction (cardiovascular, respiratory, and hepatic).

TABLE 4.32
Days alive and free of organ dysfunction (DAF) by
genotype of arginine vasopressin receptor a (AVPR1A) rs3803107
(CC/CT vs. TT) in a cohort of Caucasian subjects with systematic inflammatory response
syndrome. For all variables besides 28-day survival, data is given
as 25^{th} percentile/median/75^{th} percentile.
For 28-day survival, data is given as % (N survived/N total).

| | CC/CT | TT | Combined | Test |
|---|---|---|---|---|
| | (N = 706) | (N = 23) | (N = 729) | Statistic |
| SURVIVAL | 70% | 52% (12/706) | 69% | X\hat{ }2 = 3.26 d.f. = 1 P = 0.0709 |
| | (493/706) | | (505/706) | |
| DA | 15/28/28 | 4.5/28/28 | 12/28/28 | F = 3.97 d.f. = 1.727 P = 0.0468 |
| ALI.DAF | 5/24.5/28 | 2/9/27.5 | 4/24/28 | F = 3.41 d.f. = 1.727 P = 0.651 |
| PRESS.DAF | 10.3/26/ | 3/22/26 | 9/26/28 | F = 4.91 d.f. = 1.727 P = 0.0270 |
| | 28 | | | |
| PRESS2.DAF | 11/26/28 | 3/22/26 | 10/26/28 | F = 4.81 d.f. = 1.727 P = 0.0286 |
| PRESS5.DAF | 11.3/26/ | 3/25/26 | 10/16/28 | F = 5.8 d.f. = 1.727 P = 0.0163 |
| | 28 | | | |
| INO.DAF | 14/28/28 | 4/23/28 | 11.3/28/28 | F = 2.77 d.f. = 1.727 P = 0.0966 |
| MSIRS4.DAF | 11/26/28 | 3.50/16/ | 9.25/26/28 | F = 3.49 d.f. = 1.727 P = 0.0621 |
| | | 27.50 | | |
| CSIRS4.DAF | 11/26/28 | 3.5/16/28 | 10/16/28 | F = 3.46 d.f. = 1.727 P = 0.0632 |
| CVS.DAF | 6/23/27 | 2.5/8/24.5 | 5/23/27 | F = 4.7 d.f. = 1.727 P = 0.0304 |
| RESP.DAF | 1/21/27 | 1/6/20.5 | 1/20/26 | F = 3.94 d.f. = 1.727 P = 0.0476 |
| PF300.DAF | 0/1/11 | 0/0/2 | 0/1/10 | F = 3.11 d.f. = 1.727 P = 0.0783 |
| VENT.DAF | 0/19/26 | 0/6/20.5 | 0/19/26 | F = 3.15 d.f. = 1.727 P = 0.0763 |
| ACHEP.DAF | 8.25/28/2 | 4.50/10/28 | 8/28/28 | F = 2.94 d.f. = 1.727 P = 0.0871 |

N, number of subjects.

2.3.2.2 Systematic Inflammatory Response Syndrome—Asians

TABLE 4.33 summarizes the baseline characteristics (age, gender, APACHE II score, medical vs. surgical diagnosis, sepsis upon admission, sepsis anytime, septic shock upon admission and septic shock anytime) of the 108 Asian SIRS subjects who were successfully genotyped (C vs. T) at AVPR1A rs3803107. No significant differences were detected between the two allelic groups on admission to the ICU.

TABLE 4.33
Baseline characteristics of a cohort of Asian Subjects with systematic inflammatory response
syndrome by allele of arginine vasopressin receptor 1a (AVPR1A) rs3803107 (C vs. T). For age
and APACHE II score, data is given as 25^{th} percentile/median/75^{th} percentile. For all other
variables, data is given as % (N survived/N total).

| | C | T | Combined | Test |
|---|---|---|---|---|
| | (N = 186) | (N = 30) | (N = 216) | Statistic |
| AGE | 51/68/76 | 58/71.5/76.8 | 54.5/69/76 | F = 0.57 d.f. = 1.214 P = 0.451 |
| GENDER | 61% (114/186) | 47% (14/30) | 59% (128/216) | X\hat{ }2 = 2.29 d.f. = 1 P = 0.130 |
| APACHEII | 17/22.5/29 | 19/25.5/33.5 | 17/23/30 | F = 3.12 d.f. = 1.214 P = 0.0787 |
| SURGICAL | 24% (44/186) | 13% (4/30) | 22% (48/216) | X\hat{ }2 = 1.59 d.f. = 1 P = 0.207 |
| SEP.ADMIT | 77% (143/186) | 77% (23/30) | 77% (166/216) | X\hat{ }2 = d.f. = 1 P = 0.98 |
| SEP.ANY | 80% (148/186) | 80% (24/30) | 80% (172/216) | X\hat{ }2 = 0 d.f. = 1 P = 0.957 |
| SS.ADMIT | 55% (102/186) | 53% (16/30) | 55% (118/216) | X\hat{ }2 = 0.02 d.f. = 1 P = 0.878 |
| SS.ANY | 63% (118/186) | 60% (18/30) | 63% (136/216) | X\hat{ }2 = 0.13 d.f. = 1 P = 0.717 |

N, number of subjects.

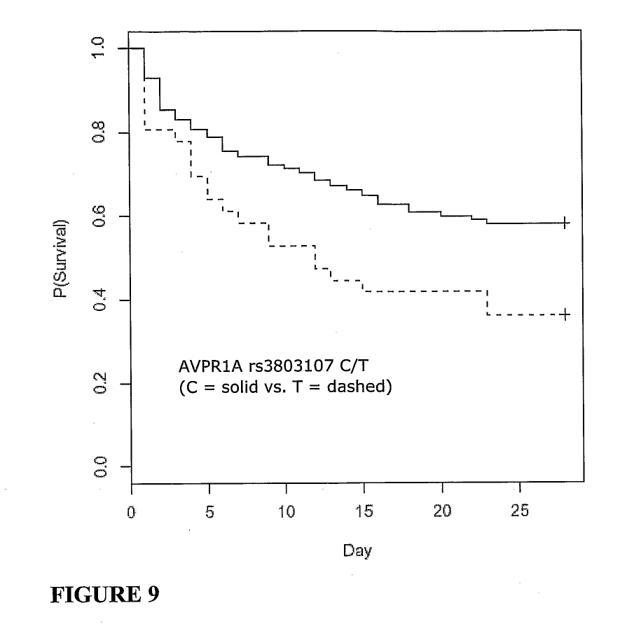

FIG. 9 and TABLE 4.34 summarize important SNP-phenotype association results for AVPR1A rs3803107. Subjects with the C allele had a significantly increased 28-day survival (P=0.0377) and significantly more days alive (P=0.0206) and significantly more days alive and free of vasopressors (P=0.0386), more days alive and free of vasopressors at doses of 2 ug/min (P=0.02=286), 5 (P=0.0296) and 15 ug/min (P=0.0132), inotropes (P=0.0379), 4 of 4 SIRS criteria (P=0.0494), cardiovascular dysfunction (P=0.0365), respiratory dysfunction (P=0.0214) mechanical ventilation (P=0.0411), neurological dysfunction (P=0.0488) and INR>1.5 (P=0.0296). Subjects with the AVPR1A rs3803107 C allele also had a strong trend for more days alive and free of any hepatic dysfunction (P=0.0894). These findings indicate that, Asian subjects with SIRS who had the C allele at AVPR1A rs3803107 have less need of vasopressors and are at decreased risk of SIRS and organ dysfunction (cardiovascular, respiratory, neurological and hepatic).

TABLE 4.34
Days alive and free of organ dysfunction (DAF) by allele of arginine vasopressin receptor 1a
(AVPR1A) rs3803107 (C vs. T) in a cohort of Asian subjects with systematic inflammatory
response syndrome. For all variables besides 28-day survival, data is given as 25^{th} percentile/
median/75^{th} percentile. For 28-day survival, data is given as % (N survived/N total).

| | C | T | Combined | Test |
|---|---|---|---|---|
| | (N = 186) | (N = 30) | (N = 216) | Statistic |
| SURVIVAL | 60% | 40% (12/30) | 57% | X\hat{ }2 = 4.32 d.f. = 1 P = 0.0377 |
| | (112/186) | | (124/216) | |
| DA | 7/28/28 | 3.25/10.5/28 | 6/28/28 | F = 5.44 d.f. = 1.214 P = 0.0206 |
| PRESS.DAF | 4/24/28 | 1/8/26 | 2/21.5/28 | F = 4.33 d.f. = 1.214 P = 0.0386 |
| PRESS2.DAF | 4/24.50/28 | 1/8/26 | 2.75/21.50/ | F = 4.33 d.f. = 1.214 P = 0.0386 |
| | | | 28 | |
| PRESS5.DAF | 5/25/28 | 1/8/26.75 | 3.75/23.50/ | F = 4.8 d.f. = 1.214 P = 0.0296 |
| | | | 28 | |
| PRESS15.DAF | 6/27/28 | 1/8/27 | 4.75/26/28 | F = 6.25 d.f. = 1.214 P = 0.0132 |
| INO.DAF | 6/28/28 | 2.25/9/28 | 4.75/27.50/ | F = 4.36 d.f. = 1.214 P = 0.0379 |
| | | | 28 | |
| MSIRS2.DAF | 0/3.5/21 | 0/0/3 | 0/3/20 | F = 9.25 d.f. = 1.214 P = 0.00265 |
| MSIRS3.DAF | 1/17/27 | 0/1.5/21.5 | 1/14.5/26 | F = 7.43 d.f. = 1.214 P = 0.00693 |
| MSIRS4.DAF | 5/25/28 | 2/7/27.8 | 4/25/28 | F = 3.66 d.f. = 1.214 P = 0.057 |
| CSIRS2.DAF | 0/4/23 | 0/0/6 | 0/3/21.3 | F = 9.13 d.f. = 1.214 P = 0.00282 |
| CSIRS3.DAF | 2/17/27 | 0/2/19.3 | 1/16/26 | F = 8.5 d.f. = 1.214 P = 0.00394 |
| CSIRS4.DAF | 5/25/28 | 2.25/7.50/ | 4.75/25/28 | F = 3.91 d.f. = 1.214 P = 0.0494 |
| | | 27.75 | | |
| CVS.DAF | 1/13.5/27 | 0/4/17 | 1/11/26 | F = 4.43 d.f. = 1.214 P = 0.0365 |
| RESP.DAF | 0/15/27 | 0/1.5/23.5 | 0/10/27 | F = 5.37 d.f. = 1.214 P = 0.0214 |
| VENT.DAF | 0/10/26 | 0/0.5/19 | 0/8.5/26 | F = 4.22 d.f. = 1.214 P = 0.0411 |
| CNS.DAF | 5/27/28 | 1/7/28 | 3/24/28 | F = 3.93 d.f. = 1.214 P = 0.0488 |
| INR.DAF | 5/27/28 | 1/7.5/28 | 4/25/28 | F = 4.79 d.f. = 1.214 P = 0.0296 |
| ANYHEP.DAF | 4.25/27/28 | 1/8.5/28 | 2/20/28 | F = 2.91 d.f. = 1.214 P = 0.0894 |

N, number of subjects.

2.3.3 AVPR1A rs10877970

2.3.3.1 Systematic Inflammatory Response Syndrome—Caucasians

TABLE 4.35 gives the baseline characteristics (age, gender, APACHE II score, medical vs. surgical diagnosis, sepsis upon admission, sepsis anytime, septic shock upon admission and septic shock anytime) of 725 Caucasian SIRS subjects who were successfully genotyped (CC vs. TT/CT) at AVPR1A rs10877970. No significant differences were detected between the two genotype groups on admission to the ICU.

TABLE 4.35
Baseline characteristics of a cohort of Caucasian Subjects with systematic inflammatory response
syndrome by genotype of arginine vasopressin receptor 1a (AVPR1A) rs10877970 (CC vs.
TT/CT). For age and APACHE II score, data is given as 25^{th} percentile/median/75^{th} percentile
For all other variables, data is given as % (N survived/N total).

| | CC | TT/CT | Combined | Test |
|---|---|---|---|---|
| | (N = 20) | (N = 705) | (N = 725) | Statistic |
| AGE | 49.5/56/68.5 | 44/58/71 | 46/59/71 | F = 0.1 d.f. = 1.723 P = 0.75 |
| GENDER | 70% (14/20) | 61% (432/705) | 62% (446/725) | X\hat{ }2 = 0.63 d.f. = 1 P = 0.429 |
| APACHEII | 22/25.5/27.3 | 16/22/27 | 16/21.5/27 | F = 3.25 d.f. = 1.723 P = 0.0716 |
| SURGICAL | 15% (3/20) | 22% (158/705) | 22% (161/725) | X\hat{ }2 = 0.62 d.f. = 1 P = 0.432 |
| SEP.ADMIT | 80% (16/20) | 78% (552/705) | 78% (568/725) | X\hat{ }2 = 0.03 d.f. = 1 P = 0.855 |
| SEP.ANY | 80% (16/20) | 80% (565/705) | 80% (581/725) | X\hat{ }2 = 0 d.f. = 1 P = 0.987 |
| SS.ADMIT | 60% (12/20) | 51% (357/705) | 51% (369/725) | X\hat{ }2 = 0.68 d.f. = 1 P = 0.409 |
| SS.ANY | 60% (12/20) | 54% (383/705) | 54% (395/725) | X\hat{ }2 = 0.25 d.f. = 1 P = 0.615 |

N, number of subjects.

TABLE 4.36 summarizes important SNP-phenotype associations for AVPR1A rs10877970. Subjects with either the TT or CT genotype had significantly more days alive and free of acute lung injury (P=0.0331), respiratory dysfunction (P=0.0134) and mechanical ventilation (P=0.0276). Subjects with either the AVPR1A rs10877970 TT or CT genotype also had a strong trend for more days alive and free of vasopressors (P=0.0183), and more days alive and free of vasopressors at doses of 2 ug/min (P=0.0638) and 5 ug/min (0.0575). These findings indicate that Caucasian subjects with SIRS with the TT or CT genotype at AVPR1A rs10877970 have less need of vasopressors, are at decreased risk of acute lung injury and organ dysfunction (respiratory).

TABLE 4.36
Days alive and free of organ dysfunction (DAF) by
genotype of arginine vasopressin receptor 1a (AVPR1A) rs10877970 (CC vs. TT/CT)
in a cohort of Caucasian subjects with systematic inflammatory response syndrome.
Data is given as 25^{th} percentile/median/75^{th} percentile.

| | CC | TT/CT | Combined | Test |
|---|---|---|---|---|
| | (N = 20) | (N = 705) | (N = 725) | Statistic |
| ALI.DAF | 2/9/24 | 5/24/28 | 4/24/28 | F = 4.56 d.f. = 1.723 P = 0.0331 |
| PRESS.DAF | 6/21/25.3 | 10/26/ | 9/26/28 | F = 3.45 d.f. = 1.723 P = 0.0638 |
| | | 28 | | |
| PRESS2.DAF | 6/21/26 | 10/26/ | 10/26/ | F = 3.31 d.f. = 1.723 P = 0.0692 |
| | | 28 | 28 | |
| PRESS5.DAF | 6.5/23/26 | 11/26/ | 10/26/ | F = 3.62 d.f. = 1.723 P = 0.0575 |
| | | 28 | 28 | |
| CVS.DAF | 3.25/14/ | 5/23/27 | 5/23/27 | F = 2.68 d.f. = 1.723 P = 0.102 |
| | 24.25 | | | |
| RESP.DAF | 0/5.5/20 | 1/21/27 | 1/20/26 | F = 6.15 d.f. = 1.723 P = 0.0134 |
| PF300.DAF | 0/0/2.75 | 0/1/11 | 0/1/10 | F = 3.4 d.f. = 1.723 P = 0.0656 |
| VENT.DAF | 0/5.5/20 | 0/19/26 | 0/19/26 | F = 4.87 d.f. = 1.723 P = 0.0276 |
| AFFD.DAF | 0/0/0 | 0/0/4 | 0/0/3 | F = 3.12 d.f. = 1.723 P = 0.0779 |

N, number of subjects.

2.3.3.2 Systematic Inflammatory Response Syndrome—Asians

TABLE 4.37 gives the baseline characteristics (age, gender, APACHE II score, medical vs. surgical diagnosis, sepsis upon admission, sepsis anytime, septic shock upon admission and septic shock anytime) of the 108 Asian systematic inflammatory response syndrome subjects who were successfully genotyped (C vs. T) at AVPR1A rs10877970. No significant differences, other than a small difference in APACHE II score, were detected between the two allelic groups on admission to the ICU.

TABLE 4.37
Baseline characteristics of a cohort of Asian Subjects with systematic inflammatory response
syndrome by allele of arginine vasopressin receptor 1a (AVPR1A) rs10877970 (C vs. T).
For age and APACHE II score, data is given as 25^{th} percentile/median/75^{th} percentile.
For all other variables, data is given as % (N survived/N total).

| | C | T | Combined | Test |
|---|---|---|---|---|
| | (N = 33) | (N = 183) | (N = 216) | Statistic |
| AGE | 57/73/77.0 | 51/68/77 | 54.5/69/76 | F = 0.52 d.f. = 1.214 P = 0.471 |
| GENDER | 48% (16/33) | 60% (110/183) | 58% (126/216) | X\hat{ }2 = 1.55 d.f. = 1 P = 0.212 |
| APACHEII | 19/26/34 | 17/23/29 | 17/23/30 | F = 4 d.f. = 1.214 P = 0.0467 |
| SURGICAL | 18% (6/33) | 24% (44/183) | 23% (50/216) | X\hat{ }2 = 0.54 d.f. = 1 P = 0.462 |
| SEP.ADMIT | 76% (25/33) | 76% (139/183) | 76% (164/216) | X\hat{ }2 = 0 d.f. = 1 P = 0.98 |
| SEP.ANY | 79% (26/33) | 79% (144/183) | 79% (170/216) | X\hat{ }2 = 0 d.f. = 1 P = 0.99 |
| SS.ADMIT | 52% (17/33) | 54% (99/183) | 54% (116/216) | X\hat{ }2 = 0.08 d.f. = 1 P = 0.784 |
| SS.ANY | 58% (19/33) | 63% (115/183) | 62% (134/216) | X\hat{ }2 = 0.33 d.f. = 1 P = 0.566 |

N, number of subjects.

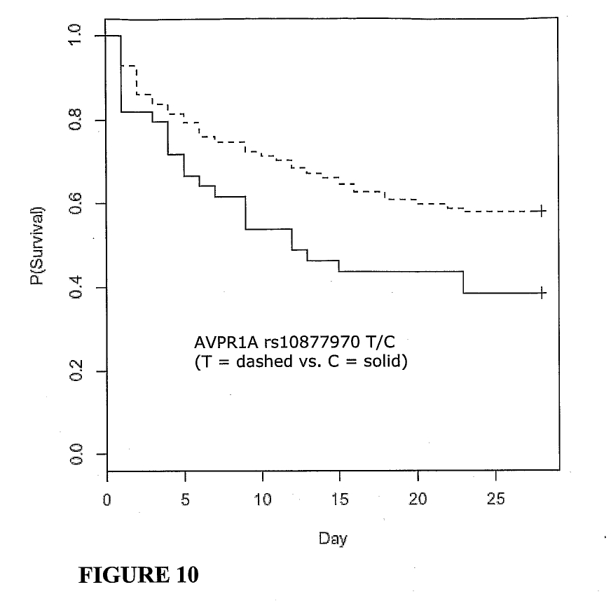

FIG. 10 and TABLE 4.38 summarizes important SNP-phenotype association results for AVPR1A rs10877970. Subjects with the AVPR1A rs10877970 T allele had a strong trend for increased 28-day survival (P=0.0586) and significantly more days alive (P=0.0349) and significantly more days alive and free of vasopressors at doses of 5 ug/min (P=0.0417) and 15 ug/min (P=0.0175), inotropes (P=0.0423) and respiratory dysfunction (P=0.0427). Subjects with the AVPR1A rs10877970 T allele also showed a strong trend for more days alive and free of 4 of 4 SIRS criteria (P=0.0655) cardiovascular dysfunction (P=0.079), ventilation (P=0.057), neurological dysfunction (P=0.064) and any hepatic dysfunction (P=0.0827). These findings indicate that Asian subjects with SIRS who had the T allele at AVPR1A rs10877970 have less need of vasopressors and are at a decreased risk of severe SIRS and organ dysfunction (cardiovascular, respiratory, neurological and hepatic).

TABLE 4.38
Days alive and free of organ dysfunction (DAF)
by allele of arginine vasopressin receptor 1a (AVPR1A) rs10877970 (C vs. T)
in a cohort of Asian subjects with systematic inflammatory response
syndrome. For all variables besides 28-day survival,
data is given as 25^{th} percentile/ median/75^{th} percentile.
For 28-day survival, data is given as % (N survived/N total).

| | C | T | Combined | Test |
|---|---|---|---|---|
| | (N = 33) | (N = 183) | (N = 216) | Statistic |
| SURVIVAL | 42% (14/33) | 60% | 57% | X\hat{ }2 = 3.58 d.f. = 1 P = 0.0586 |
| | | (110/183) | (124/216) | |
| DA | 4/12/28 | 7/28/28 | 6/28/28 | F = 4.51 d.f. = 1.214 P = 0.0349 |
| PRESS.DAF | 1/9/26 | 4/23/28 | 2/21.5/28 | F = 3.72 d.f. = 1.214 P = 0.0551 |
| PRESS2.DAF | 1/9/26 | 4/24/28 | 2.75/21.5/ | F = 3.72 d.f. = 1.214 P = 0.0551 |
| | | | 28 | |
| PRESS5.DAF | 1/9/27 | 5/25/28 | 3.75/23.5/ | F = 4.2 d.f. = 1.214 P = 0.0417 |
| | | | 28 | |
| PRESS15.DAF | 1/9/27 | 6/27/28 | 4.75/26/28 | F = 5.73 d.f. = 1.214 P = 0.0175 |
| INO.DAF | 3/9/28 | 6/28/28 | 4.75/27.5/ | F = 4.17 d.f. = 1.214 P = 0.0423 |
| | | | 28 | |
| MSIRS2.DAF | 0/0/7 | 0/3/21.5 | 0/3/20 | F = 8.5 d.f. = 1.214 P = 0.00393 |
| MSIRS3.DAF | 0/2/22 | 1/16/27 | 1/14.5/26 | F = 6.29 d.f. = 1.214 P = 0.0129 |
| MSIRS4.DAF | 2/7/28 | 5/25/28 | 4/25/28 | F = 3.19 d.f. = 1.214 P = 0.0757 |
| CSIRS2.DAF | 0/0/8 | 0/5/23 | 0/3/21.3 | F = 7.82 d.f. = 1.214 P = 0.00565 |
| CSIRS3.DAF | 0/3/20 | 2/18/27 | 1/16/26 | F = 7.12 d.f. = 1.214 P = 0.00819 |
| CSIRS4.DAF | 3/8/28 | 5/25/28 | 4.75/25/28 | F = 3.43 d.f. = 1.214 P = 0.0655 |
| CVS.DAF | 0/5/21 | 1/13/27 | 1/11/26 | F = 3.11 d.f. = 1.214 P = 0.079 |
| RESP.DAF | 0/5/24 | 0/14/27 | 0/10/27 | F = 4.16 d.f. = 1.214 P = 0.0427 |
| VENT.DAF | 0/1/19 | 0/10/26 | 0/8.5/26 | F = 3.66 d.f. = 1.214 P = 0.057 |
| CNS.DAF | 1/9/28 | 5/27/28 | 3/24/28 | F = 3.47 d.f. = 1.214 P = 0.064 |
| INR.DAF | 1/9/28 | 5/27/28 | 4/25/28 | F = 3.67 d.f. = 1.214 P = 0.0568 |
| ANYHEP.DAF | 1/9/28 | 4.5/28/28 | 2/20/28 | F = 3.04 d.f. = 1.214 P = 0.0827 |

N, number of subjects.

### Example 3

### Increased Use of Vasopressin

### Methods

### Cohort Selection

To investigate whether genotype predicts increased use of vasopressin, a subset of Caucasian subjects with septic shock was selected for this analysis (N=543).

### Data Analysis

All data analysis was carried out using statistical packages available in R(R Core Development Group, 2005-R Development Core Team (www.R-project.org). R: A language and environment for statistical computing. Vienna, Austria. 2005). Chi-square tests were used to identify significant associations between SNP and increased use of vasopressin as well as to identify baseline characteristics (age, gender, admitting APACHE II score, and medical vs. surgical admitting diagnosis) requiring post-hoc, multivariate adjustment.

### Results

### 3.1 Leucyl/Cystinyl Aminopeptidase (LNPEP)

3.1.1 Association of CC genotype of LNPEP rs18059 with Use of Vasopressin

It was unknown whether SNPs within the LNPEP gene and those regions immediately upstream and downstream are associated with the use of vasopressin. It was found that LNPEP rs18059 is associated with the use of vasopressin by comparing LNPEP rs18059 genotypes for vasopressin-treated subjects (N=73) with control subjects who did not receive vasopressin at any time during their ICU stay (N=366). Baseline characteristics for septic shock subjects with LNPEP rs18059 genotypes are shown in Table 5.1. No significant differences between the genotype groups were detected on admission to the ICU.

TABLE 5.1
Baseline characteristics of Caucasian ICU septic-shock subjects by leucyl/cystinyl aminopeptidase
(LNPEP) rs18059 genotype. For age and APACHE II score, data is given as 25^{th} percentile|
median|75^{th} percentile. For all other variables, data is given as % (N/N total).

| | CC | CT | TT | Combined | Test |
|---|---|---|---|---|---|
| | (N = 108) | (N = 231) | (N = 100) | (N = 439) | Statistic |
| AGE | 46\|59\|71 | 48\|63\|72 | 48.75\|62.5\|72 | 48\|61\|72 | F = 1.1 d.f. = 2.436 P = 0.334 |
| GENDER | 65% (70/108) | 64% (148/231) | 62% (62/100) | 64% (280/439) | X\hat{ }2 = 0.2 d.f. = 2 P = 0.907 |
| APACHE II | 19\|25\|32 | 20.5\|26\|31 | 21\|25\|30 | 20\|26\|31 | F = 0.39 d.f. = 2.436 P = 0.679 |
| SURGICAL | 28% (30/108) | 29% (66/231) | 25% (25/100) | 28% (121/439) | X\hat{ }2 = 0.45 d.f. = 2 P = 0.799 |

N, number of subjects.

Table 5.2 shows the distribution of vasopressin administration by LNPEP rs18059 genotype. Subjects with the LNPEP rs18059 CC genotype were observed to have been administered vasopressin more frequently than controls compared with subjects who were LNPEP rs18059 CT or TT (P=0.0257)

TABLE 5.2
Measure of vasopressin treatment of
Caucasian ICU septic shock subjects by genotype of
leucyl/cystinyl aminopeptidase (LNPEP) rs18059.

| | No | Vasopressin- | | |
|---|---|---|---|---|
| | Vasopressin | treated | Combined | Test |
| | (N = 366) | (N = 73) | (N = 439) | Statistic |
| CC | 22% (81/366) | 37% (27/73) | 25% | X\hat{ }2 = 7.32 d.f. = 2 |
| | | | (108/439) | P = 0.0257 |
| CT | 54% (198/366) | 45% (33/73) | 53% | |
| | | | (231/439 | |
| TT | 24% (87/366) | 18% (13/73) | 23% | |
| | | | (100/439) | |

3.1.2 Association of AA genotype of LNPEP rs27711 with Use of Vasopressin

It was unknown whether SNPs within the LNPEP gene and those regions immediately upstream and downstream are associated with the use of vasopressin. It was found that LNPEP rs27711 is associated with the use of vasopressin by comparing the frequency of LNPEP rs27711 genotypes for vasopressin-treated subjects (N=70) and control subjects who did not receive vasopressin at any time during their ICU stay (N=368). Baseline characteristics for septic shock subjects with LNPEP rs27711 genotypes are shown in Table 5.3. No significant differences between the genotype groups were detected on admission to the ICU.

TABLE 5.3
Baseline characteristics of Caucasian ICU septic shock subjects by leucyl/cystinyl
aminopeptidase (LNPEP) rs27711 genotype. For age and APACHE II score,
data is given as 25^{th} percentile| median|75^{th} percentile.
For all other variables, data is given as % (N/N total).

| | AA | AG | GG | Combined | Test |
|---|---|---|---|---|---|
| | (N = 72) | (N = 223) | (N = 143) | (N = 438) | Statistic |
| AGE | 44.5\|58.5\|71 | 48\|63\|72 | 49\|63\|72 | 48\|61\|72 | F = 0.78 d.f. = 2.435 |
| | | | | | P = 0.46 |
| GENDER | 65% (47/72) | 64% | 63% (90/143) | 64% (279/438) | X\hat{ }2 = 0.11 d.f. = 2 |
| | | (142/223) | | | P = 0.945 |
| APACHE II | 19\|25.5\|33 | 20.5\|26\|30 | 21\|26\|30 | 20\|26\|31 | F = 0.16 d.f. = 2.435 |
| | | | | | P = 0.854 |
| SURGICAL | 28% (20/72) | 29% | 22% (32/143) | 26% (116/438) | X\hat{ }2 = 1.86 d.f. = 2 |
| | | (64/223) | | | P = 0.394 |

N, number of subjects.

Table 5.4 shows the distribution of vasopressin administration by LNPEP rs27711 genotype. Subjects with the LNPEP rs27711 AA genotype were more frequently observed to be administered vasopressin compared to subjects with LNPEP rs27711 AG or GG genotypes (P=0.0033).

TABLE 5.4
Measure of vasopressin treatment of Caucasian ICU septic shock subjects
by genotype of leucyl/cystinyl aminopeptidase (LNPEP) rs27711.

| | No | Vasopressin- | | |
|---|---|---|---|---|
| | Vasopressin | treated | Combined | Test |
| | (N = 368) | (N = 70) | (N = 438) | Statistic |
| AA | 14% (51/368) | 30% (21/70) | 16% (72/438) | X\hat{ }2 = 11.45 |
| | | | | d.f. = 2 |
| | | | | P = 0.0033 |
| AG | 53% (195/368) | 40% (28/70) | 51% (223/438) | |
| GG | 33% (122/368) | 30% (21/70) | 33% (143/438) | |

3.1.3 Association of GG genotype of LNPEP rs10051637 with Use of Vasopressin

It was unknown whether SNPs within the LNPEP gene and those regions immediately upstream and downstream are associated with the use of vasopressin. It was found that LNPEP rs10051637 is associated with the use of vasopressin by comparing the frequency of LNPEP rs10051637 genotypes for vasopressin-treated subjects (N=72) with control subjects (N=36I) who did not receive vasopressin at any time during their ICU stay. Baseline characteristics for septic shock subjects with LNPEP rs10051637 genotypes are shown in Table 5.5. No significant differences between the genotype groups were detected on admission to the ICU.

TABLE 5.5
Baseline characteristics of Caucasian ICU septic shock subjects by leucyl/cystinyl
aminopeptidase (LNPEP) rs10051637 genotype. For age and APACHE II score,
data is given as 25^{th} percentile| median|75^{th} percentile.
For all other variables, data is given as % (N/N total).

| | AA | AG | GG | Combined | Test |
|---|---|---|---|---|---|
| | (N = 133) | (N = 223) | (N = 77) | (N = 433) | Statistic |
| AGE | 49\|63\| | 48\|63\|72 | 43\|58\|71 | 48\|61\|72 | F = 2.05 d.f. = 2,430 P = 0.130 |
| | 72 | | | | |
| GENDER | 62% | 66% | 66% | 65% | X\hat{ }2 = 0.76 d.f. = 2 P = 0.682 |
| | (82/133) | (147/223) | (51/77) | (280/433) | |
| APACHE II | 21\|26\| | 21\|26\|30 | 19\|25\|33 | 20\|26\|31 | F = 0.04 d.f. = 2,430 P = 0.96 |
| | 31 | | | | |
| SURGICAL | 23% | 29% | 29% | 27% | X\hat{ }2 = 1.75 d.f. = 2 P = 0.416 |
| | (30/133) | (64/223) | (22/77) | (116/433) | |

N, number of subjects.

Table 5.4 shows the distribution of vasopressin administration by LNPEP rs10051637 genotype. Subjects with the GG genotype of LNPEP rs10051637 were more frequently observed to be administered vasopressin (P<0.001) compared to subjects who carried the AG or AA genotype of LNPEP rs10051637 (TABLE 5.6).

TABLE 5.6
Measure of vasopressin treatment of Caucasian ICU septic shock subjects
by genotype of leucyl/cystinyl aminopeptidase (LNPEP) rs10051637.

| | No | Vasopressin- | | |
|---|---|---|---|---|
| | Vasopressin | treated | Combined | Test |
| | (N = 361) | (N = 72) | (N = 433) | Statistic |
| AA | 32% (114) | 26% (19) | 31% (133) | X\hat{ }2 = 14.38 |
| | | | | d.f. = 2 |
| | | | | P < 0.001 |
| AG | 54% (194) | 40% (29) | 52% (223) | |
| GG | 15% (53) | 33% (24) | 18% (77) | |

### 3.2 Arginine Vasopressin Receptor 1a (AVPR1A)

3.2.1 Association of CT genotype of AVPR1A rs1495027 with Use of Vasopressin

It was unknown whether SNPs within the AVPR1A gene and those regions immediately upstream and downstream are associated with the use of vasopressin in subjects with septic shock. It was found that AVPR1A rs1495027 is associated with the use of vasopressin by comparing the frequency of AVPR1A rs1495027 genotypes for vasopressin-treated subjects (N=72) with control subjects (N=361) who did not receive vasopressin at any time during their ICU stay.

Baseline characteristics for septic shock subjects with AVPR1A rs1495027 genotypes are shown in Table 5.7. No significant differences between the genotype groups were detected on admission to the ICU.

TABLE 5.7
Baseline characteristics of Caucasian ICU septic shock subjects by genotype of arginine
vasopressin receptor 1a (AVPR1A) rs1495027. For age and APACHE II score, data is given as
25^{th} percentile|median|75^{th} percentile. For all other variables, data is given as % (N/N total).

| | CC | CT | TT | Combined | Test |
|---|---|---|---|---|---|
| | (N = 143) | (N = 218) | (N = 72) | (N = 433) | Statistic |
| AGE | 48\|61\|72 | 46\|60\|71.75 | 51\|63.50\|73 | 48\|61\|72 | F = 0.84 d.f. = 2,430 P = 0.433 |
| GENDER | 61% (87/143) | 69% (150/218) | 58% (42/72) | 64% (279/433) | X\hat{ }2 = 3.8 d.f. = 2 P = 0.15 |
| APACHE II | 19.5\|26\|31 | 20\|25\|31 | 21.75\|26\|30.75 | 20\|26\|31 | F = 0.62 d.f. = 2,430 P = 0.536 |
| SURGICAL | 24% (35/143) | 28% (62/218) | 26% (19/72) | 27% (116/433) | X\hat{ }2 = 0.7 d.f. = 2 P = 0.705 |

N, number of subjects.

Table 5.4 shows the distribution of vasopressin administration by AVPR1A rs1495027 genotype. Subjects with the AVPR1A rs1495027 CT genotype had significantly increased use of vasopressin (P=0.0240) compared to subjects who carried either the CC or TT genotype of AVPR1A T AVPR1A rs1495027 (TABLE 5.8).

TABLE 5.8
Measure of vasopressin treatment of Caucasian ICU
septic shock subjects by genotype of vasopressin
receptor 1a (AVPR1A) rs1495027.

| | | Vasopressin- | | |
|---|---|---|---|---|
| | No Vasopressin | treated | Combined | Test |
| | (N = 361) | (N = 72) | (N = 433) | Statistic |
| CC | 36% (129) | 19% (14) | 33% (143) | X\hat{ }2 = 7.46 d.f. = 2 |
| | | | | P = 0.0240 |
| CT | 48% (173) | 62% (45) | 50% (218) | |
| TT | 16% (59) | 18% (13) | 17% (72) | |

### Example 4

### Biological Plausibility

Examples 1-3 show that polymorphisms of the AVP, AVPR1A and LNPEP genes are associated with altered outcome in critically ill subjects. To further explore the relationship between inflammation and infection, the present example examines subjects with non-septic causes of systemic inflammatory response syndrome by analyzing SNP-phenotype interactions in subjects having undergone cardiopulmonary bypass surgery. If an AVP. AVPR1A. LNPEP or LRAP gene polymorphism was associated with altered survival and organ dysfunction, that polymorphism is also likely to be associated with changes in pro-inflammatory proteins such as serum granulocyte colony stimulating factor (GCSF), interleukin 8 (IL-8) and monocyte chemotactic protein 1 (MCP1).

### Methods

### Cohort Selection

The Biological Plausibility cohort was used for this study.

### Measurement of Chemokine and Cytokines

After induction of anesthesia and placement of systemic and pulmonary artery catheters that were routinely inserted for clinical purposes at SPH, blood was obtained at baseline and at 3 hours post-operatively for serum. GCSF. MCP1 and IL-8 measurements were made using ELISA.

### Data Analysis

The primary outcome variables for the Biological Plausibility cohort were change in GCSF, MCP1 and IL-8 concentrations from baseline to three hours after surgery. All data analysis was carried out using statistical packages available in R(R Core Development Group, 2005-R Development Core Team (www.R-project.org). Vienna Austria 200). Chi-squared and Kruskal-Wallis test statistics were used to identify significant SNP-phenotype and associations, as well as to look at baseline characteristics.

### Results

### 4.1 Leucyl/Cystinyl Aminopeptidase (LNPEP)

4.1.1 LNPEP rs18059

TABLE 6.1 summarizes the baseline characteristics of 69 non-septic SIRS subjects who were successfully genotyped (LNPEP rs18059 CC (N=20) vs CT (N=36) vs TT (N=13)) at LNPEP rs18059. No significant differences were detected between the three genotype groups on admission to the CSICU.

TABLE 6.1
Baseline characteristics of a cohort of non-septic CSICU subjects diagnosed with systematic
inflammatory response syndrome by genotype of leucyl/cystinyl aminopeptidase (LNPEP)
rs18059 (CC vs. CT vs TT).

| | CC | CT | TT | Combined | Test |
|---|---|---|---|---|---|
| | (N = 20) | (N = 36) | (N = 13) | (N = 69) | Statistic |
| AGE | 59.25\|64.50\| | 61.00\|65.00\| | 60.00\|66.00\| | 58.25\|65.50\| | F = 0.15 d.f. = 2.66 |
| | 73.25 | 70.25 | 72.00 | 70.75 | P = 0.865 |
| GENDER | 70% (14) | 61% (22) | 77% (10) | 67% (46) | X\hat{ }2 = 1.22 d.f. = 2 |
| | | | | | P = 0.545 |
| SMOKER | 25% (5) | 19% (7) | 38% (5) | 25% (17) | X\hat{ }2 = 1.86 d.f. = 2 |
| | | | | | P = 0.394 |
| DIABETES | 15% (3) | 22% (8) | 23% (3) | 20% (14) | X\hat{ }2 = 0.49 d.f. = 2 |
| | | | | | P = 0.782 |
| H. TENSE | 60% (12) | 56% (20) | 46% (6) | 55% (38) | X\hat{ }2 = 0.62 d.f. = 2 |
| | | | | | P = 0.734 |
| EJEC.FRAC | 0.37\|0.50\| | 0.50\|0.50\| | 0.46\|0.58\|0.60 | 0.50\|0.50\|0.60 | F = 0.56 d.f. = 2.64 |
| | 0.60 | 0.60 | | | P = 0.575 |
| BYPASS | 1.48\|1.65\| | 1.13\|1.57\| | 1.33\|1.73\|2.45 | 1.31\|1.65\|2.05 | F = 0.56 d.f. = 2.66 |
| | 2.02 | 2.00 | | | P = 0.575 |
| CLAMP | 1.04\|1.32\| | 0.83\|1.19\| | 0.93\|1.43\|1.78 | 0.92\|1.29\|1.70 | F = 0.2 d.f. = 2.66 |
| | 1.57 | 1.69 | | | P = 0.822 |
| APROTININ | 5% (1) | 8% (3) | 8% (1) | 7% (5) | X\hat{ }2 = 0.22 d.f. = 2 |
| | | | | | P = 0.897 |

TABLE 6.2 summarizes important SNP-biomarker associations. Subjects with the CC genotype had significantly smaller increase in serum GCSF levels (P=0.0135) post-cardiopulmonary bypass surgery. These findings suggest that non-septic SIRS Subjects with the CC genotype at LNPEP rs18059 are more likely to experience a less intense chemokine (GCSF) response after cardiopulmonary bypass surgery.

TABLE 6.2
Biological plausibility of leucyl/cystinyl aminopeptidase association using biomarkers in a cohort
of non-septic CSICU subjects diagnosed with systematic inflammatory response syndrome by
genotype of leucyl/cystinyl aminopeptidase (LNPEP) rs18059. Biomarkers are measured in pg/ml.

| | | | | Combined | |
|---|---|---|---|---|---|
| | CC (N = 20) | CT (N = 36) | TT (N = 13) | (N = 69) | Test Statistic |
| GCSF.3 | 123/183/276 | 219/292/497 | 236/287/344 | 179/260/368 | F = 5.26 d.f. = 2.66 |
| | | | | | P = 0.00758 |
| GCSF.DIF | 108/164/266 | 199/287/492 | 210/264/330 | 161/249/365 | F = 4.6 d.f. = 2.66 |
| | | | | | P = 0.0135 |
| MCP1.0 | 125.2/186.6/211.3 | 165.0/195.3/281.2 | 95.7/138.1/226.7 | 134.9/182.0/245.2 | F = 2.54 d.f. = 2.66 |
| | | | | | P = 0.0862 |

4.1.2 LNPEP rs27711

TABLE 6.3 summarizes the baseline characteristics of 69 non-septic SIRS subjects who were successfully genotyped (AA (N=14) vs. AG/GG (N=55)) at LNPEP rs27711. No significant differences between the genotype groups were detected on admission to the CSICU.

TABLE 6.3
Baseline characteristics of a cohort of non-septic CSICU subjects diagnosed with systematic
inflammatory response syndrome by genotype of leucyl/cystinyl aminopeptidase (LNPEP)
rs27711 (AA vs. GG/AG).

| | AA | GG/AG | Combined | Test |
|---|---|---|---|---|
| | (N = 14) | (N = 55) | (N = 69) | Statistic |
| AGE | 60.25/63.00/69.25 | 60.50/66.00/71.50 | 58.25/65.50/70.75 | F = 0.52 d.f. = 1.67 |
| | | | | P = 0.473 |
| GENDER | 64% (9) | 69% (38) | 68% (47) | X\hat{ }2 = 0.12 d.f. = 1 |
| | | | | P = 0.73 |
| SMOKER | 29% (4) | 24% (13) | 25% (17) | X\hat{ }2 = 0.15 d.f. = 1 |
| | | | | P = 0.702 |
| DIABETES | 14% (2) | 20% (11) | 19% (13) | X\hat{ }2 = 0.24 d.f. = 1 |
| | | | | P = 0.625 |
| H.TENSE | 64% (9) | 55% (30) | 57% (39) | X\hat{ }2 = 0.43 d.f. = 1 |
| | | | | P = 0.512 |
| EJEC.FRAC | 0.35/0.50/0.60 | 0.50/0.50/0.60 | 0.50/0.50/0.60 | F = 0.37 d.f. = 1.65 |
| | | | | P = 0.544 |
| BYPASS | 1.51225/1.63350/ | 1.25850/1.65000/2.08300 | 1.31700/1.65000/2.05000 | F = 0.44 d.f. = 1.67 |
| | 2.06225 | | | P = 0.511 |
| CLAMP | 1.07900/1.33300/ | 0.85850/1.21700/1.67500 | 0.92475/1.29150/1.70000 | F = 0.44 d.f. = 1.67 |
| | 1.61225 | | | P = 0.511 |
| APROTININ | 7% (1) | 7% (4) | 7% (5) | X\hat{ }2 = 0 d.f. = 1 P = 0.987 |

TABLE 6.4 summarizes important SNP-biomarker associations observed for LNPEP rs27711. Subjects with the LNPEP rs27711 AA genotype showed a smaller change in GCSF levels from baseline to 3 hours post-surgery (P<0.001) and had lower preoperative interleukin 8 (IL8) levels (P=0.05) than subjects with LNPEP rs27711 AG or GG genotypes. These findings suggest that non-septic SIRS Subjects with the AA genotype at LNPEP rs27711 are more likely to experience a less intense chemokine (GCSF) response after cardiopulmonary bypass and are more likely to have higher baseline levels of IL-8.

TABLE 6.4
Biological plausibility of leucyl/cystinyl aminopeptidase association
using biomarkers in a cohort of non-septic CSICU subjects
diagnosed with systematic inflammatory response syndrome by
genotype of leucyl/cystinyl aminopeptidase rs27711 (AA vs. GG/AG).

| | | GG/AG | Combined | |
|---|---|---|---|---|
| | AA (N = 14) | (N = 55) | (N = 69) | Test Statistic |
| GCSF.3 | 115/145/209 | 221/287/442 | 179/260/ | F = 15.4 d.f. = 1.67 |
| | | | 368 | P < 0.001 |
| GCSF.DIF | 103/138/181 | 205/274/431 | 161/249/ | F = 14.3 d.f. = 1.67 |
| | | | 365 | P < 0.001 |
| IL8.0 | 0.0/0.0/12.8 | 0.0/13.4/21.1 | 0.0/7.2/ | F = 3.89 d.f. = 1.67 |
| | | | 20.2 | P = 0.0528 |

4.1.3 LNPEP rs10051637

TABLE 6.5 summarizes the baseline characteristics of 70 non-septic SIRS subjects who were successfully genotyped (AA/AG vs. GG) al LNPEP rs10051637. No significant differences between the genotype groups were detected on admission to the CSICU.

TABLE 6.5
Baseline characteristics of a cohort of non-septic CSICU subjects diagnosed with systematic
inflammatory response syndrome by genotype of leucyl/cystinyl aminopeptidase (LNPEP)
rs10051637 (GG vs. AA/AG)

| | AA/AG | GG | Combined | Test |
|---|---|---|---|---|
| | (N = 56) | (N = 14) | (N = 70) | Statistic |
| AGE | 60.75/66.00/72.00 | 60.25/63.00/69.25 | 58.25/65.50/70.75 | F = 0.65 d.f. = 1.68 P = 0.423 |
| GENDER | 68% (38) | 64% (9) | 67% (47) | X\hat{ }2 = 0.06 d.f. = 1 P = 0.799 |
| SMOKER | 23% (13) | 29% (4) | 24% (17) | X\hat{ }2 = 0.17 d.f. = 1 P = 0.676 |
| DIABETES | 21% (12) | 14% (2) | 20% (14) | X\hat{ }2 = 0.36 d.f. = 1 P = 0.55 |
| H.TENSE | 54% (30) | 64% (9) | 56% (39) | X\hat{ }2 = 0.52 d.f. = 1 P = 0.47 |
| EJEC.FRAC | 0.50/0.50/0.60 | 0.35/0.50/0.60 | 0.50/0.50/0.60 | F = 0.41 d.f. = 1.66 P = 0.525 |
| BYPASS | 1.26275/1.65000/2.05800 | 1.51225/1.63350/ | 1.31700/1.65000/ | F = 0.4 d.f. = 1.68 P = 0.527 |
| | | 2.06225 | 2.05000 | |
| CLAMP | 0.86275/1.20850/1.67100 | 1.07900/1.33300/ | 0.92475/1.29150/ | F = 0.48 d.f. = 1.68 P = 0.489 |
| | | 1.61225 | 1.70000 | |
| APROTININ | 7% (4) | 7% (1) | 7% (5) | X\hat{ }2 = 0 d.f. = 1 P = 1 |

TABLE 6.6 summarizes important SNP-biomarker associations. Subjects with the LNPEP rs10051637 GG genotype showed a smaller change in serum GCSF levels from baseline to 3 hours post-surgery than subjects with the LNPEP rs10051637 AG or AA genotypes (P<0.001). Furthermore, LNPEP rs10051637 AA subjects were observed to have lower baseline interleukin-8 (IL8) levels (P=0.0443) 3 hours post-surgery. These findings suggest that non-septic SIRS subjects with the LNPEP rs10051637 GG genotype have a decreased chemokine (GCSF) and proinflammatory (IL-8) response after cardiopulmonary bypass.

TABLE 6.6
Biological plausibility of leucyl/cystinyl aminopeptidase association
using biomarkers in a cohort of non-septic CSICU subjects diagnosed
with systematic inflammatory response syndrome by genotype of
leucyl/cystinyl aminopeptidase (LNPEP) rs10051637 (GG vs. AA/AG).
Biomarkers are measured in pg/ml.

| | AA/AG | | Combined | |
|---|---|---|---|---|
| | (N = 56) | GG (N = 14) | (N = 70) | Test Statistic |
| GCSF.3 | 221/288/441 | 115/145/209 | 179/260/ | F = 15.7 d.f. = 1.68 |
| | | | 368 | P < 0.001 |
| GCSF.DIF | 207/279/424 | 103/138/181 | 161/249/ | F = 14.6 d.f. = 1.68 |
| | | | 365 | P < 0.001 |
| IL8.0 | 0.0/13.6/22.2 | 0.0/0.0/13.8 | 0.0/7.2/ | F = 4.2 d.f. = 1.68 |
| | | | 20.2 | P = 0.0443 |

4.1.4 LNPEP rs38041

TABLE 6.7 summarizes the baseline characteristics of 70 non-septic SIRS subjects who were successfully genotyped (GG/AG vs. AA) at LNPEP rs38041. No significant differences between the two genotype groups were detected on admission to the CSICU.

TABLE 6.7
Baseline characteristics of a cohort of non-septic CSICU subjects diagnosed with systematic
inflammatory response syndrome by genotype of leucyl/cystinyl aminopeptidase (LNPEP)
rs38041 (AA vs. GG/AG)

| | AA | AG/GG | Combined | Test |
|---|---|---|---|---|
| | (N = 18) | (N = 52) | (N = 70) | Statistic |
| AGE | 60.25/63.00/69.25 | 60.75/66.00/72.25 | 58.25/65.50/70.75 | F = 1.46 d.f. = 1.68 P = 0.231 |
| GENDER | 67% (12) | 67% (35) | 67% (47) | X\hat{ }2 = 0 d.f. = 1 P = 0.96 |
| SMOKER | 22% (4) | 25% (13) | 24% (17) | X\hat{ }2 = 0.06 d.f. = 1 P = 0.813 |
| DIABETES | 17% (3) | 21% (z,899 ) | 20% (14) | X\hat{ }2 = 0.17 d.f. = 1 P = 0.682 |
| H.TENSE | 61% (11) | 54% (28) | 56% (39) | X\hat{ }2 = 0.29 d.f. = 1 P = 0.593 |
| EJEC.FRAC | 0.50/0.55/0.60 | 0.48/0.50/0.60 | 0.50/0.50/0.60 | F = 0.02 d.f. = 1.66 P = 0.881 |
| BYPASS | 1.42075/1.63350/ | 1.30450/1.65000/2.17900 | 1.31700/1.65000/2.05000 | F = 0.12 d.f. = 1.68 P = 0.73 |
| | 2.0000 | | | |
| CLAMP | 0.93325/1.33300/ | 0.87475/1.20850/1.65425 | 0.92475/1.29150/1.70000 | F = 0.26 d.f. = 1.68 P = 0.608 |
| | 1.69600 | | | |
| APROTININ | 6% (1) | 8% (4) | 7% (5) | X\hat{ }2 = 0.09 d.f. = 1 P = 0.762 |

TABLE 6.8 summarizes important SNP-biomarker associations. Subjects with the AA genotype had a significantly smaller change in serum GCSF levels from baseline to three hours post-cardiopulmonary bypass (P=0.00226) and significantly lower baseline serum interleukin-8 (IL8) levels (P=0.0417) compared to subjects with LNPEP rs38041 AG or GG. These findings suggest that non-septic SIRS subjects with LNPEP rs38041 AA have a decreased chemokine (GCSF) response after cardiopulmonary bypass and lower baseline serum IL-8 levels.

TABLE 6.8
Biological plausibility of leucyl/cystinyl aminopeptidase association
using biomarkers in a cohort of non-septic CSICU subjects
diagnosed with systematic inflammatory response syndrome by
genotype of leucyl/cystinyl aminopeptidase rs38041 (AA vs. GG/AG).
Biomarkers are measured in pg/ml.

| | | GG/AG | Combined | |
|---|---|---|---|---|
| | AA (N = 18) | (N = 52) | (N = 70) | Test Statistic |
| GCSF.3 | 115/164/266 | 221/288/423 | 179/260/ | F = 10.7 d.f. = 1.68 |
| | | | 368 | P = 0.00168 |
| GCSF.DIF | 103/154/244 | 211/279/415 | 161/249/ | F = 10.1 d.f. = 1.68 |
| | | | 365 | P = 0.00226 |
| IL8.0 | 0.0/0.0/16.0 | 0.0/13.6/22.2 | 0.0/7.2/ | F = 4.31 d.f. = 1.68 |
| | | | 20.2 | P = 0.0417 |

4.2 Arginine Vasopressin (AVP)

4.2.1. AVP rs857242

TABLE 6.9 summarizes the baseline characteristics of 57 non-septic SIRS subjects who were genotyped at AVP rs857242. No significant differences between the genotype groups were detected on admission to the CSICU.

TABLE 6.9
Baseline characteristics of a cohort of non-septic CSICU subjects diagnosed with systematic
inflammatory response syndrome by genotype of Arginine Vasopressin (AVP) rs857242.

| | AC | CC | Combined | Test |
|---|---|---|---|---|
| | (N = 11) | (N = 57) | (N = 68) | Statistic |
| AGE | 60.50/65.00/71.00 | 60.00/65.00/72.00 | 58.25/65.50/70.75 | F = 0.04 d.f. = 1.66 P = 0.837 |
| GENDER | 64% (7) | 67% (38) | 66% (45) | Chisquare = 0.04 d.f. = 1 |
| | | | | P = 0.846 |
| SMOKER | 27% (3) | 23% (13) | 24% (16) | Chisquare = 0.1 d.f. = 1 |
| | | | | P = 0.749 |
| DIABETES | 9% (1) | 23% (13) | 21% (14) | Chisquare = 1.06 d.f. = 1 |
| | | | | P = 0.303 |
| H.TENSE | 64% (7) | 56% (32) | 57% (39) | Chisquare = 0.21 d.f. = 1 |
| | | | | P = 0.645 |
| EJEC.FRAC | 0.45/0.50/0.60 | 0.50/0.50/0.60 | 0.50/0.50/0.60 | F = 0.02 d.f. = 1.64 P = 0.897 |
| BYPASS | 1.0415/1.3330/1.9665 | 1.3670/1.6500/2.0830 | 1.3170/1.6500/2.0500 | F = 1.25 d.f. = 1.66 P = 0.268 |
| CLAMP | 0.78350/1.03300/1.65850 | 0.93300/1.25000/1.63300 | 0.92475/1.29150/1.70000 | F = 0.41 d.f. = 1.66 P = 0.525 |
| APROTININ | 9% (1) | 7% (4) | 7% (5) | Chisquare = 0.06 d.f. = 1 |
| | | | | P = 0.81 |

TABLE 6.10 summarizes important SNP-biomarker associations for AVP rs857242. Subjects with the AVP rs857242 CC genotype showed a strong trend towards a smaller change in GCSF levels at three hours post-cardiopulmonary bypass than subjects with the AVP rs857242 AC genotype (p=0.0978). These findings suggest that non-septic SIRS subjects with the AVP position rs857242 CC genotype have a decreased chemokine (GCSF) response after cardiopulmonary bypass surgery.

TABLE 6.10
Biological plausibility of Factor V association using biomarkers in a
cohort of non-septic CSICU subjects diagnosed with systematic
inflammatory response syndrome by genotype of Arginine
Vasopressin (AVP) rs857242. Biomarkers are measured in pg/ml.

| | | | Combined | |
|---|---|---|---|---|
| | AC (N = 11) | CC (N = 57) | (N = 68) | Test Statistic |
| GCSF.3 | 257\|319\|540 | 180\|255\|368 | 179\|260\|368 | F = 3.38 |
| | | | | d.f. = 1.66 |
| | | | | P = 0.0704 |
| GCSF.DIF | 257\|314\|519 | 169\|240\|368 | 161\|249\|365 | F = 2.82 |
| | | | | d.f. = 1.66 |
| | | | | P = 0.0978 |

### 4.3 Arginine Vasopressin Receptor 1a (AVPR1A)

4.3.1 AVPR1A rs1495027

TABLE 6.11 summarizes the baseline characteristics of 69 non-septic SIRS subjects who were successfully genotyped (CT/TT vs. CC) at AVPR1A rs1495027. Subjects with the CC genotype had shorter clamp time (P=0.03) than subjects with the CT/TT genotypes. There were no other significant differences prior to cardiopulmonary bypass surgery.

TABLE 6.11
Baseline characteristics of a cohort of non-septic CSICU subjects diagnosed with systematic
inflammatory response syndrome by genotype of arginine vasopressin receptor 1a (AVPR1A)
rs1495027 (CC vs. CT/TT).

| | CC | CT/TT | Combined | Test |
|---|---|---|---|---|
| | (N = 26) | (N = 43) | (N = 69) | Statistic |
| AGE | 58.50/64.50/68.50 | 61.00/66.00/73.00 | 58.25/65.50/70.75 | F = 1.41 d.f. = 1.67 |
| | | | | P = 0.239 |
| GENDER | 69% (18) | 67% (29) | 68% (47) | X\hat{ }2 = 0.02 d.f. = 1 |
| | | | | P = 0.877 |
| SMOKER | 19% (5) | 28% (12) | 25% (17) | X\hat{ }2 = 0.66 d.f. = 1 |
| | | | | P = 0.418 |
| DIABETES | 31% (8) | 14% (6) | 20% (14) | X\hat{ }2 = 2.83 d.f. = 1 |
| | | | | P = 0.0924 |
| H.TENSE | 46% (12) | 63% (27) | 57% (39) | X\hat{ }2 = 1.82 d.f. = 1 |
| | | | | P = 0.177 |
| EJEC.FRAC | 0.45/0.50/0.60 | 0.50/0.50/0.60 | 0.50/0.50/0.60 | F = 0.35 d.f. = 1.65 |
| | | | | P = 0.557 |
| BYPASS | 1.0955/1.4415/2.0330 | 1.4415/1.7330/2.0580 | 1.3170/1.6500/2.0500 | F = 3.29 d.f. = 1.67 |
| | | | | P = 0.0743 |
| CLAMP | 0.77100/0.97500/1.52075 | 1.06700/1.30000/1.73350 | 0.92475/1.29150/1.70000 | F = 4.64 d.f. = 1.67 |
| | | | | P = 0.0348 |
| APROTININ | 4% (1) | 9% (4) | 7% (5) | X\hat{ }2 = 0.72 d.f. = 1 |
| | | | | P = 0.397 |

TABLE 6.12 summarizes important SNP-biomarker associations for AVPR1A rs1495027. Subjects with the AVPR1A rs1495027 CC genotype were observed to have lower interleukin 8 (IL8) levels at baseline (p=0.046) and at three hours post cardiopulmonary bypass (p=0.0231) and had a strong trend towards smaller change in IL8 levels post-cardiopulmonary bypass surgery when compared to AVPR1A rs1495027 CT or TT subjects (P=0.0664). These findings suggest that non-septic SIRS Subjects with the AVPR1A rs1495027 CC genotype have a decreased pro-inflammatory cytokine (IL8) response at baseline and after cardiopulmonary bypass surgery. A trend towards lower MCP1 levels at baseline was also observed for subjects with the CC genotype compared with AVPR1A rs1495027 subjects with AVPR1A rs1495027 CT/TT genotypes P=0.09).

TABLE 6.12
Biological plausibility of arginine vasopressin receptor 1a association
using biomarkers in a cohort of non-septic CSICU subjects diagnosed
with systematic inflammatory response syndrome by genotype of
arginine vasopressin receptor 1a (AVPR1A) rs1495027
(CC vs. CT/TT). Biomarkers are measured in pg/ml.

| | | CT/TT | Combined | |
|---|---|---|---|---|
| | CC (N = 26) | (N = 43) | (N = 69) | Test Statistic |
| IL8.0 | 0.0/0.0/16.0 | 0.0/15.6/21.1 | 0.0/7.2/ | F = 4.13 d.f. = 1.67 |
| | | | 20.2 | P = 0.0461 |
| IL8.3 | 26.0/37.6/67.2 | 33.7/63.6/ | 27.9/44.9/ | F = 5.41 d.f. = 1.67 |
| | | 136.3 | 78.4 | P = 0.0231 |
| IL8.DIF | 21.6/27.2/58.9 | 24.4/47.7/ | 22.2/35.7/ | F = 3.48 d.f. = 1.67 |
| | | 116.1 | 67.0 | P = 0.0664 |
| MCP1.0 | 117/169/203 | 155/188/262 | 135/182/ | F = 2.83 d.f. = 1.67 |
| | | | 245 | P = 0.0973 |

4.3.2 AVPR1A rs3803107

TABLE 6.13 summarizes the baseline characteristics of the 70 non-septic SIRS subjects who were successfully genotyped (CT/TT vs. CC) at AVP position rs3803107. No significant differences were detected between the two genotype groups prior to cardiopulmonary bypass surgery.

TABLE 6.13
Baseline characteristics of a cohort of non-septic CSICU subjects diagnosed with systematic
inflammatory response syndrome by genotype of arginine vasopressin receptor 1a (AVPR1A)
rs3803107 (CT/TT vs. CC).

| | CC | CT/TT | Combined | Test |
|---|---|---|---|---|
| | (N = 49) | (N = 21) | (N = 70) | Statistic |
| AGE | 61.00/65.00/71.00 | 57.00/66.00/72.00 | 58.25/65.50/70.75 | F = 0.07 d.f. = 1.68 P = 0.79 |
| GENDER | 63% (31) | 76% (16) | 67% (47) | X\hat{ }2 = 1.11 d.f. = 1 |
| | | | | P = 0.291 |
| SMOKER | 22% (11) | 29% (6) | 24% (17) | X\hat{ }2 = 0.3 d.f. = 1 P = 0.584 |
| DIABETES | 20% (10) | 19% (4) | 20% (14) | X\hat{ }2 = 0.02 d.f. = 1 |
| | | | | P = 0.896 |
| H.TENSE | 51% (25) | 67% (14) | 56% (39) | X\hat{ }2 = 1.46 d.f. = 1 |
| | | | | P = 0.227 |
| EJEC.FRAC | 0.50/0.50/0.60 | 0.48/0.50/0.60 | 0.50/0.50/0.60 | F = 0.01 d.f. = 1.66 |
| | | | | P = 0.934 |
| BYPASS | 1.333/1.667/2.133 | 1.350/1.600/1.767 | 1.317/1.650/2.050 | F = 0.63 d.f. = 1.68 |
| | | | | P = 0.431 |
| CLAMP | 0.93300/1.30000/1.75 | 0.88300/1.13300/1.433 | 0.92475/1.29150/1.700 | F = 1.34 d.f. = 1.68 |
| | | | | P = 0.252 |
| APROTININ | 8% (4) | 5% (1) | 7% (5) | X\hat{ }2 = 0.26 d.f. = 1 |
| | | | | P = 0.613 |

TABLE 6.14 summarizes important SNP-biomarker associations for AVPR1A rs3803107. Subjects with the AVPR1A rs3803107 CC genotype had significantly higher serum MCP1 concentrations at baseline compared to those with AVPR1A rs3803107 CT or TT (P=0.0288). This finding suggests that the non-septic SIRS subjects with the AVPR1A rs3803107 CC genotype had higher MCP1 levels at baseline.

TABLE 6.14
Biological plausibility of arginine vasopressin receptor 1a association
using biomarkers in a cohort of non-septic CSICU subjects diagnosed
with systematic inflammatory response syndrome by genotype of
arginine vasopressin receptor 1a (AVPR1A) rs3803107
(CT/TT vs. CC). Biomarkers are measured in pg/ml.

| | | CT/TT | Combined | |
|---|---|---|---|---|
| | CC (N = 49) | (N = 21) | (N = 70) | Test Statistic |
| MCP1.0 | 162.2/187.2/ | 78.7/133.8/ | 134.9/182.0/ | F = 4.99 d.f. = 1.68 |
| | 261.5 | 223.4 | 245.2 | P = 0.0288 |

4.3.3 AVPR1A rs10877970

TABLE 6.15 summarizes the baseline characteristics of the 69 non-septic SIRS subjects who were successfully genotyped (CC/CT vs. TT) at AVPR1A rs10877970. No significant differences were detected between the two genotype groups prior to cardiopulmonary bypass surgery.

TABLE 6.15
Baseline characteristics of a cohort of non-septic CSICU subjects diagnosed with systematic
inflammatory response syndrome by genotype of arginine vasopressin receptor 1a (AVPR1A)
rs10877970 (CC/CT vs. TT).

| | CT/CC | TT | Combined | Test |
|---|---|---|---|---|
| | (N = 20) | (N = 49) | (N = 69) | Statistic |
| AGE | 57.00/66.50/70.50 | 61.00/65.00/72.00 | 58.25/65.50/70.75 | F = 0.29 d.f. = 1.67 |
| | | | | P = 0.591 |
| GENDER | 75% (15) | 63% (31) | 67% (46) | X\hat{ }2 = 0.88 d.f. = 1 |
| | | | | P = 0.348 |
| SMOKER | 25% (5) | 24% (12) | 25% (17) | X\hat{ }2 = 0 d.f. = 1 |
| | | | | P = 0.964 |
| DIABETES | 25% (5) | 18% (9) | 20% (14) | X\hat{ }2 = 0.39 d.f. = 1 |
| | | | | P = 0.534 |
| H.TENSE | 65% (13) | 51% (25) | 55% (38) | X\hat{ }2 = 1.12 d.f. = 1 |
| | | | | P = 0.290 |
| EJEC.FRAC | 0.405/0.550/0.600 | 0.500/0.500/0.600 | 0.500/0.500/0.600 | F = 0 d.f. = 1.65 P = 0.967 |
| BYPASS | 1.3250/1.6915/2.3210 | 1.3330/1.6500/2.0330 | 1.3170/1.6500/2.0500 | F = 0.01 d.f. = 1.67 |
| | | | | P = 0.917 |
| CLAMP | 0.87075/1.35850/1.600 | 0.93300/1.25000/1.717 | 0.92475/1.29150/1.700 | F = 0.14 d.f. = 1.67 |
| | | | | P = 0.714 |
| APROTININ | 5% (1) | 8% (4) | 7% (5) | X\hat{ }2 = 0.21 d.f. = 1 |
| | | | | P = 0.646 |

TABLE 6.16 summarizes important SNP-biomarker associations for AVPR1A rs10877970. Subjects with the AVPR1A rs10877970 TT genotype showed a trend towards higher serum MCP levels (P=0.0865) at baseline compared to subjects with AVPR1A rs10877970 CT or CC. This finding suggests that non-septic SIRS subjects who carry either the AVPR1A rs10877970 CT or CC genotypes had lower MCP1 levels at baseline.

TABLE 6.16
Biological plausibility of arginine vasopressin receptor 1a association
using biomarkers in a cohort of non-septic CSICU subjects diagnosed
with systematic inflammatory response syndrome by genotype of
arginine vasopressin receptor 1a (AVPR1A) rs10877970
(CC/CT vs. TT). Biomarkers are measured in pg/ml.

| | CT/CC | | Combined | |
|---|---|---|---|---|
| | (N = 20) | TT (N = 49) | (N = 69) | Test Statistic |
| MCP1.0 | 76.4/148.8/ | 162.2/187.2/ | 134.9/182.0/ | F = 3.05 d.f. = 1.67 |
| | 236.0 | 249.6 | 245.2 | P = 0.0856 |

### SUMMARY

Numerous discoveries described herein show that single nucleotide polymorphisms of the vasopressin (AVP rs1410713, rs857240, rs857242) gene, the arginine vasopressin A1 receptor (AVPR1A rs1495027) gene, and the leucyl/cystinyl aminopeptidatase (LNPEP rs18059, rs2771 I, and rs10051637) gene are associated with response (measured as survival, organ dysfunction and need of life support) to AVP.

Furthermore, markers in the vasopressinase gene (LNPEP rs18059, rs27711, and rs10051637) and the vasopressin A1 receptor gene (AVPR1A rs1495027) are also markers of increased use of AVP in a cohort of critically ill subjects who have septic shock. Accordingly, clinicians more frequently administer infused AVP to subjects who have LNPEP genotypes rs18059 CC, rs27711 AA and rs10051637 GG and subjects who have the AVPR1A genotype, rs1495027 CT. These genotypes also have a significantly decreased chance of survival when treated with infused AVP compared to comparable subjects who have septic shock but who are not infused with AVP (control).

In a separate study of an independent cohort of subjects with cardiopulmonary bypass surgery, we have also found that LNPEP rs18059 CC, LNPEP rs27711 AA and LNPEP rs10051637 GG are associated with decreased inflammatory response (measured as GCSF and IL-8 response) to non-septic causes of systemic inflammatory response syndrome (subjects having cardiopulmonary bypass surgery).

The clinical utility of these discoveries is that before subjects who have SIRS, sepsis or septic shock and other inflammatory conditions listed below are considered for treatment with a vasopressin receptor agonist, they may be genotyped for single nucleotide polymorphisms of the vasopressin (AVP) gene (rs1410713, rs857240, and rs857242), the vasopressin A1 receptor (AVPR1A) gene (rs1495027), and the vasopressinase (LNPEP) gene (rs18059, rs27711 and rs10051637). Subjects who have AVP rs857240 CT or rs857242 AC genotypes; the AVPR1A rs1495027 TT genotype, or the LNPEP rs18059 CC, rs27711 AA or rs10051637 GG genotypes should not receive vasopressin receptor agonist(s) (e.g. V-1 receptor agonist, e.g. a Via receptor agonist, e.g. an AVPR1 agonist) because vasopressin receptor agonist(s) dramatically decreases their survival and increases the risk of organ dysfunction.

Similarly, before subjects who have SIRS, sepsis or septic shock and the conditions listed below are considered for treatment with any vasopressin receptor agonist(s), they should be genotyped for single nucleotide polymorphisms of the vasopressin (AVP) gene (rs1410713, rs857240 and rs857242), the vasopressin A1 receptor (AVPR1A) gene (rs1495027), and the vasopressinase (LNPEP) gene (rs18059, rs27711 and rs10051637). Subjects who have the AVP rs1410713 AA or AC, rs857240 CC or rs857242 CC genotypes; the AVPR1A rs1495027 CC genotype, and the LNPEP rs18059 TT or rs27711 GG genotypes should receive vasopressin receptor agonist(s) (e.g. V-1 receptor agonist, e.g. a Via receptor agonist, e.g. an AVPR1 agonist) because vasopressin receptor agonist(s) dramatically increases their survival and decreases the risk of organ dysfunction.

Furthermore, subjects undergoing or having cardiac surgery (of all types in all ages and hypotensions), cardiac surgery requiring cardiopulmonary bypass, cardiac surgery not requiring cardiopulmonary bypass, cardiac transplantation and hypotension, dialysis-induced hypotension, autonomic neuropathy, trauma and hypotension are also likely to be administered a vasopressin receptor agonist and should also be genotypes for single nucleotide polymorphisms of the vasopressin (AVP) gene (rs1410713, rs857240, and rs857242), the vasopressin A1 receptor (AVPR1A) gene (rs1495027), and the vasopressinase (LNPEP) gene (rs18059, rs27711 and rs10051637).

Similarly, before subjects who have pregnancy-associated diuresis, diabetes insipidus and are considered for treatment with vasopressin, they should be genotyped for single nucleotide polymorphisms of the vasopressin (AVP) gene (rs1410713, rs857240, and rs857242), the vasopressin A1 receptor (AVPR1A) gene (rs1495027), and the vasopressinase (LNPEP) gene (rs18059, rs27711 and rs10051637).

TABLE 7.1 shows that subjects who have the LNPEP rs18059 CC, rs27711 AA or rs10051637 GG genotypes (P=0.0398 interaction statistic of LNPEP rs18059 TT and AVP infusion and survival) who receive AVP infusion have decreased survival compared to subjects who have the LNPEP rs18059 CC, rs27711 AA or rs10051637 GG genotypes who do not receive AVP infusion.

Furthermore. TABLE 7.1 shows that subjects who carry the LNPEP rs18059 CC genotype have a significantly increased chance of receiving AVP infusion than subjects who do not carry the LNPEP rs18059 CC genotype (p=0.0257). Furthermore, subjects who carry the LNPEP rs27711 AA genotype have a significantly increased chance of receiving AVP infusion than subjects who do not carry the LNPEP rs27711 AA genotype (p=0.0033). Furthermore, subjects who carry the LNPEP rs10051637 GG genotype have a significantly increased chance of receiving AVP infusion than subjects who do not carry the LNPEP rs10051637 GG genotype (p<0.001).

TABLE 7.1
Summary of Key Results of SNPs, Alleles and Genotypes of the Vasopressinase Gene (LNPEP)

| | INCREASE | | | | | | |
|---|---|---|---|---|---|---|---|
| | IN USE OF | | SURVIVAL | | | BIOLOGICAL | |
| LNPEP SNP | VASO | GROUP | (%) BY GENOTYPE | | | PLAUSIBILITY | P |
| rs18059 | Geno = CC | | CC | CT | TT | CC: Smaller | 0.003 |
| | | | | | | increase of GCSF | |
| | P = 0.0257 | CONT | 67 | 28 | 15 | | |
| | | VASO | 44 | 36 | 38 | | |
| | | Sig (P < 0.05) | | | 0.0398 | | |
| | | Interaction | | | | | |
| rs27711 | Geno = AA | | AA | AG | GG | AA: Smaller | <0.001 |
| | | | | | | increase of GCSF | |
| | P = 0.0033 | CONT | 60 | 36 | 19 | AA: Smaller | 0.05 |
| | | | | | | increase of IL-8 | |
| | | VASO | 43 | 36 | 33 | | |
| rs10051637 | Geno = GG | | GG | AG | AA | GG: Smaller | 0.001 |
| | | | | | | increase of GCSF | |
| | P < 0.001 | CONT | 60 | 35 | 20 | GG: Smaller | 0.04 |
| | | | | | | increase of IL-8 | |
| | | VASO | 46 | 38 | 26 | | |

In addition, subjects who have the LNPEP rs18059 CC genotype have a less pronounced rise in GCSF after cardiac surgery (p=0.003). In addition, subjects who carry the LNPEP rs27711 AA genotype have a less pronounced rise in GCSF (p=0.001) and IL-8 (p=0.05) after cardiac surgery. In addition, subjects who have the LNPEP rs10051637 GG genotype have a less pronounced rise in GCSF (p=0.001) and IL-8 (p=0.04) after cardiac surgery.

TABLE 7.2 shows that subjects who have the AVP rs1410713 CC, AVP rs857240 CT, and AVP rs857242 AC genotypes who receive AVP infusion have decreased survival compared to subjects who have the AVP rs1410713 CC, AVP rs857240 CT, and AVP rs857242 AC genotypes who do not receive AVP infusion.

TABLE 7.2
Summary of Key Results of SNPs, Alleles and Genotypes of the
Vasopressin Gene (AVP).

| | | SURVIVAL | | | | |
|---|---|---|---|---|---|---|
| | | (%) BY | | | BIOLOGICAL | |
| AVP SNP | GROUP | GENOTYPE | | | PLAUSIBILITY | P |
| rs1410713 | | CC | AC | AA | | |
| | CONT | 35 | 37 | 0 | | |
| | VASO | 32 | 47 | 38 | | |
| rs857240 | | CT | CC | | | |
| | CONT | 43 | 30 | | | |
| | VASO | 29 | 41 | | | |
| rs857242 | | AC | CC | | | |
| | CONT | 54 | 30 | | AC: INCREASED | 0.07 |
| | | | | | GCSF | |
| | VASO | 38 | 41 | | | |

Subjects who have the AVP rs857242 AC genotype have a greater rise in GCSF (p=0.07) after cardiac surgery than subjects who do have the AVP rs857242 CC genotype.

TABLE 7.3 shows that subjects who have the AVPR1A rs1495027 TT genotype (P=0.0466 interaction statistic of AVPR1A rs1495027 TT and AVP infusion and survival) who receive AVP infusion have decreased survival compared to subjects who have the AVPR1A rs1495027 TT genotype who do not receive AVP infusion.

TABLE 7.3
Summary of Key Results of SNPs, Alleles and Genotypes of the AVPR1 Gene.

| | INCREASE | | SURVIVAL | | | | |
|---|---|---|---|---|---|---|---|
| | IN USE OF | | (%) BY | | | BIOLOGICAL | |
| AVPR1 SNP | VASO | GROUP | GENOTYPE | | | PLAUSIBILITY | P |
| rs1495027 | Geno = CT | | TT | CT | CC | | |
| | P = 0.0240 | CONT | 46 | 35 | 24 | CT/TT: Greater | 0.06 |
| | | | | | | increase IL-8 | |
| | | VASO | 23 | 38 | 50 | | |
| | | Sig (P < 0.05) | | | 0.0466 | | |
| | | Interaction | | | | | |

Furthermore, TABLE 7.3 shows that subjects who carry the AVPR1A rs1495027 CT genotype have a significantly increased chance of receiving AVP infusion than subjects who do not carry the AVPR1A rs1495027 CT genotype (p=0.0240).

Subjects who have the AVPR1A rs1495027 CT/TT genotypes have a greater rise in IL-8 (p=0.06) after cardiac surgery than subjects who do have the AVPR1A rs1495027 CC genotype.

Although the foregoing invention has been described in some detail by way of illustration and example for purposes of clarity of understanding, it will be readily apparent to those of skill in the art in light of the teachings of this invention that changes and modification may be made thereto without departing from the spirit or scope of the appended claims.

1. A method for obtaining a prognosis for a subject having, or at risk of developing, an inflammatory condition, the method comprising determining a genotype of said subject which includes one or more polymorphic sites in the subject's vasopressin pathway gene sequences or a combination thereof, wherein said genotype is indicative of an ability of the subject to recover from the inflammatory condition, wherein the polymorphic site is
one or more of rs18059; rs27711; rs38041; rs10051637; rs1410713; rs857240; rs857242; rs10877970; rs3803107; and rs1495027; or
one of the following polymorphic sites in linkage disequilibrium thereto: rs2762; rs10051637; rs1477364; rs7731592; rs7736466; rs1363974; rs2351010; rs1423357; rs1544777; rs2161548; rs38032; rs38034; rs38041; rs27436; rs27306; rs27307; rs27397; rs27659; rs27711; rs27290; rs38030; rs27294; rs27747; rs39602; rs248215; rs27302; rs2278018; rs1559355; rs3734015; rs4869315; rs2247650; rs2549781; rs2549782; rs2161657; rs251339; rs187265; rs2548527; rs1056893; rs2548523; rs2255546; rs2255637; rs1019503; rs251344; rs1981846; rs10071975; rs7700332; rs38042; rs18059; rs9127; rs7972829; rs10784339; rs3803107; rs11836346; rs7308008; rs11835545; rs7959001; rs11832877; rs10877977; rs2201895; rs7302323; rs10877986; rs2030106; rs1495027; rs10877962; rs1042615; rs16856; rs18059; rs27296; rs27300; rs27613; rs27711; rs38033; rs38035; rs38036; rs38041; rs38043; rs716848; rs1216565; rs1230358; rs1363907; rs1974871; rs2042385; rs2113050; rs2113189; rs2161658; rs2255633; rs2255634; rs2287988; rs2548524; rs2548529; rs2548530; rs2548532; rs2548533; rs2548536; rs2548538; rs2548539; rs2548540; rs2549783; rs2549784; rs2549790; rs2549791; rs2549794; rs2549795; rs2549796; rs2549797; rs2617447; rs2910686; rs2927609 rs3797796; rs3849749; rs3849750; rs4360063; rs4869314; rs4869316; rs6556942; rs7713127; rs7716222; rs7719705; rs10044354; rs10051637; rs10058476; rs12516666; or rs12716486.

2.-4. (canceled)

5. The method of claim 1, further comprising obtaining vasopressin pathway gene sequence information for the subject.

6. The method of claim 1, wherein the genotype is determined using a nucleic acid sample from the subject.

7. The method of claim 6, further comprising obtaining the nucleic acid sample from the subject.

8. The method of claim 1, wherein said genotype is determined using one or more of the following techniques:
(a) restriction fragment length analysis;
(b) sequencing;
(c) micro-sequencing assay;
(d) hybridization;
(e) invader assay;
(f) gene chip hybridization assays;
(g) oligonucleotide ligation assay;
(h) ligation rolling circle amplification;
(i) 5′ nuclease assay;
(j) polymerase proofreading methods;
(k) allele specific PCR;
(l) matrix assisted laser desorption ionization time of flight (MALDI-TOF) mass spectroscopy;
(m) ligase chain reaction assay;
(n) enzyme-amplified electronic transduction;
(o) single base pair extension assay; and
(p) reading sequence data.

9. The method of claim 1, wherein the genotype of the subject is indicative of increased risk of death or organ dysfunction from the inflammatory condition
wherein the genotype comprises at least one of the following risk genotypes: rs18059CT; rs18059TT: rs27711GA; rs27711GG: rs38041GA: rs38041GG: rs10051637GA; rs10051637GG; rs1410713AA; rs857240CC; rs857242CC; rs10877970CC; rs3803107TT; and rs1495027TT; or
wherein the genotype comprises at least one of risk alleles rs3803107T or rs10877970C; or a polymorphic site in linkage disequilibrium thereto selected from one or more of the polymorphic sites and corresponding genotypes set out in TABLES 1B and 1D.

10.-12. (canceled)

13. The method of claim 1, wherein the genotype of the subject is indicative of decreased risk of death or organ dysfunction from the inflammatory condition
wherein the genotype comprises at least one of the following reduced risk genotypes: rs18059CC; rs27711AA; rs38041AA; rs10051637AA; rs1410713CC; rs1410713AC; rs857240TT; rs857240CT; rs857242AA: rs857242AC; rs10877970TT; rs10877970CT; rs3803107CC; rs3803107CT; rs1495027CC and rs1495027CT; or
wherein the genotype comprises at least one of reduced risk alleles rs3803107C or rs10877970T; or a polymorphic site in linkage disequilibrium thereto selected from one or more of the polymorphic sites and corresponding genotypes set out in TABLES 1B and 1D.

14.-18. (canceled)

19. The method of claim 1, wherein the inflammatory condition is selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects, subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglottitis, E. coli 0157:H7, malaria, gas gangrene, toxic shock syndrome, pre-eclampsia, eclampsia, HELLP syndrome, mycobacterial tuberculosis, Pneumocystis carinii pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

20. The method of claim 19, wherein the inflammatory condition is selected from one or more of the following: SIRS, sepsis and septic shock.

21. A method for selecting a group of subjects for determining the efficacy of a candidate drug known or suspected of being useful for the treatment of an inflammatory condition, the method comprising:
(i) determining a genotype at one or more polymorphic sites in a vasopressin pathway gene sequence for each subject, wherein said genotype is indicative of the subject's ability to recover from the inflammatory condition, and
(ii) sorting subjects based on their genotype.

22. The method of claim 21 further comprising, administering the candidate drug to the subjects or a subset of subjects and determining each subject's ability to recover from the inflammatory condition.

23. The method of claim 22, further comprising comparing subjects' responses to the candidate drug according to the subjects' genotype.

24. A method of treating an inflammatory condition in a subject in need thereof, comprising:
(a) administering a vasopressin receptor agonist to said subject if he has an improved response genotype in his their vasopressin pathway-associated gene sequence, or
(b) selectively refraining from administering a vasopressin receptor agonist to said subject if he has an adverse response genotype in his vasopressin pathway-associated gene sequence.

25.-30. (canceled)

31. The method of claim 24, further comprising determining the number of organ system failures for the subject as an assessment of subject risk.

32. The method of claim 31, wherein two or more organ system failures are indicative of increased subject risk.

33. The method of claim 24, wherein the inflammatory condition is selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglottitis, E. coli 0157:H7, malaria, gas gangrene, toxic shock syndrome, pre-eclampsia, eclampsia, HELLP syndrome, mycobacterial tuberculosis, Pneumocystis carinii pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

34. The method of claim 24, wherein the inflammatory condition is SIRS, sepsis or septic shock.

35. The method of claim 24, wherein the improved response genotype is found at one or more of the following polymorphic sites: rs18059; rs27711; rs10051637; rs1410713; rs857240; rs857242; and rs1495027; or
a polymorphic site in linkage disequilibrium thereto selected from the group consisting of: rs2762; rs10051637; rs1477364; rs7731592; rs7736466; rs1363974; rs2351010; rs1423357; rs1544777; rs2161548; rs38032; rs38034; rs38041; rs27436; rs27306; rs27307; rs27397; rs27659; rs27711; rs27290; rs38030; rs27294; rs27747; rs39602; rs248215; rs27302; rs2278018; rs1559355; rs3734015; rs4869315; rs2247650; rs2549781; rs2549782; rs2161657; rs251339; rs187265; rs2548527; rs1056893; rs2548523; rs2255546; rs2255637; rs1019503; rs251344; rs1981846; rs10071975; rs7700332; rs38042; rs18059; rs9127; rs7972829; rs10784339; rs3803107; rs11836346; rs7308008; rs11835545; rs7959001; rs11832877; rs10877977; rs2201895; rs7302323; rs10877986; rs2030106 and rs18059; rs27296; rs27300; rs27613; rs27711; rs38033; rs38035; rs38036; rs38041; rs38043; rs716848; rs1216565; rs1230358; rs1363907; rs1974871; rs2042385; rs2113050; rs2113189; rs2161658; rs2255633; rs2255634; rs2287988; rs2548524; rs2548529; rs2548530; rs2548532; rs2548533; rs2548536; rs2548538; rs2548539; rs2548540; rs2549783; rs2549784; rs2549790; rs2549791; rs2549794; rs2549795; rs2549796; rs2549797; rs2617447; rs2910686; rs2927609 rs3797796; rs3849749; rs3849750; rs4360063; rs4869314; rs4869316; rs6556942; rs7713127; rs7716222; rs7719705; rs10044354; rs10051637; rs10058476; rs12316666; and rs12716486.

36. (canceled)

37. The method of claim 35, wherein the improved response genotype is one or more of the following: rs18059CT; rs18059TT; rs27711GG; rs10051637GA; rs10051637AA; rs1410713AC; rs1410713AA; rs857240CC; rs857242CC; rs1495027CC; and rs1495027CT; or
is a polymorphic site in linkage disequilibrium thereto that is one or more of the polymorphic sites and corresponding genotypes set out in TABLES 1B and 1D.

38. (canceled)

39. The method of claim 37, wherein:
(a) the vasopressin receptor agonist is selectively administered when the subject has an improved response genotype, or
(b) the vasopressin receptor agonist is selectively not administered when the subject has an adverse response genotype selected from the group consisting of:
(i) rs18059CC: rs27711AA; rs10051637GG; rs1410713CC; rs857240CT; rs857242AC; and rs1495027TT or
(ii) a polymorphic site in linkage disequilibrium thereto set out in TABLES 1B and 1D.

40.-42. (canceled)

43. The method of claim 24, wherein the vasopressin receptor agonist is vasopressin.

44. Two or more oligonucleotides or peptide nucleic acids of about 10 to about 400 nucleotides that hybridize specifically to a sequence contained in a human target sequence consisting of a subject's vasopressin pathway-associated gene sequence, a complementary sequence of the target sequence or RNA equivalent of the target sequence and wherein the oligonucleotides or peptide nucleic acids are operable in determining the presence or absence of two or more polymorphisms in the subject's vasopressin pathway associated gene sequence which polymorphisms are at
(i) one of polymorphic sites rs18059; rs27711; rs38041; rs10051637; rs1410713; rs857240; rs857242; rs10877970; rs3803107; or rs1495027; or
(ii) one or more of the following polymorphic sites in linkage disequilibrium thereto: rs2762; rs10051637; rs1477364; rs7731592; rs7736466; rs1363974; rs2351010; rs1423357; rs1544777; rs2161548; rs38032; rs38034; rs38041; rs27436; rs27306; rs27307; rs27397; rs27659; rs27711; rs27290; rs38030; rs27294; rs27747; rs39602; rs248215; rs27302; rs2278018; rs1559355; rs3734015; rs4869315; rs2247650; rs2549781; rs2549782; rs2161657; rs251339; rs187265; rs2548527; rs1056893; rs2548523; rs2255546; rs2255637; rs1119503; rs251344; rs1981846; rs10071975; rs7700332; rs38042; rs18059; rs9127; rs7972829; rs10784339; rs3803107; rs11836346; rs7308008; rs11835545; rs7959001; rs11832877; rs10877977; rs2201895; rs7302323; rs10877986; rs2030106; rs1495027; rs10877962; rs1042615; rs16856; rs18059; rs27296; rs27300; rs27613; rs27711; rs38033; rs38035; rs38036; rs38041; rs38043; rs716848; rs1216565; rs1230358; rs1363907; rs1974871; rs2042385; rs2113050; rs2113189; rs2161658; rs2255633; rs2255634; rs2287988; rs2548524; rs2548529; rs2548530; rs2548532; rs2548533; rs2548536; rs2548538; rs2548539; rs2548540; rs2549783; rs2549784; rs2549790; rs2549791; rs2549794; rs2549795; rs2549796; rs2549797; rs2617447; rs2910686; rs2927609 rs3797796; rs3849749; rs3849750; rs4360063; rs4869314; rs4869316; rs6556942; rs7713127; rs7716222; rs7719705; rs10044354; rs10051637; rs10058476; rs12516666; or rs12716486.

45. (canceled)

46. Two or more oligonucleotides or peptide nucleic acids selected from the group consisting of:
(a) an oligonucleotide or peptide nucleic acid that hybridizes under high stringency conditions to a nucleic acid molecule comprising SEQ ID NO:1 having a T at position 201 but not to a nucleic acid molecule comprising SEQ ID NO:1 having a C at position 201;
(b) an oligonucleotide or peptide nucleic acid that hybridizes under high stringency conditions to a nucleic acid molecule comprising SEQ ID NO:1 having a C at position 201 but not to a nucleic acid molecule comprising SEQ ID NO:1 having a T at position 201;
(c) an oligonucleotide or peptide nucleic acid that hybridizes under high stringency conditions to a nucleic acid molecule comprising SEQ ID NO:2 having a G at position 201 but not to a nucleic acid molecule comprising SEQ ID NO:2 having a A at position 201;
(d) an oligonucleotide or peptide nucleic acid that hybridizes under high stringency conditions to a nucleic acid molecule comprising SEQ ID NO:2 having an A at position 201 but not to a nucleic acid molecule comprising SEQ ID NO:2 having a G at position 201;
(e) an oligonucleotide or peptide nucleic acid that hybridizes under high stringency conditions to a nucleic acid molecule comprising SEQ ID NO:3 having an A at position 201 but not to a nucleic acid molecule comprising SEQ ID NO:3 having a G at position 201;
(f) an oligonucleotide or peptide nucleic acid that hybridizes under high stringency conditions to a nucleic acid molecule comprising SEQ ID NO:3 having a G at position 201 but not to a nucleic acid molecule comprising SEQ ID NO:3 having an A at position 201;
(g) an oligonucleotide or peptide nucleic acid that hybridizes under high stringency conditions to a nucleic acid molecule comprising SEQ ID NO:4 having a G at position 201 but not to a nucleic acid molecule comprising SEQ ID NO:4 having an A at position 201;
(h) an oligonucleotide or peptide nucleic acid that hybridizes under high stringency conditions to a nucleic acid molecule comprising SEQ ID NO:4 having an A at position 201 but not to a nucleic acid molecule comprising SEQ ID NO:4 having a G at position 201;
(i) an oligonucleotide or peptide nucleic acid that hybridizes under high stringency conditions to a nucleic acid molecule comprising SEQ ID NO:5 having an A at position 201 but not to a nucleic acid molecule comprising SEQ ID NO:5 having a C at position 201;
(j) an oligonucleotide or peptide nucleic acid that hybridizes under high stringency conditions to a nucleic acid molecule comprising SEQ ID NO:5 having a C at position 201 but not to a nucleic acid molecule comprising SEQ ID NO:5 having an A at position 201;
(k) an oligonucleotide or peptide nucleic acid that hybridizes under high stringency conditions to a nucleic acid molecule comprising SEQ ID NO:6 having an T at position 201 but not to a nucleic acid molecule comprising SEQ ID NO:6 having a C at position 201;
(l) an oligonucleotide or peptide nucleic acid that hybridizes under high stringency conditions to a nucleic acid molecule comprising SEQ ID NO:6 having a C at position 201 but not to a nucleic acid molecule comprising SEQ ID NO:6 having an T at position 201;
(m) an oligonucleotide or peptide nucleic acid that hybridizes under high stringency conditions to a nucleic acid molecule comprising SEQ ID NO:7 having an A at position 201 but not to a nucleic acid molecule comprising SEQ ID NO:7 having a C at position 201;
(n) an oligonucleotide or peptide nucleic acid that hybridizes under high stringency conditions to a nucleic acid molecule comprising SEQ ID NO:7 having a C at position 201 but not to a nucleic acid molecule comprising SEQ ID NO:7 having an A at position 201;
(o) an oligonucleotide or peptide nucleic acid that hybridizes under high stringency conditions to a nucleic acid molecule comprising SEQ ID NO:8 having a T at position 201 but not to a nucleic acid molecule comprising SEQ ID NO:8 having a C at position 201;
(p) an oligonucleotide or peptide nucleic acid that hybridizes under high stringency conditions to a nucleic acid molecule comprising SEQ ID NO:8 having a C at position 201 but not to a nucleic acid molecule comprising SEQ ID NO:8 having a T at position 201;
(q) an oligonucleotide or peptide nucleic acid that hybridizes under high stringency conditions to a nucleic acid molecule comprising SEQ ID NO:9 having a C at position 201 but not to a nucleic acid molecule comprising SEQ ID NO:9 having a T at position 201;
(r) an oligonucleotide or peptide nucleic acid that hybridizes under high stringency conditions to a nucleic acid molecule comprising SEQ ID NO:9 having a T at position 201 but not to a nucleic acid molecule comprising SEQ ID NO:9 having a C at position 201;
(s) an oligonucleotide or peptide nucleic acid that hybridizes under high stringency conditions to a nucleic acid molecule comprising SEQ ID NO:10 having a T at position 201 but not to a nucleic acid molecule comprising SEQ ID NO:10 having a C at position 201;
(t) an oligonucleotide or peptide nucleic acid that hybridizes under high stringency conditions to a nucleic acid molecule comprising SEQ ID NO:10 having a C at position 201 but not to a nucleic acid molecule comprising SEQ ID NO:10 having a T at position 201;
(u) an oligonucleotide or peptide nucleic acid that hybridizes under high stringency conditions to a nucleic acid molecule comprising a first allele for a given polymorphism selected from the polymorphisms listed in TABLE 1D but not capable of hybridizing under high stringency conditions to a nucleic acid molecule comprising a second allele for the given polymorphism selected from the polymorphisms listed in TABLE 1D; and
(v) an oligonucleotide or peptide nucleic acid that hybridizes under high stringency conditions to a nucleic acid molecule comprising the second allele for a given polymorphism selected from the polymorphisms listed in TABLE 1D but not capable of hybridizing under high stringency conditions to a nucleic acid molecule comprising the first allele for the given polymorphism selected from the polymorphisms listed in TABLE 1D.

47. An array of oligonucleotides or peptide nucleic acids attached to a solid support, the array comprising two or more of the oligonucleotides or peptide nucleic acids of claim 44.

48. A composition comprising an addressable collection of two or more oligonucleotides or peptide nucleic acids, which consists essentially of two or more nucleic acid molecules of SEQ ID NO:1-264 or complements, fragments, variants, or analogs thereof.

49. The oligonucleotides or peptide nucleic acids of claim 44, further comprising one or more of the following: a detectable label; a quencher; a mobility modifier; a contiguous non-target sequence situated 5′ or 3′ to the target sequence or 5′ and 3′ to the target sequence.
